SEQUENCE LISTING (216 sequences)

SEQ ID NO: 1 - DNA, 5382 bp, Unknown
  Description of Unknown adaA sequence
atgtctgcccctacgaagctggtctttttcggaaacgaattccccaacgatgatctcaaagccctcttccgcggtctgcaccgacacggcaaggacaggcgcttccgccagctggcaaccttcttggaggagtccactcgcgtgcttcagaatgaagtcgcccaacttcccgagccgctaaaaaagctggtgcctcactttgagaacctgatgcccctaaccgaggttgatttccgccagggacctctgggagctgctatggaaagcgccctcctcacaattctcgaactgggaatgttcatcggccactacgaagccgaggagcgtgtctgggacctctccgcagatcgggccaccttggctggtttgagtattggcctgctcgctgctgctggtgtcgctctgtctactcacttggctgaggtggtccagaacggcgctgagtgtgtgcgggtttctttccgcttgggagtctacgtccacgacatctcccgcaagctcgaagctccccaggcagacggcagcttgctcagctgggctcatgttgtcaccggtgagacagcatccgacctgcaggaggaactgtcgcgatacaatacggagactggcactcccgagctgctcaaggtctttatcagtgccgctgataagacctctgtgagtgtcagtggccctccctcgcgcatccgtgcggccttccgggcctctcagcgcctgcgctactccaagtctctcgccctgcccgtgtatgacggcctctgccatgccgcccatctatacgacgaggagactatccaccgtgttcttcatccagatggatccgtcatccctacttcccggccggtgcaactggcactcctctcatcgcgctctggccagcccttcgaggccaccacggccgctgagctgttccgcgccatcagcacagaactgctgactggcaccatcttcttggacaatatcacggccggtattctcgaccgcactgaacgctgtgccgacgccacacagtgccagatcgaaacctatcgcacttcgctggtgttcaagggtctgctgaaagctctggaggcttgcttccccgatcggaccatcagcactaccgatctcatcccctgggtgttccaggactatggtgcgcgccagcccaagtcatgcgcagactcgaagctggccatcgtcggcatggcctgccgcatgcccggaggtgccaatgatctcgacctcttctgggagcttctcgcacagggtcgtgatacgcatacgacggtgcctgcggatcgcttcgacctcgaaacacactatgatccgaccggtgagacagagaacgccacacgcacgccctttggcaacttcattgaccagccgggacttttcgatgcgggattcttcaacatgtctcctcgcgaggccgagcagaccgatcccatgcaccgcctcgcccttgtcacagcatacgaagccctagaaatggcgggtatcgtttctggccgcacgccatcatccaaccctaagcgcatcgccaccttctacggacaggccagtgacgactggcgcgagcttaatgcctcccagaatatcggcacctacgccgtccctggtggtgaacgtgcctttgccaacggtcgcattaactacttcttcaagtttggtggcccatctttcaatctcgacacggcttgctccagcggtctggctgccgtccaggcggcctgctctgctctgtgggctggtgaagccgacactgtcctggccggtggactgaacattatcaccgaccccgacaactacgccggtctgggcaatggccacttcctgtcgcgcaccggccagtgcaaggtttgggaccagtctgctgacggttactgccgtgcagatggtgtgggatctgtggtcatcaagcgccttgaggacgcggaggctgacaatgacaacatcctggctgtcgtgctctctgctgccaccaaccactcggcggaggccatctctatcactcacccccatgctggcgcccagaaggagaactacacccaggtgctgcatcaagctgccgtcaatccactggacataagctatgtcgagctgcacggcactggcacccaggccggagatgcccaggaggctgagtcggtcttggacatctttgccccacgaaaccatcgtcgtcgcgccgatcagcctctgcacttgggcgcggtgaaaagtaacattggtcatggtgaggcagctgctggtattgcctccctgctcaaggtgctgctcatgtaccagaagaacgagattcctgcgcacatcggcattcccacagtcatcaacccggctatccccaccgatcttgagcagcgcaaggtttatttgccacgcacaaagactgcctggccccgggctgcaggccagatccgtcgcgctatcgtgaactcttttggcgcgcacggtggtaatactaccctggtgctggaagatgcccctgagaagcaggtgacggtggcccgtgaggagcgctcgacgcaccctgtcgtcatctccgccaagtcgaagaagtccctggccgccaacgtggagactctccttgcctacctcgacgagaacccggagactgacctcggcgacctgtcctacacgacctgtgcccgccgcatgcaccacagctggcgtctggccactgccgtcagcgacattcccgccctgcagaagttcctgcgcaacgctgtgagcaacgatgccgtttcccagacccggcccattcccaccgaggctccccctgtcgtgttcactttcaccggccagggcgcctactacgccgggctggcacagggtttgttccaggctctgcctttcttccgcgccgaggtgcgccagctggaccacctgtcccagcgcctgggattcccctcgatcgtgccggtcatccttggcgaggtggaagagggcactgctacggccctggtcacgcagcttagcattgtgatcgtggagattgccctggccaggctctggctactcctcctaggcattccggctcctcacgccgtgatcggccacagtctcggcgaatacgccgcattggctgtcgctggagtgctgtccacggcagacgctctctacctggtcggccaccgggctcagctcattgaggaacactgcacccccggcagtcacgccatgctctcggtgcgcgcaaccatcgctgacatcgagcgtctggtgggcaccggctctgatgcaccgacttacgagctgtcctgccaaaacacgcaccaggacaccgtcatcggcggctcaatccaggacctcaatgccatccgggagaagctcgagcatgaaggaatcaagtgtgtcaacgtcgatgtcccctttgccttccacaccgcgcagatggacgccgtccgggaacgcctcgccaaggccgtcgccgccgtgcccttcaagacccccagcgtccctgttctctcgcctctgctgggcagcgtcgtctttgacggcaagtctatcaacccagagtacatcgtgcgggcaactcgcgaacccgtccagtttgcgactgccatcgacgccgcccaggaactcggcatcgtcaacagccagaccctctgggtcgacatcggcccccaccccatctgcgccagtttcgtgcgcagtctggtgcctggcgcacgcatcgtttcgtcctgccgccgtaacgaggacaactttgcaaccatggccaagagtctctgcacactgcacctggccggccgcaccccctcctgggccgagtacttccgtcccgacgagcaggcttactctctcctccggctgcccaagtaccgctggaacgaggtcaactactggatccagtacctgggcacctggaccctggacaaggcgcacctcaagaacggcggcagtcagaagcgcgccatcaccgatgtaccatctatctcatccctccgcacctccctcatccaccaggtcaccgaggagactgtcgacaagaccaccgccaccctcaaggccatctccgacatccagcaccccgacttcctcgaagctgtgcacggccacaccatgaacaactgcggcgtcgccacctcctccatctggaccgacatggccatgaccgtcggcgagcacctctaccgtcgcttggtccctggcactgaccacgtcctcatggacctctgcgactttgaagtccagcacgctcaagtcgccaacaccaactcaaacaccccgcaacccctcgccctcgaagcccacctcgacctccccacccgccacatgtccctcgcctggtacgacgtcaatgccacgaccaaccagcgcgccgatgcccccttcgccaccggcagcatcaagtaccctgccgacccaaccggcgcagcctggtccatcgaatggtcccgcatcacgcacctcattcagggccgcatcgaagccctgcagcacctggccgcggagaacaaagccagcactctgtccaaacccctcgcctacgctctcttcaagaacgtcgtcgactacgccccccgctaccgcggcatggaccgcgttgtcattcacgaccacgaggccttctccgatatcacccttaccaccgaccgccacggaacctggcacaccccgccccactggatcgacagcgtttcgcacctggccggcctggtcatgaacggcagcgatgcttccaacaccagggacttcttctacgtcacccctggctgcagcagctgccgcatggctgagcctttgattgctggaggcaagtaccggaactatgtccgcatgttccccatgccggacgaggcgcacatgtatgcgggcgacttgtacatcctccgcgaggacaagatcattggtgtcgtcgaacagctcaagttccgcagagtgccccgcttgttgatggatcgcttcttctcgccgaataagaatgccgcggcgcatgctgctcctgctcctgctcctgctgcggttccagccgtgaagaagcaacctcccactgaaaccatccagccccaggcccccaagacagagcagaagcaagaccagctacaacttcccaatctcgcctccgctgcaccctccaccgccagcagctcatcctcaccctcatccagcggcgtcgctacccccactactgagcaagaagcccctggcgctgatgcctcagcggtgaccggcgtggccggcaaatgtctggaactgattgccaacgagacaggcctgggtgtggcggaattgactgcggacgcgacattcgtgcagctgggtgtggattcgctgatgtcgctggtgctgtcggagaagttgagaagtgagatgggattggagattaagagttcgttgttcttggagtgtcctactgtgggagatttgacggggtggttggagcagtattgctaa

SEQ ID NO: 2 - DNA, 954 bp, Unknown
  Description of Unknown adaB sequence
atggccttccgtatccccttcgctcaatctttctggcaagaatacctgtccggccaggaagccaacctgcctcgtctacccgaagtcgagcaagtcaccgagacagtaatgcgcattctcggaggtaatcccgggcgcatgcagctgcaagggaccaacacgtacctcgtggggacgggcaaattccgaattctcattgacaccggccagggtgaagcaagctggatcgaagccctcacgaaacaacttgaagccaatggtctagaaatttcccatgtcttgctcactcactggcacggagatcacaccggaggggtccccgacctcatcacctacaaccccgaactatcctcacgagtatacaaaaacacccccgatctcggccaacaagccatccacgatggtcagaaattccacgtagagggcgcaactatccgtgctgtctttacccccggccacgcctttgatcacatgtgcttcctgctggaggaagaaaatgccctcttcactggggacaacgtgctaggacatggatactctgtcgtggaagatctgggcacatatatgaccagtctaacacgcatggcggacctcaactgcgcgcttgggtatccagctcatgggacgcgtatcgaggatcttcctgccaagatgaaggaatatatccagcataaggagtcaaggatgcggcaggtgctggccgccttggagaggagtcgggcgaggatgacggcaactgggggtgggcgccgtgcgggtgcgctgacattcccggagcttataaactctatgtatggagggattccagatgagattgaacaggctttgacgccgttcttgagtcaggttttgtggaaattggcggaggatcggaaggttgggtttgagggcgagccgagtcagagaagatggtttgcggttgggccgccagctgcaactgcagtgaggctgtaa

SEQ ID NO: 3 - DNA, 1245 bp, Unknown
  Description of Unknown adaC sequence
atgacccctcccatcctcatcatcggcgccggcctctccggcctcaccatctcccgcatcctcaccaacgcctccatccccaacatcgtctttgaagcctccacccccgaccgcagccaaggctacgccatcagcctccgcgaatggggctacacctccctcctcacagccctaggtgacctgcccctccgcagtctcacccggggcgtggctccagaccgcatcctcggcggaaccggctggatcgaccaagccctccgggacaaccacaccggaaacctcctagtggctcccgacccagaagccaagcaatgcatcgtgcgggcaaatcgcaacgccctccgcacctggatcgccgatagcggtgacgaggaagtggacatccgctacggccaccgtctgcgcagtgtgcagggctcaatgggaaacgtgacggccacgttcgacaacggcgccaagtaccagggctcattggtcatcgcagcagatggcgtgcattctagcgtgcgttctcagatcctgccccacgtgagcccggatattgtgcccgtggtggtgtatcatggggaattggaactccctcgcaaggaattcgataatcttatccgtccgcactccggaccgtcgaatatcctagctggtgtgggcgatgggttcaatacccctattactgtttgtaatatcactcctacacatgtgcatctcgattggtcgtattctagaccgagtacggaaaacaaggagaataaggatcctctctatcggccgcatgtctctgctgctgaggctaagcagattcccccggccttgttggaggaaattgcctcccgggatctggcgaggccgtggtcccagctgcttaatgccgaggcgttgccgactcatcgtgtctttaactgggtgagtcgctgtgtgtcggtaacgagggaggatgtgaatgctgcgcagaagcagggcgtggtgtttattggggactcgtggcatgcgatgccaatttttgggggcgaagggggtaatcatgccctggttgatgcggtggagttggcggaggcattgacgggtaaggaagggaatttggatgctgctgtaacggggtattatgatcgcgcgtggagaagatgtcaggaggccgtgagaagatctaggcagaggttcttccagttgcatcggcctatgagggagtggatggagattgcggagaagaaaaagatgatggctgctatgaagggggtggaggctcattaa

SEQ ID NO: 4 - DNA, 750 bp, Unknown
  Description of Unknown adaD sequence
atgtcctcagtcaccttaaccacaaccacaaccaccacctccaccccacccaaacccacccccaaagacgaaccccaagaacaaatctacaccccctggcgcctcttcatctacgacatctgggtcctcggcatcgtgagcaccctcgcctggggctgccggatcagcacctacctaatccccttattccgctccaacgtgggcaaaaagcacctcgacatcggcgccggcaccggctactacctcaaccaagcccggatctcctcaacaacccagctcacaatcgtcgacaacgaaacccacgcgctcaacgtcgccctcgctcgctgcaagcacccagtcacccaaacgcacggcatcgtcaccgacatcctgcaaccctcgcccttcccagaaacctacctcaccaacaatgaccaaaaattcgactccgtatcaatgtactacctcctgcactgcctgcccgtgcccgtggccagcaaatgcaagatcttcacgcatctcaagaaatacatgacagaggatggggtggttcatggagcgaatgtgctgggcaagggggtgaggaaggataattggtttgcgaggattatccgaagagggtgtttgaatcatggggtgttccataatgaggaggataatgcgtatgagtttgagagggccctcagggagaacttttgggaggtggagacttgggtggtcgggagtgtttttgtttttagggccaagaggccgattcttgatgcttaa

SEQ ID NO: 5 - DNA, 1035 bp, Unknown
  Description of Unknown NpgA sequence
atggtgcaagacacatcaagcgcaagcacttcgccaattttaacaagatggtacatcgacacccgccctctaaccgcctcaacagcagcccttcctctccttgaaaccctccagcccgctgatcaaatctccgtccaaaaatactaccatctgaaggataaacacatgtctctcgcctctaatctgctcaaatacctcttcgtccaccgaaactgtcgcatcccctggtcttcaatcgtgatctctcgaaccccagatccgcacagacgaccatgctatattccaccctcaggctcacaggaagacagcttcaaagacggatataccggcatcaacgttgagttcaacgtcagccaccaagcctcaatggtcgcgatcgcgggaacagcttttactcccaatagtggtggggacagcaaactcaaacccgaagtcggaattgatattacgtgcgtaaacgagcggcagggacggaacggggaagagcggagcctggaatcgctacgtcaatatattgatatattctcggaagtgttttccactgcagagatggccaatataaggaggttagatggagtctcatcatcctcactgtctgctgatcgtcttgtggactacgggtacagactcttctacacttactgggcgctcaaagaggcgtatataaaaatgactggggaggccctcttagcaccgtggttacgggaactggaattcagtaatgtcgtcgccccggccgctgttgcggagagtggggattcggctggggatttcggggagccgtatacgggtgtcaggacgactttatataaaaatctcgttgaggatgtgaggattgaagttgctgctctgggcggtgattacctatttgcaacggctgcgaggggtggtgggattggagctagttctagaccaggaggtggtccagacggaagtggcatccgaagccaggatccctggaggcctttcaagaagttagatatagagcgagatatccagccctgtgcgactggggtgtgtaattgcctatcctaa

SEQ ID NO: 6 - DNA, 1875 bp, Aspergillus niger
atggcccgccggaacagcatacgcgggatcacagacgagcagcacggccggtatagatccagctcgccgctcgcccgcactgccatcgcccgcgatattgaggactacgccgacgacgaaggatccatgctcaccaccgacgacgaagcctccgaaacctccaccatccgcgccattaacagccagcccggcacgaatccccattcgctggccgggtcgtaccagcggcccggattcttcactaccgtatcgcacgcgacggttgtgccgcatcggccggataacgaggggttaacgcggcgggagcgggagcgggccattgaggatgagcggaatcttctcactgataatcgatgcattgagccgggtgtgcagaagggagggcgcatgaggcgcgggtcgggtgtggaagcgacggaaacggctgcattgttgggcgggcagcgtcgcggatcacagtatgaaacggttgaggatcaggaggagattgatcggaagtgggaggaggctgttaccgctgggttgattcagacgacgtggaagcgtgaagctcaggtcattgggaagaatgccgcgccgttggtggttacctttctgctgcagtattcgttgaccgtggcgagtatttttaccctcgggcatttgggcaagaaggagcttggggccgttagtttggctagtatgagtgcgagtattacgggttatgctgtttaccaaggtctggcgacgagtttggataccctgtgtgcgcaggcgtatggctccgggaggaagaagctggtaggtctgcagatgcaaaagatggtattcttcctctgggctatctccatacctattattttgttgtggttcttcgccgaccgcatcctcgtccggatcgtgccggaaagagaggtcgccatgctggcgggcttgtacttgaaggtggttgcgctgggtgctccagggtacgcttgcttcgagagtggcaagcggttcgtgcaggcgcagggactcttctctgcctcgctttatgtgcttctcatctgtgccccgttgaatgcagtgatgaactacgtattcgtatggcagttcggctggggcttcatcggagctcccatcgctgtagccatcacagacaacctgatgcctctcttcctcttcctgtacgtgtacttcatcgacggctccgaatgctggaacggcgtcaccacccgcgccctacgcaactggggccccatgatcaagctcgcccttcccgggctcctcatggtcgaagccgaatgtctcgcctttgaagttctgaccctggcgtcctcgtatctcggcaccaccccattagccgcccaatccatcctgtccaccatctccagcatcaccttccaaatccccttccccgtatccatctccggtagcacccgcgtcgccaacctcatcggcgcgacgcttgtcgacgccgccaagctatccgccaaggtatccatgattggcgccgtcatcgtcgggctgctgaacatgctcctcctctcttccctgcgctactacatcccgtacctgttcacctcggacgaggaggtcattgagctcgtcgcccaggtcctgcctctctgtgcggcgttccagctctttgatgccttggctgccaactgtaacggaattctgcgcggtatcggtcgccaggaaattggcggctatgtgcagctgttttgctactatgctattgctatgccaattagtttcggcacgacatttggattgaactggggcttgtttggactgtggtctggtgttgcgttggccttgttattggtatcggtcattgaggcgttctttttgacgcagacgaattggcatcgctcggtggaggatgcgctgcgccggaatgcgatgacttaa

SEQ ID NO: 7 - DNA, 1515 bp, Aspergillus niger
atgtacgactctcttcctagctaccgggagacttcttccgcgcacactcatggagaggaacatacgccactacttcccaagcaggtcgacaccggccccgactccaaaagcgcaaaatcatcggtttcctttctggtcgaatttttccggctgttgaaggactccataccagtcattctggcatacacgctgcaaaacagcctacaaaccacctcggtgttgattgtgggacggacctctccggagaacctggccaccacagctttctcactgatgtttgccatggtcacggcatggatgatcgccttaggcgggaccaccgcattggacacactagcctcctcaacgttcacaggcagctcgaataagcatgatctcggaattctgttgcagcgggcattctttgtcctcggcctgttctatgtccccgttgcaattctttggacatgctcggagcctgtgtttcttctgcttggccaggacccccagctttcacgggatagtgcgcgtttcctaacttgcttgatccctggcggtctgggttatatctacttcgaggccatgaaaaaatatctgcaagcacagggtattatgcgccctgggacgtatgtcttgctgattacggtgccgtttaatgcattgctcaattacctgttctgttacacctttcgcatggggttgcttggtgcgccatttgccaccggcatttcctactggctgtcctttgcactgctcgtgttgtatgcccgattcattgccggctccgaatgctggggcggctggtcgcgcaaagcattcgagaatcttgggactttcgcccgattggcctttctgggcgtggtgcatgtaggcaccgagtggtgggcctttgagatagtagctcttgcagccggaaggctgggaacaatcccactggcggctcagagcgtcatcatgaccgcagaccaagtgctcaacacaatcccatttggcgtgggagtcgctacatcttcacgggtggggagcctgctaggctcccgggatgcagctggtgcatcgagagcggccaacaccgcggcctggctaagtatggcgctgggaggggccgtgctggctgtgctgatggggactcgacacgtctttgcaaagatattcaactccgatgaaggggtcgtacagctcactgcggaggttctaccatgggtggcgttgttccagatcgccgatggattgaacggtagctgtggaggaagccttaggggtatgggaaggcagcatgtcggcgcgctagtcaacctggccagttattattgtggtgcactacctctaggtatttggctagccttcaatggatggggactgaaaggtctttgggtgggacagtgcatcgcattgtatcttgtgggggcactggagtggacaatcgtggccttcagtaactgggaaggcgaggtcgataaggcgtttcagcgaatggatatccatgaccgattggaggttggacacaccaccaatggtgccacgactgttgtgtaa

SEQ ID NO: 8 - DNA, 1668 bp, Aspergillus niger
atgacggacgagaagaatgcccgggagctaacggccacccctgtcgacttggatgaactgccggccggatactactgcaggccagaccccagctacagcttgatctcgaccgttttcacactgatctccggagtcggtctactcttggtaggccgcttaggagatctctttggccgccgatacatcctgattggagggcaactacttggccttatcggcgccatagtctgtgcaacagccaaaaccgttcccaccgttatcggaggcagtgtcctctgcggtctggctgctgcagtgcaattgactttcagcttcgtgatcgctgagctggtccccaaccgtgctcgtccggtcgtcaatgctggcatcttcatcacaacgctgcccttctcagcgttcggttctctgattgcagatctgttcatcgccaacaccgcccgctcctggcgctggacttactacctgaacatcatcacctgcggtctttctatcattttgctggtgctgttttacttccctccaggctgggatgccaagcgcggaaatgagagccgcatcgatggcctcaagaaattcgactacattggctttctcctatacgccggcggactgatcctggtcctgcttggcctctgtgagtctcccgttccagctcttactcggtttttccatggtgctaattcaatcccatcagcatggggcggctcatcgtatgcatggcactcagcccatgtcatcgctgttctggtcattggattcgtctgcttgctcgcttttgccctatatgaaatattcgtccccttgcagcagcctctcctgcccatgtccttactccgaaaccgcggatacgccggtgcggtgtgctcggccctagtaggaaacatggtctacttctccatgagtctcctctggccaaccgccatcgccgacctctacaccacaaacaccatcaaaacgggctggctttcaatctccaccggcgccggcgtcatcgtcggcgaagtcgccgccggcatcctgatgaaacccgtgggatacacaaaataccaactgatcatcaccaccctcatgatcaccgccttctccggggccctagcatccattaaccaataccgccaggcatacggcatcgcgttcaccgccctcggcggcttcttcgtcggctacctcgaactgataaccataatcatgtgccccctgtattgctcaccccaggacatcgggctagccagcggcttcctcggctccgcaaagcaagtcgccggcaccatcgccaccgccatctacgtcgccatcctggacaaccgcgtcacggtggacttgccgcgcgatgtatctgctgccgcattgaacgcaggactgccttcatcctcgctgacggatctgctggaggcggtgtcggcggggacgacggcagcgtatgaggctgttcccggcatgacgaatggtattctgggggctgtgacagaggcggtgaaaacggcgtattcgcagtcgtttcgcacggtgtttttggcgagtattgcgtttggcggactgtcggtgatcgcggcgtgttttgccgagggggtggatgggaggtttacgggcgatgtggcggctaggttgaagggtgtggatggtggtggttctggaagtgaggagaagttgaaggatgggaaggtttaa

SEQ ID NO: 9 - DNA, 1377 bp, Aspergillus niger
atggacgaccgcgaaaaccccaaaaactggagcatcagcaagaaatgggctgtgaccatcaccgtgtcgctcttcacattcatttcccccgtttcatcctccatgatctcacccgccttgaccaagatcgccaaagaactcaacatcacatcgcaagtcgaatccgaactaagtctctctgttttcgtgctcgcctacgccgtcggcccactctgcttcggcccgctatccgaactctttggtcgtgcctacgtcctgcaatgcagtaatctatgctacgtggcgtggaaccttggatgcggcttcgcgcagacatcggggcagttgatcgcgtttcgtttcatgagtgggatagcaggtagtgcgccgttagctattggaggtggtgttctcagtgactgctggaacgccgaccaacgcggcaaagccgtcggcatctacagcctggccccccttctcggtcccgtcgtgggccccatcgcgggtggcttcatcgccgaaacgacgacctggcggtgggttttctgggcgacaagcatcgccggcgccgtgatccaactaatcgggctggccttcctgcccgagacatatgcaccgacattgctcaaacgacgcgcgcggaccctgcgccagaaaactggcgacgagggataccacacggaagcagacacgcagaacgcaagcctgatgtccacattgcagcgcgctctcgtccgcccattcatcctcctcaccacgcagcccattgtgcaagtcatcgcgctgtacatggcgtatctcttcggattgttttacctgttgctctcgaccttccctcaagtctgggaggaggtctacggcgagagcgtcggcattggcgggctgaactacatttccctaggcattggatttgtgttgggagcgcaggtgaacgcgcgcgccagcgaccgcatctacaaacagctcaaggccaagcggaataacaccggactacccgagttccgtatcccggctatgtttgtcggctcattgtttatcccggtcgggttgttctggtatggatggagtgtgcaggcacatatccactggattatgcccaatatcggtattgtgttgtttagtattgggagcattatttgtctgcagtgtatgcagacgtacgtgatcgatagttatacgcggtttgcggcgagtgggttggctgctgcggtcgtgttgaggagtctggcgggctttagtttcccgttgtttgcgccgtatatgtatgataagctgcactatggctgggggaatagtttgcttggatttttgagtatcgggattggagtgccggcgccttgggtgttttggttctggggggcgaagttgagggggatgtcgaggtatgcttctggctaa

SEQ ID NO: 10 - DNA, 1716 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactagacacccatatgcattgtttgttccacaagtacgaactgaagaactgctaacaggtagagctttggagctaggggcggagctgcgtcgtggtcatgccgtgaccgccttggaacaagatgctgatggtgttactgtgagcgtgacaggccctgaaggcccatacgaagtagaatgtgcttatttggtgggctgcgacggtggaggcagtacggtgaggaaactattgggcatagattttccaggtcaagacccacatatgtttgctgtcatcgcagacgcaagattcagagaagagcttcctcacggagaaggtatgggtcctatgcgtccttatggtgtcatgagacatgaccttcgtgcatggttcgcagcatttccgctagaaccagacgtctacagagcaacagtcgcctttttcgatagaccgtatgctgacaggagggcgccggtaacggaggaggatgtcagagccgcgttaacagaagttgctggatctgactttggaatgcatgatgtaagatggttatctcgtttgaccgatacaagtagacaagcagaaaggtatagagatgggagagttcttttggctggtgatgcatgccatattcatttgcccgctggtggccagggactgaacttaggatttcaagatgccgttaacttgggttggaagcttggtgctaccattgccgggaccgctccaccagagttattggatacgtatgaagctgagagaaggccgatagccgccggtgttttgagaaatacaagggctcaagctgttctaattgatccagatcctcgctacgagggtttaagggaattaatgattgaattgttgcacgttcctgagactaatagatatttagcggggctaatctccgcattagacgttagatacccaatggctggggaacatccattgctgggaagaagagtacccgacttacctcttgtcaccgaagatggaacaagacagttgtccacttatttccatgctgcacgtggcgtattattaacgttaggttgtgatcaaccactagcagatgaagccgctgcttggaaagatagggttgatttagttgcagctgaaggtgtggcggaccctggttctgcagtagatggcttaactgctttacttgtaaggcctgatggttacatttgttggacagctgccccagaaactggtactgatggattgacagacgcgctgcgtacttggttcggcccacctgcaatggtatcgatggattacaaggatgacgacgataagatctgagctcttaattaacaattcttcgccagaggtttggtcaagtctccaatcaaggttgtcggct

SEQ ID NO: 11 - DNA, 1974 bp, Artificial Sequence
  Description of Artificial Sequence Synthetic polynucleotide
ccctcactaaagggaacaaaagctggagctcagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatggctgagttgaaacttggctacaaggcatcagctgaacagtttgccccaagggagttagtcgaactagcagtcgctgccgaagctcacggtatggattccgctactgtttccgaccatttccaaccatggagacaccaaggtggccatgcaccattctcactcagttggatgacagctgttggagaaagaactaatagattgttattggggacgtcggtactcaccccgacgtttagatacaaccctgcggtaatagcacaggcctttgctacaatgggatgtctatatccaaaccgggtgtttttaggtgttggtactggagaggccttgaatgaaatcgccactggttatgaaggtgcttggcctgagttcaaagaaaggtttgcgcgtctgcgcgaaagcgtggggctgatgagacaattatggtctggtgatagggtagattttgatggagattattatagattaaaaggcgcgtctatatatgacgttccggatggtggtgtccctgtatatattgccgccggaggaccagcagttgctaaatatgctggccgagctggtgacggtttcatatgcacttcaggcaagggtgaagaactttacacagaaaagttgatgcccgccgtcagagaaggggcggcagccgctgacaggtctgttgatggcatagacaaaatgattgagatcaagatttcatacgacccagatcccgaattagcaatgaataacacaagattttgggcacctcttagtttgaccgcagaacaaaagcatagcatcgatgatccaattgaaatggaaaaagctgctgatgctttacccatcgagcaaattgcaaaaagatggattgttgcaagtgatcctgatgaagcagtggagaaagtaggacagtacgtgacctggggtttaaatcatctagttttccacgctcctgggcatgatcaaagaagattcttggaattgtttcaatctgacctagcgccaagacttcgtcgtctgggttaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 12 - DNA, 1602 bp, Artificial Sequence
  Description of Artificial Sequence Synthetic polynucleotide
ccctcactaaagggaacaaaagctggagctcagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgagggtagctttacttggtggtacaggaaatcttggaaaaggccttgccctacgactggccacgctgggccatgaaatcgtagtcggaagcagaagagaagaaaaggcagaggctaaagcagcagaatataggcgcattgcaggtgacgcttccatcactggtatgaaaaatgaagatgccgctgaggcatgcgacattgctgtcttgaccattccttgggaacatgctattgacactgccagagatttgaagaatattttacgtgagaaaattgttgtatcaccattagttccagtgtcaagaggtgcaaagggcttcacctactcgtccgaaagatcagcggccgagattgtggctgaagttctggaatctgaaaaagttgtttctgcgttgcacacaatacctgctgcaagatttgccaacttggatgaaaaatttgattgggatgttcctgtttgtggtgatgatgacgaaagtaagaaagtcgtcatgtctttaataagtgaaatagatgggttgaggccgttagatgctggtcccctctctaactcccgtttagtggagagcctaactccattgatattgaacatcatgagattcaatggaatgggtgaactagggatcaagtttttataactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 13 - DNA, 1674 bp, Artificial Sequence
  Description of Artificial Sequence Synthetic polynucleotide
ccctcactaaagggaacaaaagctggagctcagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgactactcaagacagtgggtctgcaccgaagcctcccgccaaagatccatgggatttgccagatgtcagcgcactgtctgttggtgtcctaggtggtacaggaccacagggcaggggattagcctacagattggcgcgtgctggtcaaaaagtgaccttgggctcaagagatgctggacgcgctgctgatgcagcggcagagctgggccatggtgtggaaggtactgataatgcagaatgcgcaagaagatccgacatcgttattgttgctgtaccttgggacggtcatgctaaaactttggaaagtttaagagaagagttggctggcaagctggttatcgactgtgttaaccccttggggtttgataagaaaggtgcttatgctttaaaaccagaggagggctcggccgctgaacaagctgcggcgcttctccctgattcacgagtaacagcagctttccaccatttatctgctgtgcttttacaagatgaatccattgaagaaattgataccgatgttttggtcttaggtgaagcaagggcagacacggatattgtccaggcactagcagggcgtataccaggtatgagaggaatatttgccggtcgtctaagaggtgcccaccaagttgaatcacttgtagccaatttaatatctgtaaacagaaggtataaggctcatgccggactaagggccacagacgtgtaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 14 - DNA, 1149 bp, Artificial Sequence
  Description of Artificial Sequence Synthetic polynucleotide
tggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgccattcactcaaaaggagatcacgtatttaagggctcaaggctacggccgactagccaccgtcggtgctcacggtgaacctcacaacgtgccggtatcttttgaaattgatgaagaaagaggtaccattgaaataacaggaagggatatgggacgttccagaaaattcagaaatgttgccaaaagtgacagagtggcatttgttgttgatgatgttccatgcagagacccagaagttgtcagagctgttgtaatacatggtactgcacaggcgcttcccacaggcggaagagaaaggcgtcctcattgtgctgacgaaatgattagaattcatccaagaagaatagtaacatggggtatcgaaggtgatttgtcaactggtgttcatgcaagagatattactgctgaagatggtggtagaagggtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcgg

SEQ ID NO: 15 - DNA, 1509 bp, Artificial Sequence
  Description of Artificial Sequence Synthetic polynucleotide
atgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactagacacccatatgcattgtttgttccacaagtacgaactgaagaactgctaacaggtagagctttggagctaggggcggagctgcgtcgtggtcatgccgtgaccgccttggaacaagatgctgatggtgttactgtgagcgtgacaggccctgaaggcccatacgaagtagaatgtgcttatttggtgggctgcgacggtggaggcagtacggtgaggaaactattgggcatagattttccaggtcaagacccacatatgtttgctgtcatcgcagacgcaagattcagagaagagcttcctcacggagaaggtatgggtcctatgcgtccttatggtgtcatgagacatgaccttcgtgcatggttcgcagcatttccgctagaaccagacgtctacagagcaacagtcgcctttttcgatagaccgtatgctgacaggagggcgccggtaacggaggaggatgtcagagccgcgttaacagaagttgctggatctgactttggaatgcatgatgtaagatggttatctcgtttgaccgatacaagtagacaagcagaaaggtatagagatgggagagttcttttggctggtgatgcatgccatattcatttgcccgctggtggccagggactgaacttaggatttcaagatgccgttaacttgggttggaagcttggtgctaccattgccgggaccgctccaccagagttattggatacgtatgaagctgagagaaggccgatagccgccggtgttttgagaaatacaagggctcaagctgttctaattgatccagatcctcgctacgagggtttaagggaattaatgattgaattgttgcacgttcctgagactaatagatatttagcggggctaatctccgcattagacgttagatacccaatggctggggaacatccattgctgggaagaagagtacccgacttacctcttgtcaccgaagatggaacaagacagttgtccacttatttccatgctgcacgtggcgtattattaacgttaggttgtgatcaaccactagcagatgaagccgctgcttggaaagatagggttgatttagttgcagctgaaggtgtggcggaccctggttctgcagtagatggcttaactgctttacttgtaaggcctgatggttacatttgttggacagctgccccagaaactggtactgatggattgacagacgcgctgcgtacttggttcggcccacctgcaatg

SEQ ID NO: 16 - DNA, 1681 bp, Artificial Sequence
  Description of Artificial Sequence Synthetic polynucleotide
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatggctgagttgaaacttggctacaaggcatcagctgaacagtttgccccaagggagttagtcgaactagcagtcgctgccgaagctcacggtatggattccgctactgtttccgaccatttccaaccatggagacaccaaggtggccatgcaccattctcactcagttggatgacagctgttggagaaagaactaatagattgttattggggacgtcggtactcaccccgacgtttagatacaaccctgcggtaatagcacaggcctttgctacaatgggatgtctatatccaaaccgggtgtttttaggtgttggtactggagaggccttgaatgaaatcgccactggttatgaaggtgcttggcctgagttcaaagaaaggtttgcgcgtctgcgcgaaagcgtggggctgatgagacaattatggtctggtgatagggtagattttgatggagattattatagattaaaaggcgcgtctatatatgacgttccggatggtggtgtccctgtatatattgccgccggaggaccagcagttgctaaatatgctggccgagctggtgacggtttcatatgcacttcaggcaagggtgaagaactttacacagaaaagttgatgcccgccgtcagagaaggggcggcagccgctgacaggtctgttgatggcatagacaaaatgattgagatcaagatttcatacgacccagatcccgaattagcaatgaataacacaagattttgggcacctcttagtttgaccgcagaacaaaagcatagcatcgatgatccaattgaaatggaaaaagctgctgatgctttacccatcgagcaaattgcaaaaagatggattgttgcaagtgatcctgatgaagcagtggagaaagtaggacagtacgtgacctggggtttaaatcatctagttttccacgctcctgggcatgatcaaagaagattcttggaattgtttcaatctgacctagcgccaagacttcgtcgtctgggttaactcgag

SEQ ID NO: 17 - DNA, 1309 bp, Artificial Sequence
  Description of Artificial Sequence Synthetic polynucleotide
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgagggtagctttacttggtggtacaggaaatcttggaaaaggccttgccctacgactggccacgctgggccatgaaatcgtagtcggaagcagaagagaagaaaaggcagaggctaaagcagcagaatataggcgcattgcaggtgacgcttccatcactggtatgaaaaatgaagatgccgctgaggcatgcgacattgctgtcttgaccattccttgggaacatgctattgacactgccagagatttgaagaatattttacgtgagaaaattgttgtatcaccattagttccagtgtcaagaggtgcaaagggcttcacctactcgtccgaaagatcagcggccgagattgtggctgaagttctggaatctgaaaaagttgtttctgcgttgcacacaatacctgctgcaagatttgccaacttggatgaaaaatttgattgggatgttcctgtttgtggtgatgatgacgaaagtaagaaagtcgtcatgtctttaataagtgaaatagatgggttgaggccgttagatgctggtcccctctctaactcccgtttagtggagagcctaactccattgatattgaacatcatgagattcaatggaatgggtgaactagggatcaagtttttataactcgag

SEQ ID NO: 18 - DNA, 1381 bp, Artificial Sequence
  Description of Artificial Sequence Synthetic polynucleotide
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgactactcaagacagtgggtctgcaccgaagcctcccgccaaagatccatgggatttgccagatgtcagcgcactgtctgttggtgtcctaggtggtacaggaccacagggcaggggattagcctacagattggcgcgtgctggtcaaaaagtgaccttgggctcaagagatgctggacgcgctgctgatgcagcggcagagctgggccatggtgtggaaggtactgataatgcagaatgcgcaagaagatccgacatcgttattgttgctgtaccttgggacggtcatgctaaaactttggaaagtttaagagaagagttggctggcaagctggttatcgactgtgttaaccccttggggtttgataagaaaggtgcttatgctttaaaaccagaggagggctcggccgctgaacaagctgcggcgcttctccctgattcacgagtaacagcagctttccaccatttatctgctgtgcttttacaagatgaatccattgaagaaattgataccgatgttttggtcttaggtgaagcaagggcagacacggatattgtccaggcactagcagggcgtataccaggtatgagaggaatatttgccggtcgtctaagaggtgcccaccaagttgaatcacttgtagccaatttaatatctgtaaacagaaggtataaggctcatgccggactaagggccacagacgtgtaactcgag

SEQ ID NO: 19 - DNA, 1689 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataaaatctgagctcttaattaacaattcttcgccagaggtttggtcaagtctccaatcaaggttgtcggct

SEQ ID NO: 20 - DNA, 1125 bp, Artificial Sequence
  Description of Artificial Sequence Synthetic polynucleotide
tggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacggtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcgg

SEQ ID NO: 21 - DNA, 1716 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgagatacgacgttgttatcgctggtgctggtccaactggtttgatgttggcttgtgaattgagattggctggtgctagaactttggttttggaaagattggctgaaccagttgacttctctaaggctttgggtgttcacgctagaactgttgaattgttggacatgagaggtttgggtgaaggtttccaagctgaagctccaaagttgagaggtggtaacttcgcttctttgggtgttccattggacttctcttctttcgacactagacacccatacgctttgttcgttccacaagttagaactgaagaattgttgactggtagagctttggaattgggtgctgaattgagaagaggtcacgctgttactgctttggaacaagacgctgacggtgttactgtttctgttactggtccagaaggtccatacgaagttgaatgtgcttacttggttggttgtgacggtggtggttctactgttagaaagttgttgggtatcgacttcccaggtcaagacccacacatgttcgctgttatcgctgacgctagattcagagaagaattgccacacggtgaaggtatgggtccaatgaggccatacggtgttatgagacacgacttgagagcttggttcgctgctttcccattggaaccagacgtttacagagctactgttgctttcttcgacagaccatacgctgacagaagagcgccagttactgaagaagatgttagagctgctttgactgaagttgctggttctgacttcggtatgcacgacgttagatggttgtctcgtttgactgacacttctcgtcaagctgaaagatacagagatggtagagttttgttggctggtgacgcttgtcacatccacttgccagctggtggtcaaggtttgaacttgggtttccaagacgctgttaacttgggttggaagttgggtgctactatcgctggtactgctccaccagaattgttggacacttacgaagctgaaagaaggccaatcgctgctggtgttttgagaaacactagagcgcaagctgttttgatcgacccagacccaagatacgaaggtttgagagaattgatgatcgaattgttgcacgttccagaaactaacagatacttggctggtttgatctctgctttggacgttagatacccaatggctggtgaacacccattgttgggtagaagagttccagacttgccattggttactgaagatggtactagacaattgtctacttacttccacgctgctagaggtgttttgttgactttgggttgtgaccaaccattggctgacgaagctgctgcttggaaggacagagttgacttggttgctgctgaaggtgttgctgacccaggttctgctgttgacggattaactgctttgttggttagaccagacggttacatctgttggactgctgctccagaaactggtactgacggtttgactgacgctttgagaacttggttcggtccacctgctatggtatcgatggattacaaggatgacgacgataagatctgagctcttaattaacaattcttcgccagaggtttggtcaagtctccaatcaaggttgtcggct

SEQ ID NO: 22 - DNA, 1689 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgttggttgctggtggtggtccaactggtttgtggttggctggtgaattgagattggctggtgttagaccattggttttggaaagaagaactgaaccattcccacactctaaggctttgggtatccacccaagaactatcgaaatgttggaattgagaggtttggctggtagattcttggacggtgctccaaagttgccaaagggtcacttcgcttctttgccagttccattggactacggtgctatggacactagacacccatacggtgtttacagaaagcaagttgaaactgaagctttgttggctggtcacgctactagattgggtgttccagttagaagaggtcacgaattggttggtttgagacaacacgacgacgctgttgttggtactgttcaaggtccacaaggtagatacgaagttagagctgcttacttggttggttgtgacggtggtggttctactactagaagattggctggtatcgacttcccaggtcaagacccaggtgttgctgttttgatggctgacgctagattcagagatccattgccatctgacccagctatgggtccattcagaagatacggtgttttaagaccagacttgagagcttggttctctgctatcccattgccagaagaacaaggtttgtgtagagttatggttttctggtacggtagacaattcgaagatagaagagcgccagttactgaagatgaaatgagagctgctttggttgacttggctggtactgacttcggtatgtacgacgttcaatggttgactagattcactgacgcttctcgtcaagctgctagatacagatcgggtagagttttcttggctggtgacgctgctcacactgttttcccaactggtggtccaggtttgaacgctggtttgcaagacgctatgaacttgggttggaagttggctggtgctgttcacggttggggtggtgctttgttggactcttaccacgacgaaagatacccagttgctgaacaagttttgagagacactagaactcaatgtttgttgttggacccagacccaagattcgttccattgagagaaactttggctgaattgttgagattggaaccagttaacagatacttcactggtcacaacactgctttggctgttagatacgacttgggtggtgctcacccagctactggtggtagaatgccagacgttttggttgctactggtgctggtcaaagaagattctctgactgttgtgttactggtagaggtgttttgttggttacttctggtgctgacagaggtgaattgttgagaggttggaaggacagagttgacgttgttactggtactgttgctggtccattggacgctgctgaccacttggttagaccagacggttacgttgcttgggctggtggtgctggtcacgacgaaccaggtttggaaactgctttgagattgtggttcggtgaaccagtttctgtatcgatggattacaaggatgacgacgataagatctgagctcttaattaacaattcttcgccagaggtttggtcaagtctccaatcaaggttgtcggct

SEQ ID NO: 23 - DNA, 1614 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatggttgttgctggtgctggtccaactggtttgatgttggcttgtgaattggctttgggtggtgctagagctgttgttgttgaaagaagaagagaaccagaaaagcactctaaggctatgggtatgcaaggtagaactgttgaattgttggaattgagaggtttgttggacagattcaaggaaggtgctggtgttttgcaaggtggtaacttcgcttctttgggtgttccaatgagattcgaagaatttgacactcaaccaactcaatacgttttgttggttccacaattgagaactgaagaattgttggctgaaagagctagagaattgggtgttagaatcgttagaggttctggtgttactggtttcgctcaagacgctgacggtgttactgttgaaactgacactggtttgttgagagctagatacttggttggttgtgacggtggttctactgttagaaaggctgctggtatcggtttcactggtcaagacccacacatgtacgctttgatcggtgacatgagattctctggtgacttgccaagaggtgaaggtttgggtccaatgaggccagttggtttggtttacagagctactgttgcttggttcgacagaccattcgctgacagaagagcgccagttactgaagaagaaatgagagctgctttggttgaacacactggttctgaccacggtatgcacgacgttacttggttgtctcgtttgactgacgtttctcgtttggctgactcttacagattgggtagagttttgttggctggtgacgctgctcacatccacttgccagctggtggtcaaggtttgaacttgggtttccaagacgctgttaacttgggttggaagttggctgctgttgttagaggtcacggtactgaagaattgttggactcttacggtagagaaagaaggccaatcgctgacggtgttgttagaaacactagaactcaagctgttttgatcgacccagacccaagatacgaagctctcagacaaactttgccacacttgatggctttgccagacactaacagacacatggctggtatgttgtctggtttcgacgttgcttacggtggtggtgaccacccattggttggtagaagaatgccagacgctgaattgatcactgctgacggtccaagaagaatctctgactgtttcgctggtgctagaggtttgttgttgttgccagaacaaggtccaactgcttctccattggctgcttgggctgacagagttgacactttgactgttaagtctggtggtccagacccagacactgctcacttggttagaccagacggttacgttgcttgggctggtgaaccagctagaactgaagaattgcaccacgctgctactacttggttcggtgctgctgctgtatcgatggattacaaggatgacgacgataagatctgagctcttaattaacaattcttcgccagaggtttggtcaagtctccaatcaaggttgtcggct

SEQ ID NO: 24 - DNA, 1680 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatggacgctgctgttatcgttgttggtgctggtccagctggtatgatgttggctggtgaattgagattggctggtgttgaagttgttgttttggaaagattggttgaaagaactggtgaatctcgtggtttgggtttcactgctagaactatggaagttttcgaccaaagaggtatcttgccaagattcggtgaagttgaaacttctactcaaggtcacttcggtggtttgccaatcgacttcggtgttttggaaggtgcttggcaagctgctaagactgttccacaatctgttactgaaactcacttggaacaatgggctactggtttgggtgctgacatcagaagaggtcacgaagttttgtctttgactgacgacggtgctggtgttactgttgaagttagaggtccagaaggtaagcacactttgagagctgcttacttggttggttgtgacggtggtagatcgtctgttagaaaggctgctggtttcgacttcccaggtactgctgctactatggaaatgtacttggctgacatcaagggtgttgaattgcaaccaagaatgatcggtgaaactttgccaggtggtatggttatggttggtccattgccaggtggtatcactagaatcatcgtttgtgaaagaggtactccaccacaaagaagagaaactccaccatcttggcacgaagttgctgacgcttggaagagattgactggtgacgacatcgctcacgctgaaccagtttgggtttctgctttcggtaacgctactagacaagttactgaatacagaagaggtagagttatcttggctggtgactctgctcacatccacttgccagctggtggtcaaggtatgaacacttctatccaagacgctgttaacttgggttggaagttgggtgctgttgttaacggtactgctactgaagaattgttggactcttaccactctgaaagacacgctgttggtaagagattgttgatgaacactcaagctcaaggtttgttgttcttgtctggtccagaagttcaaccattgagagatgttttgactgaattgatccaatacggtgaagttgctagacacttggctggtatggtttctggtttggaaatcacttacgacgttggtactggttctcacccattgttgggtaagagaatgccagctttggaattgactactgctactagagaaacatcttctactgaattattacacactgctagaggtgttttgttggacttggctgacaacccaagattgagagctagagctgctgcttggtctgacagagttgacatcgttactgctgttccaggtgaagtttctgctacttctggtttgagagacactactgctgttttgataagaccagacggtcacgttgcttgggctgctccaggttctcaccacgacttgccaatggctttggaaagatggttcggtgctccattgactggtgtatcgatggattacaaggatgacgacgataagatctgagctcttaattaacaattcttcgccagaggtttggtcaagtctccaatcaaggttgtcggct

SEQ ID NO: 25 - DNA, 1749 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatggaaccagaagttgttgttgctggtgctggtccagttggtttgatgttggcttgtgaattgagaaggcaaggtgttggtgttgttttggttgaaagaactactgaaccagctaacgacaacagagcgcaagctctccacggtagaactatcccagttttggacagaagaggtttgttgccaagattcagagctgctgaaagagaattgaacggtggtggttctggtggtactgaatctcacagaaaaagaccattgccaagaggtcacttcgctggtatcactggtttattaccatctgctccagacccagacccagacgttccaccagttgttttcgttccacaatgggttactactagattgttgactgaacacgctgctgaattgggtgttgttttgcacagaggttctgaaatcactgctgttgaacaagacgctgacggtgttactgttgctgtttctggtggtactgctccagctagattgagagctagatacttgataggttgtgacggtgctagatcggttgttagaagatcggctggtatcgacttcgctggtactggtgctactttgtctactttggctgctgaagttcacttggacgacccagctgacttgccagttggttggcacagaactcaacacggttgtactgttatctctccacacccagaaggtggtcactctcgtgttgttgttatcgacttctctggtccagacgctgacagagaagctccagttactttgactgaattggaagaaagaatcgctggtgttttgggtagaaaggttgctatgtctgacttgaagaacgctcaaagatacggtgacgctgctagattggctgacagatacagagttggtagagttttcttggctggtgacgctgctcacatccactacccagttggtggtcaaggtttgaacatcggtttgcaagacgctgttaacttggcttggaagttggctgctgacttgagaggttgggctccagacggtttgttggacacttaccacactgaaagatacccagttgctgactctgttttgactcacactagagcgcaagttgctttgttggctccaggtccaagaatcgacgctttgagaactttgttcactgacttgatcggtttggctgacgtttctcgttacttgtctgaaaagatgtctgctgctgacgttagatacgacatgggtgaagctgaaccacacccattgactggtagattcgctccatctttgagattggctactgaacaaggtgaaagaagattggctgacttgttggctggtggtagaccattgttgttggacttgtctggtagagctgctactgctgctgttgctgctagatgggctgacagagttgacgttcacgctgctacttgtccagctgaaccagctttgggttctttgttgatcagaccagactcttacgttgcttgggcttgtgctccaggtgacgctccagaccaagttgaaagaggtttggaagctgctttgagaagatggttcggtgctccagaccaccaagtatcgatggattacaaggatgacgacgataagatctgagctcttaattaacaattcttcgccagaggtttggtcaagtctccaatcaaggttgtcggct

SEQ ID NO: 26 - DNA, 1921 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgccattatgcagatcttcgtcaagacgttaaccggtaaaaccataactctagaagttgaatcttccgataccatcgacaacgttaagtcgaaaattcaagacaaggaaggcattccacctgatcaacaaagattgatctttgccggtaagcagctcgaggacggtagaacgctgtctgattacaacattcagaaggagtcgaccttacatcttgtcttaagactaagaggtggtatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 27 - DNA, 2146 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgccattatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 28 - DNA, 2547 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgactaaatctcattcagaagaagtgattgtacctgagttcaattctagcgcaaaggaattaccaagaccattggccgaaaagtgcccgagcataattaagaaatttataagcgcttatgatgctaaaccggattttgttgctagatcgcctggtagagtcaatctaattggtgaacatattgattattgtgacttctcggttttacctttagctattgattttgatatgctttgcgccgtcaaagttttgaacgagaaaaatccatccattaccttaataaatgctgatcccaaatttgctcaaaggaagttcgatttgccgttggacggttcttatgtcacaattgatccttctgtgtcggactggtctaattactttaaatgtggtctccatgttgctcactcttttctaaagaaacttgcaccggaaaggtttgccagtgctcctctggccgggctgcaagtcttctgtgagggtgatgtaccaactggcagtggattgtcttcttcggccgcattcatttgtgccgttgctttagctgttgttaaagcgaatatgggccctggttatcatatgtccaagcaaaatttaatgcgtattacggtcgttgcagaacattatgttggtgttaacaatggcggtatggatcaggctgcctctgtttgcggtgaggaagatcatgctctatacgttgagttcaaaccgcagttgaaggctactccgtttaaatttccgcaattaaaaaaccatgaaattagctttgttattgcgaacacccttgttgtatctaacaagtttgaaaccgccccaaccaactataatttaagagtggtagaagtcactacagaattccggatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 29 - DNA, 2314 bp, Artificial Sequence
  Description of Artificial Sequence Synthetic polynucleotide
ccctcactaaagggaacaaaagctggagctctagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggattctagaactagtatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgaaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 30 - DNA, 1927 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagttgagagctcttaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctccaattcagctggcgtaatagcgaaga

SEQ ID NO: 31 - DNA, 2432 bp, Artificial Sequence
  Description of Artificial Sequence Synthetic polynucleotide
ccctcactaaagggaacaaaagctggagctcgcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaataccatatggctatctagaactagtatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgaaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 32 - DNA, 2353 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgccattatgcagatcttcgtcaagacgttaaccggtaaaaccataactctagaagttgaatcttccgataccatcgacaacgttaagtcgaaaattcaagacaaggaaggcattccacctgatcaacaaagattgatctttgccggtaagcagctcgaggacggtagaacgctgtctgattacaacattcagaaggagtcgaccttacatcttgtcttaagactaagaggtggtatgcaagacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 33 - DNA, 2125 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgccattatgcaagacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 34 - DNA, 2607 bp, Artificial Sequence
  Description of Artificial Sequence Synthetic polynucleotide
tggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacgatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcgg

SEQ ID NO: 35 - DNA, 2607 bp, Artificial Sequence
  Description of Artificial Sequence Synthetic polynucleotide
tggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtatgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacggtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcgg

SEQ ID NO: 36 - DNA, 1701 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaggtacgatgttgttatagctggtgcaggacccaccggtttgatgttagcatgtgaacttcggctggcgggtgccagaactttggttttagaaagattagccgagccttttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 37 - DNA, 1701 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 38 - DNA, 1701 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactagacacccatatgcattgtttgttccacaagtacgaactgaagaactgctaacaggtagagctttggagctaggggcggagctgcgtcgtggtcatgccgtgaccgccttggaacaagatgctgatggtgttactgtgagcgtgacaggccctgaaggcccatacgaagtagaatgtgcttatttggtgggctgcgacggtggaggcagtacggtgaggaaactattgggcatagattttccaggtcaagacccacatatgtttgctgtcatcgcagacgcaagattcagagaagagcttcctcacgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtgtatcgatggattacaaggatgacgacgataagatctgagctcttaattaacaattcttcgccagaggtttggtcaagtctccaatcaaggttgtcggct

SEQ ID NO: 39 - DNA, 1299 bp, Artificial Sequence
  Description of Artificial Sequence Synthetic polynucleotide
tggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgaccgcaggacagctgcatcttgacgttccggaaccgcctggttcatgggtagatgaagccacatttagggctgttatgggtgctttgccatccggtgttacagtcgtgacgaccttggacccagatggcagaccagtaggcttaacttgttctgcggcttgttcagtttctcaaaggcctcccttattacttgtctgcttaaacgatagaagcagggtactagatgcaatactcagaagaggtcagttcatagtaaatgtcctacgcgaccgtagagaagcaacatcggcagtctttgccagtagagccgaagatagattcgcagctgtgccttggcaacctgggaggcgaactggtgtcccttggttgttagcagacacggttgctcatgcagaatgtgaggtggcaggtactttgcatgctggcgatcacgttatcgttgttggaggtattgttgcgggtgatgcgcaccccgaccgtgtggggccattaatgtattggagacaaagatacggtggatggccagtaacagatgatgctagagaaattgcattgactctagctgccgagggagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcgg

SEQ ID NO: 40 - DNA, 2019 bp, Artificial Sequence
  Description of Artificial Sequence Synthetic polynucleotide
ccctcactaaagggaacaaaagctggagctcagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgactgatgtatccgcccatactacaaccgatggggcggttgcaagggctcgcgctgtggtcagactaaatactgcatattttcgggctaaggtacttcaaagtgctgtcgaactaggtgtttttgatcttttagatggaggcccacaacctgccgctgacatttgcggaaagttaggtattagacatagactcgcagtggactatttggacgcattgacgggtttagggttgcttgaaagggatggtgaacgttacagaaactcgccaacagcggctgagttcttggtccaagatcgacccgcctatttgggtggaaccgcgaggcaacatgccaaattacattatcacgcttggggtaaactggccgatgcaatgagagaaggccgtgctcagagcgccgtggcagctcaaggtgcaaaagcctatgtaaattactatgaagatttgggccaggccaggagggtcatgacacatatggatgctcataacggtttcacagcggatgaaatcgctagacaattggactggagaggatacgccacagtaacagatgtgggcggcgctcgtgggaatgttgcagctcgaattgttcaagcagtgcctcatttgagagcaaatgtagttgacctacccgcattgcgacctttattcgatgagctgatggcccaactaggaactgctggtcaggttgagtttcacggtggtgacttctttgcggacccgataccagagtctgacgttatcgttgtgggccacgtcctggcggattggccacagccaagacgtgtggaattacttagaagatgtcacgcagcattaagacctggtggaacggtagttgtttacgatgttatggttgatgatgatagagatgacgtcgaagcactcctacaaagattaaactctgcaatgataagggacgatatgggtgcttacgctgttactgaatgtgctggatatttaagagaagctggatttaccgtagaaagggccctgcgtacggataccataactcgcgatcattttgttattggtagaaaagctgcatcataactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaat

SEQ ID NO: 41 - DNA, 1483 bp, Artificial Sequence
  Description of Artificial Sequence Synthetic polynucleotide
atgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagta

SEQ ID NO: 42 - DNA, 402 bp, Artificial Sequence
  Description of Artificial Sequence Synthetic polynucleotide
atgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacg

SEQ ID NO: 43 - DNA, 1509 bp, Artificial Sequence
  Description of Artificial Sequence Synthetic polynucleotide
atgagatacgacgttgttatcgctggtgctggtccaactggtttgatgttggcttgtgaattgagattggctggtgctagaactttggttttggaaagattggctgaaccagttgacttctctaaggctttgggtgttcacgctagaactgttgaattgttggacatgagaggtttgggtgaaggtttccaagctgaagctccaaagttgagaggtggtaacttcgcttctttgggtgttccattggacttctcttctttcgacactagacacccatacgctttgttcgttccacaagttagaactgaagaattgttgactggtagagctttggaattgggtgctgaattgagaagaggtcacgctgttactgctttggaacaagacgctgacggtgttactgtttctgttactggtccagaaggtccatacgaagttgaatgtgcttacttggttggttgtgacggtggtggttctactgttagaaagttgttgggtatcgacttcccaggtcaagacccacacatgttcgctgttatcgctgacgctagattcagagaagaattgccacacggtgaaggtatgggtccaatgaggccatacggtgttatgagacacgacttgagagcttggttcgctgctttcccattggaaccagacgtttacagagctactgttgctttcttcgacagaccatacgctgacagaagagcgccagttactgaagaagatgttagagctgctttgactgaagttgctggttctgacttcggtatgcacgacgttagatggttgtctcgtttgactgacacttctcgtcaagctgaaagatacagagatggtagagttttgttggctggtgacgcttgtcacatccacttgccagctggtggtcaaggtttgaacttgggtttccaagacgctgttaacttgggttggaagttgggtgctactatcgctggtactgctccaccagaattgttggacacttacgaagctgaaagaaggccaatcgctgctggtgttttgagaaacactagagcgcaagctgttttgatcgacccagacccaagatacgaaggtttgagagaattgatgatcgaattgttgcacgttccagaaactaacagatacttggctggtttgatctctgctttggacgttagatacccaatggctggtgaacacccattgttgggtagaagagttccagacttgccattggttactgaagatggtactagacaattgtctacttacttccacgctgctagaggtgttttgttgactttgggttgtgaccaaccattggctgacgaagctgctgcttggaaggacagagttgacttggttgctgctgaaggtgttgctgacccaggttctgctgttgacggattaactgctttgttggttagaccagacggttacatctgttggactgctgctccagaaactggtactgacggtttgactgacgctttgagaacttggttcggtccacctgctatg

SEQ ID NO: 44 - DNA, 1482 bp, Artificial Sequence
  Description of Artificial Sequence Synthetic polynucleotide
atgttggttgctggtggtggtccaactggtttgtggttggctggtgaattgagattggctggtgttagaccattggttttggaaagaagaactgaaccattcccacactctaaggctttgggtatccacccaagaactatcgaaatgttggaattgagaggtttggctggtagattcttggacggtgctccaaagttgccaaagggtcacttcgcttctttgccagttccattggactacggtgctatggacactagacacccatacggtgtttacagaaagcaagttgaaactgaagctttgttggctggtcacgctactagattgggtgttccagttagaagaggtcacgaattggttggtttgagacaacacgacgacgctgttgttggtactgttcaaggtccacaaggtagatacgaagttagagctgcttacttggttggttgtgacggtggtggttctactactagaagattggctggtatcgacttcccaggtcaagacccaggtgttgctgttttgatggctgacgctagattcagagatccattgccatctgacccagctatgggtccattcagaagatacggtgttttaagaccagacttgagagcttggttctctgctatcccattgccagaagaacaaggtttgtgtagagttatggttttctggtacggtagacaattcgaagatagaagagcgccagttactgaagatgaaatgagagctgctttggttgacttggctggtactgacttcggtatgtacgacgttcaatggttgactagattcactgacgcttctcgtcaagctgctagatacagatcgggtagagttttcttggctggtgacgctgctcacactgttttcccaactggtggtccaggtttgaacgctggtttgcaagacgctatgaacttgggttggaagttggctggtgctgttcacggttggggtggtgctttgttggactcttaccacgacgaaagatacccagttgctgaacaagttttgagagacactagaactcaatgtttgttgttggacccagacccaagattcgttccattgagagaaactttggctgaattgttgagattggaaccagttaacagatacttcactggtcacaacactgctttggctgttagatacgacttgggtggtgctcacccagctactggtggtagaatgccagacgttttggttgctactggtgctggtcaaagaagattctctgactgttgtgttactggtagaggtgttttgttggttacttctggtgctgacagaggtgaattgttgagaggttggaaggacagagttgacgttgttactggtactgttgctggtccattggacgctgctgaccacttggttagaccagacggttacgttgcttgggctggtggtgctggtcacgacgaaccaggtttggaaactgctttgagattgtggttcggtgaaccagtttct

SEQ ID NO: 45 - DNA, 1407 bp, Artificial Sequence
  Description of Artificial Sequence Synthetic polynucleotide
atggttgttgctggtgctggtccaactggtttgatgttggcttgtgaattggctttgggtggtgctagagctgttgttgttgaaagaagaagagaaccagaaaagcactctaaggctatgggtatgcaaggtagaactgttgaattgttggaattgagaggtttgttggacagattcaaggaaggtgctggtgttttgcaaggtggtaacttcgcttctttgggtgttccaatgagattcgaagaatttgacactcaaccaactcaatacgttttgttggttccacaattgagaactgaagaattgttggctgaaagagctagagaattgggtgttagaatcgttagaggttctggtgttactggtttcgctcaagacgctgacggtgttactgttgaaactgacactggtttgttgagagctagatacttggttggttgtgacggtggttctactgttagaaaggctgctggtatcggtttcactggtcaagacccacacatgtacgctttgatcggtgacatgagattctctggtgacttgccaagaggtgaaggtttgggtccaatgaggccagttggtttggtttacagagctactgttgcttggttcgacagaccattcgctgacagaagagcgccagttactgaagaagaaatgagagctgctttggttgaacacactggttctgaccacggtatgcacgacgttacttggttgtctcgtttgactgacgtttctcgtttggctgactcttacagattgggtagagttttgttggctggtgacgctgctcacatccacttgccagctggtggtcaaggtttgaacttgggtttccaagacgctgttaacttgggttggaagttggctgctgttgttagaggtcacggtactgaagaattgttggactcttacggtagagaaagaaggccaatcgctgacggtgttgttagaaacactagaactcaagctgttttgatcgacccagacccaagatacgaagctctcagacaaactttgccacacttgatggctttgccagacactaacagacacatggctggtatgttgtctggtttcgacgttgcttacggtggtggtgaccacccattggttggtagaagaatgccagacgctgaattgatcactgctgacggtccaagaagaatctctgactgtttcgctggtgctagaggtttgttgttgttgccagaacaaggtccaactgcttctccattggctgcttgggctgacagagttgacactttgactgttaagtctggtggtccagacccagacactgctcacttggttagaccagacggttacgttgcttgggctggtgaaccagctagaactgaagaattgcaccacgctgctactacttggttcggtgctgctgct

SEQ ID NO: 46 - DNA, 1473 bp, Artificial Sequence
  Description of Artificial Sequence Synthetic polynucleotide
atggacgctgctgttatcgttgttggtgctggtccagctggtatgatgttggctggtgaattgagattggctggtgttgaagttgttgttttggaaagattggttgaaagaactggtgaatctcgtggtttgggtttcactgctagaactatggaagttttcgaccaaagaggtatcttgccaagattcggtgaagttgaaacttctactcaaggtcacttcggtggtttgccaatcgacttcggtgttttggaaggtgcttggcaagctgctaagactgttccacaatctgttactgaaactcacttggaacaatgggctactggtttgggtgctgacatcagaagaggtcacgaagttttgtctttgactgacgacggtgctggtgttactgttgaagttagaggtccagaaggtaagcacactttgagagctgcttacttggttggttgtgacggtggtagatcgtctgttagaaaggctgctggtttcgacttcccaggtactgctgctactatggaaatgtacttggctgacatcaagggtgttgaattgcaaccaagaatgatcggtgaaactttgccaggtggtatggttatggttggtccattgccaggtggtatcactagaatcatcgtttgtgaaagaggtactccaccacaaagaagagaaactccaccatcttggcacgaagttgctgacgcttggaagagattgactggtgacgacatcgctcacgctgaaccagtttgggtttctgctttcggtaacgctactagacaagttactgaatacagaagaggtagagttatcttggctggtgactctgctcacatccacttgccagctggtggtcaaggtatgaacacttctatccaagacgctgttaacttgggttggaagttgggtgctgttgttaacggtactgctactgaagaattgttggactcttaccactctgaaagacacgctgttggtaagagattgttgatgaacactcaagctcaaggtttgttgttcttgtctggtccagaagttcaaccattgagagatgttttgactgaattgatccaatacggtgaagttgctagacacttggctggtatggtttctggtttggaaatcacttacgacgttggtactggttctcacccattgttgggtaagagaatgccagctttggaattgactactgctactagagaaacatcttctactgaattattacacactgctagaggtgttttgttggacttggctgacaacccaagattgagagctagagctgctgcttggtctgacagagttgacatcgttactgctgttccaggtgaagtttctgctacttctggtttgagagacactactgctgttttgataagaccagacggtcacgttgcttgggctgctccaggttctcaccacgacttgccaatggctttggaaagatggttcggtgctccattgactggt

SEQ ID NO: 47 - DNA, 1542 bp, Artificial Sequence
  Description of Artificial Sequence Synthetic polynucleotide
atggaaccagaagttgttgttgctggtgctggtccagttggtttgatgttggcttgtgaattgagaaggcaaggtgttggtgttgttttggttgaaagaactactgaaccagctaacgacaacagagcgcaagctctccacggtagaactatcccagttttggacagaagaggtttgttgccaagattcagagctgctgaaagagaattgaacggtggtggttctggtggtactgaatctcacagaaaaagaccattgccaagaggtcacttcgctggtatcactggtttattaccatctgctccagacccagacccagacgttccaccagttgttttcgttccacaatgggttactactagattgttgactgaacacgctgctgaattgggtgttgttttgcacagaggttctgaaatcactgctgttgaacaagacgctgacggtgttactgttgctgtttctggtggtactgctccagctagattgagagctagatacttgataggttgtgacggtgctagatcggttgttagaagatcggctggtatcgacttcgctggtactggtgctactttgtctactttggctgctgaagttcacttggacgacccagctgacttgccagttggttggcacagaactcaacacggttgtactgttatctctccacacccagaaggtggtcactctcgtgttgttgttatcgacttctctggtccagacgctgacagagaagctccagttactttgactgaattggaagaaagaatcgctggtgttttgggtagaaaggttgctatgtctgacttgaagaacgctcaaagatacggtgacgctgctagattggctgacagatacagagttggtagagttttcttggctggtgacgctgctcacatccactacccagttggtggtcaaggtttgaacatcggtttgcaagacgctgttaacttggcttggaagttggctgctgacttgagaggttgggctccagacggtttgttggacacttaccacactgaaagatacccagttgctgactctgttttgactcacactagagcgcaagttgctttgttggctccaggtccaagaatcgacgctttgagaactttgttcactgacttgatcggtttggctgacgtttctcgttacttgtctgaaaagatgtctgctgctgacgttagatacgacatgggtgaagctgaaccacacccattgactggtagattcgctccatctttgagattggctactgaacaaggtgaaagaagattggctgacttgttggctggtggtagaccattgttgttggacttgtctggtagagctgctactgctgctgttgctgctagatgggctgacagagttgacgttcacgctgctacttgtccagctgaaccagctttgggttctttgttgatcagaccagactcttacgttgcttgggcttgtgctccaggtgacgctccagaccaagttgaaagaggtttggaagctgctttgagaagatggttcggtgctccagaccaccaa

SEQ ID NO: 48 - DNA, 1710 bp, Artificial Sequence
  Description of Artificial Sequence Synthetic polynucleotide
atgcagatcttcgtcaagacgttaaccggtaaaaccataactctagaagttgaatcttccgataccatcgacaacgttaagtcgaaaattcaagacaaggaaggcattccacctgatcaacaaagattgatctttgccggtaagcagctcgaggacggtagaacgctgtctgattacaacattcagaaggagtcgaccttacatcttgtcttaagactaagaggtggtatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 49 - DNA, 1935 bp, Artificial Sequence
  Description of Artificial Sequence Synthetic polynucleotide
atggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 50 - DNA, 2332 bp, Artificial Sequence
  Description of Artificial Sequence Synthetic polynucleotide
atgactaaatctcattcagaagaagtgattgtacctgagttcaattctagcgcaaaggaattaccaagaccattggccgaaaagtgcccgagcataattaagaaatttataagcgcttatgatgctaaaccggattttgttgctagatcgcctggtagagtcaatctaattggtgaacatattgattattgtgacttctcggttttacctttagctattgattttgatatgctttgcgccgtcaaagttttgaacgagaaaaatccatccattaccttaataaatgctgatcccaaatttgctcaaaggaagttcgatttgccgttggacggttcttatgtcacaattgatccttctgtgtcggactggtctaattactttaaatgtggtctccatgttgctcactcttttctaaagaaacttgcaccggaaaggtttgccagtgctcctctggccgggctgcaagtcttctgtgagggtgatgtaccaactggcagtggattgtcttcttcggccgcattcatttgtgccgttgctttagctgttgttaaagcgaatatgggccctggttatcatatgtccaagcaaaatttaatgcgtattacggtcgttgcagaacattatgttggtgttaacaatggcggtatggatcaggctgcctctgtttgcggtgaggaagatcatgctctatacgttgagttcaaaccgcagttgaaggctactccgtttaaatttccgcaattaaaaaaccatgaaattagctttgttattgcgaacacccttgttgtatctaacaagtttgaaaccgccccaaccaactataatttaagagtggtagaagtcactacagatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 51 - DNA, 1482 bp, Artificial Sequence
  Description of Artificial Sequence Synthetic polynucleotide
atgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 52 - DNA, 1486 bp, Artificial Sequence
  Description of Artificial Sequence Synthetic polynucleotide
catgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagttga

SEQ ID NO: 53 - DNA, 2061 bp, Artificial Sequence
  Description of Artificial Sequence Synthetic polynucleotide
gcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaataccatatggctaatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 54 - DNA, 2142 bp, Artificial Sequence
  Description of Artificial Sequence Synthetic polynucleotide
atgcagatcttcgtcaagacgttaaccggtaaaaccataactctagaagttgaatcttccgataccatcgacaacgttaagtcgaaaattcaagacaaggaaggcattccacctgatcaacaaagattgatctttgccggtaagcagctcgaggacggtagaacgctgtctgattacaacattcagaaggagtcgaccttacatcttgtcttaagactaagaggtggtatgcaagacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 55 - DNA, 1914 bp, Artificial Sequence
  Description of Artificial Sequence Synthetic polynucleotide
atgcaagacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 56 - DNA, 1884 bp, Artificial Sequence
  Description of Artificial Sequence Synthetic polynucleotide
atgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacgatgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 57 - DNA, 1884 bp, Artificial Sequence
  Description of Artificial Sequence Synthetic polynucleotide
atgttagtagccggtggtggaccaacagggttatggttggctggtgaactaaggttggccggggttagacctcttgttttagagcgtaggacagaaccctttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagtatgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacg

SEQ ID NO: 58 - DNA, 1494 bp, Artificial Sequence
  Description of Artificial Sequence Synthetic polynucleotide
atgaggtacgatgttgttatagctggtgcaggacccaccggtttgatgttagcatgtgaacttcggctggcgggtgccagaactttggttttagaaagattagccgagccttttccacatagcaaagcactgggtatccatccaagaactattgaaatgttggagttgagaggccttgctggtagatttttagatggcgccccgaagctgccgaaaggtcactttgcatcgcttcctgtgcccttagactatggtgcaatggatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 59 - DNA, 426 bp, Artificial Sequence
  Description of Artificial Sequence Synthetic polynucleotide
atgccattcactcaaaaggagatcacgtatttaagggctcaaggctacggccgactagccaccgtcggtgctcacggtgaacctcacaacgtgccggtatcttttgaaattgatgaagaaagaggtaccattgaaataacaggaagggatatgggacgttccagaaaattcagaaatgttgccaaaagtgacagagtggcatttgttgttgatgatgttccatgcagagacccagaagttgtcagagctgttgtaatacatggtactgcacaggcgcttcccacaggcggaagagaaaggcgtcctcattgtgctgacgaaatgattagaattcatccaagaagaatagtaacatggggtatcgaaggtgatttgtcaactggtgttcatgcaagagatattactgctgaagatggtggtagaagg

SEQ ID NO: 60 - DNA, 1494 bp, Artificial Sequence
  Description of Artificial Sequence Synthetic polynucleotide
atgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactcgacatccgtacggtgtctaccgtaaacaagtggaaacggaagctttattagccggccacgcaaccagattaggagttccagtccgcagaggtcatgaacttgtaggcctaagacagcacgatgatgctgtagtcggtactgttcaaggtcctcaaggtagatacgaagtcagagccgcttatcttgttggttgtgatggaggaggttctaccacaagacgcctggctgggatagatttccctggacaagatcctggtgttgccgttttgatggctgacgcaagattccgggacccattaccctctgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 61 - DNA, 1494 bp, Artificial Sequence
  Description of Artificial Sequence Synthetic polynucleotide
atgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactagacacccatatgcattgtttgttccacaagtacgaactgaagaactgctaacaggtagagctttggagctaggggcggagctgcgtcgtggtcatgccgtgaccgccttggaacaagatgctgatggtgttactgtgagcgtgacaggccctgaaggcccatacgaagtagaatgtgcttatttggtgggctgcgacggtggaggcagtacggtgaggaaactattgggcatagattttccaggtcaagacccacatatgtttgctgtcatcgcagacgcaagattcagagaagagcttcctcacgatccagctatgggtccattcagacgctatggagtcctgagacctgatttaagggcatggtttagtgctattcctttaccagaagaacagggtttatgcagagttatggttttttggtatggacgacaatttgaagatagacgtgctccagtaactgaggatgaaatgagagcagcacttgttgatctagcgggaactgactttggtatgtacgatgttcaatggttgaccagattcacagacgcttccaggcaagctgcaaggtatagatctggaagggttttcttagctggagatgctgctcatactgtcttccccactggtggcccaggtttgaatgcaggtttacaagatgccatgaatttaggttggaagttagctggagctgttcatggctggggtggtgctttactagattcatatcatgatgagcgttaccctgtagctgaacaagttttgcgtgacacaagaacacaatgtctattgttagatccagaccctcgctttgttccattaagagagacgttagcagaactattgagactagaacccgtaaacagatatttcaccggacacaacacggctctagcagttagatacgatttgggtggtgcccatcctgcaacgggaggcagaatgccagatgtcttggtcgcaactggtgctggtcaaagaaggttttcagattgttgtgttaccgggagaggggttcttttagtcacttctggtgcagataggggggaactgttacgaggatggaaagatagagtggatgtggtaacaggtaccgtggcaggacctcttgatgcagctgatcatttagttagacctgatggttatgttgcatgggccggtggagctggacacgatgaacctggtttagaaactgctcttagattatggtttggtgaaccagttagt

SEQ ID NO: 62 - DNA, 576 bp, Artificial Sequence
  Description of Artificial Sequence Synthetic polynucleotide
atgaccgcaggacagctgcatcttgacgttccggaaccgcctggttcatgggtagatgaagccacatttagggctgttatgggtgctttgccatccggtgttacagtcgtgacgaccttggacccagatggcagaccagtaggcttaacttgttctgcggcttgttcagtttctcaaaggcctcccttattacttgtctgcttaaacgatagaagcagggtactagatgcaatactcagaagaggtcagttcatagtaaatgtcctacgcgaccgtagagaagcaacatcggcagtctttgccagtagagccgaagatagattcgcagctgtgccttggcaacctgggaggcgaactggtgtcccttggttgttagcagacacggttgctcatgcagaatgtgaggtggcaggtactttgcatgctggcgatcacgttatcgttgttggaggtattgttgcgggtgatgcgcaccccgaccgtgtggggccattaatgtattggagacaaagatacggtggatggccagtaacagatgatgctagagaaattgcattgactctagctgccgaggga

SEQ ID NO: 63 - DNA, 1726 bp, Artificial Sequence
  Description of Artificial Sequence Synthetic polynucleotide
agtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaaatgactgatgtatccgcccatactacaaccgatggggcggttgcaagggctcgcgctgtggtcagactaaatactgcatattttcgggctaaggtacttcaaagtgctgtcgaactaggtgtttttgatcttttagatggaggcccacaacctgccgctgacatttgcggaaagttaggtattagacatagactcgcagtggactatttggacgcattgacgggtttagggttgcttgaaagggatggtgaacgttacagaaactcgccaacagcggctgagttcttggtccaagatcgacccgcctatttgggtggaaccgcgaggcaacatgccaaattacattatcacgcttggggtaaactggccgatgcaatgagagaaggccgtgctcagagcgccgtggcagctcaaggtgcaaaagcctatgtaaattactatgaagatttgggccaggccaggagggtcatgacacatatggatgctcataacggtttcacagcggatgaaatcgctagacaattggactggagaggatacgccacagtaacagatgtgggcggcgctcgtgggaatgttgcagctcgaattgttcaagcagtgcctcatttgagagcaaatgtagttgacctacccgcattgcgacctttattcgatgagctgatggcccaactaggaactgctggtcaggttgagtttcacggtggtgacttctttgcggacccgataccagagtctgacgttatcgttgtgggccacgtcctggcggattggccacagccaagacgtgtggaattacttagaagatgtcacgcagcattaagacctggtggaacggtagttgtttacgatgttatggttgatgatgatagagatgacgtcgaagcactcctacaaagattaaactctgcaatgataagggacgatatgggtgcttacgctgttactgaatgtgctggatatttaagagaagctggatttaccgtagaaagggccctgcgtacggataccataactcgcgatcattttgttattggtagaaaagctgcatcataactcgag

SEQ ID NO: 64 - DNA, 1905 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgatgggtgcaaaacacgttattagaagagtgaccaattcggaaaatgatacaggatcaagttcgaaatccactacttcatctaattccgacagggaccataatccctcttcctctaatagagacagatatcacaatgaaacatcacctgccaatatgatattgataaacgagccacctcctaccacctccacagaattggagagcgacactcatattaacgtacgtagcgtttctccatgtttggaaactggttccctaaaagcagagcaagcgcctcagaatgtccagaacccaccacagacacaggaccaagctactgaagatactgccaccaactctcaaccggcctcaacgcagttcagagctatcattgtgggaggtggcccaaacggtttgtgtctggcacatgcgttatacctagctggtattgattatgttttgctagagcgcggtggtgaaatcatcaaccaatctggagccggacttgctttatggcctcattcagttcgtatattagatcaattgggtctgctagacgaagctaggaaacattatttcccgattaagacaaagcataaccaccgccccgacgggtcagtcagagatgttaacgatatgtttgccaaggtagaagttaatcacggtcacccctggatgctgttccatcgagtaacattgctagaactcttgtgggaaaatttgcctgataaagagactagagtaaaagtcaacaagaaggtggaaagtgtagtctccaaccatgatggcgtcgtggttacatgttcagatggaacatctgaagcaggaagtatagtgataggttgcgatggtgtacactcgactgttagacaagtcatgcatgacttgagaagcgagaaaaagaagaaaaggccaggccgaaagctctctctgggaaggtcgccgtcacaagacaagccaatgagagcccactactatgggcttattggttggattccgcttcttgatgggttacaacctgctgcttgctacgaagtgagaagtgagcctaaaggtaaaacctttaccattttaacgggcgaagataccgcttacttcatcgtgtatattcatttagaaaaacccacgagagagcgcagcaggcacacagacgaggatgccgaaaggttggcagccgcattggccgagaacaagataacgagggaaattacttttggtgacctatggagatcaaggcgttggggcaaaatgcttgattttcaagaagggttcgtagataaatggtatcatgaaagaatcgttttagttggcgatgctgtccataagatgactccaaatgcgggactaggcttgaatgctggctggcaaggtatcgctgaattaacgaacagattgcggagattagttgttgcagaaggacaaagaccagatgcaaggtgcgttgaaaaagtttttcgaggttaccaagatagtagaaagggtatggcaaagaaaactatgaggttttcttctctgtacacccgtgtggtagctaatcagagcttactgtatcgtttttgtgatagaatgacaccagcggttggtggggatgtcgcattattaaatacaatggctagtcctattgttaaaaaaggtgtcactttcgactttgtagcggaacgtgatcataaagaaggtagagtaagatgggttcatccacaacatgttccagctgaagtatcgatggattacaaggatgacgacgataagatctgagctcttaattaacaattcttcgccagaggtttggtcaagtctccaatcaaggttgtcggct

SEQ ID NO: 65 - DNA, 1779 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaccaagtccaagttatacgacgtgataatcgcgggcgccggacccgtcggcctcttcctcgcccacgaactcagtctccgccaaatagcagtcctagtcctagaacgaacaacccaacaagactcgccctggaaatccggacctctaggcctccgaggcctaaacatccaatccatcgaagcattctatcgccgcggtttactaggccaattattcaatctggacgaaaggcaaaagtaccaaccgagcaagaaaccaggtttccaattcggcggccacttcgccggcatcataataaatgccaatcaattcgatttgcaacgctggaagtaccgcctctcaggtccggcgcttggtccgggtccaacgacgatggatcaagtggagaaggtcctaacggcgcgggtggagagtctaggcgtgaagattctccgcggtcgatctgttagccgtatctcgcaggatgaggcaggcgttacagtcgaaacagacgatgaccattccgagtcatttcggggcaaatggctcgttggctgtgacggtgggcgcagcgttgttcgaaaagaagccggcttcgaattcgtcggtacagatgcccaattcaccggctacgcaacgcatgtcgagatagaaggtcgcgagaagttgaagcctggtctcaacgtttctgaaacggggatgtatatcaacgtagtgcagaatggcgcttttcatctccttgaatttgacggggcgaacttcgatcgcgcgggtgatatcacgatcgatcaccttcagcgggtccttgatcgggctattggtcggacagatgtgaagattacaaatgtgcatcttgcttcttgttataccgatcgttcaatgcaggcggcaagttaccggaggaaccgcgttcttcttgccggtgatgcagcgcatattcattctccgctcggcgggcaaggactgaatgtcggactaggtgatgcgatgaatttggggtggaagcttgcggctatagttcagcgggaccaaggccagggagaggattctgggaatacgcatgatctgactcttctcgatacctacgagagcgagagacaccccattgctgcgtcggttcttgattggactcgtgcgcaggtcacggcgctgaggccggatgcctttggtcgggctacgcgcgccctgacggaagatctgatcaagacggatgatggggctaatctgtttattgatcggatctggggactttcgcagcgatataaacttggcgaagagtcgaggcctacacatccgcttcttgggtctagtgcgcctgatttcgagtttgggaatgggtctaggcttggctcgaagcttgaggagggacgggggttacttcttgattttgggaacagtagtgaactggaatcagttgttgatagaaaatattctgggaaggtagattatcttgctccagaggcaaaggagaactgtgggctgagtgccttgttgattcgacctgatggaatcgttgcatgggtggtggaagaagatgcgcagcacgatattgatgctttgaaagctgcattaggggattggttcactttggtatcgatggattacaaggatgacgacgataagatctgagctcttaattaacaattcttcgccagaggtttggtcaagtctccaatcaaggttgtcggct

SEQ ID NO: 66 - DNA, 1890 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgacaactaatccagaagtcatcatcgtgggcggcggagcagtcggcctggccgctgcatgcgaattggccatcaagaatgtctctgttacgatcttggagcgggaatctggacctgcagcgccatggaagaacgagaagctgggctttcgcgagctgtttgtgcctgccatcgagcacttgtaccgccgtggcctgctgaacgacatgttccgcaatgaagaacgaccacagactatgccggagaagaagggtggctttgagtttgctggtcattttgctggtatgatgatcaatggaaacgatgtcaactattcacactgggacgatacacaccttcccgggcctgcttgtgtacctggatcttcgacactgggtcacattgagaatgttctgcgtgcacgtgctgagcaactcaaagtgcctattcttactggcaagaatgtgactggcatcgaagacacagacagcggcgtcaaggtgttctgcggcgaggaaacattcgaggctcaatatcttgtcggatgcgatggtggtcggagcaccattcgcaagaaagctgggattccattcatcggcacagagcccgagttcactggctatgctgtccattgcaagctcgacaacccctttttgctcaagcccggcttcaatcgatgcccaaatggcggactctacatcatggccggcccacagcacatctatgctgtagatcacgacatcagctttgaccgagaccaagaagtaacagcggaacactttcaaaaggttctgagaaagctttccggtaccaacgtggttgttgagcaggtcgttctcgcatccgctttcaccgatcgctgcagacaagctgagcagtaccgcaaaggccgagtcttccttgctggcgatgctgcccacatctaccctccattcagcgcccaaggactcaactgcggttacatcgatgcagtgaatctcggttggaagctcgctgctttggtcaagggctatgctactccgggcttgatcgatacataccagcaagagcaacacccaatggctgcgcgagtacttgattgggtgcgagcacaaattgttacccttcgacctgacgctcatggccgggctgtgggcaatgttatgcgcgagttcctctacaccgaagctggtgtgacatactgcatcggtcgacaatggggtcttgatgtgcggtatgagatcggagagggacataagcttgtcggtcgcagcgctctggattacgagctggacgatggctctcgactgggaactaagctcggcgccgcgaacttcacactcgtggcatttggtgactgcagttcagagctcaccagctccgtcaacgagcttaacccagtgctcggattctgcagagcgaagggcgaggagaagtcaggcctccagggcgtccttgtacgtcctgacgggatcgtgtcgtgggcatctgcagatgccctcaacatcgagacgatcaaggccagcctcgaacgctgggtcgcgctgccagctgctgttgagaaatttgaatctgtgtcttacgaggccaaccagttgtccaacattcagacgccattgacgactactgttcgatcgaatatcggacgtgtatttggcaaggatggagagaagtacactggccttccgacagtggtgacggaagtcccggcaacggctgatgcagtatcgatggattacaaggatgacgacgataagatctgagctcttaattaacaattcttcgccagaggtttggtcaagtctccaatcaaggttgtcggct

SEQ ID NO: 67 - DNA, 1716 bp, Artificial Sequence; identical to SEQ ID NO: 10
  Description of Artificial Sequence Synthetic polynucleotide

SEQ ID NO: 68 - DNA, 1797 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgggcgagaccgatgttctgatagttggtggagggcccactggattgatgctcgccctcgagcttaccagccaagacatcccattccgcatcatcgactcaagtcctattcgaagcgacaaaagccgggccttggttctccattcacggactctggaacttctcaatcgccatggaattgctcaggagttcgttgaccgtggaaatttcaatttggccgtgcgcatcttcgccaaccagaagttcgtgtttgagaacgatttcaacatcatcgctttcaatgacactatgtaccagaaccccttgattatcagtcaggcagagattgaatcgatcctggacgaagtcttgatgaaacatcaagtgaaggtggaacggcccgtgacagcggagaagatatcacaagatgagactggagttactgccgtactacgccacgaggatggaagcgaagagtcactccgttgcaaatatgctgtaggctgtgacggccctcgcagcattgtacgaaattcggcaggtctacaattcgaaggagcggcgtacccgcaggatttcatcctcgcagatgtgcacatgaaatgggagactagagaatgccttcatatctatcttggcgcctctgggttcatgattgtctttccgatgaaggacaatgtctggcgtctgatctgttctcgcagggaagcactgggcgctgacgctgagccaactcttgagaatttccaggagatgttggacaagctgcttcctgaacctgttcaaatatttgatccagtttggatcagtcgatttaaattacaccatcgaattgcagacaattaccgcgctggtcgaatgctcgtcgcgggcgatgcagcccatgtccattctcctgctggtgggcaagggatgaacaccggtatgcacgatgcagtcaatcttggctggaagcttgcaagtgtcattcggggcgagaacgatgactctctgttggactcgtacaatatcgaacggcgtagagttggccagactctcctgcaaggcacagatagacttttcgagtttatggcaacaacaaatcctttgtatctcttcataaggaactacgtcatgcctttcattatgccctgggccatggcgatacctggccgtcgagctcttgcatatagatttgtatccgagttggggattcgatatcgcaaaagtcccatcgttggacaggcaacgacttggaaaggaacactgaaaggtggagatcgagtacccgacgggagactcctgaaggggaatattgagatcactgttcactcgttgcttggagcacgcaagcacagtctacttctgttctcgggcgtggacggggtcaccagcacagacgatctggagaaagctcatgtggagttcatcgaggccagtggtcggtcgatcccagtatacacaatcacgaaatcatcctcggagaacatagagatcatggatccggaggggctagtgcataggctgtttggctttacagcatccggctttgtgttagttcgacccgatggtcacattgcgttcatcgggcctctcacgtcaatggacgagctcaagacgtggatggagagagtatcgatggattacaaggatgacgacgataagatctgagctcttaattaacaattcttcgccagaggtttggtcaagtctccaatcaaggttgtcggct

SEQ ID NO: 69 - DNA, 1884 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatggattgtgaagtattaattatcggtgctgggcccacaggacttatgcttgccctcgaactttcccgcgaacaaatccccttccgaattctcgatagtcattattctactaagacagcccatcaatctcggtctatagtggttcactcacgttccctggagttacttgcccggcatgacttagccgaaagcttcatcgctaaaggattactcatggaagagattcgcttcttcttgaagcaaacccttaatgggaagagtagttaccctggaagtctcatgaatgatacaatttataaaaaaccacttatcattagacaaccactatcagagcagcttctggaaaaacgtctttgcgagtatggaggcattattgaaaggggggcaagcgctaaaaaaatggtgatagatgaagatggcgagcgggtgtctgttttggtacagcatagaattcaggaagaagaagagaaagtaagagaggaattaatcaattgtaagtatgttgtaggttgtgatggagctaacagtatggtacgaaaatttgcgggtatcgactttccgggtactttataccctcaagagctcattctcattgatgcacaggtagactggaacaataaaatatgtccacatgtttttattgataaggaattcgtgatctttataccgctagacgaaacaggtatcaaccgaatcatctgccggcgtcctccaaatgacaccagcactatctcatccagatcaaatctgttatcaggatcatcgccttaccaagagcctagcctatccgagtttcatgatacagttatttcttccgttcctggtacgactcgtattcacagcccgatttggtcttcctcattccgtgtctcacgtcgacttgctagtacatatagtgtggggcggatcttccttgctggtgacgccgctcatgtccatgcacctattggcggtcaaggtatgaataccggtattcaagatgccgtaaacctgggttggaaacttgcgcgtgttataaaatcaactgctccgtcctctttactcaactcatataatgtagagcgatataaagttggtagtgacactgttgaaaatacagatcgcatgttcaatattttaataactacaaaccctgtaaaaatctggttacgaaactttctttttcgctgggttttgccatatttcctcaaaccagattttgcagacagaaaaattcgttacatatcacaattgtcaattcgatatcgaaatagtcccgttgtaggcacggcttctgtgtggaaaggaattctaagaggcggagatcgggcaccagatggatggatgatgaatcctgaaggagaaaatatcactttgcacaggctttttaggacatcaaatgatcatcttttcttattcagcggcctagacactcttgaatcgcggatggtcaatgatactgtccagcagctaaaactctgcagctcagttcttgtggtacacaaaatatatgacgggaattttaaagataaaacaattatcgatggttacatagacccagggggtaaagttcacaccttgtatcagtttattgagcctggttacgttcttgttcgacccgacggatatatctcatttataggacctatgacttcactagatgaactgaagaattggatgaattcatatatgcagggagtatcgatggattacaaggatgacgacgataagatctgagctcttaattaacaattcttcgccagaggtttggtcaagtctccaatcaaggttgtcggct

SEQ ID NO: 70 - DNA, 1512 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatggaggctaagaaatctttcaaagttatcattgctggtggcagtatcgtcggcctaacgctggccaacgcgctcgagaaggcgggtattgattttcttgtcctcgagaggggcgacattgctccacagctcggcgcatcagtctcaatctactgcaatgcgtccagagtcctcgaccagctgggagtttggaaaggcattcaccagagcacaatcccgctgactgatcggctatacttcgatgaacatggccgcttgtttgaggacaacaagatgatgaggctcatagacgacaagactaagcggcctatcacgttcttgagaagggaggcatatatcagggctctttacgaaaacctgaaggacaagtcgaaggtgaagggtcactctgggctggtgtcctttgccgaagatgatgaaggcgtttcagtattcaccagtaccggcgaggaaatcaggggcagcattcttgtgggagcagacggcatccatagtaccgtccggaagctcatggctgagtctgtcgccaagtcggaccctcagagggctaagaatctcatcgagggcttcacggcaaactaccggacgatcttcggaacgtccaaaaaccagcgaaaagacgacccgagcgtgcagatggtggcagatagtctttcccatacctcctattaccgtggcgtcacgggggtctgcgccgtcggcaacagtggggtgatctactggttcctcttggtgaaggaggacacgccttcgacaatgccgaactgcccgcgctactcggaggcggatgccgaagagacgcttcgtaaattcggccaccttcatatgggaccgggatatactttcaatgacctctgggaactgagggagaaaggagtcatggtaccgttgcaggaaggcatcgtcgaaggaagctggaatagtagcgggagggttgtactcatgggcgacgccgtgcacaagttcactataagcgcggggctgggcgccaatttaggcgtagaaggcgcttgccacctggtgaatgagctcctccctgtcctcaaagagacggaaaaccccggaaagcaagagataaaggatgtgctcgacagatacgaggagaagcatcgtcctcgaactaagatctgtgccgtattgtctaactatttgaccaagtacgaggcgatggagacctggtggctcagacttctgaggttcattattcctcggatcccggacagttacaaggcgaagtcatttgtggacttcatgtatggtgctccgattctcgatttcctgcctcacccgggcaagagctctgcggtatcgatggattacaaggatgacgacgataagatctgagctcttaattaacaattcttcgccagaggtttggtcaagtctccaatcaaggttgtcggct

SEQ ID NO: 71 - DNA, 2145 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgtcggtcttacaattcaatctgaacggcttcgtgcctggcgaccctcatgatctctaccgtgtccaccaagaaaccgccgagatcgcaaacaatgtcccgcaagaagtcgacgtcctcatcattggctctggtccagctggtctggctctagcagcacagctatctctctttccaagcatcaccacaagaatcattgaacaaaaggctgaacgactaatcctaggccaagccgatggcctccaagtccgaagcatcgaaatgttcgaagcatatggcttcagcgaaagagtcctcaaggaagcgtactacatcaacgaaaccaccttctggaagcctgacgagcagcaaccggaccgcatcgtgagaagtggcaggattaaggatactgtagacgatctctccgagttcccgcacgtcatcctcaatcaggcgagagttcatgacttcttccttgatgtcatgctcatgtcgccgacaagattgcaaccgagctattcacgcaaactcaagcagctcagccttcaaccagcacagggcaactcctcgcacgctacgtcctccgtgcaagttcaactcgagagaacagactccagccacgagggcgaagtcgaggatgtacgtgctcgctacgtcgtaggctgtgatggagcccgcagtgccgtccgtcaagccctcggtctcaagctggaaggcgactccgcaaaccaagcttggggtgtcatggacgtacttgctgtaaccgacttcccagactggaggaccaaggttgcaatccactccgcgacacaaggcagcatcttgatcatccccagagaaggaggttaccttgtgcgcctctacattgaactcgacaagctcgacgccaacgaacgtatcgcaaacaagaagattaccgccgacaagctcatcgagaccgcacagcgcatcttccatccttatgctttggatgtcaaagaagtagtctggcactctgtctacgagattggccagcgcctgtgtaccagattcgacaacctcactccggagaccgctacaacagaatccccaaacatctttatcgcaggcgacgcatgccacacccacagccctaaagcaggtcaaggcatgaatgtcagcatgcaagatggctacaatctaggctggaagctcgcctccgtgctcaatggaatctctaacccggccatcctccacacctactccgccgaacgtcaaaaagtcgctcaagacctcatcaactttgaccgagagattgcttcaatgtttagcgccagacccaagagccatgccaaagacacagagggagtcgaccctgctgaattccaaaagtactttgtcaagcagggcctcttcatggctggactgggcacagcttactcgacttctgctatcaccgctggatcagagcatcaacacctggccaaaggtttccccatcggcatgagattgcactcttctccagtcataagattggcagatgcaaagccgatccaactaggtcatgtggtaaaagcagatggaagatggagagtcttcgtcttcgcatcagccgaggatcccgcttcgacctcgtcaagcttccacttggcctgcgaatccctgaccgagcttgtcaacaaggtcaaccctactggtgctgatatcgactccgtcatcgatgtcagaggtattctgcagcagggccatgcttcactgaacatcaaccacatgcctgctatcgtgtggccgcaaaagggaaagtatggactcagagattacgaaaaggtcttctgtgctgaagatgtggatggacatagggatatctttaatgctagggatatcgatcgcagaggctgtatcgtggtcgtacgacctgaccagtacgtcgccaacgttctgccgttgtgcgatcatgatggtttgtcaaggttcttcggaggtttcatgtttgcaagggatgtatcgatggattacaaggatgacgacgataagatctgagctcttaattaacaattcttcgccagaggtttggtcaagtctccaatcaaggttgtcggct

SEQ ID NO: 72 - DNA, 1728 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgaccaagacttacgatgttatcgttgccggcgcaggccccatcggcttgtttctcgcctgtgagctgaagttggcgaacgttgacgtgctagtactagagcgtgacaccaaccccgaatccccctggaaaagcgaaccactcggcatacgaggcttgaatacggtatctgtcgagtccttctatcgacgtgggatgctagacaaggtcacctccatggagcgaccgaacttcttcaagaaaggcccggggttccaattcggaggccacttcgccggcatgctactgaatgccaacaagctcgaaatgtcccgttataagtaccgcctccaaggcccagcattggttcccaacggatcaagtatggaacgtgttgagaaggccctaacagagcgcgctgagagcattggagtagatattctcagggggaagggagtcacggaagtctcgcaggatgacaacggcgttacggtggaagcaggaggcgagactttccatgcgaaatggcttgtcggatgtgatggcgggcggagcacagttcgcaaggccgctggcttcgacttcgtcggaacaaatcctgaatgtaccggttacgcttttttggccgatttagaccatcccgaaaagctgaagccggggttccaaccctctgtaggcggcatgtacatcataggacattggggtaacatgtacttgctagacttcgatggtggcgctttcgatcgtacgcaggagatcacacccgagcaccttaacgaagtcatggcccgtgtgacaggcaccgacgtcaaggtcaccaagattcatcaggcatcctcgtttaccgaccgatctaaacaagcgtcgacttatcgaaaaggccgcgttctcttagccggcgacgcggcgcacatccattcgcctctgggagcacaaggattgaatctagggttgggagatgccgtgaaccttggctggaaattggcagcgacagttcaggcttctaaatctggagaggaaccaaaggacctcagcctactcgattcctacgagaaagagcgatatccgatcgggtcttgggttcttgaatggacacgcgctcagatctcgacgctaaagcccgacatgttcggtatcgctcttcgtaggctcatgagcgatttattggagacgactgatggaacgaatttattcatcgatcgcatctggggtctgtcgctgcggtacgatcttggcgacgaacaccccgccgtcggctgtagcgctcccgatttcgaattccacgacggaacgaggctaggtcccaagctagcgacggggcgaggtctattgctcgatttcggatctaatgcagaactcaaggatcttgtcgggaaatacgagagcaaggttgattatcttagcacagaggcaaaggaccagctgggcctgaaatccttgttgatccgccccgatggcgttgtggcttgggtggctgaagagaagcctgatatggactcggcgaaggccgctctggagagatggtttgggctgggttcgaaagtatcgatggattacaaggatgacgacgataagatctgagctcttaattaacaattcttcgccagaggtttggtcaagtctccaatcaaggttgtcggct

SEQ ID NO: 73 - DNA, 1712 bp, Artificial Sequence
  Description of Artificial Sequence Synthetic polynucleotide
caagtttcagtttcatttttcttgttctattacaactttttttacttcttgctcattagaaagaaagcatagcaatctaatctaagttttaattacaagcggccgcatggaaaaccagctctacgacgtcatcattgttggccgcggtcctgttgggctatttttagcatgcgaacttcgcctcacagggctctctgtcttggttgttgagcgccgctcgagctcagagggagtggcggagactcgcgcttttgttgtccattgccgcacactcgagatcctcgcattccgtggtcttcttggcaagttcctccaggagggcacgaagtctccttggtggcactatggcgtcttggacacccgtttggattttagcagatttggccacgaaacgaagcagaattttgtcctgttaagcccacagttcagaactgaggagatttttctggagcgtgcgaaggaattaggcgtcgacattgttctgggaattcgcgtcgagtccgtggagccttcgactacgtctgttacgatatatggaacatcaagcagtccagatggcggcgctagaagttcgttcaaggcgagaggaaagtatcttgtcggtgcggatggagtgcgcagtaccatccggaaggctacgggctttgaattcccaggtacgccggcaacacacacgggtttgaccggcgaggcaacattgggagtgcccatgccctatccctacattgccaaaaacgaagcagggcttgtgatcgccgtacagctgaatattcccagcggccgggctcgcctcaatgttttcacccctgctcgagctaatgtacccgattcgaccccggtaacgctggaagatttcaacgaagcattacaagaagtcactggtgttgactacaagctctccaacccttgcatgcttggacgcttcaacaacgaagcccgctgcgtcgatacttaccggaagggacgcatctttctcgccggtgatgcaggccaccagcaccttccagctggcggccaagggctcaacgtcggtattcaggaagcaactaaccttggatggaaacttggggccgtgattcgcggctatgctccggactcgctattggatacttatgagaccgaaagacttccaatcgcacaaggcgtcgtgcagagcaccactgctcagtctgttctcttttttgcgcacagcgggccggagttggcaatacgaagtgtggtgaacacgttgctcaaaataccaacagcaaaccacgacattgctgtaggagttagtggtttcggcgtctcatacccaaagcttcttgacatgatacttccagctggatgggaggcacttccagaggcgatcgccgggaagcgagccctagatgtgaagttacgcgtcgggaatggcgaggaaaagcagctaagcgactatatgaggggagggaagtgggttcaacttcggttccttgacaaactcgcaggaaggccgccactgccggcgttcgagggggcgacagaggttgtggatgtcgctgaagttctggaggggaaagggagcatgtaccggggcgccttgagcgaactgctaattcgcccggacggttatctagggtttggaaagcgcgtatcgatggattacaaggatgacgacgataagatctgagctcttaattaacaattcttcgccagaggtttggtcaagtctccaatcaaggttgtcggct

SEQ ID NO: 74 - DNA, 1710 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgacctacactactcatgacgtggtgattgtgggagctggtccagttggactcttcctcgcttgcgaactacgccttgccggtctctccgttctggtggtcgagaaacgaacaaattcggacggcatggcggaaacgcgtgcctttgtgatgcatggtcgatctttggaaatcttcgcctctcgcggtctgctcgactctttcgttgaagcgggtcaaaagaccgactggtggcattacggtgttctcgacactcgtcttgattacagtgttttcggccgtgaaacggaccagaactacgtgttactcgtgcctcagtacaaaaccgagatgatcttatttcaacgcgccgtggatctgggtgcagtgattatcaagggtgtccaggtcgattcaatacacgaatccgggccttatgtcgttgcgaggggtttctactgcaataacaaacctttcattgccagcgggaaatacctcgtcggtgcggacggtgttcgcagcacgatccgcaaaatcgccaacatcgagttcactggcaatccacctgtcaatacagtcatgagtggcgaggctacactaggcacggccatgccgaatccctacattgttcacaacgagcacggcctagttatcgccgctgacctgagggtccccagcggaagaacccgcctaaatgtgttcgcgagtgatcgcggcacggttcctgagtcagttgaagtgactcttgaggagatgaatcagagcttgcagaaaataaccggcgttgactacaagctttctaacccttgcatgctaaagcgtttcagcaacgaacaacggctagcgacaacgtatcgccaaaatcggattttcatcgtcggagatgcatgccataaacacctcccagccggcggccagggcttaaatgtcggtctccaagaagctctgaatttaggatggaaacttgccgcagtcatctccaagtccgcccctgcttcgttactcgatacgtacgaagaacgatggccagttgccaaagctgttgtacaaaacacaacttcacagtcactcttattttttgcctcgtctggcccagaatgggcggttagggaagcgatcgacaagcttctgcgtgtaccagaggccaacaagcgtttggctagggagatcagcggattttctgttgcctaccccaaatcgctggatatgatacttccagacggatggcgggccctaccagagaacatccagggaaagcgtgcattgaatgtaaagatgagattaccagatgggatggtaaccgaacttcgtgattacacccaagacggacgatggatccagctgcaccttcctggaaagcatatcattgaactccggcctcctccggcatttggtaactggacgacggtagtggaagttgtcgacatgccagatgaagaagagaagacgagcttgtatatgtgcggagtgagagagatgctgatccgcccggatggctatttggcctttggtcgaatggacgacgtatcgatggattacaaggatgacgacgataagatctgagctcttaattaacaattcttcgccagaggtttggtcaagtctccaatcaaggttgtcggct

SEQ ID NO: 75 - DNA, 1728 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgactaatcaatatgatgttgtaattattggagctgggccagttggattattattggcttgtgaattagctttgggaaaaacaagcgtcatagttctggaaagagaagcgtcgcccgtatctccgtggaaagaagaaccgactggtatgagaggtttgcacctgccttctagtgaaattttgtggcgtaggggtttgttagaaaaaatctggacattggagggtagaccacatggcccgactaagactcctggatttcaatttgcaggccatttcgccgggatacctcttaacctatctcagctagacttagataggtggaaatatcgattgaggggtccatcattaatggggggtccaataacaattgcattaattgagagagtgttagcagaacgcgctgaatcattgggagtcgaaattcatcggggttgcgggttcgagaggatagtccaacaatccgctgacggcgtaacaatagaagcgggtgaagaaacccagacatttcatgcaaaatggcttattggttgcgatggcggtcgttcccaagtccgtaaggctgccgaaatcgactttcctggcactgacgcgaccttaactggctacgtgatccattgtgatcttgaccatcctgaacgattgtcacctggtttccaacctaccaaacagggaatgtatatttttagaaagcccggcgctctctacctgatggatttcgacggtggtgccggccaaacaagagaaccaagccagcagagactacaagaagtgatggagagggttgtagggagaactgatatcaagctagagaaggttcacctagcaagtgcgtttacggacaggggcaaacaagccacaacgtataggaagggaagagtcctcttggctggtgacgcagcgcacatccacccaccacttggtgcacaaggtatgaacgccggtttgggtgatgccatgaatctaggatggaagttagctgcaaccgttcgtggagaagagaaaggagtccgggctttcaccgtgttggatacctatacttcagagagacatcccgttggtcaatgggtcttagaatggaattacgcacaagttgctgctctaaaacccgatgtaacaggttacgccgtacaaaaattaatgagggatttgattgcaactgatgacggtacgaattacttcatagataatgtgtggggattgtctcagcgctatggtgacggtgaaggtgttcatccagtagttggaagatcggcccctgatttcacatttaaagatggtagtagacttgggccaaaattagagggtggcagaggactttttatagattttgaagatgtggatctggagaaagcagttaagcgattcgaaagagttgattatctcggacaaaacgtggaagatagacgtggaatcagagctttacttattagaccagatggatttgttgcttgggctgtagaagagggagatgaaccagacgcagagggtttagaaactgaattaaaaaagtggttttcttacgtatcgatggattacaaggatgacgacgataagatctgagctcttaattaacaattcttcgccagaggtttggtcaagtctccaatcaaggttgtcggct

SEQ ID NO: 76 - DNA, 1725 bp, Artificial Sequence
  Description of Artificial Sequence Synthetic polynucleotide
tcaagtttcagtttcatttttcttgttctattacaactttttttacttcttgctcattagaaagaaagcatagcaatctaatctaagttttaattacaagcggccgcatgggcaagacctgggacgttattattgctggcgctgggccggtcggtcttttcctcgcctgtgaactggccatagctggcgtgtcggtacttgtactggagcgagagatgcagccagagtcgccttggaaagagggattgtttgggcgtcgcggtctctataccccggcggtcgaggcattttaccgccgtggcatcttgaagagaattttcggtgatgatgagcgtccgacgcatttgaagaagactgaaggctttcaatatggaggacactttgcgggcttggtgcttaatgcaaacaacatcgagttttcacgctggccatatcgcttgccaggaccgtcctttcttcctggtccgacttctcttggacgtcttgaggctgtgttgtcggaacgtgcagagacgctgggtgtccagatccttagaggtatggaagtctcccggctcgctgatgaaggtgaatcagtcaaggtatgggcgggcgaccagtggtttacggctcaatggcttgtcggatgtgacggtggacgcagcacagttcggaaaactgctggattcgagtttgtcgggaccgaagcagaattcactggttatattgcggtatgtgatctcgaccgaccagacttattgaagtcaggtttcaagcacacaaactcggggatgtacatagttagcggacctggacagttgcatgttatcgattttgataggagctttgatcggtcacaaactatcacgcgggaacacttccaggaagtgctacggcgagtgtcgggcacaaacatcgttgtcgaagcgcttcacctagcctcctccttcaccgatcgctcgaagcaagcaacagaatatcgtagaggacgcatccttctggctggtgacagtgcccacacacactctccactgggcgcgcaaggattgagtacaggaattggcgatgcgatgaatctcggctggaagcttgcggctaccgtcaaggggtttgcatctcctggtctgctagacacctaccaccaagagcggcatccagaggcagctcgagtgcttgagtggactcgcgctcaggtggcggccttacgccctgatccatatggccaggctattgcgagtctcatgcgggacatgatcaatacccaagatggggcgacttatctcgccgatcgcatctggggactttcagtgcgctatggcccgggcgacgcgcatccacttgtcggttctagcgctccggattttgaattcgatgatggaatgcgacttggagctaagttagagacaggatccttcttggtgattgactttgggagcaacaaccaggtggcagagcatgtgcagtccttgcagtctctgcagttcatgattcaatactgtgcatgtagcgcaaaggaggaattcgggctcaaggggttactcttgcgacctgatggtgtggtcgcatgggtgtcgacggaggagataaatataatacgactgcatgttgcattgtctcgttggataagtctccctagtttcgaggctgtatcgatggattacaaggatgacgacgataagatctgagctcttaattaacaattcttcgccagaggtttggtcaagtctccaatcaaggttgtcggct

SEQ ID NO: 77 - DNA, 1698 bp, Artificial Sequence
  Description of Artificial Sequence Synthetic polynucleotide
atgatgggtgcaaaacacgttattagaagagtgaccaattcggaaaatgatacaggatcaagttcgaaatccactacttcatctaattccgacagggaccataatccctcttcctctaatagagacagatatcacaatgaaacatcacctgccaatatgatattgataaacgagccacctcctaccacctccacagaattggagagcgacactcatattaacgtacgtagcgtttctccatgtttggaaactggttccctaaaagcagagcaagcgcctcagaatgtccagaacccaccacagacacaggaccaagctactgaagatactgccaccaactctcaaccggcctcaacgcagttcagagctatcattgtgggaggtggcccaaacggtttgtgtctggcacatgcgttatacctagctggtattgattatgttttgctagagcgcggtggtgaaatcatcaaccaatctggagccggacttgctttatggcctcattcagttcgtatattagatcaattgggtctgctagacgaagctaggaaacattatttcccgattaagacaaagcataaccaccgccccgacgggtcagtcagagatgttaacgatatgtttgccaaggtagaagttaatcacggtcacccctggatgctgttccatcgagtaacattgctagaactcttgtgggaaaatttgcctgataaagagactagagtaaaagtcaacaagaaggtggaaagtgtagtctccaaccatgatggcgtcgtggttacatgttcagatggaacatctgaagcaggaagtatagtgataggttgcgatggtgtacactcgactgttagacaagtcatgcatgacttgagaagcgagaaaaagaagaaaaggccaggccgaaagctctctctgggaaggtcgccgtcacaagacaagccaatgagagcccactactatgggcttattggttggattccgcttcttgatgggttacaacctgctgcttgctacgaagtgagaagtgagcctaaaggtaaaacctttaccattttaacgggcgaagataccgcttacttcatcgtgtatattcatttagaaaaacccacgagagagcgcagcaggcacacagacgaggatgccgaaaggttggcagccgcattggccgagaacaagataacgagggaaattacttttggtgacctatggagatcaaggcgttggggcaaaatgcttgattttcaagaagggttcgtagataaatggtatcatgaaagaatcgttttagttggcgatgctgtccataagatgactccaaatgcgggactaggcttgaatgctggctggcaaggtatcgctgaattaacgaacagattgcggagattagttgttgcagaaggacaaagaccagatgcaaggtgcgttgaaaaagtttttcgaggttaccaagatagtagaaagggtatggcaaagaaaactatgaggttttcttctctgtacacccgtgtggtagctaatcagagcttactgtatcgtttttgtgatagaatgacaccagcggttggtggggatgtcgcattattaaatacaatggctagtcctattgttaaaaaaggtgtcactttcgactttgtagcggaacgtgatcataaagaaggtagagtaagatgggttcatccacaacatgttccagctgaa

SEQ ID NO: 78 - DNA, 1572 bp, Artificial Sequence
  Description of Artificial Sequence Synthetic polynucleotide
atgaccaagtccaagttatacgacgtgataatcgcgggcgccggacccgtcggcctcttcctcgcccacgaactcagtctccgccaaatagcagtcctagtcctagaacgaacaacccaacaagactcgccctggaaatccggacctctaggcctccgaggcctaaacatccaatccatcgaagcattctatcgccgcggtttactaggccaattattcaatctggacgaaaggcaaaagtaccaaccgagcaagaaaccaggtttccaattcggcggccacttcgccggcatcataataaatgccaatcaattcgatttgcaacgctggaagtaccgcctctcaggtccggcgcttggtccgggtccaacgacgatggatcaagtggagaaggtcctaacggcgcgggtggagagtctaggcgtgaagattctccgcggtcgatctgttagccgtatctcgcaggatgaggcaggcgttacagtcgaaacagacgatgaccattccgagtcatttcggggcaaatggctcgttggctgtgacggtgggcgcagcgttgttcgaaaagaagccggcttcgaattcgtcggtacagatgcccaattcaccggctacgcaacgcatgtcgagatagaaggtcgcgagaagttgaagcctggtctcaacgtttctgaaacggggatgtatatcaacgtagtgcagaatggcgcttttcatctccttgaatttgacggggcgaacttcgatcgcgcgggtgatatcacgatcgatcaccttcagcgggtccttgatcgggctattggtcggacagatgtgaagattacaaatgtgcatcttgcttcttgttataccgatcgttcaatgcaggcggcaagttaccggaggaaccgcgttcttcttgccggtgatgcagcgcatattcattctccgctcggcgggcaaggactgaatgtcggactaggtgatgcgatgaatttggggtggaagcttgcggctatagttcagcgggaccaaggccagggagaggattctgggaatacgcatgatctgactcttctcgatacctacgagagcgagagacaccccattgctgcgtcggttcttgattggactcgtgcgcaggtcacggcgctgaggccggatgcctttggtcgggctacgcgcgccctgacggaagatctgatcaagacggatgatggggctaatctgtttattgatcggatctggggactttcgcagcgatataaacttggcgaagagtcgaggcctacacatccgcttcttgggtctagtgcgcctgatttcgagtttgggaatgggtctaggcttggctcgaagcttgaggagggacgggggttacttcttgattttgggaacagtagtgaactggaatcagttgttgatagaaaatattctgggaaggtagattatcttgctccagaggcaaaggagaactgtgggctgagtgccttgttgattcgacctgatggaatcgttgcatgggtggtggaagaagatgcgcagcacgatattgatgctttgaaagctgcattaggggattggttcactttg

SEQ ID NO: 79 - DNA, 1683 bp, Artificial Sequence
  Description of Artificial Sequence Synthetic polynucleotide
atgacaactaatccagaagtcatcatcgtgggcggcggagcagtcggcctggccgctgcatgcgaattggccatcaagaatgtctctgttacgatcttggagcgggaatctggacctgcagcgccatggaagaacgagaagctgggctttcgcgagctgtttgtgcctgccatcgagcacttgtaccgccgtggcctgctgaacgacatgttccgcaatgaagaacgaccacagactatgccggagaagaagggtggctttgagtttgctggtcattttgctggtatgatgatcaatggaaacgatgtcaactattcacactgggacgatacacaccttcccgggcctgcttgtgtacctggatcttcgacactgggtcacattgagaatgttctgcgtgcacgtgctgagcaactcaaagtgcctattcttactggcaagaatgtgactggcatcgaagacacagacagcggcgtcaaggtgttctgcggcgaggaaacattcgaggctcaatatcttgtcggatgcgatggtggtcggagcaccattcgcaagaaagctgggattccattcatcggcacagagcccgagttcactggctatgctgtccattgcaagctcgacaacccctttttgctcaagcccggcttcaatcgatgcccaaatggcggactctacatcatggccggcccacagcacatctatgctgtagatcacgacatcagctttgaccgagaccaagaagtaacagcggaacactttcaaaaggttctgagaaagctttccggtaccaacgtggttgttgagcaggtcgttctcgcatccgctttcaccgatcgctgcagacaagctgagcagtaccgcaaaggccgagtcttccttgctggcgatgctgcccacatctaccctccattcagcgcccaaggactcaactgcggttacatcgatgcagtgaatctcggttggaagctcgctgctttggtcaagggctatgctactccgggcttgatcgatacataccagcaagagcaacacccaatggctgcgcgagtacttgattgggtgcgagcacaaattgttacccttcgacctgacgctcatggccgggctgtgggcaatgttatgcgcgagttcctctacaccgaagctggtgtgacatactgcatcggtcgacaatggggtcttgatgtgcggtatgagatcggagagggacataagcttgtcggtcgcagcgctctggattacgagctggacgatggctctcgactgggaactaagctcggcgccgcgaacttcacactcgtggcatttggtgactgcagttcagagctcaccagctccgtcaacgagcttaacccagtgctcggattctgcagagcgaagggcgaggagaagtcaggcctccagggcgtccttgtacgtcctgacgggatcgtgtcgtgggcatctgcagatgccctcaacatcgagacgatcaaggccagcctcgaacgctgggtcgcgctgccagctgctgttgagaaatttgaatctgtgtcttacgaggccaaccagttgtccaacattcagacgccattgacgactactgttcgatcgaatatcggacgtgtatttggcaaggatggagagaagtacactggccttccgacagtggtgacggaagtcccggcaacggctgatgca

SEQ ID NO: 80 - DNA, 1590 bp, Artificial Sequence
  Description of Artificial Sequence Synthetic polynucleotide
atgggcgagaccgatgttctgatagttggtggagggcccactggattgatgctcgccctcgagcttaccagccaagacatcccattccgcatcatcgactcaagtcctattcgaagcgacaaaagccgggccttggttctccattcacggactctggaacttctcaatcgccatggaattgctcaggagttcgttgaccgtggaaatttcaatttggccgtgcgcatcttcgccaaccagaagttcgtgtttgagaacgatttcaacatcatcgctttcaatgacactatgtaccagaaccccttgattatcagtcaggcagagattgaatcgatcctggacgaagtcttgatgaaacatcaagtgaaggtggaacggcccgtgacagcggagaagatatcacaagatgagactggagttactgccgtactacgccacgaggatggaagcgaagagtcactccgttgcaaatatgctgtaggctgtgacggccctcgcagcattgtacgaaattcggcaggtctacaattcgaaggagcggcgtacccgcaggatttcatcctcgcagatgtgcacatgaaatgggagactagagaatgccttcatatctatcttggcgcctctgggttcatgattgtctttccgatgaaggacaatgtctggcgtctgatctgttctcgcagggaagcactgggcgctgacgctgagccaactcttgagaatttccaggagatgttggacaagctgcttcctgaacctgttcaaatatttgatccagtttggatcagtcgatttaaattacaccatcgaattgcagacaattaccgcgctggtcgaatgctcgtcgcgggcgatgcagcccatgtccattctcctgctggtgggcaagggatgaacaccggtatgcacgatgcagtcaatcttggctggaagcttgcaagtgtcattcggggcgagaacgatgactctctgttggactcgtacaatatcgaacggcgtagagttggccagactctcctgcaaggcacagatagacttttcgagtttatggcaacaacaaatcctttgtatctcttcataaggaactacgtcatgcctttcattatgccctgggccatggcgatacctggccgtcgagctcttgcatatagatttgtatccgagttggggattcgatatcgcaaaagtcccatcgttggacaggcaacgacttggaaaggaacactgaaaggtggagatcgagtacccgacgggagactcctgaaggggaatattgagatcactgttcactcgttgcttggagcacgcaagcacagtctacttctgttctcgggcgtggacggggtcaccagcacagacgatctggagaaagctcatgtggagttcatcgaggccagtggtcggtcgatcccagtatacacaatcacgaaatcatcctcggagaacatagagatcatggatccggaggggctagtgcataggctgtttggctttacagcatccggctttgtgttagttcgacccgatggtcacattgcgttcatcgggcctctcacgtcaatggacgagctcaagacgtggatggagaga

SEQ ID NO: 81 - DNA, 1677 bp, Artificial Sequence
  Description of Artificial Sequence Synthetic polynucleotide
atggattgtgaagtattaattatcggtgctgggcccacaggacttatgcttgccctcgaactttcccgcgaacaaatccccttccgaattctcgatagtcattattctactaagacagcccatcaatctcggtctatagtggttcactcacgttccctggagttacttgcccggcatgacttagccgaaagcttcatcgctaaaggattactcatggaagagattcgcttcttcttgaagcaaacccttaatgggaagagtagttaccctggaagtctcatgaatgatacaatttataaaaaaccacttatcattagacaaccactatcagagcagcttctggaaaaacgtctttgcgagtatggaggcattattgaaaggggggcaagcgctaaaaaaatggtgatagatgaagatggcgagcgggtgtctgttttggtacagcatagaattcaggaagaagaagagaaagtaagagaggaattaatcaattgtaagtatgttgtaggttgtgatggagctaacagtatggtacgaaaatttgcgggtatcgactttccgggtactttataccctcaagagctcattctcattgatgcacaggtagactggaacaataaaatatgtccacatgtttttattgataaggaattcgtgatctttataccgctagacgaaacaggtatcaaccgaatcatctgccggcgtcctccaaatgacaccagcactatctcatccagatcaaatctgttatcaggatcatcgccttaccaagagcctagcctatccgagtttcatgatacagttatttcttccgttcctggtacgactcgtattcacagcccgatttggtcttcctcattccgtgtctcacgtcgacttgctagtacatatagtgtggggcggatcttccttgctggtgacgccgctcatgtccatgcacctattggcggtcaaggtatgaataccggtattcaagatgccgtaaacctgggttggaaacttgcgcgtgttataaaatcaactgctccgtcctctttactcaactcatataatgtagagcgatataaagttggtagtgacactgttgaaaatacagatcgcatgttcaatattttaataactacaaaccctgtaaaaatctggttacgaaactttctttttcgctgggttttgccatatttcctcaaaccagattttgcagacagaaaaattcgttacatatcacaattgtcaattcgatatcgaaatagtcccgttgtaggcacggcttctgtgtggaaaggaattctaagaggcggagatcgggcaccagatggatggatgatgaatcctgaaggagaaaatatcactttgcacaggctttttaggacatcaaatgatcatcttttcttattcagcggcctagacactcttgaatcgcggatggtcaatgatactgtccagcagctaaaactctgcagctcagttcttgtggtacacaaaatatatgacgggaattttaaagataaaacaattatcgatggttacatagacccagggggtaaagttcacaccttgtatcagtttattgagcctggttacgttcttgttcgacccgacggatatatctcatttataggacctatgacttcactagatgaactgaagaattggatgaattcatatatgcaggga

SEQ ID NO: 82 - DNA, 1305 bp, Artificial Sequence
  Description of Artificial Sequence Synthetic polynucleotide
atggaggctaagaaatctttcaaagttatcattgctggtggcagtatcgtcggcctaacgctggccaacgcgctcgagaaggcgggtattgattttcttgtcctcgagaggggcgacattgctccacagctcggcgcatcagtctcaatctactgcaatgcgtccagagtcctcgaccagctgggagtttggaaaggcattcaccagagcacaatcccgctgactgatcggctatacttcgatgaacatggccgcttgtttgaggacaacaagatgatgaggctcatagacgacaagactaagcggcctatcacgttcttgagaagggaggcatatatcagggctctttacgaaaacctgaaggacaagtcgaaggtgaagggtcactctgggctggtgtcctttgccgaagatgatgaaggcgtttcagtattcaccagtaccggcgaggaaatcaggggcagcattcttgtgggagcagacggcatccatagtaccgtccggaagctcatggctgagtctgtcgccaagtcggaccctcagagggctaagaatctcatcgagggcttcacggcaaactaccggacgatcttcggaacgtccaaaaaccagcgaaaagacgacccgagcgtgcagatggtggcagatagtctttcccatacctcctattaccgtggcgtcacgggggtctgcgccgtcggcaacagtggggtgatctactggttcctcttggtgaaggaggacacgccttcgacaatgccgaactgcccgcgctactcggaggcggatgccgaagagacgcttcgtaaattcggccaccttcatatgggaccgggatatactttcaatgacctctgggaactgagggagaaaggagtcatggtaccgttgcaggaaggcatcgtcgaaggaagctggaatagtagcgggagggttgtactcatgggcgacgccgtgcacaagttcactataagcgcggggctgggcgccaatttaggcgtagaaggcgcttgccacctggtgaatgagctcctccctgtcctcaaagagacggaaaaccccggaaagcaagagataaaggatgtgctcgacagatacgaggagaagcatcgtcctcgaactaagatctgtgccgtattgtctaactatttgaccaagtacgaggcgatggagacctggtggctcagacttctgaggttcattattcctcggatcccggacagttacaaggcgaagtcatttgtggacttcatgtatggtgctccgattctcgatttcctgcctcacccgggcaagagctctgcg

SEQ ID NO: 83 - DNA, 1938 bp, Artificial Sequence
  Description of Artificial Sequence Synthetic polynucleotide
atgtcggtcttacaattcaatctgaacggcttcgtgcctggcgaccctcatgatctctaccgtgtccaccaagaaaccgccgagatcgcaaacaatgtcccgcaagaagtcgacgtcctcatcattggctctggtccagctggtctggctctagcagcacagctatctctctttccaagcatcaccacaagaatcattgaacaaaaggctgaacgactaatcctaggccaagccgatggcctccaagtccgaagcatcgaaatgttcgaagcatatggcttcagcgaaagagtcctcaaggaagcgtactacatcaacgaaaccaccttctggaagcctgacgagcagcaaccggaccgcatcgtgagaagtggcaggattaaggatactgtagacgatctctccgagttcccgcacgtcatcctcaatcaggcgagagttcatgacttcttccttgatgtcatgctcatgtcgccgacaagattgcaaccgagctattcacgcaaactcaagcagctcagccttcaaccagcacagggcaactcctcgcacgctacgtcctccgtgcaagttcaactcgagagaacagactccagccacgagggcgaagtcgaggatgtacgtgctcgctacgtcgtaggctgtgatggagcccgcagtgccgtccgtcaagccctcggtctcaagctggaaggcgactccgcaaaccaagcttggggtgtcatggacgtacttgctgtaaccgacttcccagactggaggaccaaggttgcaatccactccgcgacacaaggcagcatcttgatcatccccagagaaggaggttaccttgtgcgcctctacattgaactcgacaagctcgacgccaacgaacgtatcgcaaacaagaagattaccgccgacaagctcatcgagaccgcacagcgcatcttccatccttatgctttggatgtcaaagaagtagtctggcactctgtctacgagattggccagcgcctgtgtaccagattcgacaacctcactccggagaccgctacaacagaatccccaaacatctttatcgcaggcgacgcatgccacacccacagccctaaagcaggtcaaggcatgaatgtcagcatgcaagatggctacaatctaggctggaagctcgcctccgtgctcaatggaatctctaacccggccatcctccacacctactccgccgaacgtcaaaaagtcgctcaagacctcatcaactttgaccgagagattgcttcaatgtttagcgccagacccaagagccatgccaaagacacagagggagtcgaccctgctgaattccaaaagtactttgtcaagcagggcctcttcatggctggactgggcacagcttactcgacttctgctatcaccgctggatcagagcatcaacacctggccaaaggtttccccatcggcatgagattgcactcttctccagtcataagattggcagatgcaaagccgatccaactaggtcatgtggtaaaagcagatggaagatggagagtcttcgtcttcgcatcagccgaggatcccgcttcgacctcgtcaagcttccacttggcctgcgaatccctgaccgagcttgtcaacaaggtcaaccctactggtgctgatatcgactccgtcatcgatgtcagaggtattctgcagcagggccatgcttcactgaacatcaaccacatgcctgctatcgtgtggccgcaaaagggaaagtatggactcagagattacgaaaaggtcttctgtgctgaagatgtggatggacatagggatatctttaatgctagggatatcgatcgcagaggctgtatcgtggtcgtacgacctgaccagtacgtcgccaacgttctgccgttgtgcgatcatgatggtttgtcaaggttcttcggaggtttcatgtttgcaagggat

SEQ ID NO: 84 - DNA, 1521 bp, Artificial Sequence
  Description of Artificial Sequence Synthetic polynucleotide
atgaccaagacttacgatgttatcgttgccggcgcaggccccatcggcttgtttctcgcctgtgagctgaagttggcgaacgttgacgtgctagtactagagcgtgacaccaaccccgaatccccctggaaaagcgaaccactcggcatacgaggcttgaatacggtatctgtcgagtccttctatcgacgtgggatgctagacaaggtcacctccatggagcgaccgaacttcttcaagaaaggcccggggttccaattcggaggccacttcgccggcatgctactgaatgccaacaagctcgaaatgtcccgttataagtaccgcctccaaggcccagcattggttcccaacggatcaagtatggaacgtgttgagaaggccctaacagagcgcgctgagagcattggagtagatattctcagggggaagggagtcacggaagtctcgcaggatgacaacggcgttacggtggaagcaggaggcgagactttccatgcgaaatggcttgtcggatgtgatggcgggcggagcacagttcgcaaggccgctggcttcgacttcgtcggaacaaatcctgaatgtaccggttacgcttttttggccgatttagaccatcccgaaaagctgaagccggggttccaaccctctgtaggcggcatgtacatcataggacattggggtaacatgtacttgctagacttcgatggtggcgctttcgatcgtacgcaggagatcacacccgagcaccttaacgaagtcatggcccgtgtgacaggcaccgacgtcaaggtcaccaagattcatcaggcatcctcgtttaccgaccgatctaaacaagcgtcgacttatcgaaaaggccgcgttctcttagccggcgacgcggcgcacatccattcgcctctgggagcacaaggattgaatctagggttgggagatgccgtgaaccttggctggaaattggcagcgacagttcaggcttctaaatctggagaggaaccaaaggacctcagcctactcgattcctacgagaaagagcgatatccgatcgggtcttgggttcttgaatggacacgcgctcagatctcgacgctaaagcccgacatgttcggtatcgctcttcgtaggctcatgagcgatttattggagacgactgatggaacgaatttattcatcgatcgcatctggggtctgtcgctgcggtacgatcttggcgacgaacaccccgccgtcggctgtagcgctcccgatttcgaattccacgacggaacgaggctaggtcccaagctagcgacggggcgaggtctattgctcgatttcggatctaatgcagaactcaaggatcttgtcgggaaatacgagagcaaggttgattatcttagcacagaggcaaaggaccagctgggcctgaaatccttgttgatccgccccgatggcgttgtggcttgggtggctgaagagaagcctgatatggactcggcgaaggccgctctggagagatggtttgggctgggttcgaaa

SEQ ID NO: 85 - DNA, 1506 bp, Artificial Sequence
  Description of Artificial Sequence Synthetic polynucleotide
atggaaaaccagctctacgacgtcatcattgttggccgcggtcctgttgggctatttttagcatgcgaacttcgcctcacagggctctctgtcttggttgttgagcgccgctcgagctcagagggagtggcggagactcgcgcttttgttgtccattgccgcacactcgagatcctcgcattccgtggtcttcttggcaagttcctccaggagggcacgaagtctccttggtggcactatggcgtcttggacacccgtttggattttagcagatttggccacgaaacgaagcagaattttgtcctgttaagcccacagttcagaactgaggagatttttctggagcgtgcgaaggaattaggcgtcgacattgttctgggaattcgcgtcgagtccgtggagccttcgactacgtctgttacgatatatggaacatcaagcagtccagatggcggcgctagaagttcgttcaaggcgagaggaaagtatcttgtcggtgcggatggagtgcgcagtaccatccggaaggctacgggctttgaattcccaggtacgccggcaacacacacgggtttgaccggcgaggcaacattgggagtgcccatgccctatccctacattgccaaaaacgaagcagggcttgtgatcgccgtacagctgaatattcccagcggccgggctcgcctcaatgttttcacccctgctcgagctaatgtacccgattcgaccccggtaacgctggaagatttcaacgaagcattacaagaagtcactggtgttgactacaagctctccaacccttgcatgcttggacgcttcaacaacgaagcccgctgcgtcgatacttaccggaagggacgcatctttctcgccggtgatgcaggccaccagcaccttccagctggcggccaagggctcaacgtcggtattcaggaagcaactaaccttggatggaaacttggggccgtgattcgcggctatgctccggactcgctattggatacttatgagaccgaaagacttccaatcgcacaaggcgtcgtgcagagcaccactgctcagtctgttctcttttttgcgcacagcgggccggagttggcaatacgaagtgtggtgaacacgttgctcaaaataccaacagcaaaccacgacattgctgtaggagttagtggtttcggcgtctcatacccaaagcttcttgacatgatacttccagctggatgggaggcacttccagaggcgatcgccgggaagcgagccctagatgtgaagttacgcgtcgggaatggcgaggaaaagcagctaagcgactatatgaggggagggaagtgggttcaacttcggttccttgacaaactcgcaggaaggccgccactgccggcgttcgagggggcgacagaggttgtggatgtcgctgaagttctggaggggaaagggagcatgtaccggggcgccttgagcgaactgctaattcgcccggacggttatctagggtttggaaagcgc

SEQ ID NO: 86 - DNA, 1503 bp, Artificial Sequence
  Description of Artificial Sequence Synthetic polynucleotide
atgacctacactactcatgacgtggtgattgtgggagctggtccagttggactcttcctcgcttgcgaactacgccttgccggtctctccgttctggtggtcgagaaacgaacaaattcggacggcatggcggaaacgcgtgcctttgtgatgcatggtcgatctttggaaatcttcgcctctcgcggtctgctcgactctttcgttgaagcgggtcaaaagaccgactggtggcattacggtgttctcgacactcgtcttgattacagtgttttcggccgtgaaacggaccagaactacgtgttactcgtgcctcagtacaaaaccgagatgatcttatttcaacgcgccgtggatctgggtgcagtgattatcaagggtgtccaggtcgattcaatacacgaatccgggccttatgtcgttgcgaggggtttctactgcaataacaaacctttcattgccagcgggaaatacctcgtcggtgcggacggtgttcgcagcacgatccgcaaaatcgccaacatcgagttcactggcaatccacctgtcaatacagtcatgagtggcgaggctacactaggcacggccatgccgaatccctacattgttcacaacgagcacggcctagttatcgccgctgacctgagggtccccagcggaagaacccgcctaaatgtgttcgcgagtgatcgcggcacggttcctgagtcagttgaagtgactcttgaggagatgaatcagagcttgcagaaaataaccggcgttgactacaagctttctaacccttgcatgctaaagcgtttcagcaacgaacaacggctagcgacaacgtatcgccaaaatcggattttcatcgtcggagatgcatgccataaacacctcccagccggcggccagggcttaaatgtcggtctccaagaagctctgaatttaggatggaaacttgccgcagtcatctccaagtccgcccctgcttcgttactcgatacgtacgaagaacgatggccagttgccaaagctgttgtacaaaacacaacttcacagtcactcttattttttgcctcgtctggcccagaatgggcggttagggaagcgatcgacaagcttctgcgtgtaccagaggccaacaagcgtttggctagggagatcagcggattttctgttgcctaccccaaatcgctggatatgatacttccagacggatggcgggccctaccagagaacatccagggaaagcgtgcattgaatgtaaagatgagattaccagatgggatggtaaccgaacttcgtgattacacccaagacggacgatggatccagctgcaccttcctggaaagcatatcattgaactccggcctcctccggcatttggtaactggacgacggtagtggaagttgtcgacatgccagatgaagaagagaagacgagcttgtatatgtgcggagtgagagagatgctgatccgcccggatggctatttggcctttggtcgaatggacgac

SEQ ID NO: 87 - DNA, 1521 bp, Artificial Sequence
  Description of Artificial Sequence Synthetic polynucleotide
atgactaatcaatatgatgttgtaattattggagctgggccagttggattattattggcttgtgaattagctttgggaaaaacaagcgtcatagttctggaaagagaagcgtcgcccgtatctccgtggaaagaagaaccgactggtatgagaggtttgcacctgccttctagtgaaattttgtggcgtaggggtttgttagaaaaaatctggacattggagggtagaccacatggcccgactaagactcctggatttcaatttgcaggccatttcgccgggatacctcttaacctatctcagctagacttagataggtggaaatatcgattgaggggtccatcattaatggggggtccaataacaattgcattaattgagagagtgttagcagaacgcgctgaatcattgggagtcgaaattcatcggggttgcgggttcgagaggatagtccaacaatccgctgacggcgtaacaatagaagcgggtgaagaaacccagacatttcatgcaaaatggcttattggttgcgatggcggtcgttcccaagtccgtaaggctgccgaaatcgactttcctggcactgacgcgaccttaactggctacgtgatccattgtgatcttgaccatcctgaacgattgtcacctggtttccaacctaccaaacagggaatgtatatttttagaaagcccggcgctctctacctgatggatttcgacggtggtgccggccaaacaagagaaccaagccagcagagactacaagaagtgatggagagggttgtagggagaactgatatcaagctagagaaggttcacctagcaagtgcgtttacggacaggggcaaacaagccacaacgtataggaagggaagagtcctcttggctggtgacgcagcgcacatccacccaccacttggtgcacaaggtatgaacgccggtttgggtgatgccatgaatctaggatggaagttagctgcaaccgttcgtggagaagagaaaggagtccgggctttcaccgtgttggatacctatacttcagagagacatcccgttggtcaatgggtcttagaatggaattacgcacaagttgctgctctaaaacccgatgtaacaggttacgccgtacaaaaattaatgagggatttgattgcaactgatgacggtacgaattacttcatagataatgtgtggggattgtctcagcgctatggtgacggtgaaggtgttcatccagtagttggaagatcggcccctgatttcacatttaaagatggtagtagacttgggccaaaattagagggtggcagaggactttttatagattttgaagatgtggatctggagaaagcagttaagcgattcgaaagagttgattatctcggacaaaacgtggaagatagacgtggaatcagagctttacttattagaccagatggatttgttgcttgggctgtagaagagggagatgaaccagacgcagagggtttagaaactgaattaaaaaagtggttttcttac

SEQ ID NO: 88 - DNA, 1518 bp, Artificial Sequence
  Description of Artificial Sequence Synthetic polynucleotide
atgggcaagacctgggacgttattattgctggcgctgggccggtcggtcttttcctcgcctgtgaactggccatagctggcgtgtcggtacttgtactggagcgagagatgcagccagagtcgccttggaaagagggattgtttgggcgtcgcggtctctataccccggcggtcgaggcattttaccgccgtggcatcttgaagagaattttcggtgatgatgagcgtccgacgcatttgaagaagactgaaggctttcaatatggaggacactttgcgggcttggtgcttaatgcaaacaacatcgagttttcacgctggccatatcgcttgccaggaccgtcctttcttcctggtccgacttctcttggacgtcttgaggctgtgttgtcggaacgtgcagagacgctgggtgtccagatccttagaggtatggaagtctcccggctcgctgatgaaggtgaatcagtcaaggtatgggcgggcgaccagtggtttacggctcaatggcttgtcggatgtgacggtggacgcagcacagttcggaaaactgctggattcgagtttgtcgggaccgaagcagaattcactggttatattgcggtatgtgatctcgaccgaccagacttattgaagtcaggtttcaagcacacaaactcggggatgtacatagttagcggacctggacagttgcatgttatcgattttgataggagctttgatcggtcacaaactatcacgcgggaacacttccaggaagtgctacggcgagtgtcgggcacaaacatcgttgtcgaagcgcttcacctagcctcctccttcaccgatcgctcgaagcaagcaacagaatatcgtagaggacgcatccttctggctggtgacagtgcccacacacactctccactgggcgcgcaaggattgagtacaggaattggcgatgcgatgaatctcggctggaagcttgcggctaccgtcaaggggtttgcatctcctggtctgctagacacctaccaccaagagcggcatccagaggcagctcgagtgcttgagtggactcgcgctcaggtggcggccttacgccctgatccatatggccaggctattgcgagtctcatgcgggacatgatcaatacccaagatggggcgacttatctcgccgatcgcatctggggactttcagtgcgctatggcccgggcgacgcgcatccacttgtcggttctagcgctccggattttgaattcgatgatggaatgcgacttggagctaagttagagacaggatccttcttggtgattgactttgggagcaacaaccaggtggcagagcatgtgcagtccttgcagtctctgcagttcatgattcaatactgtgcatgtagcgcaaaggaggaattcgggctcaaggggttactcttgcgacctgatggtgtggtcgcatgggtgtcgacggaggagataaatataatacgactgcatgttgcattgtctcgttggataagtctccctagtttcgaggct

SEQ ID NO: 89 - DNA, 45 bp, Artificial Sequence
  Description of Artificial Sequence Synthetic primer
  modified_base (23)..(24): a, c, t, g, unknown or other
agtgcgagcgtggactcctagmnntttcgagaagtcaacaggctc

SEQ ID NO: 90 - DNA, 18 bp, Artificial Sequence
  Description of Artificial Sequence Synthetic primer
ctaggagtccacgctcgc

SEQ ID NO: 91 - DNA, 26 bp, Artificial Sequence
  Description of Artificial Sequence Synthetic primer
caatgcatatgggtgtctagtatcaa

SEQ ID NO: 92 - DNA, 60 bp, Artificial Sequence
  Description of Artificial Sequence Synthetic primer
  modified_base (33)..(34): a, c, t, g, unknown or other
catcatttgatactagacacccatatgcattgnnkgttccacaagtacgaactgaagaac

SEQ ID NO: 93 - DNA, 24 bp, Artificial Sequence
  Description of Artificial Sequence Synthetic primer
atgtgggtcttgacctggaaaatc

SEQ ID NO: 94 - DNA, 60 bp, Artificial Sequence
  Description of Artificial Sequence Synthetic primer
  modified_base (39)..(40): a, c, t, g, unknown or other
aactattgggcatagattttccaggtcaagacccacatnnktttgctgtcatcgcagacg

SEQ ID NO: 95 - DNA, 22 bp, Artificial Sequence
  Description of Artificial Sequence Synthetic primer
tgcacgaaggtcatgtctcatg

SEQ ID NO: 96 - DNA, 45 bp, Artificial Sequence
  Description of Artificial Sequence Synthetic primer
  modified_base (24)..(25): a, c, t, g, unknown or other
tcatgagacatgaccttcgtgcannkttcgcagcatttccgctag

SEQ ID NO: 97 - DNA, 22 bp, Artificial Sequence
  Description of Artificial Sequence Synthetic primer
ccatgcacgaaggtcatgtctc

SEQ ID NO: 98 - DNA, 45 bp, Artificial Sequence
  Description of Artificial Sequence Synthetic primer
  modified_base (23)..(24): a, c, t, g, unknown or other
gagacatgaccttcgtgcatggnnkgcagcatttccgctagaacc

SEQ ID NO: 99 - DNA, 52 bp, Artificial Sequence
  Description of Artificial Sequence Synthetic primer
  modified_base (30)..(31): a, c, t, g, unknown or other
cagcatacggtctatcgaaaaaggcgacmnntgctctgtagacgtctggttc

SEQ ID NO: 100 - DNA, 21 bp, Artificial Sequence
  Description of Artificial Sequence Synthetic primer
gtcgcctttttcgatagaccg

SEQ ID NO: 101 - DNA, 16 bp, Artificial Sequence
  Description of Artificial Sequence Synthetic primer
cggcgccctcctgtca

SEQ ID NO: 102 - DNA, 45 bp, Artificial Sequence
  Description of Artificial Sequence Synthetic primer
  modified_base (23)..(24): a, c, t, g, unknown or other
gtatgctgacaggagggcgccgnnkacggaggaggatgtcagagc

SEQ ID NO: 103 - DNA, 52 bp, Artificial Sequence
  Description of Artificial Sequence Synthetic primer
  modified_base (27)..(28): a, c, t, g, unknown or other
ctaagttcagtccctggccaccagcmnncaaatgaatatggcatgcatcacc

SEQ ID NO: 104 - DNA, 18 bp, Artificial Sequence
  Description of Artificial Sequence Synthetic primer
gctggtggccagggactg

SEQ ID NO: 105 - DNA, 60 bp, Artificial Sequence
  Description of Artificial Sequence Synthetic primer
  modified_base (35)..(36): a, c, t, g, unknown or other
atcttgaaatcctaagttcagtccctggccaccmnngggcaaatgaatatggcatgcatc

SEQ ID NO: 106 - DNA, 22 bp, Artificial Sequence
  Description of Artificial Sequence Synthetic primer
ggtggccagggactgaacttag

SEQ ID NO: 107 - DNA, 45 bp, Artificial Sequence
  Description of Artificial Sequence Synthetic primer
  modified_base (23)..(24): a, c, t, g, unknown or other
tcctaagttcagtccctggccmnnagcgggcaaatgaatatggca

SEQ ID NO: 108 - DNA, 21 bp, Artificial Sequence
  Description of Artificial Sequence Synthetic primer
ggccagggactgaacttagga

SEQ ID NO: 109 - DNA, 60 bp, Artificial Sequence
  Description of Artificial Sequence Synthetic primer
  modified_base (37)..(38): a, c, t, g, unknown or other
ttaacggcatcttgaaatcctaagttcagtccctgmnnaccagcgggcaaatgaatatgg

SEQ ID NO: 110 - DNA, 25 bp, Artificial Sequence
  Description of Artificial Sequence Synthetic primer
cagggactgaacttaggatttcaag

SEQ ID NO: 111 - DNA, 45 bp, Artificial Sequence
  Description of Artificial Sequence Synthetic primer
  modified_base (25)..(26): a, c, t, g, unknown or other
gtgcgagcgtggactcctagagcmnncgagaagtcaacaggctcg

SEQ ID NO: 112 - DNA, 25 bp, Artificial Sequence
  Description of Artificial Sequence Synthetic primer
gctctaggagtccacgctcgcactg

SEQ ID NO: 113 - DNA, 45 bp, Artificial Sequence
  Description of Artificial Sequence Synthetic primer
  modified_base (21)..(22): a, c, t, g, unknown or other
caacagtgcgagcgtggacmnntagagctttcgagaagtcaacag

SEQ ID NO: 114 - DNA, 19 bp, Artificial Sequence
  Description of Artificial Sequence Synthetic primer
gtccacgctcgcactgttg

SEQ ID NO: 115 - DNA, 25 bp, Artificial Sequence
  Description of Artificial Sequence Synthetic primer
tgggtgtctagtatcaaatgatgag

SEQ ID NO: 116 - DNA, 60 bp, Artificial Sequence
  Description of Artificial Sequence Synthetic primer
  modified_base (33)..(34): a, c, t, g, unknown or other
tctcatcatttgatactagacacccatatgcannktttgttccacaagtacgaactgaag

SEQ ID NO: 117 - DNA, 20 bp, Artificial Sequence
  Description of Artificial Sequence Synthetic primer
ggcgactgttgctctgtaga

SEQ ID NO: 118 - DNA, 45 bp, Artificial Sequence
  Description of Artificial Sequence Synthetic primer
  modified_base (21)..(22): a, c, t, g, unknown or other
tctacagagcaacagtcgccnnkttcgatagaccgtatgctgaca

SEQ ID NO: 119 - DNA, 56 bp, Artificial Sequence
  Description of Artificial Sequence Synthetic primer
  modified_base (29)..(30): a, c, t, g, unknown or other
cattaattcccttaaaccctcgtagcgmnnatctggatcaattagaacagcttgag

SEQ ID NO: 120 - DNA, 20 bp, Artificial Sequence
  Description of Artificial Sequence Synthetic primer
cgctacgagggtttaaggga

SEQ ID NO: 121 - DNA, 25 bp, Artificial Sequence
  Description of Artificial Sequence Synthetic primer
aggatctggatcaattagaacagct

SEQ ID NO: 122 - DNA, 60 bp, Artificial Sequence
  Description of Artificial Sequence Synthetic primer
  modified_base (30)..(31): a, c, t, g, unknown or other
ctcaagctgttctaattgatccagatcctnnktacgagggtttaagggaattaatgattg

SEQ ID NO: 123 - DNA, 60 bp, Artificial Sequence
  Description of Artificial Sequence Synthetic primer
  modified_base (32)..(33): a, c, t, g, unknown or other
tagccccgctaaatatctattagtctcaggmnngtgcaacaattcaatcattaattccct

SEQ ID NO: 124 - DNA, 25 bp, Artificial Sequence
  Description of Artificial Sequence Synthetic primer
cctgagactaatagatatttagcgg

SEQ ID NO: 125 - DNA, 23 bp, Artificial Sequence
  Description of Artificial Sequence Synthetic primer
gctcattagaaagaaagcatagc

SEQ ID NO: 126 - DNA, 26 bp, Artificial Sequence
  Description of Artificial Sequence Synthetic primer
ctggcgaagaattgttaattaagagc

SEQ ID NO: 127 - protein, 7 aa, Streptomyces purpurascens
PYPRAAG

SEQ ID NO: 128 - protein, 7 aa, Streptomyces rimosus
FSKALGV

SEQ ID NO: 129 - protein, 6 aa, Streptomyces purpurascens
PRAGQN

SEQ ID NO: 130 - protein, 7 aa, Streptomyces rimosus
SKALGVH

SEQ ID NO: 131 - protein, 7 aa, Streptomyces purpurascens
AAGQNPR

SEQ ID NO: 132 - protein, 8 aa, Streptomyces rimosus
KALGVHAR

SEQ ID NO: 133 - protein, 6 aa, Streptomyces purpurascens
ADDIRG

SEQ ID NO: 134 - protein, 7 aa, Streptomyces purpurascens
QGDFVIR

SEQ ID NO: 135 - protein, 7 aa, Streptomyces purpurascens
QGDFVIR

SEQ ID NO: 136 - protein, 6 aa, Streptomyces purpurascens
DDMVAA

SEQ ID NO: 137 - protein, 6 aa, Streptomyces purpurascens
PAGWAM

SEQ ID NO: 138 - protein, 10 aa, Streptomyces purpurascens
PAGWAMLSQD

SEQ ID NO: 139 - protein, 7 aa, Streptomyces purpurascens
LTHMVGV

SEQ ID NO: 140 - protein, 8 aa, Streptomyces purpurascens
GTTGWYYL

SEQ ID NO: 141 - protein, 7 aa, Streptomyces purpurascens
TGWYYLH

SEQ ID NO: 142 - protein, 13 aa, Streptomyces purpurascens
PEFKGTFGPTDRP

SEQ ID NO: 143 - protein, 8 aa, Streptomyces purpurascens
TFGPTDRP

SEQ ID NO: 144 - protein, 13 aa, Streptomyces purpurascens
RHTLFVEYDPDEG

SEQ ID NO: 145 - protein, 13 aa, Streptomyces purpurascens
VDIQGWEMAARIA

SEQ ID NO: 146 - DNA, 402 bp, Artificial Sequence; identical to SEQ ID NO: 42
  Description of Artificial Sequence Synthetic polynucleotide

SEQ ID NO: 147 - protein, 8 aa, Streptomyces purpurascens
WEMAARIA

SEQ ID NO: 148 - protein, 10 aa, Streptomyces purpurascens
VTPPTGGMSG

SEQ ID NO: 149 - protein, 10 aa, Streptomyces purpurascens
VTPPTGGMSG

SEQ ID NO: 150 - protein, 10 aa, Streptomyces purpurascens
VTPPTGGMSG

SEQ ID NO: 151 - protein, 10 aa, Streptomyces purpurascens
VTPPTGGMSG

SEQ ID NO: 152 - protein, 9 aa, Streptomyces purpurascens
AIYAQRMAP

SEQ ID NO: 153 - protein, 9 aa, Streptomyces purpurascens
AIYAQRMAP

SEQ ID NO: 154 - protein, 7 aa, Streptomyces purpurascens
SVGYPET

SEQ ID NO: 155 - protein, 6 aa, Streptomyces rimosus
PYALFV

SEQ ID NO: 156 - protein, 9 aa, Streptomyces rimosus
PYALFVPQV

SEQ ID NO: 157 - protein, 5 aa, Streptomyces rimosus
HMFAV

SEQ ID NO: 158 - protein, 10 aa, Streptomyces rimosus
DLRAWFAAFP

SEQ ID NO: 159 - protein, 11 aa, Streptomyces rimosus
DLRAWFAAFPL

SEQ ID NO: 160 - protein, 4 aa, Streptomyces rimosus
ATVA

SEQ ID NO: 161 - protein, 8 aa, Streptomyces rimosus
TVAFFDRP

SEQ ID NO: 162 - protein, 9 aa, Streptomyces rimosus
RRAPVTEED

SEQ ID NO: 163 - protein, 10 aa, Streptomyces rimosus
IHLPAGGQGL

SEQ ID NO: 164 - protein, 10 aa, Streptomyces rimosus
IHLPAGGQGL

SEQ ID NO: 165 - protein, 10 aa, Streptomyces rimosus
IHLPAGGQGL

SEQ ID NO: 166 - protein, 10 aa, Streptomyces rimosus
IHLPAGGQGL

SEQ ID NO: 167 - protein, 9 aa, Streptomyces rimosus
LIDPDPRYE

SEQ ID NO: 168 - protein, 9 aa, Streptomyces rimosus
LIDPDPRYE

SEQ ID NO: 169 - protein, 7 aa, Streptomyces rimosus
LLHVPET

SEQ ID NO: 170 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDISTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 171 - protein, 26 aa, Saccharomyces cerevisiae
MTKPTQVLVRSVSILFFITLLHLVVA

SEQ ID NO: 172 - protein, 290 aa, Saccharomyces cerevisiae
LNDVAGPAETAPVSLLPREAPWYDKIWEVKDWLLQRATDGNWGKSITWGSFVASDAGVVIFGINVCKNCVGERKDDISTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 173 - DNA, 951 bp, Saccharomyces cerevisiae
atgaccaaaccgacccaggtgctggtgcgcagcgtgagcattctgttttttattaccctgctgcatctggtggtggcgctgaacgatgtggcgggcccggcggaaaccgcgccggtgagcctgctgccgcgcgaagcgccgtggtatgataaaatttgggaagtgaaagattggctgctgcagcgcgcgaccgatggcaactggggcaaaagcattacctggggcagctttgtggcgagcgatgcgggcgtggtgatttttggcattaacgtgtgcaaaaactgcgtgggcgaacgcaaagatgatattagcaccgattgcggcaaacagaccctggcgctgctggtgagcatttttgtggcggtgaccagcggccatcatctgatttggggcggcaaccgcccggtgagccagagcgatccgaacggcgcgaccgtggcgcgccgcgatattagcaccgtggcggatggcgatattccgctggattttagcgcgctgaacgatattctgaacgaacatggcattagcattctgccggcgaacgcgagccagtatgtgaaacgcagcgataccgcggaacataccaccagctttgtggtgaccaacaactataccagcctgcataccgatctgattcatcatggcaacggcacctataccacctttaccaccccgcatattccggcggtggcgaaacgctatgtgtatccgatgtgcgaacatggcattaaagcgagctattgcatggcgctgaacgatgcgatggtgagcgcgaacggcaacctgtatggcctggcggaaaaactgtttagcgaagatgaaggccagtgggaaaccaactattataaactgtattggagcaccggccagtggattatgagcatgaaatttattgaagaaagcattgataacgcgaacaacgattttgaaggctgcgataccggccattag

SEQ ID NO: 174 - DNA, 78 bp, Saccharomyces cerevisiae
atgaccaaaccgacccaggtgctggtgcgcagcgtgagcattctgttttttattaccctgctgcatctggtggtggcg

SEQ ID NO: 175 - DNA, 873 bp, Saccharomyces cerevisiae
ctgaacgatgtggcgggcccggcggaaaccgcgccggtgagcctgctgccgcgcgaagcgccgtggtatgataaaatttgggaagtgaaagattggctgctgcagcgcgcgaccgatggcaactggggcaaaagcattacctggggcagctttgtggcgagcgatgcgggcgtggtgatttttggcattaacgtgtgcaaaaactgcgtgggcgaacgcaaagatgatattagcaccgattgcggcaaacagaccctggcgctgctggtgagcatttttgtggcggtgaccagcggccatcatctgatttggggcggcaaccgcccggtgagccagagcgatccgaacggcgcgaccgtggcgcgccgcgatattagcaccgtggcggatggcgatattccgctggattttagcgcgctgaacgatattctgaacgaacatggcattagcattctgccggcgaacgcgagccagtatgtgaaacgcagcgataccgcggaacataccaccagctttgtggtgaccaacaactataccagcctgcataccgatctgattcatcatggcaacggcacctataccacctttaccaccccgcatattccggcggtggcgaaacgctatgtgtatccgatgtgcgaacatggcattaaagcgagctattgcatggcgctgaacgatgcgatggtgagcgcgaacggcaacctgtatggcctggcggaaaaactgtttagcgaagatgaaggccagtgggaaaccaactattataaactgtattggagcaccggccagtggattatgagcatgaaatttattgaagaaagcattgataacgcgaacaacgattttgaaggctgcgataccggccattag

SEQ ID NO: 176 - protein, 362 aa, Saccharomyces cerevisiae
MKETTTSLMQDELTLGEPATQARMCVRLLRFFIGLTITAFIIAACIIKSATGGSGYSKAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYTTNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 177 - protein, 47 aa, Saccharomyces cerevisiae
MKETTTSLMQDELTLGEPATQARMCVRLLRFFIGLTITAFIIAACII

SEQ ID NO: 178 - protein, 140 aa, Saccharomyces cerevisiae
GGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIV

SEQ ID NO: 179 - protein, 94 aa, Saccharomyces cerevisiae
QDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 180 - DNA, 1089 bp, Saccharomyces cerevisiae
atgaaagaaaccaccaccagcctgatgcaggatgaactgaccctgggcgaaccggcgacccaggcgcgcatgtgcgtgcgcctgctgcgcttttttattggcctgaccattaccgcgtttattattgcggcgtgcattattaaaagcgcgaccggcggcagcggctatagcaaagcggtggcggtgcgcggcgaagcggataccccgagcaccattgtgggccagctggtggaacgcggcggctttcaggcgtgggcggtgggcgcgggcatttatctgtttgcgaaaattgcgtatgataccagcaaagtgaccgcggcggtgtgcaacccggaagcgctgattgcgattaccagctatgtggcgtatgcgccgaccctgtgcgcgggcgcgtatgtgattggcgcgatgagcggcgcgatgagcgcgggcctggcgctgtatgcgggctataaaggctggcagtggggcggcccgggcggcatggcggaacgcgaagatgtggcgagcttttatagcccgctgctgaacaacaccctgtatgtgggcggcgatcataccgcggattatgatagcgaactggcgaccattctgggcagcgtgtataacgatgtggtgcatctgggcgtgtattatgataacagcaccggcattgtgaaacgcgatagccgcccgagcatgattagctggaccgtgctgcatgataacatgatgattaccagctatcatcgcccggatcagctgggcgcggcggcgaccgcgtataaagcgtataccaccaacaccacccgcgtgggcaaacgccaggatggcgaatgggtgagctatagcgtgtatggcgaaaacgtggattatgaacgctatccggtggcgcatctgcaggaagaagcggatgcgtgctatgaaagcctgggcaacatgattaccagccaggtgcagccgtgcacccagcgcgaatgctatgcgatggatcagaaagtgtgcgcggcggtgggctttagcagcgatgcgggcgtgaacagcgcgatggtgggcgaagcgtatttttatgcgtatggcggcgtggatggcgaatgcgatagcggctaa

SEQ ID NO: 181 - DNA, 141 bp, Saccharomyces cerevisiae
atgaaagaaaccaccaccagcctgatgcaggatgaactgaccctgggcgaaccggcgacccaggcgcgcatgtgcgtgcgcctgctgcgcttttttattggcctgaccattaccgcgtttattattgcggcgtgcattatt

SEQ ID NO: 182 - DNA, 420 bp, Saccharomyces cerevisiae
ggcggctttcaggcgtgggcggtgggcgcgggcatttatctgtttgcgaaaattgcgtatgataccagcaaagtgaccgcggcggtgtgcaacccggaagcgctgattgcgattaccagctatgtggcgtatgcgccgaccctgtgcgcgggcgcgtatgtgattggcgcgatgagcggcgcgatgagcgcgggcctggcgctgtatgcgggctataaaggctggcagtggggcggcccgggcggcatggcggaacgcgaagatgtggcgagcttttatagcccgctgctgaacaacaccctgtatgtgggcggcgatcataccgcggattatgatagcgaactggcgaccattctgggcagcgtgtataacgatgtggtgcatctgggcgtgtattatgataacagcaccggcattgtg

SEQ ID NO: 183 - DNA, 285 bp, Saccharomyces cerevisiae
caggatggcgaatgggtgagctatagcgtgtatggcgaaaacgtggattatgaacgctatccggtggcgcatctgcaggaagaagcggatgcgtgctatgaaagcctgggcaacatgattaccagccaggtgcagccgtgcacccagcgcgaatgctatgcgatggatcagaaagtgtgcgcggcggtgggctttagcagcgatgcgggcgtgaacagcgcgatggtgggcgaagcgtatttttatgcgtatggcggcgtggatggcgaatgcgatagcggctaa

SEQ ID NO: 184 - protein, 345 aa, Saccharomyces cerevisiae
MESVSSLFNIFSTIMVNYKSLVLALLSVSNLKYARGMPTSERQQGLEERDFSAATCVLMGAEVGSWGMVYSGQKVESWILYVLTGITTMSAIVDEIDYYASHMPLSVVGENSGLQIVRDTIVTLVMAGLTASANKVISKTENAENIQSRSLIPGLLSMDYNSTHTMAINLEDVFSELGWDIDTSDSSGLYKRDDNSVTLHLGDVPALGTSNTIIPNAVMQIYNNASFAFGFAPHSNGNSTGLQKRASIDDAVWLQSAYGIAYSAWIGSENVGSYDEHLAEANGMANYWTSECSKYNGVIWGDESDACGNWLASQRLDIVSHSTGNYYRDVNLCGDDEARCHDELR

SEQ ID NO: 185 - protein, 48 aa, Saccharomyces cerevisiae
MESVSSLFNIFSTIMVNYKSLVLALLSVSNLKYARGMPTSERQQGLEE

SEQ ID NO: 186 - protein, 101 aa, Saccharomyces cerevisiae
RDFSAATCVLMGAEVGSWGMVYSGQKVESWILYVLTGITTMSAIVDEIDYYASHMPLSVVGENSGLQIVRDTIVTLVMAGLTASANKVISKTENAENIQSR

SEQ ID NO: 187 - protein, 196 aa, Saccharomyces cerevisiae
SLIPGLLSMDYNSTHTMAINLEDVFSELGWDIDTSDSSGLYKRDDNSVTLHLGDVPALGTSNTIIPNAVMQIYNNASFAFGFAPHSNGNSTGLQKRASIDDAVWLQSAYGIAYSAWIGSENVGSYDEHLAEANGMANYWTSECSKYNGVIWGDESDACGNWLASQRLDIVSHSTGNYYRDVNLCGDDEARCHDELR

SEQ ID NO: 188 - DNA, 1038 bp, Saccharomyces cerevisiae
atggaaagcgtgagcagcctgtttaacatttttagcaccattatggtgaactataaaagcctggtgctggcgctgctgagcgtgagcaacctgaaatatgcgcgcggcatgccgaccagcgaacgccagcagggcctggaagaacgcgattttagcgcggcgacctgcgtgctgatgggcgcggaagtgggcagctggggcatggtgtatagcggccagaaagtggaaagctggattctgtatgtgctgaccggcattaccaccatgagcgcgattgtggatgaaattgattattatgcgagccatatgccgctgagcgtggtgggcgaaaacagcggcctgcagattgtgcgcgataccattgtgaccctggtgatggcgggcctgaccgcgagcgcgaacaaagtgattagcaaaaccgaaaacgcggaaaacattcagagccgcagcctgattccgggcctgctgagcatggattataacagcacccataccatggcgattaacctggaagatgtgtttagcgaactgggctgggatattgataccagcgatagcagcggcctgtataaacgcgatgataacagcgtgaccctgcatctgggcgatgtgccggcgctgggcaccagcaacaccattattccgaacgcggtgatgcagatttataacaacgcgagctttgcgtttggctttgcgccgcatagcaacggcaacagcaccggcctgcagaaacgcgcgagcattgatgatgcggtgtggctgcagagcgcgtatggcattgcgtatagcgcgtggattggcagcgaaaacgtgggcagctatgatgaacatctggcggaagcgaacggcatggcgaactattggaccagcgaatgcagcaaatataacggcgtgatttggggcgatgaaagcgatgcgtgcggcaactggctggcgagccagcgcctggatattgtgagccatagcaccggcaactattatcgcgatgtgaacctgtgcggcgatgatgaagcgcgctgccatgatgaactgcgctaa

SEQ ID NO: 189 - DNA, 144 bp, Saccharomyces cerevisiae
atggaaagcgtgagcagcctgtttaacatttttagcaccattatggtgaactataaaagcctggtgctggcgctgctgagcgtgagcaacctgaaatatgcgcgcggcatgccgaccagcgaacgccagcagggcctggaagaa

SEQ ID NO: 190 - DNA, 303 bp, Saccharomyces cerevisiae
cgcgattttagcgcggcgacctgcgtgctgatgggcgcggaagtgggcagctggggcatggtgtatagcggccagaaagtggaaagctggattctgtatgtgctgaccggcattaccaccatgagcgcgattgtggatgaaattgattattatgcgagccatatgccgctgagcgtggtgggcgaaaacagcggcctgcagattgtgcgcgataccattgtgaccctggtgatggcgggcctgaccgcgagcgcgaacaaagtgattagcaaaaccgaaaacgcggaaaacattcagagccgc

SEQ ID NO: 191 - DNA, 591 bp, Saccharomyces cerevisiae
agcctgattccgggcctgctgagcatggattataacagcacccataccatggcgattaacctggaagatgtgtttagcgaactgggctgggatattgataccagcgatagcagcggcctgtataaacgcgatgataacagcgtgaccctgcatctgggcgatgtgccggcgctgggcaccagcaacaccattattccgaacgcggtgatgcagatttataacaacgcgagctttgcgtttggctttgcgccgcatagcaacggcaacagcaccggcctgcagaaacgcgcgagcattgatgatgcggtgtggctgcagagcgcgtatggcattgcgtatagcgcgtggattggcagcgaaaacgtgggcagctatgatgaacatctggcggaagcgaacggcatggcgaactattggaccagcgaatgcagcaaatataacggcgtgatttggggcgatgaaagcgatgcgtgcggcaactggctggcgagccagcgcctggatattgtgagccatagcaccggcaactattatcgcgatgtgaacctgtgcggcgatgatgaagcgcgctgccatgatgaactgcgctaa

SEQ ID NO: 192 - DNA, 8856 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgataccattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtccctggccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggactccaaaagctttcgagaagtcaacaggctcggctaatctttctaaaaccaaagttctggcacccgccagccgaagttcacatgctaacatcaaaccggtgggtcctgcaccagctataacaacatcgtacctcatgcggccgcttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgcggccggcctggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 193 - DNA, 1509 bp, Artificial Sequence
  Description of Artificial Sequence Synthetic polynucleotide
cattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtccctggccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggactccaaaagctttcgagaagtcaacaggctcggctaatctttctaaaaccaaagttctggcacccgccagccgaagttcacatgctaacatcaaaccggtgggtcctgcaccagctataacaacatcgtacctcat

SEQ ID NO: 194 - DNA, 8856 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgataccattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtccctggccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggaccgctagagctttcgagaagtcaacaggctcggctaatctttctaaaaccaaagttctggcacccgccagccgaagttcacatgctaacatcaaaccggtgggtcctgcaccagctataacaacatcgtacctcatgcggccgcttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgcggccggcctggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 195 - DNA, 1509 bp, Artificial Sequence
  Description of Artificial Sequence Synthetic polynucleotide
cattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtccctggccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggaccgctagagctttcgagaagtcaacaggctcggctaatctttctaaaaccaaagttctggcacccgccagccgaagttcacatgctaacatcaaaccggtgggtcctgcaccagctataacaacatcgtacctcat

SEQ ID NO: 196 - DNA, 8856 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgataccattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtcccaagccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggactcctagagctttcgagaagtcaacaggctcggctaatctttctaaaaccaaagttctggcacccgccagccgaagttcacatgctaacatcaaaccggtgggtcctgcaccagctataacaacatcgtacctcatgcggccgcttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgcggccggcctggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 197 - DNA, 1509 bp, Artificial Sequence
  Description of Artificial Sequence Synthetic polynucleotide
cattgcaggtgggccgaaccaagtacgcagcgcgtctgtcaatccatcagtaccagtttctggggcagctgtccaacaaatgtaaccatcaggccttacaagtaaagcagttaagccatctactgcagaaccagggtccgccacaccttcagctgcaactaaatcaaccctatctttccaagcagcggcttcatctgctagtggttgatcacaacctaacgttaataatacgccacgtgcagcatggaaataagtggacaactgtcttgttccatcttcggtgacaagaggtaagtcgggtactcttcttcccagcaatggatgttccccagccattgggtatctaacgtctaatgcggagattagccccgctaaatatctattagtctcaggaacgtgcaacaattcaatcattaattcccttaaaccctcgtagcgaggatctggatcaattagaacagcttgagcccttgtatttctcaaaacaccggcggctatcggccttctctcagcttcatacgtatccaataactctggtggagcggtcccggcaatggtagcaccaagcttccaacccaagttaacggcatcttgaaatcctaagttcagtcccaagccaccagcgggcaaatgaatatggcatgcatcaccagccaaaagaactctcccatctctatacctttctgcttgtctacttgtatcggtcaaacgagataaccatcttacatcatgcattccaaagtcagatccagcaacttctgttaacgcggctctgacatcctcctccgttaccggcgccctcctgtcagcatacggtctatcgaaaaaggcgactgttgctctgtagacgtctggttctagcggaaatgctgcgaaccatgcacgaaggtcatgtctcatgacaccataaggacgcataggacccataccttctccgtgaggaagctcttctctgaatcttgcgtctgcgatgacagcaaacatatgtgggtcttgacctggaaaatctatgcccaatagtttcctcaccgtactgcctccaccgtcgcagcccaccaaataagcacattctacttcgtatgggccttcagggcctgtcacgctcacagtaacaccatcagcatcttgttccaaggcggtcacggcatgaccacgacgcagctccgcccctagctccaaagctctacctgttagcagttcttcagttcgtacttgtggaacaaacaatgcatatgggtgtctagtatcaaatgatgagaaatccagggggacgcctaatgaggcaaaattaccacctcttaactttggtgcttcagcctggaatccttcaccgaggcctctcatatctaatagttcaacagtgcgagcgtggactcctagagctttcgagaagtcaacaggctcggctaatctttctaaaaccaaagttctggcacccgccagccgaagttcacatgctaacatcaaaccggtgggtcctgcaccagctataacaacatcgtacctcat

SEQ ID NO: 198 - DNA, 1509 bp, Artificial Sequence; identical to SEQ ID NO: 15
  Description of Artificial Sequence Synthetic polynucleotide

SEQ ID NO: 199 - DNA, 1974 bp, Artificial Sequence; identical to SEQ ID NO: 11
  Description of Artificial Sequence Synthetic polynucleotide

SEQ ID NO: 200 - DNA, 1681 bp, Artificial Sequence; identical to SEQ ID NO: 16
  Description of Artificial Sequence Synthetic polynucleotide

SEQ ID NO: 201 - DNA, 1602 bp, Artificial Sequence; identical to SEQ ID NO: 12
  Description of Artificial Sequence Synthetic polynucleotide

SEQ ID NO: 202 - DNA, 1309 bp, Artificial Sequence; identical to SEQ ID NO: 17
  Description of Artificial Sequence Synthetic polynucleotide

SEQ ID NO: 203 - DNA, 1674 bp, Artificial Sequence; identical to SEQ ID NO: 13
  Description of Artificial Sequence Synthetic polynucleotide

SEQ ID NO: 204 - DNA, 1381 bp, Artificial Sequence; identical to SEQ ID NO: 18
  Description of Artificial Sequence Synthetic polynucleotide

SEQ ID NO: 205 - DNA, 822 bp, Artificial Sequence
  Description of Artificial Sequence Synthetic polynucleotide
ccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgccattcactcaaaaggagatcacgtatttaagggctcaaggctacggccgactagccaccgtcggtgctcacggtgaacctcacaacgtgccggtatcttttgaaattgatgaagaaagaggtaccattgaaataacaggaagggatatgggacgttccagaaaattcagaaatgttgccaaaagtgacagagtggcatttgttgttgatgatgttccatgcagagacccagaagttgtcagagctgttgtaatacatggtactgcacaggcgcttcccacaggcggaagagaaaggcgtcctcattgtgctgacgaaatgattagaattcatccaagaagaatagtaacatggggtatcgaaggtgatttgtcaactggtgttcatgcaagagatattactgctgaagatggtggtagaagggtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttc

SEQ ID NO: 206 - DNA, 426 bp, Artificial Sequence; identical to SEQ ID NO: 59
  Description of Artificial Sequence Synthetic polynucleotide

SEQ ID NO: 207 - DNA, 816 bp, Artificial Sequence
  Description of Artificial Sequence Synthetic polynucleotide
ccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgccatttacagcaaaagaggctgcttatttggcctctcaaccactagccaggttggctacggtggatggtcgtggccagcctcacgtcgtgccgctagggttccattataacgcagaattgggcaccgtcgatgttactggtagaggtatggctagatccttaaagtacagacatgttcaaggtcatccaagagtatcattagtagttgacgatattgtagacgctcaaagatgggttgtccgtggaatagagatcaggggtactgctgttgcacttgccaccggaggaaaagaaattctgccacacgtagatgatgaattaatacgaatcacacctgaaagaattgttagttggggtattgacagcgattgtcaagccccccctctcgcgaggaatgtgggggcgggcgcacaggcagtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttc

SEQ ID NO: 208 - DNA, 420 bp, Artificial Sequence
  Description of Artificial Sequence Synthetic polynucleotide
atgccatttacagcaaaagaggctgcttatttggcctctcaaccactagccaggttggctacggtggatggtcgtggccagcctcacgtcgtgccgctagggttccattataacgcagaattgggcaccgtcgatgttactggtagaggtatggctagatccttaaagtacagacatgttcaaggtcatccaagagtatcattagtagttgacgatattgtagacgctcaaagatgggttgtccgtggaatagagatcaggggtactgctgttgcacttgccaccggaggaaaagaaattctgccacacgtagatgatgaattaatacgaatcacacctgaaagaattgttagttggggtattgacagcgattgtcaagccccccctctcgcgaggaatgtgggggcgggcgcacaggca

SEQ ID NO: 209 - DNA, 798 bp, Artificial Sequence
  Description of Artificial Sequence Synthetic polynucleotide
ccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaggatccatgtcattcactgccaaggaagttcaatatttgcgttctcagcaattaggaagattagccaccgttggtgctgatggtacaccacataatgttccagtaggctacagatacaacgctgacttggggaccattgacatcacaggaagggacttaagaagaagtaggaaatatagagatgttcaggctggctcgcgggtggcatttattgttgatgatctaccgagcacagcacctatagtcgcccgcgggctggaggtgaggggaacagctgaggcgctttctggtgaagaagatttgatacgtgtacgacctgcaagaatcgtcacttggggtattgaagcagattggcaagctggtcctactggtagaacggtaaggcccaccactccaagatccgcgacggtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttc

SEQ ID NO: 210 - DNA, 1548 bp, Streptomyces rimosus
atgaggtacgatgttgttatagctggtgcaggacccaccggtttgatgttagcatgtgaacttcggctggcgggtgccagaactttggttttagaaagattagccgagcctgttgacttctcgaaagctctaggagtccacgctcgcactgttgaactattagatatgagaggcctcggtgaaggattccaggctgaagcaccaaagttaagaggtggtaattttgcctcattaggcgtccccctggatttctcatcatttgatactagacacccatatgcattgtttgttccacaagtacgaactgaagaactgctaacaggtagagctttggagctaggggcggagctgcgtcgtggtcatgccgtgaccgccttggaacaagatgctgatggtgttactgtgagcgtgacaggccctgaaggcccatacgaagtagaatgtgcttatttggtgggctgcgacggtggaggcagtacggtgaggaaactattgggcatagattttccaggtcaagacccacatatgtttgctgtcatcgcagacgcaagattcagagaagagcttcctcacggagaaggtatgggtcctatgcgtccttatggtgtcatgagacatgaccttcgtgcatggttcgcagcatttccgctagaaccagacgtctacagagcaacagtcgcctttttcgatagaccgtatgctgacaggagggcgccggtaacggaggaggatgtcagagccgcgttaacagaagttgctggatctgactttggaatgcatgatgtaagatggttatctcgtttgaccgatacaagtagacaagcagaaaggtatagagatgggagagttcttttggctggtgatgcatgccatattcatttgcccgctggtggccagggactgaacttaggatttcaagatgccgttaacttgggttggaagcttggtgctaccattgccgggaccgctccaccagagttattggatacgtatgaagctgagagaaggccgatagccgccggtgttttgagaaatacaagggctcaagctgttctaattgatccagatcctcgctacgagggtttaagggaattaatgattgaattgttgcacgttcctgagactaatagatatttagcggggctaatctccgcattagacgttagatacccaatggctggggaacatccattgctgggaagaagagtacccgacttacctcttgtcaccgaagatggaacaagacagttgtccacttatttccatgctgcacgtggcgtattattaacgttaggttgtgatcaaccactagcagatgaagccgctgcttggaaagatagggttgatttagttgcagctgaaggtgtggcggaccctggttctgcagtagatggcttaactgctttacttgtaaggcctgatggttacatttgttggacagctgccccagaaactggtactgatggattgacagacgcgctgcgtacttggttcggcccacctgcaatggtatcgatggattacaaggatgacgacgataagatctga

SEQ ID NO: 211 - DNA, 1509 bp, Streptomyces rimosus; identical to SEQ ID NO: 15

SEQ ID NO: 212 - protein, 503 aa, Streptomyces rimosus
MRYDVVIAGAGPTGLMLACELRLAGARTLVLERLAEPVDFSKALGVHARTVELLDMRGLGEGFQAEAPKLRGGNFASLGVPLDFSSFDTRHPYALFVPQVRTEELLTGRALELGAELRRGHAVTALEQDADGVTVSVTGPEGPYEVECAYLVGCDGGGSTVRKLLGIDFPGQDPHMFAVIADARFREELPHGEGMGPMRPYGVMRHDLRAWFAAFPLEPDVYRATVAFFDRPYADRRAPVTEEDVRAALTEVAGSDFGMHDVRWLSRLTDTSRQAERYRDGRVLLAGDACHIHLPAGGQGLNLGFQDAVNLGWKLGATIAGTAPPELLDTYEAERRPIAAGVLRNTRAQAVLIDPDPRYEGLRELMIELLHVPETNRYLAGLISALDVRYPMAGEHPLLGRRVPDLPLVTEDGTRQLSTYFHAARGVLLTLGCDQPLADEAAAWKDRVDLVAAEGVADPGSAVDGLTALLVRPDGYICWTAAPETGTDGLTDALRTWFGPPAM

SEQ ID NO: 213 - protein, 515 aa, Artificial Sequence
  Description of Artificial Sequence Synthetic polypeptide
MRYDVVIAGAGPTGLMLACELRLAGARTLVLERLAEPVDFSKAFGVHARTVELLDMRGLGEGFQAEAPKLRGGNFASLGVPLDFSSFDTRHPYALFVPQVRTEELLTGRALELGAELRRGHAVTALEQDADGVTVSVTGPEGPYEVECAYLVGCDGGGSTVRKLLGIDFPGQDPHMFAVIADARFREELPHGEGMGPMRPYGVMRHDLRAWFAAFPLEPDVYRATVAFFDRPYADRRAPVTEEDVRAALTEVAGSDFGMHDVRWLSRLTDTSRQAERYRDGRVLLAGDACHIHLPAGGQGLNLGFQDAVNLGWKLGATIAGTAPPELLDTYEAERRPIAAGVLRNTRAQAVLIDPDPRYEGLRELMIELLHVPETNRYLAGLISALDVRYPMAGEHPLLGRRVPDLPLVTEDGTRQLSTYFHAARGVLLTLGCDQPLADEAAAWKDRVDLVAAEGVADPGSAVDGLTALLVRPDGYICWTAAPETGTDGLTDALRTWFGPPAMVSMDYKDDDDKI

SEQ ID NO: 214 - protein, 515 aa, Artificial Sequence
  Description of Artificial Sequence Synthetic polypeptide
MRYDVVIAGAGPTGLMLACELRLAGARTLVLERLAEPVDFSKALAVHARTVELLDMRGLGEGFQAEAPKLRGGNFASLGVPLDFSSFDTRHPYALFVPQVRTEELLTGRALELGAELRRGHAVTALEQDADGVTVSVTGPEGPYEVECAYLVGCDGGGSTVRKLLGIDFPGQDPHMFAVIADARFREELPHGEGMGPMRPYGVMRHDLRAWFAAFPLEPDVYRATVAFFDRPYADRRAPVTEEDVRAALTEVAGSDFGMHDVRWLSRLTDTSRQAERYRDGRVLLAGDACHIHLPAGGQGLNLGFQDAVNLGWKLGATIAGTAPPELLDTYEAERRPIAAGVLRNTRAQAVLIDPDPRYEGLRELMIELLHVPETNRYLAGLISALDVRYPMAGEHPLLGRRVPDLPLVTEDGTRQLSTYFHAARGVLLTLGCDQPLADEAAAWKDRVDLVAAEGVADPGSAVDGLTALLVRPDGYICWTAAPETGTDGLTDALRTWFGPPAMVSMDYKDDDDKI

SEQ ID NO: 215 - protein, 515 aa, Artificial Sequence
  Description of Artificial Sequence Synthetic polypeptide
MRYDVVIAGAGPTGLMLACELRLAGARTLVLERLAEPVDFSKALGVHARTVELLDMRGLGEGFQAEAPKLRGGNFASLGVPLDFSSFDTRHPYALFVPQVRTEELLTGRALELGAELRRGHAVTALEQDADGVTVSVTGPEGPYEVECAYLVGCDGGGSTVRKLLGIDFPGQDPHMFAVIADARFREELPHGEGMGPMRPYGVMRHDLRAWFAAFPLEPDVYRATVAFFDRPYADRRAPVTEEDVRAALTEVAGSDFGMHDVRWLSRLTDTSRQAERYRDGRVLLAGDACHIHLPAGGLGLNLGFQDAVNLGWKLGATIAGTAPPELLDTYEAERRPIAAGVLRNTRAQAVLIDPDPRYEGLRELMIELLHVPETNRYLAGLISALDVRYPMAGEHPLLGRRVPDLPLVTEDGTRQLSTYFHAARGVLLTLGCDQPLADEAAAWKDRVDLVAAEGVADPGSAVDGLTALLVRPDGYICWTAAPETGTDGLTDALRTWFGPPAMVSMDYKDDDDKI

SEQ ID NO: 216 - DNA, 20 bp, Mycobacterium tuberculosis
cattgaacaacacccggttt

(19) United States
(12) Patent Application Publication
(10) Pub. No.: US 2023/0000934 A1
(43) Pub. Date: Jan. 5, 2023

(54) METHODS FOR ENGINEERING THERAPEUTICS AND USES THEREOF

(71) Applicant: The Trustees of Columbia University In The City of New York, New York, NY (US)

(72) Inventor: Virginia CORNISH, New York, NY (US)

(73) Assignee: THE TRUSTEES OF COLUMBIA UNIVERSITY IN THE CITY OF NEW YORK, New York, NY (US)

(21) Appl. No.: 17/738,799

(22) Filed: May 6, 2022

Related U.S. Application Data
(63) Continuation of application No. PCT/US2020/059747, filed on Nov. 9, 2020.
(60) Provisional application No. 62/933,249, filed on Nov. 8, 2019.

(51) Int. Cl. A61K 36/064; C12N 15/81; C12P 29/00; C12P 21/00; A61P 31/04; C07K 14/395; C12N 15/52

(52) U.S. Cl. CPC A61K 36/064; C12N 15/81; C12P 29/00; C12P 21/00; A61P 31/04; C07K 14/395; C12N 15/52

(57) ABSTRACT

The disclosed subject matter provides for genetically modified cells, e.g., fungal cells, that autonomously generates and/or secretes one or more therapeutic molecules, e.g., therapeutic peptides, therapeutic proteins or small therapeutic molecules, in situ. In certain embodiments, the present disclosure provides genetically-engineered fungal cells that generate and secrete tetracycline and analogues thereof.

### CROSS-REFERENCE TO RELATED APPLICATIONS

This application is a continuation of International Patent Application No. PCT/US2020/059747 filed Nov. 9, 2020, which claims priority to U.S. Provisional Patent Application No. 62/933,249, filed on Nov. 8, 2019, the contents of which are incorporated by reference in their entirety, and to which priority is claimed.

### SEQUENCE LISTING

The instant application contains a Sequence Listing which has been submitted electronically in ASCII format and is hereby incorporated by reference in its entirety. Said ASCII copy, created on May 6, 2022, is named 070050_6620_SL.txt and is 340,967 bytes in size.

### TECHNICAL FIELD

The present disclosure relates to genetically-engineered fungal cells for the generation of therapeutic molecules and analogues thereof, and methods of treating a subject in need thereof by administering such fungal cells.

### BACKGROUND

Small molecule therapeutics as well as proteins and peptides have been used to treat conditions. However, procedures for synthesizing and isolating these molecules can be complex and require substantive resources. Genetically engineered cells through various mechanisms are capable of producing some small molecule therapeutics as well as proteins and peptides. However, long and cost inefficient procedures are often required to isolate the molecule of interest from the genetically modified cell culture. Additionally, certain therapeutic molecules degrade rapidly and are negatively affected by purification processes.

Therefore, there is a need in the art for improved methods for developing, synthesizing and administering various therapeutic molecules.

### SUMMARY

The disclosed subject matter provides for genetically-engineered cells, e.g., genetically-engineered fungal cells, that autonomously generates and/or secretes one or more therapeutic compounds. The present disclosure further provides pharmaceutical compositions including the disclosed genetically-engineered cells and methods of administering the disclosed genetically-engineered cells for treating a subject in need thereof. The present disclosure provides a fungal cell genetically engineered to produce a therapeutic molecule in situ, wherein the therapeutic molecule is secreted from the fungal cell. In certain embodiments, the therapeutic molecule is secreted from the fungal cell by a secretory pathway of the fungal cell. In certain embodiments, the fungal cell expresses a heterologous efflux pump, e.g., for secretion of the therapeutic molecule. In certain embodiments, the genetically-engineered fungal cell secretes multiple therapeutic molecules, e.g., two or more, three or more, four or more, five or more or six or more therapeutic molecules.

In certain embodiments, the therapeutic molecule is selected from the group consisting of a peptide, a small molecule and a combination thereof. In certain embodiments, the therapeutic molecule is a small molecule. In certain embodiments, the small molecule has anti-inflammatory and/or antibiotic properties. In certain embodiments, the small molecule is used to treat an infection selected from the group consisting of intraabdominal infections, respiratory infections, bacterial infections, urinary tract infections, urethral infections, cervical infections and rectal infections. In certain embodiments, the small molecule is TAN-1612 or a derivative thereof.

In certain embodiments, the genetically-engineered fungal cell heterologously expresses a protein involved in the biosynthesis pathway of the therapeutic molecule. In certain embodiments, the protein involved in the biosynthesis pathway of the therapeutic molecule is an enzyme. Non-limiting examples of such enzymes include a transferase, a synthase, a lactamase, a monooxygenase, a reductase, a hydroxylase, an oxidoreductase, a glycotransferase and a fusion protein thereof.

In certain embodiments, the genetically-engineered fungal cell expresses an enzyme selected from the group consisting of AdaA, AdaB, AdaC, AdaD, NpgA and a combination thereof for synthesizing TAN-1612. For example, but not by way of limitation, the genetically-engineered fungal cell expresses all five of AdaA, AdaB, AdaC, AdaD and NpgA.

In certain embodiments, the genetically-engineered fungal cell further expresses an enzyme for modifying TAN-1612 to synthesize a TAN-1612 analogue. In certain embodiments, the enzyme is a monooxygenase, a reductase, a hydroxylase, an oxidoreductase, a glycotransferase, a fusion protein thereof and a combination thereof. For example, but not by way of limitation, the enzyme is selected from the group consisting of PgaE, DacO1, DacO4, PgaE, SsfO1, CtcN, CtcM, FNO, OxyS and a combination thereof. In certain embodiments, directed evolution is used to modify the enzyme to accept TAN-1612 as a substrate. In certain embodiments, OxyS is a OxyS mutant that has one or more mutations at amino acids K42, A43, L44, G45, L95, F96, M176, W211, F212, T225, A227, F228, V240, P295, A296, G297, G298, G299, N302, I353, D354, R358, V372, P375 or a combination thereof.

In certain embodiments, the genetically-engineered fungal cell further expresses an enzyme for modifying TAN-1612 to synthesize tetracycline or an analogue thereof. In certain embodiments, the enzyme is a monooxygenase, a reductase, a hydroxylase, an oxidoreductase, a glycotransferase, a fusion protein thereof and a combination thereof. For example, but not by way of limitation, the enzyme can be OxyS, CtcM, FNO and a combination thereof.

In certain embodiments, the therapeutic molecule is a peptide. In certain embodiments, the peptide has anti-fungal and/or antibiotic properties. In certain embodiments, the peptide is a toxin peptide. In certain embodiments, the toxin peptide is derived from a fungal cell. In certain embodiments, the toxin peptide is a K1, K2 or K28 toxin peptide derived from Saccharomyces cerevisiae.

The present disclosure further provides for methods of treating a subject in need thereof. In certain embodiments, a method of the present disclosure includes administering to the subject a fungal cell genetically engineered to generate and secrete a therapeutic molecule in situ for treating the subject. In certain embodiments, the therapeutic molecule is secreted from the genetically-engineered fungal cell by a secretory pathway of the genetically engineered fungal cell. In certain embodiments, the genetically-engineered fungal cell expresses a heterologous efflux pump. In certain embodiments, the genetically-engineered fungal cell is a live genetically-engineered fungal cell. In certain embodiments, the genetically-engineered fungal cell secretes multiple therapeutic molecules, e.g., two or more, three or more, four or more, five or more or six or more therapeutic molecules.

In certain embodiments, the therapeutic molecule secreted by the genetically-engineered cells administered according to the disclosed methods is selected from the group consisting a peptide, a small molecule and a combination thereof. In certain embodiments, the therapeutic molecule is a small molecule. In certain embodiments, the small molecule has anti-inflammatory and/or antibiotic properties. In certain embodiments, the small molecule is used to treat an infection selected from the group consisting of intraabdominal infections, respiratory infections, bacterial infections, urinary tract infections, urethral infections, cervical infections and rectal infections. In certain embodiments, the small molecule administered in a disclosed method is TAN-1612 or a derivative thereof.

In certain embodiments, the genetically-engineered fungal cell administered according to the disclosed methods heterologously expresses a protein involved in the biosynthesis pathway of the therapeutic molecule. In certain embodiments, the protein involved in the biosynthesis pathway of the therapeutic molecule is an enzyme. In certain embodiments, the enzyme is selected from the group consisting of a transferase, a synthase, a lactamase, a monooxygenase, a reductase, a hydroxylase, an oxidoreductase, a glycotransferase, a fusion protein thereof and a combination thereof. In certain embodiments, the enzyme is selected from the group consisting of AdaA, AdaB, AdaC, AdaD, NpgA and a combination thereof.

In certain embodiments, a genetically-engineered fungal cell administered according to the disclosed methods further expresses an enzyme for modifying TAN-1612 to synthesize a TAN-1612 analogue. In certain embodiments, the enzyme is a monooxygenase, a reductase, a hydroxylase, an oxidoreductase, a glycotransferase, a fusion protein thereof and a combination thereof. In certain embodiments, the enzyme for modifying TAN-1612 is selected from the group consisting of consisting of PgaE, DacO1, DacO4, PgaE, SsfO1, CtcN, CtcM, FNO, OxyS and a combination thereof. In certain embodiments, directed evolution is used to modify the enzyme to accept TAN-1612 as a substrate. In certain embodiments, the OxyS is a OxyS mutant that includes one or more mutations at amino acids K42, A43, L44, G45, L95, F96, M176, W211, F212, T225, A227, F228, V240, P295, A296, G297, G298, G299, N302, I353, D354, R358, V372, P375 or a combination thereof.

In certain embodiments, a genetically-engineered fungal cell administered according to the disclosed methods further expresses an enzyme for modifying TAN-1612 to synthesize tetracycline or an analogue thereof. In certain embodiments, the enzyme is a monooxygenase, a reductase, a hydroxylase, an oxidoreductase, a glycotransferase, a fusion protein thereof and a combination thereof. In certain embodiments, the enzyme can be OxyS, CtcM, FNO and a combination thereof.

In certain embodiments, the therapeutic molecule secreted by the genetically-engineered cells administered according to the disclosed methods is a peptide. In certain embodiments, the peptide is a fungal toxin peptide. In certain embodiments, the fungal toxin peptide is a K1, K2 or K28 toxin peptide derived from Saccharomyces cerevisiae.

In certain embodiments, the genetically-engineered fungal cell administered according to the disclosed methods is formulated for parenteral administration, intraocular administration, intraaural administration, intranasal administration, oral administration, rectal administration, vaginal administration or topical administration. In certain embodiments, the genetically-engineered fungal cell is not administered to the digestive system. In certain embodiments, the genetically-engineered fungal cell is administered to the subject to treat an infection.

In certain embodiments, a genetically-engineered fungal cell of the present disclosure is one or more species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces and a combination thereof. In certain embodiments, the fungal cell is Saccharomyces cerevisiae or Saccharomyces boulardii.

The present disclosure further provides a pharmaceutical composition that includes one or more genetically-engineered fungal cells disclosed herein and a pharmaceutically acceptable carrier. In certain embodiments, the pharmaceutical composition is formulated for parenteral administration, intraocular administration, intraaural administration, intranasal administration, oral administration, rectal administration, vaginal administration or topical administration.

The present disclosure further provides an OxyS protein with one or more mutations. For example, but not by way of limitation, an OxyS protein of the present disclosure is mutated at one or more amino acid selected from the group consisting of K42, A43, L44, G45, L95, F96, M176, W211, F212, T225, A227, F228, V240, P295, A296, G297, G298, G299, N302, I353, D354, R358, V372, P375 and a combination thereof.

### BRIEF DESCRIPTION OF THE DRAWINGS

FIG. 1 illustrates a composition of the present disclosure and application thereof.

FIG. 2 provides a two-part process for enzymatic conversion of anhydrotetracycline to tetracycline.

FIGS. 3A-3B provide a mass spectrometry analysis of anhydrotetracycline hydroxylation in cell lysate expressing OxyS. Cell lysates of an OxyS-expressing strain EH-3-248-1 (+OxyS) or a no-hydroxylase control EH-3-248-8 (−OxyS) were placed overnight in TRIS buffer (100 mM, pH 7.45) containing anhydrotetracycline (5.4 mM), glucose (27.8 mM) and NADPH (3 mM) mercaptoethanol (18.5 mM). After overnight incubation, methanol was added, the contents were mixed and the reaction was filtered prior to MS analysis. m/z calculation for protonated anhydrotetracycline (C₂₂H₂₃N₂O₇+), 427.15; found, 427.3 [M+H]+. m/z calculation for protonated 5a(11a)-dehydrotetracycline and for protonated 5(5a)-dehydrotetracycline (2a and 2b, respectively, C₂₂H₂₃N₂O₈+), 443.15; found, 443.3 [M+H]+. The expected [M+H]+value for 2 holds for either 2a or 2b as the two are isomers (Scheme 1). Mass spectra are shown in FIG. 3B and ion counts for certain peaks are provided in FIG. 3A.

FIGS. 4A-4C provide a UV/Vis spectroscopy analysis of the reaction of anhydrotetracycline in whole cells expressing OxyS. The spectra shown are reduction spectra, that is, the absorption/emission values for the OxyS expressing cells EH-98-6 minus the absorption/emission values of the no hydroxylase control EH-3-80-3 are shown.

FIGS. 5A-5B provide a mass spectrometry analysis of anhydrotetracycline hydroxylation and reduction in cell lysate expressing OxyS. Cell lysate of OxyS expressing strain EH-3-248-1 was placed overnight in TRIS buffer (100 mM, pH 7.45) containing anhydrotetracycline (5.4 mM), glucose (27.8 mM), NADPH (3 mM), 10 mM glucose-6-phosphate (+G6P) or 0 mM glucose-6-phosphate (−G6P) and mercaptoethanol (18.5 mM). Mass spectra are shown in FIG. 5B and ion counts for certain peaks are provided in FIG. 5A.

FIGS. 6A-6B provide a western blot of DacO1 and OxyS. Cultures of DacO1, DacO4 and OxyS, OxyR encoding strains EH-3-153-7 and EH-3-153-8, respectively, were lysed with Y-PER and labeled with Monoclonal ANTI-FLAG HRP antibody. Two biological replicates were used for each strain indicated as colony 1 and 2 (C1 and C2), respectively. The expected sizes are 55.1, 55.9, 16.3 and 17.5 for DacO1, OxyS, DacO4 and OxyR, respectively. DacO1 and OxyS is shown in FIG. 6A and DacO4 and OxyR are shown in FIG. 6B.

FIG. 7 shows bacterial monooxygenases and their corresponding substrates and products. Bacterial flavin-dependent monooxygenases OxyS, DacO1, SsfO1, CtcN and PgaE and their native substrates. Marked in red are differences between the native substrate and anhydrotetracycline.

FIG. 8 depicts a microtiter plate assay for anhydrotetracycline hydroxylation.

FIGS. 9A-9B provide a DacO1 error-prone mutagenesis screen excerpt using the microtiter plate assay for anhydrotetracycline hydroxylation where FIG. 9A depicts a plate with only the OxyS positive control significantly above background fluorescence. FIG. 9B depicts a plate with both the OxyS positive control and a DacO1 mutant hit with fluorescence significantly above background. Wells 60 and 72 contain the positive control OxyS encoding strain EH-3-98-6. λex=400 nm.

FIG. 10 provides a ΔExcitation spectrum for DacO1 and DacO1 error-prone PCR mutant. The spectrum shown is an A spectrum, that is, the values shown are the emission values for the hydroxylase expressing cells minus the emission values of the no hydroxylase control EH-3-80-3. λemission=500 nm. Each value is the average of six biological replicates and the error bars represent standard error.

FIG. 11 provides a western blot analysis of DacO1 fusion proteins and other bacterial hydroxylases in FY251. Strain cultures were lysed with Y-PER and labeled with monoclonal ANTI-FLAG HRP antibody. Two biological replicates were used for each strain indicated as colony 1 and 2 (C1 and C2), respectively. Strains EH-5-98-1, EH-5-98-3, EH-5-98-9, EH-5-98-10 and EH-5-98-13 encode DacO1 fusions with ubiquitin, Gall, Ubiquitin-γ-IFN, γ-IFN, and OxyS (first 37 amino acids), respectively. In the latter only the last 461 amino acids of DacO1 are encoded, while in the other DacO1 fusion proteins the complete DacO1 protein is encoded. Strains EH-3-80-2, EH-3-80-3 and EH-3-98-6 encode DacO1, no hydroxylase and OxyS, respectively. The size (kDa) indicated below the gels is the expected size of the protein based on the amino acid sequence.

FIG. 12 provides a western blot analysis of bacterial hydroxylases in BJ-5464-NpgA. Strain cultures were lysed with Y-PER and labeled with monoclonal ANTI-FLAG HRP antibody. Two biological replicates were used for each strain indicated as colony 1 and 2 (C1 and C2), respectively. The size (kDa) indicated below the gels is the expected size of the protein based on the amino acid sequence.

FIG. 13 provides a western blot analysis of DacO1-DacO4 and DacO4-DacO1 fusion proteins. Strain cultures were lysed with Y-PER and labeled with monoclonal ANTI-Myc HRP antibody. Two biological replicates were used for each strain indicated as colony 1 and 2 (C1 and C2), respectively. The size (kDa) indicated below the gels is the expected size of the protein based on the amino acid sequence.

FIG. 14 provides a western blot analysis of DacO1 and its fusion proteins in BJ5464-NpgA. Strain cultures were lysed with Y-PER and labeled with monoclonal ANTI-FLAG HRP antibody. Two biological replicates were used for each strain indicated as colony 1 and 2 (C1 and C2), respectively. Strains EH-5-163-1, through EH-5-163-7 encode DacO1 fusions with ubiquitin, Gall, Ubiquitin-γ-IFN, γ-IFN, DacO4, OxyS (first 37 amino acids) and OxyS (first 87 amino acids), respectively. In the last two strains only the last 461 and 411 amino acids of DacO1 are encoded, respectively, while the other DacO1 fusion proteins the complete DacO1 protein is encoded. In contrast to DacO1 and its fusion proteins, PgaE expressing strain in this assay has an FY251 background. The size (kDa) indicated below the gels is the expected size of the protein based on the amino acid sequence.

FIG. 15 provides an example process for enzymatic conversion of (6-demethyl-) anhydrotetracycline to (6-demethyl-)6-epitetracycline by a hydroxylase (e.g., DacO1) and reductase (e.g., DacO4).

FIG. 16 provides a mass spectrometry analysis of anhydrotetracycline hydroxylation and reduction by cell lysates of strains expressing DacO1, its fusion proteins and other bacterial hydroxylases. The analyzed strains shown are EH-3-248-1, EH-3-248-4, EH-3-248-6, EH-3-248-7, EH-3-248-8, EH-5163-1-C1, EH-5-163-2-C1, EH-5-163-2-C2, EH-5-163-3-C2, EH-5-163-4-C2, EH-5-163-6-C1 and EH-5-163-7-C1 encoding hydroxylases OxyS, DacO1, SsfO1, PgaE, pSPG1 (no hydroxylase negative control), Ubiquitin-DacO1, Gall-DacO1, Gall-DacO1, Ubiquitin-γ-IFN-DacO1, γ-IFN-DacO1, OxyS-DacO1-37 and OxyS-DacO1-87, respectively. Cell lysates were placed overnight in TRIS buffer (100 mM, pH 7.45) containing anhydrotetracycline (5.4 mM), glucose (27.8 mM), NADPH (3 mM) 10 mM glucose-6-phosphate (+G6P) and mercaptoethanol (18.5 mM).

FIG. 17 provides a mass spectrometry analysis of anhydrotetracycline hydroxylation and reduction in cell lysates of strains expressing DacO1, its fusion proteins and other bacterial hydroxylases. The analyzed strains shown EH-3-248-1, EH-3-248-4, EH-3-248-7, EH-3-248-8, EH-5-163-3-C2, EH-5163-4-C2, EH-5-163-6-C1, EH-5-163-7-C1 are encoding hydroxylases OxyS, DacO1, PgaE, pSPG1 (no hydroxylase negative control), Ubiquitin-γ-IFN-DacO1, γ-IFN-DacO1, OxyS-DacO1-37 and OxyS-DacO1-87, respectively.

FIG. 18 provides a mass spectrometry analysis of anhydrotetracycline hydroxylation and reduction in cell lysates of strains expressing OxyS and PgaE. The analyzed strains shown are EH-3-248-1 and EH-3-248-7 encoding hydroxylases OxyS and PgaE, respectively, in condition A (Table 8). TIC is total ion count. 443 and 445 refer to ion counts of these m/z values.

FIG. 19 shows a biosynthetic plan for TAN-1612 6α-hydroxylation.

FIG. 20 shows bacterial monooxygenases and their corresponding substrates and products. Bacterial flavin-dependent monooxygenases OxyS, DacO1, SsfO1, CtcN and PgaE and their native substrates. Marked in blue are differences between the native substrate and TAN-1612.

FIGS. 21A-21B provide a UV/Vis analysis of hydroxylation attempt of TAN-1612 by bacterial hydroxylases. FIG. 21A depicts excitation spectrum (λemission=560 nm) and FIG. 21B depicts emission spectrum (λexcitation=400 nm) of strains EH-5-2121, EH-5-212-2, EH-5-212-3 and EH-5-212-4 harboring a plasmid encoding the TAN-1612 pathway as well as a plasmid encoding OxyS, PgaE, SsfO1 and no hydroxylase, respectively. Emission and excitation spectra were taken after diluting cultures that were incubated for 3 nights in UT-media (4 mL) in 15 mL culture tubes (Corning 352059). Each data point and error bar represent the average and standard error of three biological replicates, respectively.

FIG. 22 provides a UV/Vis chromatogram of the HPLC separation of extracts from +/− PgaE strains encoding the TAN-1612 pathway. Strains EH-5-212-2 and EH-5-212-4 harboring a plasmid encoding the TAN-1612 pathway as well as a plasmid encoding PgaE and no hydroxylase, respectively, are indicated as +PgaE and −PgaE, respectively. Both strains were cultured in 500 mL scale for three nights. Prior to HPLC separation the culture was extracted twice with EtOAc and the combined organic extract was washed with H₂O and dried with Na₂SO₄, with the organic solvent was removed under reduced pressure. Absorption chromatograms are shown for 254 and 400 nm for an HPLC separation method of 10:90 to 90:10 MeCN in 99.9% H2O/0.1% TFA over 1 h.

FIGS. 23A-23B provide a mass spectrometry analysis of isolate from +/−PgaE strain encoding the TAN-1612 pathway. Liquid chromatography mass spectrometry analyses for isolates from strains EH-5-212-2 and EH-5-212-4 harboring a plasmid encoding the TAN-1612 pathway as well as a plasmid encoding PgaE and no hydroxylase, are shown in FIG. 23B and FIG. 23A, respectively. Both strains were cultured in 500 mL scale for three nights. Prior to HPLC separation the culture was extracted twice with EtOAc and the combined organic extract was washed with H₂O and dried with Na₂SO₄, with the organic solvent was removed under reduced pressure. The LCMS separation method is 5:95 to 95:5 MeCN in 99.9% H2O/0.1% formic acid over 2 min.

FIG. 24 provides an MS-MS analysis of isolate from +PgaE strain encoding the TAN-1612 pathway. Liquid chromatography MSMS analyses for isolate from strain EH-5-212-2 harboring a plasmid encoding the TAN-1612 pathway as well as a plasmid encoding PgaE with ion selection at mass 593. The LC-MS-MS separation method is 5:95 to 95:5 MeCN in 99.9% H₂O/0.1% formic acid over 2 min.

FIG. 25 provides ¹H-NMR spectra of isolates from +/−PgaE strains encoding the TAN-1612 pathway. ¹H-NMR spectra of isolates from strains EH-5-212-2 and EH-5-212-4 harboring a plasmid encoding the TAN-1612 pathway as well as a plasmid encoding PgaE and no hydroxylase, are shown in (B) and (A), respectively. Both strains were cultured in 500 mL scale for three nights. Prior to HPLC separation the culture was extracted twice with EtOAc and the combined organic extract was washed with H₂O and dried with Na₂SO₄, with the organic solvent was removed under reduced pressure. NMR spectra shown are in MeOD-d4 for the major product of each strain according to HPLC chromatogram. ¹H NMR (for (b), δ>4 ppm, 500 MHz, MeOD-d4): δ 7.61 (dd, J=8.0, 1.0, 1H), 7.47 (d, J=8.6, 2H), 7.22 (dd, J=8.0, 1.1, 1H), 6.88 (dd, J=8.6, 2H), 6.85 (dd, J=8.0, 1H), 6.72 (s, 1H).

FIG. 26 provides a COSY NMR spectrum of isolate from +PgaE strain encoding the TAN-1612 pathway. Strain EH-5-212-2 harboring a plasmid encoding the TAN-1612 pathway as well as a plasmid encoding PgaE. Both strains were cultured in 500 mL scale for three nights. Prior to HPLC separation the culture was extracted twice with EtOAc and the combined organic extract was washed with H2O and dried with Na2SO4, with the organic solvent was removed under reduced pressure. NMR spectra shown is in MeOD-d4 for the major product according to the HPLC chromatogram (FIG. 21).

FIG. 27 provides a western blot analysis of fungal hydroxylase expression in BJ5464-NpgA. Strain cultures were lysed with Y-PER and labeled with monoclonal ANTI-FLAG HRP antibody. The size (kDa) indicated below the gels is the expected size of the protein based on the amino acid sequence. Strains EH-5-227-1 through EH-5-227-9 encode fungal monooxygenase Entry 6, 5, 1, 4, 2, 2, 8, 11, 12, from Table 17, respectively.

FIGS. 28A-28B provide a PyMOL illustrations of amino acids in proximity to the tetracycline substrate in aklavinone-11-Hydroxylase and in OxyS. FIG. 28A shows a PyMOL illustration of alkavinone and FAD (stick representation, carbons colored white) surrounded by amino acids of aklavinone-11-Hydroxylase within 5 Å of alkavinone (stick representation, carbons colored green, PDB ID 3IHG) and FIG. 28B shows a PyMOL illustration of FAD (carbons colored white) surrounded by OxyS (surface representation, carbons colored green, PDB ID 4K2X) and its amino acids that are homologous to those of aklavinone-11-Hydroxylase amino acids that are within 5 Å of alkavinone in the structure shown in FIG. 28A.

FIG. 29 provides a library screening for TAN-1612 hydroxylation in S. cerevisiae. Y axes shows the sum of absorption at 400 and 450 nm divided by absorption at 600 nm while the X axes show the colony number. Plates a, b and c are screens of strain library EH-5-217-2. Plates d, e and f are screens of strain library EH-5-217-4. Both strain libraries EH-5-2172 and EH-5-217-4 encode the same OxyS saturation mutagenesis library and differ in the background TAN-1612 producing strain. Plate g contains strain libraries EH-5-217-1 and EH-5-217-3 encoding bacterial and fungal hydroxylases, including selected DacO1 fusion proteins.

FIGS. 30A-30C provide possible moieties in the product isolated from +PgaE strain co-expressing the TAN-1612 biosynthetic pathway. Either of the three aromatic moieties of FIG. 30A can be responsible for generating the protons of chemical shifts 7.61, 7.22 and 6.85 ppm, the aromatic moiety of FIG. 30B can be responsible for generating protons of chemical shifts 7.47 and 6.88 ppm and the aromatic moiety of FIG. 30C can be responsible for generating a proton of chemical shifts 6.72 ppm in the 1H-NMR spectrum of the product isolated from +PgaE strain co-expressing the TAN-1612 biosynthetic pathway (FIG. 24). The squiggly lines represent any non-proton substituent.

FIG. 31 provides possible intermediates in the biosynthesis of TAN-1612. TAN-1612 and its intermediates 2-10 that can occur in the case of exclusion or disfunction of AdaB, AdaC or AdaD, the three post PKS biosynthetic enzymes of the TAN-1612 biosynthetic pathway, or combinations thereof.

FIG. 32 provides xanthurenic acid and its derivative that can correspond to experimental mass and NMR spectra. The xanthurenic acid derivatives presented in this figure have [M+H]⁺ values of 593.1196, 298.0715 and 296.0559 that are 0.0022, 0.0028 and 0.0008 amu from the experimental values detected 593.1218, 298.0743 and 296.0551 (FIG. 22 and FIG. 23). In addition, the protons of a molecule such as the one shown with exact mass C16H11NO5 can correspond to the 6 proton types identified in the NMR spectrum (FIG. 24 and FIG. 25) and identified to potentially belong to the moieties described in FIG. 29.

FIGS. 33A-33C. FIG. 33A provides three new classes of Tc analogs can be accessed both purely biosynthetically and by semisynthesis starting from TAN-1612. FIG. 33B shows biosynthetic (parts 1-5) and chemical (part 6) conversion of TAN-1612 into 6α-analogs, 4α-analogs and 6α-4α-analogs. The employed heterologous enzymes are indicated. FIG. 33C provides several examples of glycoside modifications for the glycoside library.

FIG. 34 depicts key FDA approved tetracycline natural products.

FIG. 35 provides new classes of Tc analogs. Three new classes of Tc analogs can be accessed both purely biosynthetically and by semisynthesis starting from TAN-1612.

FIG. 36 provides a process to synthesize glycotetracyclines from TAN-1612.

FIG. 37 illustrates isolation of TAN-1612 from A. niger.

FIGS. 38A-38C provide the growth of A. niger after inoculation (FIG. 38A), comparison of plate undersides (FIG. 38B) and extracts after subsequent washes (FIG. 38C).

FIGS. 39A-39B provide mass spectrum analysis after column chromatography (FIG. 39A), and pTLC plates analysis of chromatography purification (FIG. 39B).

FIGS. 40A-40B provide the chemical structure of TAN-1612 (FIG. 40A), and nuclear magnetic resonance (NMR) spectrums analysis of isolated TAN-1612 from A. niger (FIG. 40B).

FIGS. 41A-41B provide the chemical structure of TAN-1612 (FIG. 41A), and nuclear magnetic resonance (NMR) spectrums analysis of isolated TAN-1612 from A. niger (FIG. 41B).

FIGS. 42A-42B provide the chemical structures of tetracycline and its analogs (FIG. 42A), and the toxicity of TAN-1612 in S. cerevisiae cultured in complete synthetic medium (CSM) (FIG. 42B).

FIG. 43 provides the toxicity of TAN-1612 in S. cerevisiae cultured in non-defined medium YPD.

FIG. 44 provides the toxicity assay of testing four different efflux pumps from A. niger in yeast strain BJ5464-NpgA.

FIG. 45 illustrates testing TAN-1612 production in the presence of four different efflux pumps from A. niger in yeast strain BJ5464-NpgA.

FIG. 46 provides TAN-1612 production in the presence of four different efflux pumps from A. niger in yeast strain BJ5464-NpgA cultured in complete synthetic medium (CSM).

FIG. 47 illustrates a biosynthetic pathway to produce TAN-1612 in S. cerevisiae.

FIG. 48 depicts testing different promoters in a biosynthetic system to produce TAN-1612 in S. cerevisiae.

FIG. 49 provides TAN-1612 productivity of BJ5464 cells culture in CSM(UT-) medium.

FIG. 50 provides TAN-1612 productivity of BJ5464 cells culture in YPD medium.

FIG. 51 illustrates building a promoter library via golden gate assembly.

FIG. 52 provides TAN-1612 productivity of top TAN-1612 yeast strains in CSM (T-) or CSM (UT-) media.

FIG. 53 provides TAN-1612 productivity of top TAN-1612 yeast strains in YPD media.

FIG. 54 provides the TAN-1612 flask production both in CSM (UT-) and YPD media.

FIG. 55 provides TAN-1612 titers quantified by supercritical fluid chromatography mass spectrometry (SFC-MS).

FIG. 56 provides the TAN-1612 flask production both in CSM (UT-) media.

FIGS. 57A-57B provide the characterization of purified TAN-1612 by NMR. FIG. 57A provides the structure of TAN-1612 and FIG. 57B show the NMR spectra.

FIG. 58 illustrates metabolic engineering to increase the titer of TAN-1612. Purple: Relevant enzymes involved in precursor production. Blue: The four biosynthetic enzymes to TAN-1612. Red: NpgA, the enzyme responsible for equipping the NRPKS AdaA with a phosphopantetheine prosthetic group. An arrow up indicates that an overexpression of the enzyme is attempted in the library of approaches for enhancing TAN-1612 biosynthetic yields in S. cerevisiae.

FIG. 59 provides differentiation of supernatants of TAN-1612 producer strains expressing or lacking an efflux pump by the FP assay. v1.0 TAN-1612 producer was developed by Tang and coworkers. Higher TAN-1612 concentrations are indicated by lower mFP units.

FIGS. 60A-60B. FIG. 60A depicts structures of tetracycline, doxycycline and minocycline differing in only three or less functional groups. FIG. 60B depicts structures of TAN-1612 and anhydrotetracycline (Atc), differing in 5 function groups. Atc and its analogue 6-demethylAtc are precursors to all FDA approved Tc derivatives. The fungal polyketide scaffold TAN-1612 is used to generate unique tetracycline analogs to be tested. Marked in grey are positions that cannot be functionalized in the α-orientation with a heteroatom functional group with previous approaches but can be with the approach disclosed herein. The stereochemistry of TAN-1612 has not yet been verified by X-Ray crystallography.

FIG. 61 depicts how 2-carboxamido functionality is introduced to TAN-1612 analogs by employing the malonamoyl CoA starting material. Steps 1-3 furnish the 4α-dimethylamino functionality (Sub-Aim 3b) and steps 4-6 furnish the target 6α-hydroxy and glycosylate it to form the target 6-demethyl-6-epiglycotetracyclines (Sub-Aim 3c). As an alternative, steps 4-6 can also take place before step 3 or before steps 1-3 to furnish the same final products.

FIG. 62 depicts proposed synthesis for proxy analogs of tetracycline analogs shown in FIG. 61 for generating TetR mutants. Compounds 12, 13 and 16 are formed by steps 5, 6 and 7′. NCS=N-chlorosuccinimide, DMP=Dess-Martin periodinane.

FIG. 63 depicts a generic structure of tetracycline and tetracycline analogues.

FIG. 64 shows the killing activity of the K2 toxin peptide at different temperatures. K2-secreting yeast cells were spotted onto a lawn of sensitive S. cerevisiae cells and the plates were incubated at the indicated temperatures. Killing zones were measured after 48 hours of incubation and are given as killing activity on the Y-axis.

FIGS. 65A-65J provide the results of halo assays performed to monitor the growth inhibition of potentially susceptible fungal strains in the presence of a yeast strain genetically engineered to express the peptide toxin K2 or K28 at high levels as compared to a parental strain. FIG. 65A shows a lawn of Saccharomyces boulardii and K28-secreting S. cerevisiae in the middle. FIG. 65B shows a lawn of Saccharomyces boulardii and K2-secreting S. cerevisiae in the middle. FIG. 65C shows a lawn of Pichia pastoris and K28-secreting S. cerevisiae in the middle. FIG. 65D shows a lawn of Pichia pastoris and K2-secreting S. cerevisiae in the middle. FIG. 65E shows a lawn of C. albicans and K28-secreting S. cerevisiae in the middle. FIG. 65F shows a lawn of C. albicans and K2-secreting S. cerevisiae in the middle. FIG. 65G shows a lawn of parent S. cerevisiae without killer toxin plasmid and K2-secreting S. cerevisiae in the middle. FIG. 65H shows a lawn of parent S. cerevisiae without killer toxin plasmid and K28-secreting S. cerevisiae in the middle. FIG. 65I shows a lawn of K28-secreting S. cerevisiae and K28-secreting S. cerevisiae in the middle. FIG. 65J shows a lawn of K2-secreting S. cerevisiae and K2-secreting S. cerevisiae in the middle.

FIGS. 66A-66C provide the results of halo assays performed to monitor the growth inhibition of Ganoderma resinaceum in the presence of a yeast strain genetically engineered to express the peptide toxin K2 or K28 at high levels. FIG. 66A shows a lawn of parent S. cerevisiae without killer toxin plasmid and Ganoderma in the middle. FIG. 66B shows a lawn of Lawn of K2-secreting S. cerevisiae and Ganoderma in the middle. FIG. 66C shows a lawn of K28-secreting S. cerevisiae and Ganoderma in the middle.

FIGS. 67A-67B provides the hypothesized functional setup in the conversion of anhydrotetracycline to tetracycline in a +OxyS+CtcM+FNO yeast cell lysate in the presence of NADPH, Fo and G6P. FIG. 67A provides the hypothesized route for the conversion of anhydrotetracycline (1) to tetracycline (3) using +OxyS+CtcM+FNO strain shown in black; 2b and 3 were isolated following incubation of a +OxyS and a +OxyS +CtcM+FNO S. cerevisiae cell lysate with 1, respectively; the conversion of 5(5a)-dehydrotetracycline (2b) to oxytetracycline (5) shown in gray was expected when a +OxyS +OxyR+FNO cell lysate was used but was not observed. FIG. 67B provides the hypothesized redox cascade to furnish FoH2 for the reduction step of 2a to 3 by CtcM in yeast cell lysate supplied with Fo, NADPH and G6P, also expressing the enzymes FNO heterologously and G6PD natively. Gray squares emphasize the carbons at which key chemical transformations occur. Fo—7,8-didemethyl-8-hydroxy-5-deazariboflavin; FNO—F420-NADP oxidoreductase from A. fulgidus; G6P—glucose-6-phosphate; 6PGL—6-phosphogluconate; G6PD—glucose-6-phosphate dehydrogenase.

FIG. 68 provides a schematic showing the key interactions of 1H-1H COSY and HMBC in the NMR of 5(5a)-dehydrotetracycline (2b) and tetracycline purified from cell lysate reaction of +OxyS and +OxyS+CtcM+FNO strains, respectively (methanol-d4, 500 MHz).

FIGS. 69A-69D provide the mass spectrometry analysis of anhydrotetracycline hydroxylation and reduction in lysates of S. cerevisiae cells expressing OxyS, CtcM and FNO in the absence of Fo and G6P (FIG. 69A), in the presence of Fo (FIG. 69B), in the presence of G6P (FIG. 69C) and in the presence of Fo and G6P (FIG. 69D). Cell lysates of +OxyS+CtcM+FNO (A. fulgidus) strain EH-6-77-3 (______) or the +OxyS −CtcM −FNO control strain EH-3-204-9 ( [symbol] ) were placed overnight in Tris buffer (100.0 mM, pH 7.45) containing anhydrotetracycline (5.4 mM), glucose (27.8 mM), NADPH (3.0 mM), Fo (0.4 mM (+FO) or 0 mM (−FO)), glucose-6-phosphate (10.0 mM (+G6P) or 0 mM (−G6P)) and mercaptoethanol (18.5 mM). m/z calculations for protonated anhydrotetracycline (1, C₂₂H₂₃N₂O₇+), 427.15; found, 427.0-427.2 [M+H]+. m/z calculations for protonated 5a(11a)-dehydrotetracycline or 5(5a)-dehydrotetracycline (2a and 2b, respectively, C₂₂H₂₃N₂O₈+), 443.15; found, 443.1-443.2 [M+H]+. m/z calculations for protonated tetracycline (3, C₂₂H₂₅N₂O₈+), 445.16; found, 445.1-445.2 [M+H]+. The expected [M+H]+value for 2 holds for either 2a or 2b as the two are isomers (Scheme 1). Fo—7,8-didemethyl-8-hydroxy-5-deazariboflavin; FNO—F420-NADP oxidoreductase from A. fulgidus; G6P—glucose-6-phosphate.

FIG. 70 provides an OxyS crystal structure (purple, 10.1021/ja403516u, PDB: 4K2X) aligned with a homologous hydroxylase crystal structure which has a tetracyclic ligand bound (rainbow, 10.1016/j.jmb.2009.09.003, PDB: 3IHG).

FIG. 71 provides UV/Vis chromatograms of the HPLC separation of extracts from +/−PgaE strains and schematics showing PgaE catalyzing its natural substrate, and the hypothesized reaction of PgaE catalyzing TAN-1612. In the excitation and emission spectra, the positive control had a lower absorbance compared to the negative control.

FIG. 72 provides a mass spectrometry analysis that shows one mass at 445.0776 m/z that appeared in the positive control (PgaE) and not in the negative control (empty plasmid). Yeast cultures of OxyS L44F, G45A, and Q299L show the most intense peaks that correspond with this mass.

FIGS. 73A-73B. FIG. 73A provides a HPLC chromatogram of OxyS Q299L. Fractions at 24, 28, 29, and 37 minutes were collected and analyzed with mass spectrometry. FIG. 73B provides a mass spectrometry analysis of OxyS Q299L purified collected fractions compared to unpurified yeast culture (from FIG. 72). No fraction corresponded to either the 2.66 or 2.74 minute elution time that would indicate the doubly hydroxylated molecule.

FIGS. 74A-74B. FIG. 74A provides an HPLC chromatogram of OxyS L44F. Fractions at 28-30, 33, 34 and 37 minutes were collected and analyzed with mass spectrometry. FIG. 74B provides a mass spectrometry analysis of OxyS L44F purified collected fractions compared to unpurified yeast culture (from FIG. 72). The purified fraction at 34 minutes corresponds to the 2.74 minute elution time, which is the hypothesized doubly hydroxylated TAN-1612.

FIGS. 75A-75B. FIG. 75A provides an HPLC chromatogram of OxyS G45A. Fractions at 25, 29, 30, 35 and 38 minutes were collected and analyzed with mass spectrometry. FIG. 75B a mass spectrometry analysis of OxyS G45A purified collected fractions compared to unpurified yeast culture (from FIG. 72). The purified fraction at 35 minutes corresponds to the 2.74 minute elution time, which is the hypothesized doubly hydroxylated TAN-1612.

FIGS. 76A-76B provide UV/Vis spectroscopy analysis of the reaction of TAN-1612 in whole cells expressing PgaE or mutant forms of OxyS (FIG. 76A). FIG. 76B is a repeat of the experiment shown in FIG. 76A.

FIG. 77 shows mass chromatograms of supernatants of unlysed S. cerevisiae cells expressing OxyS incubated with anhydrotetracycline. Pelleted unlysed cells of strain EH-3-98-6 expressing OxyS (FIG. 77A) or pelleted control cells of strain EH-3-80-3 expressing no hydroxylase (FIG. 77B) were redissolved in H₂O and added as the last component to culture tubes containing anhydrotetracycline HCl, glucose and Tris buffer (pH 7.45). The culture tubes were placed in a shaker at 21° C. for 27 h at 350 rpm. Concentrations of anhydrotetracycline HCl, glucose and Tris were 7.5 mM, 111.0 mM and 100.0 mM, respectively. Each chromatogram shows the ion percent by time for ions of m/z values within the range on the right side of each chromatogram. Thus, for row 1, 2, 3, 4, 5 and 6 the range of ions counted is total ion count (TIC), 461.156±0.03 Da (the expected mass for oxytetracycline, 5), 459.140±0.03 Da (the expected mass for 5a(11a)-dehydrooxytetracycline, 4), 445.161±0.03 Da (the expected mass for tetracycline, 3), 443.145±0.03 Da (the expected mass for dehydrotetracycline, 2) and 427.150±0.03 (the expected mass for anhydrotetracycline, 1), respectively.

FIGS. 78A-78B provide the Proton-Deuterium exchange supports 5(5a)-dehydrotetracycline and 5a(11a)-dehydrotetracycline interconversion. FIG. 78A provides proton peak at 5.67 ppm assigned to 5-H of 5(5a)-dehydrotetracycline disappears as a function of time in methanol-d4 at 27° C. FIG. 78B provides the hypothesized mechanism for 5(5a)-dehydrotetracycline and 5a(11a)-dehydrotetracycline interconversion leading to deuterium-proton exchange at the 5^{th} position of 5(5a)-dehydrotetracycline and the resulting 5.67 peak extinguishment.

FIGS. 79A-79D provide mass chromatograms of supernatants of engineered unlysed S. cerevisiae cells expressing OxyS, CtcM and FNO or control cells expressing OxyS, incubated with anhydrotetracycline and Fo. Pelleted unlysed cells of strain EH-6-77-3 expressing OxyS, CtcM and FNO (FIGS. 79A-79B) or pelleted control cells of strain EH-3-204-9 expressing OxyS and not expressing CtcM and FNO (FIGS. 79C-79D) were redissolved in H₂O and added as the last component to culture tubes containing anhydrotetracycline HCl, glucose, Tris buffer (pH 7.45) and, in the case of a and c, Fo. The culture tubes were placed in a shaker at 21° C. for 27 h at 350 rpm. Concentrations of anhydrotetracycline HCl, glucose and Tris were 7.5 mM, 111.0 mM and 100.0 mM, respectively. The concentration of Fo was 0.4 mM in a and c, and 0 mM in b and d. Each chromatogram shows the ion percent by time for ions of m/z values within the range on the right side of each chromatogram. Thus, for row 1, 2, 3 and 4 the range of ions counted is total ion count (TIC), 445.161±0.03 Da (the expected mass for tetracycline, 3), 443.145±0.03 Da (the expected mass for dehydrotetracycline, 2) and 427.150±0.03 (the expected mass for anhydrotetracycline, 1), respectively.

FIG. 80 provides the 1H-NMR spectrum of 5(5a)-dehydrotetracycline (methanol-d4, 500 MHz) purified from reaction of cell lysate of EH-3-248-1 expressing OxyS with anhydrotetracycline.

FIG. 81 provides the COSY spectrum of 5(5a)-dehydrotetracycline (2b, methanol-d4, 500 MHz) purified from reaction of cell lysate of EH-3-248-1 expressing OxyS with anhydrotetracycline.

FIG. 82 provides the HMBC spectrum (coupling constant=5 Hz) of 5(5a)-dehydrotetracycline (2b, methanol-d4, 500 MHz) purified from reaction of cell lysate of EH-3-248-1 expressing OxyS with anhydrotetracycline.

FIG. 83 provides the HMBC spectrum (coupling constant=10 Hz) of 5(5a)-dehydrotetracycline (2b, methanol-d4, 500 MHz) purified from reaction of cell lysate of EH-3-248-1 expressing OxyS with anhydrotetracycline.

FIG. 84 provides the HSQC spectrum of 5(5a)-dehydrotetracycline (2b, methanol-d4, 500 MHz) purified from reaction of cell lysate of EH-3-248-1 expressing OxyS with anhydrotetracycline.

FIG. 85 provides the 13C-NMR spectrum of 5(5a)-dehydrotetracycline (2b, methanol-d4, 500 MHz) purified from reaction of cell lysate of EH-3-248-1 expressing OxyS with anhydrotetracycline.

FIG. 86 provides the mass spectrum of 5(5a)-dehydrotetracycline (2b) purified from reaction of cell lysate of EH-3-248-1 expressing OxyS with anhydrotetracycline in ES+ and ES-ionization.

FIG. 87 provides the NMR spectrum of tetracycline standard (top) and tetracycline purified from cell lysate reaction of +OxyS+CtcM+FNO (A. fulgidus) strain with anhydrotetracycline (bottom). Tetracycline standard is tetracycline HCl, dissolved in 99.9% H2O/0.1% TFA and MeCN and dried.

FIG. 88 provides the LCMS and HRMS of tetracycline standard (top) and tetracycline purified from reaction of cell lysate of EH-6-77-3 expressing OxyS, CtcM and FNO with anhydrotetracycline (bottom) in ES+ionization. Tetracycline standard is tetracycline HCl, dissolved in 99.9% H2O/0.1% TFA and MeCN and dried.

FIG. 89 provides the structures of anhydrotetracycline, 5a(11a)-dehydrotetracycline, 5(5a)-dehydrotetracycline and tetracycline along with the corresponding structures associated with oxytetracycline and chlortetracycline. Previously isolated and characterized compound are colored blue, compounds that were not previously isolated and characterized are colored gray, 5(5a)-dehydrotetracycline (2b) that was hypothetical prior to this study and was isolated and characterized for the first time in this study is colored green.

### DETAILED DESCRIPTION

The present disclosure provides genetically-engineered fungal cells, where the genetically-engineered fungal cells autonomously generate and/or secrete therapeutic molecules for treating a person in need of such therapeutic molecules. In certain embodiments, the genetically-engineered fungal cells can autonomously generate and/or secrete such therapeutic molecules by an engineered biosynthesis pathway.

For clarity, but not by way of limitation, the detailed description of the presently disclosed subject matter is divided into the following subsections:

Definitions;

II. Therapeutic Molecules;

III. Genetically-Engineered Cells;

IV. Methods of Use; and

V. Pharmaceutical Compositions.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. In certain embodiments, the polypeptide can be endogenous to the cell, or preferably, can be exogenous, meaning that they are heterologous, i.e., foreign, to the cell being utilized, such as a synthetic peptide produced by a yeast cell. In certain embodiments, synthetic peptides are used, more preferably those which are directly secreted into the medium.

The term “protein” as used herein refers to a sequence of amino acids for which the chain length is sufficient to produce the higher levels of tertiary and/or quaternary structure. This is to distinguish from “peptides” that typically do not have such structure.

Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

The term “functional fragment thereof,” as used herein, refers to a fragment of a therapeutic molecule, e.g., a protein or peptide, that retains at least a portion of the activity of the intact and/or full-length therapeutic molecule, e.g., a protein or peptide. In certain embodiments, the functional fragment retains at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the activity of the intact and/or full-length therapeutic molecule.

As used herein the terms “amino acid,” “amino acid monomer” or “amino acid residue” refer to organic compounds composed of amine and carboxylic acid functional groups, along with a side-chain specific to each amino acid. In particular, alpha- or α-amino acid refers to organic compounds in which the amine (—NH₂) is separated from the carboxylic acid (—COOH) by a methylene group (—CH₂), and a side-chain specific to each amino acid connected to this methylene group (—CH₂) which is alpha to the carboxylic acid (—COOH). Different amino acids have different side chains and have distinctive characteristics, such as charge, polarity, aromaticity, reduction potential, hydrophobicity and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a nucleic acid of the disclosure in vitro and/or in vivo, e.g., in a yeast cell. For example, but not by way of limitation, a nucleic acid of the present disclosure can encode NpgA, AdaA, AdaB, AdaC, AdaD or any efflux pump. Such DNA (e.g., cDNA) or RNA (e.g., mRNA) vectors can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., a therapeutic.

As used herein, the term “recombinant protein” refers generally to peptides and proteins. Such recombinant proteins are “heterologous,” i.e., foreign to the cell being utilized, such as a heterologous secretory peptide produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, the term “fusion protein” refers to a protein that includes all or a portion of a protein that is linked, e.g., at the N-terminus or C-terminus, to a second protein or a portion of the second protein.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing fusion protein genetic molecular components with codon ensuring optimized expression in assay systems as will be understood by a skilled person.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide. In certain embodiments, secretion can utilize the classical secretory pathway of yeast. In certain embodiments, secretion can utilize an efflux pump.

The term “binding,” as used herein, refers to the connecting or uniting of two or more components by a interaction, bond, link, force or tie in order to keep two or more components together, which encompasses either direct or indirect binding where, for example, a first component is directly bound to a second component, or one or more intermediate molecules are disposed between the first component and the second component. Exemplary bonds comprise covalent bond, ionic bond, van der Waals interactions and other bonds identifiable by a skilled person. In certain embodiments, the binding can be direct, such as the production of a polypeptide scaffold that directly binds to a scaffold-binding element of a protein. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component. Examples described herein include, without limitation, binding of tetracyclines or its analogs to the 30S ribosomal subunits. As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “derived” or “derive” is used herein to mean to obtain from a specified source.

The term “molecule,” as used herein, refers a group of atoms bonded together, representing the smallest fundamental unit of a chemical compound that can take part in a chemical reaction.

As used herein, the term “therapeutic molecule” includes any small molecule and peptide that can be administered to a subject and provide a therapeutic effect, such as reduce, alleviate, or eliminate symptoms or pathologies of a disease or disorder.

“Pharmaceutically acceptable carrier,” as used herein, refers to a pharmaceutically acceptable material, composition, or vehicle that is involved in carrying or transporting a compound or composition of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

As used herein the term “subject” refers to any animal (e.g., a mammal), including, but not limited to, humans, non-human primates, rodents, and the like, which is to be recipient of a particular treatment.

### II. Molecules

The present disclosure provides cells that express and/or secrete one or more molecules, e.g., therapeutic molecules. For example, but not by way of limitation, a cell, e.g., a genetically-engineered cell, of the present disclosure can produce and/or secrete one molecule. In certain embodiments, a cell, e.g., a genetically-engineered cell, of the present disclosure can produce and/or secrete more than one molecule, e.g., two molecules, three molecules, four molecules or five molecules or more.

In certain embodiments, a multi-cell system can be used for the generation of pharmaceuticals that require the assembly of multiple components in a coordinated manner, where each cell is configured to produce a component of a pharmaceutical. In certain embodiments, a multi-cell system can be used for the generation of multiple different molecules. In certain embodiments, a multi-cell system can be used for the generation of 2, 3, 4, 5, 6, 7, 8, 9 or 10 different molecules. Non-limiting examples of such multi-cell systems are disclosed PCT/US2020/030795, the contents of which is incorporated herein in its entirety.

In certain embodiments, the molecule can be a small molecule, e.g., a small therapeutic molecule. In certain embodiments, the molecule can be a peptide, e.g., a therapeutic peptide or functional fragment thereof. In certain embodiments, a genetically engineered cell, e.g., genetically engineered fungal cell, of the present disclosure expresses a small therapeutic molecule. In certain embodiments, a genetically-engineered cell can express one or more small molecule therapeutics and/or one or more peptide therapeutics.

In certain embodiments, the molecule, e.g., therapeutic molecule, expressed by a genetically-engineered cell of the present disclosure is secretable. For example, but not by way of limitation, the molecule, e.g., therapeutic molecule, can be expressed intracellularly in a cell and subsequently transported to the plasma membrane of the cell and secreted to the exterior of the cell, e.g., outside the plasma membrane of the cell. In certain embodiments, the molecule, e.g., therapeutic molecule, can be secreted using the mating secretory pathway. In certain embodiments, the molecule, e.g., therapeutic molecule, can be secreted using as efflux pump, as described herein.

In certain embodiments, secretion can be performed using the conserved secretory pathway in fungal cells, e.g., yeast. For example, but not by way of limitation, a molecule is secretable because it is coupled to a secretion signal sequence. Examples of secretion signal sequences can be obtained from proteins including mating factor alpha-1, alpha factor K, alpha factor T, glycoamylase, inulinase, invertase, lysozyme, serum albumin, alpha-amylase and killer protein. In certain embodiments, the secretion signal sequence is a secretion signal sequence obtained from a yeast protein, such as a Saccharomyces cerevisiae protein. In certain embodiments, the secretion signal peptide is obtained from Saccharomyces cerevisiae mating factor alpha-1. Additionally, mutations, substitutions and truncations of any signal peptide are also within the scope of the present disclosure. The selection and design, including additional mutations and truncations of a signal peptide is within the ability and discretion of one of ordinary skill in the art. In certain embodiments, the one or more secretion signal sequences are located at the N-terminus of a secretable peptide. In certain embodiments, a Kex2 processing site and/or a Ste13 processing site or a homolog thereof can be present between the amino acid sequence of the secretion signal sequence and the secretable peptide. Additional non-limiting examples of secretion signals are disclosed in U.S. Pat. No. 10,725,036, the contents of which is disclosed herein in its entirety.

Small Therapeutic Molecules

In certain embodiments, the molecule, e.g., therapeutic molecule, can be a small therapeutic molecule. In certain embodiments, genetically-engineered or non-genetically engineered cells, e.g., modified strain of yeasts, that produce and/or secrete a small therapeutic molecule by engineered biosynthesis. Small therapeutic molecules are molecules with a low molecular weight, generally less than about 900 Daltons.

In certain embodiments, the small molecule therapeutic is one or more of an antibiotic, an anti-inflammatory, an antifungal or an antimicrobial small molecule. In certain embodiments, the small molecule therapeutic is an antibiotic. In certain embodiments, the small molecule therapeutic is an anti-inflammatory. In certain embodiments, the small molecule therapeutic is an antifungal. In certain embodiments, the small molecule therapeutic is an antimicrobial.

In certain embodiments, the small molecule therapeutic has one or more of the following properties: anti-inflammatory properties, antibiotic properties, antimicrobial properties and/or antifungal properties. In certain embodiments, the small molecule therapeutic is a small molecule that has anti-inflammatory properties. In certain embodiments, the small molecule therapeutic is a small molecule that has antibiotic properties. In certain embodiments, the small molecule therapeutic is a small molecule that has antifungal properties. In certain embodiments, the small molecule therapeutic is a small molecule that has antimicrobial properties.

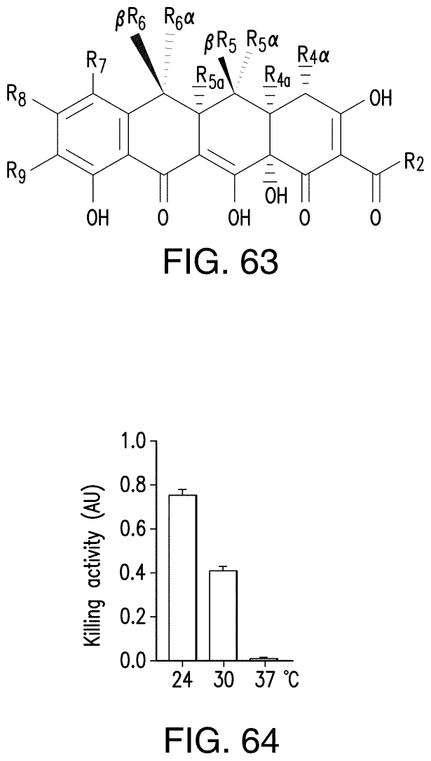

In certain embodiments, the small therapeutic molecule is tetracycline or an analogue thereof. A general structure of a tetracycline analogue is depicted in FIG. 63. Each of R₂, R_{4α}, R₄ₐ, R_{5α}, R_{5β}, R₅ₐ, R_{6α}, R_{6β}, R₇, R₈, R₉ can be a functional group including, but not limited to, H, R, NRR′ OH, OR, SR, SOR, NRCOR′, nitro, sulfonate, COMe, CONRR′ and glycoside, where R and R′ could be H, alkyl or aryl.

In certain embodiments, the tetracycline analogue can be TAN-1612. In certain embodiments, the tetracycline analogue is doxycycline. In certain embodiments, the tetracycline analogue is a 9-amido-tetracycline. In certain embodiments, the tetracycline analogue is chlortetracycline. In certain embodiments, the tetracycline analogue is oxytetracycline. In certain embodiments, the tetracycline analogue is demeclocycline. In certain embodiments, the tetracycline analogue is meclocycline. In certain embodiments, the tetracycline analogue is metacycline. In certain embodiments, the tetracycline analogue is doxycycline. In certain embodiments, the tetracycline analogue is minocycline. In certain embodiments, the tetracycline analogue is tigecycline. In certain embodiments, the tetracycline analogue is omadacycline. In certain embodiments, the tetracycline analogue is sarecycline. In certain embodiments, the tetracycline analogue is eravacycline. In certain embodiments, the tetracycline analogue is anhydrotetracycline. In certain embodiments, the tetracycline analogue is 4-de(dimethylamino)-anhydrotetracycline. In certain embodiments, the tetracycline analogue is viridicatumtoxin. In certain embodiments, the tetracycline analogue is an analogue or derivative of the above. Additional non-limiting examples of tetracycline analogues are disclosed in U.S. Pat. No. 8,486,921, the contents of which are incorporated herein in its entirety.

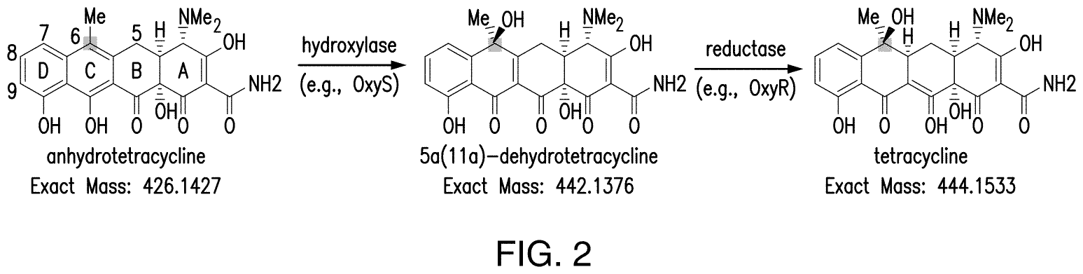

In certain embodiments, the tetracycline or TAN-1612 analogue can include one or more modifications at any one of the rings of tetracycline or TAN-1612 (see FIG. 2 for ring numbering). In certain embodiments, the tetracycline or TAN-1612 analogue can include a modification at the A ring of tetracycline or TAN-1612. In certain embodiments, the tetracycline or TAN-1612 analogue can include a modification at the B ring of tetracycline or TAN-1612. In certain embodiments, the tetracycline or TAN-1612 analogue can include a modification at the C ring of tetracycline or TAN-1612. In certain embodiments, the tetracycline or TAN-1612 analogue can include a modification at the D ring of tetracycline or TAN-1612. In certain embodiments, the tetracycline or TAN-1612 analogue can include one or more modifications at the A ring, B, ring, C ring and/or D ring. In certain embodiments, the tetracycline or TAN-1612 analogue can include one or more modifications at the A ring and C ring.

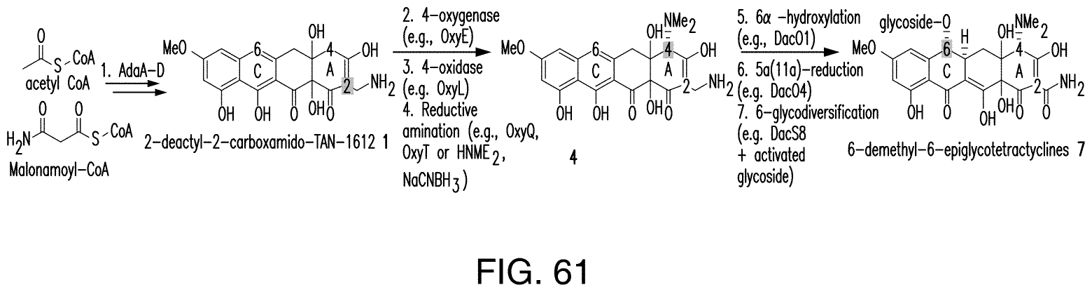

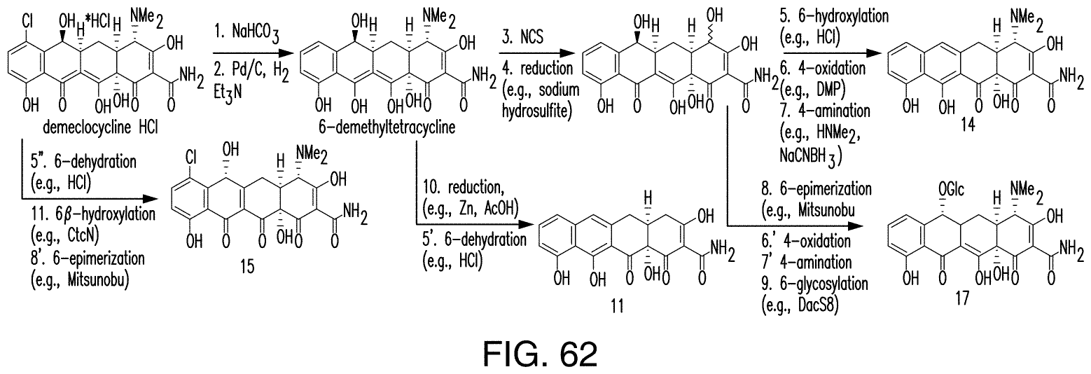

In certain embodiments, the tetracycline or TAN-1612 analogue can include a modification at any one or more carbon positions of tetracycline, TAN-1612 or an analogue thereof (see FIG. 2 for carbon numbering). In certain embodiments, tetracycline, TAN-1612 or an analogue thereof can include a modification at the 2 position. In certain embodiments, tetracycline, TAN-1612 or an analogue thereof can include a modification at the 4 position. In certain embodiments, tetracycline, TAN-1612 or an analogue thereof can include a modification at the 5 position. In certain embodiments, tetracycline, TAN-1612 or an analogue thereof can include a modification at the 6 position. In certain embodiments, tetracycline, TAN-1612 or an analogue thereof can include a modification at the 7 position. In certain embodiments, tetracycline, TAN-1612 or an analogue thereof can include a modification at the 9 position. In certain embodiments, tetracycline, TAN-1612 or an analogue thereof can include one or more, two or more, three or more, four or more, five or more or six modifications at the 2, 4, 5, 6, 7 or 9 positions. In certain embodiments, tetracycline, TAN-1612 or an analogue thereof can include modifications at the 2 and 4 positions (see FIGS. 61 and 62).

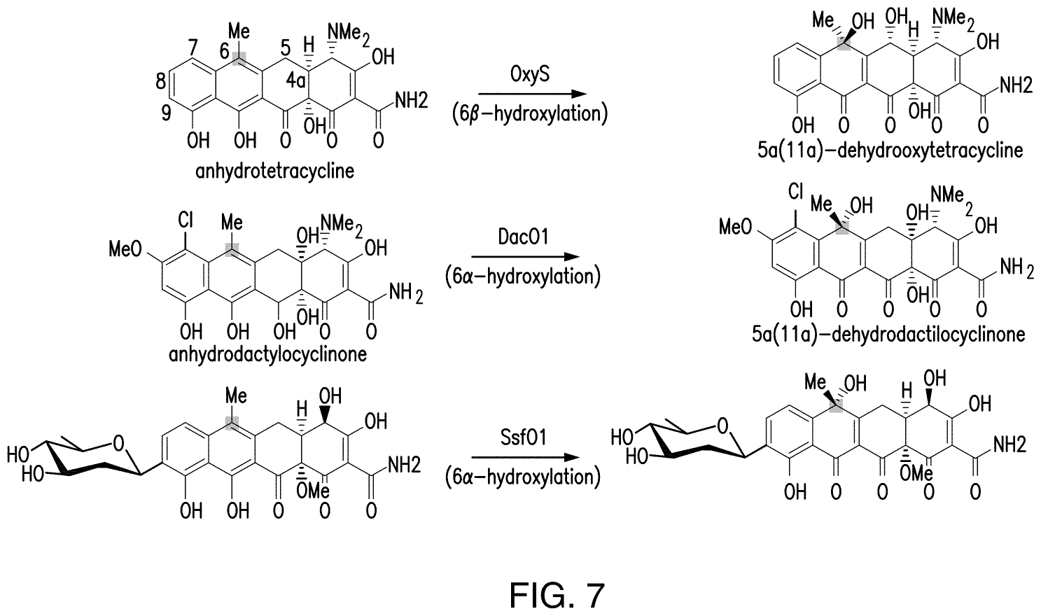

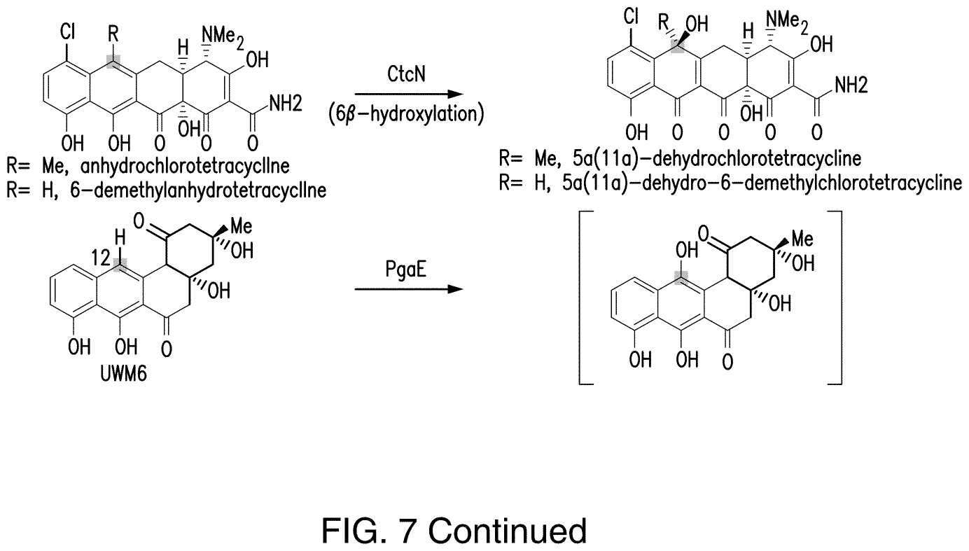

In certain embodiments, the tetracycline or TAN-1612 analogue can include the addition of one or more hydroxyl groups to tetracycline, TAN-1612 or an analogue thereof. For example, but not by way of limitation, the 5 position and/or the 6 position of tetracycline, TAN-1612 or an analogue thereof can be modified by a hydroxyl group (see FIGS. 7, 19, 20, 33, 61, 62, 67 and 71).

In certain embodiments, the tetracycline or TAN-1612 analogue can include the addition of one or more glycosyl groups to tetracycline, TAN-1612 or an analogue thereof. For example, but not by way of limitation, the 6 position of tetracycline, TAN-1612 or an analogue thereof can be modified by a glycosyl group (see FIG. 61).

In certain embodiments, the small molecule, e.g., therapeutic small molecule, is not a vitamin.

Peptide Therapeutics

In certain embodiments, the molecule, e.g., therapeutic molecule, can be a peptide. For example, but not by way of limitation, a genetically-engineered cell of the present disclosure produces and/or secretes peptides.

In certain embodiments, the molecule, e.g., therapeutic molecule, can be a peptide, e.g., therapeutic peptide. For example, but not by way of limitation, genetically engineered cells of the present disclosure produce and/or secrete peptides. In certain embodiments, the peptides, e.g., therapeutic peptides, can be composed of about 3-50 amino acid residues. In certain embodiments, the 3-50 amino acid residues can be continuous within a larger polypeptide or protein or can be a group of 3-50 residues that are discontinuous in a primary sequence of a larger polypeptide or protein but that are spatially near in three-dimensional space. In certain embodiments, the peptide can be a part of a peptide, a part of a full protein or polypeptide and can be released from that protein or polypeptide by proteolytic treatment or can remain part of the protein or polypeptide.

In certain embodiments, the peptide, e.g., therapeutic peptide, can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the GPCR peptide ligand has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the peptide a length of about 5 to about 30 residues.

In certain embodiments, the peptide has a length of 9 residues. In certain embodiments, the peptide has a length of 10 residues. In certain embodiments, the peptide has a length of 11 residues. In certain embodiments, the peptide has a length of 12 residues. In certain embodiments, the peptide has a length of 13 residues. In certain embodiments, the peptide has a length of 14 residues. In certain embodiments, the peptide has a length of 15 residues. In certain embodiments, the peptide has a length of 16 residues. In certain embodiments, the peptide has a length of 17 residues. In certain embodiments, the peptide has a length of 18 residues. In certain embodiments, the peptide has a length of 19 residues. In certain embodiments, the peptide has a length of 20 residues. In certain embodiments, the peptide has a length of 21 residues. In certain embodiments, the peptide has a length of 22 residues. In certain embodiments, the peptide has a length of 23 residues. In certain embodiments, the peptide has a length of 24 residues. In certain embodiments, the peptide has a length of 25 residues. In certain embodiments, the peptide has a length of 26 residues. In certain embodiments, the peptide has a length of 27 residues. In certain embodiments, the peptide has a length of 28 residues. In certain embodiments, the peptide has a length of 29 residues. In certain embodiments, the peptide has a length of 30 residues. In certain embodiments, the peptide has a length of 31 residues. In certain embodiments, the peptide has a length of 32 residues. In certain embodiments, the peptide has a length of 33 residues. In certain embodiments, the peptide has a length of 34 residues. In certain embodiments, the peptide has a length of 35 residues. In certain embodiments, the peptide has a length of 36 residues. In certain embodiments, the peptide has a length of 37 residues. In certain embodiments, the peptide has a length of 38 residues. In certain embodiments, the peptide has a length of 39 residues. In certain embodiments, the peptide has a length of 40 residues. In certain embodiments, the peptide has a length of 41 residues. In certain embodiments, the peptide has a length of 42 residues. In certain embodiments, the peptide has a length of 43 residues. In certain embodiments, the peptide has a length of 44 residues. In certain embodiments, the peptide has a length of 45 residues. In certain embodiments, the peptide has a length of 46 residues. In certain embodiments, the peptide has a length of 47 residues. In certain embodiments, the peptide has a length of 48 residues. In certain embodiments, the peptide has a length of 49 residues. In certain embodiments, the peptide has a length of 50 residues.

In certain embodiments, the peptide therapeutic is an antibiotic, an antifungal or an antimicrobial peptide. In certain embodiments, the peptide therapeutic is an antibiotic peptide. In certain embodiments, the peptide therapeutic is an antifungal peptide. In certain embodiments, the peptide therapeutic is an antimicrobial peptide.

In certain embodiments, the peptide therapeutic has one or more of the following properties: antibiotic properties, antimicrobial properties and/or antifungal properties. In certain embodiments, the peptide therapeutic is a peptide that has anti-inflammatory properties. In certain embodiments, the peptide therapeutic is a peptide that has antibiotic properties. In certain embodiments, the peptide therapeutic is a peptide that has antifungal properties. In certain embodiments, the peptide therapeutic is a peptide that has antimicrobial properties.

In certain embodiments, the peptide therapeutic is a fungal toxin peptide. In certain embodiments, the fungal toxin peptide has antifungal properties, antibiotic properties and/or antimicrobial properties. Non-limiting examples of such fungal toxin peptides include a K1, K2 or K28 toxin peptide. In certain embodiments, the K1, K2 or K28 toxin peptide is derived from Saccharomyces cerevisiae. In certain embodiments, the fungal toxin peptide is the K1 toxin peptide derived from Saccharomyces cerevisiae. In certain embodiments, the fungal toxin peptide is the K2 toxin peptide derived from Saccharomyces cerevisiae. In certain embodiments, the fungal toxin peptide is the K28 toxin peptide derived from Saccharomyces cerevisiae.

In certain embodiments, the fungal toxin peptide can be encoded by a nucleotide sequence disclosed in Table 28. For example, but not by way of limitation, the fungal toxin peptide can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 28.

In certain embodiments, the fungal toxin peptide comprises an amino acid sequence disclosed in in Table 28. For example, but not by way of limitation, the fungal toxin peptide comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 28.

In certain embodiments, the molecule, e.g., therapeutic molecule, is not a protein. In certain embodiments, the molecule, e.g., therapeutic molecule, is not a peptide. In certain embodiments, the protein or peptide therapeutic is not an enzyme.

### III. Genetically-Engineered Cells

The present disclosure provides cells for expressing, e.g., secreting, molecules of interest, e.g., therapeutic molecules disclosed herein. For example, but not by way of limitation, cells of the present disclosure can include a nucleic acid that encodes one or more molecules of interest. Alternatively and/or additionally, cells of the present disclosure can include one or more nucleic acids that encode proteins, e.g., enzymes, that play a role in the generation of a molecule of interest and/or an intermediate of the molecule of interest. Non-limiting examples of molecules, e.g., therapeutic molecules, that can be produced by the cells of the present disclosure are disclosed in Section II. In certain embodiments, a genetically-engineered cell can express one or more small molecule therapeutics and/or one or more peptide therapeutics.

The cells used for generating and/or secreting various molecules described herein can be, e.g., genetically engineered cells. The genetically modified cells for use in generating a molecule can be a mammalian cell, a plant cell, a bacterial cell or a fungal cell. For example, but not by way of limitation, the cell can be a mammalian cell, e.g., a genetically engineered mammalian cell. In certain embodiments, the cell can be a plant cell, e.g., a genetically engineered plant cell. In certain embodiments, the cell can be a bacterial cell, e.g., a genetically engineered bacterial cell. In certain embodiments, the cell can be a fungal cell, e.g., a genetically engineered fungal cell.

Any fungal strain can be used in the present disclosure. In certain embodiments, the fungal cell can be a species from a genus including, but not limited to, Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera and Kluyveromyces. In certain embodiments, a genetically-engineered cell of the present disclosure can be a cell of Alternaria brasicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania Africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres f teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma vixens, Tuber melanosporum, Vanderwaltozyma polyspora 1, Vanderwaltozyma polyspora 2, Verticillium alfalfae, Verticillium dahliae, Wickerhamomyces ciferrii, Yarrowia lipolytica, Zygosaccharomyces bailii, Zygosaccharomyces rouxii and combinations thereof.

In certain embodiments, the genetically engineered cell of the present disclosure is a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

In certain embodiments, the genetically-engineered cell of the present disclosure is Saccharomyces cerevisiae. In certain embodiments, the genetically-engineered cell of the present disclosure is Saccharomyces boulardii. In certain embodiments, the genetically-engineered cell of the present disclosure is not Saccharomyces boulardii.

In certain embodiments, the genetically-engineered cell of the present disclosure is a bacterial cell. Non-limiting examples of bacteria include Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, Lactobacillus gasseri and Escherichia coli. In certain embodiments, the bacteria cell is Escherichia coli.

In certain embodiments, the genetically engineered cell of the present disclosure is a mammalian cell. Non-limiting examples of mammalian cells include monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells' and myeloma cell lines such as Y0, NS0 and Sp2/0.

In certain embodiments, a cell, e.g., a fungal cell, of the present disclosure has been genetically engineered to express one or more proteins, e.g., one or more enzymes, that play a role in the synthesis of a therapeutic molecule. For example, but not by way of limitation, a cell, e.g., a fungal cell, of the present disclosure has been genetically engineered to express one or more proteins, two or more proteins, three or more proteins, four or more proteins, five or more proteins, six or more proteins, seven or more proteins, eight or more proteins or nine or more proteins that are involved in the synthesis of a molecule, e.g., a therapeutic molecule. In certain embodiments, cells for use in the present disclosure can be genetically engineered to express one or more proteins, e.g., one or more enzymes, that play a role in the synthesis of tetracycline, TAN-1612 or an analogue thereof. For example, but not by way of limitation, a cell, e.g., a fungal cell, of the present disclosure has been genetically engineered to express one or more proteins, two or more proteins, three or more proteins, four or more proteins, five or more proteins, six or more proteins, seven or more proteins, eight or more proteins or nine or more proteins that are involved in the synthesis of tetracycline, TAN-1612 or an analogue thereof. In certain embodiments, a cell, e.g., a fungal cell, of the present disclosure has been genetically engineered to express one or more enzymes, two or more enzymes, three or more enzymes, four or more enzymes, five or more enzymes, six or more enzymes, seven or more enzymes, eight or more enzymes or nine or more enzymes that are involved in the synthesis of tetracycline, TAN-1612 or an analogue thereof.

In certain embodiments, the one or more proteins, e.g., one or more enzymes, that play a role in the synthesis of a molecule, e.g., a therapeutic molecule, e.g., a small molecule therapeutic, are derived from a bacterium. For example, but not by way of limitation, the one or more proteins, e.g., one or more enzymes, are derived from Thermobifida fusca, Chlamydomonas reinhardtii, Streptomyces rimosus, Mycobacterium tuberculosis, Archeoglobus fulgidus and/or Streptomyces griseus. In certain embodiments, the one or more proteins, e.g., one or more enzymes, that play a role in the synthesis of a tetracycline, TAN-1612 or an analogue thereof are derived from Thermobifida fusca, Chlamydomonas reinhardtii, Streptomyces rimosus, Mycobacterium tuberculosis, Archeoglobus fulgidus and/or Streptomyces griseus.

In certain embodiments, the one or more proteins, e.g., one or more enzymes, that play a role in the synthesis of a molecule, e.g., a therapeutic molecule, e.g., a small molecule therapeutic, are derived from a fungus. For example, but not by way of limitation, the one or more proteins, e.g., one or more enzymes, are derived from Aspergillus nidulans and/or Aspergillus niger. In certain embodiments, the one or more proteins, e.g., one or more enzymes, that play a role in the synthesis of tetracycline, TAN-1612 or an analogue thereof are derived from Aspergillus nidulans and/or Aspergillus niger.

In certain embodiments, a cell of the present disclosure is genetically engineered to express one or more proteins, e.g., one or more enzymes, that play a role in the synthesis of a molecule, e.g., a therapeutic molecule. Non-limiting examples of such enzymes include transferases, synthases, lactamases, monooxygenases, reductases, hydroxylases, oxidoreductases and glycotransferases. In certain embodiments, a cell disclosed herein can be genetically modified to express one or more, two or more, three or more, four or more, five or more, six or more, seven or more, nine or more or ten or more enzymes selected from transferases, synthases, lactamases, monooxygenases, reductases, hydroxylases, oxidoreductases and glycotransferases.

In certain embodiments, the enzyme is a transferase. In certain embodiments, the transferase is a phosphopantetheinyl transferase (PPTase). Non-limiting examples of PPTases are provided in Beld et al., Natural Products Reports 31:61-108 (2014), which is incorporated herein in its entirety. In certain embodiments, the PPTase is NpgA, e.g., derived from Aspergillus nidulans. In certain embodiments, NpgA is encoded by a nucleotide sequence disclosed in Table 1.1. In certain embodiments, NpgA is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

In certain embodiments, the transferase is an O-methyltransferase (O-MT). Non-limiting examples of O-methyltransferases are provided in Ayabe et al., Comprehensive Natural Products II 1:929-976 (2010), which is incorporated herein in its entirety. In certain embodiments, the O-MT is AdaD, e.g., derived from Aspergillus niger. In certain embodiments, AdaD is encoded by a nucleotide sequence disclosed in Table 1.1. In certain embodiments, AdaD is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

In certain embodiments, the enzyme is a synthase. In certain embodiments, the synthase is a nonreducing polyketide synthase (NRPKS). Non-limiting examples of nonreducing polyketide synthases are provided in Schmitt et al., Phytochemistry 66(11):1241-1253 (2005), which is incorporated herein in its entirety. In certain embodiments, the NRPKS is AdaA, e.g., derived from Aspergillus niger. In certain embodiments, AdaA is encoded by a nucleotide sequence disclosed in Table 1.1. In certain embodiments, AdaA is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

In certain embodiments, the enzyme is a lactamase. In certain embodiments, the lactamase is a metallo-β-lactamase (MBL). Non-limiting examples of metallo-β-lactamases are provided in Mojica et al., Current Drug Targets 17(9):1029-1050 (2016), which is incorporated herein in its entirety. In certain embodiments, the MBL is AdaB, e.g., derived from Aspergillus niger. In certain embodiments, AdaB is encoded by a nucleotide sequence disclosed in Table 1.1. In certain embodiments, AdaB is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

In certain embodiments, the enzyme is a monooxygenase. In certain embodiments, the monooxygenase is a flavin adenine dinucleotide (FAD)-dependent monooxygenase (FMO). Non-limiting examples of FAD-dependent monooxygenases are provided in Berkel et al., Journal of Biotechnology 124(5):670-689 (2006), which is incorporated herein in its entirety. In certain embodiments, the FMO is AdaC, e.g., derived from Aspergillus niger. In certain embodiments, AdaC is encoded by a nucleotide sequence disclosed in Table 1.1. In certain embodiments, AdaC is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

In certain embodiments, the enzyme is a reductase. In certain embodiments, the enzyme is a dehydrotetracycline reductase. In certain embodiments, a reductase can be used to reduce the 5a(11a) double bond of 5a(11a)-dehydrotetracycline. Non-limiting examples of reductases include OxyR, an Fo reductase, an F420 reductase, FNO, OYE1, OYE2, OYE3, DacO4, CtcM and mutants thereof. In certain embodiments, the reductase is OxyR, e.g., derived from Streptomyces rimosus. In certain embodiments, the reductase is an F420 reductase, e.g., derived from Mycobacterium tuberculosis, Archeoglobus fulgidus or Streptomyces griseus. In certain embodiments, the F420 reductase is F420 NADPH oxidoreductase (FNO), e.g., derived from Archaeoglobus fulgidus. In certain embodiments, the reductase is CtcM, e.g., derived from Archeoglobus fulgidus. In certain embodiments, the reductase is encoded by a nucleotide sequence disclosed in Tables 2, 5 and 15. In certain embodiments, the reductase is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Tables 2, 5 and 15.

In certain embodiments, the enzyme is a hydroxylase. Non-limiting examples of hydroxylases include PgaE, OxyS, SsfO1, CtcN, DacO1 and mutants thereof. In certain embodiments, the enzyme is an anhydrotetracycline hydroxylase. In certain embodiments, the hydroxylase is OxyS, e.g., derived from Streptomyces rimosus, or a mutant thereof. In certain embodiments, the hydroxylase is encoded by a nucleotide sequence disclosed in Tables 2, 5, 15, 17 and 36-39. In certain embodiments, the hydroxylase is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Tables 2, 5, 15, 17 and 36-39. In certain embodiments, the hydroxylase comprises an amino acid sequence disclosed in Table 40.

In certain embodiments, the hydroxylase comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 40.

Additional non-limiting examples of enzymes that can be expressed in a genetically-engineered cell to synthesize a therapeutic molecule of the present disclosure include DacA, DacB, DacC, DacD, DacG, DacH, DacK, DacM1, DacM2, DacM3, DacN, DacO2, DacO1, DacO3, DacO5, DacJ, DacP, DacQ, DacE, DacT1, DacT2, DacT3, DacR1, DacR2, DacR3, DacS1, DacS2, DacS3, DacS4, DacS5, DacS6, DacS7, DacS8, DacS9, DacP1, DacP2, DacP3, OxyA, OxyB, OxyC, OxyD, OxyG, OxyF, OxyH, OxyI, OxyK, OxyL, OxyM4, OxyN, OxyP, OxyQ, TA1, OtcG, OtrA, OtrB, Ctc9, Ctc8, Ctc7, Ctc6, Ctc5, Ctc4, Ctc3, CtcA, CtcB, CtcC, CtcD, CtcE, CtcF, CtcG, CtcH, CtcI, CtcJ, CtcK, CtcL, CtcN, CtcO, CtcP, CtcQ, CtcR, CtcS, CtcT, CtcU, CtcV, CtcW, CtcX, CtcY, CtcZ, Ctc1, Ctc2, Ctc10, VrtA, VrtB, VrtC, VrtD, VrtE, VrtF, VrtG, VrtG, VrtI, VrtJ, VrtK, VrtL, VrtR1, VrtR2, SsfA, SsfB, SsfC, SsfD, SsfY1, SsfY2, SsfY4, SsfL2, SsfM4, SsfO1, SsfO2, SsfV, SsfT1, SsfT2, SsfR, SsfS1 and SsfS3. In certain embodiments, DacO1, CtcN, SsfO1, DacJ and/or DacM2 are encoded by nucleotide sequences disclosed in Table 15. In certain embodiments, DacO1, CtcN, SsfO1, DacJ and/or DacM2 are encoded by nucleotide sequences that are at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 15.

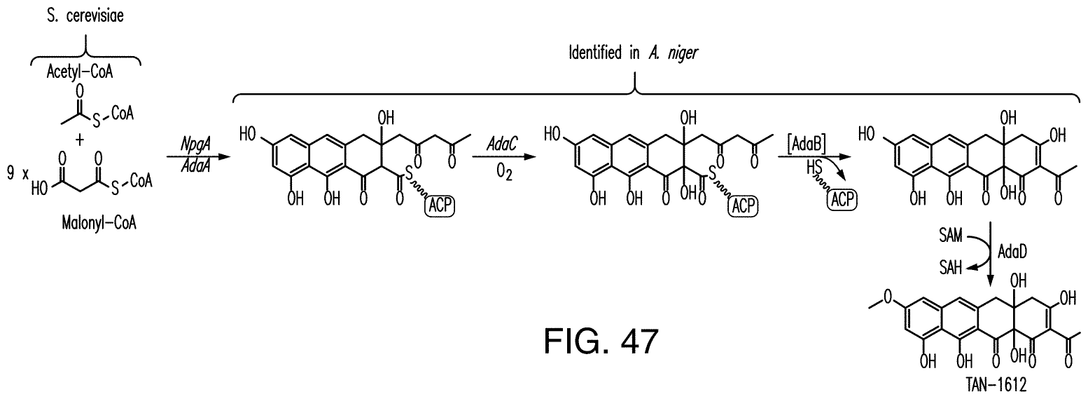

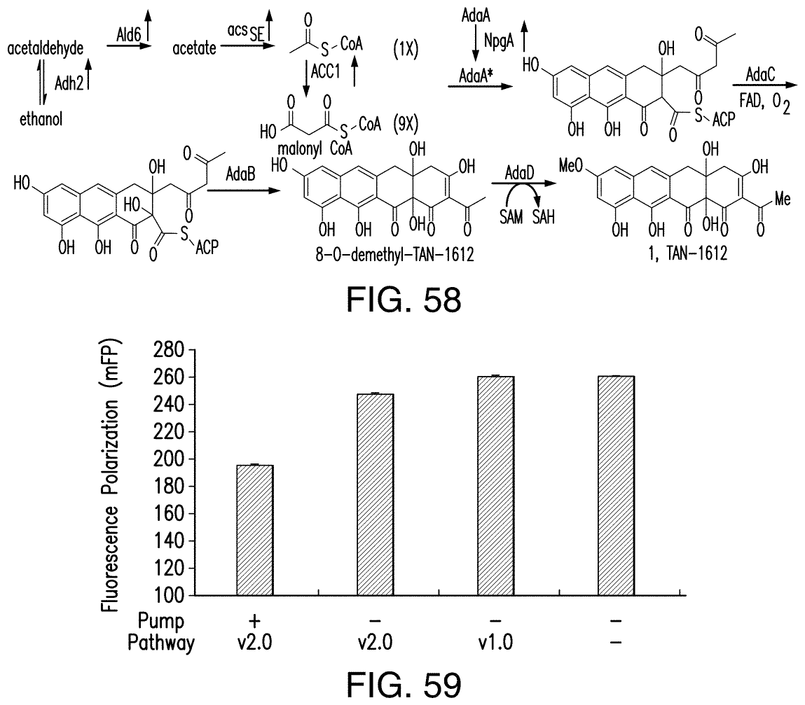

In certain embodiments, the cells for use in the present disclosure can be genetically engineered to express one or more proteins, e.g., enzymes, that can be used to synthesize the tetracycline analogue TAN-1612 or an analogue thereof. As shown in FIGS. 47 and 58, the enzymes NpgA, AdaA, AdaB, AdaC and AdaD are involved in the biosynthetic pathway of TAN-1612 from precursors acetyl-CoA and malonyl-CoA. Acetyl-CoA and malonyl-CoA are generated by the genetically-engineered cells using carbohydrate and lipid metabolic pathways, e.g., the native glycolysis pathway and the TCA cycle. In certain embodiments, the cells for use in the present disclosure can be genetically engineered to express NpgA, AdaA, AdaB, AdaC, AdaD or a combination thereof. For example, one or more of NpgA, AdaA, AdaB, AdaC and AdaD can be expressed in a genetically-engineered cell of the present disclosure to synthesize TAN-1612 or analogues thereof. In certain embodiments, one or more, two or more, three or more, four or more or all five of NpgA, AdaA, AdaB, AdaC and AdaD are expressed in a genetically-engineered disclosed herein. For example, but not by way of limitation, all four of AdaA, AdaB, AdaC and AdaD can be expressed in a genetically-engineered cell. In certain embodiments, all five of NpgA, AdaA, AdaB, AdaC and AdaD can be expressed in a genetically-engineered cell.

In certain embodiments, a genetically-engineered cell of the present disclosure can include one or more enzymes involved in the chemical modification of TAN-1612 to synthesize an analogue of TAN-1612 or to synthesize tetracycline or an analogue thereof. In certain embodiments, one or more enzymes that modify the A ring, B ring, C ring and/or D ring of TAN-1612 can be expressed in a cell of the present disclosure to synthesize a TAN-1612 analogue (see FIGS. 2 and 61). In certain embodiments, one or more enzymes that modify the A ring and/or C ring of TAN-1612 can be expressed in a cell of the present disclosure to synthesize a TAN-1612 analogue.

In certain embodiments, a genetically-engineered cell of the present disclosure can include one or more enzymes involved in the chemical modification of TAN-1612 to synthesize an analogue of TAN-1612 or to synthesize tetracycline or an analogue thereof that includes a glycosyl group. For example, but not by way of limitation, a genetically-engineered cell of the present disclosure can express a glycosyltransferase. In certain embodiments, the glycosyltransferase can be DacS8. In certain embodiment, the glycosyltransferase adds a glycosyl group to the 6 position, e.g., the 6a position (see FIG. 61).

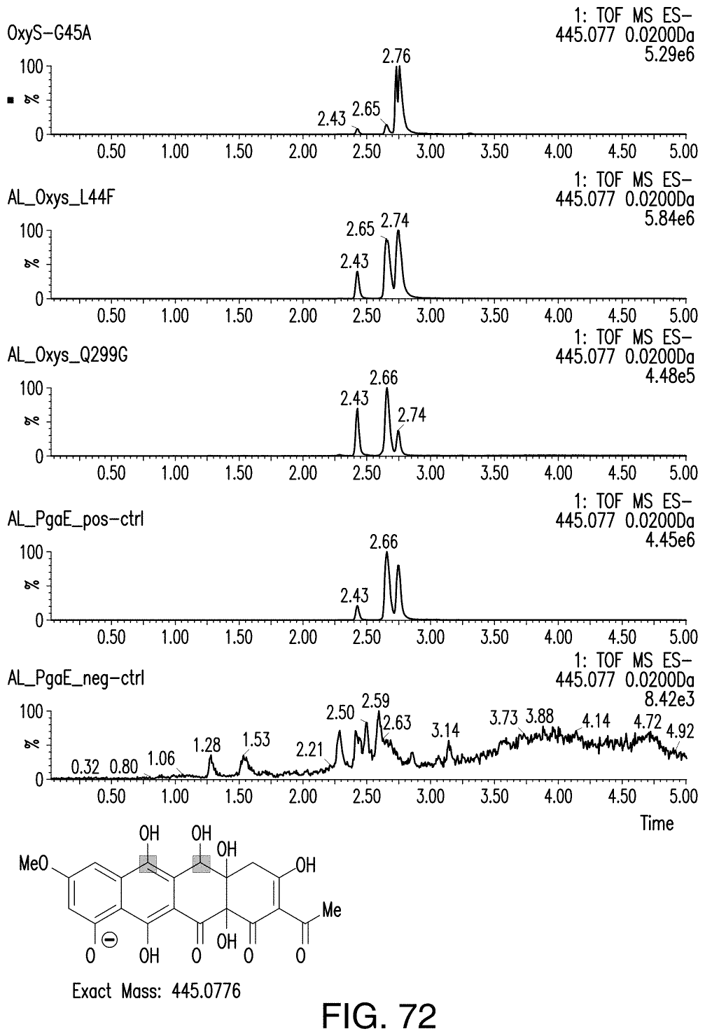

In certain embodiments, a genetically-engineered cell of the present disclosure can include one or more enzymes involved in the chemical modification of TAN-1612 to synthesize an analogue of TAN-1612 or to synthesize tetracycline or an analogue thereof by the addition of one or more hydroxyl groups. For example, but not by way of limitation, a mutant form of OxyS can be expressed in a cell for the generation of an analogue of TAN-1612, as described in Example 13. As disclosed in Example 13, wild-type OxyS shows no detectable activity with TAN-1612 as a substrate but mutant forms of OxyS disclosed herein can modify the 5 and/or 6 positions of TAN-1612, e.g., by the addition of hydroxyl groups to the 5 and/or 6 positions of TAN-1612 (see FIG. 72). For example, but not by way of limitation, a genetically-engineered cell of the present disclosure can be modified to express the enzymes for synthesizing TAN-1612, i.e., NpgA, AdaA, AdaB, AdaC and AdaD, in combination with an OxyS mutant disclosed herein. Amino acid and nucleotide sequence of non-limiting examples of OxyS mutants are disclosed in Tables 36-40. In certain embodiments, OxyS can be mutated at one or more amino acids, two or more amino acids, three or more amino acids, four or more amino acids, five or more amino acids or six or more amino acids. In certain embodiments, OxyS can be mutated as amino acids K42, A43, L44, G45, L95, F96, M176, W211, F212, T225, A227, F228, V240, P295, A296, G297, G298, G299, N302, I353, D354, R358, V372, P375 or a combination thereof. Non-limiting mutations at these amino acids include K42X, A43X, L44X, G45X, L95X, F96X, M176X, W211X, F212X, T225X, A227X, F228X, V240X, P295X, A296X, G297X, G298X, G299X, N302X, I353X, D354X, R358X, V372X and P375X, where X is any amino acid except for the wild type amino acid residue. In certain embodiments, the mutation at these amino acids include A43C, L44T, L44F, G45A, Q299S, Q299L and/or P357R. In certain embodiments, amino acid L44 can be mutated to a T or F amino acid. In certain embodiments, amino acid F288 can be mutated to an L or R amino acid. In certain embodiments, amino acid Q299 can be mutated to an S or L amino acid.

In certain embodiments, a mutated OxyS for use in the present disclosure can be mutated at one or more, two or more or at all three amino acids L44, G45 and Q299. In certain embodiments, a mutated OxyS for use in the present disclosure can be mutated at amino acids L44 and G45. In certain embodiments, a mutated OxyS for use in the present disclosure can be mutated at amino acids L44 and Q299. In certain embodiments, a mutated OxyS for use in the present disclosure can be mutated at amino acids G45 and Q299. In certain embodiments, a mutated OxyS for use in the present disclosure can be mutated at amino acids L44, G45 and Q299.

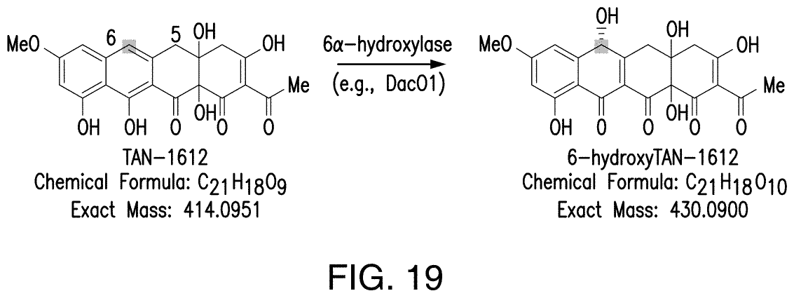

Additional enzymes that can be expressed in a genetically-engineered cell of the present disclosure to modify TAN-1612 or an analogue thereof with one or more hydroxyl groups include PgaE, SsfO1, CtcN and DacO1 as shown in FIGS. 19, 33, 61 and 71. In certain embodiments, the enzyme can result in a hydroxyl group at the 6α position, e.g., SsfO1 and DacO1. In certain embodiments, the enzyme can result in a hydroxyl group at the 6β position, e.g., CtcN. For example, but not by way of limitation, PgaE can be expressed in a cell to add a hydroxyl group to the 6 position of TAN-1612. In certain embodiments, a genetically-engineered cell of the present disclosure can be modified to express the enzymes for synthesizing TAN-1612, i.e., NpgA, AdaA, AdaB, AdaC and AdaD, in combination with PgaE.

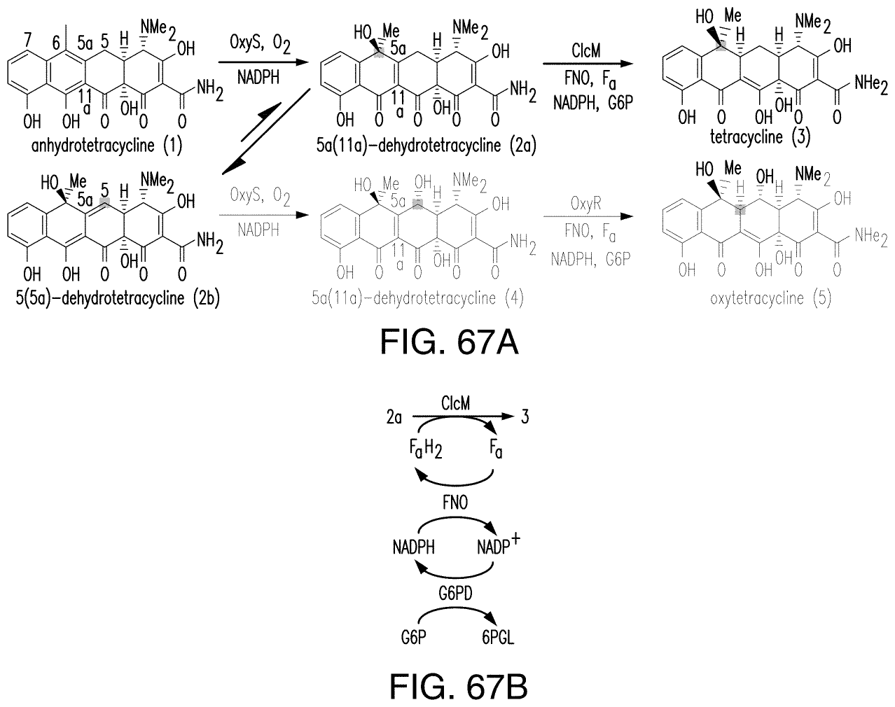

In certain embodiments, a genetically-engineered cell of the present disclosure can include one or more enzymes involved in the modification of TAN-1612 to synthesize tetracycline or an analogue thereof. For example, but not by way of limitation, a genetically-engineered cell of the present disclosure can include one or more enzymes selected from OxyS, OxyR, CtcM and FNO as shown in FIGS. 2 and 67. In certain embodiments, a genetically-engineered cell of the present disclosure can be modified to express the enzymes for synthesizing TAN-1612, i.e., NpgA, AdaA, AdaB, AdaC and AdaD, in combination with OxyR, CtcM and/or FNO to synthesize tetracycline.

In certain embodiments, a genetically-engineered cell of the present disclosure can include one or more enzymes involved in the chemical modification of TAN-1612 or an analogue thereof at the 2 or 4 positions. For example, but not by way of limitation, the genetically-engineered cell can be modified to express an oxygenase (e.g., OxyE) and/or an oxidase (e.g., OxyL) (see FIG. 61).

In certain embodiments, the one or more proteins, e.g., one or more enzymes, that play a role in the synthesis of TAN-1612, tetracycline or an analogue thereof can be expressed in the genetically-engineered cell as a fusion protein. For example, but not by way of limitation, a fusion protein comprising a reductase or a functional fragment thereof and a hydroxylase or a functional fragment thereof can be expressed in a cell. Alternatively or additionally, a fusion protein comprising a first reductase or a functional fragment thereof and a second reductase or a functional fragment thereof can be expressed in a cell. In certain embodiments, a fusion protein comprising a first hydroxylase or a functional fragment thereof and a second hydroxylase or a functional fragment thereof can be expressed in a cell. In certain embodiments, a fusion protein can include OxyS, OxyR, DacO1, DacO4, PgaE, SsfO1, CtcN, DacJ, DacM2 or functional fragments thereof. Non-limiting examples of such fusion proteins are provided in Table 4. For example, but not by way of limitation, the fusion protein can be DacO1-DacO4, OxyS-DacO1 or OxyS-OxyR. In certain embodiments, the fusion gene can include a protein, e.g., one or more enzymes, that play a role in the synthesis of TAN-1612, tetracycline or an analogue thereof, or a functional fragment thereof and a cytokine, e.g., Interferon-γ (IFNG) and/or ubiquitin (UBI). For example, but not by way of limitation, the coupling of IFNG and/or UBI to an enzyme disclosed herein, e.g., a fusion protein comprising the enzyme and IFNG and/or UBI, can result in the increased expression of the enzyme, e.g., the fusion protein, compared to the expression of the enzyme alone, e.g., in the absence of IFNG and UBI. Non-limiting examples of such fusion proteins include UBI-IFNG-DacO1 and IFNG-DacO1. In certain embodiments, the fusion protein is encoded by a nucleotide sequence disclosed in Table 5. In certain embodiments, the fusion protein is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 5.

In certain embodiments, one or more of enzymes, e.g., Fo synthase Thermobifida fusca, Fo synthase Chlamydomonas reinhardtii, FGD1 from Mycobacterium tuberculosis, F₄₂₀-dependent NADP+ oxidoreductase from Archeoglobus fulgidus, NADPH-dependent F₄₂₀ reductase from Streptomyces griseus, PgaE, SsfO1 and CtcN, can be heterologously expressed in a cell. In certain embodiments, one or more heterologous glycotransferase genes can be expressed in a cell. In certain embodiments, one or more heterologous fused hydroxylase and reductase genes can be expressed in a cell. In certain embodiments, one or more heterologous fused hydroxylase and reductase and glycotransferase genes can be expressed in a cell.

In certain embodiments, the one or more proteins, e.g., one or more enzymes, that play a role in the synthesis of TAN-1612, tetracycline or an analogue thereof can be modified by directed evolution to accept different small molecule, e.g., TAN-1612, as substrates.

In certain embodiments, a cell for use in the present disclosure can be genetically engineered to express an efflux pump. Efflux pump are transmembrane proteins, e.g., located in cytoplasmic membranes of cells, that transport compounds, such as antibiotics, out of cells. In certain embodiments, an efflux pump can be used to release a molecule, e.g., a therapeutic molecule, e.g., a peptide and/or a small molecule, from a genetically-engineered cell to treat a subject. Alternatively or additionally, the presence of an efflux pump in a genetically-engineered cell disclosed herein can be used to reduce the intracellular amount of a molecule of interest and/or reduce the toxicity associated with the generation of a molecule of interest, e.g., an antibiotic. For example, but not by way of limitation, the cells for use in the present disclosure can include an efflux pump derived from a fungal cell. In certain embodiments, the cells for use in the present disclosure can include an efflux pump derived from A. niger. Non-limiting examples of an efflux pump derived from A. niger include ASPINDRAFT 1768333, 185231, 43349 or 48051. In certain embodiments, the efflux pump is encoded by a nucleotide sequence disclosed in Table 1.1. For example, but not by way of limitation, the efflux pump is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 1.1.

Several pathways known to occur in yeast can be utilized to generates molecules, e.g., therapeutic molecules, of the present disclosure. Non-limiting examples of such pathways include carbohydrate metabolism pathways, e.g., glycolysis/gluconeogenesis, citrate acid cycle (TCA cycle), pentose phosphate pathway, pentose and glucuronate interconversions, fructose and mannose metabolism, galactose metabolism, ascorbate and aldarate metabolism, starch and sucrose metabolism, amino sugar and nucleotide sugar metabolism, pyruvate metabolism, glyoxylate and dicarboxylate metabolism, propanoate metabolism, butanoate metabolism, c5-branched dibasic acid metabolism, or inositol phosphate metabolism; energy metabolism, e.g., oxidative phosphorylation, photosynthesis, photosynthesis-antenna proteins, carbon fixation in photosynthetic organisms, carbon fixation pathways in prokaryotes, methane metabolism, nitrogen metabolism or sulfur metabolism; lipid metabolism, e.g., fatty acid biosynthesis, fatty acid elongation, fatty acid degradation, synthesis and degradation of ketone bodies, cutin, suberine and wax biosynthesis, steroid biosynthesis, primary bile acid biosynthesis, secondary bile acid biosynthesis, steroid hormone biosynthesis, glycerolipid metabolism, glycerophospholipid metabolism, ether lipid metabolism, sphingolipid metabolism, arachidonic acid metabolism, linoleic acid metabolism, alpha-linolenic acid metabolism, biosynthesis of unsaturated fatty acids; nucleotide mechanism, e.g., purine metabolism or pyrimidine metabolism; amino acid metabolism, e.g., alanine, aspartate and glutamate metabolism, glycine, serine and threonine metabolism, cysteine and methionine metabolism, valine, leucine and isoleucine degradation, valine, leucine and isoleucine biosynthesis, lysine biosynthesis, lysine degradation, arginine biosynthesis, arginine and proline metabolism, histidine metabolism, tyrosine metabolism, phenylalanine metabolism, tryptophan metabolism, phenylalanine, tyrosine and tryptophan biosynthesis, metabolism of other amino acids, such as, e.g., beta-alanine metabolism, taurine and hypotaurine metabolism, phosphonate and phosphinate metabolism, selenocompound metabolism, Cyanoamino acid metabolism, D-glutamine and D-glutamate metabolism, D-arginine and D-ornithine metabolism, D-alanine metabolism, or glutathione metabolism; glycan biosynthesis and metabolism, e.g., N-Glycan biosynthesis, various types of N-glycan biosynthesis, mucin type O-glycan biosynthesis, mannose type O-glycan biosynthesis, other types of O-glycan biosynthesis, glycosaminoglycan biosynthesis-chondroitin sulfate/dermatan sulfate glycosaminoglycan biosynthesis-heparan sulfate/heparin, glycosaminoglycan biosynthesis-keratan sulfate, glycosaminoglycan degradation, glycosylphosphatidylinositol (GPI)-anchor biosynthesis, glycosphingolipid biosynthesis-lacto and neolacto series, glycosphingolipid biosynthesis-globo and isoglobo series, glycosphingolipid biosynthesis-ganglio series, lipopolysaccharide biosynthesis, peptidoglycan biosynthesis, other glycan degradation, lipoarabinomannan (LAM) biosynthesis, or arabinogalactan biosynthesis-mycobacterium; or metabolism of cofactors and vitamins, e.g., thiamine metabolism, riboflavin metabolism, vitamin B6 metabolism, nicotinate and nicotinamide metabolism, pantothenate and CoA biosynthesis, biotin metabolism, lipoic acid metabolism, folate biosynthesis, one carbon pool by folate, retinol metabolism, porphyrin and chlorophyll metabolism, or ubiquinone and other terpenoid-quinone biosynthesis. In certain embodiments, the TCA cycle and/or the glycolysis pathway are utilized to generate molecules, e.g., therapeutic molecules, in a genetically-engineered cell of the present disclosure. For example, the TCA cycle and/or the glycolysis pathway can be utilized to synthesize tetracycline and analogues thereof, including but not limited to TAN-1612, by synthesizing precursors acetyl-CoA and malonyl-CoA, as discussed above. In certain embodiments, proteins, e.g., enzymes, that play a role in these pathways can be overexpressed and/or reduced in the genetically-engineered cell to enhance the metabolic flux towards AdaA. As shown in FIG. 58, enhanced expression of ALD6 (acetaldehyde dehydrogenase), ADH2 (alcohol dehydrogenase), acsSE (acetyl-CoA synthetase) and/or ACC1 (acetyl-CoA carboxylase) can result in the enhanced synthesis of the precursors acetyl-CoA and/or malonyl-CoA for the synthesis of TAN-1612, tetracycline or analogues thereof. In certain embodiments, a genetically-engineered cell of the present disclosure can include increased expression of ALD6, ADH2, acsSE and/or ACC1 as compared to a wild-type cell, e.g., by transforming a cell with one or more nucleic acids that encode ALD6, ADH2, acsSE and/or ACC1.

In certain embodiments, nucleic acids of the present disclosure encoding one or more of the therapeutic molecules and/or encoding one or more of the disclosed enzymes can be introduced into cells, e.g., yeast cells, using vectors, such as plasmid vectors and cell transformation techniques such as electroporation, heat shock and others known to those skilled in the art and described herein. In certain embodiments, the genetic molecular components are introduced into the cell to persist as a plasmid or integrate into the genome. For example, but not by way of limitation, the nucleic acid can be incorporated into the genome of the genetically-engineered cell. In certain embodiments, the cells can be engineered to chromosomally integrate a polynucleotide of one or more genetic molecular components described herein, using methods identifiable to skilled persons upon reading the present disclosure. In certain embodiments, a nucleic acid encoding a molecule of the present disclosure, e.g., peptide, can be inserted into the genome of a genetically engineered cell using homologous recombination. In certain embodiments, a nucleic acid encoding a molecule of the present disclosure, e.g., peptide, can be inserted into the genome of a genetically engineered cell using a CRISPR/Cas9 system.

In certain embodiments, a nucleic acid encoding one or more of the therapeutic molecules and/or encoding one or more of the disclosed enzymes can be introduced into cells is introduced into the yeast cell either as a construct or a plasmid. In certain embodiments, a nucleic acid can comprise one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid encoding a molecule of the present disclosure, e.g., peptide and/or protein, is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1, pFus1, GAP, P GCW14 and variants thereof. In certain embodiments, a variant of Tef1 is scTef1. In certain embodiments, a nucleic acid can include a constitutively active promoter, e.g., pTdh3. In certain embodiments, a nucleic acid can include an inducible promoter, e.g., pFus1 or pFig1. In certain embodiments, a nucleic acid can include a constitutively active promoter, e.g., pAdh1. In certain embodiments, a nucleic acid can include a constitutively active promoter, e.g., pCyc1.

In certain embodiments, a nucleic acid encoding one or more of the therapeutic molecules and/or encoding one or more of the disclosed enzymes can further include a transcription factor for regulation expression of the molecule encoded by the nucleic acid. Alternatively and/or additionally, a second nucleic or an additional nucleic acid can be introduced into the cells to express a transcription factor for regulation expression of the molecule encoded by the nucleic acid. Non-limiting examples of such transcription factors include Abf1p, Aca1p, Ace2p, Adr1p, Aft1p, Aft2p, Arg80p, Arg81p, Arr1p, Ash1p, Azf1p, Bas1p, Cad1p, Cat8p, Cbf1p, Cha4p, Cha4p, Cin5p, Com2p, Crz1p, Cst6p, Cup2p, Dal80p, Dal81p, Dal82p, Ecm22p, Fkh1p, Fkh2p, Flo8p, Fzf1p, Gal4p, Gat1p, Gcn4p, Gcr1p, Gis1p, Gln3p, Gon3p, Gsm1p, Gzf3p, Haa1p, Hacip, Hap 1p, Hap2p, Hap3p, Hap4p, Hap5p, Hcm1p, Hot1p, Hsf1p, Ime1p, Ino2p, Ino4p, Ino4p, Ixr1p, Kar4p, Leu3p, Lys14p, Mac1p, Ma163p, Mbp1p, Mcm 1p, Met31p, Met32p, Met4p, Mig1p, Mig2p, Mig3p, Mot2p, Mot3p, Msn2p, Msn4p, Mss11p, Ndt80p, Nrg1p, Nrg2p, Oaf1p, Pdr1p, Pdr3p, Pdr1p, Pho2p, Pho4p, Pip2p, Ppr1p, Put3p, Rap1p, Rcs1p, Rds1p, Reb1p, Rfx1p, Rgt1p, Rim101p, Rlm1p, Rme1p, Rof1p, Rox1p, Rph1p, Rpn4p, Rtg1p, Rtg3p, Sfl1p, Sip4p, Skn7p, Sko1p, Smp1p, Stb4p, Stb5p, Stb5p, Ste12p, Stp1p, Stp2p, Sum1p, Swi4p, Swi5p, Tda9p, Tea1p, Tecip, Tye7p, Uga3p, Ume6p, Upc2p, Usv1p, War1p, Xbp1p, YER130c, YFL052w, YHR177w, YJL103C, YML081w, YPL230w, Yap1p, Yap3p, Yap5p, Yrr1p, Zap1p and Znf1p. In certain embodiments, a nucleic acid introduced into a genetically-engineered cell of the present disclosure includes one or more DNA binding domains for a transcription factor. In certain embodiments, the DNA binding domain is a zinc finger DNA binding domain. In certain embodiments, the zinc finger DNA binding domain is ZF43-8. In certain embodiments, the transcription factor comprises one or more domains from different proteins. For example, but not by way of limitation, a transcription factor for use in the present disclosure can include an inducer binding domain, e.g., a β-estradiol binding domain, e.g., derived from the human estrogen receptor, and/or a transcription activation domain, e.g., derived from VP64.

In certain embodiments, a nucleic acid encoding one or more of the therapeutic molecules and/or encoding one or more of the disclosed enzymes can be inserted into the genome of the cell, e.g., yeast cell. For example, but not by way of limitation, one or more nucleic acids encoding a molecule of the present disclosure, e.g., peptide and/or protein, can be inserted into the Ste2, Ste3 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, one or more endogenous genes of the genetically-engineered cells can be knocked out and/or mutated, e.g., knocked out by a genetic engineering system. Alternatively or additionally, one or more endogenous genes of the genetically-engineered cells can be replaced with a homolog from a different species. In certain embodiments, a genetically-engineered cell can be modified to include multiple copies of an endogenous gene to increase expression of the gene. Various genetic engineering systems known in the art can be used. Non-limiting examples of such systems include the Clustered regularly-interspaced short palindromic repeats (CRISPR)/Cas system, the zinc-finger nuclease (ZFN) system, the transcription activator-like effector nuclease (TALEN) system, use of yeast endogenous homologous recombination and the use of interfering RNAs.

In certain non-limiting embodiments, a CRISPR/Cas9 system is employed to knock out one or more endogenous genes in the genetically engineered cell. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9) and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule and referred to alternatively as chimeric) or modular (comprising more than one, and typically two, separate RNA molecules, such as a crRNA and a tracrRNA, which are usually associated with one another, for instance by duplexing).

In certain embodiments, a sequence homolog of a nucleotide sequence disclosed herein can be a polynucleotide having changes in one or more nucleotide bases that can result in substitution of one or more amino acids, but do not affect the functional properties of the polypeptide or protein encoded by the nucleotide sequence. Homologs can also include polynucleotides having modifications such as deletion, addition or insertion of nucleotides that do not substantially affect the functional properties of the resulting polynucleotide or transcript. Alterations in a polynucleotide that result in the production of a chemically equivalent amino acid at a given site, but do not affect the functional properties of the encoded polypeptide, are well known in the art.

In certain embodiments, a sequence homolog of a peptide, polypeptide or protein disclosed herein can be a peptide, polypeptide or protein having changes in one or more amino acids but do not affect the functional properties of the peptide, polypeptide or protein. Alterations in a peptide, polypeptide or protein that do not affect the functional properties of the peptide, polypeptide or protein, are well known in the art, e.g., conservative substitutions. It is therefore understood that the disclosure encompasses more than the specific exemplary polynucleotide or amino acid sequences and includes functional equivalents thereof.

The cells to be used in the present disclosure can be genetically engineered using recombinant techniques known to those of ordinary skill in the art. Production and manipulation of the polynucleotides described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego.

In certain embodiments, the genetically-engineered cells express and/or secrete a molecule, e.g., therapeutic molecule, at high levels as compared to previous known expression systems. For example, but not by way of limitation, the total titer of the therapeutic molecule produced by a genetically-engineered cell, e.g., a population of genetically-engineered cells, is between about 1 pg and about 10 g, e.g., 1 pg/L to about 10 g/L. In certain embodiments, the total titer of the therapeutic molecule, e.g., TAN-1612, can be from about 1 mg/L to about 100 mg/L.

TABLE 1-1
DNA Sequences of Ada and Efflux proteins.

| | |
|---|---|
| adaA | ATGTCTGCCCCTACGAAGCTGGTCTTT |
| | TTCGGAAACGAATTCCCCAACGATGAT |
| | CTCAAAGCCCTCTTCCGCGGTCTGCAC |
| | CGACACGGCAAGGACAGGCGCTTCCGC |
| | CAGCTGGCAACCTTCTTGGAGGAGTCC |
| | ACTCGCGTGCTTCAGAATGAAGTCGCC |
| | CAACTTCCCGAGCCGCTAAAAAAGCTG |
| | GTGCCTCACTTTGAGAACCTGATGCCC |
| | CTAACCGAGGTTGATTTCCGCCAGGGA |
| | CCTCTGGGAGCTGCTATGGAAAGCGCC |
| | CTCCTCACAATTCTCGAACTGGGAATG |
| | TTCATCGGCCACTACGAAGCCGAGGAG |
| | CGTGTCTGGGACCTCTCCGCAGATCGG |
| | GCCACCTTGGCTGGTTTGAGTATTGGC |
| | CTGCTCGCTGCTGCTGGTGTCGCTCTG |
| | TCTACTCACTTGGCTGAGGTGGTCCAG |
| | AACGGCGCTGAGTGTGTGCGGGTTTCT |
| | TTCCGCTTGGGAGTCTACGTCCACGAC |
| | ATCTCCCGCAAGCTCGAAGCTCCCCAG |
| | GCAGACGGCAGCTTGCTCAGCTGGGCT |
| | CATGTTGTCACCGGTGAGACAGCATCC |
| | GACCTGCAGGAGGAACTGTCGCGATAC |
| | AATACGGAGACTGGCACTCCCGAGCTG |
| | CTCAAGGTCTTTATCAGTGCCGCTGAT |
| | AAGACCTCTGTGAGTGTCAGTGGCCCT |
| | CCCTCGCGCATCCGTGCGGCCTTCCGG |
| | GCCTCTCAGCGCCTGCGCTACTCCAAG |
| | TCTCTCGCCCTGCCCGTGTATGACGGC |
| | CTCTGCCATGCCGCCCATCTATACGAC |
| | GAGGAGACTATCCACCGTGTTCTTCAT |
| | CCAGATGGATCCGTCATCCCTACTTCC |
| | CGGCCGGTGCAACTGGCACTCCTCTCA |
| | TCGCGCTCTGGCCAGCCCTTCGAGGCC |
| | ACCACGGCCGCTGAGCTGTTCCGCGCC |
| | ATCAGCACAGAACTGCTGACTGGCACC |
| | ATCTTCTTGGACAATATCACGGCCGGT |
| | ATTCTCGACCGCACTGAACGCTGTGCC |
| | GACGCCACACAGTGCCAGATCGAAACC |
| | TATCGCACTTCGCTGGTGTTCAAGGGT |
| | CTGCTGAAAGCTCTGGAGGCTTGCTTC |
| | CCCGATCGGACCATCAGCACTACCGAT |
| | CTCATCCCCTGGGTGTTCCAGGACTAT |
| | GGTGCGCGCCAGCCCAAGTCATGCGCA |
| | GACTCGAAGCTGGCCATCGTCGGCATG |
| | GCCTGCCGCATGCCCGGAGGTGCCAAT |
| | GATCTCGACCTCTTCTGGGAGCTTCTC |
| | GCACAGGGTCGTGATACGCATACGACG |
| | GTGCCTGCGGATCGCTTCGACCTCGAA |
| | ACACACTATGATCCGACCGGTGAGACA |
| | GAGAACGCCACACGCACGCCCTTTGGC |
| | AACTTCATTGACCAGCCGGGACTTTTC |
| | GATGCGGGATTCTTCAACATGTCTCCT |
| | CGCGAGGCCGAGCAGACCGATCCCATG |
| | CACCGCCTCGCCCTTGTCACAGCATAC |
| | GAAGCCCTAGAAATGGCGGGTATCGTT |
| | TCTGGCCGCACGCCATCATCCAACCCT |
| | AAGCGCATCGCCACCTTCTACGGACAG |
| | GCCAGTGACGACTGGCGCGAGCTTAAT |
| | GCCTCCCAGAATATCGGCACCTACGCC |
| | GTCCCTGGTGGTGAACGTGCCTTTGCC |
| | AACGGTCGCATTAACTACTTCTTCAAG |
| | TTTGGTGGCCCATCTTTCAATCTCGAC |
| | ACGGCTTGCTCCAGCGGTCTGGCTGCC |
| | GTCCAGGCGGCCTGCTCTGCTCTGTGG |
| | GCTGGTGAAGCCGACACTGTCCTGGCC |
| | GGTGGACTGAACATTATCACCGACCCC |
| | GACAACTACGCCGGTCTGGGCAATGGC |
| | CACTTCCTGTCGCGCACCGGCCAGTGC |
| | AAGGTTTGGGACCAGTCTGCTGACGGT |
| | TACTGCCGTGCAGATGGTGTGGGATCT |
| | GTGGTCATCAAGCGCCTTGAGGACGCG |
| | GAGGCTGACAATGACAACATCCTGGCT |
| | GTCGTGCTCTCTGCTGCCACCAACCAC |
| | TCGGCGGAGGCCATCTCTATCACTCAC |
| | CCCCATGCTGGCGCCCAGAAGGAGAAC |
| | TACACCCAGGTGCTGCATCAAGCTGCC |
| | GTCAATCCACTGGACATAAGCTATGTC |
| | GAGCTGCACGGCACTGGCACCCAGGCC |
| | GGAGATGCCCAGGAGGCTGAGTCGGTC |
| | TTGGACATCTTTGCCCCACGAAACCAT |
| | CGTCGTCGCGCCGATCAGCCTCTGCAC |
| | TTGGGCGCGGTGAAAAGTAACATTGGT |
| | CATGGTGAGGCAGCTGCTGGTATTGCC |
| | TCCCTGCTCAAGGTGCTGCTCATGTAC |
| | CAGAAGAACGAGATTCCTGCGCACATC |
| | GGCATTCCCACAGTCATCAACCCGGCT |
| | ATCCCCACCGATCTTGAGCAGCGCAAG |
| | GTTTATTTGCCACGCACAAAGACTGCC |
| | TGGCCCCGGGCTGCAGGCCAGATCCGT |
| | CGCGCTATCGTGAACTCTTTTGGCGCG |
| | CACGGTGGTAATACTACCCTGGTGCTG |
| | GAAGATGCCCCTGAGAAGCAGGTGACG |
| | GTGGCCCGTGAGGAGCGCTCGACGCAC |
| | CCTGTCGTCATCTCCGCCAAGTCGAAG |
| | AAGTCCCTGGCCGCCAACGTGGAGACT |
| | CTCCTTGCCTACCTCGACGAGAACCCG |
| | GAGACTGACCTCGGCGACCTGTCCTAC |
| | ACGACCTGTGCCCGCCGCATGCACCAC |
| | AGCTGGCGTCTGGCCACTGCCGTCAGC |
| | GACATTCCCGCCCTGCAGAAGTTCCTG |
| | CGCAACGCTGTGAGCAACGATGCCGTT |
| | TCCCAGACCCGGCCCATTCCCACCGAG |
| | GCTCCCCCTGTCGTGTTCACTTTCACC |
| | GGCCAGGGCGCCTACTACGCCGGGCTG |
| | GCACAGGGTTTGTTCCAGGCTCTGCCT |
| | TTCTTCCGCGCCGAGGTGCGCCAGCTG |
| | GACCACCTGTCCCAGCGCCTGGGATTC |
| | CCCTCGATCGTGCCGGTCATCCTTGGC |
| | GAGGTGGAAGAGGGCACTGCTACGGCC |
| | CTGGTCACGCAGCTTAGCATTGTGATC |
| | GTGGAGATTGCCCTGGCCAGGCTCTGG |
| | CTACTCCTCCTAGGCATTCCGGCTCCT |
| | CACGCCGTGATCGGCCACAGTCTCGGC |
| | GAATACGCCGCATTGGCTGTCGCTGGA |
| | GTGCTGTCCACGGCAGACGCTCTCTAC |
| | CTGGTCGGCCACCGGGCTCAGCTCATT |
| | GAGGAACACTGCACCCCCGGCAGTCAC |
| | GCCATGCTCTCGGTGCGCGCAACCATC |
| | GCTGACATCGAGCGTCTGGTGGGCACC |
| | GGCTCTGATGCACCGACTTACGAGCTG |
| | TCCTGCCAAAACACGCACCAGGACACC |
| | GTCATCGGCGGCTCAATCCAGGACCTC |
| | AATGCCATCCGGGAGAAGCTCGAGCAT |
| | GAAGGAATCAAGTGTGTCAACGTCGAT |
| | GTCCCCTTTGCCTTCCACACCGCGCAG |
| | ATGGACGCCGTCCGGGAACGCCTCGCC |
| | AAGGCCGTCGCCGCCGTGCCCTTCAAG |
| | ACCCCCAGCGTCCCTGTTCTCTCGCCT |
| | CTGCTGGGCAGCGTCGTCTTTGACGGC |
| | AAGTCTATCAACCCAGAGTACATCGTG |
| | CGGGCAACTCGCGAACCCGTCCAGTTT |
| | GCGACTGCCATCGACGCCGCCCAGGAA |
| | CTCGGCATCGTCAACAGCCAGACCCTC |
| | TGGGTCGACATCGGCCCCCACCCCATC |
| | TGCGCCAGTTTCGTGCGCAGTCTGGTG |
| | CCTGGCGCACGCATCGTTTCGTCCTGC |
| | CGCCGTAACGAGGACAACTTTGCAACC |
| | ATGGCCAAGAGTCTCTGCACACTGCAC |
| | CTGGCCGGCCGCACCCCCTCCTGGGCC |
| | GAGTACTTCCGTCCCGACGAGCAGGCT |
| | TACTCTCTCCTCCGGCTGCCCAAGTAC |
| | CGCTGGAACGAGGTCAACTACTGGATC |
| | CAGTACCTGGGCACCTGGACCCTGGAC |
| | AAGGCGCACCTCAAGAACGGCGGCAGT |
| | CAGAAGCGCGCCATCACCGATGTACCA |
| | TCTATCTCATCCCTCCGCACCTCCCTC |
| | ATCCACCAGGTCACCGAGGAGACTGTC |
| | GACAAGACCACCGCCACCCTCAAGGCC |
| | ATCTCCGACATCCAGCACCCCGACTTC |
| | CTCGAAGCTGTGCACGGCCACACCATG |
| | AACAACTGCGGCGTCGCCACCTCCTCC |
| | ATCTGGACCGACATGGCCATGACCGTC |
| | GGCGAGCACCTCTACCGTCGCTTGGTC |
| | CCTGGCACTGACCACGTCCTCATGGAC |
| | CTCTGCGACTTTGAAGTCCAGCACGCT |
| | CAAGTCGCCAACACCAACTCAAACACC |
| | CCGCAACCCCTCGCCCTCGAAGCCCAC |
| | CTCGACCTCCCCACCCGCCACATGTCC |
| | CTCGCCTGGTACGACGTCAATGCCACG |
| | ACCAACCAGCGCGCCGATGCCCCCTTC |
| | GCCACCGGCAGCATCAAGTACCCTGCC |
| | GACCCAACCGGCGCAGCCTGGTCCATC |
| | GAATGGTCCCGCATCACGCACCTCATT |
| | CAGGGCCGCATCGAAGCCCTGCAGCAC |
| | CTGGCCGCGGAGAACAAAGCCAGCACT |
| | CTGTCCAAACCCCTCGCCTACGCTCTC |
| | TTCAAGAACGTCGTCGACTACGCCCCC |
| | CGCTACCGCGGCATGGACCGCGTTGTC |
| | ATTCACGACCACGAGGCCTTCTCCGAT |
| | ATCACCCTTACCACCGACCGCCACGGA |
| | ACCTGGCACACCCCGCCCCACTGGATC |
| | GACAGCGTTTCGCACCTGGCCGGCCTG |
| | GTCATGAACGGCAGCGATGCTTCCAAC |
| | ACCAGGGACTTCTTCTACGTCACCCCT |
| | GGCTGCAGCAGCTGCCGCATGGCTGAG |
| | CCTTTGATTGCTGGAGGCAAGTACCGG |
| | AACTATGTCCGCATGTTCCCCATGCCG |
| | GACGAGGCGCACATGTATGCGGGCGAC |
| | TTGTACATCCTCCGCGAGGACAAGATC |
| | ATTGGTGTCGTCGAACAGCTCAAGTTC |
| | CGCAGAGTGCCCCGCTTGTTGATGGAT |
| | CGCTTCTTCTCGCCGAATAAGAATGCC |
| | GCGGCGCATGCTGCTCCTGCTCCTGCT |
| | CCTGCTGCGGTTCCAGCCGTGAAGAAG |
| | CAACCTCCCACTGAAACCATCCAGCCC |
| | CAGGCCCCCAAGACAGAGCAGAAGCAA |
| | GACCAGCTACAACTTCCCAATCTCGCC |
| | TCCGCTGCACCCTCCACCGCCAGCAGC |
| | TCATCCTCACCCTCATCCAGCGGCGTC |
| | GCTACCCCCACTACTGAGCAAGAAGCC |
| | CCTGGCGCTGATGCCTCAGCGGTGACC |
| | GGCGTGGCCGGCAAATGTCTGGAACTG |
| | ATTGCCAACGAGACAGGCCTGGGTGTG |
| | GCGGAATTGACTGCGGACGCGACATTC |
| | GTGCAGCTGGGTGTGGATTCGCTGATG |
| | TCGCTGGTGCTGTCGGAGAAGTTGAGA |
| | AGTGAGATGGGATTGGAGATTAAGAGT |
| | TCGTTGTTCTTGGAGTGTCCTACTGTG |
| | GGAGATTTGACGGGGTGGTTGGAGCAG |
| | TATTGCTAA (SEQ ID NO: 1) |
| adaB | ATGGCCTTCCGTATCCCCTTCGCTCAA |
| | TCTTTCTGGCAAGAATACCTGTCCGGC |
| | CAGGAAGCCAACCTGCCTCGTCTACCC |
| | GAAGTCGAGCAAGTCACCGAGACAGTA |
| | ATGCGCATTCTCGGAGGTAATCCCGGG |
| | CGCATGCAGCTGCAAGGGACCAACACG |
| | TACCTCGTGGGGACGGGCAAATTCCGA |
| | ATTCTCATTGACACCGGCCAGGGTGAA |
| | GCAAGCTGGATCGAAGCCCTCACGAAA |
| | CAACTTGAAGCCAATGGTCTAGAAATT |
| | TCCCATGTCTTGCTCACTCACTGGCAC |
| | GGAGATCACACCGGAGGGGTCCCCGAC |
| | CTCATCACCTACAACCCCGAACTATCC |
| | TCACGAGTATACAAAAACACCCCCGAT |
| | CTCGGCCAACAAGCCATCCACGATGGT |
| | CAGAAATTCCACGTAGAGGGCGCAACT |
| | ATCCGTGCTGTCTTTACCCCCGGCCAC |
| | GCCTTTGATCACATGTGCTTCCTGCTG |
| | GAGGAAGAAAATGCCCTCTTCACTGGG |
| | GACAACGTGCTAGGACATGGATACTCT |
| | GTCGTGGAAGATCTGGGCACATATATG |
| | ACCAGTCTAACACGCATGGCGGACCTC |
| | AACTGCGCGCTTGGGTATCCAGCTCAT |
| | GGGACGCGTATCGAGGATCTTCCTGCC |
| | AAGATGAAGGAATATATCCAGCATAAG |
| | GAGTCAAGGATGCGGCAGGTGCTGGCC |
| | GCCTTGGAGAGGAGTCGGGCGAGGATG |
| | ACGGCAACTGGGGGTGGGCGCCGTGCG |
| | GGTGCGCTGACATTCCCGGAGCTTATA |
| | AACTCTATGTATGGAGGGATTCCAGAT |
| | GAGATTGAACAGGCTTTGACGCCGTTC |
| | TTGAGTCAGGTTTTGTGGAAATTGGCG |
| | GAGGATCGGAAGGTTGGGTTTGAGGGC |
| | GAGCCGAGTCAGAGAAGATGGTTTGCG |
| | GTTGGGCCGCCAGCTGCAACTGCAGTG |
| | AGGCTGTAA (SEQ ID NO: 2) |
| adaC | ATGACCCCTCCCATCCTCATCATCGGC |
| | GCCGGCCTCTCCGGCCTCACCATCTCC |
| | CGCATCCTCACCAACGCCTCCATCCCC |
| | AACATCGTCTTTGAAGCCTCCACCCCC |
| | GACCGCAGCCAAGGCTACGCCATCAGC |
| | CTCCGCGAATGGGGCTACACCTCCCTC |
| | CTCACAGCCCTAGGTGACCTGCCCCTC |
| | CGCAGTCTCACCCGGGGCGTGGCTCCA |
| | GACCGCATCCTCGGCGGAACCGGCTGG |
| | ATCGACCAAGCCCTCCGGGACAACCAC |
| | ACCGGAAACCTCCTAGTGGCTCCCGAC |
| | CCAGAAGCCAAGCAATGCATCGTGCGG |
| | GCAAATCGCAACGCCCTCCGCACCTGG |
| | ATCGCCGATAGCGGTGACGAGGAAGTG |
| | GACATCCGCTACGGCCACCGTCTGCGC |
| | AGTGTGCAGGGCTCAATGGGAAACGTG |
| | ACGGCCACGTTCGACAACGGCGCCAAG |
| | TACCAGGGCTCATTGGTCATCGCAGCA |
| | GATGGCGTGCATTCTAGCGTGCGTTCT |
| | CAGATCCTGCCCCACGTGAGCCCGGAT |
| | ATTGTGCCCGTGGTGGTGTATCATGGG |
| | GAATTGGAACTCCCTCGCAAGGAATTC |
| | GATAATCTTATCCGTCCGCACTCCGGA |
| | CCGTCGAATATCCTAGCTGGTGTGGGC |
| | GATGGGTTCAATACCCCTATTACTGTT |
| | TGTAATATCACTCCTACACATGTGCAT |
| | CTCGATTGGTCGTATTCTAGACCGAGT |
| | ACGGAAAACAAGGAGAATAAGGATCCT |
| | CTCTATCGGCCGCATGTCTCTGCTGCT |
| | GAGGCTAAGCAGATTCCCCCGGCCTTG |
| | TTGGAGGAAATTGCCTCCCGGGATCTG |
| | GCGAGGCCGTGGTCCCAGCTGCTTAAT |
| | GCCGAGGCGTTGCCGACTCATCGTGTC |
| | TTTAACTGGGTGAGTCGCTGTGTGTCG |
| | GTAACGAGGGAGGATGTGAATGCTGCG |
| | CAGAAGCAGGGCGTGGTGTTTATTGGG |
| | GACTCGTGGCATGCGATGCCAATTTTT |
| | GGGGGCGAAGGGGGTAATCATGCCCTG |
| | GTTGATGCGGTGGAGTTGGCGGAGGCA |
| | TTGACGGGTAAGGAAGGGAATTTGGAT |
| | GCTGCTGTAACGGGGTATTATGATCGC |
| | GCGTGGAGAAGATGTCAGGAGGCCGTG |
| | AGAAGATCTAGGCAGAGGTTCTTCCAG |
| | TTGCATCGGCCTATGAGGGAGTGGATG |
| | GAGATTGCGGAGAAGAAAAAGATGATG |
| | GCTGCTATGAAGGGGGTGGAGGCTCAT |
| | TAA (SEQ ID NO: 3) |
| adaD | ATGTCCTCAGTCACCTTAACCACAACC |
| | ACAACCACCACCTCCACCCCACCCAAA |
| | CCCACCCCCAAAGACGAACCCCAAGAA |
| | CAAATCTACACCCCCTGGCGCCTCTTC |
| | ATCTACGACATCTGGGTCCTCGGCATC |
| | GTGAGCACCCTCGCCTGGGGCTGCCGG |
| | ATCAGCACCTACCTAATCCCCTTATTC |
| | CGCTCCAACGTGGGCAAAAAGCACCTC |
| | GACATCGGCGCCGGCACCGGCTACTAC |
| | CTCAACCAAGCCCGGATCTCCTCAACA |
| | ACCCAGCTCACAATCGTCGACAACGAA |
| | ACCCACGCGCTCAACGTCGCCCTCGCT |
| | CGCTGCAAGCACCCAGTCACCCAAACG |
| | CACGGCATCGTCACCGACATCCTGCAA |
| | CCCTCGCCCTTCCCAGAAACCTACCTC |
| | ACCAACAATGACCAAAAATTCGACTCC |
| | GTATCAATGTACTACCTCCTGCACTGC |
| | CTGCCCGTGCCCGTGGCCAGCAAATGC |
| | AAGATCTTCACGCATCTCAAGAAATAC |
| | ATGACAGAGGATGGGGTGGTTCATGGA |
| | GCGAATGTGCTGGGCAAGGGGGTGAGG |
| | AAGGATAATTGGTTTGCGAGGATTATC |
| | CGAAGAGGGTGTTTGAATCATGGGGTG |
| | TTCCATAATGAGGAGGATAATGCGTAT |
| | GAGTTTGAGAGGGCCCTCAGGGAGAAC |
| | TTTTGGGAGGTGGAGACTTGGGTGGTC |
| | GGGAGTGTTTTTGTTTTTAGGGCCAAG |
| | AGGCCGATTCTTGATGCTTAA |
| | (SEQ ID NO: 4) |
| NpgA | ATGGTGCAAGACACATCAAGCGCAAGC |
| | ACTTCGCCAATTTTAACAAGATGGTAC |
| | ATCGACACCCGCCCTCTAACCGCCTCA |
| | ACAGCAGCCCTTCCTCTCCTTGAAACC |
| | CTCCAGCCCGCTGATCAAATCTCCGTC |
| | CAAAAATACTACCATCTGAAGGATAAA |
| | CACATGTCTCTCGCCTCTAATCTGCTC |
| | AAATACCTCTTCGTCCACCGAAACTGT |
| | CGCATCCCCTGGTCTTCAATCGTGATC |
| | TCTCGAACCCCAGATCCGCACAGACGA |
| | CCATGCTATATTCCACCCTCAGGCTCA |
| | CAGGAAGACAGCTTCAAAGACGGATAT |
| | ACCGGCATCAACGTTGAGTTCAACGTC |
| | AGCCACCAAGCCTCAATGGTCGCGATC |
| | GCGGGAACAGCTTTTACTCCCAATAGT |
| | GGTGGGGACAGCAAACTCAAACCCGAA |
| | GTCGGAATTGATATTACGTGCGTAAAC |
| | GAGCGGCAGGGACGGAACGGGGAAGAG |
| | CGGAGCCTGGAATCGCTACGTCAATAT |
| | ATTGATATATTCTCGGAAGTGTTTTCC |
| | ACTGCAGAGATGGCCAATATAAGGAGG |
| | TTAGATGGAGTCTCATCATCCTCACTG |
| | TCTGCTGATCGTCTTGTGGACTACGGG |
| | TACAGACTCTTCTACACTTACTGGGCG |
| | CTCAAAGAGGCGTATATAAAAATGACT |
| | GGGGAGGCCCTCTTAGCACCGTGGTTA |
| | CGGGAACTGGAATTCAGTAATGTCGTC |
| | GCCCCGGCCGCTGTTGCGGAGAGTGGG |
| | GATTCGGCTGGGGATTTCGGGGAGCCG |
| | TATACGGGTGTCAGGACGACTTTATAT |
| | AAAAATCTCGTTGAGGATGTGAGGATT |
| | GAAGTTGCTGCTCTGGGCGGTGATTAC |
| | CTATTTGCAACGGCTGCGAGGGGTGGT |
| | GGGATTGGAGCTAGTTCTAGACCAGGA |
| | GGTGGTCCAGACGGAAGTGGCATCCGA |
| | AGCCAGGATCCCTGGAGGCCTTTCAAG |
| | AAGTTAGATATAGAGCGAGATATCCAG |
| | CCCTGTGCGACTGGGGTGTGTAATTGC |
| | CTATCCTAA (SEQ ID NO: 5) |
| ASPNIDRA | ATGGCCCGCCGGAACAGCATACGCGGG |
| FT_176833 | ATCACAGACGAGCAGCACGGCCGGTAT |
| | AGATCCAGCTCGCCGCTCGCCCGCACT |
| | GCCATCGCCCGCGATATTGAGGACTAC |
| | GCCGACGACGAAGGATCCATGCTCACC |
| | ACCGACGACGAAGCCTCCGAAACCTCC |
| | ACCATCCGCGCCATTAACAGCCAGCCC |
| | GGCACGAATCCCCATTCGCTGGCCGGG |
| | TCGTACCAGCGGCCCGGATTCTTCACT |
| | ACCGTATCGCACGCGACGGTTGTGCCG |
| | CATCGGCCGGATAACGAGGGGTTAACG |
| | CGGCGGGAGCGGGAGCGGGCCATTGAG |
| | GATGAGCGGAATCTTCTCACTGATAAT |
| | CGATGCATTGAGCCGGGTGTGCAGAAG |
| | GGAGGGCGCATGAGGCGCGGGTCGGGT |
| | GTGGAAGCGACGGAAACGGCTGCATTG |
| | TTGGGCGGGCAGCGTCGCGGATCACAG |
| | TATGAAACGGTTGAGGATCAGGAGGAG |
| | ATTGATCGGAAGTGGGAGGAGGCTGTT |
| | ACCGCTGGGTTGATTCAGACGACGTGG |
| | AAGCGTGAAGCTCAGGTCATTGGGAAG |
| | AATGCCGCGCCGTTGGTGGTTACCTTT |
| | CTGCTGCAGTATTCGTTGACCGTGGCG |
| | AGTATTTTTACCCTCGGGCATTTGGGC |
| | AAGAAGGAGCTTGGGGCCGTTAGTTTG |
| | GCTAGTATGAGTGCGAGTATTACGGGT |
| | TATGCTGTTTACCAAGGTCTGGCGACG |
| | AGTTTGGATACCCTGTGTGCGCAGGCG |
| | TATGGCTCCGGGAGGAAGAAGCTGGTA |
| | GGTCTGCAGATGCAAAAGATGGTATTC |
| | TTCCTCTGGGCTATCTCCATACCTATT |
| | ATTTTGTTGTGGTTCTTCGCCGACCGC |
| | ATCCTCGTCCGGATCGTGCCGGAAAGA |
| | GAGGTCGCCATGCTGGCGGGCTTGTAC |
| | TTGAAGGTGGTTGCGCTGGGTGCTCCA |
| | GGGTACGCTTGCTTCGAGAGTGGCAAG |
| | CGGTTCGTGCAGGCGCAGGGACTCTTC |
| | TCTGCCTCGCTTTATGTGCTTCTCATC |
| | TGTGCCCCGTTGAATGCAGTGATGAAC |
| | TACGTATTCGTATGGCAGTTCGGCTGG |
| | GGCTTCATCGGAGCTCCCATCGCTGTA |
| | GCCATCACAGACAACCTGATGCCTCTC |
| | TTCCTCTTCCTGTACGTGTACTTCATC |
| | GACGGCTCCGAATGCTGGAACGGCGTC |
| | ACCACCCGCGCCCTACGCAACTGGGGC |
| | CCCATGATCAAGCTCGCCCTTCCCGGG |
| | CTCCTCATGGTCGAAGCCGAATGTCTC |
| | GCCTTTGAAGTTCTGACCCTGGCGTCC |
| | TCGTATCTCGGCACCACCCCATTAGCC |
| | GCCCAATCCATCCTGTCCACCATCTCC |
| | AGCATCACCTTCCAAATCCCCTTCCCC |
| | GTATCCATCTCCGGTAGCACCCGCGTC |
| | GCCAACCTCATCGGCGCGACGCTTGTC |
| | GACGCCGCCAAGCTATCCGCCAAGGTA |
| | TCCATGATTGGCGCCGTCATCGTCGGG |
| | CTGCTGAACATGCTCCTCCTCTCTTCC |
| | CTGCGCTACTACATCCCGTACCTGTTC |
| | ACCTCGGACGAGGAGGTCATTGAGCTC |
| | GTCGCCCAGGTCCTGCCTCTCTGTGCG |
| | GCGTTCCAGCTCTTTGATGCCTTGGCT |
| | GCCAACTGTAACGGAATTCTGCGCGGT |
| | ATCGGTCGCCAGGAAATTGGCGGCTAT |
| | GTGCAGCTGTTTTGCTACTATGCTATT |
| | GCTATGCCAATTAGTTTCGGCACGACA |
| | TTTGGATTGAACTGGGGCTTGTTTGGA |
| | CTGTGGTCTGGTGTTGCGTTGGCCTTG |
| | TTATTGGTATCGGTCATTGAGGCGTTC |
| | TTTTTGACGCAGACGAATTGGCATCGC |
| | TCGGTGGA |
| | GGATGCGCTGCGCCGGAATGCGATGAC |
| | TTAA (SEQ ID NO: 6) |
| ASPNIDRA | ATGTACGACTCTCTTCCTAGCTACCGG |
| FT_185231 | GAGACTTCTTCCGCGCACACTCATGGA |
| | GAGGAACATACGCCACTACTTCCCAAG |
| | CAGGTCGACACCGGCCCCGACTCCAAA |
| | AGCGCAAAATCATCGGTTTCCTTTCTG |
| | GTCGAATTTTTCCGGCTGTTGAAGGAC |
| | TCCATACCAGTCATTCTGGCATACACG |
| | CTGCAAAACAGCCTACAAACCACCTCG |
| | GTGTTGATTGTGGGACGGACCTCTCCG |
| | GAGAACCTGGCCACCACAGCTTTCTCA |
| | CTGATGTTTGCCATGGTCACGGCATGG |
| | ATGATCGCCTTAGGCGGGACCACCGCA |
| | TTGGACACACTAGCCTCCTCAACGTTC |
| | ACAGGCAGCTCGAATAAGCATGATCTC |
| | GGAATTCTGTTGCAGCGGGCATTCTTT |
| | GTCCTCGGCCTGTTCTATGTCCCCGTT |
| | GCAATTCTTTGGACATGCTCGGAGCCT |
| | GTGTTTCTTCTGCTTGGCCAGGACCCC |
| | CAGCTTTCACGGGATAGTGCGCGTTTC |
| | CTAACTTGCTTGATCCCTGGCGGTCTG |
| | GGTTATATCTACTTCGAGGCCATGAAA |
| | AAATATCTGCAAGCACAGGGTATTATG |
| | CGCCCTGGGACGTATGTCTTGCTGATT |
| | ACGGTGCCGTTTAATGCATTGCTCAAT |
| | TACCTGTTCTGTTACACCTTTCGCATG |
| | GGGTTGCTTGGTGCGCCATTTGCCACC |
| | GGCATTTCCTACTGGCTGTCCTTTGCA |
| | CTGCTCGTGTTGTATGCCCGATTCATT |
| | GCCGGCTCCGAATGCTGGGGCGGCTGG |
| | TCGCGCAAAGCATTCGAGAATCTTGGG |
| | ACTTTCGCCCGATTGGCCTTTCTGGGC |
| | GTGGTGCATGTAGGCACCGAGTGGTGG |
| | GCCTTTGAGATAGTAGCTCTTGCAGCC |
| | GGAAGGCTGGGAACAATCCCACTGGCG |
| | GCTCAGAGCGTCATCATGACCGCAGAC |
| | CAAGTGCTCAACACAATCCCATTTGGC |
| | GTGGGAGTCGCTACATCTTCACGGGTG |
| | GGGAGCCTGCTAGGCTCCCGGGATGCA |
| | GCTGGTGCATCGAGAGCGGCCAACACC |
| | GCGGCCTGGCTAAGTATGGCGCTGGGA |
| | GGGGCCGTGCTGGCTGTGCTGATGGGG |
| | ACTCGACACGTCTTTGCAAAGATATTC |
| | AACTCCGATGAAGGGGTCGTACAGCTC |
| | ACTGCGGAGGTTCTACCATGGGTGGCG |
| | TTGTTCCAGATCGCCGATGGATTGAAC |
| | GGTAGCTGTGGAGGAAGCCTTAGGGGT |
| | ATGGGAAGGCAGCATGTCGGCGCGCTA |
| | GTCAACCTGGCCAGTTATTATTGTGGT |
| | GCACTACCTCTAGGTATTTGGCTAGCC |
| | TTCAATGGATGGGGACTGAAAGGTCTT |
| | TGGGTGGGACAGTGCATCGCATTGTAT |
| | CTTGTGGGGGCACTGGAGTGGACAATC |
| | GTGGCCTTCAGTAACTGGGAAGGCGAG |
| | GTCGATAAGGCGTTTCAGCGAATGGAT |
| | ATCCATGACCGATTGGAGGTTGGACAC |
| | ACCACCAATGGTGCCACGACTGTTGTG |
| | TAA (SEQ ID NO: 7) |
| ASPNIDRA | ATGACGGACGAGAAGAATGCCCGGGAG |
| FT_43349 | CTAACGGCCACCCCTGTCGACTTGGAT |
| | GAACTGCCGGCCGGATACTACTGCAGG |
| | CCAGACCCCAGCTACAGCTTGATCTCG |
| | ACCGTTTTCACACTGATCTCCGGAGTC |
| | GGTCTACTCTTGGTAGGCCGCTTAGGA |
| | GATCTCTTTGGCCGCCGATACATCCTG |
| | ATTGGAGGGCAACTACTTGGCCTTATC |
| | GGCGCCATAGTCTGTGCAACAGCCAAA |
| | ACCGTTCCCACCGTTATCGGAGGCAGT |
| | GTCCTCTGCGGTCTGGCTGCTGCAGTG |
| | CAATTGACTTTCAGCTTCGTGATCGCT |
| | GAGCTGGTCCCCAACCGTGCTCGTCCG |
| | GTCGTCAATGCTGGCATCTTCATCACA |
| | ACGCTGCCCTTCTCAGCGTTCGGTTCT |
| | CTGATTGCAGATCTGTTCATCGCCAAC |
| | ACCGCCCGCTCCTGGCGCTGGACTTAC |
| | TACCTGAACATCATCACCTGCGGTCTT |
| | TCTATCATTTTGCTGGTGCTGTTTTAC |
| | TTCCCTCCAGGCTGGGATGCCAAGCGC |
| | GGAAATGAGAGCCGCATCGATGGCCTC |
| | AAGAAATTCGACTACATTGGCTTTCTC |
| | CTATACGCCGGCGGACTGATCCTGGTC |
| | CTGCTTGGCCTCTGTGAGTCTCCCGTT |
| | CCAGCTCTTACTCGGTTTTTCCATGGT |
| | GCTAATTCAATCCCATCAGCATGGGGC |
| | GGCTCATCGTATGCATGGCACTCAGCC |
| | CATGTCATCGCTGTTCTGGTCATTGGA |
| | TTCGTCTGCTTGCTCGCTTTTGCCCTA |
| | TATGAAATATTCGTCCCCTTGCAGCAG |
| | CCTCTCCTGCCCATGTCCTTACTCCGA |
| | AACCGCGGATACGCCGGTGCGGTGTGC |
| | TCGGCCCTAGTAGGAAACATGGTCTAC |
| | TTCTCCATGAGTCTCCTCTGGCCAACC |
| | GCCATCGCCGACCTCTACACCACAAAC |
| | ACCATCAAAACGGGCTGGCTTTCAATC |
| | TCCACCGGCGCCGGCGTCATCGTCGGC |
| | GAAGTCGCCGCCGGCATCCTGATGAAA |
| | CCCGTGGGATACACAAAATACCAACTG |
| | ATCATCACCACCCTCATGATCACCGCC |
| | TTCTCCGGGGCCCTAGCATCCATTAAC |
| | CAATACCGCCAGGCATACGGCATCGCG |
| | TTCACCGCCCTCGGCGGCTTCTTCGTC |
| | GGCTACCTCGAACTGATAACCATAATC |
| | ATGTGCCCCCTGTATTGCTCACCCCAG |
| | GACATCGGGCTAGCCAGCGGCTTCCTC |
| | GGCTCCGCAAAGCAAGTCGCCGGCACC |
| | ATCGCCACCGCCATCTACGTCGCCATC |
| | CTGGACAACCGCGTCACGGTGGACTTG |
| | CCGCGCGATGTATCTGCTGCCGCATTG |
| | AACGCAGGACTGCCTTCATCCTCGCTG |
| | ACGGATCTGCTGGAGGCGGTGTCGGCG |
| | GGGACGACGGCAGCGTATGAGGCTGTT |
| | CCCGGCATGACGAATGGTATTCTGGGG |
| | GCTGTGACAGAGGCGGTGAAAACGGCG |
| | TATTCGCAGTCGTTTCGCACGGTGTTT |
| | TTGGCGAGTATTGCGTTTGGCGGACTG |
| | TCGGTGATCGCGGCGTGTTTTGCCGAG |
| | GGGGTGGATGGGAGGTTTACGGGCGAT |
| | GTGGCGGCTAGGTTGAAGGGTGTGGAT |
| | GGTGGTGGTTCTGGAAGTGAGGAGAAG |
| | TTGAAGGATGGGAAGGTTTAA |
| | (SEQ ID NO: 8) |
| ASPNIDRA | ATGGACGACCGCGAAAACCCCAAAAAC |
| FT_48051 | TGGAGCATCAGCAAGAAATGGGCTGTG |
| | ACCATCACCGTGTCGCTCTTCACATTC |
| | ATTTCCCCCGTTTCATCCTCCATGATC |
| | TCACCCGCCTTGACCAAGATCGCCAAA |
| | GAACTCAACATCACATCGCAAGTCGAA |
| | TCCGAACTAAGTCTCTCTGTTTTCGTG |
| | CTCGCCTACGCCGTCGGCCCACTCTGC |
| | TTCGGCCCGCTATCCGAACTCTTTGGT |
| | CGTGCCTACGTCCTGCAATGCAGTAAT |
| | CTATGCTACGTGGCGTGGAACCTTGGA |
| | TGCGGCTTCGCGCAGACATCGGGGCAG |
| | TTGATCGCGTTTCGTTTCATGAGTGGG |
| | ATAGCAGGTAGTGCGCCGTTAGCTATT |
| | GGAGGTGGTGTTCTCAGTGACTGCTGG |
| | AACGCCGACCAACGCGGCAAAGCCGTC |
| | GGCATCTACAGCCTGGCCCCCCTTCTC |
| | GGTCCCGTCGTGGGCCCCATCGCGGGT |
| | GGCTTCATCGCCGAAACGACGACCTGG |
| | CGGTGGGTTTTCTGGGCGACAAGCATC |
| | GCCGGCGCCGTGATCCAACTAATCGGG |
| | CTGGCCTTCCTGCCCGAGACATATGCA |
| | CCGACATTGCTCAAACGACGCGCGCGG |
| | ACCCTGCGCCAGAAAACTGGCGACGAG |
| | GGATACCACACGGAAGCAGACACGCAG |
| | AACGCAAGCCTGATGTCCACATTGCAG |
| | CGCGCTCTCGTCCGCCCATTCATCCTC |
| | CTCACCACGCAGCCCATTGTGCAAGTC |
| | ATCGCGCTGTACATGGCGTATCTCTTC |
| | GGATTGTTTTACCTGTTGCTCTCGACC |
| | TTCCCTCAAGTCTGGGAGGAGGTCTAC |
| | GGCGAGAGCGTCGGCATTGGCGGGCTG |
| | AACTACATTTCCCTAGGCATTGGATTT |
| | GTGTTGGGAGCGCAGGTGAACGCGCGC |
| | GCCAGCGACCGCATCTACAAACAGCTC |
| | AAGGCCAAGCGGAATAACACCGGACTA |
| | CCCGAGTTCCGTATCCCGGCTATGTTT |
| | GTCGGCTCATTGTTTATCCCGGTCGGG |
| | TTGTTCTGGTATGGATGGAGTGTGCAG |
| | GCACATATCCACTGGATTATGCCCAAT |
| | ATCGGTATTGTGTTGTTTAGTATTGGG |
| | AGCATTATTTGTCTGCAGTGTATGCAG |
| | ACGTACGTGATCGATAGTTATACGCGG |
| | TTTGCGGCGAGTGGGTTGGCTGCTGCG |
| | GTCGTGTTGAGGAGTCTGGCGGGCTTT |
| | AGTTTCCCGTTGTTTGCGCCGTATATG |
| | TATGATAAGCTGCACTATGGCTGGGGG |
| | AATAGTTTGCTTGGATTTTTGAGTATC |
| | GGGATTGGAGTGCCGGCGCCTTGGGTG |
| | TTTTGGTTCTGGGGGGCGAAGTTGAGG |
| | GGGATGTCGAGGTATGCTTCTGGCTAA |
| | (SEQ ID NO: 9) |

### IV. Methods of Use

The present disclosure further provides methods for using the genetically-engineered cells of the present disclosure. The present disclosure provides methods for treating a subject in need thereof by administering one or more genetically-engineered cells of the present disclosure, e.g., a population of genetically-engineered cells of the present disclosure. In certain embodiments, the genetically-engineered cell administered to a subject generates and secretes a therapeutic molecule for treating the subject. Non-limiting examples of therapeutic molecules that can be generated and secreted are disclosed herein in Section II, e.g., the therapeutic molecule can be a peptide, e.g., a toxin peptide, or a small molecule, e.g., tetracycline or a tetracycline analogue.

In certain embodiments, a method of the present disclosure includes administering one or more live and/or intact genetically-engineered cells, e.g., fungal cells, expressing one or more therapeutic molecules. In certain embodiments, a live genetically-engineered cell refers to a cell that has an intact cell membrane and has one or more of the following properties: (1) has the ability to proliferate, (2) is metabolically active and/or (3) actively expresses a therapeutic molecule.

The methods described herein provides a more cost-effective method for administering a therapeutic molecule to a subject in need thereof without requiring the purification of the therapeutic molecule from the genetically-engineered cell prior to administration to the subject. For example, but not by way of limitation, the generation of a therapeutic molecule by a genetically-engineered cell in situ and administration of such a cell can avoid, prevent and/or reduce the degradation of the therapeutic molecule that can occur during the manufacturing, purification and/or storing process. In certain embodiments, administration of a genetically-engineered cell that expresses and/or secretes a therapeutic molecule allows the continuous treatment of the subject with the therapeutic molecule without the need for multiple administrations.

In certain embodiments, a method of the present disclosure includes administering to the subject in need thereof a cell genetically engineered to generate and secrete a therapeutic molecule for treating the subject. In certain embodiments, the genetically-engineered cell is a fungal cell that produces a therapeutic molecule in situ and secretes the therapeutic molecule. The genetically-administered cell can be administered to the subject by any method relevant to disorder and/or condition being treated. For example, but not by way of limitation, the genetically-engineered cell can be administered by parenteral administration, intraocular administration, intraaural administration, intranasal administration, oral administration, rectal administration, vaginal administration or topical administration. In certain embodiments, the genetically-engineered cell is administered topically. In certain embodiments, the genetically-engineered cell is not administered to the digestive system.

In certain embodiments, a method of the present disclosure includes administering to the subject in need thereof a cell genetically engineered to generate and secrete a small molecule that has anti-inflammatory properties for treating the subject. For example, but not by way of limitation, the method can include administering a cell genetically engineered to generate and secrete an analogue of tetracycline, e.g., TAN-1612, or an analogue thereof. In certain embodiments, the genetically-engineered cell that generates and secretes a small molecule that has anti-inflammatory properties, e.g., TAN-1612 or an analogue thereof, can be administered by parenteral administration, intraocular administration, intraaural administration, intranasal administration, oral administration, rectal administration, vaginal administration or topical administration.

In certain embodiments, a method of the present disclosure includes administering to the subject in need thereof a cell genetically engineered to generate and secrete a small molecule that has antibiotic properties for treating the subject. For example, but not by way of limitation, the method can include administering a cell genetically engineered to generate and secrete tetracycline or an analogue thereof. In certain embodiments, the genetically-engineered cell that generates and secretes a small molecule that has antibiotic properties, e.g., tetracycline or an analogue, can be administered by parenteral administration, intraocular administration, intraaural administration, intranasal administration, oral administration, rectal administration, vaginal administration or topical administration.

In certain embodiments, a method of the present disclosure includes administering to the subject in need thereof a cell genetically engineered to generate and secrete a peptide that has anti-fungal, antibiotic and/or anti-microbial properties for treating the subject. For example, but not by way of limitation, the method can include administering a cell genetically engineered to generate and secrete a fungal toxin peptide. In certain embodiments, the fungal toxin peptide can be a K1, K2 or K28 toxin peptide derived from Saccharomyces cerevisiae. In certain embodiments, the genetically-engineered cell that generates and secretes a small molecule that has anti-fungal, antibiotic and/or anti-microbial properties, e.g., a fungal toxin peptide, can be administered by parenteral administration, intraocular administration, intraaural administration, intranasal administration, oral administration, rectal administration, vaginal administration or topical administration. In certain embodiments, a fungal toxin peptide, e.g., a K1, K2 or K28 toxin peptide, can be administered to treat a fungal infection and/or a bacterial infection.

In certain embodiments, the genetically-engineered cells disclosed herein can be administered to treat various conditions. Non-limiting examples of such conditions include gastrointestinal disorders, constipation, irritable bowel syndrome, hemorrhoids, anal fissures, perianal abscesses, anal fistulas, perianal infections, diverticular diseases, colitis, colon polyps, inflammatory conditions, bacterial infections and skin conditions. In certain embodiments, a skin condition that can be treated with the disclosed genetically-engineered cells include acne, fungal nail infections and skin infections.

Non-limiting conditions that can be treated by tetracycline and analogues thereof include intraabdominal infections, endocarditis, brucellosis, Clostridium difficile infections, gram-negative bacterial infections, urinary tract infections, MRSA and respiratory infections, e.g., pneumonia. Additional conditions include rocky mountain spotted fever, typhus fever, typhus group, q fever, rickettsialpox, Mycoplasma pneumoniae, Lymphogranuloma venereum, trachoma, inclusion conjunctivitis, nongonococcal urethritis (Chlamydia tachomatis), Psittacosis (Chlamydia psittaci), relapsing fever (Borrelia recurrentis), chancroid (Haemophilus ducreyi), plague (Yersinia pestis), cholera (Vibro cholerae), Campylobacter fetus, brucellosis (Brucella sp.), Granuloma inguinale caused by Calymmatobacterium granulomatis, Escherichia coli, Enterobacter aerogenes, Shigella species, Acinetobacter species, respiratory tract infections caused by Haemophilus influenzae, respiratory tract and urinary tract infections caused by Klebsiella species, upper respiratory infections caused by Streptococcus pyogenes, Streptococcus pneumoniae and Hemophilus influenzae, lower respiratory tract infections caused by Streptococcus pyogenes, Streptococcus pneumoniae and Mycoplasma pneumoniae, skin and skin structure and soft tissue infections caused by Streptococcus pyogenes, Staphylococcus aureaus, and susceptible isolates of Escherichia coli, Enterococcus faecalis (vancomycin-susceptible isolates), Staphylococcus aureus (methicillin-susceptible and -resistant isolates), Streptococcus agalactiae, Streptococcus anginosus grp. (includes S. anginosus, S. intermedius and S. constellatus), Streptococcus pyogenes, Enterobacter cloacae, Klebsiella pneumoniae and Bacteroides fragilis, urethral, endocervical or rectal infections, Anthrax due to Bacillus anthracis, tuleramia and meningitis. Additional conditions are disclosed in Chopra et al., Microbiology and Molecular Biology Review 65(2):232-260 (2001), the contents of which are herein disclosed in their entirety.

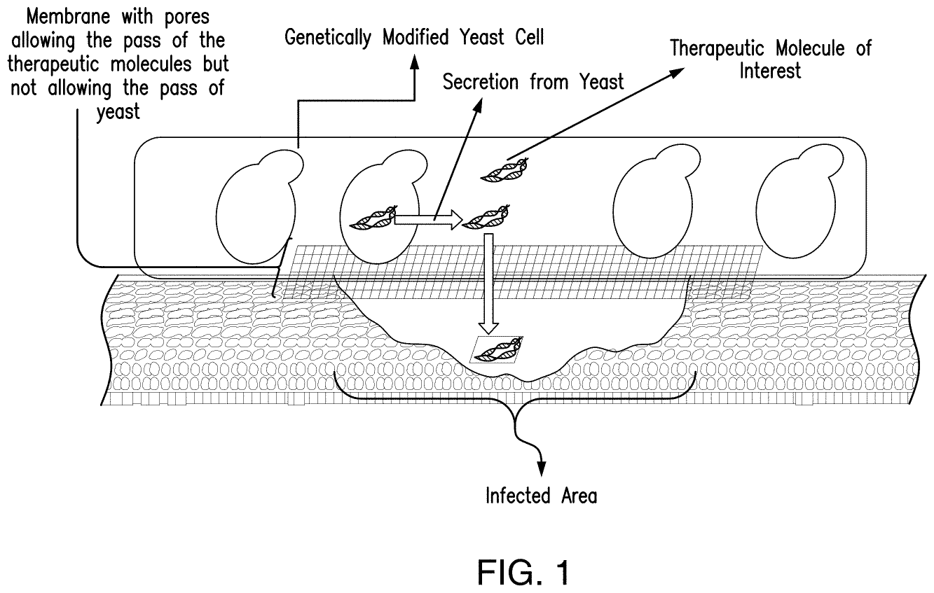

In certain embodiments, a genetically-engineered cell disclosed herein can be topically administered. For example, but not by way of limitation, a genetically-engineered cell disclosed herein can be applied for treatment of skin diseases and/or conditions including skin infections as disclosed above. In certain embodiments, a method of the present disclosure includes administration of a cell genetically engineered to express and/or secrete a small molecule that has anti-inflammatory properties and/or antibiotic properties to treat a subject with a skin condition such as a skin infection. In certain embodiments, a method of the present disclosure includes administration of a cell genetically engineered to express and/or secrete a peptide that has anti-fungal, antibiotic and/or anti-microbial properties to treat a subject with an infection. In certain embodiments, the genetically-engineered cell is applied directly to area that needs to be treated, e.g., directly to the infected area as shown in FIG. 1.

In certain embodiments, a genetically-engineered cell disclosed herein can be administered once a day, twice a day, once a week, twice a week, three times a week, four times a week, five times a week, six times a week, once every two weeks, once a month, twice a month, once every other month or once every third month. In certain embodiments, the genetically-engineered cell can be administered twice a week. In certain embodiments, a genetically-engineered cell disclosed herein be administered once a week. In certain embodiments, a genetically-engineered cell disclosed herein can be administered two times a week for about four weeks and then administered once a week for the remaining duration of the treatment.

### V. Pharmaceutical Compositions

The present disclosure further provides pharmaceutical compositions comprising a genetically-engineered cell for use according to the disclosed methods. In certain embodiments, the pharmaceutical compositions include one or more live and/or intact genetically-engineered cells, e.g., fungal cells, expressing one or more therapeutic molecules.

In certain embodiments, a pharmaceutical composition for use accordingly to the present disclosure can be formulated for parenteral administration, intraocular administration, intraaural administration, intranasal administration, oral administration, rectal administration, vaginal administration or topical administration. In certain embodiments, the pharmaceutical composition is formulated for topical administration.

In certain embodiments, the pharmaceutical composition includes a genetically-engineered cell, disclosed herein, and a pharmaceutically acceptable carrier. “Pharmaceutically acceptable,” as used herein, includes any carrier which does not interfere with the effectiveness of the biological activity of the active ingredients, e.g., the genetically-engineered cell and/or the therapeutic molecule, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioabsorbable matrix materials, implantation elements containing the inhibitor and/or any other suitable vehicle, delivery or dispensing means or material. Such carriers can be formulated by conventional methods and can be administered to the subject.

In certain embodiments, a pharmaceutical composition of the present disclosure can include nutrients for promoting the growth of the one or more genetically-engineered cells present in the composition. For example, but not by way of limitation, a pharmaceutical composition can include vitamins, e.g., peptone, yeast extract, water-soluble vitamins, carbohydrates, e.g., glucose, peptides, amino acids and/or salts. In certain embodiments, the pharmaceutical composition can include growth media for the genetically-engineered cells. In certain embodiments, the growth media is a dry growth media. In certain embodiments, the growth media is a solid form of growth media, e.g., agar-based growth media. Additional non-limiting examples of media and components that can be present in the media to support growth of the genetically-engineered cells are disclosed in Hagerdal et al., Microbial Cell Factories 4:31 (2005), the contents of which are disclosed herein by reference in their entirety.

In certain embodiments, a pharmaceutical composition of the present disclosure can include cofactors of enzymes being expressed by the genetically-engineered cells present in the composition. For example, but not by way of limitation, a pharmaceutical composition can include cofactor F420 or cofactor Fo, which is a functional alternative to F420, for use as cofactors to F420 reductases.

In certain embodiments, the pharmaceutical compositions suitable for use in the present disclosure can include compositions where the genetically-engineered cells are contained in a therapeutically effective amount. A “therapeutically effective amount” refers to an amount of genetically-engineered cells and/or therapeutic molecule produced by the genetically-engineered cells that is able to alleviate one or more symptoms of a condition. The therapeutically effective amount of an active ingredient can vary depending on the active ingredient, e.g., the genetically-engineered cell and/or the therapeutic molecule, formulation used, the condition and its severity, and the age, weight, etc., of the subject to be treated.

In certain embodiments, the pharmaceutical compositions of the present disclosure can be formulated using pharmaceutically acceptable carriers well known in the art that are suitable for parenteral administration, e.g., intravenous administration, intraarterial administration, intrathecal administration, intranasal administration, intramuscular administration, subcutaneous administration and intracisternal administration. For example, but not by way of limitation, the pharmaceutical composition can be formulated as solutions, suspensions or emulsions.

In certain non-limiting embodiments, the pharmaceutical compositions of the present disclosure can be formulated using pharmaceutically acceptable carriers well known in the art that are suitable for intraocular, oral, intranasal or rectal administration. Such carriers enable the pharmaceutical compositions to be formulated as tablets, pills, capsules, liquids, gels, syrups, slurries, suspensions, suppositories and the like, for intraocular, oral, intranasal or rectal administration to the patient to be treated.

In certain embodiments, the tablets, pills, capsules and the like can contain any of the following ingredients, or compounds of a similar nature: a binder such as microcrystalline cellulose, gum tragacanth or gelatin, an excipient such as starch or lactose, a disintegrating agent such as alginic acid, primogel, or corn starch, a lubricant such as magnesium stearate or sterotes, a glidant such as colloidal silicon dioxide, a sweetening agent such as sucrose or saccharin or a flavoring agent.

In certain embodiments, the pharmaceutical compositions can be prepared in the form of suppositories or retention enemas for rectal administration. In certain embodiments, the pharmaceutical compositions can be prepared with carriers that will protect the genetically-engineered cells against rapid elimination from the body, such as a controlled release formulation, including implants. In certain embodiments, biodegradable, biocompatible polymers can be used, such as ethylene vinyl acetate, polyanhydrides, polyglycolic acid, collagen, polyorthoesters and polylactic acid.

In certain embodiments, the pharmaceutical compositions of the present disclosure can be formulated using pharmaceutically acceptable carriers well known in the art that are suitable for topical administration. Such carriers enable the pharmaceutical compositions to be formulated as liquids, gels, creams, syrups, slurries, dispersible powders, suspensions, lotions and the like, for topical administration to the patient to be treated.

In certain embodiments, one or more devices, e.g., inhaler and/or nasal pump, can be used to administer one or more of the disclosed pharmaceutical compositions.

In certain embodiments, a pharmaceutical composition can include one or more lyophilized genetically-engineered cells of the present disclosure.

In certain embodiments, pharmaceutical compositions of the present disclosure can further include one or more additional therapeutics, e.g., a second therapeutic, a third therapeutic or more, for treating a condition of the subject.

### Examples

The following examples are put forth so as to provide those of ordinary skill in the art with a complete disclosure and description of how to make and use the presently disclosed subject matter and are not intended to limit the scope of what the inventors regard as their presently disclosed subject matter. It is understood that various other implementations and embodiments can be practiced, given the general description provided herein.

### Example 1. Using S. cerevisiae for Completing Tetracycline Biosynthesis

The present Example provides for completing tetracycline biosynthesis by using S. cerevisiae. It was found that OxyS in S. cerevisiae performs just one hydroxylation procedure as opposed to two performed in vitro, thus enabling the biosynthesis of tetracycline instead of oxytetracycline. In addition, this Example describes the reduction of the hydroxylation product of OxyS in yeast cell lysate, without the need for heterologous expression of a dedicated reduction enzyme, such as OxyR. Circumventing the need to express OxyR also circumvents the need to heterologously express the biosynthetic pathway of the cofactor or to extraneously supply it or its equivalent. Finally, this Example describes mass spectrometry and UV/Vis results supporting the production of 5a(11a)-dehydrotetracycline using S. cerevisiae.

TABLE 1.2
Strains used in this Example.

| | |
|---|---|
| EH-3-80-3 | FY251 pSP-G1 |
| EH-3-98-6 | FY-251 AL-1-101-C10 |
| EH-3-142-A | AL-1-141-A-C6 |
| EH-3-142-B | AL-1-151-A-C4 |
| EH-3-142-C | AL-1-151-B-C2 |
| EH-5-153-7 | FY-251 AL-173-B-C4 |
| EH-5-153-8 | FY-251 AL-119-A-C7 |
| EH-3-204-5 | FY-251 AL-119-A-C7, AL-215-D-C1 |
| EH-3-204-6 | FY-251 AL-119-A-C7, AL-255-C1 |
| EH-3-204-7 | FY-251 AL-119-A-C7, AL-235-C5 |
| EH-3-204-8 | FY-251 AL-119-A-C7, pRS413-pGAL1 |
| EH-3-204-9 | FY-251 AL-1-101-C10, pRS413-pGAL1 |
| EH-3-248-1 | BJ5464-NpgA AL-1-101-C10 |
| EH-3-248-8 | BJ5464-NpgA pSP-G1 |

OxyS was cloned into pSP-G1 under the transcriptional control of the strong constitutive promoter TEF1 with a FLAG antibody tag at its C-terminus. The resulting plasmid (AL-1-101) was transformed into FY251 and BJ5464-NpgA to generate strains EH-3-98-6 and EH-3-248-1, respectively. The list of plasmids generated in this Example are provided in Table 1.

TABLE 1.3
Plasmids used in this Example.

| | Description |
|---|---|
| pSP-G1 | pTEF1-FLAG-tADH1, pPGK1-MYC-tCYC1, 2μ, Ura |
| pRS413-GAL1 | Shuttle vector (empty), His marker, CEN |
| AL-1-101-C10 | pTEF1-oxyS-tADH1 in pSP-G1 |
| AL-119-A-C7 | pTEF1-oxyS-tADH1, pPGK1-oxyR-tCYC1 in pSP-G1 |
| AL-1-141-A-C6 | pTEF1-oxyS-tADH1, pPGK1-OYEl-tCYC1 in pSP-G1 |
| AL-1-151-A-C4 | pTEF1-oxyS-tADH1, pPGK1-OYE2-tCYC1 in pSP-G1 |
| AL-1-151-B-C2 | pTEF1-oxyS-tADH1, pPGK1-OYE3-tCYC1 in pSP-G1 |
| AL-173-B-C4 | pTEF1-dacO4-tADH1, pPGK1-dacO4-tCYC1 in pSP- |
| | G1 |
| AL-1-183-C-C8 | Fₒ synthase Thermobifida fusca |
| AL-1-183-D-C7 | Fₒ synthase Chlamydomonas reinhardtii |
| AL-215-D-C1 | FGD1 from Mycobacterium tuberculosis |
| AL-255-C1 | F₄₂₀-dependentNADP+ oxidoreductase from |
| | Archeoglobus fulgidus |
| AL-235-C5 | NADPH-dependent F420 reductase from Streptomyces |
| | griseus |

The sequence for the hydroxylase OxyS from Streptomyces rimosus is shown capitalized within the context of the pSP-G1 backbone containing pTEF1, the FLAG tag and tADH1 (partial, uncapitalized) in Table 2. The sequence for the reductase OxyR from Streptomyces rimosus is shown capitalized within the context of the pSP-G1 backbone containing pPGK1, the myc tag and tADH1 (partial, uncapitalized) in Table 2. Sequences for the three F420 reductases from M. tuberculosis, A. fulgidus and S. griseus are shown along with the pGPD (pTDH3) promoter capitalized within the context of the pRS413 backbone containing the tCYC1 terminator (partial, uncapitalized) in Table 2. The NCBI/Genbank reference sequences used for the Fo reductases from M. tuberculosis, A. fulgidus and S. griseus are CP023708.1, NC_000917.1 and NC_010572.1 (6172267.6172977), respectively. Prior to codon optimization, leucine codon in M. tuberculosis F420 reductases in subsequence CATTGAACAACACCCGGTTT (SEQ ID NO: 216) was changed to methionine so that the total sequence matches the protein sequence used for M. tuberculosis F420 crystallization. The sequences for hydroxylases and reductases used in the present Example are shown in Table 2.

TABLE 2
Sequences of Enzymes.

| | |
|---|---|
| pTEF1- | tcaagtttcagtttcatttttcttgttctattacaactttttttacttcttgctcattagaaagaaagcatagcaatct |
| oxyS- | aatctaagttttaattacaagcggccgcATGAGGTACGATGTTGTTATAGCTGGTG |
| tADH1-m- | CAGGACCCACCGGTTTGATGTTAGCATGTGAACTTCGGCTGGCG |
| pSP-G1- | GGTGCCAGAACTTTGGTTTTAGAAAGATTAGCCGAGCCTGTTGA |
| AL-1-101- | CTTCTCGAAAGCTCTAGGAGTCCACGCTCGCACTGTTGAACTAT |
| C10 and | TAGATATGAGAGGCCTCGGTGAAGGATTCCAGGCTGAAGCACC |
| AL-119-A- | AAAGTTAAGAGGTGGTAATTTTGCCTCATTAGGCGTCCCCCTGG |
| C7 | ATTTCTCATCATTTGATACTAGACACCCATATGCATTGTTTGTTC |
| | CACAAGTACGAACTGAAGAACTGCTAACAGGTAGAGCTTTGGA |
| | GCTAGGGGCGGAGCTGCGTCGTGGTCATGCCGTGACCGCCTTGG |
| | AACAAGATGCTGATGGTGTTACTGTGAGCGTGACAGGCCCTGAA |
| | GGCCCATACGAAGTAGAATGTGCTTATTTGGTGGGCTGCGACGG |
| | TGGAGGCAGTACGGTGAGGAAACTATTGGGCATAGATTTTCCAG |
| | GTCAAGACCCACATATGTTTGCTGTCATCGCAGACGCAAGATTC |
| | AGAGAAGAGCTTCCTCACGGAGAAGGTATGGGTCCTATGCGTCC |
| | TTATGGTGTCATGAGACATGACCTTCGTGCATGGTTCGCAGCAT |
| | TTCCGCTAGAACCAGACGTCTACAGAGCAACAGTCGCCTTTTTC |
| | GATAGACCGTATGCTGACAGGAGGGCGCCGGTAACGGAGGAGG |
| | ATGTCAGAGCCGCGTTAACAGAAGTTGCTGGATCTGACTTTGGA |
| | ATGCATGATGTAAGATGGTTATCTCGTTTGACCGATACAAGTAG |
| | ACAAGCAGAAAGGTATAGAGATGGGAGAGTTCTTTTGGCTGGT |
| | GATGCATGCCATATTCATTTGCCCGCTGGTGGCCAGGGACTGAA |
| | CTTAGGATTTCAAGATGCCGTTAACTTGGGTTGGAAGCTTGGTG |
| | CTACCATTGCCGGGACCGCTCCACCAGAGTTATTGGATACGTAT |
| | GAAGCTGAGAGAAGGCCGATAGCCGCCGGTGTTTTGAGAAATA |
| | CAAGGGCTCAAGCTGTTCTAATTGATCCAGATCCTCGCTACGAG |
| | GGTTTAAGGGAATTAATGATTGAATTGTTGCACGTTCCTGAGAC |
| | TAATAGATATTTAGCGGGGCTAATCTCCGCATTAGACGTTAGAT |
| | ACCCAATGGCTGGGGAACATCCATTGCTGGGAAGAAGAGTACC |
| | CGACTTACCTCTTGTCACCGAAGATGGAACAAGACAGTTGTCCA |
| | CTTATTTCCATGCTGCACGTGGCGTATTATTAACGTTAGGTTGTG |
| | ATCAACCACTAGCAGATGAAGCCGCTGCTTGGAAAGATAGGGT |
| | TGATTTAGTTGCAGCTGAAGGTGTGGCGGACCCTGGTTCTGCAG |
| | TAGATGGCTTAACTGCTTTACTTGTAAGGCCTGATGGTTACATTT |
| | GTTGGACAGCTGCCCCAGAAACTGGTACTGATGGATTGACAGAC |
| | GCGCTGCGTACTTGGTTCGGCCCACCTGCAATGgtatcgatggattacaag |
| | gatgacgacgataagatctgagctcttaattaacaattcttcgccagaggtttggtcaagtctccaatcaagg |
| | ttgtcggct (SEQ ID NO: 10). The capitalized sequence is SEQ ID NO: 15. |
| FGD1-in- | ccctcactaaagggaacaaaagctggagctcAGTTTATCATTATCAATACTGCCAT |
| pRS413- | TTCAAAGAATACGTAAATAATTAATAGTAGTGATTTTCCTAACT |
| pGPD- | TTATTTAGTCAAAAAATTAGCCTTTTAATTCTGCTGTAACCCGTA |
| tCYC1-AL- | CATGCCCAAAATAGGGGGCGGGTTACACAGAATATATAACATC |
| 215-D-C1 | GTAGGTGTCTGGGTGAACAGTTTATTCCTGGCATCCACTAAATA |
| | TAATGGAGCCCGCTTTTTAAGCTGGCATCCAGAAAAAAAAAGA |
| | ATCCCAGCACCAAAATATTGTTTTCTTCACCAACCATCAGTTCAT |
| | AGGTCCATTCTCTTAGCGCAACTACAGAGAACAGGGGCACAAA |
| | CAGGCAAAAAACGGGCACAACCTCAATGGAGTGATGCAACCTG |
| | CCTGGAGTAAATGATGACACAAGGCAATTGACCCACGCATGTAT |
| | CTATCTCATTTTCTTACACCTTCTATTACCTTCTGCTCTCTCTGAT |
| | TTGGAAAAAGCTGAAAAAAAAGGTTGAAACCAGTTCCCTGAAA |
| | TTATTCCCCTACTTGACTAATAAGTATATAAAGACGGTAGGTAT |
| | TGATTGTAATTCTGTAAATCTATTTCTTAAACTTCTTAAATTCTA |
| | CTTTTATAGTTAGTCTTTTTTTTAGTTTTAAAACACCAAGAACTT |
| | AGTTTCGACGGATACTAGTAAAATGGCTGAGTTGAAACTTGGCT |
| | ACAAGGCATCAGCTGAACAGTTTGCCCCAAGGGAGTTAGTCGA |
| | ACTAGCAGTCGCTGCCGAAGCTCACGGTATGGATTCCGCTACTG |
| | TTTCCGACCATTTCCAACCATGGAGACACCAAGGTGGCCATGCA |
| | CCATTCTCACTCAGTTGGATGACAGCTGTTGGAGAAAGAACTAA |
| | TAGATTGTTATTGGGGACGTCGGTACTCACCCCGACGTTTAGAT |
| | ACAACCCTGCGGTAATAGCACAGGCCTTTGCTACAATGGGATGT |
| | CTATATCCAAACCGGGTGTTTTTAGGTGTTGGTACTGGAGAGGC |
| | CTTGAATGAAATCGCCACTGGTTATGAAGGTGCTTGGCCTGAGT |
| | TCAAAGAAAGGTTTGCGCGTCTGCGCGAAAGCGTGGGGCTGAT |
| | GAGACAATTATGGTCTGGTGATAGGGTAGATTTTGATGGAGATT |
| | ATTATAGATTAAAAGGCGCGTCTATATATGACGTTCCGGATGGT |
| | GGTGTCCCTGTATATATTGCCGCCGGAGGACCAGCAGTTGCTAA |
| | ATATGCTGGCCGAGCTGGTGACGGTTTCATATGCACTTCAGGCA |
| | AGGGTGAAGAACTTTACACAGAAAAGTTGATGCCCGCCGTCAG |
| | AGAAGGGGCGGCAGCCGCTGACAGGTCTGTTGATGGCATAGAC |
| | AAAATGATTGAGATCAAGATTTCATACGACCCAGATCCCGAATT |
| | AGCAATGAATAACACAAGATTTTGGGCACCTCTTAGTTTGACCG |
| | CAGAACAAAAGCATAGCATCGATGATCCAATTGAAATGGAAAA |
| | AGCTGCTGATGCTTTACCCATCGAGCAAATTGCAAAAAGATGGA |
| | TTGTTGCAAGTGATCCTGATGAAGCAGTGGAGAAAGTAGGACA |
| | GTACGTGACCTGGGGTTTAAATCATCTAGTTTTCCACGCTCCTGG |
| | GCATGATCAAAGAAGATTCTTGGAATTGTTTCAATCTGACCTAG |
| | CGCCAAGACTTCGTCGTCTGGGTTAACTCGAGtcatgtaattagttatgtcacg |
| | cttacattcacgccctccccccacatccgctctaaccgaaaaggaaggagttagacaacctgaagtctagg |
| | tccctatttatttttttatagttatgttagtattaagaacgttatttatatttcaaatttttcttttttttctgtacag |
| | acgcgtgtacgcatgtaacattatactgaaaaccttgcttgagaaggttttgggacgctcgaaggctttaatttgcgg |
| | ccggtacccaat (SEQ ID NO: 11). The capitalized sequence is SEQ ID NO: |
| | 16. |
| FNO-A- | ccctcactaaagggaacaaaagctggagctcAGTTTATCATTATCAATACTGCCAT |
| fulgidus-va- | TTCAAAGAATACGTAAATAATTAATAGTAGTGATTTTCCTAACT |
| pRS413- | TTATTTAGTCAAAAAATTAGCCTTTTAATTCTGCTGTAACCCGTA |
| pGPD- | CATGCCCAAAATAGGGGGCGGGTTACACAGAATATATAACATC |
| tCYC1 | GTAGGTGTCTGGGTGAACAGTTTATTCCTGGCATCCACTAAATA |
| | TAATGGAGCCCGCTTTTTAAGCTGGCATCCAGAAAAAAAAAGA |
| | ATCCCAGCACCAAAATATTGTTTTCTTCACCAACCATCAGTTCAT |
| | AGGTCCATTCTCTTAGCGCAACTACAGAGAACAGGGGCACAAA |
| | CAGGCAAAAAACGGGCACAACCTCAATGGAGTGATGCAACCTG |
| | CCTGGAGTAAATGATGACACAAGGCAATTGACCCACGCATGTAT |
| | CTATCTCATTTTCTTACACCTTCTATTACCTTCTGCTCTCTCTGAT |
| | TTGGAAAAAGCTGAAAAAAAAGGTTGAAACCAGTTCCCTGAAA |
| | TTATTCCCCTACTTGACTAATAAGTATATAAAGACGGTAGGTAT |
| | TGATTGTAATTCTGTAAATCTATTTCTTAAACTTCTTAAATTCTA |
| | CTTTTATAGTTAGTCTTTTTTTTAGTTTTAAAACACCAAGAACTT |
| | AGTTTCGACGGATACTAGTAAAATGAGGGTAGCTTTACTTGGTG |
| | GTACAGGAAATCTTGGAAAAGGCCTTGCCCTACGACTGGCCACG |
| | CTGGGCCATGAAATCGTAGTCGGAAGCAGAAGAGAAGAAAAGG |
| | CAGAGGCTAAAGCAGCAGAATATAGGCGCATTGCAGGTGACGC |
| | TTCCATCACTGGTATGAAAAATGAAGATGCCGCTGAGGCATGCG |
| | ACATTGCTGTCTTGACCATTCCTTGGGAACATGCTATTGACACTG |
| | CCAGAGATTTGAAGAATATTTTACGTGAGAAAATTGTTGTATCA |
| | CCATTAGTTCCAGTGTCAAGAGGTGCAAAGGGCTTCACCTACTC |
| | GTCCGAAAGATCAGCGGCCGAGATTGTGGCTGAAGTTCTGGAAT |
| | CTGAAAAAGTTGTTTCTGCGTTGCACACAATACCTGCTGCAAGA |
| | TTTGCCAACTTGGATGAAAAATTTGATTGGGATGTTCCTGTTTGT |
| | GGTGATGATGACGAAAGTAAGAAAGTCGTCATGTCTTTAATAAG |
| | TGAAATAGATGGGTTGAGGCCGTTAGATGCTGGTCCCCTCTCTA |
| | ACTCCCGTTTAGTGGAGAGCCTAACTCCATTGATATTGAACATC |
| | ATGAGATTCAATGGAATGGGTGAACTAGGGATCAAGTTTTTATA |
| | ACTCGAGtcatgtaattagttatgtcacgcttacattcacgccctccccccacatccgctctaaccgaa |
| | aaggaaggagttagacaacctgaagtctaggtccctatttatttttttatagttatgttagtattaagaacgttatt |
| | tatatttcaaatttttcttttttttctgtacagacgcgtgtacgcatgtaacattatactgaaaaccttgcttgagaa |
| | ggttttgggacgctcgaaggctttaatttgcggccggtacccaat (SEQ ID NO: 12). The |
| | capitalized sequence is SEQ ID NO: 17. |
| FNO-S- | ccctcactaaagggaacaaaagctggagctcAGTTTATCATTATCAATACTGCCAT |
| Griseus-in- | TTCAAAGAATACGTAAATAATTAATAGTAGTGATTTTCCTAACT |
| pRS413- | TTATTTAGTCAAAAAATTAGCCTTTTAATTCTGCTGTAACCCGTA |
| pGPD- | CATGCCCAAAATAGGGGGCGGGTTACACAGAATATATAACATC |
| tCYC1 | GTAGGTGTCTGGGTGAACAGTTTATTCCTGGCATCCACTAAATA |
| | TAATGGAGCCCGCTTTTTAAGCTGGCATCCAGAAAAAAAAAGA |
| | ATCCCAGCACCAAAATATTGTTTTCTTCACCAACCATCAGTTCAT |
| | AGGTCCATTCTCTTAGCGCAACTACAGAGAACAGGGGCACAAA |
| | CAGGCAAAAAACGGGCACAACCTCAATGGAGTGATGCAACCTG |
| | CCTGGAGTAAATGATGACACAAGGCAATTGACCCACGCATGTAT |
| | CTATCTCATTTTCTTACACCTTCTATTACCTTCTGCTCTCTCTGAT |
| | TTGGAAAAAGCTGAAAAAAAAGGTTGAAACCAGTTCCCTGAAA |
| | TTATTCCCCTACTTGACTAATAAGTATATAAAGACGGTAGGTAT |
| | TGATTGTAATTCTGTAAATCTATTTCTTAAACTTCTTAAATTCTA |
| | CTTTTATAGTTAGTCTTTTTTTTAGTTTTAAAACACCAAGAACTT |
| | AGTTTCGACGGATACTAGTAAAATGACTACTCAAGACAGTGGGT |
| | CTGCACCGAAGCCTCCCGCCAAAGATCCATGGGATTTGCCAGAT |
| | GTCAGCGCACTGTCTGTTGGTGTCCTAGGTGGTACAGGACCACA |
| | GGGCAGGGGATTAGCCTACAGATTGGCGCGTGCTGGTCAAAAA |
| | GTGACCTTGGGCTCAAGAGATGCTGGACGCGCTGCTGATGCAGC |
| | GGCAGAGCTGGGCCATGGTGTGGAAGGTACTGATAATGCAGAA |
| | TGCGCAAGAAGATCCGACATCGTTATTGTTGCTGTACCTTGGGA |
| | CGGTCATGCTAAAACTTTGGAAAGTTTAAGAGAAGAGTTGGCTG |
| | GCAAGCTGGTTATCGACTGTGTTAACCCCTTGGGGTTTGATAAG |
| | AAAGGTGCTTATGCTTTAAAACCAGAGGAGGGCTCGGCCGCTG |
| | AACAAGCTGCGGCGCTTCTCCCTGATTCACGAGTAACAGCAGCT |
| | TTCCACCATTTATCTGCTGTGCTTTTACAAGATGAATCCATTGAA |
| | GAAATTGATACCGATGTTTTGGTCTTAGGTGAAGCAAGGGCAGA |
| | CACGGATATTGTCCAGGCACTAGCAGGGCGTATACCAGGTATGA |
| | GAGGAATATTTGCCGGTCGTCTAAGAGGTGCCCACCAAGTTGAA |
| | TCACTTGTAGCCAATTTAATATCTGTAAACAGAAGGTATAAGGC |
| | TCATGCCGGACTAAGGGCCACAGACGTGTAACTCGAGtcatgtaattag |
| | ttatgtcacgcttacattcacgccctccccccacatccgctctaaccgaaaaggaaggagttagacaacctg |
| | aagtctaggtccctatttatttttttatagttatgttagtattaagaacgttatttatatttcaaatttttctttttt |
| | ttctgtacagacgcgtgtacgcatgtaacattatactgaaaaccttgcttgagaaggttttgggacgctcgaaggcttt |
| | aatttgcggccggtacccaat (SEQ ID NO: 13). The capitalized sequence is SEQ |
| | ID NO: 18. |
| pPGK1- | Tggaatggcgggaaagggtttagtaccacatgctatgatgcccactgtgatctccagagcaaagttcgttc |
| oxyR- | gatcgtactgttactctctctctttcaaacagaattgtccgaatcgtgtgacaacaacagcctgttctcacaca |
| tCYC1 in | ctcttttcttctaaccaagggggtggtttagtttagtagaacctcgtgaaacttacatttacatatatataaacttg |
| pSP-G1 | cataaattggtcaatgcaagaaatacatatttggtcttttctaattcgtagtttttcaagttcttagatgctttcttttt |
| AL-119-A- | ctcttttttacagatcatcaaggaagtaattatctactttttacaacaaatataaaacaaggatccATGCCA |
| C7 and | TTCACTCAAAAGGAGATCACGTATTTAAGGGCTCAAGGCTACGG |
| AL-1-26-1- | CCGACTAGCCACCGTCGGTGCTCACGGTGAACCTCACAACGTGC |
| C1 | CGGTATCTTTTGAAATTGATGAAGAAAGAGGTACCATTGAAATA |
| | ACAGGAAGGGATATGGGACGTTCCAGAAAATTCAGAAATGTTG |
| | CCAAAAGTGACAGAGTGGCATTTGTTGTTGATGATGTTCCATGC |
| | AGAGACCCAGAAGTTGTCAGAGCTGTTGTAATACATGGTACTGC |
| | ACAGGCGCTTCCCACAGGCGGAAGAGAAAGGCGTCCTCATTGT |
| | GCTGACGAAATGATTAGAATTCATCCAAGAAGAATAGTAACAT |
| | GGGGTATCGAAGGTGATTTGTCAACTGGTGTTCATGCAAGAGAT |
| | ATTACTGCTGAAGATGGTGGTAGAAGGgtcgacatggaacagaagttgatttcc |
| | gaagaagacctcgagtaagcttggtaccgcggctagctaagatccgctctaaccgaaaaggaaggagtta |
| | gacaacctgaagtctaggtccctatttatttttttatagttatgttagtattaagaacgttatttatatttcaaattttt |
| | cttttttttctgtacagacgcgtgtacgcatgtaacattatactgaaaaccttgcttgagaaggttttgggacgctc |
| | gaagatccagctgcattaatgaatcggccaacgcgcggggagaggcggtttgcgtattgggcgctcttcc |
| | gcttcctcgctcactgactcgctgcgctcgg (SEQ ID NO: 14). The capitalized |
| | sequence is SEQ ID NO: 59. |

1. 6-hydroxylation of anhydrotetracyclines.

The first procedure required to convert anhydrotetracyclines to tetracyclines is the 6-hydroxylation of the anhydrotetracyclines. To test the capacity of Saccharomyces cerevisiae to hydroxylate an anhydrotetracycline such as the model hydroxylating enzyme OxyS was used together with its native substrate, anhydrotetracycline (FIG. 2).

Fresh patches of strains harboring the plasmid for the hydroxylation and/or reduction enzyme and control strains were inoculated in 5 mL selective media (U⁻ or HU⁻) in 15 mL culture tubes (Corning 352059) and placed in shaker overnight to OD600 2-3. Overnight cultures were used to inoculate 100 mL selective media (U⁻ or HU⁻) cultures in 500 mL conical flasks with a starting OD of 0.01-0.05. Cells were grown to final OD of 0.6-0.8 before pelleting in 2-50 mL tubes (Corning 352098) at 4° C., 4,000 rpm for 20 min. Each pellet was redissolved in 0.5 mL H₂O and the suspension was distributed into two pre-sterilized 1.5 mL Eppendorf tubes and pelleted at 14,000 rpm for 10 min at 4° C. Pellets were stored at −20° C. prior to further use.

Pellets were weighed and thawn on ice. A 99:1 mixture of Y-PER yeast protein extraction reagent (ThermoFisher Scientific 78991) and HALT protease inhibitor cocktail (ThermoFisher Scientific PI87786) was added in a ratio of 3 μL mixture per mg pellet and placed on orbital shaker for 20 min at room temperature, followed by 10 min centrifugation at 14,000 rpm at 4° C. and the cell lysate was transferred to a new 1.5 mL Eppendorf tube, kept on ice and used within 1 h.

The cell lysate (80 μL) was added as the last component to a 4 mL vial (Chemglass CG-4900-01) containing 280 μL of 143 mM TRIS (pH 7.45), 7.7 mM anhydrotetracycline HCl (AdipoGen CDX-A0197-M500) and 4.3 mM NADPH tetrasodium hydrate (Sigma-Aldrich N7505), 1.3 μL/mL mercaptoehtanol and 40 μL glucose (278 mM). In the tests indicated as +G6P, glucose-6-phosphate was added as well to a final concentration of 10 mM. A septum was placed on top of the vial through which a needle was inserted to allow air exchange and the reaction was left at room temperature overnight. After this time, 1 mL of MeOH was added, the contents were mixed, and the reaction was filtered through a PTFE 0.2 μm filter (Acrodisc 4423) prior to analysis by mass and UV/VIS spectrometry.

Fresh patches of strains harboring the plasmid for the hydroxylation and/or reduction enzyme and control strains were inoculated in 5 mL selective media (U⁻ or HU⁻) in 15 mL culture tubes (Corning 352059) and placed in shaker overnight to OD600 2-3. Overnight cultures were used to inoculate 100 mL selective media (U⁻ or HU⁻) cultures in 500 mL conical flasks with a starting OD of 0.08. Cells were grown to an OD of 1.3 before placing at 15° C. for an additional 10 h until a final OD of 1.65. Cells were then pelleted in 2 50 mL tubes (Corning 352098) at 10° c., 3,500 rpm for 5 min. Each pellet was redissolved in 0.5 mL H₂O and the suspension was distributed into a pre=sterilized 1.5 mL Eppendorf tube and pelleted at 11,000 rpm for 3 min at 10° c. Pellets were placed on ice and used within 1 h. Pellets from 50 mL culture were redissolved in H₂O (1,025 μL) and added as the last component to 15 mL culture tubes (Corning 352059) containing 1,100 μL of 8 mg/mL anhydrotetracycline HCl, 125 μL glucose solution in H₂O (40%) and 250 μL 1 M TRIS buffer pH 7.45 and were placed in shaker at 350 rpm at 21° C. for 27 h. Cultures were then pelleted and the supernatant was diluted 10× into H₂O before being used for UV/VIS spectral measurements.

As mentioned above, the first procedure to convert anhydrotetracyclines to tetracyclines is the 6-hydroxylation of the anhydrotetracyclines. To test the capacity of Saccharomyces cerevisiae to hydroxylate an anhydrotetracycline, a model hydroxylating enzyme OxyS was used together with its native substrate, anhydrotetracycline (FIG. 2).

In order to facilitate the co-expression of a reductase enzyme, to append antibody tags to both hydroxylase and reductase and to constitutively express both enzymes the expression plasmid pSP-G1 was chosen. OxyS was cloned into pSP-G1 under the transcriptional control of the strong constitutive promoter TEF1 with a FLAG antibody tag at its C-terminus. The resulting plasmid (AL-1-101) was transformed into FY251 and BJ5464-NpgA to generate strains EH-3-98-6 and EH-3-248-1, respectively.

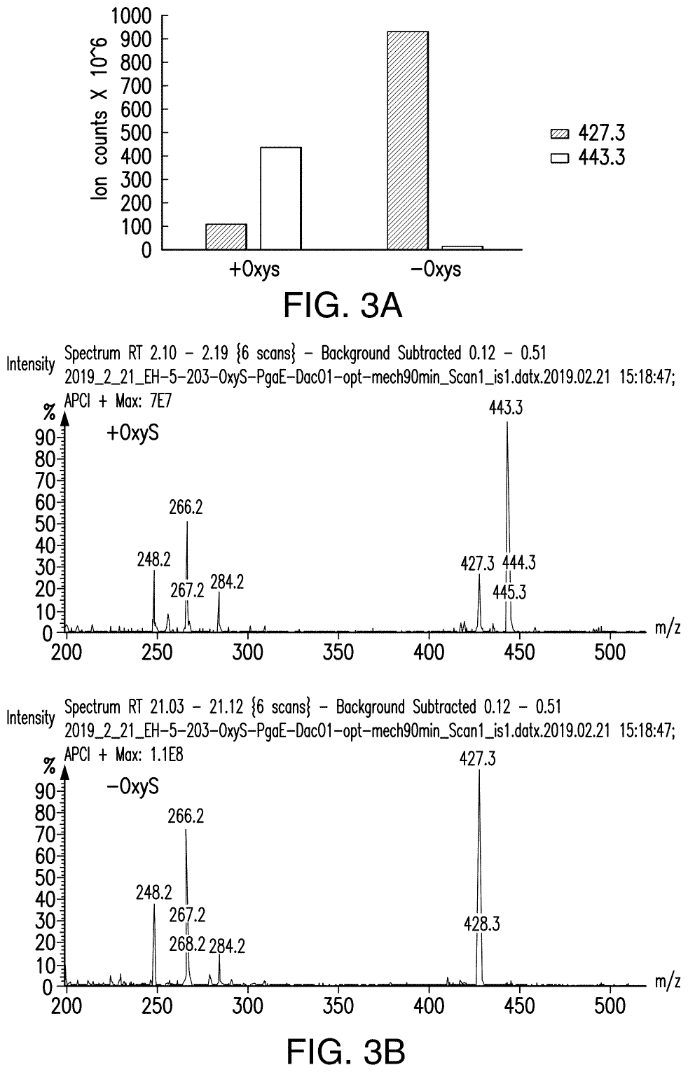

The OxyS-catalyzed reaction to convert anhydrotetracycline to 5a(11a)-dehydrotetracycline in S. cerevisiae was supported by mass spectrometry. When a cell lysate expressing OxyS was added to anhydrotetracycline the molecular ion corresponding to 5a(11a)-dehydrotetracycline ([M+H]⁺) has higher ion counts compared to anhydrotetracycline ([M+H]⁺). However, the opposite was observed when a cell lysate not expressing OxyS is added to anhydrotetracycline (FIG. 3).

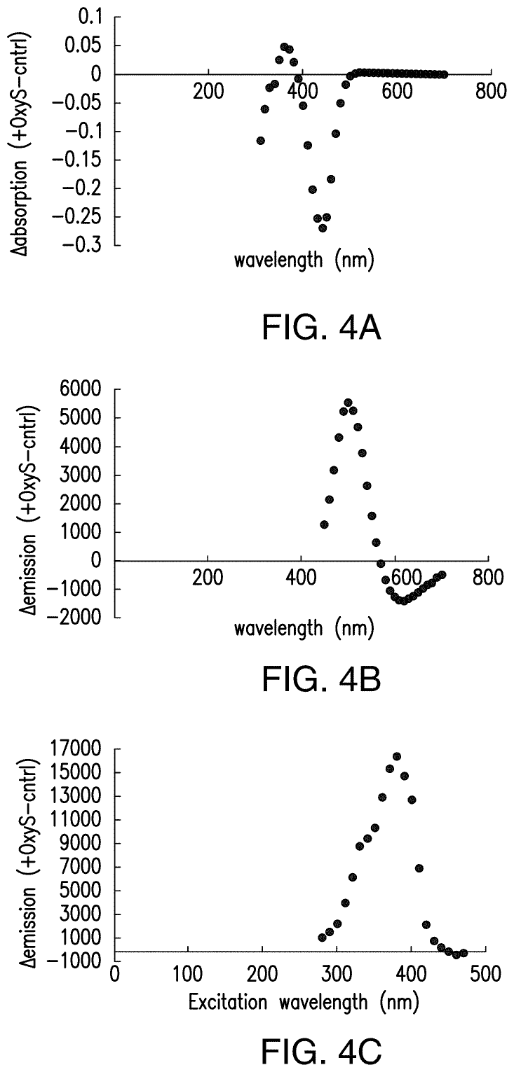

The ability of OxyS to hydroxylate anhydrotetracycline was then tested in whole cells. As for the cell lysate, the molecular ion corresponding to 5a(11a)-dehydrotetracycline had much higher ion counts in the +OxyS sample relative to the −OxyS sample. Given that anhydrotetracycline absorbs and fluoresces in the visible range, it was logical to test whether the reaction can be monitored using a simple UV/Vis assay using a spectrophotometer. Indeed, reducing the −OxyS spectrum from the +OxyS spectrum shows a reduction in the 440 nm absorption and 570 nm emission peaks corresponding to anhydrotetracycline and a formation of 380 nm absorption and 500 nm emission peaks (FIG. 4). The blue shift in absorption and emission is supportive for the formation of 5a(11a)-dehydrotetracycline from anhydrotetracycline as anhydrotetracycline's conjugation in the CD-rings is expected significantly reduced by 6-position hydroxylation (FIG. 2). Indeed, the corresponding red shift in the absorption spectrum for the opposite transformation, where chlortetracycline is converted to anhydrochlortetracycline is noted (λₘₐₓ=373 nm to 438 nm), respectively.

2. Reduction of 5a(11a) Double Bond.

The second procedure in the synthesis of tetracycline from an anhydrotetracycline is to reduce the 5a(11a) double bond (FIG. 2). In order to execute the reduction procedure, OxyR, the reductase of 5a(11a)-dehydrooxytetracycline, was placed under the control of a PGK1 promoter in the pSP-G1-OxyS plasmid AL-1-101 to generate AL-119-A-C7.

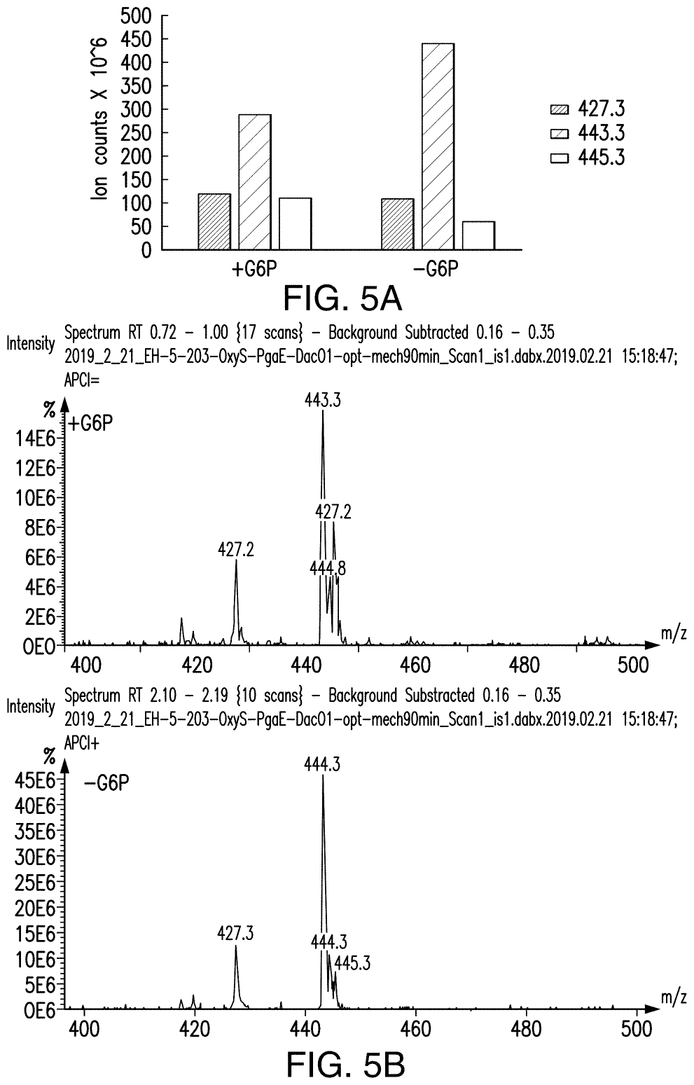

Synthetic Fo was exogenously added to a yeast strain expressing OxyS, OxyR and an Fo reductase. Fo reductase was placed under the control of pGPD (pTDH3) on pRS413 to generate AL-215-D-C1, AL-255-C1 and AL-235-05, encoding F420 reductase from Mycobacterium tuberculosis, Archeoglobus fulgidus and Streptomyces griseus, respectively. The reaction mixture in TRIS buffer (pH 7.45) also contained anhydrotetracycline, glucose NADPH, and in the case of Fo reductase from M. tuberculosis, glucose-6-phosphate for Fo reduction. It was found that the levels of the molecular ion peak corresponding to tetracycline, the reduction product of 5a(11a)-dehydrotetracycline, were increased when glucose-6-phosphate was added (FIG. 5). Contrary to the expectation that glucose-6-phosphate serves as a substrate for the Fo reductase, it was found that neither Fo, the Fo reductase or OxyR increased the levels of the molecular ion peak corresponding to tetracycline either in the presence or absence of glucose-6-phosphate.

The data presented in this Example supports that the use of OxyS as the sole heterologously expressed enzyme in S. cerevisiae allows both a single hydroxylation and reduction procedures to take place using S. cerevisiae. This result is unexpected for two reasons: one, OxyS is known to perform two hydroxylation procedures in vitro and in vivo. Two, it was expected that the co-expression of the dedicated reductase enzyme and the supply/heterologous biosynthesis of its nonnative cofactor can be required for the reduction procedure.

Similar chemistry for completing tetracycline biosynthesis for additional tetracyclines including but limited to oxytetracycline, chlortetracycline, dactylocycline, and their analogs could also be pursued with combinations of the following enzymes: CtcN, SsfO1, DacO1, CtcR, DacO4 and their homologs.

The hydroxylation and reduction processes can be combined with the rest of tetracycline biosynthetic pathways for a complete biosynthesis of tetracyclines in yeast. The additionally required enzymes include but are not limited to DacA, DacB, DacC, DacD, DacG, DacH, DacK, DacM1, DacM2, DacM3, DacN, DacO2, DacO3, DacO5, DacJ, DacP, DacQ, DacE, DacT1, DacT2, DacT3, DacR1, DacR2, DacR3, DacS1, DacS2, DacS3, DacS4, DacS5, DacS6, DacS7, DacS8, DacS9, DacP1, DacP2, DacP3, OxyA, OxyB, OxyC, OxyD, OxyG, OxyF, OxyH, OxyI, OxyK, OxyL, OxyM4, OxyN, OxyP, OxyQ, TA1, OtcG, OtrA, OtrB, ctc9, ctc8, ctc7, ctc6, ctc5, ctc4, ctc3, ctcA, ctcB, ctcC, ctcD, ctcE, ctcF, ctcG, ctcH, ctI, ctcJ, ctcK, ctcL, ctcM, ctcO, ctcP, ctcQ, ctcS, ctcT, ctcU, ctcV, ctcW, ctcX, ctcY, ctcZ, ctc1, ctc2, ctc10, vrtA, vrtB, vrtC, vrtD, vrtE, vrtF, vrtG, vrtG, vrtI, vrtJ, vrtK, vrtL, vrtR1, vrtR2, SsfA, SsfB, SsfC, SsfD, SsfY1, SsfY2, SsfY4, SsfL2, SsfM4, SsfO2, SsfV, SsfT1, SsfT2, SsfR, SsfS1 and SsfS3.

### Example 2. Biosynthesis of 6-Demethyl-6-Epitetracyclines Using Saccharomyces cerevisiae

The present Example provides for biosynthesis of 6-demethyl-6-epitetracyclines using Saccharomyces cerevisiae. As opposed to the previous Example, where 6β-hydroxylation of an anhydrotetracycline is required, synthesis of 6-demethyl-6-epitetracyclines requires 6α-hydroxylation by an enzyme such as, for example, DacO1.

The first two procedures in the synthesis of 6-demethyl-6-epiglycotetracyclines result in the synthesis of 6-demethyl-6-epitetracyclines from anhydrotetracyclines. This example shows the progress towards developing an S. cerevisiae platform for 6-demethyl-6-epitetracyclines biosynthesis using bacterial hydroxylase enzymes as a starting point for directed evolution and rational design as well as anhydrotetracycline as a model substrate. DacO1, a bacterial flavin-dependent monooxygenase homologous to OxyS was supposed to perform a 6α-hydroxylation on its native substrate that leads to 6-epiglycotetracyclines in its native host, Dactylosporangium sp. SC 14051 (ATCC 53693). Thus, DacO1, along with other bacterial hydroxylases, is used here as a template to evolve a 6α-hydroxylase in S. cerevisiae towards the biosynthesis of 6-demethyl-6-epiglycotetracyclines.

Yeast strains tested in the present Example are provided in Table 3. Plasmids used in this Example are provided in Table 4.

TABLE 3
Yeast Strains.

| | Genotype |
|---|---|
| FY251 | MATa leu2Δ1 trpΔ63 ura3-52 his3-200 |
| BJ5464- | MATa ura3-52 his3-Δ200 leu2-Δ1 trp1 pep4::HIS3 |
| NpgA | δ:: pADH2-npgA-tADH2prb1Δ1.6R can1 GAL |
| EH-3-80-2 | FY251 AL-1-31-C7 |
| EH-3-80-3 | FY251 pSP-G1 |
| EH-3-98-6 | FY-251 AL-1-101-C10 |
| EH-5-153-7 | FY-251 AL-173-B-C4 |
| EH-5-153-8 | FY-251 AL-119-A-C7 |
| EH-3-248-1 | BJ5464-NpgA AL-1 -101 -C10 |
| EH-3-248-2 | BJ5464-NpgA AL-1-119-A-C7 |
| EH-3-248-3 | BJ5464-NpgA AL-1-26-1-C1 |
| EH-3-248-4 | BJ5464-NpgA AL-1-31-C7 |
| EH-3-248-5 | BJ5464-NpgA AL-1-234-B-C2 |
| EH-3-248-6 | BJ5464-NpgA AL-1-239-B-C3 |
| EH-3-248-7 | BJ5464-NpgA AL-1-234-C-C1 |
| EH-3-248-8 | BJ5464-NpgA pSP-G1 |
| EH-3-270-9 | FY-251 AL-2-8-D |
| EH-5-19-2 | FY-251 AL-2-8-D |
| EH-5-98-1 | FY251 EH-5-49-A |
| EH-5-98-2 | FY251 EH-5-49-B |
| EH-5-98-3 | FY251 EH-5-49-C |
| EH-5-98-4 | FY251 EH-5-80-1 |
| EH-5-98-5 | FY251 EH-5-80-2 |
| EH-5-98-6 | FY251 EH-5-80-3 |
| EH-5-98-7 | FY251 EH-5-80-4 |
| EH-5-98-8 | FY251 EH-5-80-5 |
| EH-5-98-9 | FY251 EH-5-80-6 |
| EH-5-98-10 | FY251 EH-5-80-7 |
| EH-5-98-11 | FY251 EH-5-80-8 |
| EH-5-98-12 | FY251 EH-5-80-9 |
| EH-5-98-13 | FY251 EH-5-80-10 |
| EH-5-98-14 | FY251 EH-5-80-11 |
| EH-5-98-15 | FY251 EH-5-80-12 |
| EH-5-98-16 | FY251 AL-1-239-A |
| EH-5-98-17 | FY251 AL-1-239-B |
| EH-5-98-18 | FY251 EH-5-90-A |
| EH-5-98-19 | FY251 EH-5-90-A |
| EH-5-115-1 | FY251 EH-5-90-A EH-5-105-A |
| EH-5-115-2 | FY251 AL-1-31-C7 EH-5-105-A |
| EH-5-115-3 | FY251 pSP-G1 EH-5-105-A |
| EH-5-115-4 | BJ5464-NpgA AL-1-226-A |
| EH-5-115-5 | BJ5464-NpgA AL-1-239-A |
| EH-5-163-1 | BJ5464-NpgA EH-5-49-A |
| EH-5-163-2 | BJ5464-NpgA EH-5-49-C |
| EH-5-163-3 | BJ5464-NpgA EH-5-80-6 |
| EH-5-163-4 | BJ5464-NpgA EH-5-80-7 |
| EH-5-163-5 | BJ5464-NpgA EH-5-80-9 |
| EH-5-163-6 | BJ5464-NpgA EH-5-80-10 |
| EH-5-163-7 | BJ5464-NpgA EH-5-80-11 |
| EH-5-163-8 | FY251 AL-1-234-C-C1 |

TABLE 4
Plasmids.

| | Description |
|---|---|
| pSP-G1 | pTEF1-FLAG-tADH1, pPGK1-MYC-tCYC1, 2μ, Ura |
| pRS413-pGAL1 | Shuttle vector (empty), His marker, CEN |
| pRS416-pGAL1 | Shuttle vector (empty), Ura marker, CEN |
| pRS426-pGAL1 | Shuttle vector (empty), Ura marker, 2μ |
| AL-1-26-1-C1 | pPGK1-oxyR-tCYC1 inpSP-G1 |
| AL-1-31-C7 | pTEF1-dacO1-tADH1 inpSP-G1 |
| AL-1-101-C10 | pTEF1-myS-tADH1 inpSP-G1 |
| AL-119-A-C7 | pTEF1-oxyS-tADH1, pPGK1-oxyR-tCYC1 in pSP-G1 |
| AL-173-B-C4 | pTEF1-dacO1-tADH1, pPGK1-dacO4-tCYC1 in pSP- |
| | G1 |
| AL-1-226-A-C2 | pTEF1-oxyS-tADH1 in pSP-G1 JCAT codon opt. |
| AL-1-234-B-C2 | pTEF1-ctcN-tADH1 inpSP-G1 JCAT codon opt. |
| AL-1-234-C-C1 | pTEF1-pgaE-tADH1 in pSP-G1 JCAT codon opt. |
| AL-1-239-A-C2 | pTEF1-dacO1-tADH1 in pSP-G1 JCAT codon opt. |
| AL-1-239-B-C3 | pTEF1-ssfO1-tADH1 in pSP-G1 JCAT codon opt. |
| AL-2-8-D | Error-prone mutagenesis library of AL-1-31-C7 |
| EH-5-49-A | pTEF1-UBI4-dacO1-tADH1 inpSP-G1 |
| EH-5-49-B | pTEF1-SOD1-dacO1-tADH1 inpSP-G1 |
| EH-5-49-C | pTEF1-GAL1-dacO1-tADH1 in pSP-G1 |
| EH-5-80-1 | pGAL1-dacO1-tCYC1 inpRS416 |
| EH-5-80-2 | pGAL1-dacO1-tCYC1 in pRS426 |
| EH-5-80-3 | pTEF1-dacO1-tADH1 inpSP-Gl (no antibody tag) |
| EH-5-80-4 | pADH2-dacO1-tCYC1 inpRS416 |
| EH-5-80-5 | pADH2-dacO1-tCYC1 in pRS426 |
| EH-5-80-6 | pTEF1-UBI4-IFNG-dacO1-tADH1 in pSP-G1^{b} |
| EH-5-80-7 | pTEF1-IFNG-dacO1-tADH1 inpSP-G1^{b} |
| EH-5-80-8 | pPGK1-dacO4-dacO1-tCYC1 in pSP-G1 |
| EH-5-80-9 | pPGK1-dacO1-dacO4-tCYC1 in pSP-G1 |
| EH-5-80-10 | pTEF1-oxyS-dacO1-37-tADH1 in pSP-G1 |
| EH-5-80-11 | pTEF1-oxyS-dacO1-87-tADH1 in pSP-G1 |
| EH-5-80-12 | pTEF1-oxyS-dacO1-191-tADH1 in pSP-G1 |
| EH-5-90-A | pTEF1-dacO1-tADH1, pPGK1-dacJ-tCYC1 in pSP-G1 |
| EH-5-105-A | pGPD-dacM2-tCYC1 inpRS413 |

Sequences for the hydroxylases OxyS, DacO1, PgaE, SsfO1, CtcN and the DacO1 fusion proteins are shown capitalized within the context of the pSP-G1 backbone containing pTEF1, the FLAG tag and tADH1 (partial, uncapitalized). Sequences for DacO4, the DacO1-DacO4 and DacO4-DacO1 fusions and for DacJ are shown capitalized within the context of the pSP-G1 backbone containing pPGK1, the myc tag and tADH1 (partial, uncapitalized). The sequence for DacM2 is shown along with the pGPD (pTDH3) promoter capitalized within the context of the pRS413 backbone containing the tCYC1 terminator (partial, uncapitalized). The sequences for DacO1 in pRS backbone under the control of pGAL1 or pADH2 is shown capitalized along with the promoter (uncapitalized in the case of pGAL1 and capitalized in the case of pADH2) within the context of the pRS backbone containing the tCYC1 terminator (partial, uncapitalized). Sequences used in the present Example are provided in Table 5.

TABLE 5
Nucleotide Sequences.

| | |
|---|---|
| pTEFl-dacO1-tADH1- | tcaagtttcagtttcatttttcttgttctattacaactttttttacttcttgctcattagaaagaaagcatagcaat |
| in-pSP-G1-AL-l-31-C7 | ctaatctaagttttaattacaagcggccgcATGTTAGTAGCCGGTGGTGGACCA |
| and AL-173-B-C4 | ACAGGGTTATGGTTGGCTGGTGAACTAAGGTTGGCCGGGGTT |
| | AGACCTCTTGTTTTAGAGCGTAGGACAGAACCCTTTCCACAT |
| | AGCAAAGCACTGGGTATCCATCCAAGAACTATTGAAATGTTG |
| | GAGTTGAGAGGCCTTGCTGGTAGATTTTTAGATGGCGCCCCG |
| | AAGCTGCCGAAAGGTCACTTTGCATCGCTTCCTGTGCCCTTA |
| | GACTATGGTGCAATGGATACTCGACATCCGTACGGTGTCTAC |
| | CGTAAACAAGTGGAAACGGAAGCTTTATTAGCCGGCCACGC |
| | AACCAGATTAGGAGTTCCAGTCCGCAGAGGTCATGAACTTGT |
| | AGGCCTAAGACAGCACGATGATGCTGTAGTCGGTACTGTTCA |
| | AGGTCCTCAAGGTAGATACGAAGTCAGAGCCGCTTATCTTGT |
| | TGGTTGTGATGGAGGAGGTTCTACCACAAGACGCCTGGCTGG |
| | GATAGATTTCCCTGGACAAGATCCTGGTGTTGCCGTTTTGAT |
| | GGCTGACGCAAGATTCCGGGACCCATTACCCTCTGATCCAGC |
| | TATGGGTCCATTCAGACGCTATGGAGTCCTGAGACCTGATTT |
| | AAGGGCATGGTTTAGTGCTATTCCTTTACCAGAAGAACAGGG |
| | TTTATGCAGAGTTATGGTTTTTTGGTATGGACGACAATTTGA |
| | AGATAGACGTGCTCCAGTAACTGAGGATGAAATGAGAGCAG |
| | CACTTGTTGATCTAGCGGGAACTGACTTTGGTATGTACGATG |
| | TTCAATGGTTGACCAGATTCACAGACGCTTCCAGGCAAGCTG |
| | CAAGGTATAGATCTGGAAGGGTTTTCTTAGCTGGAGATGCTG |
| | CTCATACTGTCTTCCCCACTGGTGGCCCAGGTTTGAATGCAGG |
| | TTTACAAGATGCCATGAATTTAGGTTGGAAGTTAGCTGGAGC |
| | TGTTCATGGCTGGGGTGGTGCTTTACTAGATTCATATCATGAT |
| | GAGCGTTACCCTGTAGCTGAACAAGTTTTGCGTGACACAAGA |
| | ACACAATGTCTATTGTTAGATCCAGACCCTCGCTTTGTTCCAT |
| | TAAGAGAGACGTTAGCAGAACTATTGAGACTAGAACCCGTAA |
| | ACAGATATTTCACCGGACACAACACGGCTCTAGCAGTTAGAT |
| | ACGATTTGGGTGGTGCCCATCCTGCAACGGGAGGCAGAATGC |
| | CAGATGTCTTGGTCGCAACTGGTGCTGGTCAAAGAAGGTTTT |
| | CAGATTGTTGTGTTACCGGGAGAGGGGTTCTTTTAGTCACTTC |
| | TGGTGCAGATAGGGGGGAACTGTTACGAGGATGGAAAGATA |
| | GAGTGGATGTGGTAACAGGTACCGTGGCAGGACCTCTTGATG |
| | CAGCTGATCATTTAGTTAGACCTGATGGTTATGTTGCATGGGC |
| | CGGTGGAGCTGGACACGATGAACCTGGTTTAGAAACTGCTCT |
| | TAGATTATGGTTTGGTGAACCAGTTAGTgtatcgatggattacaaggatgac |
| | gacgataaAatctgagctcttaattaacaattcttcgccagaggtttggtcaagtctccaatcaaggttgt |
| | cggct (SEQ ID NO: 19). The capitalized sequence is SEQ ID NO: 41. |
| pPGK1-dacO4-tCYC1- | Tggaatggcgggaaagggtttagtaccacatgctatgatgcccactgtgatctccagagcaaagttc |
| AL-173-B-C4 | gttcgatcgtactgttactctctctctttcaaacagaattgtccgaatcgtgtgacaacaacagcctgttct |
| | cacacactcttttcttctaaccaagggggtggtttagtttagtagaacctcgtgaaacttacatttacatat |
| | atataaacttgcataaattggtcaatgcaagaaatacatatttggtcttttctaattcgtagtttttcaagttctt |
| | agatgctttctttttctcttttttacagatcatcaaggaagtaattatctactttttacaacaaatataaaacaag |
| | gatccATGTCATTCACTGCCAAGGAAGTTCAATATTTGCGTTCTC |
| | AGCAATTAGGAAGATTAGCCACCGTTGGTGCTGATGGTACAC |
| | CACATAATGTTCCAGTAGGCTACAGATACAACGCTGACTTGG |
| | GGACCATTGACATCACAGGAAGGGACTTAAGAAGAAGTAGG |
| | AAATATAGAGATGTTCAGGCTGGCTCGCGGGTGGCATTTATT |
| | GTTGATGATCTACCGAGCACAGCACCTATAGTCGCCCGCGGG |
| | CTGGAGGTGAGGGGAACAGCTGAGGCGCTTTCTGGTGAAGA |
| | AGATTTGATACGTGTACGACCTGCAAGAATCGTCACTTGGGG |
| | TATTGAAGCAGATTGGCAAGCTGGTCCTACTGGTAGAACGGT |
| | AAGGCCCACCACTCCAAGATCCGCGACGgtcgacatggaacagaagttga |
| | tttccgaagaagacctcgagtaagcttggtaccgcggctagctaagatccgctctaaccgaaaaggaa |
| | ggagttagacaacctgaagtctaggtccctatttatttttttatagttatgttagtattaagaacgttatttatat |
| | ttcaaatttttcttttttttctgtacagacgcgtgtacgcatgtaacattatactgaaaaccttgcttgagaagg |
| | ttttgggacgctcgaagatccagctgcattaatgaatcggccaacgcgcggggagaggcggtttgcgt |
| | attgggcgctcttccgcttcctcgctcactgactcgctgcgctcgg (SEQ ID NO: 20). The |
| | capitalized sequence is SEQ ID NO: 42. |
| pTEF1-oxyS-tADH1 in | tcaagtttcagtttcatttttcttgttctattacaactttttttacttcttgctcattagaaagaaagcatagcaat |
| pSP-G1 JCAT codon | ctaatctaagttttaattacaagcggccgcATGAGATACGACGTTGTTATCGCT |
| opt. (AL-1-226-A) | GGTGCTGGTCCAACTGGTTTGATGTTGGCTTGTGAATTGAGA |
| | TTGGCTGGTGCTAGAACTTTGGTTTTGGAAAGATTGGCTGAA |
| | CCAGTTGACTTCTCTAAGGCTTTGGGTGTTCACGCTAGAACT |
| | GTTGAATTGTTGGACATGAGAGGTTTGGGTGAAGGTTTCCAA |
| | GCTGAAGCTCCAAAGTTGAGAGGTGGTAACTTCGCTTCTTTG |
| | GGTGTTCCATTGGACTTCTCTTCTTTCGACACTAGACACCCAT |
| | ACGCTTTGTTCGTTCCACAAGTTAGAACTGAAGAATTGTTGA |
| | CTGGTAGAGCTTTGGAATTGGGTGCTGAATTGAGAAGAGGTC |
| | ACGCTGTTACTGCTTTGGAACAAGACGCTGACGGTGTTACTG |
| | TTTCTGTTACTGGTCCAGAAGGTCCATACGAAGTTGAATGTG |
| | CTTACTTGGTTGGTTGTGACGGTGGTGGTTCTACTGTTAGAA |
| | AGTTGTTGGGTATCGACTTCCCAGGTCAAGACCCACACATGT |
| | TCGCTGTTATCGCTGACGCTAGATTCAGAGAAGAATTGCCAC |
| | ACGGTGAAGGTATGGGTCCAATGAGGCCATACGGTGTTATG |
| | AGACACGACTTGAGAGCTTGGTTCGCTGCTTTCCCATTGGAA |
| | CCAGACGTTTACAGAGCTACTGTTGCTTTCTTCGACAGACCA |
| | TACGCTGACAGAAGAGCGCCAGTTACTGAAGAAGATGTTAG |
| | AGCTGCTTTGACTGAAGTTGCTGGTTCTGACTTCGGTATGCA |
| | CGACGTTAGATGGTTGTCTCGTTTGACTGACACTTCTCGTCAA |
| | GCTGAAAGATACAGAGATGGTAGAGTTTTGTTGGCTGGTGAC |
| | GCTTGTCACATCCACTTGCCAGCTGGTGGTCAAGGTTTGAAC |
| | TTGGGTTTCCAAGACGCTGTTAACTTGGGTTGGAAGTTGGGT |
| | GCTACTATCGCTGGTACTGCTCCACCAGAATTGTTGGACACT |
| | TACGAAGCTGAAAGAAGGCCAATCGCTGCTGGTGTTTTGAGA |
| | AACACTAGAGCGCAAGCTGTTTTGATCGACCCAGACCCAAG |
| | ATACGAAGGTTTGAGAGAATTGATGATCGAATTGTTGCACGT |
| | TCCAGAAACTAACAGATACTTGGCTGGTTTGATCTCTGCTTT |
| | GGACGTTAGATACCCAATGGCTGGTGAACACCCATTGTTGGG |
| | TAGAAGAGTTCCAGACTTGCCATTGGTTACTGAAGATGGTAC |
| | TAGACAATTGTCTACTTACTTCCACGCTGCTAGAGGTGTTTTG |
| | TTGACTTTGGGTTGTGACCAACCATTGGCTGACGAAGCTGCT |
| | GCTTGGAAGGACAGAGTTGACTTGGTTGCTGCTGAAGGTGTT |
| | GCTGACCCAGGTTCTGCTGTTGACGGATTAACTGCTTTGTTG |
| | GTTAGACCAGACGGTTACATCTGTTGGACTGCTGCTCCAGAA |
| | ACTGGTACTGACGGTTTGACTGACGCTTTGAGAACTTGGTTC |
| | GGTCCACCTGCTATGgtatcgatggattacaaggatgacgacgataagatctgagctctt |
| | aattaacaattcttcgccagaggtttggtcaagtctccaatcaaggttgtcggct (SEQ ID NO: |
| | 21). The capitalized sequence is SEQ ID NO: 43. |
| pTEF1-dacO1-tADH1 in | tcaagtttcagtttcatttttcttgttctattacaactttttttacttcttgctcattagaaagaaagcatagcaa |
| pSP-G1 JCAT codon | tctaatctaagttttaattacaagcggccgcATGTTGGTTGCTGGTGGTGGTCCAAC |
| opt. (AL-1-239A) | TGGTTTGTGGTTGGCTGGTGAATTGAGATTGGCTGGTGTTAGA |
| | CCATTGGTTTTGGAAAGAAGAACTGAACCATTCCCACACTCTA |
| | AGGCTTTGGGTATCCACCCAAGAACTATCGAAATGTTGGAAT |
| | TGAGAGGTTTGGCTGGTAGATTCTTGGACGGTGCTCCAAAGTT |
| | GCCAAAGGGTCACTTCGCTTCTTTGCCAGTTCCATTGGACTAC |
| | GGTGCTATGGACACTAGACACCCATACGGTGTTTACAGAAAG |
| | CAAGTTGAAACTGAAGCTTTGTTGGCTGGTCACGCTACTAGAT |
| | TGGGTGTTCCAGTTAGAAGAGGTCACGAATTGGTTGGTTTGA |
| | GACAACACGACGACGCTGTTGTTGGTACTGTTCAAGGTCCAC |
| | AAGGTAGATACGAAGTTAGAGCTGCTTACTTGGTTGGTTGTG |
| | ACGGTGGTGGTTCTACTACTAGAAGATTGGCTGGTATCGACTT |
| | CCCAGGTCAAGACCCAGGTGTTGCTGTTTTGATGGCTGACGCT |
| | AGATTCAGAGATCCATTGCCATCTGACCCAGCTATGGGTCCAT |
| | TCAGAAGATACGGTGTTTTAAGACCAGACTTGAGAGCTTGGT |
| | TCTCTGCTATCCCATTGCCAGAAGAACAAGGTTTGTGTAGAGT |
| | TATGGTTTTCTGGTACGGTAGACAATTCGAAGATAGAAGAGC |
| | GCCAGTTACTGAAGATGAAATGAGAGCTGCTTTGGTTGACTT |
| | GGCTGGTACTGACTTCGGTATGTACGACGTTCAATGGTTGACT |
| | AGATTCACTGACGCTTCTCGTCAAGCTGCTAGATACAGATCGG |
| | GTAGAGTTTTCTTGGCTGGTGACGCTGCTCACACTGTTTTCCC |
| | AACTGGTGGTCCAGGTTTGAACGCTGGTTTGCAAGACGCTAT |
| | GAACTTGGGTTGGAAGTTGGCTGGTGCTGTTCACGGTTGGGGT |
| | GGTGCTTTGTTGGACTCTTACCACGACGAAAGATACCCAGTTG |
| | CTGAACAAGTTTTGAGAGACACTAGAACTCAATGTTTGTTGTT |
| | GGACCCAGACCCAAGATTCGTTCCATTGAGAGAAACTTTGGC |
| | TGAATTGTTGAGATTGGAACCAGTTAACAGATACTTCACTGGT |
| | CACAACACTGCTTTGGCTGTTAGATACGACTTGGGTGGTGCTC |
| | ACCCAGCTACTGGTGGTAGAATGCCAGACGTTTTGGTTGCTAC |
| | TGGTGCTGGTCAAAGAAGATTCTCTGACTGTTGTGTTACTGGT |
| | AGAGGTGTTTTGTTGGTTACTTCTGGTGCTGACAGAGGTGAAT |
| | TGTTGAGAGGTTGGAAGGACAGAGTTGACGTTGTTACTGGTA |
| | CTGTTGCTGGTCCATTGGACGCTGCTGACCACTTGGTTAGACC |
| | AGACGGTTACGTTGCTTGGGCTGGTGGTGCTGGTCACGACGA |
| | ACCAGGTTTGGAAACTGCTTTGAGATTGTGGTTCGGTGAACCA |
| | GTTTCTgtatcgatggattacaaggatgacgacgataagatctgagctcttaattaacaattcttcgc |
| | cagaggtttggtcaagtctccaatcaaggttgtcggct (SEQ ID NO: 22). The |
| | capitalized sequence is SEQ ID NO: 44. |
| pTEF1-ctcN-tADH1 in | tcaagtttcagtttcatttttcttgttctattacaactttttttacttcttgctcattagaaagaaagcatagcaa |
| pSP-G1 JCAT codon | tctaatctaagttttaattacaagcggccgcATGGTTGTTGCTGGTGCTGGTCCA |
| opt. (AL-1-234-B-C2) | ACTGGTTTGATGTTGGCTTGTGAATTGGCTTTGGGTGGTGCTA |
| | GAGCTGTTGTTGTTGAAAGAAGAAGAGAACCAGAAAAGCAC |
| | TCTAAGGCTATGGGTATGCAAGGTAGAACTGTTGAATTGTTG |
| | GAATTGAGAGGTTTGTTGGACAGATTCAAGGAAGGTGCTGGT |
| | GTTTTGCAAGGTGGTAACTTCGCTTCTTTGGGTGTTCCAATGA |
| | GATTCGAAGAATTTGACACTCAACCAACTCAATACGTTTTGT |
| | TGGTTCCACAATTGAGAACTGAAGAATTGTTGGCTGAAAGAG |
| | CTAGAGAATTGGGTGTTAGAATCGTTAGAGGTTCTGGTGTTA |
| | CTGGTTTCGCTCAAGACGCTGACGGTGTTACTGTTGAAACTG |
| | ACACTGGTTTGTTGAGAGCTAGATACTTGGTTGGTTGTGACG |
| | GTGGTTCTACTGTTAGAAAGGCTGCTGGTATCGGTTTCACTG |
| | GTCAAGACCCACACATGTACGCTTTGATCGGTGACATGAGAT |
| | TCTCTGGTGACTTGCCAAGAGGTGAAGGTTTGGGTCCAATGA |
| | GGCCAGTTGGTTTGGTTTACAGAGCTACTGTTGCTTGGTTCG |
| | ACAGACCATTCGCTGACAGAAGAGCGCCAGTTACTGAAGAA |
| | GAAATGAGAGCTGCTTTGGTTGAACACACTGGTTCTGACCAC |
| | GGTATGCACGACGTTACTTGGTTGTCTCGTTTGACTGACGTTT |
| | CTCGTTTGGCTGACTCTTACAGATTGGGTAGAGTTTTGTTGGC |
| | TGGTGACGCTGCTCACATCCACTTGCCAGCTGGTGGTCAAGG |
| | TTTGAACTTGGGTTTCCAAGACGCTGTTAACTTGGGTTGGAA |
| | GTTGGCTGCTGTTGTTAGAGGTCACGGTACTGAAGAATTGTT |
| | GGACTCTTACGGTAGAGAAAGAAGGCCAATCGCTGACGGTG |
| | TTGTTAGAAACACTAGAACTCAAGCTGTTTTGATCGACCCAG |
| | ACCCAAGATACGAAGCTCTCAGACAAACTTTGCCACACTTGA |
| | TGGCTTTGCCAGACACTAACAGACACATGGCTGGTATGTTGT |
| | CTGGTTTCGACGTTGCTTACGGTGGTGGTGACCACCCATTGG |
| | TTGGTAGAAGAATGCCAGACGCTGAATTGATCACTGCTGACG |
| | GTCCAAGAAGAATCTCTGACTGTTTCGCTGGTGCTAGAGGTT |
| | TGTTGTTGTTGCCAGAACAAGGTCCAACTGCTTCTCCATTGG |
| | CTGCTTGGGCTGACAGAGTTGACACTTTGACTGTTAAGTCTG |
| | GTGGTCCAGACCCAGACACTGCTCACTTGGTTAGACCAGACG |
| | GTTACGTTGCTTGGGCTGGTGAACCAGCTAGAACTGAAGAAT |
| | TGCACCACGCTGCTACTACTTGGTTCGGTGCTGCTGCTgtatcgat |
| | ggattacaaggatgacgacgataagatctgagctcttaattaacaattcttcgccagaggtttggtcaagt |
| | ctccaatcaaggttgtcggct (SEQ ID NO: 23). The capitalized sequence is |
| | SEQ ID NO: 45. |
| pTEF1-pgaE-tADH1 in | tcaagtttcagtttcatttttcttgttctattacaactttttttacttcttgctcattagaaagaaagcatagcaat |
| pSP-G1 JCAT codon | ctaatctaagttttaattacaagcggccgcATGGACGCTGCTGTTATCGTTGTTG |
| opt. (AL-1-234C-C1) | GTGCTGGTCCAGCTGGTATGATGTTGGCTGGTGAATTGAGATT |
| | GGCTGGTGTTGAAGTTGTTGTTTTGGAAAGATTGGTTGAAAGA |
| | ACTGGTGAATCTCGTGGTTTGGGTTTCACTGCTAGAACTATGG |
| | AAGTTTTCGACCAAAGAGGTATCTTGCCAAGATTCGGTGAAG |
| | TTGAAACTTCTACTCAAGGTCACTTCGGTGGTTTGCCAATCGA |
| | CTTCGGTGTTTTGGAAGGTGCTTGGCAAGCTGCTAAGACTGTT |
| | CCACAATCTGTTACTGAAACTCACTTGGAACAATGGGCTACTG |
| | GTTTGGGTGCTGACATCAGAAGAGGTCACGAAGTTTTGTCTTT |
| | GACTGACGACGGTGCTGGTGTTACTGTTGAAGTTAGAGGTCC |
| | AGAAGGTAAGCACACTTTGAGAGCTGCTTACTTGGTTGGTTGT |
| | GACGGTGGTAGATCGTCTGTTAGAAAGGCTGCTGGTTTCGACT |
| | TCCCAGGTACTGCTGCTACTATGGAAATGTACTTGGCTGACAT |
| | CAAGGGTGTTGAATTGCAACCAAGAATGATCGGTGAAACTTT |
| | GCCAGGTGGTATGGTTATGGTTGGTCCATTGCCAGGTGGTATC |
| | ACTAGAATCATCGTTTGTGAAAGAGGTACTCCACCACAAAGA |
| | AGAGAAACTCCACCATCTTGGCACGAAGTTGCTGACGCTTGG |
| | AAGAGATTGACTGGTGACGACATCGCTCACGCTGAACCAGTT |
| | TGGGTTTCTGCTTTCGGTAACGCTACTAGACAAGTTACTGAAT |
| | ACAGAAGAGGTAGAGTTATCTTGGCTGGTGACTCTGCTCACA |
| | TCCACTTGCCAGCTGGTGGTCAAGGTATGAACACTTCTATCCA |
| | AGACGCTGTTAACTTGGGTTGGAAGTTGGGTGCTGTTGTTAAC |
| | GGTACTGCTACTGAAGAATTGTTGGACTCTTACCACTCTGAAA |
| | GACACGCTGTTGGTAAGAGATTGTTGATGAACACTCAAGCTC |
| | AAGGTTTGTTGTTCTTGTCTGGTCCAGAAGTTCAACCATTGAG |
| | AGATGTTTTGACTGAATTGATCCAATACGGTGAAGTTGCTAGA |
| | CACTTGGCTGGTATGGTTTCTGGTTTGGAAATCACTTACGACG |
| | TTGGTACTGGTTCTCACCCATTGTTGGGTAAGAGAATGCCAGC |
| | TTTGGAATTGACTACTGCTACTAGAGAAACATCTTCTACTGAA |
| | TTATTACACACTGCTAGAGGTGTTTTGTTGGACTTGGCTGACA |
| | ACCCAAGATTGAGAGCTAGAGCTGCTGCTTGGTCTGACAGAG |
| | TTGACATCGTTACTGCTGTTCCAGGTGAAGTTTCTGCTACTTCT |
| | GGTTTGAGAGACACTACTGCTGTTTTGATAAGACCAGACGGT |
| | CACGTTGCTTGGGCTGCTCCAGGTTCTCACCACGACTTGCCAA |
| | TGGCTTTGGAAAGATGGTTCGGTGCTCCATTGACTGGTgtatcgat |
| | ggattacaaggatgacgacgataagatctgagctcttaattaacaattcttcgccagaggtttggtcaagt |
| | ctccaatcaaggttgtcggct (SEQ ID NO: 24). The capitalized sequence is |
| | SEQ ID NO: 46. |
| pTEF1-ssfO1-tADH1 in | tcaagtttcagtttcatttttcttgttctattacaactttttttacttcttgctcattagaaagaaagcatagcaa |
| pSP-G1 JCAT codon | tctaatctaagttttaattacaagcggccgcATGGAACCAGAAGTTGTTGTTGCTG |
| opt. (AL-1-239-B-C3) | GTGCTGGTCCAGTTGGTTTGATGTTGGCTTGTGAATTGAGAAG |
| | GCAAGGTGTTGGTGTTGTTTTGGTTGAAAGAACTACTGAACC |
| | AGCTAACGACAACAGAGCGCAAGCTCTCCACGGTAGAACTAT |
| | CCCAGTTTTGGACAGAAGAGGTTTGTTGCCAAGATTCAGAGC |
| | TGCTGAAAGAGAATTGAACGGTGGTGGTTCTGGTGGTACTGA |
| | ATCTCACAGAAAAAGACCATTGCCAAGAGGTCACTTCGCTGG |
| | TATCACTGGTTTATTACCATCTGCTCCAGACCCAGACCCAGAC |
| | GTTCCACCAGTTGTTTTCGTTCCACAATGGGTTACTACTAGAT |
| | TGTTGACTGAACACGCTGCTGAATTGGGTGTTGTTTTGCACAG |
| | AGGTTCTGAAATCACTGCTGTTGAACAAGACGCTGACGGTGT |
| | TACTGTTGCTGTTTCTGGTGGTACTGCTCCAGCTAGATTGAGA |
| | GCTAGATACTTGATAGGTTGTGACGGTGCTAGATCGGTTGTT |
| | AGAAGATCGGCTGGTATCGACTTCGCTGGTACTGGTGCTACT |
| | TTGTCTACTTTGGCTGCTGAAGTTCACTTGGACGACCCAGCTG |
| | ACTTGCCAGTTGGTTGGCACAGAACTCAACACGGTTGTACTG |
| | TTATCTCTCCACACCCAGAAGGTGGTCACTCTCGTGTTGTTGT |
| | TATCGACTTCTCTGGTCCAGACGCTGACAGAGAAGCTCCAGT |
| | TACTTTGACTGAATTGGAAGAAAGAATCGCTGGTGTTTTGGG |
| | TAGAAAGGTTGCTATGTCTGACTTGAAGAACGCTCAAAGATA |
| | CGGTGACGCTGCTAGATTGGCTGACAGATACAGAGTTGGTAG |
| | AGTTTTCTTGGCTGGTGACGCTGCTCACATCCACTACCCAGTT |
| | GGTGGTCAAGGTTTGAACATCGGTTTGCAAGACGCTGTTAAC |
| | TTGGCTTGGAAGTTGGCTGCTGACTTGAGAGGTTGGGCTCCA |
| | GACGGTTTGTTGGACACTTACCACACTGAAAGATACCCAGTT |
| | GCTGACTCTGTTTTGACTCACACTAGAGCGCAAGTTGCTTTGT |
| | TGGCTCCAGGTCCAAGAATCGACGCTTTGAGAACTTTGTTCA |
| | CTGACTTGATCGGTTTGGCTGACGTTTCTCGTTACTTGTCTGA |
| | AAAGATGTCTGCTGCTGACGTTAGATACGACATGGGTGAAGC |
| | TGAACCACACCCATTGACTGGTAGATTCGCTCCATCTTTGAG |
| | ATTGGCTACTGAACAAGGTGAAAGAAGATTGGCTGACTTGTT |
| | GGCTGGTGGTAGACCATTGTTGTTGGACTTGTCTGGTAGAGC |
| | TGCTACTGCTGCTGTTGCTGCTAGATGGGCTGACAGAGTTGA |
| | CGTTCACGCTGCTACTTGTCCAGCTGAACCAGCTTTGGGTTCT |
| | TTGTTGATCAGACCAGACTCTTACGTTGCTTGGGCTTGTGCTC |
| | CAGGTGACGCTCCAGACCAAGTTGAAAGAGGTTTGGAAGCT |
| | GCTTTGAGAAGATGGTTCGGTGCTCCAGACCACCAAgtatcgatgg |
| | attacaaggatgacgacgataagatctgagctcttaattaacaattcttcgccagaggtttggtcaagtct |
| | ccaatcaaggttgtcggct (SEQ ID NO: 25). The capitalized sequence is |
| | SEQ ID NO: 47. |
| pTEF1-UB14-dacO1- | tcaagtttcagtttcatttttcttgttctattacaactttttttacttcttgctcattagaaagaaagcatagcaa |
| tADH1 in pSP-G1 | tctaatctaagttttaattacaagcggccgccattATGCAGATCTTCGTCAAGACGTTA |
| | ACCGGTAAAACCATAACTCTAGAAGTTGAATCTTCCGATACC |
| | ATCGACAACGTTAAGTCGAAAATTCAAGACAAGGAAGGCATT |
| | CCACCTGATCAACAAAGATTGATCTTTGCCGGTAAGCAGCTC |
| | GAGGACGGTAGAACGCTGTCTGATTACAACATTCAGAAGGAG |
| | TCGACCTTACATCTTGTCTTAAGACTAAGAGGTGGTATGTTAG |
| | TAGCCGGTGGTGGACCAACAGGGTTATGGTTGGCTGGTGAAC |
| | TAAGGTTGGCCGGGGTTAGACCTCTTGTTTTAGAGCGTAGGAC |
| | AGAACCCTTTCCACATAGCAAAGCACTGGGTATCCATCCAAG |
| | AACTATTGAAATGTTGGAGTTGAGAGGCCTTGCTGGTAGATTT |
| | TTAGATGGCGCCCCGAAGCTGCCGAAAGGTCACTTTGCATCG |
| | CTTCCTGTGCCCTTAGACTATGGTGCAATGGATACTCGACATC |
| | CGTACGGTGTCTACCGTAAACAAGTGGAAACGGAAGCTTTAT |
| | TAGCCGGCCACGCAACCAGATTAGGAGTTCCAGTCCGCAGAG |
| | GTCATGAACTTGTAGGCCTAAGACAGCACGATGATGCTGTAG |
| | TCGGTACTGTTCAAGGTCCTCAAGGTAGATACGAAGTCAGAG |
| | CCGCTTATCTTGTTGGTTGTGATGGAGGAGGTTCTACCACAAG |
| | ACGCCTGGCTGGGATAGATTTCCCTGGACAAGATCCTGGTGTT |
| | GCCGTTTTGATGGCTGACGCAAGATTCCGGGACCCATTACCCT |
| | CTGATCCAGCTATGGGTCCATTCAGACGCTATGGAGTCCTGAG |
| | ACCTGATTTAAGGGCATGGTTTAGTGCTATTCCTTTACCAGAA |
| | GAACAGGGTTTATGCAGAGTTATGGTTTTTTGGTATGGACGAC |
| | AATTTGAAGATAGACGTGCTCCAGTAACTGAGGATGAAATGA |
| | GAGCAGCACTTGTTGATCTAGCGGGAACTGACTTTGGTATGTA |
| | CGATGTTCAATGGTTGACCAGATTCACAGACGCTTCCAGGCA |
| | AGCTGCAAGGTATAGATCTGGAAGGGTTTTCTTAGCTGGAGA |
| | TGCTGCTCATACTGTCTTCCCCACTGGTGGCCCAGGTTTGAAT |
| | GCAGGTTTACAAGATGCCATGAATTTAGGTTGGAAGTTAGCT |
| | GGAGCTGTTCATGGCTGGGGTGGTGCTTTACTAGATTCATATC |
| | ATGATGAGCGTTACCCTGTAGCTGAACAAGTTTTGCGTGACA |
| | CAAGAACACAATGTCTATTGTTAGATCCAGACCCTCGCTTTGT |
| | TCCATTAAGAGAGACGTTAGCAGAACTATTGAGACTAGAACC |
| | CGTAAACAGATATTTCACCGGACACAACACGGCTCTAGCAGT |
| | TAGATACGATTTGGGTGGTGCCCATCCTGCAACGGGAGGCAG |
| | AATGCCAGATGTCTTGGTCGCAACTGGTGCTGGTCAAAGAAG |
| | GTTTTCAGATTGTTGTGTTACCGGGAGAGGGGTTCTTTTAGTC |
| | ACTTCTGGTGCAGATAGGGGGGAACTGTTACGAGGATGGAAA |
| | GATAGAGTGGATGTGGTAACAGGTACCGTGGCAGGACCTCTT |
| | GATGCAGCTGATCATTTAGTTAGACCTGATGGTTATGTTGCAT |
| | GGGCCGGTGGAGCTGGACACGATGAACCTGGTTTAGAAACTG |
| | CTCTTAGATTATGGTTTGGTGAACCAGTTAGTgtatcgatggattacaag |
| | gatgacgacgataagatctgagctcttaattaacaattcttcgccagaggtttggtcaagtctccaatcaa |
| | ggttgtcggct (SEQ ID NO: 26). The capitalized sequence is SEQ ID |
| | NO: 48. |
| pTEF1-SOD1-dacO1- | tcaagtttcagtttcatttttcttgttctattacaactttttttacttcttgctcattagaaagaaagcatagcaa |
| tADH1 in pSP-G1 | tctaatctaagttttaattacaagcggccgccattATGGCGACGAAGGCCGTGTGCGT |
| | GCTGAAGGGCGACGGCCCAGTGCAGGGCATCATCAATTTCGA |
| | GCAGAAGGAAAGTAATGGACCAGTGAAGGTGTGGGGAAGCA |
| | TTAAAGGACTGACTGAAGGCCTGCATGGATTCCATGTTCATG |
| | AGTTTGGAGATAATACAGCAGGCTGTACCAGTGCAGGTCCTC |
| | ACTTTAATCCTCTATCCAGAAAACACGGTGGGCCAAAGGATG |
| | AAGAGAGGCATGTTGGAGACTTGGGCAATGTGACTGCTGACA |
| | AAGATGGTGTGGCCGATGTGTCTATTGAAGATTCTGTGATCTC |
| | ACTCTCAGGAGACCATTGCATCATTGGCCGCACACTGGTGGTC |
| | CATGAAAAAGCAGATGACTTGGGCAAAGGTGGAAATGAAGA |
| | AAGTACAAAGACAGGAAACGCTGGAAGTCGTTTGGCTTGTGG |
| | TGTAATTGGGATGTTAGTAGCCGGTGGTGGACCAACAGGGTT |
| | ATGGTTGGCTGGTGAACTAAGGTTGGCCGGGGTTAGACCTCTT |
| | GTTTTAGAGCGTAGGACAGAACCCTTTCCACATAGCAAAGCA |
| | CTGGGTATCCATCCAAGAACTATTGAAATGTTGGAGTTGAGA |
| | GGCCTTGCTGGTAGATTTTTAGATGGCGCCCCGAAGCTGCCGA |
| | AAGGTCACTTTGCATCGCTTCCTGTGCCCTTAGACTATGGTGC |
| | AATGGATACTCGACATCCGTACGGTGTCTACCGTAAACAAGT |
| | GGAAACGGAAGCTTTATTAGCCGGCCACGCAACCAGATTAGG |
| | AGTTCCAGTCCGCAGAGGTCATGAACTTGTAGGCCTAAGACA |
| | GCACGATGATGCTGTAGTCGGTACTGTTCAAGGTCCTCAAGGT |
| | AGATACGAAGTCAGAGCCGCTTATCTTGTTGGTTGTGATGGA |
| | GGAGGTTCTACCACAAGACGCCTGGCTGGGATAGATTTCCCT |
| | GGACAAGATCCTGGTGTTGCCGTTTTGATGGCTGACGCAAGA |
| | TTCCGGGACCCATTACCCTCTGATCCAGCTATGGGTCCATTCA |
| | GACGCTATGGAGTCCTGAGACCTGATTTAAGGGCATGGTTTA |
| | GTGCTATTCCTTTACCAGAAGAACAGGGTTTATGCAGAGTTAT |
| | GGTTTTTTGGTATGGACGACAATTTGAAGATAGACGTGCTCCA |
| | GTAACTGAGGATGAAATGAGAGCAGCACTTGTTGATCTAGCG |
| | GGAACTGACTTTGGTATGTACGATGTTCAATGGTTGACCAGAT |
| | TCACAGACGCTTCCAGGCAAGCTGCAAGGTATAGATCTGGAA |
| | GGGTTTTCTTAGCTGGAGATGCTGCTCATACTGTCTTCCCCAC |
| | TGGTGGCCCAGGTTTGAATGCAGGTTTACAAGATGCCATGAA |
| | TTTAGGTTGGAAGTTAGCTGGAGCTGTTCATGGCTGGGGTGGT |
| | GCTTTACTAGATTCATATCATGATGAGCGTTACCCTGTAGCTG |
| | AACAAGTTTTGCGTGACACAAGAACACAATGTCTATTGTTAG |
| | ATCCAGACCCTCGCTTTGTTCCATTAAGAGAGACGTTAGCAGA |
| | ACTATTGAGACTAGAACCCGTAAACAGATATTTCACCGGACA |
| | CAACACGGCTCTAGCAGTTAGATACGATTTGGGTGGTGCCCA |
| | TCCTGCAACGGGAGGCAGAATGCCAGATGTCTTGGTCGCAAC |
| | TGGTGCTGGTCAAAGAAGGTTTTCAGATTGTTGTGTTACCGGG |
| | AGAGGGGTTCTTTTAGTCACTTCTGGTGCAGATAGGGGGGAA |
| | CTGTTACGAGGATGGAAAGATAGAGTGGATGTGGTAACAGGT |
| | ACCGTGGCAGGACCTCTTGATGCAGCTGATCATTTAGTTAGAC |
| | CTGATGGTTATGTTGCATGGGCCGGTGGAGCTGGACACGATG |
| | AACCTGGTTTAGAAACTGCTCTTAGATTATGGTTTGGTGAACC |
| | AGTTAGTgtatcgatggattacaaggatgacgacgataagatctgagctcttaattaacaattcttc |
| | gccagaggtttggtcaagtctccaatcaaggttgtcggct (SEQ ID NO: 27). The |
| | capitalized sequence is SEQ ID NO: 49. |
| pTEF1-GAL1-dacO1- | tcaagtttcagtttcatttttcttgttctattacaactttttttacttcttgctcattagaaagaaagcatagcaa |
| tADH1 in pSP-G1 | tctaatctaagttttaattacaagcggccgcATGACTAAATCTCATTCAGAAGAA |
| | GTGATTGTACCTGAGTTCAATTCTAGCGCAAAGGAATTACCA |
| | AGACCATTGGCCGAAAAGTGCCCGAGCATAATTAAGAAATT |
| | TATAAGCGCTTATGATGCTAAACCGGATTTTGTTGCTAGATC |
| | GCCTGGTAGAGTCAATCTAATTGGTGAACATATTGATTATTG |
| | TGACTTCTCGGTTTTACCTTTAGCTATTGATTTTGATATGCTTT |
| | GCGCCGTCAAAGTTTTGAACGAGAAAAATCCATCCATTACCT |
| | TAATAAATGCTGATCCCAAATTTGCTCAAAGGAAGTTCGATT |
| | TGCCGTTGGACGGTTCTTATGTCACAATTGATCCTTCTGTGTC |
| | GGACTGGTCTAATTACTTTAAATGTGGTCTCCATGTTGCTCAC |
| | TCTTTTCTAAAGAAACTTGCACCGGAAAGGTTTGCCAGTGCT |
| | CCTCTGGCCGGGCTGCAAGTCTTCTGTGAGGGTGATGTACCA |
| | ACTGGCAGTGGATTGTCTTCTTCGGCCGCATTCATTTGTGCCG |
| | TTGCTTTAGCTGTTGTTAAAGCGAATATGGGCCCTGGTTATC |
| | ATATGTCCAAGCAAAATTTAATGCGTATTACGGTCGTTGCAG |
| | AACATTATGTTGGTGTTAACAATGGCGGTATGGATCAGGCTG |
| | CCTCTGTTTGCGGTGAGGAAGATCATGCTCTATACGTTGAGT |
| | TCAAACCGCAGTTGAAGGCTACTCCGTTTAAATTTCCGCAAT |
| | TAAAAAACCATGAAATTAGCTTTGTTATTGCGAACACCCTTG |
| | TTGTATCTAACAAGTTTGAAACCGCCCCAACCAACTATAATT |
| | TAAGAGTGGTAGAAGTCACTACAGaattccggATGTTAGTAGCCG |
| | GTGGTGGACCAACAGGGTTATGGTTGGCTGGTGAACTAAGGT |
| | TGGCCGGGGTTAGACCTCTTGTTTTAGAGCGTAGGACAGAAC |
| | CCTTTCCACATAGCAAAGCACTGGGTATCCATCCAAGAACTA |
| | TTGAAATGTTGGAGTTGAGAGGCCTTGCTGGTAGATTTTTAG |
| | ATGGCGCCCCGAAGCTGCCGAAAGGTCACTTTGCATCGCTTC |
| | CTGTGCCCTTAGACTATGGTGCAATGGATACTCGACATCCGT |
| | ACGGTGTCTACCGTAAACAAGTGGAAACGGAAGCTTTATTAG |
| | CCGGCCACGCAACCAGATTAGGAGTTCCAGTCCGCAGAGGT |
| | CATGAACTTGTAGGCCTAAGACAGCACGATGATGCTGTAGTC |
| | GGTACTGTTCAAGGTCCTCAAGGTAGATACGAAGTCAGAGCC |
| | GCTTATCTTGTTGGTTGTGATGGAGGAGGTTCTACCACAAGA |
| | CGCCTGGCTGGGATAGATTTCCCTGGACAAGATCCTGGTGTT |
| | GCCGTTTTGATGGCTGACGCAAGATTCCGGGACCCATTACCC |
| | TCTGATCCAGCTATGGGTCCATTCAGACGCTATGGAGTCCTG |
| | AGACCTGATTTAAGGGCATGGTTTAGTGCTATTCCTTTACCA |
| | GAAGAACAGGGTTTATGCAGAGTTATGGTTTTTTGGTATGGAC |
| | GACAATTTGAAGATAGACGTGCTCCAGTAACTGAGGATGAAA |
| | TGAGAGCAGCACTTGTTGATCTAGCGGGAACTGACTTTGGTAT |
| | GTACGATGTTCAATGGTTGACCAGATTCACAGACGCTTCCAG |
| | GCAAGCTGCAAGGTATAGATCTGGAAGGGTTTTCTTAGCTGG |
| | AGATGCTGCTCATACTGTCTTCCCCACTGGTGGCCCAGGTTTG |
| | AATGCAGGTTTACAAGATGCCATGAATTTAGGTTGGAAGTTA |
| | GCTGGAGCTGTTCATGGCTGGGGTGGTGCTTTACTAGATTCAT |
| | ATCATGATGAGCGTTACCCTGTAGCTGAACAAGTTTTGCGTGA |
| | CACAAGAACACAATGTCTATTGTTAGATCCAGACCCTCGCTTT |
| | GTTCCATTAAGAGAGACGTTAGCAGAACTATTGAGACTAGAA |
| | CCCGTAAACAGATATTTCACCGGACACAACACGGCTCTAGCA |
| | GTTAGATACGATTTGGGTGGTGCCCATCCTGCAACGGGAGGC |
| | AGAATGCCAGATGTCTTGGTCGCAACTGGTGCTGGTCAAAGA |
| | AGGTTTTCAGATTGTTGTGTTACCGGGAGAGGGGTTCTTTTAG |
| | TCACTTCTGGTGCAGATAGGGGGGAACTGTTACGAGGATGGA |
| | AAGATAGAGTGGATGTGGTAACAGGTACCGTGGCAGGACCTC |
| | TTGATGCAGCTGATCATTTAGTTAGACCTGATGGTTATGTTGC |
| | ATGGGCCGGTGGAGCTGGACACGATGAACCTGGTTTAGAAAC |
| | TGCTCTTAGATTATGGTTTGGTGAACCAGTTAGTgtatcgatggattaca |
| | aggatgacgacgataagatctgagctcttaattaacaattcttcgccagaggtttggtcaagtctccaatca |
| | aggttgtcggct (SEQ ID NO: 28). The capitalized sequence is SEQ ID |
| | NO: 50. |
| pGAL1-dacO1-tCYC1 in | ccctcactaaagggaacaaaagctggagctctagtacggattagaagccgccgagcgggtgacag |
| pRS416 andpGALl- | ccctccgaaggaagactctcctccgtgcgtcctcgtcttcaccggtcgcgttcctgaaacgcagatgt |
| dacO1-tCYC1 in | gcctcgcgccgcactgctccgaacaataaagattctacaatactagcttttatggttatgaagaggaaa |
| pRS426 | aattggcagtaacctggccccacaaaccttcaaatgaacgaatcaaattaacaaccataggatgataat |
| | gcgattagttttttagccttatttctggggtaattaatcagcgaagcgatgatttttgatctattaacagatat |
| | ataaatgcaaaaactgcataaccactttaactaatactttcaacattttcggtttgtattacttcttattcaaat |
| | gtaataaaagtatcaacaaaaaattgttaatatacctctatactttaacgtcaaggagaaaaaaccccgg |
| | attctagaactagtATGTTAGTAGCCGGTGGTGGACCAACAGGGTTAT |
| | GGTTGGCTGGTGAACTAAGGTTGGCCGGGGTTAGACCTCTTG |
| | TTTTAGAGCGTAGGACAGAACCCTTTCCACATAGCAAAGCAC |
| | TGGGTATCCATCCAAGAACTATTGAAATGTTGGAGTTGAGAG |
| | GCCTTGCTGGTAGATTTTTAGATGGCGCCCCGAAGCTGCCGA |
| | AAGGTCACTTTGCATCGCTTCCTGTGCCCTTAGACTATGGTGC |
| | AATGGATACTCGACATCCGTACGGTGTCTACCGTAAACAAGT |
| | GGAAACGGAAGCTTTATTAGCCGGCCACGCAACCAGATTAG |
| | GAGTTCCAGTCCGCAGAGGTCATGAACTTGTAGGCCTAAGAC |
| | AGCACGATGATGCTGTAGTCGGTACTGTTCAAGGTCCTCAAG |
| | GTAGATACGAAGTCAGAGCCGCTTATCTTGTTGGTTGTGATG |
| | GAGGAGGTTCTACCACAAGACGCCTGGCTGGGATAGATTTCC |
| | CTGGACAAGATCCTGGTGTTGCCGTTTTGATGGCTGACGCAA |
| | GATTCCGGGACCCATTACCCTCTGATCCAGCTATGGGTCCAT |
| | TCAGACGCTATGGAGTCCTGAGACCTGATTTAAGGGCATGGT |
| | TTAGTGCTATTCCTTTACCAGAAGAACAGGGTTTATGCAGAG |
| | TTATGGTTTTTTGGTATGGACGACAATTTGAAGATAGACGTG |
| | CTCCAGTAACTGAGGATGAAATGAGAGCAGCACTTGTTGATC |
| | TAGCGGGAACTGACTTTGGTATGTACGATGTTCAATGGTTGA |
| | CCAGATTCACAGACGCTTCCAGGCAAGCTGCAAGGTATAGAT |
| | CTGGAAGGGTTTTCTTAGCTGGAGATGCTGCTCATACTGTCTT |
| | CCCCACTGGTGGCCCAGGTTTGAATGCAGGTTTACAAGATGC |
| | CATGAATTTAGGTTGGAAGTTAGCTGGAGCTGTTCATGGCTG |
| | GGGTGGTGCTTTACTAGATTCATATCATGATGAGCGTTACCCT |
| | GTAGCTGAACAAGTTTTGCGTGACACAAGAACACAATGTCTA |
| | TTGTTAGATCCAGACCCTCGCTTTGTTCCATTAAGAGAGACGT |
| | TAGCAGAACTATTGAGACTAGAACCCGTAAACAGATATTTCA |
| | CCGGACACAACACGGCTCTAGCAGTTAGATACGATTTGGGTG |
| | GTGCCCATCCTGCAACGGGAGGCAGAATGCCAGATGTCTTGG |
| | TCGCAACTGGTGCTGGTCAAAGAAGGTTTTCAGATTGTTGTGT |
| | TACCGGGAGAGGGGTTCTTTTAGTCACTTCTGGTGCAGATAG |
| | GGGGGAACTGTTACGAGGATGGAAAGATAGAGTGGATGTGG |
| | TAACAGGTACCGTGGCAGGACCTCTTGATGCAGCTGATCATT |
| | TAGTTAGACCTGATGGTTATGTTGCATGGGCCGGTGGAGCTG |
| | GACACGATGAACCTGGTTTAGAAACTGCTCTTAGATTATGGT |
| | TTGGTGAACCAGTTAGTgtatcgatggattacaaggatgacgacgataagatctgaaa |
| | gcttatcgataccgtcgacctcgagtcatgtaattagttatgtcacgcttacattcacgccctccccccac |
| | atccgctctaaccgaaaaggaaggagttagacaacctgaagtctaggtccctatttatttttttatagttatg |
| | ttagtattaagaacgttatttatatttcaaatttttcttttttttctgtacagacgcgtgtacgcatgtaacatt |
| | atactgaaaaccttgcttgagaaggttttgggacgctcgaaggctttaatttgcggccggtacccaat |
| | (SEQ ID NO: 29). The capitalized sequence is SEQ ID NO: 51. |
| pTEF1-dacO1-tADH1 in | tcaagtttcagtttcatttttcttgttctattacaactttttttacttcttgctcattagaaagaaagcatagca |
| pSP-G1 (no antibody | atctaatctaagttttaattacaagcggccgCATGTTAGTAGCCGGTGGTGGACCAA |
| tag) | CAGGGTTATGGTTGGCTGGTGAACTAAGGTTGGCCGGGGTTA |
| | GACCTCTTGTTTTAGAGCGTAGGACAGAACCCTTTCCACATAG |
| | CAAAGCACTGGGTATCCATCCAAGAACTATTGAAATGTTGGA |
| | GTTGAGAGGCCTTGCTGGTAGATTTTTAGATGGCGCCCCGAA |
| | GCTGCCGAAAGGTCACTTTGCATCGCTTCCTGTGCCCTTAGAC |
| | TATGGTGCAATGGATACTCGACATCCGTACGGTGTCTACCGTA |
| | AACAAGTGGAAACGGAAGCTTTATTAGCCGGCCACGCAACCA |
| | GATTAGGAGTTCCAGTCCGCAGAGGTCATGAACTTGTAGGCC |
| | TAAGACAGCACGATGATGCTGTAGTCGGTACTGTTCAAGGTC |
| | CTCAAGGTAGATACGAAGTCAGAGCCGCTTATCTTGTTGGTTG |
| | TGATGGAGGAGGTTCTACCACAAGACGCCTGGCTGGGATAGA |
| | TTTCCCTGGACAAGATCCTGGTGTTGCCGTTTTGATGGCTGAC |
| | GCAAGATTCCGGGACCCATTACCCTCTGATCCAGCTATGGGTC |
| | CATTCAGACGCTATGGAGTCCTGAGACCTGATTTAAGGGCAT |
| | GGTTTAGTGCTATTCCTTTACCAGAAGAACAGGGTTTATGCAG |
| | AGTTATGGTTTTTTGGTATGGACGACAATTTGAAGATAGACGT |
| | GCTCCAGTAACTGAGGATGAAATGAGAGCAGCACTTGTTGAT |
| | CTAGCGGGAACTGACTTTGGTATGTACGATGTTCAATGGTTGA |
| | CCAGATTCACAGACGCTTCCAGGCAAGCTGCAAGGTATAGAT |
| | CTGGAAGGGTTTTCTTAGCTGGAGATGCTGCTCATACTGTCTT |
| | CCCCACTGGTGGCCCAGGTTTGAATGCAGGTTTACAAGATGC |
| | CATGAATTTAGGTTGGAAGTTAGCTGGAGCTGTTCATGGCTGG |
| | GGTGGTGCTTTACTAGATTCATATCATGATGAGCGTTACCCTG |
| | TAGCTGAACAAGTTTTGCGTGACACAAGAACACAATGTCTAT |
| | TGTTAGATCCAGACCCTCGCTTTGTTCCATTAAGAGAGACGTT |
| | AGCAGAACTATTGAGACTAGAACCCGTAAACAGATATTTCAC |
| | CGGACACAACACGGCTCTAGCAGTTAGATACGATTTGGGTGG |
| | TGCCCATCCTGCAACGGGAGGCAGAATGCCAGATGTCTTGGT |
| | CGCAACTGGTGCTGGTCAAAGAAGGTTTTCAGATTGTTGTGTT |
| | ACCGGGAGAGGGGTTCTTTTAGTCACTTCTGGTGCAGATAGG |
| | GGGGAACTGTTACGAGGATGGAAAGATAGAGTGGATGTGGTA |
| | ACAGGTACCGTGGCAGGACCTCTTGATGCAGCTGATCATTTA |
| | GTTAGACCTGATGGTTATGTTGCATGGGCCGGTGGAGCTGGA |
| | CACGATGAACCTGGTTTAGAAACTGCTCTTAGATTATGGTTTG |
| | GTGAACCAGTTAGTTGAgagctcttaattaacaattcttcgccagaggtttggtcaagtct |
| | ccaatcaaggttgtcggcttgtctaccttgccagaaatttacgaaaagatggaaaagggtcaaatcgttg |
| | gtagatacgttgttgacacttctaaataagcgaatttcttatgatttatgatttttattattaaataagttataaa |
| | aaaaataagtgtatacaaattttaaagtgactcttaggttttaaaacgaaaattcttattcttgagtaactcttt |
| | cctgtaggtcaggttgctttctcaggtatagcatgaggtcgctccaattcagctggcgtaatagcgaaga |
| | (SEQ ID NO: 30). The capitalized sequence is SEQ ID NO: 52. |
| pADH2-dacO1-tCYC1 | ccctcactaaagggaacaaaagctggagctcGCAAAACGTAGGGGCAAACAAA |
| in pRS416 and pADH2- | CGGAAAAATCGTTTCTCAAATTTTCTGATGCCAAGAACTCTAA |
| dacO1-tCYC1 in | CCAGTCTTATCTAAAAATTGCCTTATGATCCGTCTCTCCGGTT |
| pRS426 | ACAGCCTGTGTAACTGATTAATCCTGCCTTTCTAATCACCATT |
| | CTAATGTTTTAATTAAGGGATTTTGTCTTCATTAACGGCTTTCG |
| | CTCATAAAAATGTTATGACGTTTTGCCCGCAGGCGGGAAACC |
| | ATCCACTTCACGAGACTGATCTCCTCTGCCGGAACACCGGGC |
| | ATCTCCAACTTATAAGTTGGAGAAATAAGAGAATTTCAGATT |
| | GAGAGAATGAAAAAAAAAAAAAAAAAAAAGGCAGAGGAGA |
| | GCATAGAAATGGGGTTCACTTTTTGGTAAAGCTATAGCATGCC |
| | TATCACATATAAATAGAGTGCCAGTAGCGACTTTTTTCACACT |
| | CGAAATACTCTTACTACTGCTCTCTTGTTGTTTTTATCACTTCT |
| | TGTTTCTTCTTGGTAAATAGAATATCAAGCTACAAAAAGCATA |
| | CAATCAACTATCAACTATTAACTATATCGTAATACCATATGGC |
| | TAtctagaactagtATGTTAGTAGCCGGTGGTGGACCAACAGGGTTA |
| | TGGTTGGCTGGTGAACTAAGGTTGGCCGGGGTTAGACCTCTTG |
| | TTTTAGAGCGTAGGACAGAACCCTTTCCACATAGCAAAGCAC |
| | TGGGTATCCATCCAAGAACTATTGAAATGTTGGAGTTGAGAG |
| | GCCTTGCTGGTAGATTTTTAGATGGCGCCCCGAAGCTGCCGAA |
| | AGGTCACTTTGCATCGCTTCCTGTGCCCTTAGACTATGGTGCA |
| | ATGGATACTCGACATCCGTACGGTGTCTACCGTAAACAAGTG |
| | GAAACGGAAGCTTTATTAGCCGGCCACGCAACCAGATTAGGA |
| | GTTCCAGTCCGCAGAGGTCATGAACTTGTAGGCCTAAGACAG |
| | CACGATGATGCTGTAGTCGGTACTGTTCAAGGTCCTCAAGGTA |
| | GATACGAAGTCAGAGCCGCTTATCTTGTTGGTTGTGATGGAG |
| | GAGGTTCTACCACAAGACGCCTGGCTGGGATAGATTTCCCTG |
| | GACAAGATCCTGGTGTTGCCGTTTTGATGGCTGACGCAAGATT |
| | CCGGGACCCATTACCCTCTGATCCAGCTATGGGTCCATTCAGA |
| | CGCTATGGAGTCCTGAGACCTGATTTAAGGGCATGGTTTAGTG |
| | CTATTCCTTTACCAGAAGAACAGGGTTTATGCAGAGTTATGGT |
| | TTTTTGGTATGGACGACAATTTGAAGATAGACGTGCTCCAGTA |
| | ACTGAGGATGAAATGAGAGCAGCACTTGTTGATCTAGCGGGA |
| | ACTGACTTTGGTATGTACGATGTTCAATGGTTGACCAGATTCA |
| | CAGACGCTTCCAGGCAAGCTGCAAGGTATAGATCTGGAAGGG |
| | TTTTCTTAGCTGGAGATGCTGCTCATACTGTCTTCCCCACTGGT |
| | GGCCCAGGTTTGAATGCAGGTTTACAAGATGCCATGAATTTA |
| | GGTTGGAAGTTAGCTGGAGCTGTTCATGGCTGGGGTGGTGCTT |
| | TACTAGATTCATATCATGATGAGCGTTACCCTGTAGCTGAACA |
| | AGTTTTGCGTGACACAAGAACACAATGTCTATTGTTAGATCCA |
| | GACCCTCGCTTTGTTCCATTAAGAGAGACGTTAGCAGAACTAT |
| | TGAGACTAGAACCCGTAAACAGATATTTCACCGGACACAACA |
| | CGGCTCTAGCAGTTAGATACGATTTGGGTGGTGCCCATCCTGC |
| | AACGGGAGGCAGAATGCCAGATGTCTTGGTCGCAACTGGTGC |
| | TGGTCAAAGAAGGTTTTCAGATTGTTGTGTTACCGGGAGAGG |
| | GGTTCTTTTAGTCACTTCTGGTGCAGATAGGGGGGAACTGTTA |
| | CGAGGATGGAAAGATAGAGTGGATGTGGTAACAGGTACCGT |
| | GGCAGGACCTCTTGATGCAGCTGATCATTTAGTTAGACCTGA |
| | TGGTTATGTTGCATGGGCCGGTGGAGCTGGACACGATGAACC |
| | TGGTTTAGAAACTGCTCTTAGATTATGGTTTGGTGAACCAGTT |
| | AGTgtatcgatggattacaaggatgacgacgataagatctgaaagcttatcgataccgtcgacctcg |
| | agtcatgtaattagttatgtcacgcttacattcacgccctccccccacatccgctctaaccgaaaaggaa |
| | ggagttagacaacctgaagtctaggtccctatttatttttttatagttatgttagtattaagaacgttatttatat |
| | ttcaaatttttcttttttttctgtacagacgcgtgtacgcatgtaacattatactgaaaaccttgcttgagaagg |
| | ttttgggacgctcgaaggctttaatttgcggccggtacccaat (SEQ ID NO: 31). The |
| | capitalized sequence is SEQ ID NO: 53. |
| pTEF1-UBI4-IFNG- | tcaagtttcagtttcatttttcttgttctattacaactttttttacttcttgctcattagaaagaaagcatagcaa |
| dacO1-tADH1 inpSP- | tctaatctaagttttaattacaagcggccgccattATGCAGATCTTCGTCAAGACGT |
| G1 | TAACCGGTAAAACCATAACTCTAGAAGTTGAATCTTCCGATA |
| | CCATCGACAACGTTAAGTCGAAAATTCAAGACAAGGAAGGC |
| | ATTCCACCTGATCAACAAAGATTGATCTTTGCCGGTAAGCAG |
| | CTCGAGGACGGTAGAACGCTGTCTGATTACAACATTCAGAAG |
| | GAGTCGACCTTACATCTTGTCTTAAGACTAAGAGGTGGTATG |
| | CAAGACCCATATGTAAAAGAAGCAGAAAACCTTAAGAAATA |
| | TTTTAATGCAGGTCATTCAGATGTAGCGGATAATGGAACTCT |
| | TTTCTTAGGCATTTTGAAGAATTGGAAAGAGGAGAGTGACAG |
| | AAAAATAATGCAGAGCCAAATTGTCTCCTTTTACTTCAAACT |
| | TTTTAAAAACTTTAAAGATGACCAGAGCATCCAAAAGAGTGT |
| | GGAGACCATCAAGGAAGACATGAATGTCAAGTTTTTCAATA |
| | GCAACAAAAAGAAACGAGATGACTTCGAAAAGCTGACTAAT |
| | TATTCGGTAACTGACTTGAATGTCCAACGCAAAGCAATACAT |
| | GAACTCATCCAAGTGATGGCTGAACTGTCGCCAGCAGCTAAA |
| | ACAGGGAAGCGAAAAAGGAGTCAGATGCTGTTTCGAGGTCG |
| | AAGAGCATCCCAGATGTTAGTAGCCGGTGGTGGACCAACAG |
| | GGTTATGGTTGGCTGGTGAACTAAGGTTGGCCGGGGTTAGAC |
| | CTCTTGTTTTAGAGCGTAGGACAGAACCCTTTCCACATAGCA |
| | AAGCACTGGGTATCCATCCAAGAACTATTGAAATGTTGGAGT |
| | TGAGAGGCCTTGCTGGTAGATTTTTAGATGGCGCCCCGAAGC |
| | TGCCGAAAGGTCACTTTGCATCGCTTCCTGTGCCCTTAGACT |
| | ATGGTGCAATGGATACTCGACATCCGTACGGTGTCTACCGTA |
| | AACAAGTGGAAACGGAAGCTTTATTAGCCGGCCACGCAACC |
| | AGATTAGGAGTTCCAGTCCGCAGAGGTCATGAACTTGTAGGC |
| | CTAAGACAGCACGATGATGCTGTAGTCGGTACTGTTCAAGGT |
| | CCTCAAGGTAGATACGAAGTCAGAGCCGCTTATCTTGTTGGT |
| | TGTGATGGAGGAGGTTCTACCACAAGACGCCTGGCTGGGAT |
| | AGATTTCCCTGGACAAGATCCTGGTGTTGCCGTTTTGATGGC |
| | TGACGCAAGATTCCGGGACCCATTACCCTCTGATCCAGCTAT |
| | GGGTCCATTCAGACGCTATGGAGTCCTGAGACCTGATTTAAG |
| | GGCATGGTTTAGTGCTATTCCTTTACCAGAAGAACAGGGTTT |
| | ATGCAGAGTTATGGTTTTTTGGTATGGACGACAATTTGAAGA |
| | TAGACGTGCTCCAGTAACTGAGGATGAAATGAGAGCAGCAC |
| | TTGTTGATCTAGCGGGAACTGACTTTGGTATGTACGATGTTC |
| | AATGGTTGACCAGATTCACAGACGCTTCCAGGCAAGCTGCAA |
| | GGTATAGATCTGGAAGGGTTTTCTTAGCTGGAGATGCTGCTC |
| | ATACTGTCTTCCCCACTGGTGGCCCAGGTTTGAATGCAGGTT |
| | TACAAGATGCCATGAATTTAGGTTGGAAGTTAGCTGGAGCTG |
| | TTCATGGCTGGGGTGGTGCTTTACTAGATTCATATCATGATG |
| | AGCGTTACCCTGTAGCTGAACAAGTTTTGCGTGACACAAGAA |
| | CACAATGTCTATTGTTAGATCCAGACCCTCGCTTTGTTCCATT |
| | AAGAGAGACGTTAGCAGAACTATTGAGACTAGAACCCGTAAA |
| | CAGATATTTCACCGGACACAACACGGCTCTAGCAGTTAGATA |
| | CGATTTGGGTGGTGCCCATCCTGCAACGGGAGGCAGAATGCC |
| | AGATGTCTTGGTCGCAACTGGTGCTGGTCAAAGAAGGTTTTCA |
| | GATTGTTGTGTTACCGGGAGAGGGGTTCTTTTAGTCACTTCTG |
| | GTGCAGATAGGGGGGAACTGTTACGAGGATGGAAAGATAGA |
| | GTGGATGTGGTAACAGGTACCGTGGCAGGACCTCTTGATGCA |
| | GCTGATCATTTAGTTAGACCTGATGGTTATGTTGCATGGGCCG |
| | GTGGAGCTGGACACGATGAACCTGGTTTAGAAACTGCTCTTA |
| | GATTATGGTTTGGTGAACCAGTTAGTgtatcgatggattacaaggatgacgac |
| | gataagatctgagctcttaattaacaattcttcgccagaggtttggtcaagtctccaatcaaggttgtcggct |
| | (SEQ ID NO: 32). The capitalized sequence is SEQ ID NO: 54. |
| pTEFl1-IFNG-dacO1- | tcaagtttcagtttcatttttcttgttctattacaactttttttacttcttgctcattagaaagaaagcatagcaa |
| tADH1 in pSP-G1 | tctaatctaagttttaattacaagcggccgccattATGCAAGACCCATATGTAAAAGA |
| | AGCAGAAAACCTTAAGAAATATTTTAATGCAGGTCATTCAGA |
| | TGTAGCGGATAATGGAACTCTTTTCTTAGGCATTTTGAAGAAT |
| | TGGAAAGAGGAGAGTGACAGAAAAATAATGCAGAGCCAAAT |
| | TGTCTCCTTTTACTTCAAACTTTTTAAAAACTTTAAAGATGACC |
| | AGAGCATCCAAAAGAGTGTGGAGACCATCAAGGAAGACATG |
| | AATGTCAAGTTTTTCAATAGCAACAAAAAGAAACGAGATGAC |
| | TTCGAAAAGCTGACTAATTATTCGGTAACTGACTTGAATGTCC |
| | AACGCAAAGCAATACATGAACTCATCCAAGTGATGGCTGAAC |
| | TGTCGCCAGCAGCTAAAACAGGGAAGCGAAAAAGGAGTCAG |
| | ATGCTGTTTCGAGGTCGAAGAGCATCCCAGATGTTAGTAGCC |
| | GGTGGTGGACCAACAGGGTTATGGTTGGCTGGTGAACTAAGG |
| | TTGGCCGGGGTTAGACCTCTTGTTTTAGAGCGTAGGACAGAA |
| | CCCTTTCCACATAGCAAAGCACTGGGTATCCATCCAAGAACT |
| | ATTGAAATGTTGGAGTTGAGAGGCCTTGCTGGTAGATTTTTAG |
| | ATGGCGCCCCGAAGCTGCCGAAAGGTCACTTTGCATCGCTTCC |
| | TGTGCCCTTAGACTATGGTGCAATGGATACTCGACATCCGTAC |
| | GGTGTCTACCGTAAACAAGTGGAAACGGAAGCTTTATTAGCC |
| | GGCCACGCAACCAGATTAGGAGTTCCAGTCCGCAGAGGTCAT |
| | GAACTTGTAGGCCTAAGACAGCACGATGATGCTGTAGTCGGT |
| | ACTGTTCAAGGTCCTCAAGGTAGATACGAAGTCAGAGCCGCT |
| | TATCTTGTTGGTTGTGATGGAGGAGGTTCTACCACAAGACGCC |
| | TGGCTGGGATAGATTTCCCTGGACAAGATCCTGGTGTTGCCGT |
| | TTTGATGGCTGACGCAAGATTCCGGGACCCATTACCCTCTGAT |
| | CCAGCTATGGGTCCATTCAGACGCTATGGAGTCCTGAGACCT |
| | GATTTAAGGGCATGGTTTAGTGCTATTCCTTTACCAGAAGAAC |
| | AGGGTTTATGCAGAGTTATGGTTTTTTGGTATGGACGACAATT |
| | TGAAGATAGACGTGCTCCAGTAACTGAGGATGAAATGAGAGC |
| | AGCACTTGTTGATCTAGCGGGAACTGACTTTGGTATGTACGAT |
| | GTTCAATGGTTGACCAGATTCACAGACGCTTCCAGGCAAGCT |
| | GCAAGGTATAGATCTGGAAGGGTTTTCTTAGCTGGAGATGCT |
| | GCTCATACTGTCTTCCCCACTGGTGGCCCAGGTTTGAATGCAG |
| | GTTTACAAGATGCCATGAATTTAGGTTGGAAGTTAGCTGGAG |
| | CTGTTCATGGCTGGGGTGGTGCTTTACTAGATTCATATCATGA |
| | TGAGCGTTACCCTGTAGCTGAACAAGTTTTGCGTGACACAAG |
| | AACACAATGTCTATTGTTAGATCCAGACCCTCGCTTTGTTCCA |
| | TTAAGAGAGACGTTAGCAGAACTATTGAGACTAGAACCCGTA |
| | AACAGATATTTCACCGGACACAACACGGCTCTAGCAGTTAGA |
| | TACGATTTGGGTGGTGCCCATCCTGCAACGGGAGGCAGAATG |
| | CCAGATGTCTTGGTCGCAACTGGTGCTGGTCAAAGAAGGTTT |
| | TCAGATTGTTGTGTTACCGGGAGAGGGGTTCTTTTAGTCACTT |
| | CTGGTGCAGATAGGGGGGAACTGTTACGAGGATGGAAAGAT |
| | AGAGTGGATGTGGTAACAGGTACCGTGGCAGGACCTCTTGAT |
| | GCAGCTGATCATTTAGTTAGACCTGATGGTTATGTTGCATGGG |
| | CCGGTGGAGCTGGACACGATGAACCTGGTTTAGAAACTGCTC |
| | TTAGATTATGGTTTGGTGAACCAGTTAGTgtatcgatggattacaaggatga |
| | cgacgataagatctgagctcttaattaacaattcttcgccagaggtttggtcaagtctccaatcaaggttgt |
| | cggct (SEQ ID NO: 33). The capitalized sequence is SEQ ID NO: 55. |
| pPGK1-dacO4-dacO1- | tggaatggcgggaaagggtttagtaccacatgctatgatgcccactgtgatctccagagcaaagttcg |
| tCYC1 inpSP-G1 | ttcgatcgtactgttactctctctctttcaaacagaattgtccgaatcgtgtgacaacaacagcctgttctc |
| | acacactcttttcttctaaccaagggggtggtttagtttagtagaacctcgtgaaacttacatttacatatat |
| | ataaacttgcataaattggtcaatgcaagaaatacatatttggtcttttctaattcgtagtttttcaagttctta |
| | gatgctttctttttctcttttttacagatcatcaaggaagtaattatctactttttacaacaaatataaaacaag |
| | gatccATGTCATTCACTGCCAAGGAAGTTCAATATTTGCGTTCT |
| | CAGCAATTAGGAAGATTAGCCACCGTTGGTGCTGATGGTACA |
| | CCACATAATGTTCCAGTAGGCTACAGATACAACGCTGACTTG |
| | GGGACCATTGACATCACAGGAAGGGACTTAAGAAGAAGTAG |
| | GAAATATAGAGATGTTCAGGCTGGCTCGCGGGTGGCATTTAT |
| | TGTTGATGATCTACCGAGCACAGCACCTATAGTCGCCCGCGG |
| | GCTGGAGGTGAGGGGAACAGCTGAGGCGCTTTCTGGTGAAG |
| | AAGATTTGATACGTGTACGACCTGCAAGAATCGTCACTTGGG |
| | GTATTGAAGCAGATTGGCAAGCTGGTCCTACTGGTAGAACGG |
| | TAAGGCCCACCACTCCAAGATCCGCGACGATGTTAGTAGCCG |
| | GTGGTGGACCAACAGGGTTATGGTTGGCTGGTGAACTAAGGT |
| | TGGCCGGGGTTAGACCTCTTGTTTTAGAGCGTAGGACAGAAC |
| | CCTTTCCACATAGCAAAGCACTGGGTATCCATCCAAGAACTA |
| | TTGAAATGTTGGAGTTGAGAGGCCTTGCTGGTAGATTTTTAG |
| | ATGGCGCCCCGAAGCTGCCGAAAGGTCACTTTGCATCGCTTC |
| | CTGTGCCCTTAGACTATGGTGCAATGGATACTCGACATCCGT |
| | ACGGTGTCTACCGTAAACAAGTGGAAACGGAAGCTTTATTAG |
| | CCGGCCACGCAACCAGATTAGGAGTTCCAGTCCGCAGAGGT |
| | CATGAACTTGTAGGCCTAAGACAGCACGATGATGCTGTAGTC |
| | GGTACTGTTCAAGGTCCTCAAGGTAGATACGAAGTCAGAGCC |
| | GCTTATCTTGTTGGTTGTGATGGAGGAGGTTCTACCACAAGA |
| | CGCCTGGCTGGGATAGATTTCCCTGGACAAGATCCTGGTGTT |
| | GCCGTTTTGATGGCTGACGCAAGATTCCGGGACCCATTACCC |
| | TCTGATCCAGCTATGGGTCCATTCAGACGCTATGGAGTCCTG |
| | AGACCTGATTTAAGGGCATGGTTTAGTGCTATTCCTTTACCA |
| | GAAGAACAGGGTTTATGCAGAGTTATGGTTTTTTGGTATGGA |
| | CGACAATTTGAAGATAGACGTGCTCCAGTAACTGAGGATGA |
| | AATGAGAGCAGCACTTGTTGATCTAGCGGGAACTGACTTTGG |
| | TATGTACGATGTTCAATGGTTGACCAGATTCACAGACGCTTC |
| | CAGGCAAGCTGCAAGGTATAGATCTGGAAGGGTTTTCTTAGC |
| | TGGAGATGCTGCTCATACTGTCTTCCCCACTGGTGGCCCAGG |
| | TTTGAATGCAGGTTTACAAGATGCCATGAATTTAGGTTGGAA |
| | GTTAGCTGGAGCTGTTCATGGCTGGGGTGGTGCTTTACTAGA |
| | TTCATATCATGATGAGCGTTACCCTGTAGCTGAACAAGTTTT |
| | GCGTGACACAAGAACACAATGTCTATTGTTAGATCCAGACCC |
| | TCGCTTTGTTCCATTAAGAGAGACGTTAGCAGAACTATTGAG |
| | ACTAGAACCCGTAAACAGATATTTCACCGGACACAACACGGC |
| | TCTAGCAGTTAGATACGATTTGGGTGGTGCCCATCCTGCAACG |
| | GGAGGCAGAATGCCAGATGTCTTGGTCGCAACTGGTGCTGGT |
| | CAAAGAAGGTTTTCAGATTGTTGTGTTACCGGGAGAGGGGTT |
| | CTTTTAGTCACTTCTGGTGCAGATAGGGGGGAACTGTTACGAG |
| | GATGGAAAGATAGAGTGGATGTGGTAACAGGTACCGTGGCAG |
| | GACCTCTTGATGCAGCTGATCATTTAGTTAGACCTGATGGTTA |
| | TGTTGCATGGGCCGGTGGAGCTGGACACGATGAACCTGGTTT |
| | AGAAACTGCTCTTAGATTATGGTTTGGTGAACCAGTTAGTgtcga |
| | catggaacagaagttgatttccgaagaagacctcgagtaagcttggtaccgcggctagctaagatccgc |
| | tctaaccgaaaaggaaggagttagacaacctgaagtctaggtccctatttatttttttatagttatgttagtatt |
| | aagaacgttatttatatttcaaatttttcttttttttctgtacagacgcgtgtacgcatgtaacattatactgaaaa |
| | ccttgcttgagaaggttttgggacgctcgaagatccagctgcattaatgaatcggccaacgcgcgggga |
| | gaggcggtttgcgtattgggcgctcttccgcttcctcgctcactgactcgctgcgctcgg (SEQ ID |
| | NO: 34). The capitalized sequence is SEQ ID NO: 56. |
| pPGK1-dacO1-dacO4- | tggaatggcgggaaagggtttagtaccacatgctatgatgcccactgtgatctccagagcaaagttcg |
| tCYC1 inpSP-G1 | ttcgatcgtactgttactctctctctttcaaacagaattgtccgaatcgtgtgacaacaacagcctgttctc |
| | acacactcttttcttctaaccaagggggtggtttagtttagtagaacctcgtgaaacttacatttacatatat |
| | ataaacttgcataaattggtcaatgcaagaaatacatatttggtcttttctaattcgtagtttttcaagttctta |
| | gatgctttctttttctcttttttacagatcatcaaggaagtaattatctactttttacaacaaatataaaacaag |
| | gatccATGTTAGTAGCCGGTGGTGGACCAACAGGGTTATGGTTG |
| | GCTGGTGAACTAAGGTTGGCCGGGGTTAGACCTCTTGTTTTA |
| | GAGCGTAGGACAGAACCCTTTCCACATAGCAAAGCACTGGG |
| | TATCCATCCAAGAACTATTGAAATGTTGGAGTTGAGAGGCCT |
| | TGCTGGTAGATTTTTAGATGGCGCCCCGAAGCTGCCGAAAGG |
| | TCACTTTGCATCGCTTCCTGTGCCCTTAGACTATGGTGCAATG |
| | GATACTCGACATCCGTACGGTGTCTACCGTAAACAAGTGGAA |
| | ACGGAAGCTTTATTAGCCGGCCACGCAACCAGATTAGGAGTT |
| | CCAGTCCGCAGAGGTCATGAACTTGTAGGCCTAAGACAGCA |
| | CGATGATGCTGTAGTCGGTACTGTTCAAGGTCCTCAAGGTAG |
| | ATACGAAGTCAGAGCCGCTTATCTTGTTGGTTGTGATGGAGG |
| | AGGTTCTACCACAAGACGCCTGGCTGGGATAGATTTCCCTGG |
| | ACAAGATCCTGGTGTTGCCGTTTTGATGGCTGACGCAAGATT |
| | CCGGGACCCATTACCCTCTGATCCAGCTATGGGTCCATTCAG |
| | ACGCTATGGAGTCCTGAGACCTGATTTAAGGGCATGGTTTAG |
| | TGCTATTCCTTTACCAGAAGAACAGGGTTTATGCAGAGTTAT |
| | GGTTTTTTGGTATGGACGACAATTTGAAGATAGACGTGCTCC |
| | AGTAACTGAGGATGAAATGAGAGCAGCACTTGTTGATCTAG |
| | CGGGAACTGACTTTGGTATGTACGATGTTCAATGGTTGACCA |
| | GATTCACAGACGCTTCCAGGCAAGCTGCAAGGTATAGATCTG |
| | GAAGGGTTTTCTTAGCTGGAGATGCTGCTCATACTGTCTTCCC |
| | CACTGGTGGCCCAGGTTTGAATGCAGGTTTACAAGATGCCAT |
| | GAATTTAGGTTGGAAGTTAGCTGGAGCTGTTCATGGCTGGGG |
| | TGGTGCTTTACTAGATTCATATCATGATGAGCGTTACCCTGTA |
| | GCTGAACAAGTTTTGCGTGACACAAGAACACAATGTCTATTG |
| | TTAGATCCAGACCCTCGCTTTGTTCCATTAAGAGAGACGTTA |
| | GCAGAACTATTGAGACTAGAACCCGTAAACAGATATTTCACC |
| | GGACACAACACGGCTCTAGCAGTTAGATACGATTTGGGTGGT |
| | GCCCATCCTGCAACGGGAGGCAGAATGCCAGATGTCTTGGTC |
| | GCAACTGGTGCTGGTCAAAGAAGGTTTTCAGATTGTTGTGTT |
| | ACCGGGAGAGGGGTTCTTTTAGTCACTTCTGGTGCAGATAGG |
| | GGGGAACTGTTACGAGGATGGAAAGATAGAGTGGATGTGGT |
| | AACAGGTACCGTGGCAGGACCTCTTGATGCAGCTGATCATTT |
| | AGTTAGACCTGATGGTTATGTTGCATGGGCCGGTGGAGCTGG |
| | ACACGATGAACCTGGTTTAGAAACTGCTCTTAGATTATGGTTT |
| | GGTGAACCAGTTAGTATGTCATTCACTGCCAAGGAAGTTCAA |
| | TATTTGCGTTCTCAGCAATTAGGAAGATTAGCCACCGTTGGTG |
| | CTGATGGTACACCACATAATGTTCCAGTAGGCTACAGATACA |
| | ACGCTGACTTGGGGACCATTGACATCACAGGAAGGGACTTAA |
| | GAAGAAGTAGGAAATATAGAGATGTTCAGGCTGGCTCGCGGG |
| | TGGCATTTATTGTTGATGATCTACCGAGCACAGCACCTATAGT |
| | CGCCCGCGGGCTGGAGGTGAGGGGAACAGCTGAGGCGCTTTC |
| | TGGTGAAGAAGATTTGATACGTGTACGACCTGCAAGAATCGT |
| | CACTTGGGGTATTGAAGCAGATTGGCAAGCTGGTCCTACTGG |
| | TAGAACGGTAAGGCCCACCACTCCAAGATCCGCGACGgtcgacat |
| | ggaacagaagttgatttccgaagaagacctcgagtaagcttggtaccgcggctagctaagatccgctct |
| | aaccgaaaaggaaggagttagacaacctgaagtctaggtccctatttatttttttatagttatgttagtattaa |
| | gaacgttatttatatttcaaatttttcttttttttctgtacagacgcgtgtacgcatgtaacattatactgaaaacc |
| | ttgcttgagaaggttttgggacgctcgaagatccagctgcattaatgaatcggccaacgcgcggggaga |
| | ggcggtttgcgtattgggcgctcttccgcttcctcgctcactgactcgctgcgctcgg (SEQ ID |
| | NO: 35). The capitalized sequence is SEQ ID NO: 57. |
| pTEF1-oxyS-dacO1-31- | tcaagtttcagtttcatttttcttgttctattacaactttttttacttcttgctcattagaaagaaagcatagcaa |
| tADH1 in pSP-G1 | tctaatctaagttttaattacaagcggccgcATGAGGTACGATGTTGTTATAGCTGG |
| | TGCAGGACCCACCGGTTTGATGTTAGCATGTGAACTTCGGCTG |
| | GCGGGTGCCAGAACTTTGGTTTTAGAAAGATTAGCCGAGCCT |
| | TTTCCACATAGCAAAGCACTGGGTATCCATCCAAGAACTATTG |
| | AAATGTTGGAGTTGAGAGGCCTTGCTGGTAGATTTTTAGATGG |
| | CGCCCCGAAGCTGCCGAAAGGTCACTTTGCATCGCTTCCTGTG |
| | CCCTTAGACTATGGTGCAATGGATACTCGACATCCGTACGGTG |
| | TCTACCGTAAACAAGTGGAAACGGAAGCTTTATTAGCCGGCC |
| | ACGCAACCAGATTAGGAGTTCCAGTCCGCAGAGGTCATGAAC |
| | TTGTAGGCCTAAGACAGCACGATGATGCTGTAGTCGGTACTG |
| | TTCAAGGTCCTCAAGGTAGATACGAAGTCAGAGCCGCTTATC |
| | TTGTTGGTTGTGATGGAGGAGGTTCTACCACAAGACGCCTGG |
| | CTGGGATAGATTTCCCTGGACAAGATCCTGGTGTTGCCGTTTT |
| | GATGGCTGACGCAAGATTCCGGGACCCATTACCCTCTGATCC |
| | AGCTATGGGTCCATTCAGACGCTATGGAGTCCTGAGACCTGA |
| | TTTAAGGGCATGGTTTAGTGCTATTCCTTTACCAGAAGAACAG |
| | GGTTTATGCAGAGTTATGGTTTTTTGGTATGGACGACAATTTG |
| | AAGATAGACGTGCTCCAGTAACTGAGGATGAAATGAGAGCAG |
| | CACTTGTTGATCTAGCGGGAACTGACTTTGGTATGTACGATGT |
| | TCAATGGTTGACCAGATTCACAGACGCTTCCAGGCAAGCTGC |
| | AAGGTATAGATCTGGAAGGGTTTTCTTAGCTGGAGATGCTGCT |
| | CATACTGTCTTCCCCACTGGTGGCCCAGGTTTGAATGCAGGTT |
| | TACAAGATGCCATGAATTTAGGTTGGAAGTTAGCTGGAGCTG |
| | TTCATGGCTGGGGTGGTGCTTTACTAGATTCATATCATGATGA |
| | GCGTTACCCTGTAGCTGAACAAGTTTTGCGTGACACAAGAAC |
| | ACAATGTCTATTGTTAGATCCAGACCCTCGCTTTGTTCCATTA |
| | AGAGAGACGTTAGCAGAACTATTGAGACTAGAACCCGTAAAC |
| | AGATATTTCACCGGACACAACACGGCTCTAGCAGTTAGATAC |
| | GATTTGGGTGGTGCCCATCCTGCAACGGGAGGCAGAATGCCA |
| | GATGTCTTGGTCGCAACTGGTGCTGGTCAAAGAAGGTTTTCAG |
| | ATTGTTGTGTTACCGGGAGAGGGGTTCTTTTAGTCACTTCTGG |
| | TGCAGATAGGGGGGAACTGTTACGAGGATGGAAAGATAGAG |
| | TGGATGTGGTAACAGGTACCGTGGCAGGACCTCTTGATGCAG |
| | CTGATCATTTAGTTAGACCTGATGGTTATGTTGCATGGGCCGG |
| | TGGAGCTGGACACGATGAACCTGGTTTAGAAACTGCTCTTAG |
| | ATTATGGTTTGGTGAACCAGTTAGTgtatcgatggattacaaggatgacgacg |
| | ataagatctgagctcttaattaacaattcttcgccagaggtttggtcaagtctccaatcaaggttgtcggct |
| | (SEQ ID NO: 36). The capitalized sequence is SEQ ID NO: 58. |
| pTEF1-oxyS-dacO1-87- | tcaagtttcagtttcatttttcttgttctattacaactttttttacttcttgctcattagaaagaaagcatagcaa |
| tADH1 in pSP-G1 | tctaatctaagttttaattacaagcggccgcATGAGGTACGATGTTGTTATAGCT |
| | GGTGCAGGACCCACCGGTTTGATGTTAGCATGTGAACTTCGG |
| | CTGGCGGGTGCCAGAACTTTGGTTTTAGAAAGATTAGCCGAG |
| | CCTGTTGACTTCTCGAAAGCTCTAGGAGTCCACGCTCGCACT |
| | GTTGAACTATTAGATATGAGAGGCCTCGGTGAAGGATTCCAG |
| | GCTGAAGCACCAAAGTTAAGAGGTGGTAATTTTGCCTCATTA |
| | GGCGTCCCCCTGGATTTCTCATCATTTGATACTCGACATCCGT |
| | ACGGTGTCTACCGTAAACAAGTGGAAACGGAAGCTTTATTAG |
| | CCGGCCACGCAACCAGATTAGGAGTTCCAGTCCGCAGAGGT |
| | CATGAACTTGTAGGCCTAAGACAGCACGATGATGCTGTAGTC |
| | GGTACTGTTCAAGGTCCTCAAGGTAGATACGAAGTCAGAGCC |
| | GCTTATCTTGTTGGTTGTGATGGAGGAGGTTCTACCACAAGA |
| | CGCCTGGCTGGGATAGATTTCCCTGGACAAGATCCTGGTGTT |
| | GCCGTTTTGATGGCTGACGCAAGATTCCGGGACCCATTACCC |
| | TCTGATCCAGCTATGGGTCCATTCAGACGCTATGGAGTCCTG |
| | AGACCTGATTTAAGGGCATGGTTTAGTGCTATTCCTTTACCA |
| | GAAGAACAGGGTTTATGCAGAGTTATGGTTTTTTGGTATGGA |
| | CGACAATTTGAAGATAGACGTGCTCCAGTAACTGAGGATGA |
| | AATGAGAGCAGCACTTGTTGATCTAGCGGGAACTGACTTTGG |
| | TATGTACGATGTTCAATGGTTGACCAGATTCACAGACGCTTC |
| | CAGGCAAGCTGCAAGGTATAGATCTGGAAGGGTTTTCTTAGC |
| | TGGAGATGCTGCTCATACTGTCTTCCCCACTGGTGGCCCAGG |
| | TTTGAATGCAGGTTTACAAGATGCCATGAATTTAGGTTGGAA |
| | GTTAGCTGGAGCTGTTCATGGCTGGGGTGGTGCTTTACTAGA |
| | TTCATATCATGATGAGCGTTACCCTGTAGCTGAACAAGTTTT |
| | GCGTGACACAAGAACACAATGTCTATTGTTAGATCCAGACCC |
| | TCGCTTTGTTCCATTAAGAGAGACGTTAGCAGAACTATTGAG |
| | ACTAGAACCCGTAAACAGATATTTCACCGGACACAACACGG |
| | CTCTAGCAGTTAGATACGATTTGGGTGGTGCCCATCCTGCAA |
| | CGGGAGGCAGAATGCCAGATGTCTTGGTCGCAACTGGTGCTG |
| | GTCAAAGAAGGTTTTCAGATTGTTGTGTTACCGGGAGAGGGG |
| | TTCTTTTAGTCACTTCTGGTGCAGATAGGGGGGAACTGTTAC |
| | GAGGATGGAAAGATAGAGTGGATGTGGTAACAGGTACCGTG |
| | GCAGGACCTCTTGATGCAGCTGATCATTTAGTTAGACCTGAT |
| | GGTTATGTTGCATGGGCCGGTGGAGCTGGACACGATGAACCT |
| | GGTTTAGAAACTGCTCTTAGATTATGGTTTGGTGAACCAGTTA |
| | GTgtatcgatggattacaaggatgacgacgataagatctgagctcttaattaacaattcttcgccagag |
| | gtttggtcaagtctccaatcaaggttgtcggct (SEQ ID NO: 37). The capitalized |
| | sequence is SEQ ID NO: 60. |
| pTEF1-oxyS-dacO1- | tcaagtttcagtttcatttttcttgttctattacaactttttttacttcttgctcattagaaagaaagcatagcaa |
| 191-tADHlinpSP-G1 | tctaatctaagttttaattacaagcggccgcATGAGGTACGATGTTGTTATAGCT |
| | GGTGCAGGACCCACCGGTTTGATGTTAGCATGTGAACTTCGG |
| | CTGGCGGGTGCCAGAACTTTGGTTTTAGAAAGATTAGCCGAG |
| | CCTGTTGACTTCTCGAAAGCTCTAGGAGTCCACGCTCGCACT |
| | GTTGAACTATTAGATATGAGAGGCCTCGGTGAAGGATTCCAG |
| | GCTGAAGCACCAAAGTTAAGAGGTGGTAATTTTGCCTCATTA |
| | GGCGTCCCCCTGGATTTCTCATCATTTGATACTAGACACCCAT |
| | ATGCATTGTTTGTTCCACAAGTACGAACTGAAGAACTGCTAAC |
| | AGGTAGAGCTTTGGAGCTAGGGGCGGAGCTGCGTCGTGGTCA |
| | TGCCGTGACCGCCTTGGAACAAGATGCTGATGGTGTTACTGTG |
| | AGCGTGACAGGCCCTGAAGGCCCATACGAAGTAGAATGTGCT |
| | TATTTGGTGGGCTGCGACGGTGGAGGCAGTACGGTGAGGAAA |
| | CTATTGGGCATAGATTTTCCAGGTCAAGACCCACATATGTTTG |
| | CTGTCATCGCAGACGCAAGATTCAGAGAAGAGCTTCCTCACG |
| | ATCCAGCTATGGGTCCATTCAGACGCTATGGAGTCCTGAGAC |
| | CTGATTTAAGGGCATGGTTTAGTGCTATTCCTTTACCAGAAGA |
| | ACAGGGTTTATGCAGAGTTATGGTTTTTTGGTATGGACGACAA |
| | TTTGAAGATAGACGTGCTCCAGTAACTGAGGATGAAATGAGA |
| | GCAGCACTTGTTGATCTAGCGGGAACTGACTTTGGTATGTACG |
| | ATGTTCAATGGTTGACCAGATTCACAGACGCTTCCAGGCAAG |
| | CTGCAAGGTATAGATCTGGAAGGGTTTTCTTAGCTGGAGATG |
| | CTGCTCATACTGTCTTCCCCACTGGTGGCCCAGGTTTGAATGC |
| | AGGTTTACAAGATGCCATGAATTTAGGTTGGAAGTTAGCTGG |
| | AGCTGTTCATGGCTGGGGTGGTGCTTTACTAGATTCATATCAT |
| | GATGAGCGTTACCCTGTAGCTGAACAAGTTTTGCGTGACACA |
| | AGAACACAATGTCTATTGTTAGATCCAGACCCTCGCTTTGTTC |
| | CATTAAGAGAGACGTTAGCAGAACTATTGAGACTAGAACCCG |
| | TAAACAGATATTTCACCGGACACAACACGGCTCTAGCAGTTA |
| | GATACGATTTGGGTGGTGCCCATCCTGCAACGGGAGGCAGAA |
| | TGCCAGATGTCTTGGTCGCAACTGGTGCTGGTCAAAGAAGGT |
| | TTTCAGATTGTTGTGTTACCGGGAGAGGGGTTCTTTTAGTCAC |
| | TTCTGGTGCAGATAGGGGGGAACTGTTACGAGGATGGAAAGA |
| | TAGAGTGGATGTGGTAACAGGTACCGTGGCAGGACCTCTTGA |
| | TGCAGCTGATCATTTAGTTAGACCTGATGGTTATGTTGCATGG |
| | GCCGGTGGAGCTGGACACGATGAACCTGGTTTAGAAACTGCT |
| | CTTAGATTATGGTTTGGTGAACCAGTTAGTgtatcgatggattacaaggatg |
| | acgacgataagatctgagctcttaattaacaattcttcgccagaggtttggtcaagtctccaatcaaggttg |
| | tcggct (SEQ ID NO: 38). The capitalized sequence is SEQ ID NO: 61. |
| pPGK1-dacJ-tCNC1 in | tggaatggcgggaaagggtttagtaccacatgctatgatgcccactgtgatctccagagcaaagttcgtt |
| pSP-G1 (EH-5-90A) | cgatcgtactgttactctctctctttcaaacagaattgtccgaatcgtgtgacaacaacagcctgttctcaca |
| | cactcttttcttctaaccaagggggtggtttagtttagtagaacctcgtgaaacttacatttacatatatataaa |
| | cttgcataaattggtcaatgcaagaaatacatatttggtcttttctaattcgtagtttttcaagttcttagatgct |
| | ttctttttctcttttttacagatcatcaaggaagtaattatctactttttacaacaaatataaaacaaggatccAT |
| | GACCGCAGGACAGCTGCATCTTGACGTTCCGGAACCGCCTGG |
| | TTCATGGGTAGATGAAGCCACATTTAGGGCTGTTATGGGTGCT |
| | TTGCCATCCGGTGTTACAGTCGTGACGACCTTGGACCCAGATG |
| | GCAGACCAGTAGGCTTAACTTGTTCTGCGGCTTGTTCAGTTTC |
| | TCAAAGGCCTCCCTTATTACTTGTCTGCTTAAACGATAGAAGC |
| | AGGGTACTAGATGCAATACTCAGAAGAGGTCAGTTCATAGTA |
| | AATGTCCTACGCGACCGTAGAGAAGCAACATCGGCAGTCTTT |
| | GCCAGTAGAGCCGAAGATAGATTCGCAGCTGTGCCTTGGCAA |
| | CCTGGGAGGCGAACTGGTGTCCCTTGGTTGTTAGCAGACACG |
| | GTTGCTCATGCAGAATGTGAGGTGGCAGGTACTTTGCATGCTG |
| | GCGATCACGTTATCGTTGTTGGAGGTATTGTTGCGGGTGATGC |
| | GCACCCCGACCGTGTGGGGCCATTAATGTATTGGAGACAAAG |
| | ATACGGTGGATGGCCAGTAACAGATGATGCTAGAGAAATTGC |
| | ATTGACTCTAGCTGCCGAGGGAgtcgacatggaacagaagttgatttccgaagaa |
| | gacctcgagtaagcttggtaccgcggctagctaagatccgctctaaccgaaaaggaaggagttagac |
| | aacctgaagtctaggtccctatttatttttttatagttatgttagtattaagaacgttatttatatttcaaatttt |
| | tcttttttttctgtacagacgcgtgtacgcatgtaacattatactgaaaaccttgcttgagaaggttttgggacgc |
| | tcgaagatccagctgcattaatgaatcggccaacgcgcggggagaggcggtttgcgtattgggcgct |
| | cttccgcttcctcgctcactgactcgctgcgctcgg (SEQ ID NO: 39). The capitalized |
| | sequence is SEQ ID NO: 62. |
| pGPD-dacM2-tCYC1 in | ccctcactaaagggaacaaaagctggagctcAGTTTATCATTATCAATACTGCC |
| pRS413 | ATTTCAAAGAATACGTAAATAATTAATAGTAGTGATTTTCCTA |
| | ACTTTATTTAGTCAAAAAATTAGCCTTTTAATTCTGCTGTAAC |
| | CCGTACATGCCCAAAATAGGGGGCGGGTTACACAGAATATAT |
| | AACATCGTAGGTGTCTGGGTGAACAGTTTATTCCTGGCATCCA |
| | CTAAATATAATGGAGCCCGCTTTTTAAGCTGGCATCCAGAAA |
| | AAAAAAGAATCCCAGCACCAAAATATTGTTTTCTTCACCAAC |
| | CATCAGTTCATAGGTCCATTCTCTTAGCGCAACTACAGAGAAC |
| | AGGGGCACAAACAGGCAAAAAACGGGCACAACCTCAATGGA |
| | GTGATGCAACCTGCCTGGAGTAAATGATGACACAAGGCAATT |
| | GACCCACGCATGTATCTATCTCATTTTCTTACACCTTCTATTAC |
| | CTTCTGCTCTCTCTGATTTGGAAAAAGCTGAAAAAAAAGGTTG |
| | AAACCAGTTCCCTGAAATTATTCCCCTACTTGACTAATAAGTA |
| | TATAAAGACGGTAGGTATTGATTGTAATTCTGTAAATCTATTT |
| | CTTAAACTTCTTAAATTCTACTTTTATAGTTAGTCTTTTTTTTA |
| | GTTTTAAAACACCAAGAACTTAGTTTCGACGGATACTAGTAA |
| | AATGACTGATGTATCCGCCCATACTACAACCGATGGGGCGGT |
| | TGCAAGGGCTCGCGCTGTGGTCAGACTAAATACTGCATATTTT |
| | CGGGCTAAGGTACTTCAAAGTGCTGTCGAACTAGGTGTTTTTG |
| | ATCTTTTAGATGGAGGCCCACAACCTGCCGCTGACATTTGCGG |
| | AAAGTTAGGTATTAGACATAGACTCGCAGTGGACTATTTGGA |
| | CGCATTGACGGGTTTAGGGTTGCTTGAAAGGGATGGTGAACG |
| | TTACAGAAACTCGCCAACAGCGGCTGAGTTCTTGGTCCAAGA |
| | TCGACCCGCCTATTTGGGTGGAACCGCGAGGCAACATGCCAA |
| | ATTACATTATCACGCTTGGGGTAAACTGGCCGATGCAATGAG |
| | AGAAGGCCGTGCTCAGAGCGCCGTGGCAGCTCAAGGTGCAAA |
| | AGCCTATGTAAATTACTATGAAGATTTGGGCCAGGCCAGGAG |
| | GGTCATGACACATATGGATGCTCATAACGGTTTCACAGCGGA |
| | TGAAATCGCTAGACAATTGGACTGGAGAGGATACGCCACAGT |
| | AACAGATGTGGGCGGCGCTCGTGGGAATGTTGCAGCTCGAAT |
| | TGTTCAAGCAGTGCCTCATTTGAGAGCAAATGTAGTTGACCTA |
| | CCCGCATTGCGACCTTTATTCGATGAGCTGATGGCCCAACTAG |
| | GAACTGCTGGTCAGGTTGAGTTTCACGGTGGTGACTTCTTTGC |
| | GGACCCGATACCAGAGTCTGACGTTATCGTTGTGGGCCACGT |
| | CCTGGCGGATTGGCCACAGCCAAGACGTGTGGAATTACTTAG |
| | AAGATGTCACGCAGCATTAAGACCTGGTGGAACGGTAGTTGT |
| | TTACGATGTTATGGTTGATGATGATAGAGATGACGTCGAAGC |
| | ACTCCTACAAAGATTAAACTCTGCAATGATAAGGGACGATAT |
| | GGGTGCTTACGCTGTTACTGAATGTGCTGGATATTTAAGAGAA |
| | GCTGGATTTACCGTAGAAAGGGCCCTGCGTACGGATACCATA |
| | ACTCGCGATCATTTTGTTATTGGTAGAAAAGCTGCATCATAAC |
| | TCGAGtcatgtaattagttatgtcacgcttacattcacgccctccccccacatccgctctaaccgaaaa |
| | ggaaggagttagacaacctgaagtctaggtccctatttatttttttatagttatgttagtattaagaacgttattt |
| | atatttcaaatttttcttttttttctgtacagacgcgtgtacgcatgtaacattatactgaaaaccttgcttgaga |
| | aggttttgggacgctcgaaggctttaatttgcggccggtacccaat (SEQ ID NO: 40). The |
| | capitalized sequence is SEQ ID NO: 63. |

High throughput assay of hydroxylation/reduction assay in whole cells in microtiter plates. First, high throughput assay of hydroxylation/reduction assay in whole cells in microtiter plates were developed using the following procedure. Fresh colonies of strains to screen harboring a plasmid for the hydroxylation and/or reduction enzyme and control strains (strain encoding OxyS as positive control and strain with no hydroxylase as negative control) were inoculated in 200 μL selective media (U⁻ or HU⁻) in 96-well and placed in shaker overnight. Overnight cultures were used to inoculate 1 mL selective media (U⁻ or HU⁻) cultures in deep well plates (Corning P-2ML-SQ-C-S) with an average starting OD of 0.01. The plate was covered with two layers of SealMate film (Excel Scientific, SM-KIT-BS) and placed in shaker overnight. Cells were then pelleted at 4° C. at 4,000 rpm. Each pellet was redissolved in 0.3 mL containing a 150 μL solution of 5 mg/mL anhydrotetracycline HCl, 30 μL 1 M Tris buffer pH 7.45, 15 μL 40% glucose solution in H₂O and 105 μL H₂O for final concentrations of 2.5 mg/mL anhydrotetracycline HCl, 100 mM Tris pH 7.45 and 2% glucose. The plates were then covered with two layers of SealMate film (Excel Scientific, SM-KIT-BS) and placed in shaker overnight at 800 rpm. 2 μL of Overnight suspensions were diluted into 198 μL of H₂O before UV/VIS spectroscopic measurements. UV/VIS spectroscopic measurements taken were as follows: (i) absorption spectrum 350 nm-500 nm, (ii) emission spectrum 450 nm-550 nm (λ_{excitation}=400 nm), (iii) excitation spectrum 305 nm-455 nm (λₑₘᵢₛₛᵢₒₙ=500 nm). Spectral steps in the spectrum were 25 nm.

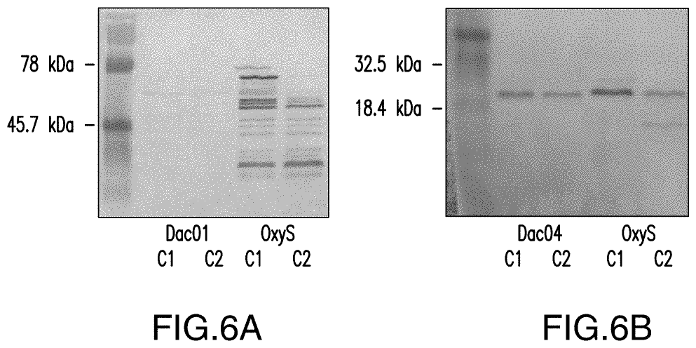

Western blots. Fresh patches or colonies of strains harboring the plasmid for the hydroxylation and/or reduction enzyme and control strains were inoculated in 3 mL selective media (U- or H) in 15 mL culture tubes (Corning 352059) and placed in shaker overnight. Overnight cultures were used to re-inoculate 3 mL selective media (U- or H) in 15 mL culture tubes (Corning 352059) and placed in shaker overnight with a starting OD of 0.01-0.05 (all western Blots shown in this study except for western blots shown in FIG. 6 which were pelleted and lysed immediately). Cells were grown to final OD of 0.6-0.8 before pelleting in 2 separate eppendorf tubes (1 mL culture each) at 4 oc at 14,000 rpm, removing the supernatant and freezing at −20° C. prior to further use. 100 μL of a 99:1 mixture of Y-PER yeast protein extraction reagent (ThermoFisher Scientific 78991) and HALT protease inhibitor cocktail (ThermoFisher Scientific PI87786) were added to the tubes. The tubes were placed on orbital shaker for 20 min at r.t., followed by 10 min centrifugation at 14,000 rpm at 4 oc and the cell lysate was transferred to a new 1.5 mL Eppendorf tube and kept on ice. Following 5 min at 95° C. with SDS loading buffer, an SDS-page was run and the gels were transferred to PVDF blots (Thermo Fisher Scientific IB24001) and western blots were performed according to manufacturer guidelines (Thermo Fisher R951-25 and Sigma-Aldrich A8592 for myc- and FLAG-tag, respectively) with BSA used instead of milk and analyzed with Thermo Scientific 34000 substrate kit for horseradish peroxidase.

Optimizing DacO1 expression in S. cerevisiae. In the present Example, N-terminal fusions proteins were used. Three fusion proteins composed of an N-terminal portion of OxyS and a C-terminal portion of DacO1 were tested. These fusion proteins include the first 37, 87 and 191 amino acids of OxyS, followed by the last 461, 411 and 307 amino acids of DacO1, respectively. In order to determine where to fuse the proteins, this Example considered the structure of OxyS as determined previously by X-ray crystallography, and made fusions proteins in disordered regions of the structure around positions 37, 87 and 191.

For inducible promoters, several promoters were tested: pGAL1, induced by galactose, and pADH2, a late stage promoter induced in low glucose/high EtOH. The alternative strain background chosen was BJ5464-NpgA. For a lower temperature, culturing was attempted at 25° C.

In order to provide DacO1 with enzymes potentially required to form a functional oligomeric complex the co-expression of DacJ and DacM2 was tested. DacJ and DacM2 were chosen since they are the closest enzymes to DacO1 in the dactylocycline gene cluster and they are proposed to share with DacO1 the functional role of aglycone tailoring.

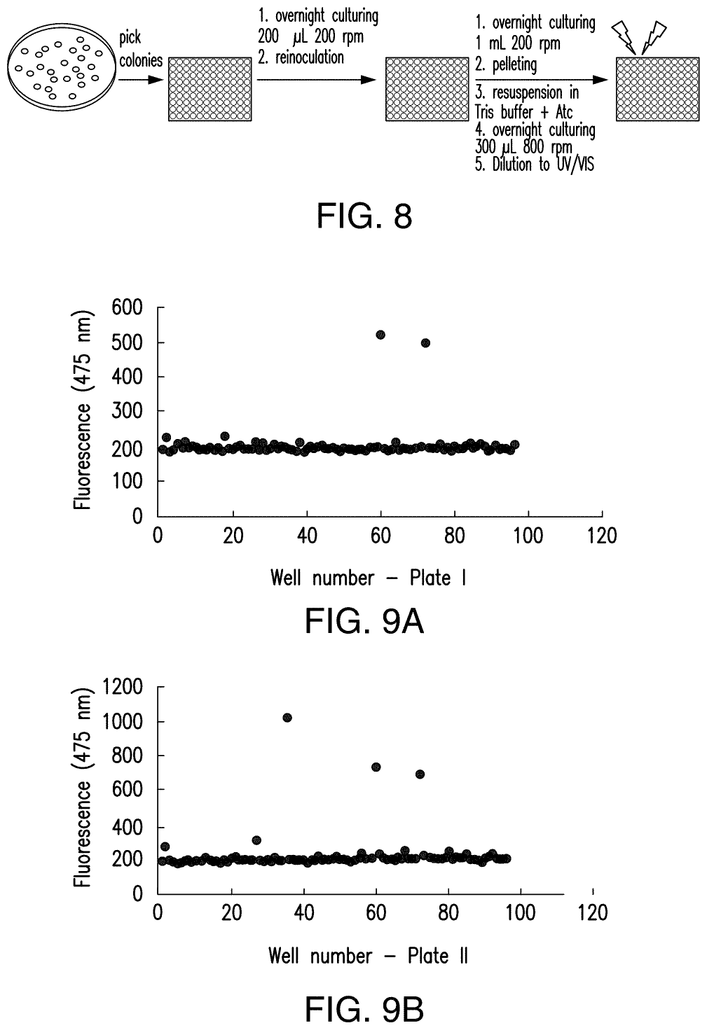

Testing DacO1 expression optimization constructs and other bacterial hydroxylases. Strains generated for the DacO1 optimization attempts as well as their controls, EH-5-98-1 through EH-5-98-19, EH-3-248-1 through EH-3-248-8, EH-3-80-2, EH-3-80-3, EH-3-986 and EH-5-115-1 through EH-5-115-5, were screened using the microtiter plate UV/Vis assay. Each of the 35 strains was assayed in four biological replicates, as well as in two culturing temperatures, 30° c. and 25° c., starting from the 2^{nd} inoculation stage (FIG. 8). The protocol for the microtiter plate UV/Vis assay was slightly modified to accommodate the strains encoding DacO1 controlled by inducible promoters. EH-5-98-7 and EH-5-98-8 encoding pADH2-dacO1 were inoculated in YPD in the 2nd inoculation and EH-5-98-4 and EH-5-98-5, encoding pGAL1-dacO1 were inoculated in U⁻ Raffinose and supplemented in the next morning with 66.7 μL of 30% galactose in H₂O for a total concentration of 2%. UV/Vis measurements were taken following one night and after three nights of incubation with anhydrotetracycline for the 30° c. plates and after one night only for the 25° C. plates.

Strains that showed a blue shift in their peak in the Δexcitation spectrum from that of negative control strain EH-3-80-2 harboring empty pSP-G1 were indicated as potential hits in the assay and are shown in Table 3. Specifically, strains listed in the table have shown in the Δexcitation spectrum a peak of 405 nm, 380 nm or 355 nm as opposed to the 430 nm peak of negative control EH-3-80-3, associated with anhydrotetracycline. Results from three measurements are shown, two measurements after overnight incubation of the cells suspensions in the buffer containing anhydrotetracycline, one for each of the culturing temperatures (25° C. and 30° C.) and another final measurement for the 30° C. culturing condition after 3 nights of suspension in the buffer containing anhydrotetracycline. The rank was calculated per plate measured for one of the measurements according to the emission at the peak maximum and can be interpreted only as a rough indicator as peak emission at three different wavelengths are ranked on the same scale. The number 1 or 2 after the dot indicates the plate number from which the measurement was taken.

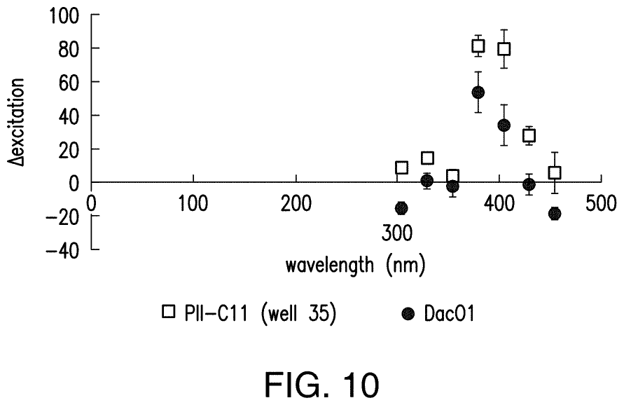

Reassuringly, the positive control OxyS, whose native substrate is anhydrotetracycline was leading the rank in highest emission resulting from a blue-shifted excitation. It was followed by the various DacO1 fusion proteins as well as by hydroxylase alternatives to OxyS and DacO1. Surprisingly, empty pSP-G1 and OxyR in the alternative background strain, BJ-5464-NpgA have also displayed a blue-shifted peak in the excitation spectrum, although they are the lowest ranking (Table 7). Also surprisingly, EH-3-80-2 encoding unoptimized DacO1 has not displayed a blue shifted excitation peak in the reduction spectrum in contrast to previous work (FIG. 10). Strains that exhibited a blue-shifted excitation peak at 25° C. and not in 30° C. are: DacO1-DacO4, OxySDacO1-191, no-AB-tags and DacO1-JCAT-BJ. I proceeded to analyze by western blot the hydroxylase expression of the strains that displayed a blue-shifted excitation peak in all three measurements.

The potential hits in the screen of DacO1 expression optimization are shown in Table 6.

TABLE 6
Potential hits in the screen of DacO1 expression optimization constructs
and other bacterial hydroxylases.

| Strain | | 30° C. | 25° C. | 30° C. | Rank 30° C. |
|---|---|---|---|---|---|
| number | Strain description | measurement I | measurement I | measurement II | measurement I |
| EH-3-98-6 | OxyS | Y | Y | Y | 1.1 |
| EH-3-98-6 | OxyS | Y | Y | Y | 1.2 |
| EH-5-98-9 | Ubiquitin-Y-IFN- | Y | Y | Y | 2.1 |
| | DacO1 | | | | |
| EH-3-248-2 | OxyS-OxyR-BJ | Y | Y | Y | 2.2 |
| EH-5-98-1 | Ubiquitin-DacO1 | Y | Y | Y | 3.1 |
| EH-3-248-1 | OxyS BJ | Y | Y | Y | 3.2 |
| EH-5-98-10 | γ-IFN-DacO1 | Y | Y | Y | 4.1 |
| EH-5-115-4 | OxyS JCAT | Y | Y | Y | 4.2 |
| EH-3-98-2 | hSOD-DacO1 | Y | Y | | 5.1 |
| EH-3-248-7 | PgaE-JCAT-BJ | Y | Y | Y | 5.2 |
| EH-5-98-3 | GalO1-DacO1 | Y | Y | Y | 6.1 |
| EH-3-248-5 | CtcN-JCAT-BJ | Y | Y | Y | 6.2 |
| EH-5-98-12 | DacO4-DacO1 | Y | Y | Y | 7.1 |
| EH-5-115-5 | DacO1-JCAT-BJ | Y | Y | Y | 7.2 |
| EH-5-98-14 | OxyS-DacO1-87 | Y | Y | Y | 8.1 |
| EH-3-248-6 | SsfO1-JCAT-BJ | Y | Y | Y | 8.2 |
| EH-5-98-13 | OxyS-DacO1-37 | Y | Y | Y | 9.1 |
| EH-3-248-4 | DacO1-BJ | Y | Y | Y | 9.2 |
| EH-3-248-3 | OxyR | Y | | | 10.2 |
| EH-3-248-8 | pSP-G1-empty-BJ | Y | Y | Y | 11.2 |
| EH-98-11 | DacO1-DacO4 | | Y | | |
| EH-5-98-15 | OxyS-DacO1-191 | | Y | | |
| EH-5-98-6 | DacO1-no-AB- | | Y | | |
| EH-5-98-16 | DacO1-JCAT-BJ | | Y | | |
| EH-5-98-5 | pGAL-DacO1- | | Y | Y | |

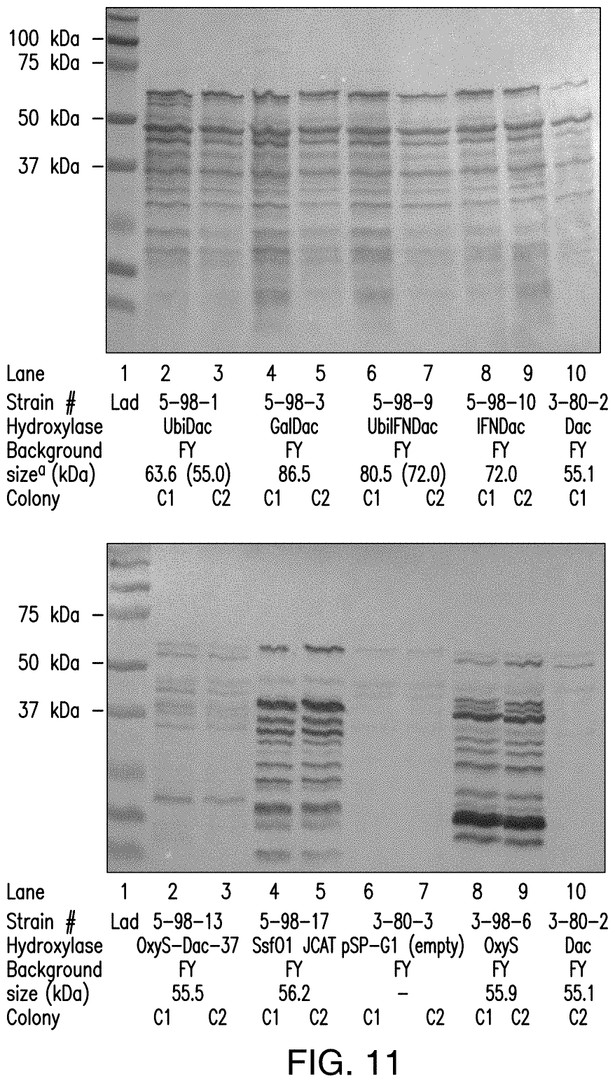

For the fusion protein constructs in FY251, ubiquitin-DacO1-C1, Gall-DacO1-C1 and Ubiquitin-IFN-DacO1-C1 slight bands are observed in the expected sizes that are not observed in the DacO1-C1 control (55.0, 86.5 and 72.0 kDa, respectively, FIG. 11). Bands at the expected sizes were also observed for OxyS-DacO1-37, SsfO1, OxyS and DacO1-C2, with SsfO1 and OxyS bands looking more prominent (55.5, 56.2, 55.9 and 55.1 kDa, respectively, FIG. 9). OxyS-DacO1-37 and DacO1-C2 show a 50<band <75 that is not observed in the empty pSP-G1 negative control. In all bands, proteins of lower mass are noticeable, potentially corresponding to proteolysis products of the hydroxylases retaining the C-terminal FLAG-tag.

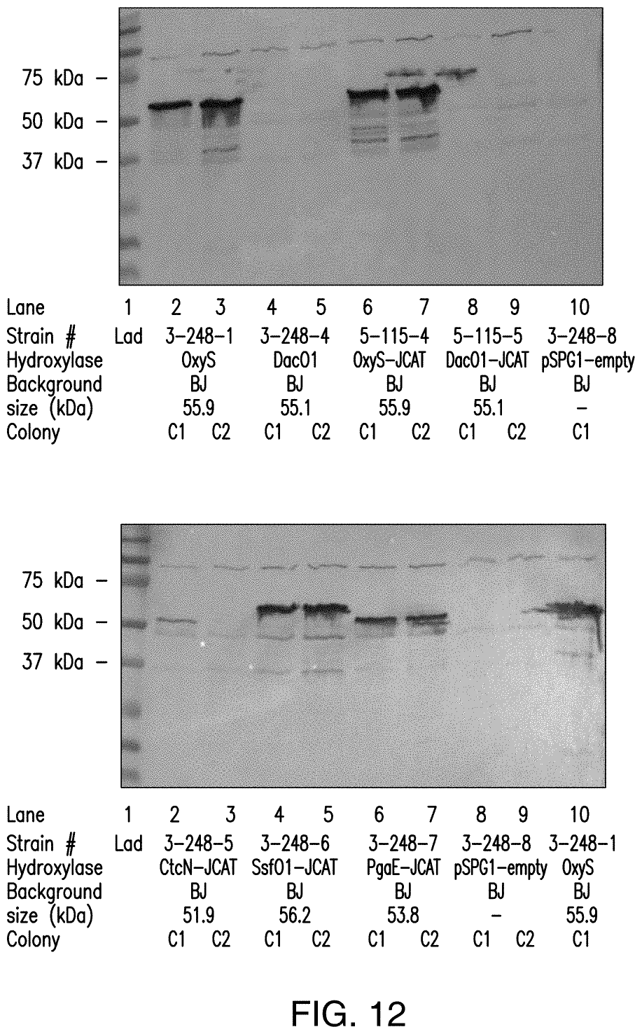

For the fusion protein constructs in BJ-5464-NpgA, clearly less degradation products are observed (FIG. 12). OxyS, SsfO1 and PgaE show very prominent bands at the expected sizes (55.9, 56.2 and 53.8 kDa) for both colonies; CtcN-C1 shows a slighter band in the expected size (51.9 kDa) and DacO1-JCAT-C2 shows an even slighter band of the expected size (55.1 kDa). In addition, the codon optimization method (COOL vs JCAT) does not seem to significantly alter expression levels in the case of OxyS, or significantly remediate expression levels in the case of DacO1 (FIG. 12). Finally, even though BJ-5464-NpgA expression apparently reduced protein degradation for other hydroxylases (e.g., OxyS and SsfO1) it does not appear to have promoted significant expression levels for DacO1 (Compare strains EH-3-248-1 and EH-3-248-4 with strains EH-5-115-4 and EH-5-115-5, respectively, FIG. 12).

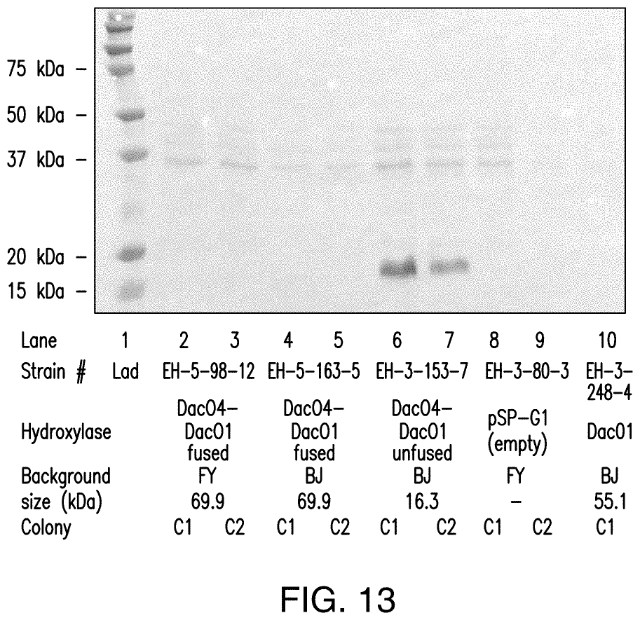

The DacO4-DacO1 fusion did not yield a stably expressed protein. While DacO4 under pPGK1 and labeled with myc-tag is clearly expressed when unfused to DacO1, the DacO4-DacO1 fusion is not observed in the gel when under pPGK1 and labeled with myc-tag, in either FY251 or BJ5464-NpgA background strain (FIG. 13).

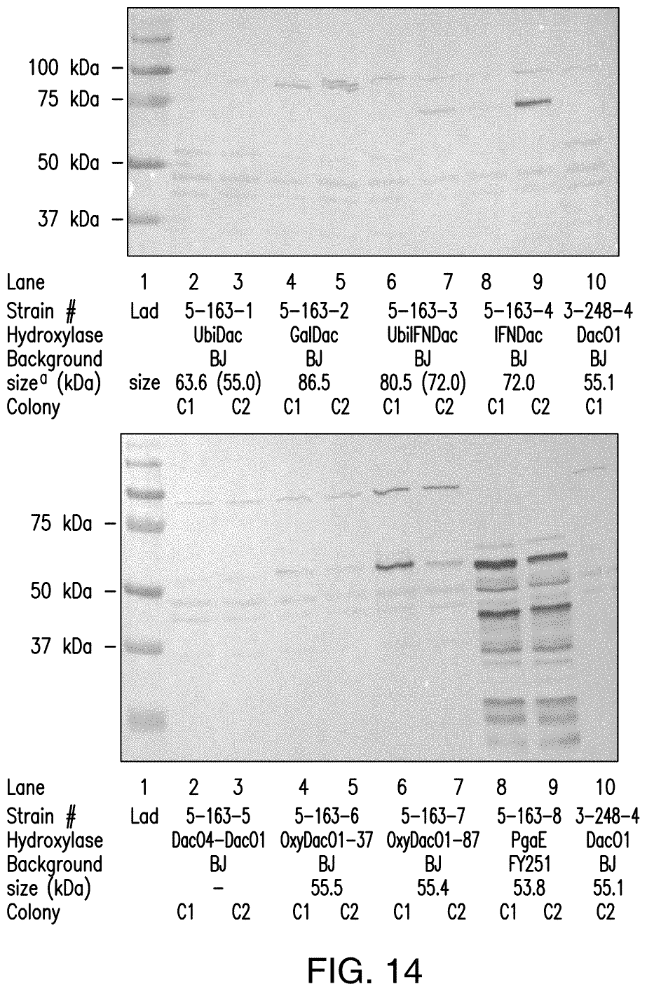

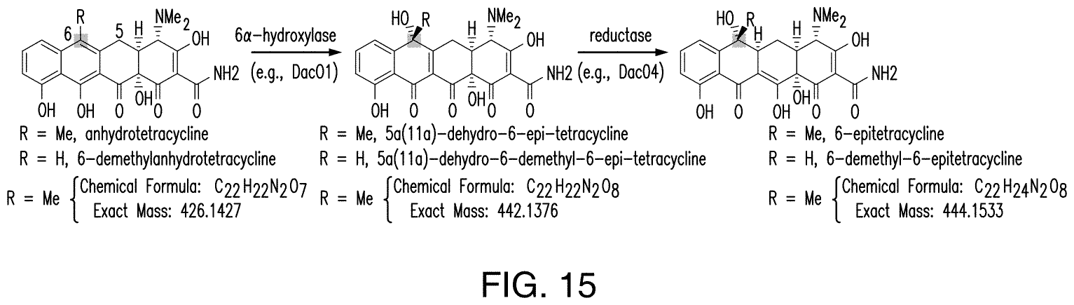

In BJ5464-NpgA background, Ubiquitin-DacO1 fusion expression levels seem similar to that of unfused DacO1; Gall-DacO1, Uniquitin-IFN-DacO1 and IFN-DacO1-C2 fusions gave bands at the expected sizes of 86.5 and 72.0, respectively, with IFN-DacO1-C2 showing the most prominent band; The OxyS N-terminal fusions to C-terminal DacO1, OxyS-DacO1-37 and OxyS-DacO1-87 both gave bands at the expected size with OxyS-DacO1-87-C1 showing the more prominent band (FIG. 14). Based on these results, the colonies showing better DacO1 expression levels were chosen for analysis of anhydrotetracycline hydroxylation by mass spectrometry (FIG. 15).

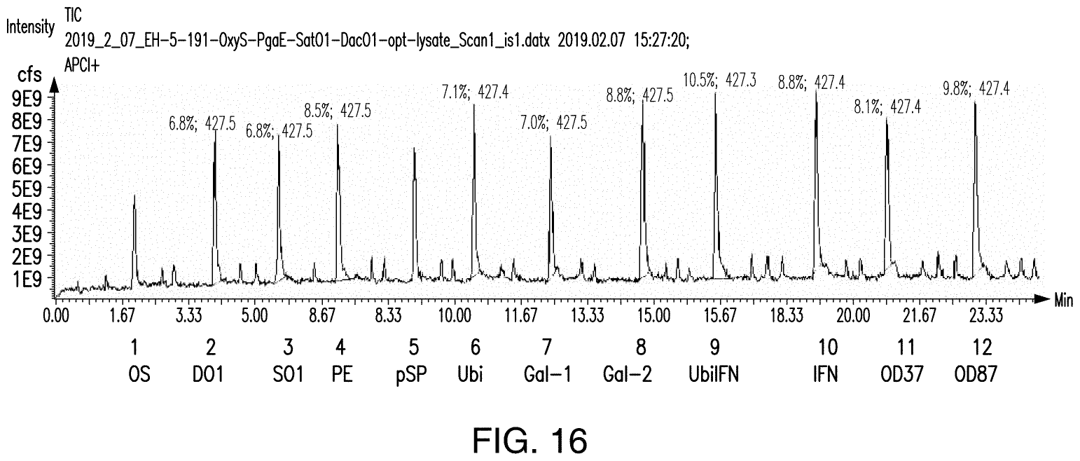

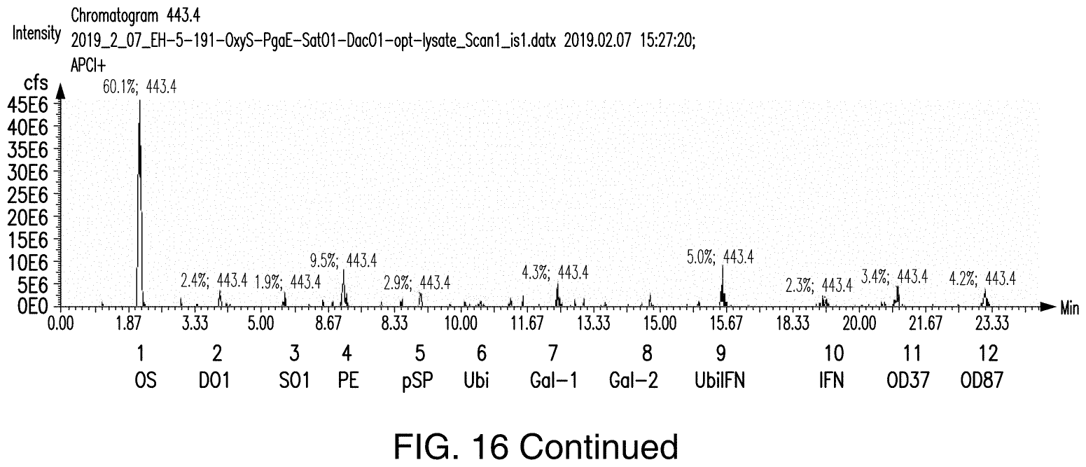

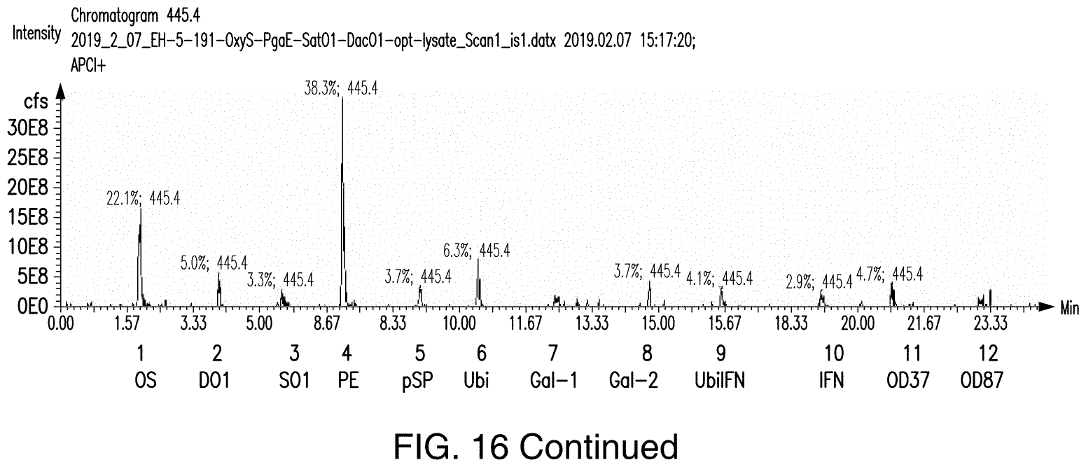

Cell lysates of strains expressing hydroxylases that showed stronger bands of the expected sizes in the Western Blots were incubated in a TRIS buffer with anhydrotetracycline and glucose-6-phosphate. The overnight incubations were then assayed by mass and UV/Vis spectrometry (FIG. 16). The UV/Vis data are shown in Table 7. The strains that show hydroxylation levels potentially above background, as indicated by 443.4 ion counts that are more than double those for the no hydroxylase negative control, are those encoding OxyS, PgaE and Ubiquitin-γ-IFN-DacO1 (labeled as 1, 4 and 9, respectively in FIG. 16). These three strains, along with three additional ones encoding γ-IFN-DacO1, OxySDac37 and OxySDac87, also show a maximal emission and/or excitation in the Aspectra that differs from that of the background strain EH-3-248-8 encoding empty pSP-G1 (Table 8). Therefore, these six strains along with EH-3-248-4 and EH-3-248-8 as controls were assayed again by mass spectrometry in a wider set of conditions prior to larger scale reactions, isolation and analysis by NMR.

TABLE 7
UV/Vis spectroscopy results for hydroxylase strains cell lysates
incubated with anhydrotetracycline and glucose-6-phosphate.

| | | | Max emission | Max excitation |
|---|---|---|---|---|
| | | | (λexcitation = | (λemission = |
| | Strain no. | Hydroxylase | 280 nm) | 500 nm) |
| 1 | EH-3-248-1 | OxyS^{b} | 490 | 390 |
| 2 | EH-3-248-4 | DacO1 | 590 | 350 |
| 3 | EH-3-248-6 | SsfO1 | 580 | 350 |
| 4 | EH-3-248-7 | PgaE^{b} | 580 | 370 |
| 5 | EH-3-248-8 | pSPG1^{a} | 580 | 350 |
| 6 | EH-5-163-1-C1 | UbiDac | 570 | 350 |
| 7 | EH-5-163-2-C1 | GalDac | 570 | 350 |
| 8 | EH-5-163-2-C2 | GalDac | 570 | 350 |
| 9 | EH-5-163-3-C2 | UbiIFNDac^{b} | 690 | 350 |
| 10 | EH-5-163-4-C2 | IFNDac^{b} | 460 | 350 |
| 11 | EH-5-163-6-C1 | OxySDac37^{b} | 420 | 370 |
| 12 | EH-5-163-7-C1 | OxySDac87^{b} | 430 | 470 |

^{a}The maximum emission and maximum absorption spectra of EH-3-248-8 encoding no hydroxylase (empty pSP-G1) are shown in italics. For all other strains, the maximum value shown are from the Δspectrum of the corresponding strain minus the spectrum of the EH-3-248-8 strain encoding no hydroxylase (empty pSP-G1).
^{b}Maximum emission and excitation values for the Aspectra that are 10 nm different or more than the corresponding values obtained for strain EH-3-248-8 encoding no hydroxylase (empty pSP-G1) are shown in bold.

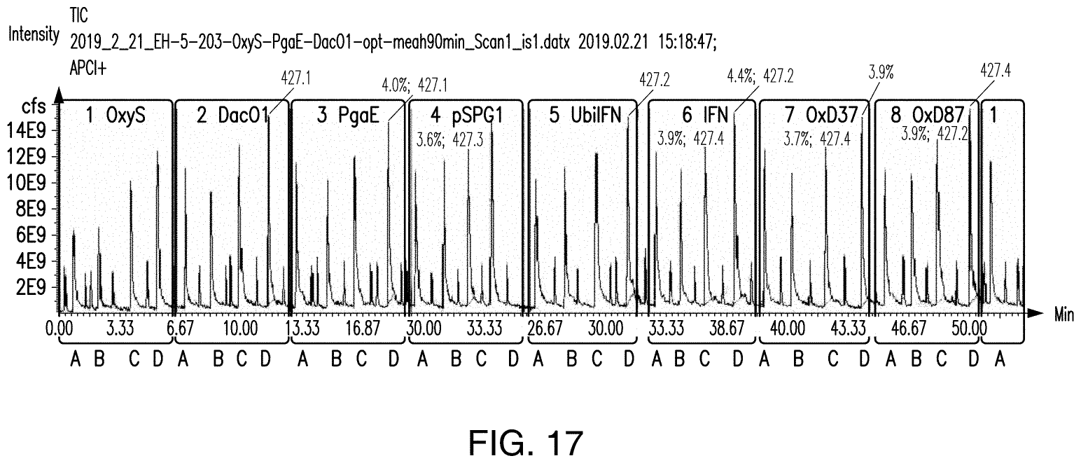

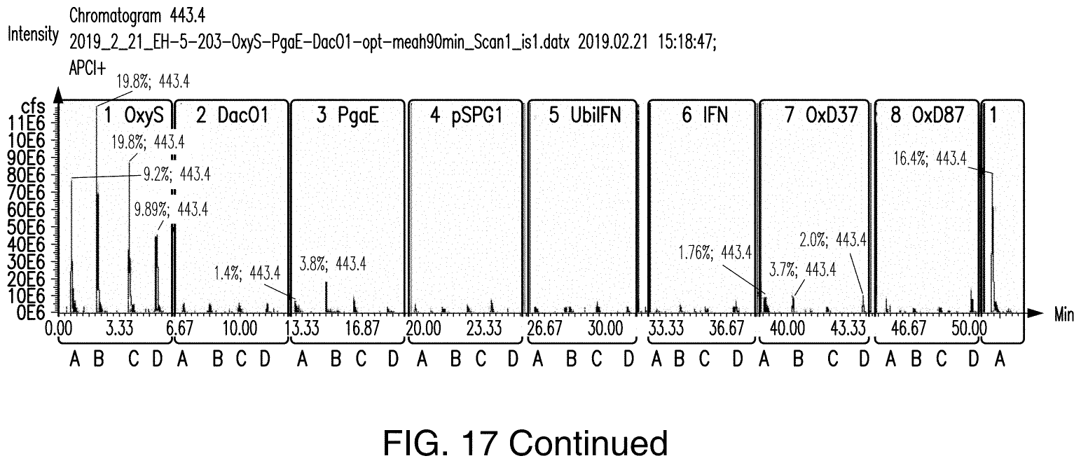

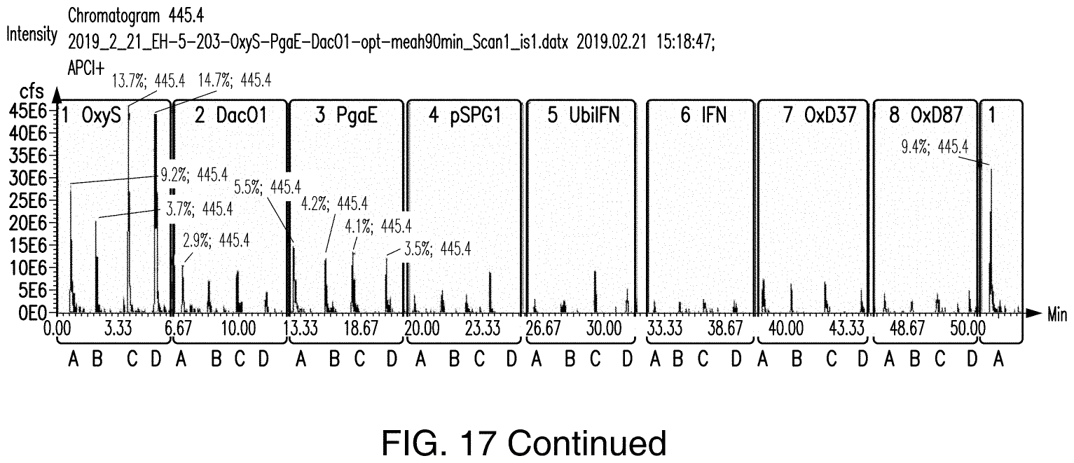

To select strains and conditions for larger scale reactions, strains encoding hydroxylases that performed favorably according to the Western Blot, mass spectrometry and UV/Vis spectroscopy results were analyzed in a larger set of conditions using mass spectrometry. Following cell lysis, lysates were incubated overnight in four different conditions (Table 8) and analyzed by mass spectrometry. The rationale behind trying different conditions was that given that the conversion rates for all non-OxyS hydroxylases were significantly lower than for OxyS (FIG. 16), the reaction can be optimized to allow enough product to be isolated for NMR analysis. The strains that show hydroxylation levels potentially above background, as indicated by 443.4 ion counts that are more than double those for the no hydroxylase negative control encoding empty pSP-G1, are OxyS and PgaE, with OxyS-DacO1-37 as the closest runner up (FIG. 17). These four strains are used for a larger scale lysate experiment followed by product isolation and analysis by NMR spectroscopy. Condition D, maximizing the m/z peak associated with tetracycline per amount of cell lysate used is used to attempt the isolation of tetracycline and condition B maximizing the m/z peak associated with 5a(11a)-dehydrotetracycline is used to attempt the isolation of the latter.

TABLE 8
Assay conditions for anhydrotetracycline hydroxylation and reduction
in cell lysates of strains expressing DacO1, its fusion proteins and other
bacterial hydroxylases.

| Condition | Atc | G6P | Cell lysate dilution factor |
|---|---|---|---|
| no.^{a} | (mM) | (10 mM) | into reaction mix |
| A | 5.4 | — | 5× |
| B | 5.4 | — | 5× |
| C | 21.6 | — | 5× |
| D | 21.6 | — | 10× |

^{a}Cell lysates were placed overnight in TRIS buffer (100 mM, pH 7.45) containing in addition to anhydrotetracycline and glucose-6-phosphate at the concentrations mentioned above, glucose (27.8 mM), NADPH (3 mM) and mercaptoethanol (18.5 mM).

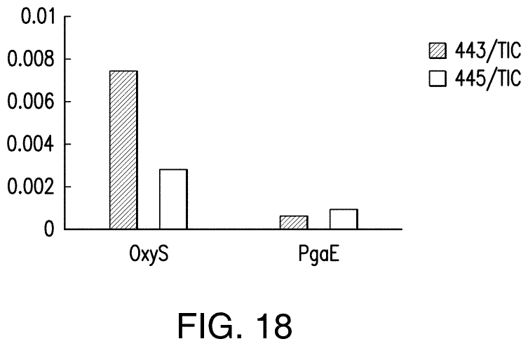

While for both OxyS and PgaE, in the presence of G6P there is an increase in 445 ion counts associated with the product of both hydroxylation and reaction (FIG. 5 and FIG. 17), the PgaE samples were consistently associated with higher 445/443 ion count ratios relative to the OxyS samples in the presence of G6P (FIG. 18). This supports that in the case of PgaE hydroxylation a larger fraction of the hydroxylation product ([M−H]⁺=443) gets further reduced ([M−H]⁺=445).

The present Example shows development of a stably expressing form of DacO1 in S. cerevisiae (FIG. 14), the identification of PgaE as an anhydrotetracycline hydroxylase (FIG. 16 and FIG. 17), and the discovery that a larger proportion of PgaE hydroxylated intermediates undergo reduction in the presence of glucose-6-phosphate than is the case in OxyS (FIG. 18).

### Example 3. The biosynthesis of 6-methyl-6-epitracyclines from TAN-1612 in S. cerevisiae

The present Example provides for biosynthesis of 6-methyl-6-epitracyclines from TAN-1612 in S. cerevisiae. Same protocols for high throughput assay of hydroxylation/reduction assay in whole cells in microtiter plates and for western blots as described in Example 2 were followed.

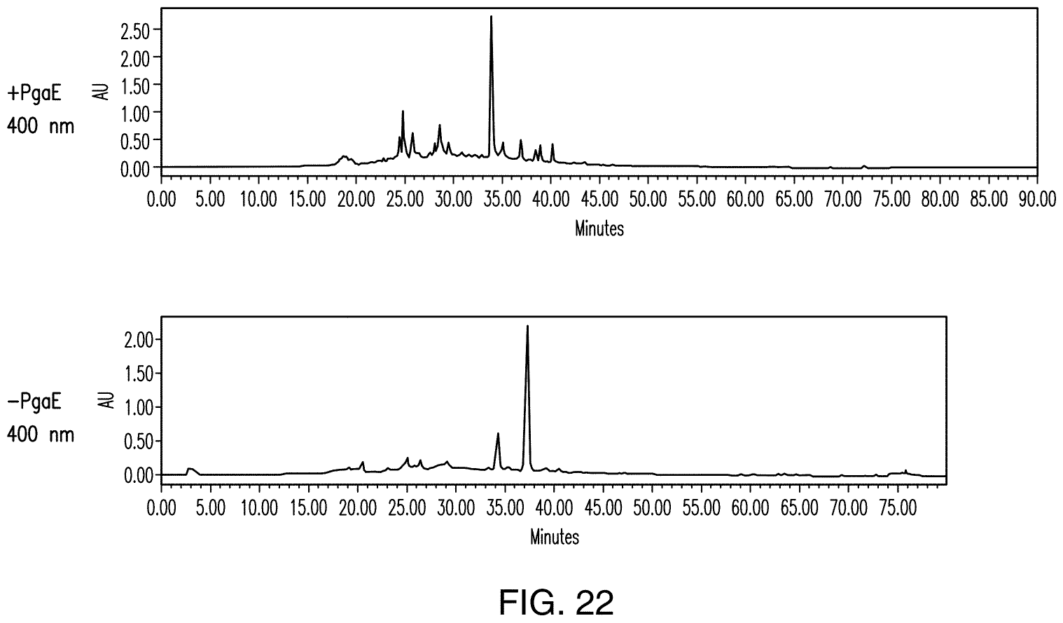

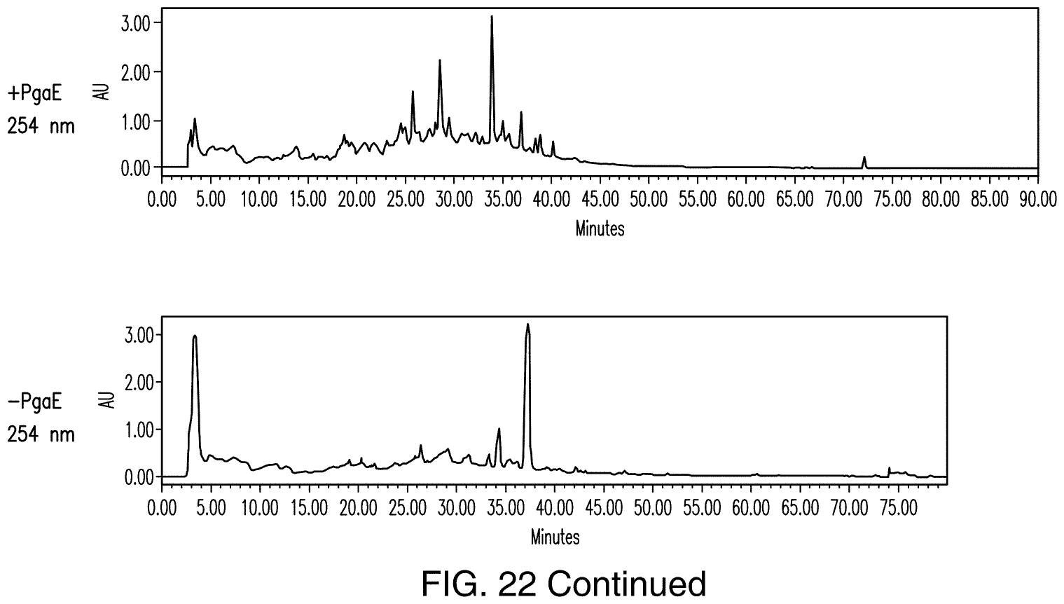

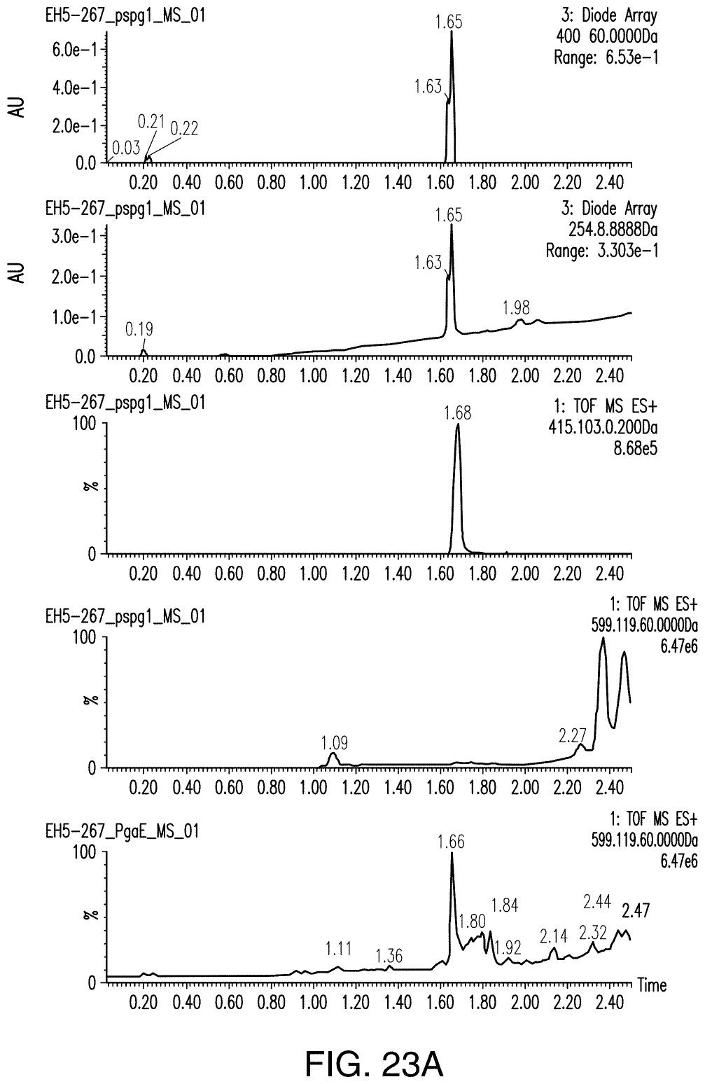

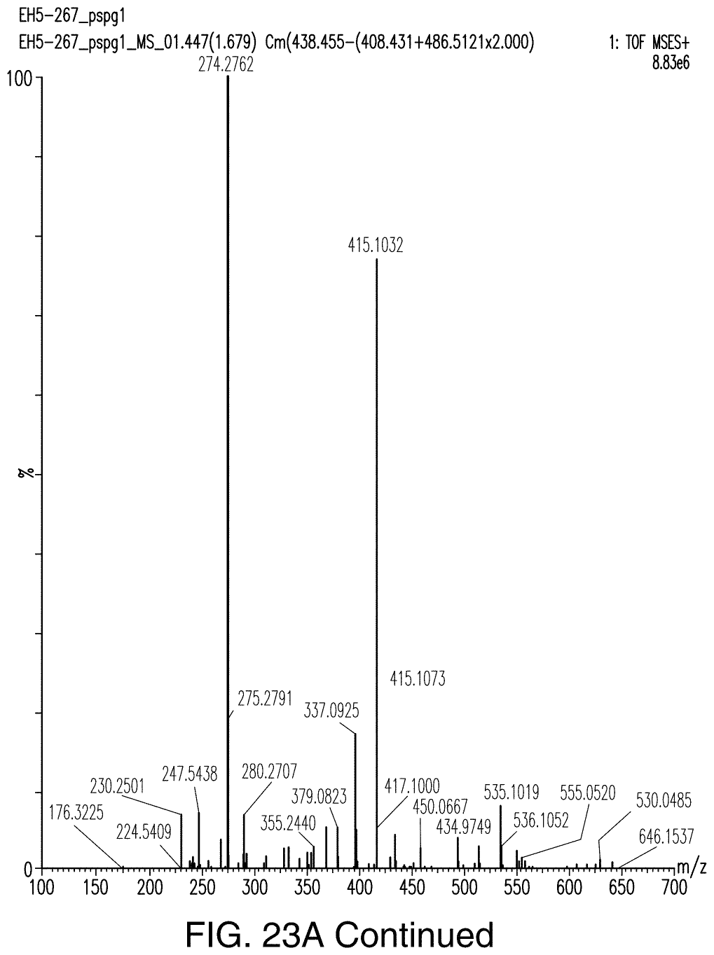

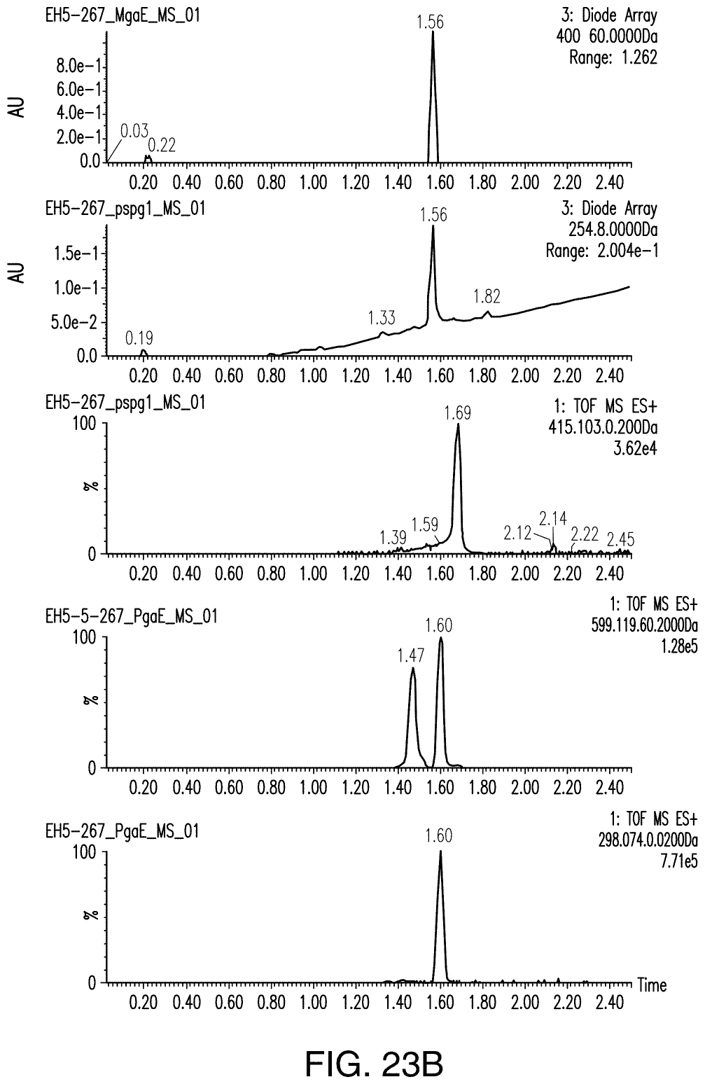

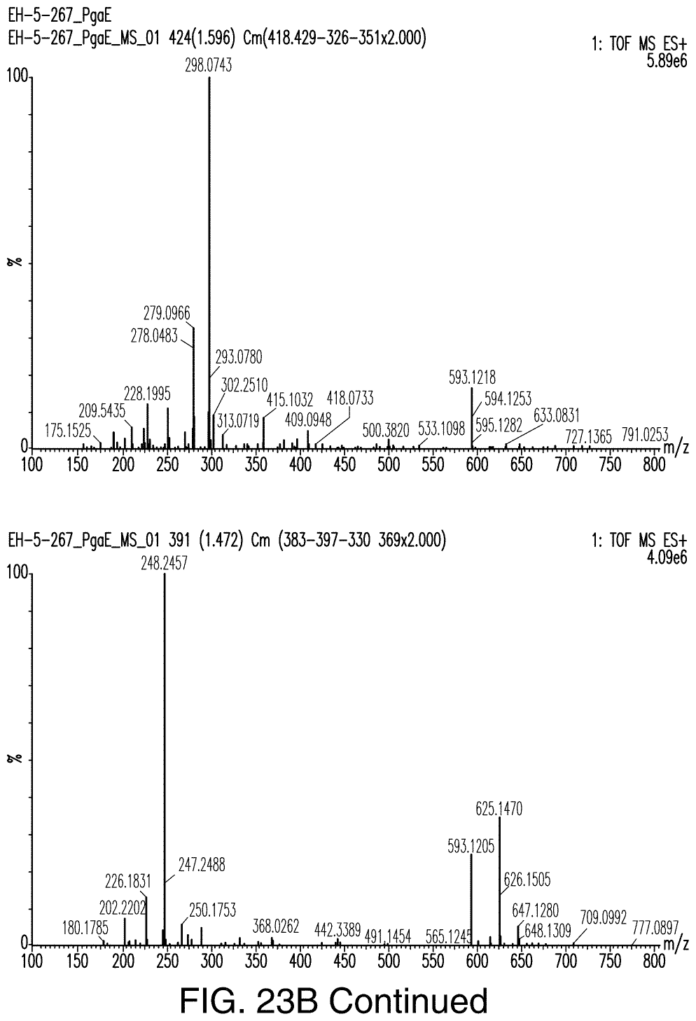

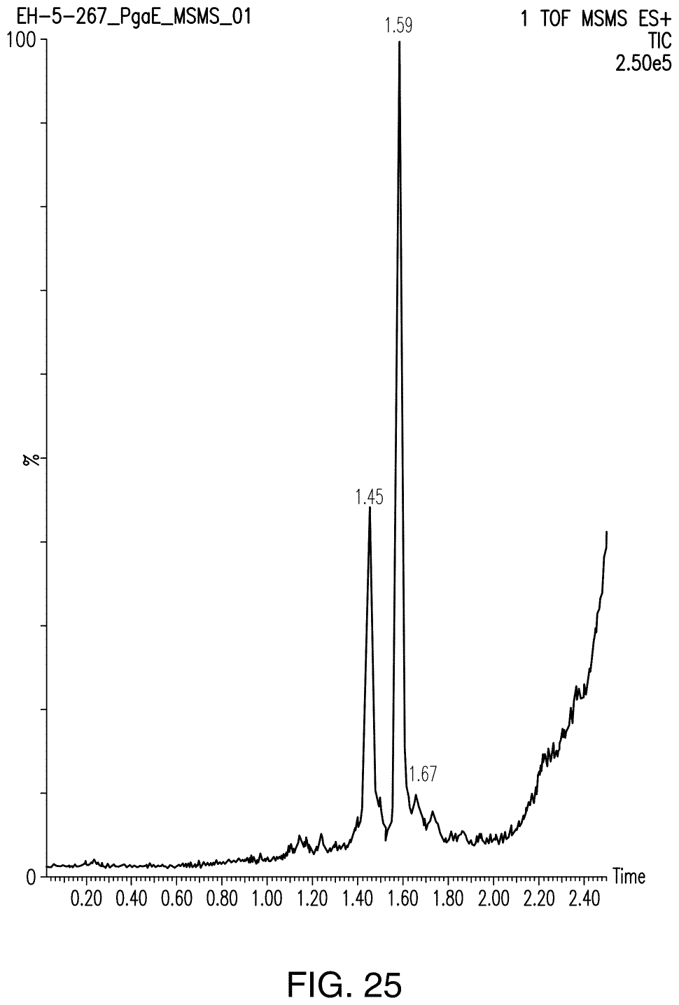

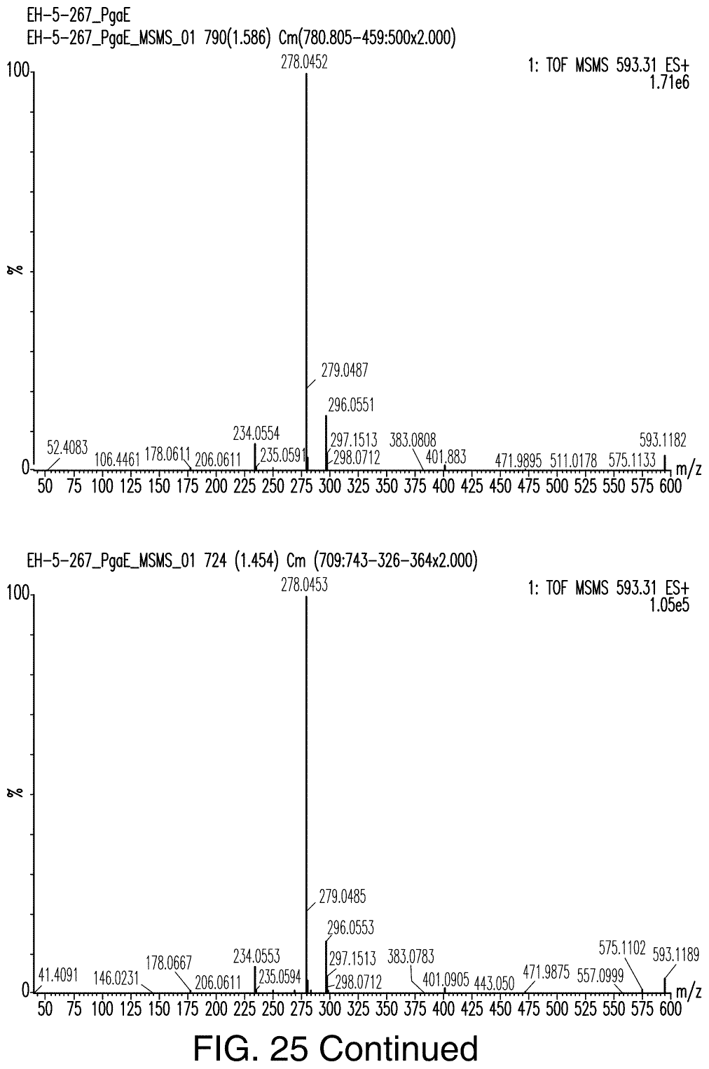

The present Example also provides for isolation and characterization of a new major product in a strain co-expressing PgaE and the TAN-1612 pathway that differs from the major product in the strain expressing the TAN-1612 without PgaE (FIG. 22, FIG. 23 and FIG. 25).

TABLE 9
Strains used in this Example.

| Strain | Genotype |
|---|---|
| BJ5464- | MATa ura3-52 his3-Δ200 leu2-Δ1 trp1 pep4 . . . HIS3 δ . . . |
| NpgA | pADH2-npgA-tADH2 prb1Δ1.6R can1 GAL |
| PBA-482 | BJ5464 ΔPEP4 . . . pTDH3-Sb-NpgA-tENO2 ΔPRB1 . . . |
| | pTDH3-Sb-NpgA-tENO2 |
| PBA-1277 | BJ5464 ΔPEP4 . . . pHSP26-Sb-NpgA-tENO2ΔPRB1 . . . |
| | pHSP26-Sb-NpgA-tENO2 |
| PBA-1333 | PBA482 pPBA-L2BWG9 |
| PBA-1337 | PBA1277 pPBA-L6BWG9 |
| EH-5-212-1 | PBA-1337 AL-1-101-C10 |
| EH-5-212-2 | PBA-1337 AL-234-C-C1 |
| EH-5-212-3 | PBA-1337 AL-239-B |
| EH-5-212-4 | PBA-1337 pSP-G1 (Addgene 64736) |
| EH-5-217-1 | PBA-1337 Plasmid Libraries A + C |
| EH-5-217-2 | PBA-1337 Plasmid Library D |
| EH-5-217-3 | PBA-1333 Plasmid Libraries B + C |
| EH-5-217-4 | PBA-1333 Plasmid Library D |
| EH-5-227-11 | BJ5464-NpgA AL-2-72-B-C6 |
| EH-5-227-12 | BJ5464-NpgA AL-2-72-D-C5 |
| EH-5-227-13 | BJ5464-NpgA AL-2-72-F-C4 |
| EH-5-227-14 | BJ5464-NpgA AL-2-79-J-C4 |
| EH-5-227-15 | BJ5464-NpgA AL-2-79-K-C5 |
| EH-5-227-16 | BJ5464-NpgA AL-2-79-L-C6 |
| EH-5-227-17 | BJ5464-NpgA AL-2-86-D-C5 |
| EH-5-227-18 | BJ5464-NpgA AL-2-86-I-C5 |
| EH-5-227-19 | BJ5464-NpgA AL-2-86-A-C1 |

TABLE 10
Plasmids used in this Example.

| | Description |
|---|---|
| pSP-G1 | pTEF1-FLAG-tADHI, pPGK1-MYC-tCYC1, 2 μ, Ura |
| AL-1-101-C10 | pTEF1-myS-tADH1 inpSP-G1 |
| AL-1-234-C-C1 | pTEF1-pgaE-tADH1 inpSP-G1 JCAT codon opt. |
| AL-1-239-B-C3 | pTEF1-ssfO1-tADH1 inpSP-G1 JCAT codon opt. |
| AL-2-72-B-C6 | pTEF1-Fungal-monooxygenase-6-tADH1 in pSP-G1^{c} |
| AL-2-72-D-C5 | pTEF1-Fungal-monooxygenase-5-tADH1 in pSP-G1^{c} |
| AL-2-72-F-C4 | pTEF1-Fungal-monooxygenase-1-tADH1 in pSP-G1^{c} |
| AL-2-79-J-C4 | pTEF1-Fungal-monooxygenase-4-tADH1 in pSP-G1^{c} |
| AL-2-79-K-C5 | pTEF1-Fungal-monooxygenase-2-tADH1 in pSP-G1^{c} |
| AL-2-79-L-C6 | pTEF1-Fungal-monooxygenase-2-tADH1 in pSP-G1^{c} |
| AL-2-86-D-C5 | pTEF1-Fungal-monooxygenase-8-tADH1 in pSP-G1^{c} |
| AL-2-86-I-C5 | pTEF1-Fungal-monooxygenase-11-tADH1 inpSP-G1^{c} |
| AL-2-86-A-C1 | pTEF1-Fungal-monooxygenase-12-tADH1 in pSP-Gl^{c} |
| EH-5-49-C | pTEF1-GAL1-dacO1-tADH1 inpSP-G1 |
| EH-5-80-6 | pTEF1-UBI4-IFNG-dacO 1 -tADHI in pSP-G1 |
| EH-5-80-7 | pTEF1-IFNG-dacO1-tADHI in pSP-G1 |
| EH-5-80-10 | pTEF1-oxyS-dacO1-31-tADH1 in pSP-G1 |
| EH-5-80-11 | pTEF1-oxyS-dacO1-87-tADH1 in pSP-G1 |
| pAV124 | Trpl, 2 μ acceptor vector for yeast golden gate |
| pPBA-AV124 | Modified Trpl, 2 μ acceptor vector for yeast golden gate |
| pPBA- | pHSP26-adaA-tPGK1 _pPGK1-adaB-tADH1 _pTEF1- |
| L2BWG9 | adaC_tENO1_pTDH3-adaD_-tRPL15A on pPBA- |
| | AV124 |
| pPBA- | pHSP26-adaA-tPGK1_pHSP26-adaB-tADH1_pTEF1- |
| L6BWG9 | adaC_-tENO1_pTDH3-adaD-tRPLl5A on pPBA-AV124 |

Plasmids Libraries. Plasmid libraries were used for making strains EH-5-217-1 through EH-5-217-4. The plasmid strains are shown in Tables 11-14. The yeast strains and the plasmids used are listed in Table 15.

TABLE 11
Plasmid Library A—Strain EH-5-217-1.

| Library | | Plasmid | |
|---|---|---|---|
| component | | common | |
| no. | Plasmid | name | Colony # |
| 1 | EH-5-49-C | Gal1-DacO1 | 1 |
| 2 | EH-5-80-6 | 9-ubilFN | H |
| 3 | EH-5-80-7 | 9-IFN | H |
| 4 | EH-5-80-10 | 11-OxyS-DacO1-37 | G |
| 5 | EH-5-80-11 | 11-OxyS-DacO1-87 | H |

TABLE 12
Plasmid Library B—Strain EH-5-217-3.

| Library | | Plasmid | |
|---|---|---|---|
| component | | common | |
| no. | Plasmid | name | Colony # |
| 1 | EH-5-49-C | Gal1-DacO1 | 1 |
| 2 | EH-5-80-6 | 9-ubilFN | H |
| 3 | EH-5-80-7 | 9-IFN | H |
| 4 | EH-5-80-10 | 11-OxyS-DacO1-37 | G |
| 5 | EH-5-80-11 | 11-OxyS-DacO1-87 | H |
| 6 | AL-234-C-C1 | PgaE in pSPG1 | 1 |
| 7 | AL-1-101-C10 | OxyS | |
| 8 | AL-239-B | SsfO1 | |

TABLE 13
Plasmid Library C—Strain EH-5-217-1 and EH-5-217-3.

| no. | Plasmid | Plasmid common name | Colony # |
|---|---|---|---|
| 1 | AL-2-72-B | Ate Moase Valsa Mali Var Pyr | C6 |
| 2 | AL-2-72-D | Ate Moase G Cichoaracearumi | C5 |
| 3 | AL-2-72-F | Ate Moase Valsa Mali CM | C4 |
| 4 | AL-2-79-J | Related to tetra hydroxylase | C4 |
| | | P subalpina | |
| 5 | AL-2-79-K | Ate Moase P subrubescens | C5 |
| 6 | AL-2-79-L* | Ate Moase P subrubescens | C6 |
| 7 | AL-2-86-D | Pentachlorophenol-4-Moase | C5 |
| | | Hypoxylon | |
| 8 | AL-2-86-I | Pentachlorophenol-4-Moase | C5 |
| | | Colletotrichum orbiculare | |
| 9 | AL-2-86-A | 2-ployprenyl-6-methoxyphenol- | Cl |
| | | hydroxylase Aspergillus | |
| | | Oryzae RIB40 | |

*AL-2-79-K and AL-2-86-L are the same plasmid but one of them does and the other does not encode a mutation in the sequence.

TABLE 14
Plasmid Library D—Strain EH-5-217-2 and EH-5-217-4

| no. | Plasmid | Plasmid common name |
|---|---|---|
| 1 | AL-2-57-A | F96X OxyS Library |
| 2 | AL-2-57-B | M176X OxyS Library |
| 3 | AL-2-57-C | W211X OxyS Library |
| 4 | AL-2-57-D | F212X OxyS Library |
| 5 | AL-2-57-E | T225X OxyS Library |
| 6 | AL-2-57-F | V240X OxyS Library |
| 7 | AL-2-57-G | P295X OxyS Library |
| 8 | AL-2-57-H | A296X OxyS Library |
| 9 | AL-2-83-A | A43X OxyS Library |
| 10 | AL-2-83-B | G297X OxyS Library |
| 11 | AL-2-83-C | G298X OxyS Library |

TABLE 15
Sequences used in this Example.
Sequences for the hydroxylases fungal hydroxylases of Table 17 are shown
capitalized within the context of the pSP-G1 backbone containing pTEF1, the FLAG tag
and tADHI (partial, uncapitalized).

| | |
|---|---|
| pTEF1- | tcaagtttcagtttcatttttcttgttctattacaactttttttacttcttgctcattagaaagaaagcatagcaatctaat |
| CM003104.1- | ctaagttttaattacaagcggccgcATGATGGGTGCAAAACACGTTATTAGAAGA |
| tADH1-in- | GTGACCAATTCGGAAAATGATACAGGATCAAGTTCGAAATCCACT |
| pSP-G1 | ACTTCATCTAATTCCGACAGGGACCATAATCCCTCTTCCTCTAATA |
| (Entry 1 | GAGACAGATATCACAATGAAACATCACCTGCCAATATGATATTGA |
| Table 17, | TAAACGAGCCACCTCCTACCACCTCCACAGAATTGGAGAGCGAC |
| AL-2-72-F- | ACTCATATTAACGTACGTAGCGTTTCTCCATGTTTGGAAACTGGTT |
| C4) | CCCTAAAAGCAGAGCAAGCGCCTCAGAATGTCCAGAACCCACCA |
| | CAGACACAGGACCAAGCTACTGAAGATACTGCCACCAACTCTCA |
| | ACCGGCCTCAACGCAGTTCAGAGCTATCATTGTGGGAGGTGGCCC |
| | AAACGGTTTGTGTCTGGCACATGCGTTATACCTAGCTGGTATTGA |
| | TTATGTTTTGCTAGAGCGCGGTGGTGAAATCATCAACCAATCTGG |
| | AGCCGGACTTGCTTTATGGCCTCATTCAGTTCGTATATTAGATCAA |
| | TTGGGTCTGCTAGACGAAGCTAGGAAACATTATTTCCCGATTAAG |
| | ACAAAGCATAACCACCGCCCCGACGGGTCAGTCAGAGATGTTAA |
| | CGATATGTTTGCCAAGGTAGAAGTTAATCACGGTCACCCCTGGAT |
| | GCTGTTCCATCGAGTAACATTGCTAGAACTCTTGTGGGAAAATTT |
| | GCCTGATAAAGAGACTAGAGTAAAAGTCAACAAGAAGGTGGAAA |
| | GTGTAGTCTCCAACCATGATGGCGTCGTGGTTACATGTTCAGATG |
| | GAACATCTGAAGCAGGAAGTATAGTGATAGGTTGCGATGGTGTA |
| | CACTCGACTGTTAGACAAGTCATGCATGACTTGAGAAGCGAGAA |
| | AAAGAAGAAAAGGCCAGGCCGAAAGCTCTCTCTGGGAAGGTCGC |
| | CGTCACAAGACAAGCCAATGAGAGCCCACTACTATGGGCTTATTG |
| | GTTGGATTCCGCTTCTTGATGGGTTACAACCTGCTGCTTGCTACGA |
| | AGTGAGAAGTGAGCCTAAAGGTAAAACCTTTACCATTTTAACGGG |
| | CGAAGATACCGCTTACTTCATCGTGTATATTCATTTAGAAAAACC |
| | CACGAGAGAGCGCAGCAGGCACACAGACGAGGATGCCGAAAGG |
| | TTGGCAGCCGCATTGGCCGAGAACAAGATAACGAGGGAAATTAC |
| | TTTTGGTGACCTATGGAGATCAAGGCGTTGGGGCAAAATGCTTGA |
| | TTTTCAAGAAGGGTTCGTAGATAAATGGTATCATGAAAGAATCGT |
| | TTTAGTTGGCGATGCTGTCCATAAGATGACTCCAAATGCGGGACT |
| | AGGCTTGAATGCTGGCTGGCAAGGTATCGCTGAATTAACGAACA |
| | GATTGCGGAGATTAGTTGTTGCAGAAGGACAAAGACCAGATGCA |
| | AGGTGCGTTGAAAAAGTTTTTCGAGGTTACCAAGATAGTAGAAA |
| | GGGTATGGCAAAGAAAACTATGAGGTTTTCTTCTCTGTACACCCG |
| | TGTGGTAGCTAATCAGAGCTTACTGTATCGTTTTTGTGATAGAAT |
| | GACACCAGCGGTTGGTGGGGATGTCGCATTATTAAATACAATGGC |
| | TAGTCCTATTGTTAAAAAAGGTGTCACTTTCGACTTTGTAGCGGA |
| | ACGTGATCATAAAGAAGGTAGAGTAAGATGGGTTCATCCACAAC |
| | ATGTTCCAGCTGAAgtatcgatggattacaaggatgacgacgataagatctgagctcttaattaac |
| | aattcttcgccagaggtttggtcaagtctccaatcaaggttgtcggct (SEQ ID NO: 64). The |
| | capitalized sequence is SEQ ID NO: 77. |
| pTEF1- | tcaagtttcagtttcatttttcttgttctattacaactttttttacttcttgctcattagaaagaaagcatagcaatctaa |
| MNBE010 | tctaagttttaattacaagcggccgcATGACCAAGTCCAAGTTATACGACGTGAT |
| 00128.1- | AATCGCGGGCGCCGGACCCGTCGGCCTCTTCCTCGCCCACGAAC |
| tADH1-m- | TCAGTCTCCGCCAAATAGCAGTCCTAGTCCTAGAACGAACAACC |
| pSP-G1 | CAACAAGACTCGCCCTGGAAATCCGGACCTCTAGGCCTCCGAGG |
| (Entry 2 | CCTAAACATCCAATCCATCGAAGCATTCTATCGCCGCGGTTTACT |
| Table 17, | AGGCCAATTATTCAATCTGGACGAAAGGCAAAAGTACCAACCGA |
| AL-2-79K- | GCAAGAAACCAGGTTTCCAATTCGGCGGCCACTTCGCCGGCATC |
| C5 or AL- | ATAATAAATGCCAATCAATTCGATTTGCAACGCTGGAAGTACCG |
| 2-79L-C6) | CCTCTCAGGTCCGGCGCTTGGTCCGGGTCCAACGACGATGGATC |
| | AAGTGGAGAAGGTCCTAACGGCGCGGGTGGAGAGTCTAGGCGT |
| | GAAGATTCTCCGCGGTCGATCTGTTAGCCGTATCTCGCAGGATG |
| | AGGCAGGCGTTACAGTCGAAACAGACGATGACCATTCCGAGTCA |
| | TTTCGGGGCAAATGGCTCGTTGGCTGTGACGGTGGGCGCAGCGT |
| | TGTTCGAAAAGAAGCCGGCTTCGAATTCGTCGGTACAGATGCCC |
| | AATTCACCGGCTACGCAACGCATGTCGAGATAGAAGGTCGCGAG |
| | AAGTTGAAGCCTGGTCTCAACGTTTCTGAAACGGGGATGTATAT |
| | CAACGTAGTGCAGAATGGCGCTTTTCATCTCCTTGAATTTGACGG |
| | GGCGAACTTCGATCGCGCGGGTGATATCACGATCGATCACCTTC |
| | AGCGGGTCCTTGATCGGGCTATTGGTCGGACAGATGTGAAGATT |
| | ACAAATGTGCATCTTGCTTCTTGTTATACCGATCGTTCAATGCAG |
| | GCGGCAAGTTACCGGAGGAACCGCGTTCTTCTTGCCGGTGATGC |
| | AGCGCATATTCATTCTCCGCTCGGCGGGCAAGGACTGAATGTCG |
| | GACTAGGTGATGCGATGAATTTGGGGTGGAAGCTTGCGGCTATA |
| | GTTCAGCGGGACCAAGGCCAGGGAGAGGATTCTGGGAATACGC |
| | ATGATCTGACTCTTCTCGATACCTACGAGAGCGAGAGACACCCC |
| | ATTGCTGCGTCGGTTCTTGATTGGACTCGTGCGCAGGTCACGGCG |
| | CTGAGGCCGGATGCCTTTGGTCGGGCTACGCGCGCCCTGACGGA |
| | AGATCTGATCAAGACGGATGATGGGGCTAATCTGTTTATTGATC |
| | GGATCTGGGGACTTTCGCAGCGATATAAACTTGGCGAAGAGTCG |
| | AGGCCTACACATCCGCTTCTTGGGTCTAGTGCGCCTGATTTCGAG |
| | TTTGGGAATGGGTCTAGGCTTGGCTCGAAGCTTGAGGAGGGACG |
| | GGGGTTACTTCTTGATTTTGGGAACAGTAGTGAACTGGAATCAG |
| | TTGTTGATAGAAAATATTCTGGGAAGGTAGATTATCTTGCTCCAG |
| | AGGCAAAGGAGAACTGTGGGCTGAGTGCCTTGTTGATTCGACCT |
| | GATGGAATCGTTGCATGGGTGGTGGAAGAAGATGCGCAGCACG |
| | ATATTGATGCTTTGAAAGCTGCATTAGGGGATTGGTTCACTTTGgt |
| | atcgatggattacaaggatgacgacgataagatctgagctcttaattaacaattcttcgccagaggtttggtcaa |
| | gtctccaatcaaggttgtcggct (SEQ ID NO: 65). The capitalized sequence is SEQ |
| | ID NO: 78. |
| pTEF1- | tcaagtttcagtttcatttttcttgttctattacaactttttttacttcttgctcattagaaagaaagcatagcaatctaa |
| NC_ | tctaagttttaattacaagcggccgcATGACAACTAATCCAGAAGTCATCATCGT |
| 036772.1- | GGGCGGCGGAGCAGTCGGCCTGGCCGCTGCATGCGAATTGGCCA |
| tADH1- | TCAAGAATGTCTCTGTTACGATCTTGGAGCGGGAATCTGGACCT |
| in-pSP-G1 | GCAGCGCCATGGAAGAACGAGAAGCTGGGCTTTCGCGAGCTGTT |
| (Entry 3 | TGTGCCTGCCATCGAGCACTTGTACCGCCGTGGCCTGCTGAACG |
| Table 17) | ACATGTTCCGCAATGAAGAACGACCACAGACTATGCCGGAGAAG |
| | AAGGGTGGCTTTGAGTTTGCTGGTCATTTTGCTGGTATGATGATC |
| | AATGGAAACGATGTCAACTATTCACACTGGGACGATACACACCT |
| | TCCCGGGCCTGCTTGTGTACCTGGATCTTCGACACTGGGTCACAT |
| | TGAGAATGTTCTGCGTGCACGTGCTGAGCAACTCAAAGTGCCTA |
| | TTCTTACTGGCAAGAATGTGACTGGCATCGAAGACACAGACAGC |
| | GGCGTCAAGGTGTTCTGCGGCGAGGAAACATTCGAGGCTCAATA |
| | TCTTGTCGGATGCGATGGTGGTCGGAGCACCATTCGCAAGAAAG |
| | CTGGGATTCCATTCATCGGCACAGAGCCCGAGTTCACTGGCTAT |
| | GCTGTCCATTGCAAGCTCGACAACCCCTTTTTGCTCAAGCCCGGC |
| | TTCAATCGATGCCCAAATGGCGGACTCTACATCATGGCCGGCCC |
| | ACAGCACATCTATGCTGTAGATCACGACATCAGCTTTGACCGAG |
| | ACCAAGAAGTAACAGCGGAACACTTTCAAAAGGTTCTGAGAAA |
| | GCTTTCCGGTACCAACGTGGTTGTTGAGCAGGTCGTTCTCGCATC |
| | CGCTTTCACCGATCGCTGCAGACAAGCTGAGCAGTACCGCAAAG |
| | GCCGAGTCTTCCTTGCTGGCGATGCTGCCCACATCTACCCTCCAT |
| | TCAGCGCCCAAGGACTCAACTGCGGTTACATCGATGCAGTGAAT |
| | CTCGGTTGGAAGCTCGCTGCTTTGGTCAAGGGCTATGCTACTCCG |
| | GGCTTGATCGATACATACCAGCAAGAGCAACACCCAATGGCTGC |
| | GCGAGTACTTGATTGGGTGCGAGCACAAATTGTTACCCTTCGAC |
| | CTGACGCTCATGGCCGGGCTGTGGGCAATGTTATGCGCGAGTTC |
| | CTCTACACCGAAGCTGGTGTGACATACTGCATCGGTCGACAATG |
| | GGGTCTTGATGTGCGGTATGAGATCGGAGAGGGACATAAGCTTG |
| | TCGGTCGCAGCGCTCTGGATTACGAGCTGGACGATGGCTCTCGA |
| | CTGGGAACTAAGCTCGGCGCCGCGAACTTCACACTCGTGGCATT |
| | TGGTGACTGCAGTTCAGAGCTCACCAGCTCCGTCAACGAGCTTA |
| | ACCCAGTGCTCGGATTCTGCAGAGCGAAGGGCGAGGAGAAGTC |
| | AGGCCTCCAGGGCGTCCTTGTACGTCCTGACGGGATCGTGTCGT |
| | GGGCATCTGCAGATGCCCTCAACATCGAGACGATCAAGGCCAGC |
| | CTCGAACGCTGGGTCGCGCTGCCAGCTGCTGTTGAGAAATTTGA |
| | ATCTGTGTCTTACGAGGCCAACCAGTTGTCCAACATTCAGACGCC |
| | ATTGACGACTACTGTTCGATCGAATATCGGACGTGTATTTGGCAA |
| | GGATGGAGAGAAGTACACTGGCCTTCCGACAGTGGTGACGGAA |
| | GTCCCGGCAACGGCTGATGCAgtatcgatggattacaaggatgacgacgataagatctg |
| | agctcttaattaacaattcttcgccagaggtttggtcaagtctccaatcaaggttgtcggct (SEQ ID NO: |
| | 66). The capitalized sequence is SEQ ID NO: 79. |
| pTEF1- | tcaagtttcagtttcatttttcttgttctattacaactttttttacttcttgctcattagaaagaaagcatagcaatctaa |
| FJOGO10000 | tctaagttttaattacaagcggccgcATGGGCGAGACCGATGTTCTGATAGTTGG |
| 08.1-tADH1- | TGGAGGGCCCACTGGATTGATGCTCGCCCTCGAGCTTACCAGCC |
| in-pSP- | AAGACATCCCATTCCGCATCATCGACTCAAGTCCTATTCGAAGC |
| G1 (Entry 4 | GACAAAAGCCGGGCCTTGGTTCTCCATTCACGGACTCTGGAACT |
| Table 17, | TCTCAATCGCCATGGAATTGCTCAGGAGTTCGTTGACCGTGGAAA |
| AL-2-79-J- | TTTCAATTTGGCCGTGCGCATCTTCGCCAACCAGAAGTTCGTGTTT |
| C4) | GAGAACGATTTCAACATCATCGCTTTCAATGACACTATGTACCAG |
| | AACCCCTTGATTATCAGTCAGGCAGAGATTGAATCGATCCTGGAC |
| | GAAGTCTTGATGAAACATCAAGTGAAGGTGGAACGGCCCGTGAC |
| | AGCGGAGAAGATATCACAAGATGAGACTGGAGTTACTGCCGTAC |
| | TACGCCACGAGGATGGAAGCGAAGAGTCACTCCGTTGCAAATAT |
| | GCTGTAGGCTGTGACGGCCCTCGCAGCATTGTACGAAATTCGGCA |
| | GGTCTACAATTCGAAGGAGCGGCGTACCCGCAGGATTTCATCCTC |
| | GCAGATGTGCACATGAAATGGGAGACTAGAGAATGCCTTCATAT |
| | CTATCTTGGCGCCTCTGGGTTCATGATTGTCTTTCCGATGAAGGAC |
| | AATGTCTGGCGTCTGATCTGTTCTCGCAGGGAAGCACTGGGCGCT |
| | GACGCTGAGCCAACTCTTGAGAATTTCCAGGAGATGTTGGACAAG |
| | CTGCTTCCTGAACCTGTTCAAATATTTGATCCAGTTTGGATCAGTC |
| | GATTTAAATTACACCATCGAATTGCAGACAATTACCGCGCTGGTC |
| | GAATGCTCGTCGCGGGCGATGCAGCCCATGTCCATTCTCCTGCTG |
| | GTGGGCAAGGGATGAACACCGGTATGCACGATGCAGTCAATCTT |
| | GGCTGGAAGCTTGCAAGTGTCATTCGGGGCGAGAACGATGACTCT |
| | CTGTTGGACTCGTACAATATCGAACGGCGTAGAGTTGGCCAGACT |
| | CTCCTGCAAGGCACAGATAGACTTTTCGAGTTTATGGCAACAACA |
| | AATCCTTTGTATCTCTTCATAAGGAACTACGTCATGCCTTTCATTA |
| | TGCCCTGGGCCATGGCGATACCTGGCCGTCGAGCTCTTGCATATA |
| | GATTTGTATCCGAGTTGGGGATTCGATATCGCAAAAGTCCCATCG |
| | TTGGACAGGCAACGACTTGGAAAGGAACACTGAAAGGTGGAGAT |
| | CGAGTACCCGACGGGAGACTCCTGAAGGGGAATATTGAGATCAC |
| | TGTTCACTCGTTGCTTGGAGCACGCAAGCACAGTCTACTTCTGTTC |
| | TCGGGCGTGGACGGGGTCACCAGCACAGACGATCTGGAGAAAGC |
| | TCATGTGGAGTTCATCGAGGCCAGTGGTCGGTCGATCCCAGTATA |
| | CACAATCACGAAATCATCCTCGGAGAACATAGAGATCATGGATC |
| | CGGAGGGGCTAGTGCATAGGCTGTTTGGCTTTACAGCATCCGGCT |
| | TTGTGTTAGTTCGACCCGATGGTCACATTGCGTTCATCGGGCCTCT |
| | CACGTCAATGGACGAGCTCAAGACGTGGATGGAGAGAgtatcgatggatt |
| | acaaggatgacgacgataagatctgagctcttaattaacaattcttcgccagaggtttggtcaagtctccaatca |
| | aggttgtcggct (SEQ ID NO: 68). The capitalized sequence is SEQ ID NO: |
| | 80. |
| pTEF1- | tcaagtttcagtttcatttttcttgttctattacaactttttttacttcttgctcattagaaagaaagcatagcaatctaat |
| MCBR010 | ctaagttttaattacaagcggccgcATGGATTGTGAAGTATTAATTATCGGTGCT |
| 07648.1- | GGGCCCACAGGACTTATGCTTGCCCTCGAACTTTCCCGCGAACAA |
| tADH1-m- | ATCCCCTTCCGAATTCTCGATAGTCATTATTCTACTAAGACAGCC |
| pSP-G1 | CATCAATCTCGGTCTATAGTGGTTCACTCACGTTCCCTGGAGTTA |
| (Entry 5 | CTTGCCCGGCATGACTTAGCCGAAAGCTTCATCGCTAAAGGATTA |
| Table 17, | CTCATGGAAGAGATTCGCTTCTTCTTGAAGCAAACCCTTAATGGG |
| AL-2-72-D- | AAGAGTAGTTACCCTGGAAGTCTCATGAATGATACAATTTATAAA |
| C5) | AAACCACTTATCATTAGACAACCACTATCAGAGCAGCTTCTGGAA |
| | AAACGTCTTTGCGAGTATGGAGGCATTATTGAAAGGGGGGCAAG |
| | CGCTAAAAAAATGGTGATAGATGAAGATGGCGAGCGGGTGTCTG |
| | TTTTGGTACAGCATAGAATTCAGGAAGAAGAAGAGAAAGTAAGA |
| | GAGGAATTAATCAATTGTAAGTATGTTGTAGGTTGTGATGGAGCT |
| | AACAGTATGGTACGAAAATTTGCGGGTATCGACTTTCCGGGTACT |
| | TTATACCCTCAAGAGCTCATTCTCATTGATGCACAGGTAGACTGG |
| | AACAATAAAATATGTCCACATGTTTTTATTGATAAGGAATTCGTG |
| | ATCTTTATACCGCTAGACGAAACAGGTATCAACCGAATCATCTGC |
| | CGGCGTCCTCCAAATGACACCAGCACTATCTCATCCAGATCAAAT |
| | CTGTTATCAGGATCATCGCCTTACCAAGAGCCTAGCCTATCCGAG |
| | TTTCATGATACAGTTATTTCTTCCGTTCCTGGTACGACTCGTATTC |
| | ACAGCCCGATTTGGTCTTCCTCATTCCGTGTCTCACGTCGACTTGC |
| | TAGTACATATAGTGTGGGGCGGATCTTCCTTGCTGGTGACGCCGC |
| | TCATGTCCATGCACCTATTGGCGGTCAAGGTATGAATACCGGTAT |
| | TCAAGATGCCGTAAACCTGGGTTGGAAACTTGCGCGTGTTATAAA |
| | ATCAACTGCTCCGTCCTCTTTACTCAACTCATATAATGTAGAGCG |
| | ATATAAAGTTGGTAGTGACACTGTTGAAAATACAGATCGCATGTT |
| | CAATATTTTAATAACTACAAACCCTGTAAAAATCTGGTTACGAAA |
| | CTTTCTTTTTCGCTGGGTTTTGCCATATTTCCTCAAACCAGATTTTG |
| | CAGACAGAAAAATTCGTTACATATCACAATTGTCAATTCGATATC |
| | GAAATAGTCCCGTTGTAGGCACGGCTTCTGTGTGGAAAGGAATTC |
| | TAAGAGGCGGAGATCGGGCACCAGATGGATGGATGATGAATCCT |
| | GAAGGAGAAAATATCACTTTGCACAGGCTTTTTAGGACATCAAAT |
| | GATCATCTTTTCTTATTCAGCGGCCTAGACACTCTTGAATCGCGGA |
| | TGGTCAATGATACTGTCCAGCAGCTAAAACTCTGCAGCTCAGTTC |
| | TTGTGGTACACAAAATATATGACGGGAATTTTAAAGATAAAACA |
| | ATTATCGATGGTTACATAGACCCAGGGGGTAAAGTTCACACCTTG |
| | TATCAGTTTATTGAGCCTGGTTACGTTCTTGTTCGACCCGACGGAT |
| | ATATCTCATTTATAGGACCTATGACTTCACTAGATGAACTGAAGA |
| | ATTGGATGAATTCATATATGCAGGGAgtatcgatggattacaaggatgacgacgata |
| | agatctgagctcttaattaacaattcttcgccagaggtttggtcaagtctccaatcaaggttgtcggct (SEQ |
| | ID NO: 69). The capitalized sequence is SEQ ID NO: 81. |
| pTEF1- | tcaagtttcagtttcatttttcttgttctattacaactttttttacttcttgctcattagaaagaaagcatagcaatctaa |
| KN714671.1- | tctaagttttaattacaagcggccgcATGGAGGCTAAGAAATCTTTCAAAGTTAT |
| tADH1- | CATTGCTGGTGGCAGTATCGTCGGCCTAACGCTGGCCAACGCGC |
| in-pSP-G1 | TCGAGAAGGCGGGTATTGATTTTCTTGTCCTCGAGAGGGGCGAC |
| (Entry 6 | ATTGCTCCACAGCTCGGCGCATCAGTCTCAATCTACTGCAATGCG |
| Table 17, | TCCAGAGTCCTCGACCAGCTGGGAGTTTGGAAAGGCATTCACCA |
| AL-2-72-B- | GAGCACAATCCCGCTGACTGATCGGCTATACTTCGATGAACATG |
| C6) | GCCGCTTGTTTGAGGACAACAAGATGATGAGGCTCATAGACGAC |
| | AAGACTAAGCGGCCTATCACGTTCTTGAGAAGGGAGGCATATAT |
| | CAGGGCTCTTTACGAAAACCTGAAGGACAAGTCGAAGGTGAAG |
| | GGTCACTCTGGGCTGGTGTCCTTTGCCGAAGATGATGAAGGCGT |
| | TTCAGTATTCACCAGTACCGGCGAGGAAATCAGGGGCAGCATTC |
| | TTGTGGGAGCAGACGGCATCCATAGTACCGTCCGGAAGCTCATG |
| | GCTGAGTCTGTCGCCAAGTCGGACCCTCAGAGGGCTAAGAATCT |
| | CATCGAGGGCTTCACGGCAAACTACCGGACGATCTTCGGAACGT |
| | CCAAAAACCAGCGAAAAGACGACCCGAGCGTGCAGATGGTGGC |
| | AGATAGTCTTTCCCATACCTCCTATTACCGTGGCGTCACGGGGGT |
| | CTGCGCCGTCGGCAACAGTGGGGTGATCTACTGGTTCCTCTTGGT |
| | GAAGGAGGACACGCCTTCGACAATGCCGAACTGCCCGCGCTACT |
| | CGGAGGCGGATGCCGAAGAGACGCTTCGTAAATTCGGCCACCTT |
| | CATATGGGACCGGGATATACTTTCAATGACCTCTGGGAACTGAG |
| | GGAGAAAGGAGTCATGGTACCGTTGCAGGAAGGCATCGTCGAA |
| | GGAAGCTGGAATAGTAGCGGGAGGGTTGTACTCATGGGCGACGC |
| | CGTGCACAAGTTCACTATAAGCGCGGGGCTGGGCGCCAATTTAG |
| | GCGTAGAAGGCGCTTGCCACCTGGTGAATGAGCTCCTCCCTGTCC |
| | TCAAAGAGACGGAAAACCCCGGAAAGCAAGAGATAAAGGATGT |
| | GCTCGACAGATACGAGGAGAAGCATCGTCCTCGAACTAAGATCT |
| | GTGCCGTATTGTCTAACTATTTGACCAAGTACGAGGCGATGGAGA |
| | CCTGGTGGCTCAGACTTCTGAGGTTCATTATTCCTCGGATCCCGG |
| | ACAGTTACAAGGCGAAGTCATTTGTGGACTTCATGTATGGTGCTC |
| | CGATTCTCGATTTCCTGCCTCACCCGGGCAAGAGCTCTGCGgtatcgat |
| | ggattacaaggatgacgacgataagatctgagctcttaattaacaattcttcgccagaggtttggtcaagtctcc |
| | aatcaaggttgtcggct (SEQ ID NO: 70). The capitalized sequence is SEQ ID |
| | NO: 82. |
| pTEF1- | tcaagtttcagtttcatttttcttgttctattacaactttttttacttcttgctcattagaaagaaagcatagcaatctaa |
| KL584983.1- | tctaagttttaattacaagcggccgcATGTCGGTCTTACAATTCAATCTGAACGG |
| tADH1-in- | CTTCGTGCCTGGCGACCCTCATGATCTCTACCGTGTCCACCAAGA |
| pSP-G1 | AACCGCCGAGATCGCAAACAATGTCCCGCAAGAAGTCGACGTCC |
| (Entry 7 | TCATCATTGGCTCTGGTCCAGCTGGTCTGGCTCTAGCAGCACAGC |
| Table 17) | TATCTCTCTTTCCAAGCATCACCACAAGAATCATTGAACAAAAG |
| | GCTGAACGACTAATCCTAGGCCAAGCCGATGGCCTCCAAGTCCG |
| | AAGCATCGAAATGTTCGAAGCATATGGCTTCAGCGAAAGAGTCC |
| | TCAAGGAAGCGTACTACATCAACGAAACCACCTTCTGGAAGCCT |
| | GACGAGCAGCAACCGGACCGCATCGTGAGAAGTGGCAGGATTA |
| | AGGATACTGTAGACGATCTCTCCGAGTTCCCGCACGTCATCCTCA |
| | ATCAGGCGAGAGTTCATGACTTCTTCCTTGATGTCATGCTCATGT |
| | CGCCGACAAGATTGCAACCGAGCTATTCACGCAAACTCAAGCAG |
| | CTCAGCCTTCAACCAGCACAGGGCAACTCCTCGCACGCTACGTC |
| | CTCCGTGCAAGTTCAACTCGAGAGAACAGACTCCAGCCACGAGG |
| | GCGAAGTCGAGGATGTACGTGCTCGCTACGTCGTAGGCTGTGAT |
| | GGAGCCCGCAGTGCCGTCCGTCAAGCCCTCGGTCTCAAGCTGGA |
| | AGGCGACTCCGCAAACCAAGCTTGGGGTGTCATGGACGTACTTG |
| | CTGTAACCGACTTCCCAGACTGGAGGACCAAGGTTGCAATCCAC |
| | TCCGCGACACAAGGCAGCATCTTGATCATCCCCAGAGAAGGAGG |
| | TTACCTTGTGCGCCTCTACATTGAACTCGACAAGCTCGACGCCAA |
| | CGAACGTATCGCAAACAAGAAGATTACCGCCGACAAGCTCATCG |
| | AGACCGCACAGCGCATCTTCCATCCTTATGCTTTGGATGTCAAAG |
| | AAGTAGTCTGGCACTCTGTCTACGAGATTGGCCAGCGCCTGTGT |
| | ACCAGATTCGACAACCTCACTCCGGAGACCGCTACAACAGAATC |
| | CCCAAACATCTTTATCGCAGGCGACGCATGCCACACCCACAGCC |
| | CTAAAGCAGGTCAAGGCATGAATGTCAGCATGCAAGATGGCTAC |
| | AATCTAGGCTGGAAGCTCGCCTCCGTGCTCAATGGAATCTCTAA |
| | CCCGGCCATCCTCCACACCTACTCCGCCGAACGTCAAAAAGTCG |
| | CTCAAGACCTCATCAACTTTGACCGAGAGATTGCTTCAATGTTTA |
| | GCGCCAGACCCAAGAGCCATGCCAAAGACACAGAGGGAGTCGA |
| | CCCTGCTGAATTCCAAAAGTACTTTGTCAAGCAGGGCCTCTTCAT |
| | GGCTGGACTGGGCACAGCTTACTCGACTTCTGCTATCACCGCTGG |
| | ATCAGAGCATCAACACCTGGCCAAAGGTTTCCCCATCGGCATGA |
| | GATTGCACTCTTCTCCAGTCATAAGATTGGCAGATGCAAAGCCG |
| | ATCCAACTAGGTCATGTGGTAAAAGCAGATGGAAGATGGAGAGT |
| | CTTCGTCTTCGCATCAGCCGAGGATCCCGCTTCGACCTCGTCAAG |
| | CTTCCACTTGGCCTGCGAATCCCTGACCGAGCTTGTCAACAAGGT |
| | CAACCCTACTGGTGCTGATATCGACTCCGTCATCGATGTCAGAG |
| | GTATTCTGCAGCAGGGCCATGCTTCACTGAACATCAACCACATGC |
| | CTGCTATCGTGTGGCCGCAAAAGGGAAAGTATGGACTCAGAGAT |
| | TACGAAAAGGTCTTCTGTGCTGAAGATGTGGATGGACATAGGGA |
| | TATCTTTAATGCTAGGGATATCGATCGCAGAGGCTGTATCGTGGT |
| | CGTACGACCTGACCAGTACGTCGCCAACGTTCTGCCGTTGTGCGA |
| | TCATGATGGTTTGTCAAGGTTCTTCGGAGGTTTCATGTTTGCAAG |
| | GGATgtatcgatggattacaaggatgacgacgataagatctgagctcttaattaacaattcttcgccagag |
| | gtttggtcaagtctccaatcaaggttgtcggct (SEQ ID NO: 71). The capitalized |
| | sequence is SEQ ID NO: 83. |
| pTEF1- | tcaagtttcagtttcatttttcttgttctattacaactttttttacttcttgctcattagaaagaaagcatagcaatctaa |
| KZ111246.1- | tctaagttttaattacaagcggccgcATGACCAAGACTTACGATGTTATCGTTGC |
| tADH1-in- | CGGCGCAGGCCCCATCGGCTTGTTTCTCGCCTGTGAGCTGAAGTT |
| pSP-G1 | GGCGAACGTTGACGTGCTAGTACTAGAGCGTGACACCAACCCCG |
| (Entry 8 | AATCCCCCTGGAAAAGCGAACCACTCGGCATACGAGGCTTGAAT |
| Table 17, | ACGGTATCTGTCGAGTCCTTCTATCGACGTGGGATGCTAGACAA |
| AL-2-86-D- | GGTCACCTCCATGGAGCGACCGAACTTCTTCAAGAAAGGCCCGG |
| C5) | GGTTCCAATTCGGAGGCCACTTCGCCGGCATGCTACTGAATGCC |
| | AACAAGCTCGAAATGTCCCGTTATAAGTACCGCCTCCAAGGCCC |
| | AGCATTGGTTCCCAACGGATCAAGTATGGAACGTGTTGAGAAGG |
| | CCCTAACAGAGCGCGCTGAGAGCATTGGAGTAGATATTCTCAGG |
| | GGGAAGGGAGTCACGGAAGTCTCGCAGGATGACAACGGCGTTA |
| | CGGTGGAAGCAGGAGGCGAGACTTTCCATGCGAAATGGCTTGTC |
| | GGATGTGATGGCGGGCGGAGCACAGTTCGCAAGGCCGCTGGCTT |
| | CGACTTCGTCGGAACAAATCCTGAATGTACCGGTTACGCTTTTTT |
| | GGCCGATTTAGACCATCCCGAAAAGCTGAAGCCGGGGTTCCAAC |
| | CCTCTGTAGGCGGCATGTACATCATAGGACATTGGGGTAACATG |
| | TACTTGCTAGACTTCGATGGTGGCGCTTTCGATCGTACGCAGGAG |
| | ATCACACCCGAGCACCTTAACGAAGTCATGGCCCGTGTGACAGG |
| | CACCGACGTCAAGGTCACCAAGATTCATCAGGCATCCTCGTTTA |
| | CCGACCGATCTAAACAAGCGTCGACTTATCGAAAAGGCCGCGTT |
| | CTCTTAGCCGGCGACGCGGCGCACATCCATTCGCCTCTGGGAGC |
| | ACAAGGATTGAATCTAGGGTTGGGAGATGCCGTGAACCTTGGCT |
| | GGAAATTGGCAGCGACAGTTCAGGCTTCTAAATCTGGAGAGGAA |
| | CCAAAGGACCTCAGCCTACTCGATTCCTACGAGAAAGAGCGATA |
| | TCCGATCGGGTCTTGGGTTCTTGAATGGACACGCGCTCAGATCTC |
| | GACGCTAAAGCCCGACATGTTCGGTATCGCTCTTCGTAGGCTCAT |
| | GAGCGATTTATTGGAGACGACTGATGGAACGAATTTATTCATCG |
| | ATCGCATCTGGGGTCTGTCGCTGCGGTACGATCTTGGCGACGAA |
| | CACCCCGCCGTCGGCTGTAGCGCTCCCGATTTCGAATTCCACGAC |
| | GGAACGAGGCTAGGTCCCAAGCTAGCGACGGGGCGAGGTCTATT |
| | GCTCGATTTCGGATCTAATGCAGAACTCAAGGATCTTGTCGGGA |
| | AATACGAGAGCAAGGTTGATTATCTTAGCACAGAGGCAAAGGAC |
| | CAGCTGGGCCTGAAATCCTTGTTGATCCGCCCCGATGGCGTTGTG |
| | GCTTGGGTGGCTGAAGAGAAGCCTGATATGGACTCGGCGAAGGC |
| | CGCTCTGGAGAGATGGTTTGGGCTGGGTTCGAAAgtatcgatggattacaa |
| | ggatgacgacgataagatctgagctcttaattaacaattcttcgccagaggtttggtcaagtctccaatcaagg |
| | ttgtcggct (SEQ ID NO: 72). The capitalized sequence is SEQ ID NO: 84. |
| pTEF1- | caagtttcagtttcatttttcttgttctattacaactttttttacttcttgctcattagaaagaaagcatagcaatctaat |
| FJOGO10000 | ctaagttttaattacaagcggccgcATGGAAAACCAGCTCTACGACGTCATCAT |
| 31.1 tADH1- | TGTTGGCCGCGGTCCTGTTGGGCTATTTTTAGCATGCGAACTTCG |
| in-pSP-G1 | CCTCACAGGGCTCTCTGTCTTGGTTGTTGAGCGCCGCTCGAGCTC |
| (Entry 9 | AGAGGGAGTGGCGGAGACTCGCGCTTTTGTTGTCCATTGCCGCAC |
| Table 17) | ACTCGAGATCCTCGCATTCCGTGGTCTTCTTGGCAAGTTCCTCCAG |
| | GAGGGCACGAAGTCTCCTTGGTGGCACTATGGCGTCTTGGACACC |
| | CGTTTGGATTTTAGCAGATTTGGCCACGAAACGAAGCAGAATTTT |
| | GTCCTGTTAAGCCCACAGTTCAGAACTGAGGAGATTTTTCTGGAG |
| | CGTGCGAAGGAATTAGGCGTCGACATTGTTCTGGGAATTCGCGTC |
| | GAGTCCGTGGAGCCTTCGACTACGTCTGTTACGATATATGGAACA |
| | TCAAGCAGTCCAGATGGCGGCGCTAGAAGTTCGTTCAAGGCGAG |
| | AGGAAAGTATCTTGTCGGTGCGGATGGAGTGCGCAGTACCATCCG |
| | GAAGGCTACGGGCTTTGAATTCCCAGGTACGCCGGCAACACACA |
| | CGGGTTTGACCGGCGAGGCAACATTGGGAGTGCCCATGCCCTATC |
| | CCTACATTGCCAAAAACGAAGCAGGGCTTGTGATCGCCGTACAGC |
| | TGAATATTCCCAGCGGCCGGGCTCGCCTCAATGTTTTCACCCCTG |
| | CTCGAGCTAATGTACCCGATTCGACCCCGGTAACGCTGGAAGATT |
| | TCAACGAAGCATTACAAGAAGTCACTGGTGTTGACTACAAGCTCT |
| | CCAACCCTTGCATGCTTGGACGCTTCAACAACGAAGCCCGCTGCG |
| | TCGATACTTACCGGAAGGGACGCATCTTTCTCGCCGGTGATGCAG |
| | GCCACCAGCACCTTCCAGCTGGCGGCCAAGGGCTCAACGTCGGTA |
| | TTCAGGAAGCAACTAACCTTGGATGGAAACTTGGGGCCGTGATTC |
| | GCGGCTATGCTCCGGACTCGCTATTGGATACTTATGAGACCGAAA |
| | GACTTCCAATCGCACAAGGCGTCGTGCAGAGCACCACTGCTCAGT |
| | CTGTTCTCTTTTTTGCGCACAGCGGGCCGGAGTTGGCAATACGAA |
| | GTGTGGTGAACACGTTGCTCAAAATACCAACAGCAAACCACGAC |
| | ATTGCTGTAGGAGTTAGTGGTTTCGGCGTCTCATACCCAAAGCTT |
| | CTTGACATGATACTTCCAGCTGGATGGGAGGCACTTCCAGAGGCG |
| | ATCGCCGGGAAGCGAGCCCTAGATGTGAAGTTACGCGTCGGGAA |
| | TGGCGAGGAAAAGCAGCTAAGCGACTATATGAGGGGAGGGAAGT |
| | GGGTTCAACTTCGGTTCCTTGACAAACTCGCAGGAAGGCCGCCAC |
| | TGCCGGCGTTCGAGGGGGCGACAGAGGTTGTGGATGTCGCTGAA |
| | GTTCTGGAGGGGAAAGGGAGCATGTACCGGGGCGCCTTGAGCGA |
| | ACTGCTAATTCGCCCGGACGGTTATCTAGGGTTTGGAAAGCGCgtat |
| | cgatggattacaaggatgacgacgataagatctgagctcttaattaacaattcttcgccagaggtttggtcaagt |
| | ctccaatcaaggttgtcggct (SEQ ID NO: 73). The capitalized sequence is SEQ |
| | ID NO: 85. |
| pTEF1- | tcaagtttcagtttcatttttcttgttctattacaactttttttacttcttgctcattagaaagaaagcatagcaatctaa |
| AMCJ0100 | tctaagttttaattacaagcggccgcATGACCTACACTACTCATGACGTGGTGATTG |
| 0140.1- | TGGGAGCTGGTCCAGTTGGACTCTTCCTCGCTTGCGAACTACGCC |
| tADH1-m- | TTGCCGGTCTCTCCGTTCTGGTGGTCGAGAAACGAACAAATTCGG |
| pSP-G1 | ACGGCATGGCGGAAACGCGTGCCTTTGTGATGCATGGTCGATCTT |
| (Entry 10 | TGGAAATCTTCGCCTCTCGCGGTCTGCTCGACTCTTTCGTTGAAGC |
| Table 17) | GGGTCAAAAGACCGACTGGTGGCATTACGGTGTTCTCGACACTCG |
| | TCTTGATTACAGTGTTTTCGGCCGTGAAACGGACCAGAACTACGT |
| | GTTACTCGTGCCTCAGTACAAAACCGAGATGATCTTATTTCAACG |
| | CGCCGTGGATCTGGGTGCAGTGATTATCAAGGGTGTCCAGGTCGA |
| | TTCAATACACGAATCCGGGCCTTATGTCGTTGCGAGGGGTTTCTA |
| | CTGCAATAACAAACCTTTCATTGCCAGCGGGAAATACCTCGTCGG |
| | TGCGGACGGTGTTCGCAGCACGATCCGCAAAATCGCCAACATCG |
| | AGTTCACTGGCAATCCACCTGTCAATACAGTCATGAGTGGCGAGG |
| | CTACACTAGGCACGGCCATGCCGAATCCCTACATTGTTCACAACG |
| | AGCACGGCCTAGTTATCGCCGCTGACCTGAGGGTCCCCAGCGGAA |
| | GAACCCGCCTAAATGTGTTCGCGAGTGATCGCGGCACGGTTCCTG |
| | AGTCAGTTGAAGTGACTCTTGAGGAGATGAATCAGAGCTTGCAG |
| | AAAATAACCGGCGTTGACTACAAGCTTTCTAACCCTTGCATGCTA |
| | AAGCGTTTCAGCAACGAACAACGGCTAGCGACAACGTATCGCCA |
| | AAATCGGATTTTCATCGTCGGAGATGCATGCCATAAACACCTCCC |
| | AGCCGGCGGCCAGGGCTTAAATGTCGGTCTCCAAGAAGCTCTGA |
| | ATTTAGGATGGAAACTTGCCGCAGTCATCTCCAAGTCCGCCCCTG |
| | CTTCGTTACTCGATACGTACGAAGAACGATGGCCAGTTGCCAAAG |
| | CTGTTGTACAAAACACAACTTCACAGTCACTCTTATTTTTTGCCTC |
| | GTCTGGCCCAGAATGGGCGGTTAGGGAAGCGATCGACAAGCTTC |
| | TGCGTGTACCAGAGGCCAACAAGCGTTTGGCTAGGGAGATCAGC |
| | GGATTTTCTGTTGCCTACCCCAAATCGCTGGATATGATACTTCCAG |
| | ACGGATGGCGGGCCCTACCAGAGAACATCCAGGGAAAGCGTGCA |
| | TTGAATGTAAAGATGAGATTACCAGATGGGATGGTAACCGAACTT |
| | CGTGATTACACCCAAGACGGACGATGGATCCAGCTGCACCTTCCT |
| | GGAAAGCATATCATTGAACTCCGGCCTCCTCCGGCATTTGGTAAC |
| | TGGACGACGGTAGTGGAAGTTGTCGACATGCCAGATGAAGAAGA |
| | GAAGACGAGCTTGTATATGTGCGGAGTGAGAGAGATGCTGATCC |
| | GCCCGGATGGCTATTTGGCCTTTGGTCGAATGGACGACgtatcgatggat |
| | tacaaggatgacgacgataagatctgagctcttaattaacaattcttcgccagaggtttggtcaagtctccaatca |
| | aggttgtcggct (SEQ ID NO: 74). The capitalized sequence is SEQ ID NO: |
| | 86. |
| pTEF1- | tcaagtttcagtttcatttttcttgttctattacaactttttttacttcttgctcattagaaagaaagcatagcaatctaa |
| KB725976.1- | tctaagttttaattacaagcggccgcATGACTAATCAATATGATGTTGTAATTAT |
| tADH1-in- | TGGAGCTGGGCCAGTTGGATTATTATTGGCTTGTGAATTAGCTTT |
| pSP-G1 | GGGAAAAACAAGCGTCATAGTTCTGGAAAGAGAAGCGTCGCCC |
| (Entry 11 | GTATCTCCGTGGAAAGAAGAACCGACTGGTATGAGAGGTTTGCA |
| Table 17, | CCTGCCTTCTAGTGAAATTTTGTGGCGTAGGGGTTTGTTAGAAAA |
| AL-2-86-I- | AATCTGGACATTGGAGGGTAGACCACATGGCCCGACTAAGACTC |
| C5) | CTGGATTTCAATTTGCAGGCCATTTCGCCGGGATACCTCTTAACC |
| | TATCTCAGCTAGACTTAGATAGGTGGAAATATCGATTGAGGGGT |
| | CCATCATTAATGGGGGGTCCAATAACAATTGCATTAATTGAGAG |
| | AGTGTTAGCAGAACGCGCTGAATCATTGGGAGTCGAAATTCATC |
| | GGGGTTGCGGGTTCGAGAGGATAGTCCAACAATCCGCTGACGGC |
| | GTAACAATAGAAGCGGGTGAAGAAACCCAGACATTTCATGCAA |
| | AATGGCTTATTGGTTGCGATGGCGGTCGTTCCCAAGTCCGTAAG |
| | GCTGCCGAAATCGACTTTCCTGGCACTGACGCGACCTTAACTGG |
| | CTACGTGATCCATTGTGATCTTGACCATCCTGAACGATTGTCACC |
| | TGGTTTCCAACCTACCAAACAGGGAATGTATATTTTTAGAAAGC |
| | CCGGCGCTCTCTACCTGATGGATTTCGACGGTGGTGCCGGCCAA |
| | ACAAGAGAACCAAGCCAGCAGAGACTACAAGAAGTGATGGAGA |
| | GGGTTGTAGGGAGAACTGATATCAAGCTAGAGAAGGTTCACCTA |
| | GCAAGTGCGTTTACGGACAGGGGCAAACAAGCCACAACGTATA |
| | GGAAGGGAAGAGTCCTCTTGGCTGGTGACGCAGCGCACATCCAC |
| | CCACCACTTGGTGCACAAGGTATGAACGCCGGTTTGGGTGATGC |
| | CATGAATCTAGGATGGAAGTTAGCTGCAACCGTTCGTGGAGAAG |
| | AGAAAGGAGTCCGGGCTTTCACCGTGTTGGATACCTATACTTCA |
| | GAGAGACATCCCGTTGGTCAATGGGTCTTAGAATGGAATTACGC |
| | ACAAGTTGCTGCTCTAAAACCCGATGTAACAGGTTACGCCGTAC |
| | AAAAATTAATGAGGGATTTGATTGCAACTGATGACGGTACGAAT |
| | TACTTCATAGATAATGTGTGGGGATTGTCTCAGCGCTATGGTGAC |
| | GGTGAAGGTGTTCATCCAGTAGTTGGAAGATCGGCCCCTGATTT |
| | CACATTTAAAGATGGTAGTAGACTTGGGCCAAAATTAGAGGGTG |
| | GCAGAGGACTTTTTATAGATTTTGAAGATGTGGATCTGGAGAAA |
| | GCAGTTAAGCGATTCGAAAGAGTTGATTATCTCGGACAAAACGT |
| | GGAAGATAGACGTGGAATCAGAGCTTTACTTATTAGACCAGATG |
| | GATTTGTTGCTTGGGCTGTAGAAGAGGGAGATGAACCAGACGCA |
| | GAGGGTTTAGAAACTGAATTAAAAAAGTGGTTTTCTTACgtatcgatg |
| | gattacaaggatgacgacgataagatctgagctcttaattaacaattcttcgccagaggtttggtcaagtctcc |
| | aatcaaggttgtcggct (SEQ ID NO: 75). The capitalized sequence is SEQ ID |
| | NO: 87. |
| pTEF1- | tcaagtttcagtttcatttttcttgttctattacaactttttttacttcttgctcattagaaagaaagcatagcaatctaa |
| AP007159.1- | tctaagttttaattacaagcggccgcATGGGCAAGACCTGGGACGTTATTATTGCTG |
| tADH1- | GCGCTGGGCCGGTCGGTCTTTTCCTCGCCTGTGAACTGGCCATAG |
| in-pSP-G1 | CTGGCGTGTCGGTACTTGTACTGGAGCGAGAGATGCAGCCAGAGT |
| (Entry 12 | CGCCTTGGAAAGAGGGATTGTTTGGGCGTCGCGGTCTCTATACCC |
| Table 17, | CGGCGGTCGAGGCATTTTACCGCCGTGGCATCTTGAAGAGAATTT |
| AL-2-86A- | TCGGTGATGATGAGCGTCCGACGCATTTGAAGAAGACTGAAGGC |
| c1) | TTTCAATATGGAGGACACTTTGCGGGCTTGGTGCTTAATGCAAAC |
| | AACATCGAGTTTTCACGCTGGCCATATCGCTTGCCAGGACCGTCC |
| | TTTCTTCCTGGTCCGACTTCTCTTGGACGTCTTGAGGCTGTGTTGT |
| | CGGAACGTGCAGAGACGCTGGGTGTCCAGATCCTTAGAGGTATG |
| | GAAGTCTCCCGGCTCGCTGATGAAGGTGAATCAGTCAAGGTATGG |
| | GCGGGCGACCAGTGGTTTACGGCTCAATGGCTTGTCGGATGTGAC |
| | GGTGGACGCAGCACAGTTCGGAAAACTGCTGGATTCGAGTTTGTC |
| | GGGACCGAAGCAGAATTCACTGGTTATATTGCGGTATGTGATCTC |
| | GACCGACCAGACTTATTGAAGTCAGGTTTCAAGCACACAAACTCG |
| | GGGATGTACATAGTTAGCGGACCTGGACAGTTGCATGTTATCGAT |
| | TTTGATAGGAGCTTTGATCGGTCACAAACTATCACGCGGGAACAC |
| | TTCCAGGAAGTGCTACGGCGAGTGTCGGGCACAAACATCGTTGTC |
| | GAAGCGCTTCACCTAGCCTCCTCCTTCACCGATCGCTCGAAGCAA |
| | GCAACAGAATATCGTAGAGGACGCATCCTTCTGGCTGGTGACAGT |
| | GCCCACACACACTCTCCACTGGGCGCGCAAGGATTGAGTACAGG |
| | AATTGGCGATGCGATGAATCTCGGCTGGAAGCTTGCGGCTACCGT |
| | CAAGGGGTTTGCATCTCCTGGTCTGCTAGACACCTACCACCAAGA |
| | GCGGCATCCAGAGGCAGCTCGAGTGCTTGAGTGGACTCGCGCTCA |
| | GGTGGCGGCCTTACGCCCTGATCCATATGGCCAGGCTATTGCGAG |
| | TCTCATGCGGGACATGATCAATACCCAAGATGGGGCGACTTATCT |
| | CGCCGATCGCATCTGGGGACTTTCAGTGCGCTATGGCCCGGGCGA |
| | CGCGCATCCACTTGTCGGTTCTAGCGCTCCGGATTTTGAATTCGAT |
| | GATGGAATGCGACTTGGAGCTAAGTTAGAGACAGGATCCTTCTTG |
| | GTGATTGACTTTGGGAGCAACAACCAGGTGGCAGAGCATGTGCA |
| | GTCCTTGCAGTCTCTGCAGTTCATGATTCAATACTGTGCATGTAGC |
| | GCAAAGGAGGAATTCGGGCTCAAGGGGTTACTCTTGCGACCTGAT |
| | GGTGTGGTCGCATGGGTGTCGACGGAGGAGATAAATATAATACG |
| | ACTGCATGTTGCATTGTCTCGTTGGATAAGTCTCCCTAGTTTCGAG |
| | GCTgtatcgatggattacaaggatgacgacgataagatctgagctcttaattaacaattcttcgccagaggttt |
| | ggtcaagtctccaatcaaggttgtcggct (SEQ ID NO: 76). The capitalized sequence |
| | is SEQ ID NO: 88. |

Primers used for OxyS saturation mutagenesis in this Example. To create a saturation mutagenesis of the positions outlined in Table 18 the odd numbered primers were used for PCR amplification with EH414 and the even numbered primers were used for PCR amplification with EH745. The resulting amplicons were gel purified and used for Gibson Assembly with pSP-G1 (empty) digested with SpeI and NotI.

TABLE 16
Primers used for OxyS saturation mutagenesis.

| | | |
|---|---|---|
| EH723 | AGTGCGAGCGTGGACTCCTAGMNNTTTCGAGAAGTCAACAGGCTC (SEQ ID NO: 89) | A43X |
| EH724 | CTAGGAGTCCACGCTCGC (SEQ ID NO: 90) | A43X |
| EH725 | CAATGCATATGGGTGTCTAGTATCAA (SEQ ID NO: 91) | F96X |
| EH726 | CATCATTTGATACTAGACACCCATATGCATTGNNKGTTCCACAAGTACGAACTGAAGAAC (SEQ ID NO: 92) | F96X |
| EH727 | ATGTGGGTCTTGACCTGGAAAATC (SEQ ID NO: 93) | M176X |
| EH728 | AACTATTGGGCATAGATTTTCCAGGTCAAGACCCACATNNKTTTGCTGTCATCGCAGACG (SEQ ID NO: 94) | M176X |
| EH729 | TGCACGAAGGTCATGTCTCATG (SEQ ID NO: 95) | W211X |
| EH730 | TCATGAGACATGACCTTCGTGCANNKTTCGCAGCATTTCCGCTAG (SEQ ID NO: 96) | W211X |
| EH731 | CCATGCACGAAGGTCATGTCTC (SEQ ID NO: 97) | F212X |
| EH732 | GAGACATGACCTTCGTGCATGGNNKGCAGCATTTCCGCTAGAACC (SEQ ID NO: 98) | F212X |
| EH733 | CAGCATACGGTCTATCGAAAAAGGCGACMNNTGCTCTGTAGACGTCTGGTTC (SEQ ID NO: 99) | T225X |
| EH734 | GTCGCCTTTTTCGATAGACCG (SEQ ID NO: 100) | T225X |
| EH735 | CGGCGCCCTCCTGTCA (SEQ ID NO: 101) | V240X |
| EH736 | GTATGCTGACAGGAGGGCGCCGNNKACGGAGGAGGATGTCAGAGC (SEQ ID NO: 102) | V240X |
| EH737 | CTAAGTTCAGTCCCTGGCCACCAGCMNNCAAATGAATATGGCATGCATCACC (SEQ ID NO: 103) | P295X |
| EH738 | GCTGGTGGCCAGGGACTG (SEQ ID NO: 104) | P295X |
| EH739 | ATCTTGAAATCCTAAGTTCAGTCCCTGGCCACCMNNGGGCAAATGAATATGGCATGCATC (SEQ ID NO: 105) | A296X |
| EH740 | GGTGGCCAGGGACTGAACTTAG (SEQ ID NO: 106) | A296X |
| EH741 | TCCTAAGTTCAGTCCCTGGCCMNNAGCGGGCAAATGAATATGGCA (SEQ ID NO: 107) | G297X |
| EH742 | GGCCAGGGACTGAACTTAGGA (SEQ ID NO: 108) | G297X |
| EH743 | TTAACGGCATCTTGAAATCCTAAGTTCAGTCCCTGMNNACCAGCGGGCAAATGAATATGG (SEQ ID NO: 109) | G298X |
| EH744 | CAGGGACTGAACTTAGGATTTCAAG (SEQ ID NO: 110) | G298X |
| EH812 | GTGCGAGCGTGGACTCCTAGAGCMNNCGAGAAGTCAACAGGCTCG (SEQ ID NO: 111) | K42X |
| EH813 | GCTCTAGGAGTCCACGCTCGCACTG (SEQ ID NO: 112) | K42X |
| EH814 | CAACAGTGCGAGCGTGGACMNNTAGAGCTTTCGAGAAGTCAACAG (SEQ ID NO: 113) | G45X |
| EH815 | GTCCACGCTCGCACTGTTG (SEQ ID NO: 114) | G45X |
| EH816 | TGGGTGTCTAGTATCAAATGATGAG (SEQ ID NO: 115) | L95X |
| EH817 | TCTCATCATTTGATACTAGACACCCATATGCANNKTTTGTTCCACAAGTACGAACTGAAG (SEQ ID NO: 116) | L95X |
| EH818 | GGCGACTGTTGCTCTGTAGA (SEQ ID NO: 117) | F228X |
| EH819 | TCTACAGAGCAACAGTCGCCNNKTTCGATAGACCGTATGCTGACA (SEQ ID NO: 118) | F228X |
| EH820 | CATTAATTCCCTTAAACCCTCGTAGCGMNNATCTGGATCAATTAGAACAGCTTGAG (SEQ ID NO: 119) | P357X |
| EH821 | CGCTACGAGGGTTTAAGGGA (SEQ ID NO: 120) | P357X |
| EH822 | AGGATCTGGATCAATTAGAACAGCT (SEQ ID NO: 121) | R358X |
| EH823 | CTCAAGCTGTTCTAATTGATCCAGATCCTNNKTACGAGGGTTTAAGGGAATTAATGATTG (SEQ ID NO: 122) | R358X |
| EH824 | TAGCCCCGCTAAATATCTATTAGTCTCAGGMNNGTGCAACAATTCAATCATTAATTCCCT (SEQ ID NO: 123) | V372X |
| EH825 | CCTGAGACTAATAGATATTTAGCGG (SEQ ID NO: 124) | V372X |
| EH414 | GCTCATTAGAAAGAAAGCATAGC (SEQ ID NO: 125) | |
| EH745 | CTGGCGAAGAATTGTTAATTAAGAGC (SEQ ID NO: 126) | |

This Example describes embodiments involving 6-demethyl-6-epitracycline derivatives of the fungal anhydrotetracycline TAN-1612 in S. cerevisiae. This Example is directed to the product formed by co-expressing the TAN-1612 pathway and the bacterial hydroxylase PgaE. The testing of fungal hydroxylase homologs to DacO1, the 6α-hydroxylase of anhydrodactylocyclinone is then described followed by the description of a method for bacterial monooxygenase saturation mutagenesis near the anhydrotetracycline binding site, with OxyS as an example. The screening process of the natural diversity and generated diversity by mutagenesis is also described. A key result described in this Example is the isolation and characterization of a new major product in a strain co-expressing PgaE and the TAN-1612 pathway that differs from the major product in the strain expressing the TAN-1612 without PgaE (FIG. 22, FIG. 23 and FIG. 25).

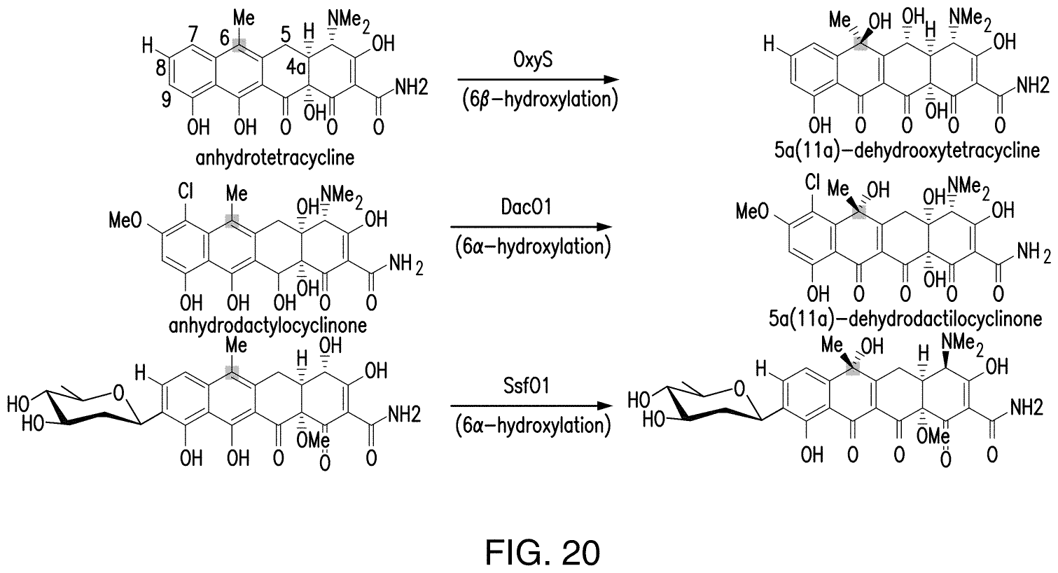

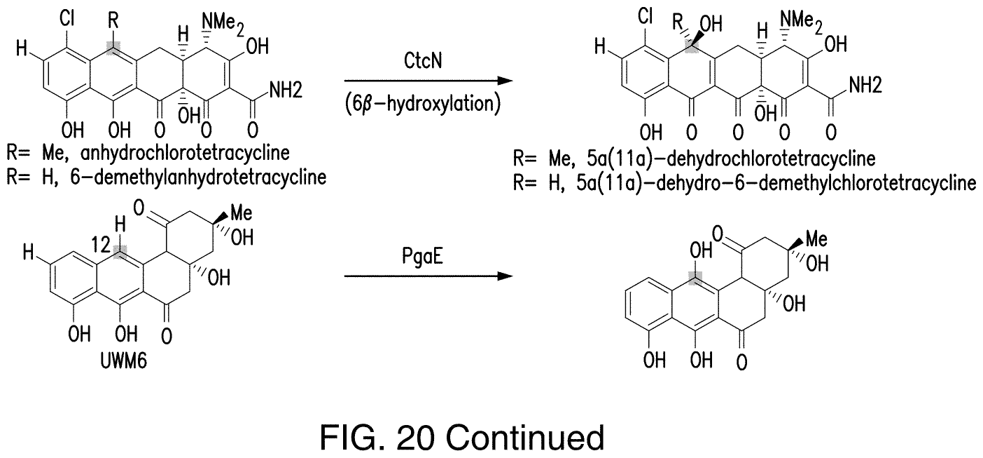

TAN-1612, a fungal anhydrotetracycline derivative originally produced by Aspergillus niger, has been introduced to S. cerevisiae. Examples disclosed herein further improved its titers in synthetic media, thus allowing the heterologous production of TAN-1612 derivatives. TAN-1612 differs from anhydrotetracycline in five functional groups, an A-ring methyl ketone instead of the A-ring amide, a 4α-proton instead of the 4α-dimethylamino, a 4α-hydroxy instead of the 4α-proton, a 6-proton instead of the 6-methyl and an 8-methoxy instead of the 8-proton (FIG. 20). The stereochemistry of TAN-1612 at the 4a, 12a positions has not yet been verified, although the stereochemistry of the product of the homologous fungal polyketide, viridicatumtoxin has been determined to be the same as anhydrotetracycline in these positions. The synthesis of 6-demethyl-6-epiglycotetracyclines is highly desirable. TAN-1612 presents a unique scaffold for the biosynthesizing these derivatives in S. cerevisiae due to its ready biosynthesis in this heterologous host and its unique functional groups among anhydrotetracycline. Much of the knowledge generated in the process of anhydrotetracycline hydroxylation by OxyS and PgaE, as well as DacO1 expression optimization, is directly relevant to the hydroxylation of TAN-1612 in S. cerevisiae as outlined in this example.

Selecting amino acids in monooxygenases binding pocket for mutagenesis. The structure of aklavinone-11 hydroxylase with FAD and aklavinone (PDB ID 3IHG) was loaded on PyMOL. Residues that are within 5 Å from the substrate aklavinone were selected as follows:

PyMOL>hide everything, all

PyMOL>select contacts, (resn VAK and chain A) around 5

Selector: selection “contacts” defined with 88 atoms.

PyMOL>select contacts_res, byres contacts

Selector: selection “contacts res” defined with 268 atoms

PyMOL>show sticks, contacts_res

Testing PgaE and other bacterial hydroxylases for TAN-1612 hydroxylation. Considering the mass spectrometry support for hydroxylation of anhydrotetracycline by OxyS and PgaE it was logical to test them for TAN-1612 hydroxylation (FIG. 19). SsfO1 was tested as well because of its efficient expression in S. cerevisiae and 6α-hydroxylation of its native substrate (FIG. 20).

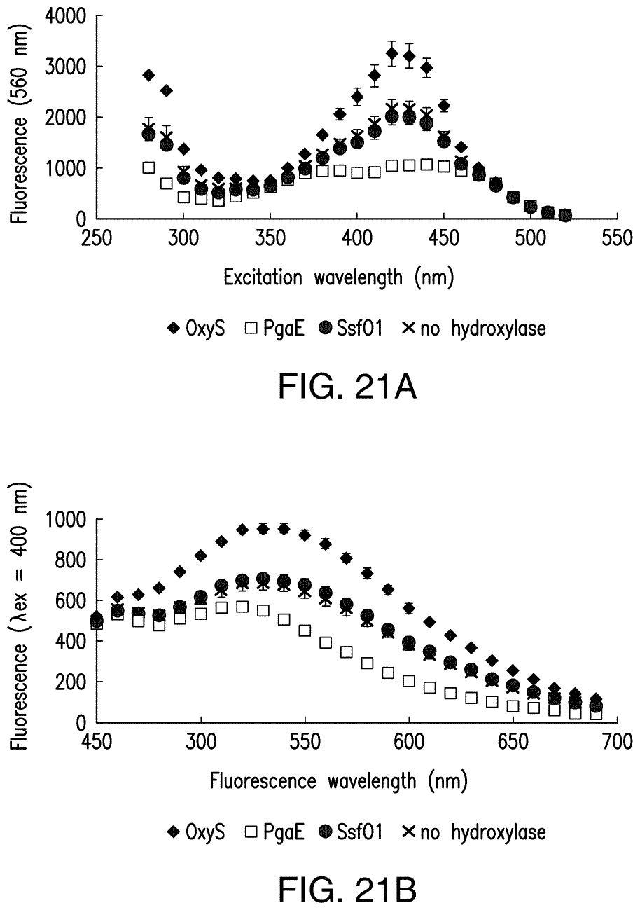

The strain expressing the TAN-1612 pathway without an additional hydroxylase has an excitation peak at 420 nm for 560 nm emission and an emission peak of 530 nm for 400 nm excitation (FIG. 21). This strain was confirmed to produce TAN-1612 as the major compound of 400 nm absorption (FIG. 25A and FIG. 22). The strains expressing OxyS and SsfO1 have similar excitation and emission peaks, although both are increased for the sample encoding OxyS. Notably, the strain encoding PgaE has a significantly reduced emission in 560 nm when excited at 420+/−30 nm (FIG. 25), indicating that TAN-1612 is either not produced or produced in much lower quantities than in the other strains.

To test which compound or compounds are being produced instead of TAN-1612 in the strain expressing the TAN-1612 pathway and PgaE, both that strain and the control strain expressing no hydroxylase were cultured in 500 mL scale to allow purification and further analysis by mass spectrometry and NMR. Following three nights of culturing each culture was extracted twice with EtOAc and the combined organic extract was washed with H₂O and dried with Na₂SO₄ with the organic solvent was removed under reduced pressure. The extract was then purified by semipreparative HPLC prior to NMR and mass spectrometry analysis.

As the absorption chromatogram of the semipreparative HPLC separation shows, the main product with regards to both 400 nm absorption and 254 nm absorption is modified in the +PgaE sample relative to the −PgaE control. While the main product in the −PgaE control elutes after 37 minutes, the main product in the +PgaE sample elutes after 34 minutes (FIG. 22). Interestingly the −PgaE sample has a minor product eluting after 34 minutes, which can be the same product as the main product of the +PgaE sample, or a different one.

The main product of each HPLC separation was isolated and analyzed by NMR and mass spectrometry. The mass spectrometry analysis of the −PgaE compound clearly supports that TAN-1612 was isolated (MS (ES+): m/z calc'd for C₂₁H₁₉O₉⁺, 415.1029; found 415.1026 [M+H]⁺ (FIG. 23A) and the NMR spectrum matches the published TAN-1612 spectrum (FIG. 25A). For the +PgaE sample, the mass spectrum shows four major peaks for an elution peak with absorbance at both 254 nm and 400 nm: 593.1218, 298.0743, 279.0966 and 278.0483 (FIG. 23B). The NMR spectrum of the sample does not show the methoxy and methyl ketone protons of shifts 3.89 and 2.58 ppm as well as the two aromatic protons of chemical shifts 7.05 and 6.45 ppm and does show additional 7 aromatic protons in their stead (FIG. 25B).

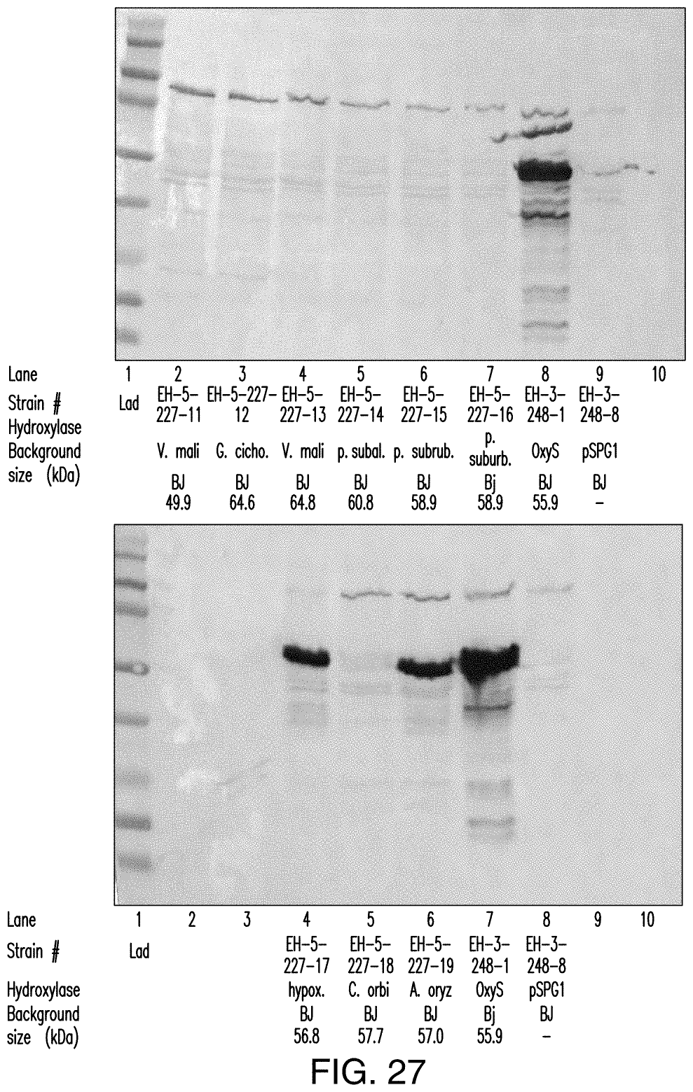

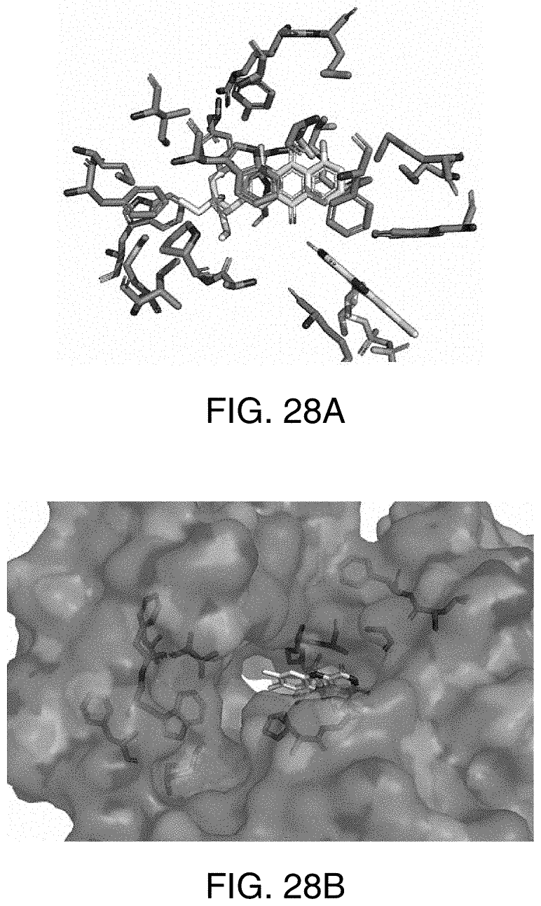

Searching the fungal hydroxylase space for 6α-hydroxylation of TAN-1612. Given the expression challenges with the bacterial monooxygenase DacO1 and given the lack of hydroxylation by codon optimized DacO1 it was logical to test fungal monooxygenases as well. Fungal monooxygenases were chosen based on homology to DacO1 and/or based on the presence of anhydrotetracycline monooxygenase in their CDS product name. Table 17 shows the first twelve such enzymes attempted. Western blot analysis revealed that expression challenges can be prevalent with fungal hydroxylases as well with only, 25% of the fungal hydroxylases assayed displaying a band at the expected size (FIG. 27). The strains that did not indicate any protein expression at the expected size encoded fungal monooxygenases of entries 1, 2, 4, 5, 6, and 11 from Table 17. The two strains that did exhibit protein expression at the expected size encoded fungal hydroxylases Entries 8 and 12 from Table 17 (FIG. 28). For the small sample tested, the two codon optimized fungal monooxygenases did not present a band of the expected size in S. cerevisiae. Two of the six non-codon optimized did presented such a band. The two strains that exhibited a protein of the expected size had a higher identity %, positives % and query cover with both DacO1 and OxyS than the six strains that did not exhibit a band of the expected size but not necessarily in a statistically significant degree (FIG. 27, Table 17).

TABLE 17
Fungal monooxygenases biomined for TAN-1612 6α-hydroxylation in S. cerevisiae.

| | | | | | Identity | Identity |
|---|---|---|---|---|---|---|
| | | | | | (%)/positives | (%)/positives |
| | Gene product | Source | GenBank | Codon | (%)/cover with | (%)/cover with |
| Entry | label | organism | entry | optimized | DacO1 | OxyS |
| 1 | Anhydrotetracycline | Valsa mali strain | CM003104.1 | Y | 30/44/266 | 25/39/388 |
| | monooxygenase | 03-8 | | | | |
| 2 | Anhydrotetracycline | Penicillium | MNBE01000128.1 | N | 32/43/533 | 35/46/541 |
| | monooxygenase | subrubescens | | | | |
| | | strain CBS | | | | |
| | | 132785 | | | | |
| 3 | Anhydrotetracycline | Cercospora | NC_036772.1 | N | 30/41/510 | 34/45/537 |
| | monooxygenase | beticola | | | | |
| | | NC_036772 | | | | |
| 4 | related to | Phialocephala | FJOG01000008.1 | N | 31/46/370 | 30/43/547 |
| | tetracenomycin | subalpine strain | | | | |
| | polyketide synthesis | UAMH 11012 | | | | |
| | hydroxylase tcmG | | | | | |
| 5 | Anhydrotetracycline | Golovinomyces | MCBR01007648.1 | N | 28/43/398 | 30/40/564 |
| | monooxygenase | cichoracearum | | | | |
| 6 | Anhydrotetracyc line | Valsa mali var. | KN714671.1 | N | 23/37/345 | 24/38/405 |
| | monooxygenase | pyri strain | | | | |
| | | SXYL134 | | | | |
| 7 | tetracycline 6- | Aureobasidium | KL584983.1 | N | 26/40/416 | 25/39/455 |
| | hydroxylase protein | pullulans var. | | | | |
| | | pullulans | | | | |
| 8 | Pentachlorophenol 4- | Hypoxylon sp. | KZ111246.1 | N | 34/45/520 | 38/49/531 |
| | monooxygenase | EC38 | | | | |
| 9 | probable 2- | Phialocephala | FJOG01000031.1 | N | 32/46/505 | 39/51/506 |
| | polyprenyl-6- | subalpine strain | | | | |
| | methoxyphenol | UAMH 11012 | | | | |
| | hydroxylase and | | | | | |
| | related FAD- | | | | | |
| | dependent | | | | | |
| | oxidoreductases | | | | | |
| 10 | 2-polyprenyl-6- | Aspergillus | AMCJ01000140.1 | N | 32/47/501 | 34/50/503 |
| | methoxyphenol | oryzae 100-8 | | | | |
| | hydroxylase | | | | | |
| 11 | pentachlorophen ol 4- | Colletotrichum | KB725976.1 | Y | 34/44/531 | 34/47/531 |
| | monooxygenase | orbiculare | | | | |
| | | MAFF 240422 | | | | |
| 12 | unnamed protein | Aspergillus | AP007159.1 | N | 32/45/521 | 34/47/529 |
| | product; 2- | oryzae RIB40 | | | | |
| | polyprenyl-6- | | | | | |
| | methoxyphenol | | | | | |
| | hydroxylase and | | | | | |
| | related FAD- | | | | | |
| | dependent | | | | | |
| | oxidoreductases | | | | | |

Evolving monooxygenases for 6α-hydroxylation of anhydrotetracyclines—OxyS. Considering the effective hydroxylation of anhydrotetracycline in yeast by OxyS, its apparent lack of hydroxylation activity on TAN-1612 and an effective UV/Vis assay for anhydrotetracyclines hydroxylation, directed evolution of OxyS to accept TAN-1612 as a substrate seemed logical. In addition, OxyS structure was previously probed by X-ray crystallography, as well as the structure of a homologous protein, Aklavinone-11-Hydroxylase, along with its native substrate, aklavinone (42% homology, PDB ID 4K2X and 3IHG, respectively). Thus, the substrate binding pocket of OxyS can be identified with some confidence and mutated accordingly.

To perform saturation mutagenesis on residues that are in proximity to the supposed substrate binding pocket of OxyS, residues of Aklavinone-11-Hydroxylase that are within 5 Å of aklavinone were listed. Then the homologous residues in OxyS were noted as well (Table 18). Some of these homologous residues in OxyS indeed sit in proximity to a cavity that is a continuation of the cavity in which FAD is situated in the OxyS crystal structure (FIG. 28).

TABLE 18
Residues chosen for mutagenesis in OxyS.

| | Residue | | | | Distance |
|---|---|---|---|---|---|
| | number in | Residue context in | Residue in | Residue context in | from VAK |
| | 3IHG | 3IHG | OxyS | OxyS | (Å) |
| 1 | R45 | PYPRAAG (SEQ ID NO: 127) | K42 | FSKALGV (SEQ ID NO: 128) | 4-5 |
| 2 | A47 | PRAGQN (SEQ ID NO: 129) | A43 | SKALGVH (SEQ ID NO: 130) | <4 |
| 3 | G48 | AAGQNPR (SEQ ID NO: 131) | G45 | KALGVHAR (SEQ ID NO: 132) | 4-5 |
| 4 | I72 | ADDIRG (SEQ ID NO: 133) | — | | <4 |
| 5 | F79 | QGDFVIR (SEQ ID NO: 134) | — | | <4 |
| 6 | I81 | QGDFVIR (SEQ ID NO: 135) | — | | 4-5 |
| 7 | M101 | DDMVAA (SEQ ID NO: 136) | — | | 4-5 |
| 8 | W113 | PAGWAM (SEQ ID NO: 137) | L95 | PYALFV (SEQ ID NO: 155) | 4-5 |
| 9 | M115 | PAGWAMLSQD (SEQ ID NO: 138) | F96 | PYAL-FVPQV (SEQ ID NO: 156) | <4 |
| 10 | M201 | LTHMVGV (SEQ ID NO: 139) | M176 | --HMFAV (SEQ ID NO: 157) | <4 |
| 11 | W221 | GTTGWYYL (SEQ ID NO: 140) | W211 | DLRAWFAAFP (SEQ ID NO: 158) | <4 |
| 12 | Y223 | TGWYYLH (SEQ ID NO: 141) | F212 | DLRAWFAAFPL (SEQ ID NO: 159) | <4 |
| 13 | T232 | PEFKGTFGPTDRP (SEQ ID NO: 142) | T225X | ATVA (SEQ ID NO: 160) | <4 |
| 14 | P235 | TFGPTDRP (SEQ ID NO: 143) | F228 | TVAFFDRP (SEQ ID NO: 161) | 4-5 |
| 15 | F245 | RHTLFVEYDPDEG (SEQ ID NO: 144) | V240X | RRAPVTEED (SEQ ID NO: 162) | <4 |
| 16 | W287 | VDIQGWEMAARIA (SEQ ID NO: 145) | — | | <4 |
| 17 | M289 | WEMAARIA (SEQ ID NO: 147) | — | | 4-5 |
| 18 | P314 | VTPPTGGMSG (SEQ ID NO: 148) | P295X | IHLPAGGQGL (SEQ ID NO: 163) | <4 |
| 19 | T315 | VTPPTGGMSG (SEQ ID NO: 149) | A296X | IHLPAGGQGL (SEQ ID NO: 164) | <4 |
| 20 | G316 | VTPPTGGMSG (SEQ ID NO: 150) | G297X | IHLPAGGQGL (SEQ ID NO: 165) | <4 |
| 21 | G317 | VTPPTGGMSG (SEQ ID NO: 151) | G298X | IHLPAGGQGL (SEQ ID NO: 166) | <4 |
| 22 | R373 | AIYAQRMAP (SEQ ID NO: 152) | P357 | LIDPDPRYE (SEQ ID NO: 167) | 4-5 |
| 23 | M374 | AIYAQRMAP (SEQ ID NO: 153) | R358 | LIDPDPRYE (SEQ ID NO: 168) | 4-5 |
| 24 | Y387 | SVGYPET (SEQ ID NO: 154) | V372 | LLHVPET (SEQ ID NO: 169) | 4-5 |

Library screening for TAN-1612 hydroxylation in S. cerevisiae. Following the assembly of the libraries of fungal monooxygenases and OxyS saturation mutagenesis the libraries were screened for TAN-1612 hydroxylation using a variant of the microtiter plate assay for anhydotetracycline hydroxylation. As negative control a strain encoding the TAN-1612 pathway without an additional hydroxylase was used. As positive control a strain encoding the TAN-1612 pathway and PgaE was used, as that strain was already shown to produce an alternative major product instead of TAN-1612 (FIG. 22).

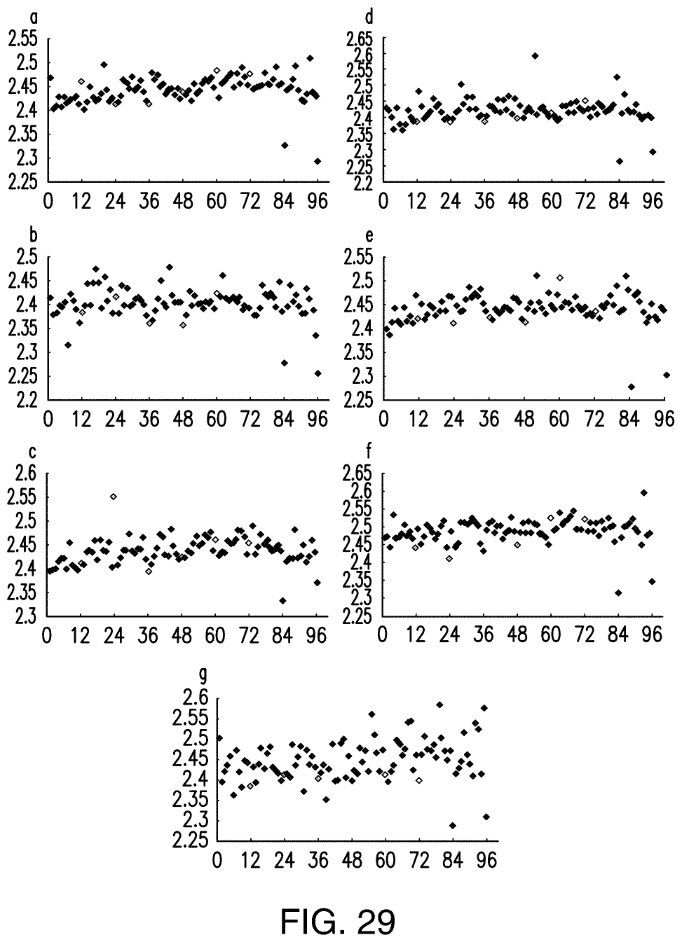

Given that the positive control strain expressing PgaE showed a significantly reduced emission at 560 nm upon 400 and 450 nm excitation, colonies were plotted according to their absorbance at these wavelengths and normalized to OD600: (400+450 nm)/600 nm. When colonies of each 96-well plate were ranked according to this absorption criteria the PgaE positive control was ranked on average 83.6 (out of 96) with a standard deviation of 10.0 (Table 17 and FIG. 29). A low rank of PgaE is expected in the likely scenario that most strains screened do not encode an efficient TAN-1612 hydroxylase. Wells 84 and 96 containing no cells consistently ranked the lowest in the measurement of (400+450)/600 nm (FIG. 29).

TABLE 19
Rank of PgaE positive control in screen for TAN-1612 hydroxylase

| | | Absorbance C1 | Absorbance C2 | |
|---|---|---|---|---|
| Plate | Strain | (400 + 450)/600 | (400 + 450)/600 | |
| a | EH-5-217-2 | 89 | 59 | |
| b | EH-5-217-2 | 91 | 92 | |
| c | EH-5-217-2 | 94 | 68 | |
| d | EH-5-217-4 | 88 | 79 | |
| e | EH-5-217-4 | 78 | 85 | |
| f | EH-5-217-4 | 93 | 87 | |
| g | EH-5-217-1 and | 81 | 87 | |
| | EH-217-3 | | | |
| Average (all) | | | | 83.6 |
| Standard deviation | | | | 10.0 |
| fain | | | | |

Despite the structural dissimilarities between UWM6, the native substrate of PgaE, and TAN-1612 (FIG. 20), it is PgaE and not OxyS or SsfO1 whose co-expression with the TAN-1612 pathway has led to the production of a different major product of 400 and 254 nm absorption instead of TAN-1612. Both the chromatograms for the HPLC separation of the +PgaE and −PgaE samples, as well as the excitation and emission spectra support that PgaE is converting TAN-1612 or an intermediate in its biosynthesis (FIG. 22, FIG. 21). It is therefore of interest to test other enzymes involved in the 12-position hydroxylation of UWM6, such as CabE, LanE, UrdE and GilOI.

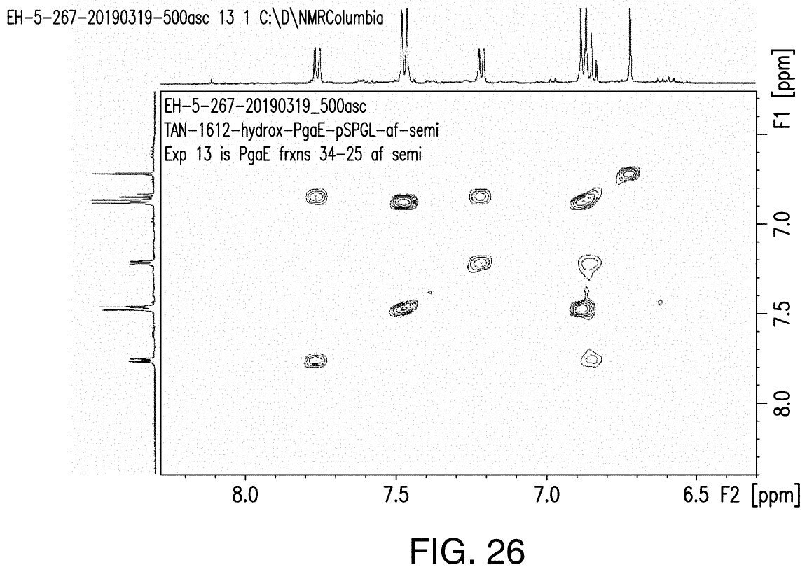

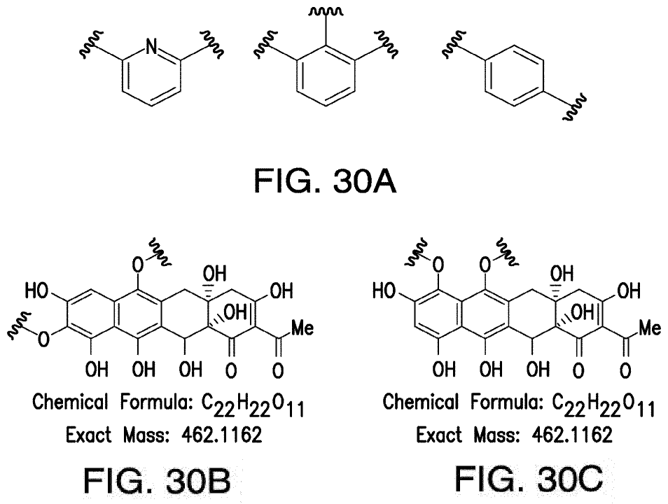

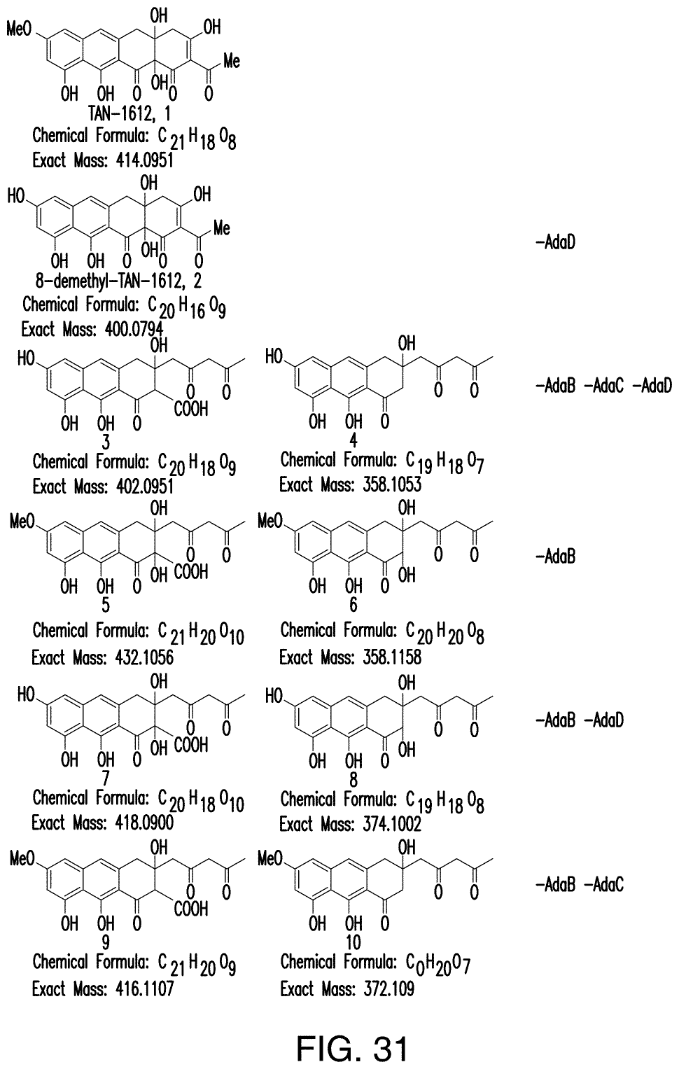

The protons of chemical shifts 7.47 and 6.88 ppm with coupling constant of 8.6 Hz are likely two pairs of equivalent aromatic protons at ortho to each other and they are shown to interact in the COSY spectrum as well (FIG. 25 and FIG. 26); the protons of chemical shifts 7.61, 7.22 and 6.85 ppm are likely three adjacent protons in one aromatic ring that has three other substituents, with the protons of chemical shifts 7.22 and 7.61 ppm likely meta to each other. Interactions between the protons of chemical shifts 7.61 and 6.85 as well as 7.22 and 6.85 ppm are also readily observed in the COSY spectrum and the coupling constants, characteristic of ortho interactions, are 8.0 Hz each (FIG. 25 and FIG. 26). The singlet at 6.72 ppm is likely a single proton in an aromatic ring, possibly the only remaining proton in the D-ring of the substituted TAN-1612 or its intermediate (FIG. 25). The absence of the methoxy shift in the new major product at 3.89 ppm (FIG. 25) can indicate that PgaE derivatization occurs prior to AdaD methylation, which is assumed to be the last procedure in TAN-1612 biosynthesis. It can be that following hydroxylation by PgaE and a potential further derivatization by a yeast endogenous molecule, substrate affinity of AdaD to the modified product is too low to allow methylation. Examples for the moieties that might be part of the newly formed major compound are shown in FIG. 30. Importantly not all moieties are necessarily part of the same molecule, as can be verified by larger scale fermentation followed by further purification and NMR analysis. The aromatic moiety of FIG. 30C can theoretically represent a doubly hydroxylated TAN-1612 or any of the TAN-1612 intermediates of FIG. 31. However, such an assignment is questionable, given the lack of the protons of chemical shift 3.89 and 2.58 ppm corresponding to the methoxy and methyl ketone at the NMR spectrum of the newly formed major compound (compare FIG. 25A and FIG. 25B).

The mass spectrum of the isolated compound from the +PgaE culture has major peaks with m/z values of 593.1218, 298.0743, 279.0966 and 278.0483 (FIG. 21). The 593.1218 ion has an elution peak after 1.60 and after 1.47 min, but an absorption peak at 254 nm and 400+/−60 nm is noted only slightly before the 1.60 min (FIG. 21) and not slightly before the 1.47 peak (the ions are first detected at the diode array before mass spectrometry scanning). Assuming that the two 593.1218 ions can be related stereoisomers of the same exact mass, it is perhaps doubtful whether any of them corresponds to a derivative of the TAN-1612 intermediates shown in FIG. 31. This is because at any stereoisomeric form such compounds can be expected to have absorption at 400 and/or 254.

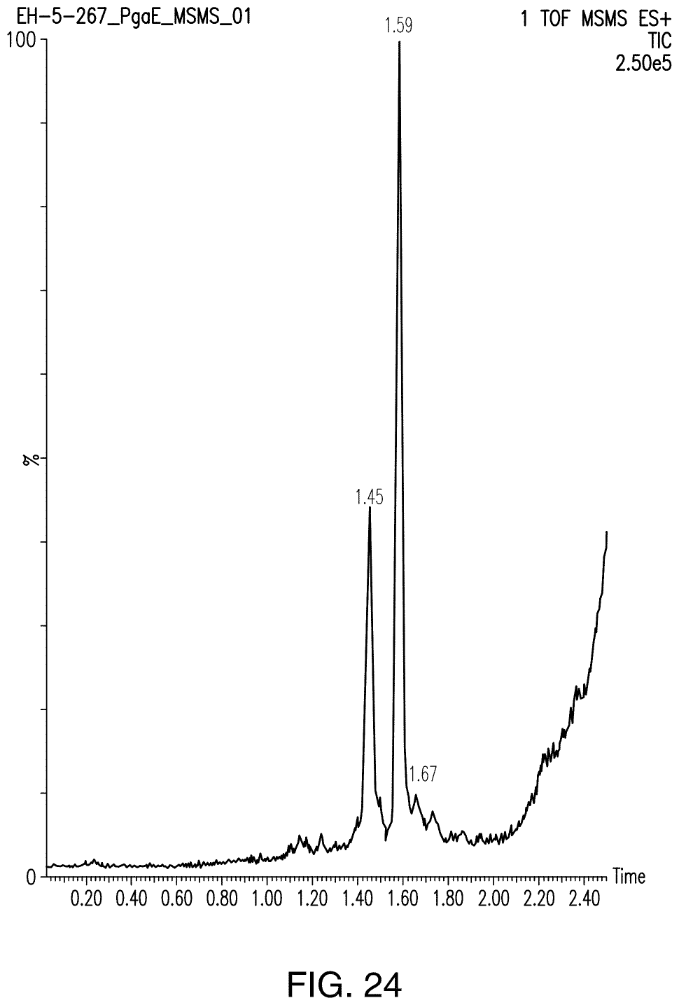

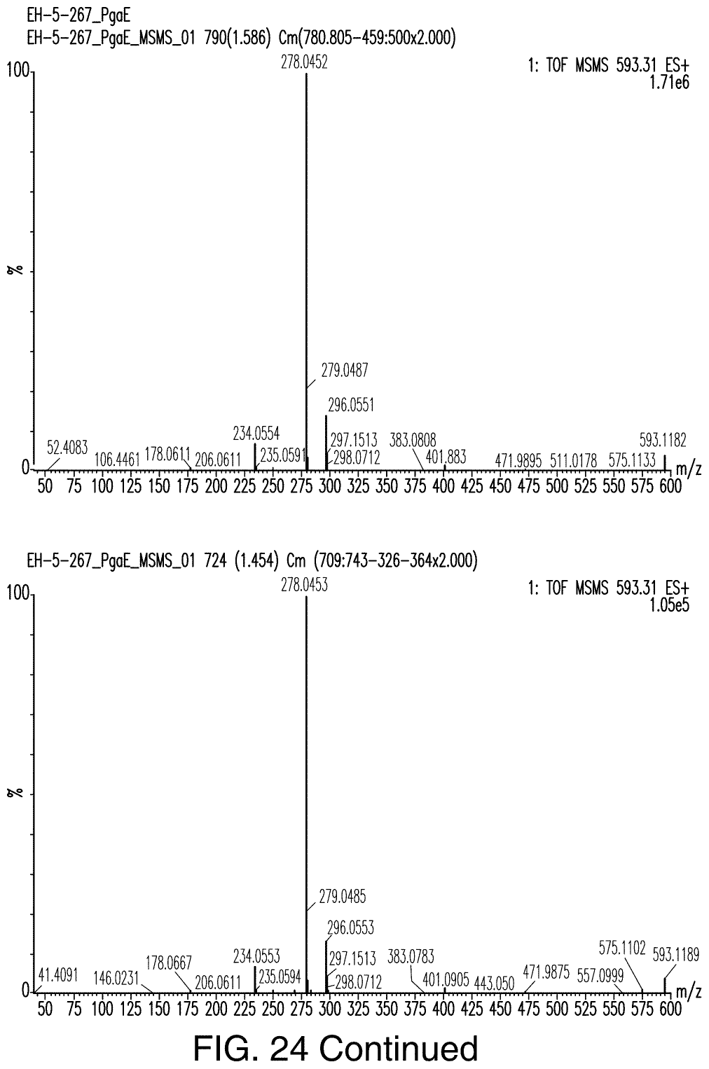

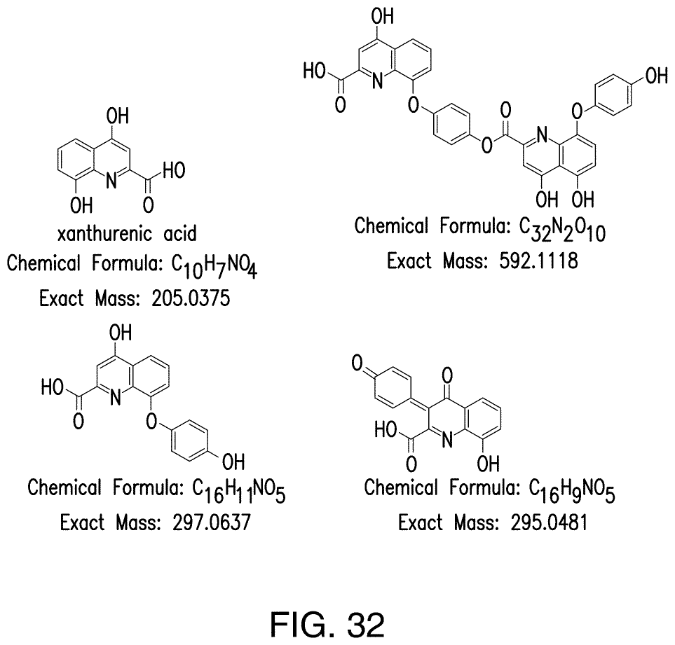

The 298.0743 and 278.0483 ions are three protons and one oxygen different in their m/z values. Possibly a molecule of H₂O+oxidation/reduction apart. In the MSMS spectrum for the ions of m/z 593 both the ions 278.0452 and 296.0551 appear (FIG. 24), the former being the mass of H₂O less than the latter and the latter being H2 away from 278.0483 in its mass. Also of note is that the 593.1218 ion can be a result of a dimer of the 296.0551 ion plus the mass of a proton. Thus, the 593.1218, 298.0743, 296.0551 and 278.0483 ions can correspond to chemical formulas of C₃₂H₂₁N₂O₁₀⁺, C₁₆H₁₂NO₅⁺, C₁₆H₁₀NO₅⁺, C₁₆H₈NO₄⁺ as they differ in 0.0022, 0.0028 and 0.0008 amu, respectively, from the expected masses of ions. These can be ions of some xanthurenic acid derivatives that PgaE might or might not have a contribution to their biosynthesis (FIG. 32). While the derivatives of xanthurenic acid shown in FIG. 32 are not known in yeast, xanthurenic acid is a known yeast metabolite. This proposal of structure or any other structure that involves only yeast metabolites and their derivatives can be explaining the mass and NMR spectra of the isolated major compound in the +PgaE sample (FIG. 22, FIG. 24, FIG. 25 and FIG. 26). However, a major compound that does not involve a derivative of TAN-1612 or its intermediate still does not directly explain how the expression of PgaE leads to the reduction in the TAN-1612 absorbance and fluorescence (FIG. 21 and FIG. 22).

To determine the structure of the newly formed major compound with regards to 254 nm and 400 nm absorption in the +PgaE sample co-expressing the TAN-1612 pathway, the reaction can be repeated in larger scale to isolate enough of the newly formed compound for HSBC and HMBC proton coupled carbon NMR spectra. Furthermore, the minor compound eluting at 34 min for the −PgaE sample is isolated and analyzed as well to identify whether it is identical or at all related to the product isolated from the +PgaE sample that elutes at 34 min (FIG. 22). This characterization can assist in understanding the role PgaE has on the biosynthesis of the newly formed major compound with respect to 400 nm and 254 nm absorption in the +PgaE sample (FIG. 22). Another source of information can be the culturing and isolation of another control strain encoding PgaE but not encoding the TAN-1612 pathway and examining whether such strain displays a similar major peak of in the HPLC chromatogram of 254 and 400 nm absorption as the strain that encodes the TAN-1612 pathway.

Importantly, the library generation approaches used to generate 6α-hydroxylases of anhydrotetracycline can be used for generating TAN-1612 hydroxylases and vice versa. The expression analysis of fungal monooxygenases showed that while branching towards fungal monooxygenases might be a useful strategy to find 6α-hydroxylases and specifically 6α-hydroxylases of TAN-1612, the fungal monooxygenases do not necessarily present an advantage over their bacterial counterparts as far as expression challenges in S. cerevisiae are concerned (FIG. 27).

Amino acids in OxyS were chosen for mutagenesis based on homology to amino acids in the proximity of the substrate in the crystal structure of RdmE and this approach can be implemented in other monooxygenases (or other biosynthesis enzyme), such as PgaE or expression optimized DacO1. Importantly, implementing this approach does not require an existing crystal structure of the monooxygenase of interest. Also, as seen in the case of OxyS, at least some of the amino acids actually sit in proximity to the anhydrotetracycline cavity of OxyS (FIG. 28). However, in the case of OxyS, an approach that uses the published crystal structure of OxyS to choose additional amino acids for mutagenesis or to avoid mutating some of the amino acids that are not in proximity to the cavity might have been preferred. Further mutagenesis is being performed to explore the effect of additional mutations of OxyS in positions that are homologous to amino acids in RdmE structure that are up to 8 Å from aklavinone in 3IHG. Similar mutagenesis studies on stably expressed DacO1 fusion proteins, as well as PgaE are directly relevant for testing 6α-hydroxylation of TAN-1612.

In the screen for TAN-1612 hydroxylation, a variant of the microtiter plate assay for anhydrotetracycline hydroxylation described in Example 2 was used. Example 2 also discusses non-microtiter plate avenues for the anhydrotetracycline hydroxylation, and these can be especially interesting to examine in the context of TAN-1612 hydroxylation, as TAN-1612 does not need to be exogenously supplied to the cells.

Similar to the +PgaE+TAN-1612 encoding strain, strains with a lower (400+450)/600 nm absorption than the PgaE positive control (FIG. 29) are assayed for TAN-1612 hydroxylation by UV/Vis and mass spectrometry. If data supports production of an alternative major product to TAN-1612 these strains are cultured in larger scale to allow purification and analysis of the product by NMR. If data does not support TAN-1612 hydroxylation in any of the assayed strains, strain selection for further assay is attempted based on other criteria such as 550+600−450 nm emission upon 400 nm excitation or 400+450−350 nm excitation for 560 nm emission. These criteria are based on the reduced emission and excitation peaks observed for the PgaE encoding positive control strain (FIG. 21).

This Example describes the progress towards TAN-1612 hydroxylation in S. cerevisiae, the first procedure in the synthesis of 6-demethyl-6-epitetracycline from TAN-1612 in S. cerevisiae. This example shows that a strain expressing both PgaE and the TAN-1612 pathway produced a major compound with regards to 400 nm and 254 nm absorption that differs from TAN-1612 (FIG. 21, FIG. 22, FIG. 23 and FIG. 25). TAN-1612 hydroxylation in S. cerevisiae can also include a larger scale culturing of the +PgaE +TAN-1612 pathway strain to for NMR testing. In addition, the minor compound eluting at 34 min for the −PgaE sample (FIG. 22) is isolated and analyzed to verify whether it is identical or at all related to the product isolated from the +PgaE sample that elutes at 34 min. Another control strain encoding PgaE but not encoding the TAN-1612 pathway are cultured and examined to discover whether it displays a similar major peak in the HPLC chromatogram to that of the +PgaE+TAN-1612 pathway strain.

This Example also describes a hydroxylase harvesting and mutagenesis strategy as well as a UV/Vis assay as a platform for identifying TAN-1612 hydroxylation. This platform was applied to the screening for TAN-1612 hydroxylation by OxyS mutants, DacO1 fusion proteins, and other hydroxylases from bacterial and fungal sources. Further culturing and spectroscopic analysis can be done on strains co-expressing the TAN-1612 pathway and one of the above-mentioned hydroxylases, identified as potentially hydroxylating TAN-1612 in the UV/Vis assay (FIG. 29). In addition to PgaE, and the other hydroxylases described herein, other enzymes involved in the 12-position hydroxylation of UWM6, such as CabE, LanE, UrdE and GilOI can also be considered. With a mutagenesis strategy that does not require a crystal structure that can be used for DacO1 (FIG. 28) and with the additional benefit of the PgaE crystal structure, mutagenesis studies, such as shown on OxyS and on DacO1, on stably expressed DacO1 fusion proteins, as well as PgaE can be beneficial for 6α-hydroxylation of TAN-1612.

Identification and overexpression of a reductase responsible for 5a(11a)-dehydrotetracycline reduction in S. cerevisiae is potentially useful for the reduction of the hydroxylation product of TAN-1612 as can be other efforts in the reduction of 5a(11a)-dehydrotetracycline reduction in S. cerevisiae. Finally, anhydrotetracycline hydroxylations can rely heavily on the UV/Vis assay developed for anhydrotetracycline hydroxylation. More broadly, medium- and high-throughput assays are of very high importance to small molecule production in microorganisms because of the inability to predict in advanced the successful strain modifications needed in the complex environment of the cell. TAN-1612 and anhydrotetracycline hydroxylation belong to a special group of biochemical transformations that are easy simple to assay in high throughput because of the inherent change in the chromophore of the CD ring (FIG. 20). However, to synthesize 6-demtehyl-6-epiglycotetracyclines from TAN-1612 require a general, readily implemented, high-throughput assay for tetracycline biosynthesis in yeast. The development and characterization of such an assay is described in Example 7.

### Example 4. Generating Biologically Active Tetracycline Analogs of TAN-1612 for Tetracycline Discovery

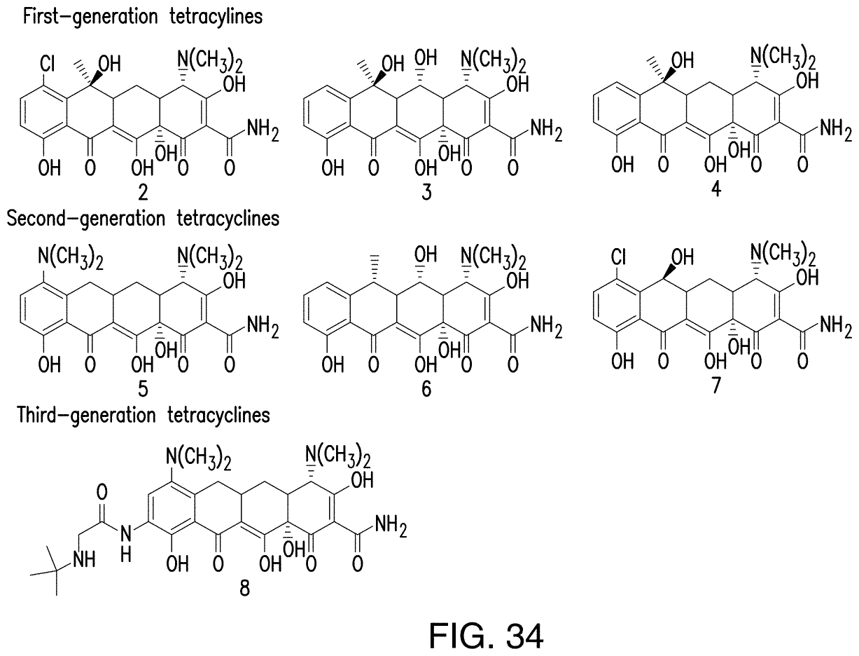

Small changes in the tetracycline structure can lead to major and distinct pharmaceutically essential improvements. Examples include tetracycline-analogs differing from tetracycline in only up to three positions and showing improved pharmacokinetic properties, binding affinity to the ribosome, activity against resistant strains and non-antimicrobial properties. For example, doxycycline has improved half-life, tissue penetration and matrix metalloproteinase (MMP) inhibition and minocycline has an improved pharmacokinetic profile, anti-inflammatory properties and neuroprotective properties compared to tetracycline (FIG. 34). Tetracycline semisynthesis from bacterial fermentation products can yield modifications in positions 2, 4, 5, 6, 7 and 9 and despite the limitations in the positions that can be modified and limitations in the functional group that can be introduced, this strategy yielded all 5 FDA-approved tetracyclines that are not natural products. More recently, a breakthrough in tetracycline total synthesis permitted tetracycline analogue modifications previously impossible, particularly in the D-ring and yielded two 7-dedimethylamino-7-fluoro-9-amidominocycline clinical candidates.

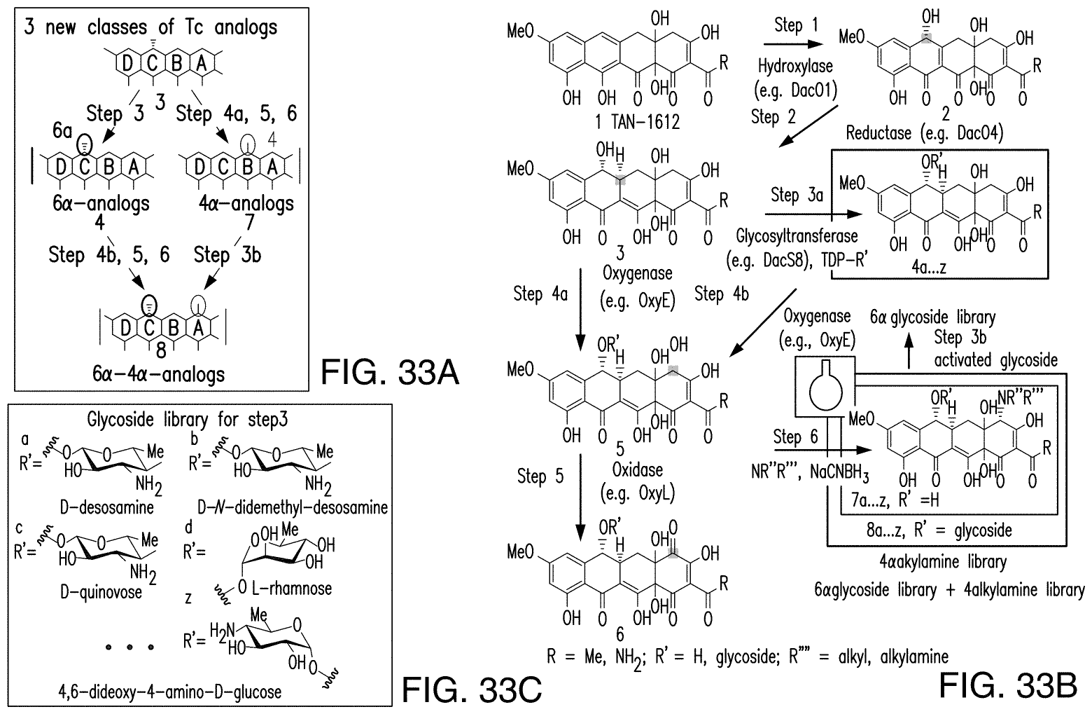

Unlocking unattainable tetracycline analogue chemistry is urgently required to deliver new tetracycline therapeutics and this effort depends upon novel scaffolds and innovative synthesis strategies. The fungal tetracycline TAN-1612 was previously identified as a unique scaffold to access functional group diversity in the 6α, 4a and 4α positions that show promising potential for generating new antimicrobials that cannot be accessed by other routes. The modularity of the biosynthetic/semisynthetic platform, that decouples high-titer scaffold biosynthesis and enzymatic/chemical derivatization, allows a combinatorial testing of 6a, 4a and 4a modifications to explore three new classes of tetracyclines (FIG. 33A). Further, it allows key SAR questions to be studied, such as whether installing a glycosamine on the 6a position of tetracyclines increases antibiotic activity for analogs modified in the 4a position.

Specifically, there is great promise for the 6a position in producing a library of 6-demethyl-6α-glycotetracyclines that builds on the potential and eliminates the major disadvantages of dactylocyclines. A few dactylocyclines are the only known 6α-glycotetracyclines and they proved active against tetracycline-resistant Gram-positive strains. However, their acid sensitivity and their ineffectiveness against gram-negative strains necessitates the generation of analogs. While the modification of the 6a position is emphasized, introducing a large diversity to the 4a and 4a positions by semisynthesis is anticipated (FIG. 33).

Obtaining a library of 6-demethyl-6α-glycotetracyclines from TAN-1612 requires three procedures: (i) hydroxylation of TAN-1612 in the C6 position, yielding a hydroxy handle that is crucial to generate a library of 6α-derivatives in (iii) below; (ii) reduction of the C6-hydroxylated TAN-1612 at the 5a(11a)-enone that is needed since tetracyclines are generally more antibiotically active and more stable than their 5a(11a)-dehydrotetracyclines counterparts and (iii) enzymatic glycosylation of the C6 hydroxy handle introduced in (i) with a library of activated glycosides.

Procedures (i) and (ii) above were implemented in yeast relying on the functional screening of genome-mined hydroxylases and reductases from various organisms with known tetracycline C-ring activity. The desired enzymatic properties, such as flexibility for non-natural substrates, catalytic rates, and expression levels are optimized by directed evolution using the analog-specific FP and Y3H assays. In (iii), a set of known, genome-mined and laboratory evolved promiscuous glycosyltransferases are used for the production of a library of glycosides analogs.

Specifically, for (i) (FIG. 33B) the FAD-dependent hydroxylase OxyS, previously studied in vitro and in bacteria. In preliminary studies, results in S. cerevisiae for functional expression as well as Mass Spectrometry (MS)-verified hydroxylation of Atc, the commercially available proxy for TAN-1612 were obtained. The functionality of less-studied OxyS homologs, such as DacO1, PgaE, SsfO1 and CtcN, initially using an MS-based screen are screened.

Specifically, for (ii) (FIG. 33B), the DacO4-reductase family, a bacterial F420-dependent reductase that performs the analogous 5a(11a) reduction in the dactylocycline pathway is employed. In preliminary results stable expression of DacO4 and its homolog OxyR in S. cerevisiae was shown (data not shown). In analogy to (i), enzymatic functionality of DacO4, OxyR and a set of their genome-mined homologs, such as TPA0598_07_00750 from the marine-derived Streptomyces sp. TP-A0598 and CtcM from Kitasatospora aureofaciens was screened by MS. Members of the Old Yellow Enzyme (OYE) family, a protein family that was found to be highly promiscuous, catalyzing enone reduction to ketones in a variety of substrates are screened. OYEs do not require F420, a cofactor non-natural to yeast, as a cofactor. Towards the biosynthesis of Fo, a functional alternative to cofactor F420, is co-expressed in the v2.0 producer an Fo synthase from Chlamydomonas reinhardtii. As an alternative, the strains can be provided with Fo synthesized by a 6-part convergent synthesis.

Specifically, for (iii) (FIG. 33B) the glycosyltransferase DacS8 from the dactylocycline biosynthetic pathway of Dactylosporangium sp. 14051 (ATCC 53693) and a diverse set of glycoside biosynthesis enzymes are employed. As an example, the manipulation of the well-studied desosamine biosynthetic pathway, including targeted deletions in the well-studied DesI-DesVII and heterologous expression of additional glycoside biosynthetic genes such as CalH, StrL and StrM yielded the glycosides of analogs 8a-f (FIG. 33A-C). As an alternative to performing the glycosyltransferase procedure in yeast (3a), it is performed in vitro (3b). Thus, the Snyder team installs a diverse activated glycoside library and other electrophiles on the C-ring hydroxy handle of 7 (FIG. 33B).

In a parallel approach, 4α-analogs are employed both as a separate library and in combination with the 6α position (FIG. 33A). Firstly, a library of amines in the 4α position semisynthetically from 6 by reductive amination with a corresponding library of secondary amines is produced. This semisynthetic approach would enable the exploration of an expansive library of tetracyclines derivatized with diverse unnatural amines. In analogy to the 6a position, the 4a needs to be prepared for derivatization in two enzymatic procedures: hydroxylation and oxidation (FIG. 33A-C). These procedures are performed by OxyE and OxyL from the oxytetracycline pathway of S. rimosus evolved for TAN-1612 specificity by employing the Y3H FACS with a TetR evolved for binding 15 and 16, respectively.

Towards enzymatic production of 2-deacetyl-2-carboxamido TAN-1612 analogs (R=NH₂, FIG. 33B) to enhance ribosomal binding, and as a result antibiotic activity, (i) encode the genes are encoded to produce the malonamoyl CoA precursor, and (ii) the Ada enzymes are evolved to accept the unnatural substrate or as an alternative, and (iii) the corresponding homologous enzymes are employed from the viridicatumtoxin pathway. Thus, the 2-carboxamido are incorporated by encoding oxyD and oxyP from the oxytetracycline pathway of S. rimosus in the TAN-1612 S. cerevisiae producer strain.

As an alternative, VrtJ, VrtB and VrtA the fungal polyketide viridicatumtoxin are used. In either case the Y3H system is used for tetracycline analogs to assay mutants of the TAN-1612 pathway enzymes for relaxed substrate specificity towards the new 2-carboxamido intermediates. Lastly, the antimicrobial activity of the novel analogs is tested against key clinically important tetracycline-resistant isolates of Streptomyces aureus, Streptomyces enterica, pathogenic Escherichia coli.

### Example 5. Synthesizing 6-demethyl-α-6-glycotetracyclines in Yeast

Tetracyclines are a major class of antibiotics that were discovered in the 1940's. It has been used as broad-spectrum antibiotics, as well as for other types of disease, such as periodontitis. The mechanism includes inhibit bacterial protein synthesis by binding reversibly to the 30S ribosomal subunit and sterically hindering aminoacyl-tRNA binding to the ribosomal A-site. But resistance to tetracyclines include efflux pumps, ribosomal protection proteins, rRNA mutations, enzymatic degradation, etc.

Key FDA approved tetracycline natural products and semisynthetic analogs include chlortetracycline, oxytetracycline, tetracycline, minocycline, doxycycline, demeclocycline and tigecycline (FIG. 34).

Tetraphase approach to tetracycline analogs is a route towards some key analogs. A new synthetic route can lead to new analogs, mostly in the D-ring.

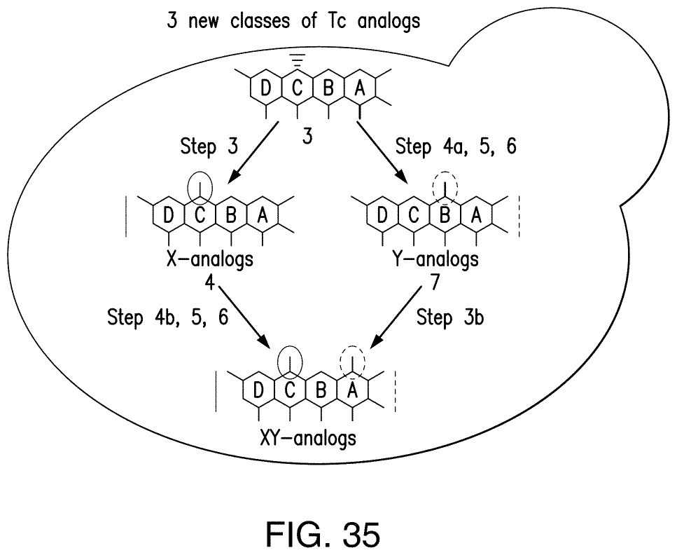

Tetracycline analogs in yeast is a combination approach. Yeast metabolic engineering (ME) can make new analogs inaccessible by total synthesis and semisynthesis (FIG. 35).

Non-antibiotic properties of tetracyclines are many. Natural products have privileged scaffolds. e.g. interaction with proteins. Advanced intermediates can be used as substrates biosynthetic enzymes. Interaction of the final products with target proteins produces natural products, which are useful as drug candidates or lead structures. Tetracyclines are much promise as anti-bacterial and beyond; they were approved periodontitis, tested anticancer, anti-inflammatory. Tetracycline analogs are developed for improved antibacterial. Some are also improved non-antimicrobial (e.g. minocycline). More tetracycline analogs are needed for potentially better anti-inflammatory agents, anti-cancerous agents, MMP inhibitors, etc. It is better to use a non-antibiotic tetracycline for a non-antibiotic application to prevent overuse of antibiotics with less antibiotic resistance and to preserve microbiome balance. Doxycycline is the only FDA approved MMP inhibitor (periodontitis).

Table 20 compares some tetracycline analogs. “Modifications at the C6 atom have produced by far the greatest success in evolving highly active tetracyclines.” “C6 monosubstituted tetracyclines are more [antibacterially] active when substitution takes place at the α-position than compounds substituted at the β-position.” (Table 21).

TABLE 20
Reasons to pursue α-6-glycotetracyclines.

| | 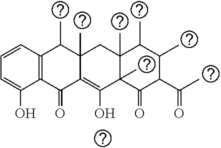 | 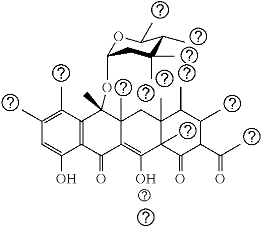 | 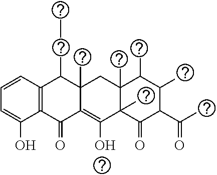 |
|---|---|---|---|
| Source | Synthetic | Natutal product | Heterolog, biosynth. |
| Activity against Tc-sensitive | ✓ [symbol] | ✓ [symbol] | ✓ [symbol] |
| gram (+) strains | | | |
| Activity against Tc-resistant | ✓ [symbol] | ✓ [symbol] | ✓ [symbol] |
| gram (+) strains | | | |
| Activity against gram (−) | X | X | ✓ [symbol] |
| strains | | | |
| in vivo efficiency | X | X [symbol] | ✓ [symbol] |
| Add stability | ✓ | X | ✓ [symbol] |

[symbol]

indicates data missing or illegible when filed

Bulky 6β-substituents destabilize the essential lipophilic form. One reason to make glycotetracyclines that are 6-demethyl is that tertiary aglycones are too acid labile (e.g. dactylocyclins: mild r.t. acid hydrolysis; pH<4) (Table 21 and Table 22)

TABLE 21
Reasons to make glycotetracyclines that are 6-demethyl.

| | 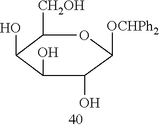 | 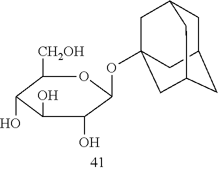 | 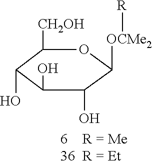 |
|---|---|---|---|
| Electronic enhancement of acid hydrolysis | ✓ | X | ✓ |
| Steric enhancement of acid hydrolysis | X | ✓ | ✓ |
| Relative rate of acid hydrolysis (to OMe) | 25.6 | 48.5 | 557 (6) 31,000 (36) |

TABLE 22
Accessibilities of different synthesis methods to
different tetracycline analogs.

| | Total | Semi | Heterolog. |
|---|---|---|---|
| | synthesis | Synthesis | biosynthesis |
| Access to 6-demethyl-α-6- | X | X | ✓? |
| hydroxy derivatives | | | |
| Access to 6-demethyl-β-6- | X | ✓ | ✓? |
| hydroxy derivatives | | | |
| Access to tertiary α-6-hydroxy | X | ✓ | ✓? |
| derivatives | | | |
| Access to tertiary β-6-hydroxy | ✓ | ✓ | ✓? |
| derivatives | | | |

6α-glycotetracyclines are inaccessible through total synthesis. 6α-tetracyclines only Me, Ar, no heteroatoms while for 6β-tetracyclines, there is no convergent approach.

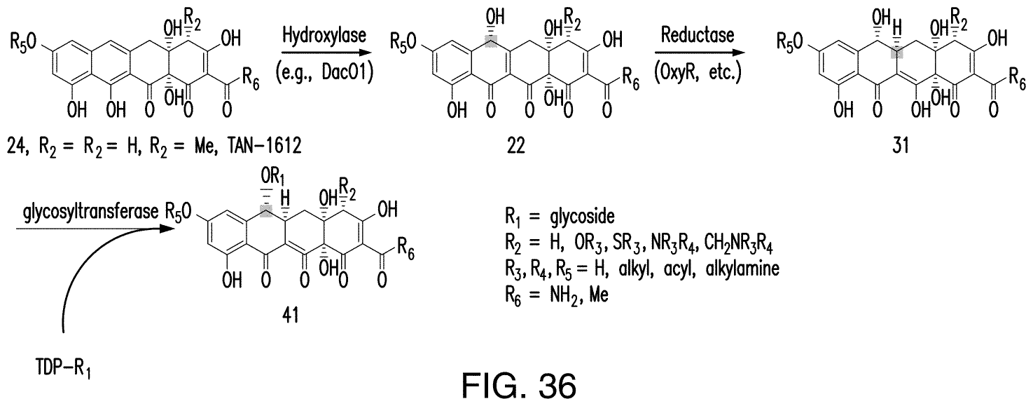

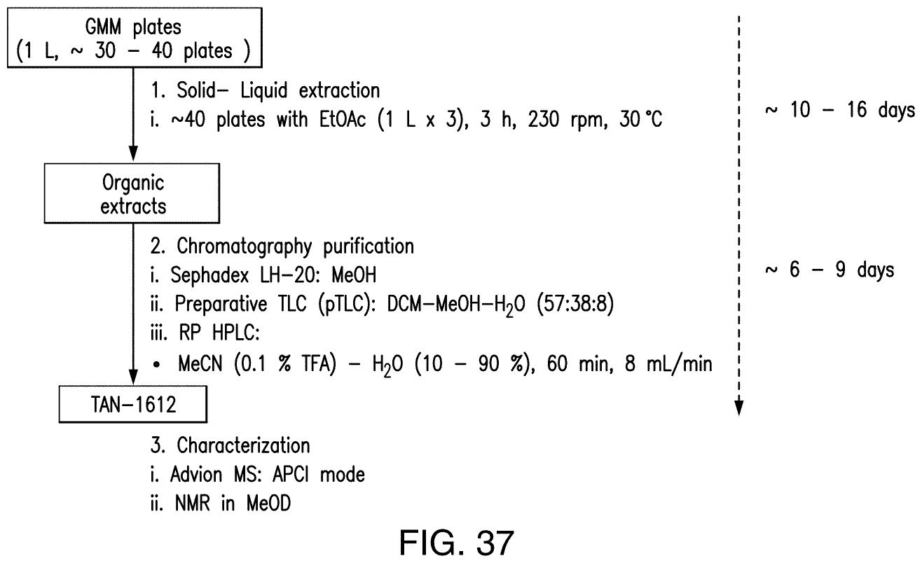

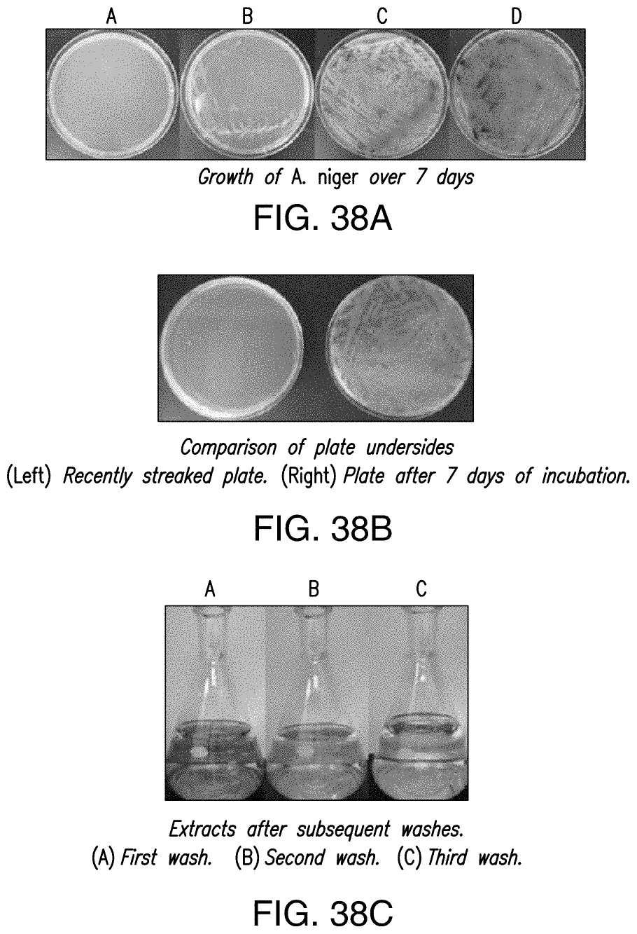

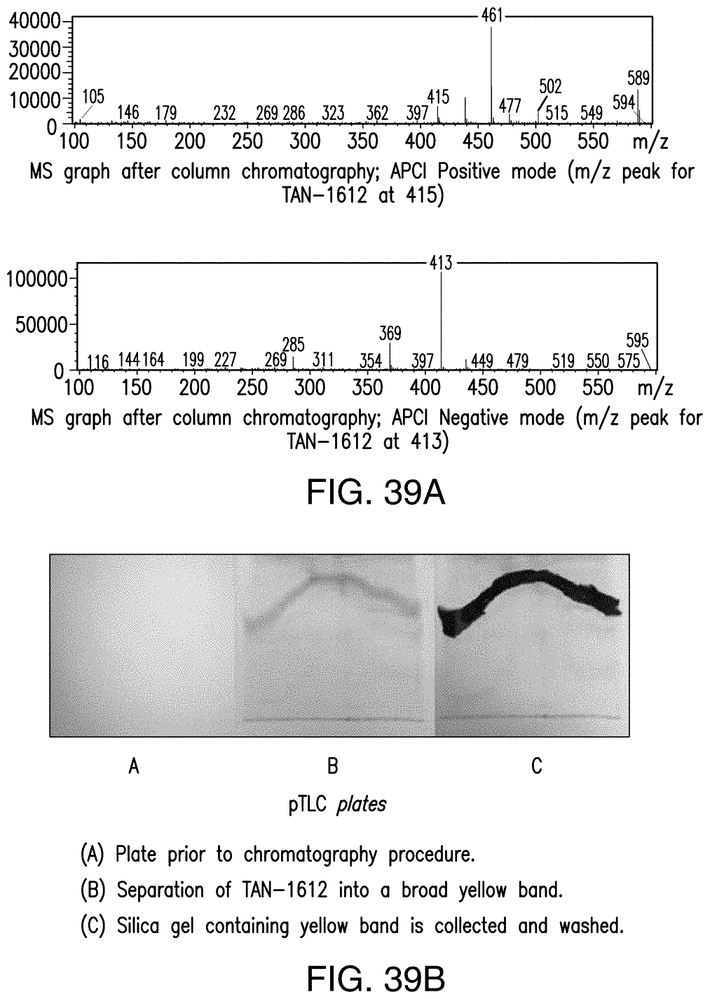

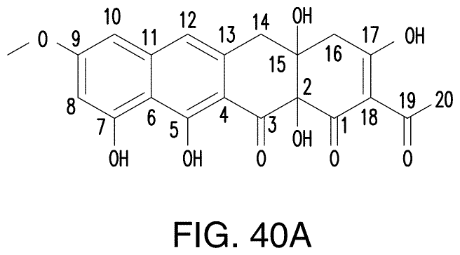

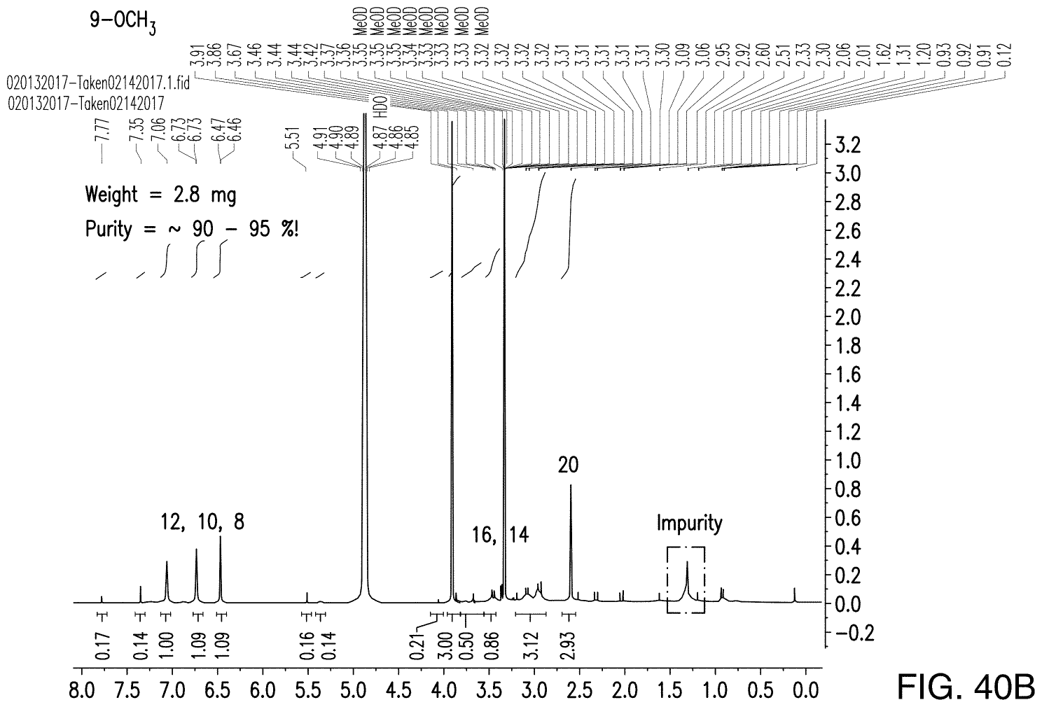

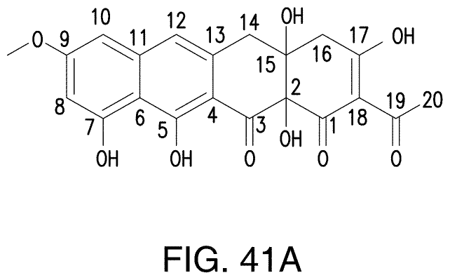

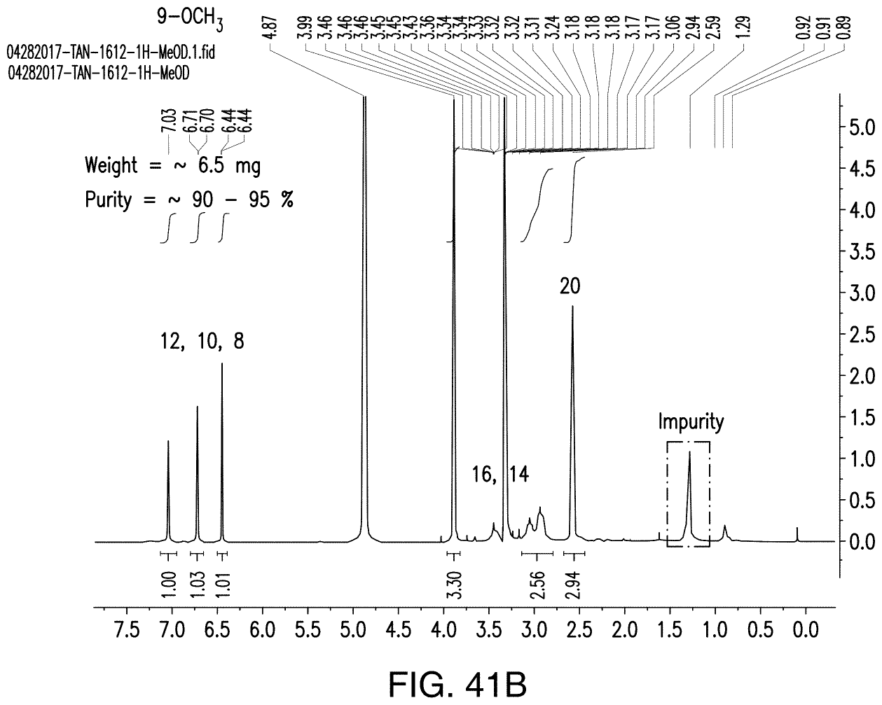

TAN-1612 is used to produce glycotetracyclines. Three procedures include (1) Hydroxylase—6α-hydroxy handle, (2) Reductase—5a,11a, and (3) Glycosyltransferase. (FIG. 36).

### Example 6. TAN-1612: Metabolic Engineering Approaches to Increase Biosynthetic Titers in Yeast

The following methods were used in the Examples disclosed herein.

Strains. The TAN-1612 yeast producers are based on the parent strain—Saccharomyces cerevisiae derived from strain BJ5464 obtained from ATCC (Saccharomyces cerevisiae Meyen ex E.C. Hansen (ATCC®208288™).

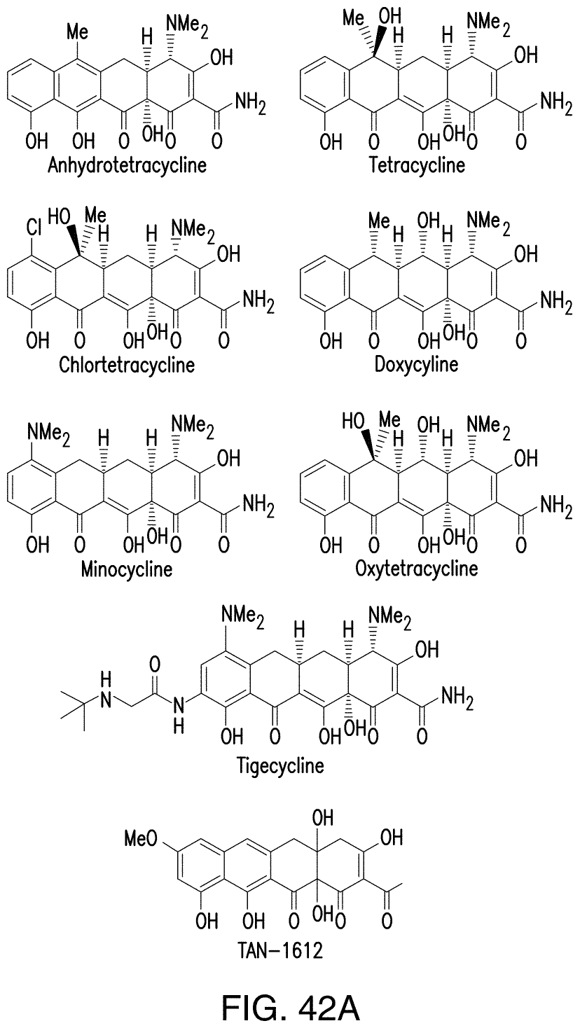

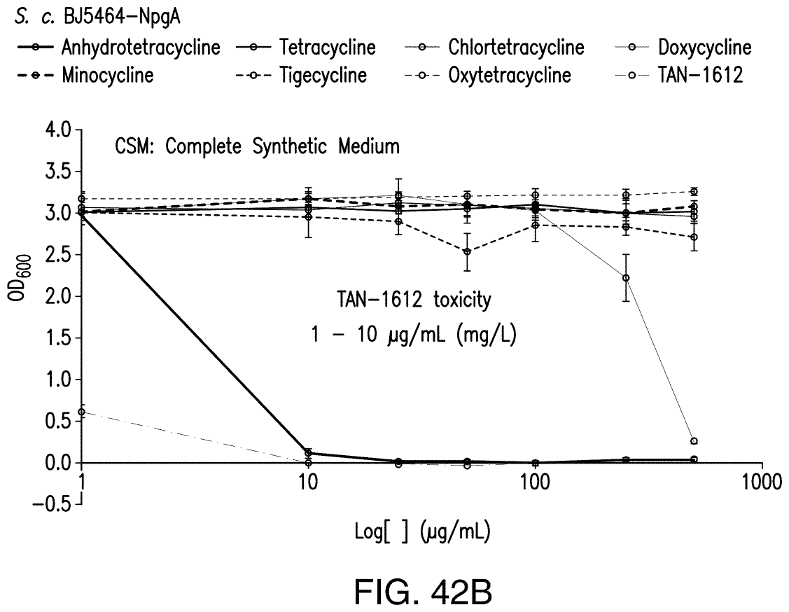

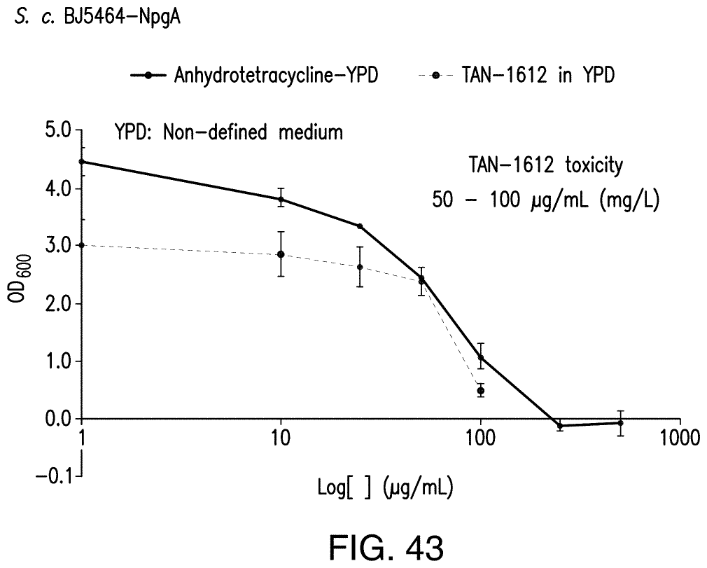

The toxicity assay of TAN-1612 in Saccharomyces cerevisiae. BJ5464 strains were cultured in complete synthetic medium in presence of different tetracycline or its analogs. TAN-1612 exhibited toxicity at the range of 1-10 μg/ml (mg/l). (FIG. 42B) BJ5464 strains were cultured in YPD: non-defined medium in presence of different tetracycline or its analogs. (FIG. 42A). TAN-1612 exhibited toxicity at the range of 50-100 mg/l. (FIG. 43).

Genome Mining of Efflux Pumps in A. niger. Genome mining of efflux pumps in A. niger led to the identification of a supposed TAN-1612 pump (ASPINDRAFT 48051) within its biosynthetic gene cluster.

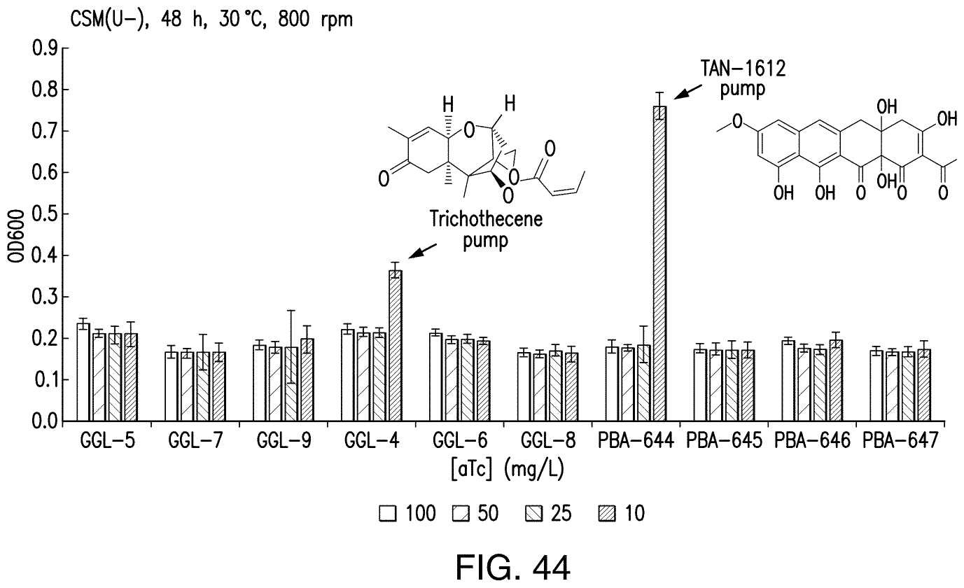

Efflux pump approach. Four different efflux pumps from A. niger (ASPINDRAFT 176833, 185231, 43349 and 48051) were tested their abilities in reducing the toxicity introduced by TAN-1612. Four different efflux pumps from A. niger (ASPINDRAFT 176833, 185231, 43349 and 48051) were constructed and expressed in BJ5464. Sequences for the different efflux pumps are provided in Table 1.1. Cell growth assay (OD600) in the presence of anhydrotetracycline (TAN-1612 analogue) was performed. (FIG. 44). Both trichothecene pump and TAN-1612 pump reduced the toxicity of TAN-1612 analogue with TAN-1612 exhibiting a more robust reduction.

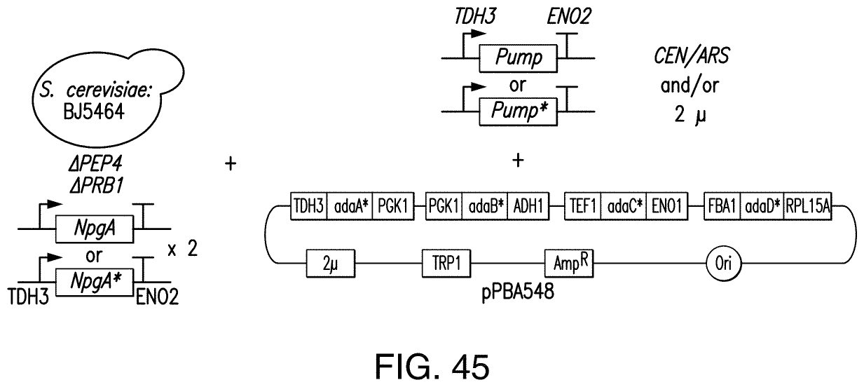

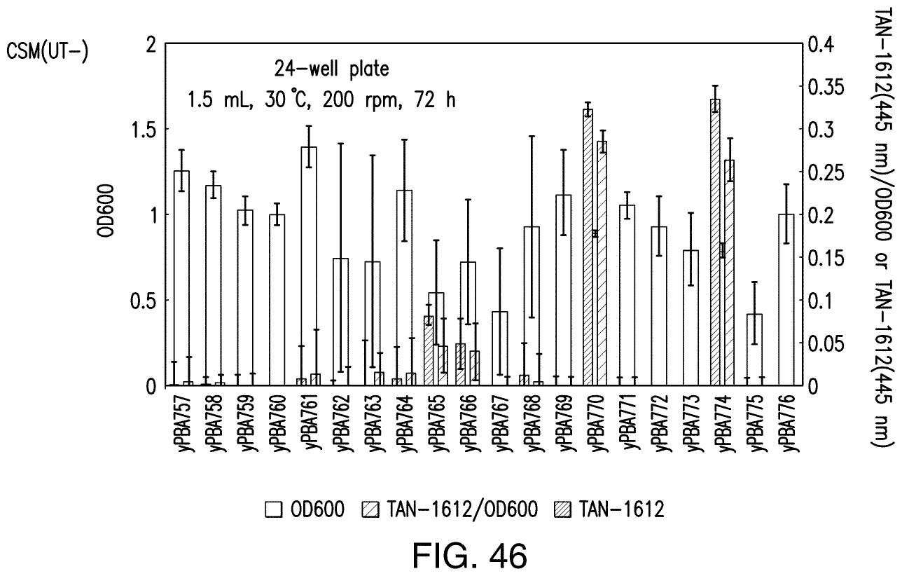

TAN-1612 production in S. cerevisiae. S. cerevisiae BJ5464 yeast strains were transformed with plasmids expressing efflux pumps as well as TAN-1612 biosynthetic pathway. (FIG. 45). BJ5464 cells were cultured in 24-2311 plate with 1 ml CSM at 30° C., shaken at 200 rpm for 72 hours. Cell growth of BJ5464 cultured in CSM in the presence of anhydrotetracycline (TAN-1612 analogue) and different efflux pumps were tested. (FIG. 46). OD600 by UV/Vis was measured.

For enzyme expression, promoter library and codon optimization were utilized. To address TAN-1612's toxicity issue, an efflux pump was used. (FIG. 47).

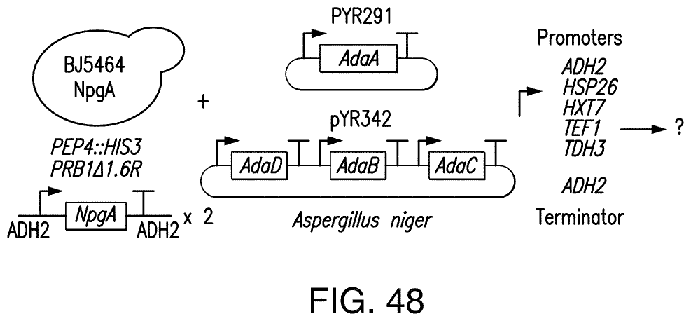

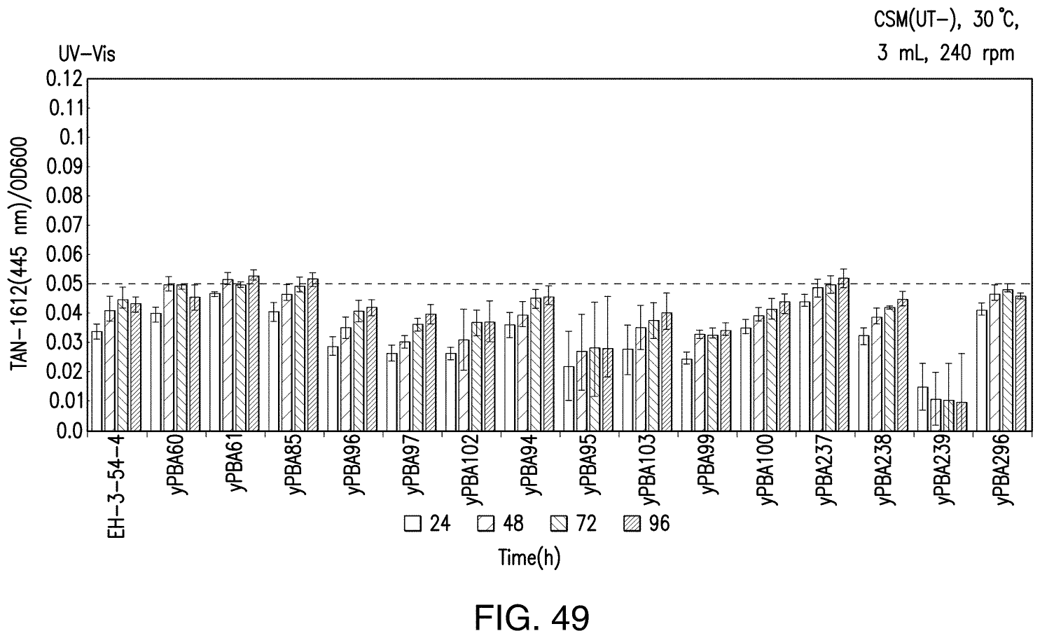

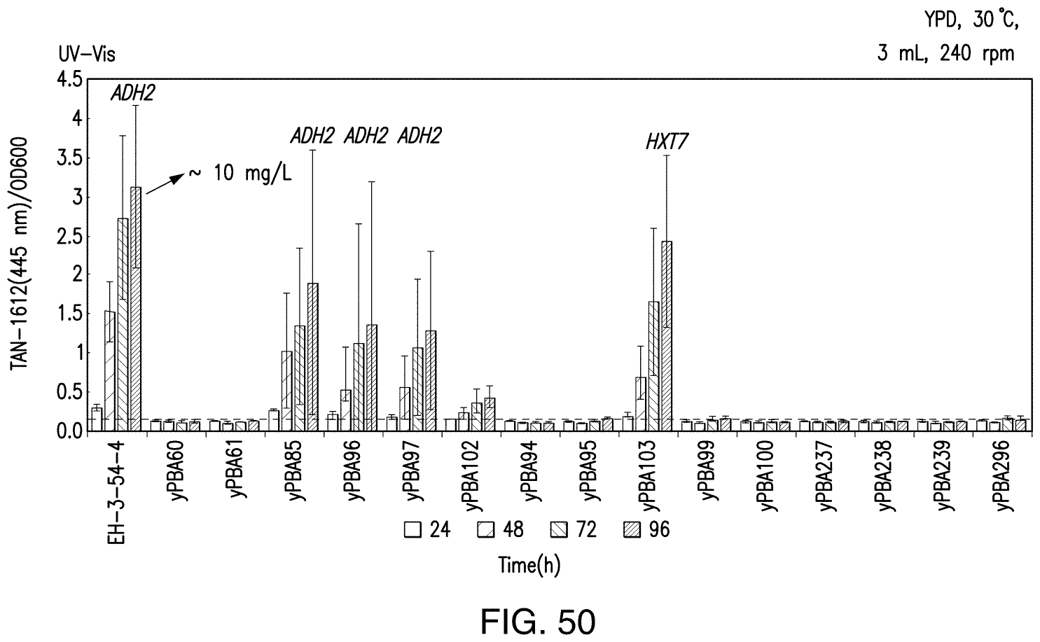

Identification of bottlenecks in TAN-1612 production in S. cerevisiae. Different promoters were tested for the expression of genes adaA, adaB, adaC and adaD using plasmids pYR291 and pYR342. (FIG. 48). TAN-1612 productivity in S. cerevisiae cultured in CSM (UT-) was tested. (FIG. 49). TAN-1612 productivity was reported as absorbance of TAN-1612 at 445 nm per cell growth (OD600). Tests were performed on 4 biological replicates grown in 3 mL of CSM(UT-) at 30 C, 240 rpm over 96 h. Dashed line (˜0.05) indicates background signal. TAN-1612 productivity in S. cerevisiae cultured in YPD was also tested. (FIG. 50). TAN-1612 productivity was reported as absorbance of TAN-1612 at 445 nm per cell growth (OD600). Tests were performed on 4 biological replicates grown in 3 mL of YPD at 30° C., 240 rpm over 96 h. Dashed line (˜0.05) indicates background signal. The previous tests indicated that gene NpgA was a potential bottleneck because of its expression is driven by promoter ADH2 whose activity is higher in YPD than CSM as previously shown by others.

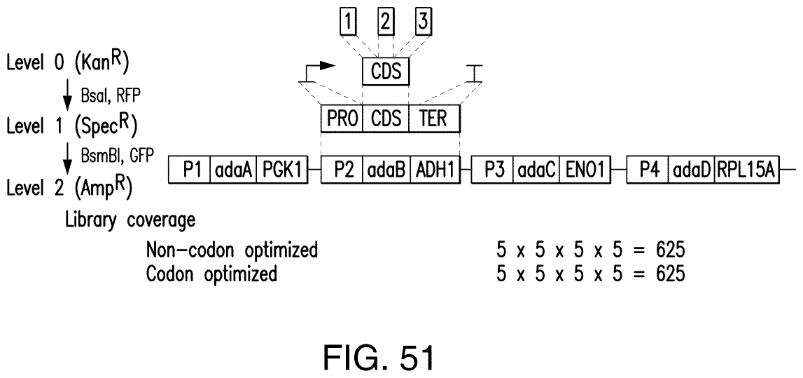

Building Promoter Library via Golden Gate Assembly. A Design Promoter Library was built via Golden Gate Assembly. Two different DNA sequences were used for each ada gene: non-codon optimized (from A. niger sequence) and codon optimized for yeast (S. cerevisiae) expression. (FIG. 51).

Characterization of Promoter Library. Library was characterized via Colony PCR (cPCR) and Sanger Sequencing. The non-codon optimized Library GGL contained 174 colonies and 46 of those were analyzed. The codon optimized Library GGL contained 175 colonies and 46 of those were analyzed. The two different promoter libraries were transformed and expressed in S. cerevisiae.

TABLE 23
UV/Vis screening summary.

| | | | | No biological replicates | |
|---|---|---|---|---|---|
| | | | | −96 h, 800 rpm, 30° C. | |
| UV/Vis screening summary | | | | CSM (T-), 400 μL | |
| | | | | N° colonies | |
| Background strain | Genotype | Library N* | GGL | screened | Hits |
| yPBA482 | BJ5464 ΔPEP4::pTDH3-Sb-NpgA-tENO2 ΔPRB1::pTDH3-Sb-NpgA-tENO2 | 1 | 174 | 256 | None |
| | | 2 | 175 | 256 | 1 |
| yPBA1277 | BJ5464 ΔPEP4::pADH2-Sb-NpgA-tENO2 ΔPRB1::pADH2-Sb-NpgA-tENO2 | 3 | 174 | 256 | 1 |
| | | 4 | 175 | 256 | 2 |
| yPBA1276 | BJ5464 ΔPEP4::pHSP26-Sb-NpgA-tENO2 ΔPRB1::pHSP26-Sb-NpgA-tENO2 | 5 | 174 | 256 | None |
| | | 6 | 175 | 256 | 2 |
| yPBA649 | BJ5464 ΔPEP4::pTDH3-Sb-NpgA-tENO2 ΔPRB1::pTDH3-Sb-NpgA-tENO2 + | 7 | 174 | 256 | 2 |
| | pPBA-paV116-pTDH3-Sb-ASPN:DRAFT_48051-yeast-cod_optimized- | 8 | 175 | 256 | 2 |
| | clean-tENO2 | | | | |
| yPBA1287 | BJ5464 ΔPEP4::pADH2-Sb-NpgA-tENO2 ΔPRB1::pADH2-Sb-NpgA-tENO2 + | 9 | 174 | 256 | 2 |
| | pPBA-paV116-pTDH3-Sb-ASPN:DRAFT_48051-yeast-cod_optimized- | 10 | 175 | 256 | None |
| | clean-tENO2 | | | | |
| yPBA1289 | BJ5464 ΔPEP4::pHSP26-Sb-NpgA-tENO2 ΔPRB1::pHSP26-Sb-NpgA-tENO2 + | 11 | 174 | 256 | 2 |
| | pPBA-paV116-pTDH3-Sb-ASPN:DRAFT_48051-yeast-cod_optimized- | 12 | 175 | 256 | 2 |
| | clean-tENO2 | | | | |
| yPBA478 | YMSO:6 ΔPEP4::pTDH3-Sb-NpGA-tENO2 ΔPRB1::pTDH3-Sb-NpgA- | 13 | 174 | 334 | 4 |
| | tENO2 | 14 | 175 | 384 | 3 |

For the non-codon optimized library, expression of genes adaA, adaB, adaC and adaD were analyzed under different promoters (Table 24). Three or four biological replicates were used. For the codon optimized library, expression of genes adaA, adaB, adaC and adaD were analyzed under different promoters (Table 25). Three or four biological replicates were used.

TABLE 24
UV/Vis screening summary for non-codon optimized library.
Summary of Library GGL - 174: Non-codon optimized

| Strain | Background | Colony | adaA | adaB | adaC | adaD |
|---|---|---|---|---|---|---|
| yPBA1350 | yPBA1275 | TAN-1612 Library-3-B well B2 | pPGK1-Sb | pCCW12-Sb | pCCW12-Sb | pTDH3-Sb |
| yPBA1351 | yPBA1287 | TAN-1612 Library-9-B well E4 | pCCW12-Sb | pCCW12-Sb | pTEF1-Sc | pTEF1-Sc |
| yPBA1352 | yPBA1289 | TAN-1612 Library-11-B well D9 | pTEF1-Sc | pPGK1-Sb | pTEF1-Sc | pTDH3-Sb |
| yPBA1353 | yPBA1289 | TAN-1612 Library-11-B well E10 | pTEF1-Sc | pHSP26-Sb | pTDH3-Sb | pPGK1-Sb |
| yPBA1399 | yPBA649 | TAN-1612 Library-7-A well E10 | pCCW12-Sb | pTEF1-Sc | pTDH3-Sb | pHSP26-Sb |
| yPBA1402 | yPBA1287 | TAN-1612 Library-9-B well H9 | pPGK1-Sb | pTDH3-Sb | pTEF1-Sc | pCCW12-Sb |
| yPBA1403 | yPBA478 | TAN-1612 Library-13-A2 well D2 | pPGK1-Sb | pHSP26-Sb | pTEF1-Sc | pHSP26-Sb |
| yPBA1404 | yPBA478 | TAN-1612 Library-13-B1 well F7 | pTEF1-Sc | pTDH3-Sb | pPGK1-Sb | pHSP26-Sb |
| yPBA1405 | yPBA478 | TAN-1612 Library-13-B2 well D6 | pPGK1-Sb | pHSP26-Sb | pCCW12-Sb | pHSP26-Sb |
| yPBA1406 | yPBA478 | TAN-1612 Library-13-B2 well G9 | pTEF1-Sc | pTEF1-Sc | pCCW12-Sb | pHSP26-Sb |

TABLE 25
UV/Vis screening summary for codon optimized library
Summary of Library GGL - 175: Codon optimized

| Strain | Background | Colony | adaA* | adaB* | adaC* | adaD* |
|---|---|---|---|---|---|---|
| yPBA1333 | yPBA482 | TAN-1612 Library-2-B well G9 | pHSP26-Sb | pPGK1-Sb | pTEF1-Sc | pTDH3-Sb |
| yPBA1334 | yPBA1275 | TAN-1612 Library-4-A well D2 | pHSP26-Sb | pHSP26-Sb | pHSP26-Sb | pHSP26-Sb |
| yPBA1335 | yPBA1275 | TAN-1612 Library-4-A well D7 | pHSP26-Sb | pHSP26-Sb | pHSP26-Sb | pHSP26-Sb |
| yPBA1336 | yPBA1277 | TAN-1612 Library-6-B well G7 | pHSP26-Sb | pPGK1-Sb | pTDH3-Sb | pHSP26-Sb |
| yPBA1337 | yPBA1277 | TAN-1612 Library-6-B well G9 | pHSP26-Sb | pHSP26-Sb | pTEF1-Sc | pTDH3-Sb |
| yPBA1354 | yPBA1289 | TAN-1612 Library-12-A well C2 | pHSP26-Sb | pPGK1-Sb | pTDH3-Sb | pHSP26-Sb |
| yPBA1355 | yPBA1289 | TAN-1612 Library-12-A well C3 | pHSP26-Sb | pHSP26-Sb | pTDH3-Sb | pHSP26-Sb |
| yPBA1400 | yPBA649 | TAN-1612 Library-8-B well C3 | pTDH3-Sb | pTDH3-Sb | pPGK1-Sb | pPGK1-Sb |
| yPBA1401 | yPBA649 | TAN-1612 Library-8-B well F2 | pHSP26-Sb | pHSP26-Sb | pTEF1-Sc | pHSP26-Sb |
| yPBA1407 | yPBA478 | TAN-1612 Library-14-B1 well F11 | pCCW12-Sb | pPGK1-Sb | pTEF1-Sc | pHSP26-Sb |
| yPBA1408 | yPBA478 | TAN-1612 Library-14-B1 well G9 | pHSP26-Sb | pPGK1-Sb | pTEF1-Sc | pHSP26-Sb |
| yPBA1409 | yPBA478 | TAN-1612 Library-14-B1 well G11 | pHSP26-Sb | pPGK1-Sb | pTEF1-Sc | pHSP26-Sb |

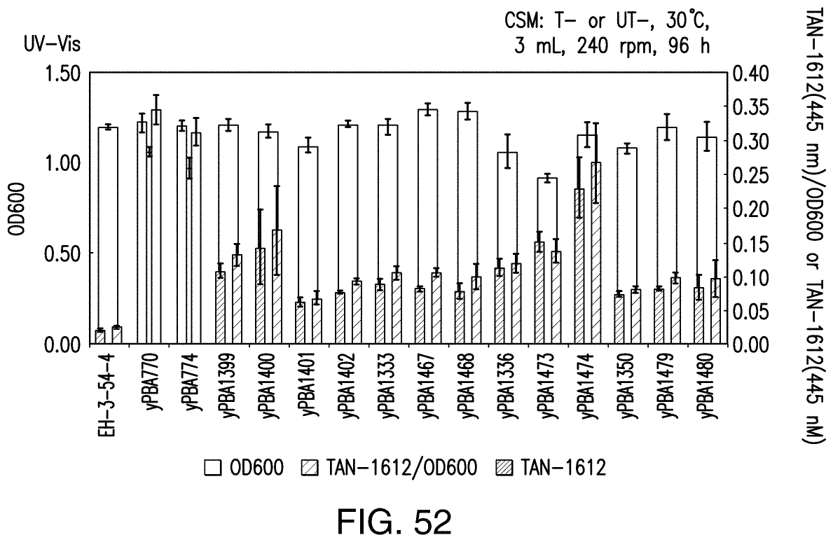

TAN-1612 productivity of the top TAN-1612 yeast strains in CSM (T-) Or CSM (UT) media. EH-3-54-4 is the original yeast strain. yPBA770 and yPBA774 were the two best producers. Each bar corresponds to 4 biological replicates. (FIG. 52). Yeast strains are depicted in Table 26.

TABLE 26
Genotypes of yPBA770, yPBA774 and yPBA1337.

| Strain | Genotype |
|---|---|
| yPBA770 | BJ5464 ΔPEP4::pTDH3-Sb-NpgA-tENO2 ΔPRBl::pTDH3-Sb-NpgA- |
| | tENO2 + pPBA-pAV116-pTDH3-Sb-ASPNIDRAFT_48051-yeast- |
| | cod_optimized-clean-tENO2 + pPBA-AV124_AmpR_Multigene-2μ-adaA-D_codon |
| yPBA774 | BJ5464 ΔPEP4::pTDH3-Sb-NpgA_codon_optimized-tENO2 |
| | ΔPRB1::pTDH3-Sb-NpgA_codon_optimized-tENO2 + pPBA-pAV116- |
| | pTDH3-Sb-ASPNIDRAFT 48051-yeast-cod optimized-clean-tENO2 + |
| | pPBA-AV124_AmpR_Multigene-2μ-adaA-D_codon |
| yPBA1337 | TAN-1612 producing strain in S. cerevisiae without efflux pump |
| | BJ5464 ΔPEP4::pHSP26-Sb-NpgA-tENO2 ΔPRB1::pHSP26-Sb-NpgA- |
| | tENO2 + pPBA-AV124_AmpR_Multigene-2μ-pHSP26-adaA_yeast-codon- |
| | opt-tPGK1_pHSP26-adaB_yeast-codon-opt-tADH1_pTEF1-adaC_yeast- |
| | codon-op t-tENO1_pTDH3-adaD_yeast-codon-opt-tRPL15A |

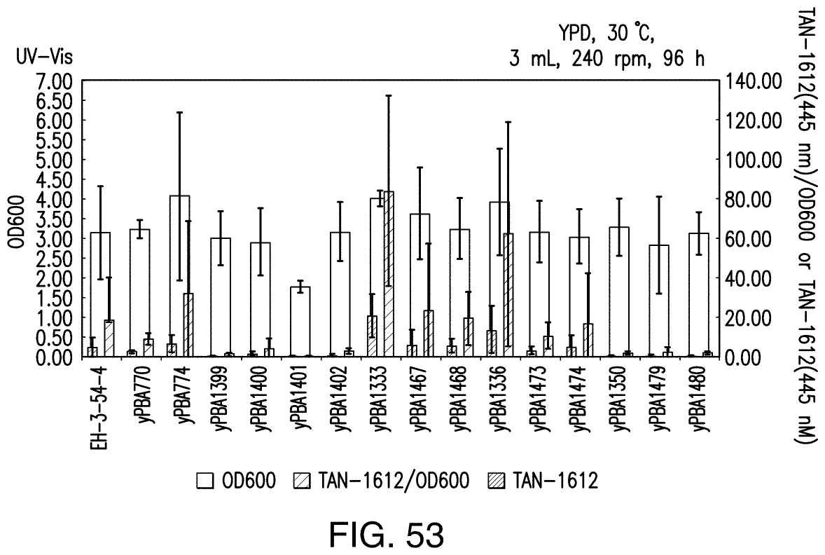

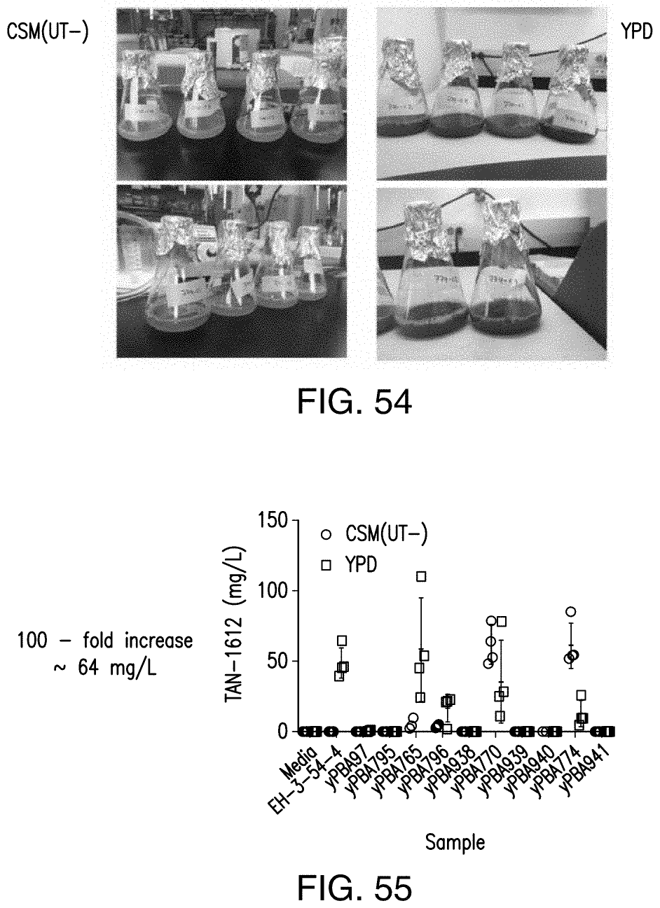

TAN-1612 productivity of the top TAN-1612 yeast strains in YPD Medium. EH-3-54-4 is the original yeast strain. yPBA770 and yPBA774 are the two best producers. Each bar corresponds to 4 biological replicates. (FIG. 53). TAN-1612 flask production both in CSM (UT-) and YPD media. (FIG. 54).

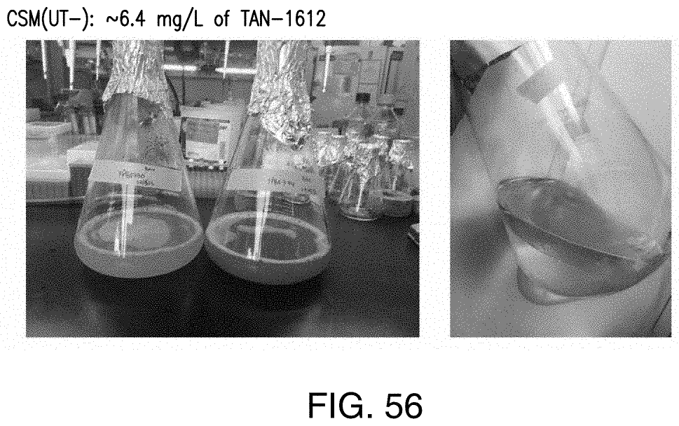

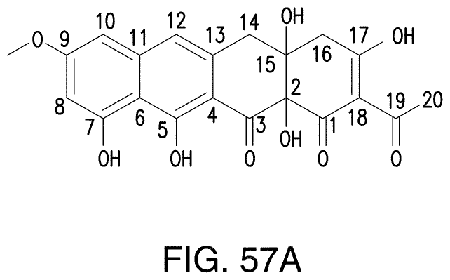

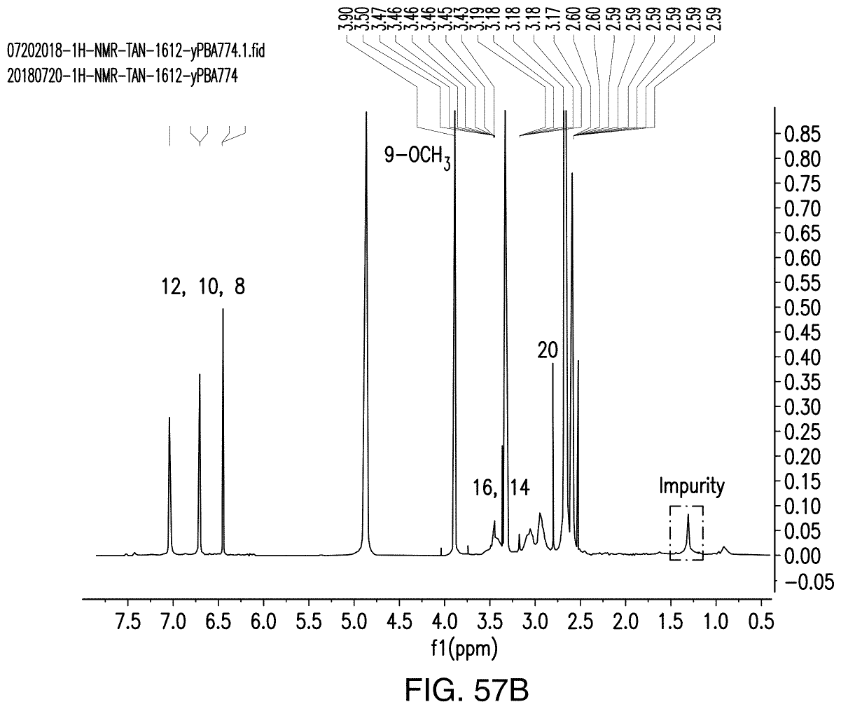

Quantification of TAN-1612 titers by Supercritical FLIUD Chromatography Mass Spectrometry (SFC-MS). yPBA770 and yPBA774 are the two best TAN-1612 yeast producer strains in CSM (UT-) representing a 100-fold increase with respect to EH-3-54-4 strain: about 64 mg/L. (FIG. 55, FIG. 56). Purified TAN-1612 in S. cerevisiae was characterized by NMR spectrum. (FIGS. 57A and 57B).

TABLE 27
Isolation of TAN-1612 in A. niger versus S. cerevisiae.

| | A. niger | S. cerevisiae | |
|---|---|---|---|
| TAN-1612 pump? | Yes | Yes | No |
| | (ATCC 1015 + pYR311) | (yPBA774) | (BJ5464-NpgA) |
| Isolation time | 16-25 days | ~13-16 days | — |
| Titers reported (mg/L) | ~400 | Not reported | CSM(UT-): - |
| by Tanq lab (2011) | | | YPD: ~10 |
| Titers isolated (mg/L) by | ~60-100 | CSM(UT-): ~6.4 | Not determined |
| Cornish lab | | | |
| Titers reported (mg/L) by | Not determined | CSM(UT-): ~61 | CSM(UT-): ~0.62 |
| Cornish lab | | YPD: ~12 | YPD: ~49 |

### Example 7. TAN-1612: Metabolic Engineering Approaches to Increase Biosynthetic Titers in Yeast

This example employs the FP and Y3H technologies for the metabolic engineering (ME) of yeast strains for high titer production of novel tetracycline analogs for therapeutic discovery. Fermentation is the method of choice for tetracycline production—all nine FDA-approved tetracyclines are produced by fermentation of either the final product (“biosynthesis”) or an intermediate that is subsequently chemically derivatized (“semisynthesis”). However, with bacterial resistance against all nine of FDA-approved tetracycline analogs, there is a need for new strategies to discover novel tetracyclines. Synthetic chemistry developed by Myers and co-workers has opened up access to modifications at the D-ring. Here, yeast is used to enable modifications at the 6α-, 4a- and 4α-positions using biosynthesis and semisynthesis based on the fungal tetracycline TAN-1612—an unexplored scaffold with unique chemical handles. The fungal origin of TAN-1612 makes S. cerevisiae an ideal production host.

A S. cerevisiae strain with good yield of the tetracycline TAN-1612 in synthetic media by optimizing the expression of the TAN-1612 pathway as well as the accessory protein NpgA and expressing a supposed efflux pump to mitigate TAN-1612 toxicity was engineered. Optimization was achieved using the v1.0 TAN-1612 FP and the v1.0 Y3H assays described, respectively and confirmed by MS (FIG. 59). The yields of this v2.0 TAN-1612 producer strain in synthetic media help tackle the analogue production outlined in Example 4.

This v2.0 TAN-1612 producer strain to generate a high-titer, modular tetracycline analogue production platform is built. The FP and the Y3H assay are employed to search large combinatorial metabolically engineered libraries of yeast strains to increase the production titers (>100 mg/L in YPD and synthetic media). The biosynthesized TAN-1612 scaffold into tetracycline analogs are diversified by tailoring enzymes that can convert TAN-1612 into both novel tetracycline final products and intermediates for further semi-synthesis.

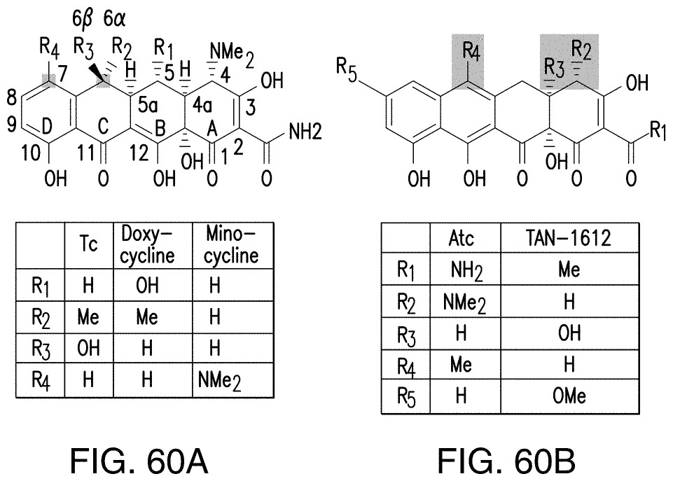

High-titer biosynthesis of the scaffold TAN-1612 is pivotal for its enzymatic and chemical derivatization into a library of analogs. The FP and Y3H assays combined with rounds of sexual crossing of libraries are used to search for multi-parameter optimized metabolic solutions to high-titer production. In primary libraries (PLs) a set of rational and randomized pathway and strain background diversification strategies are combined in order to (i) increase the available pool of pathway precursors, (ii) to enhance pathway flux and (iii) to mediate TAN-1612 growth inhibition. PLs (˜10⁴ variants each) can be screened immediately for increased titer using the available TAN-1612 FP assay (FIGS. 60A and 60B). Eventually, full and prescreened PLs are combined to secondary libraries (SLs, ˜10⁸-10¹⁰ variants) by yeast mating and searched by the Y3H assay for metabolic solutions that simultaneously optimize all three parameters.

Reconstituting the TAN-1612 pathway in S. cerevisiae requires co-expression of four biosynthetic enzymes derived from the fungus A. niger, and one helper enzyme from the fungus Aspergillus nidulans. Both precursors of the TAN-1612 pathway-acetyl CoA and malonyl CoA—can be pulled from S. cereverisiae's carbohydrate and lipid metabolism. All required cofactors—flavin adenine dinucleotide (FAD), S-Adenosyl methionine (SAM) and coenzyme A—are native to S. cerevisiae. The four biosynthetic enzymes are: AdaA, a nonreducing polyketide synthase (NRPKS); AdaB, a metallo-β-lactamase-type thioesterase; AdaC, a FAD-dependent monooxygenase (FMO); and AdaD, a SAM-dependent O-methyltransferase. The helper enzyme is NpgA, a 4′-phosphopantetheinyl transferase that adds the essential 4′-phosphopantetheine prosthetic group from coenzyme A onto the acyl carrier unit of the NRPKS AdaA (FIG. 58).

The primary library (PL1) focuses on increasing the pool of the two TAN-1612 precursors acetyl-CoA and malonyl-CoA. TAN-1612 is detected as essentially the only polyketide product of the v1.0 TAN-1612 producer, indicating that a likely metabolic bottleneck in TAN-1612 biosynthesis is at or before AdaA—the first enzyme condensing the precursors into the polyketide ring structure. Enhancing metabolic flux towards AdaA includes the combinatorial gene titration of ALD6 (acetaldehyde dehydrogenase), ADH2 (alcohol dehydrogenase), as well as ACC1 (acetyl-CoA carboxylase) using promoters of varying strengths as indicated below for PL2 (FIG. 58). The malonyl CoA levels are further increased by reducing negative regulation, as well as expression of a Salmonella enterica acetylation-insensitive acetyl-CoA synthetase (acsSE). Heritable recombination (HR) is used for in-vivo multi-locus targeted mutagenesis directly in the yeast genome. This PL1 of ˜10³ variants was screened for increased TAN-1612 production with the immediately available FP assay (FIG. 59) and the results of top producers are verified by LCMS.

The second primary library (PL2) can enhance flux through the TAN-1612 pathway. A promoter library (10 promoters) for each of the four genes of the TAN-1612 pathway and NpgA to find the right combination of gene titration that optimizes flux was built. This pathway library was built in vitro by Yeast Golden Gate. To optimize for precursor conversion and pathway flux simultaneously PL2 (˜10⁴ variants) was crossed with the best performers of PL1 (˜best 10%, 10² variants) by mating and assay this resulting secondary library (SL1, ˜106 variants) for increased TAN-1612 production using Y3H FACS. The promoter library featured promoters of different strengths and cell growth phase activity profiles (TEF 1, PGK1, PYK1, HXT7, ADH1, CYC1, ADH2, PCK1, MLS1 and ICL1). Specifically, the late-stage promoters included in the promoter library mitigated toxicity of pathway products during exponential growth.

The third primary library (PL3) mediates TAN-1612 high-titer toxicity by merging three complimentary routes. This example (i) optimizes performance of an efflux pump in order to reduce intracellular TAN-1612 concentrations, (ii) screens a large number of wild yeast isolates for higher TAN-1612 tolerance and use them for background crossing and (iii) evolves the v2.0 producer for TAN-1612 resistance through random mutagenesis and selection. In preliminary results, TAN-1612 and anhydrotetracycline (Atc) growth inhibition in concentrations of 10 mg/L was confirmed. In order to increase TAN-1612 tolerance the v2.0 producer co-expresses the supposed efflux pump ASPNIDRAFT_48051 (GenBank: EHA19824.1) that was obtained by genome-mining from the natural producer strain A. niger. This heterologous expression showed an increase of TAN-1612 production as verified by the FP assay and by MS (FIGS. 60A and 60B). This can improve pump performance via gene titration and genome-mines other efflux pumps. PL3 further includes of a large number (>50) of wild S. cerevisiae isolates that are screened for higher tolerance to TAN-1612 using a growth-based microtiter plate assay.

Wild S. cerevisiae isolates show diverse phenotypes while keeping the ability to mate with laboratory S. cerevisiae strains. Hence, this natural genomic diversity can be harnessed by breeding it into laboratory strains. Simultaneously, the v2.0 producer itself can be evolved towards higher TAN-1612 tolerance using UV-treatment and transposon-mutagenesis followed by selection in a chemostat with Atc, the commercially available proxy for TAN-1612 (FIG. 61). PL3 members that tolerate the highest TAN-1612 levels are crossed sequentially with PL1, PL2 and SL1 to yield a large secondary library (SL2, ˜10⁹) that can be searched for the highest-titer production using the Y3H FACS screen. If the above does not increase TAN-1612 titers, titers of the key intermediate 3 can be improved instead of TAN-1612 as procedures 1 and 2 can decrease C-ring planarity, resulting in a reduction in toxicity to eukaryotes (FIG. 33A-33C).

### Example 8. Adding A-Ring Functional Groups Required for Antibiotic Activity to TAN-1612

This Example illustrates strategies for adding functional groups at the 2- and 4-positions of TAN-1612, creating a promising anhydrotetracycline scaffold for tetracycline antibiotics. (Step 1, FIG. 61). Both the 2-carboxamido and the 4α-dimethylamino groups are necessary for antibiotic activity of tetracyclines.

Genes to produce the malonamoyl CoA precursor and evolve the Ada enzymes to accept malonamoyl CoA as a substrate towards 2-deacetyl-2-carboxamindo TAN-1612 are encoded. 2-carboxamido group is incorporated in TAN-1612 derivatives by encoding OxyD and OxyP from the oxytetracycline pathway of S. rimosus in the v2.0 TAN-1612 producer. Alternatively, homologous enzymes to the Ada enzymes from the anhydrotetracycline analogue viridicatumtoxin pathway of Penicillium aethiopicum are incorporated. Primary libraries are built towards a yeast strain producing malonamoyl CoA (i) promoter libraries for each of the heterologous genes (10²-10³) (ii) >20 genome mined enzymes for malonamate biosynthesis, the precursor to malonamoyl CoA(125) and (iii) mutants of AdaA in the substrate binding site for recognizing the malonamoyl CoA starting material in the v2.0 TAN-1612 producer (>3.2×10⁶). The primary libraries by HR are then crossed and the 10⁸ secondary library for TAN-1612 production using the Y3H assay is selected.

4α-dimethylamino functionality is accessed by employing an oxygenase oxidase, transaminase and methylase from the oxytetracycline pathway of S. rimosus by generating three libraries: (i) >50 yeast isolates for oxygenase and oxidase function on TAN 1612, (ii) genome mine >20 oxygenase and oxidase enzymes from higher eukaryotes, and (iii) mutants of OxyE and OxyL, OxyQ and OxyT for TAN-1612 specificity (>3.2*10⁶ each). The primary libraries are screened sequentially by using the Y3H FACS with a TetR mutant binding to intermediate compounds (compounds 12-14 of FIG. 62) and once hits are found, the primary libraries of both next and previous steps by HR and screen (size >10⁹) with the Y3H/FP assay are crossed. The 4α-dimethylamino can also be installed after introducing the desired functionality at the 6-position.

The synthesis process is illustrated in FIG. 62.

### Example 9. Adding A-Ring Functional Groups Required for Antibiotic Activity to TAN-1612

6α-hydroxylation is a required handle that can be glycosylated to generate a library of 6-glycosides. DacO1, PgaE, and SsfO1 the 6α-hydroxylase homologs of the 6β-hydroxylase FAD-dependent monooxygenase OxyS and genome-mine other hydroxylases (e.g. CtcN) are employed. After testing these enzymes' activity towards their native substrate they are evolved for TAN-1612 6α-hydroxylation by constructing a library of 5 fully randomized amino acids within 5 Å to the modeled substrate, as predicted based on the crystal structure of the homologous Aklavinone-11-Hydroxylase that was crystalized with its native substrate aklavinone. The resulting libraries (3.2×10⁶) are screened by the Y3H FACS with TetR screened to bind compound 15 of FIG. 62.

Secondly, the 5a(11a)-enone reduction is crucial for the required stereochemical configuration for antibiotic activity. DacO4, a bacterial F420-dependent reductase, that performs the analogous 5a(11a) reduction in the dactylocycline pathway, as well as DacO4 homologs are employed. Members of the highly promiscuous Old Yellow Enzyme family, that catalyze enone reduction to ketones in a variety of substrates is also screened. These enzymes do not require F420, a cofactor non-natural to yeast, simplifying their functional heterologous expression. To biosynthesize Fo, a functional alternative to cofactor F4₂₀, v2.0 TAN-1612 producer an Fo synthase from Chlamydomonas reinhardtii is co-expressed, or alternatively, the strains are provided with synthetic Fo.

Lastly, the glycodiversification of the 6α-hydroxy handle generated by Steps 5 and 6 of (FIG. 61) with a library of activated glycosides is crucial for the activity against tetracycline-resistant strains. The huge diversity of glycosides and the natural substrate promiscuity of glycosyltransferases enables targeting gram-negative resistant infections. Glycosyltransferase DacS8, from the dactylocycline biosynthetic pathway, and a diverse set of glycoside biosynthesis enzymes is employed.

### Example 10. Genetic Modification of Yeast to Express Toxin Peptides

An S. cerevisiae parental strain was transformed with two different natural S. cerevisiae peptide killer toxins, K1, K2 and K28, to generate genetically modified S. cerevisiae strains secreting K1, K2 or K28 killer toxins. The amino acid and nucleotide sequences of the K1, K2 or K28 killer toxins are shown in Table 28. The three killer toxins encode for their own signal sequences which are then processed by the KEX2 and/or KEX1 proteases at putative processing sites (KR in the amino acid sequence is a higher affinity site than ER) to form a mature alpha/beta heterodimer.

Halo assays were performed to monitor the inhibition of growth of a potentially susceptible strain. Specifically, a potentially susceptible strain, e.g., S. boulardii, was inoculated on a plate in a uniform layer of soft agar (lawn) and the genetically modified S. cerevisiae strain expressing the killer toxin was inoculated in the middle of the plate as a concentrated liquid culture. Halo assays with myceliated fungus Ganoderma resinaceum was performed by inoculating the genetically modified S. cerevisiae strain expressing the killer toxin as a lawn and Ganoderma resinaceum was inoculated in the middle as an agar chunk. G. resinaceum growth was monitored not as a cell density, but as a development of a mycelium webbing.

The agar plates were imaged in ChemiDoc at Pro-Q Emerald 300 setting to visualize cell density. Higher cell densities appear brighter, while low cell densities appear darker. Assays were done in technical triplicate and images were taken after 48-h incubation.

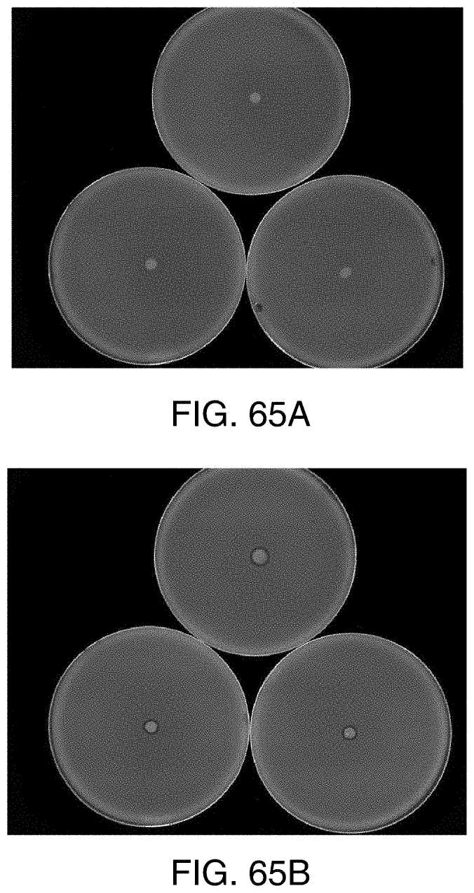

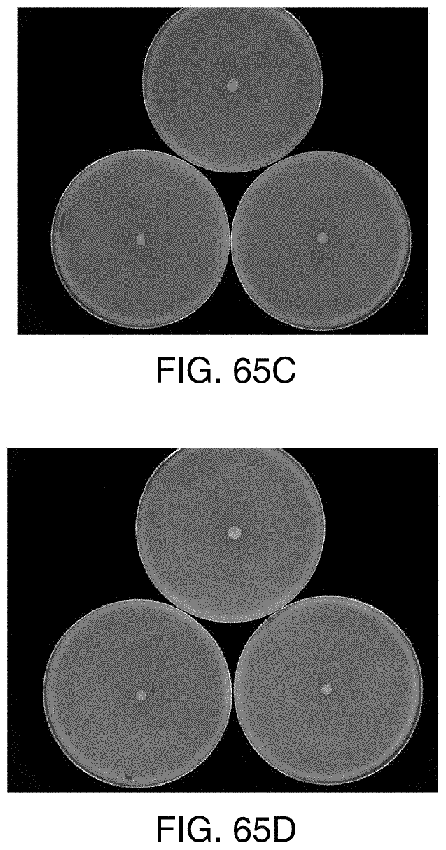

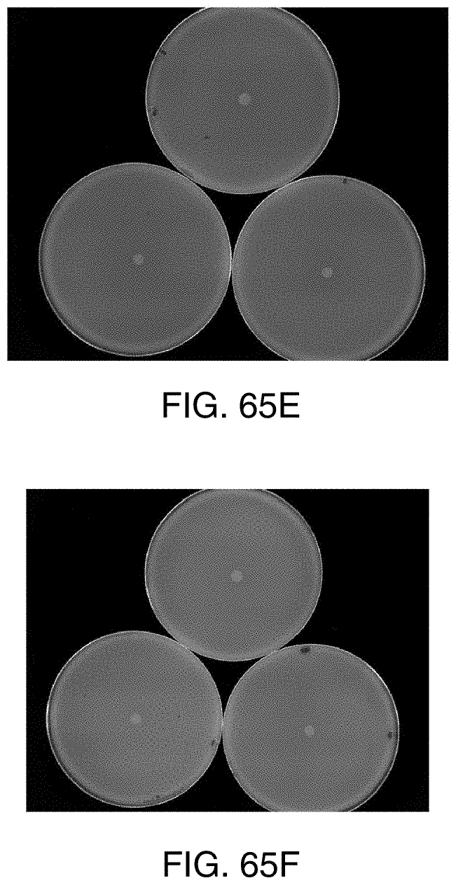

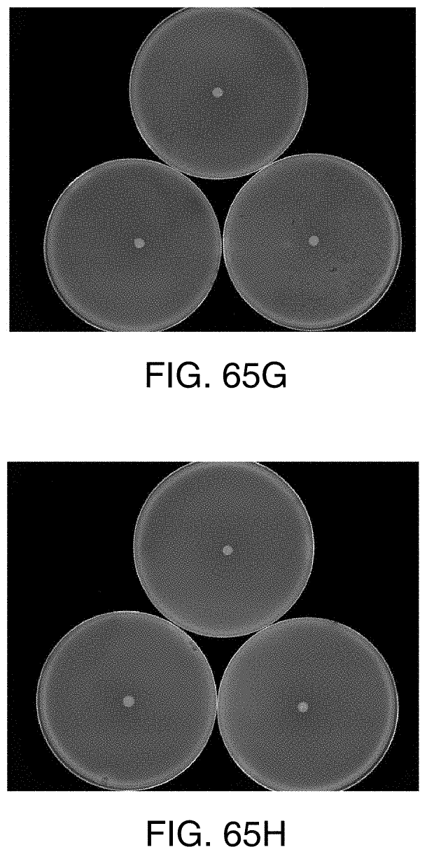

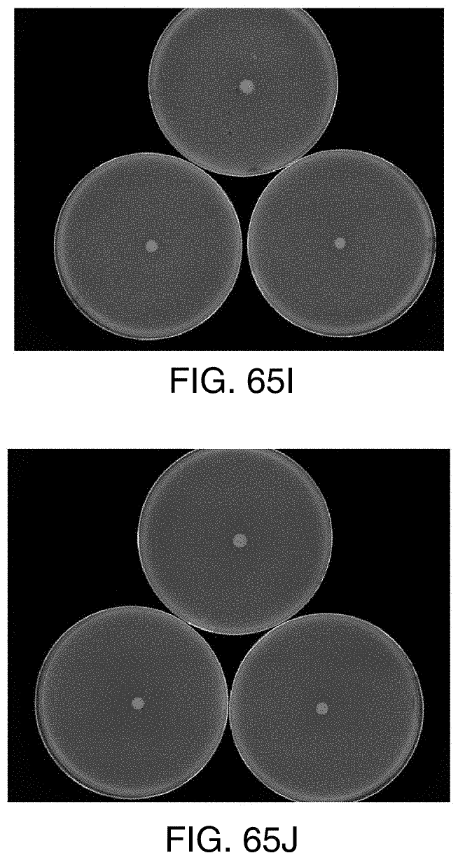

As shown in FIG. 65B, S. cerevisiae secreting the killer toxin K2 led to immediate growth inhibition of S. boulardii as evidenced by the appearance of dark low cell density halo around secreting strain dropped in the middle. No other killer toxin tested showed growth inhibition to any other tested strain indicating strain-specific activity of the tested toxins (FIGS. 65A and 65C-J).

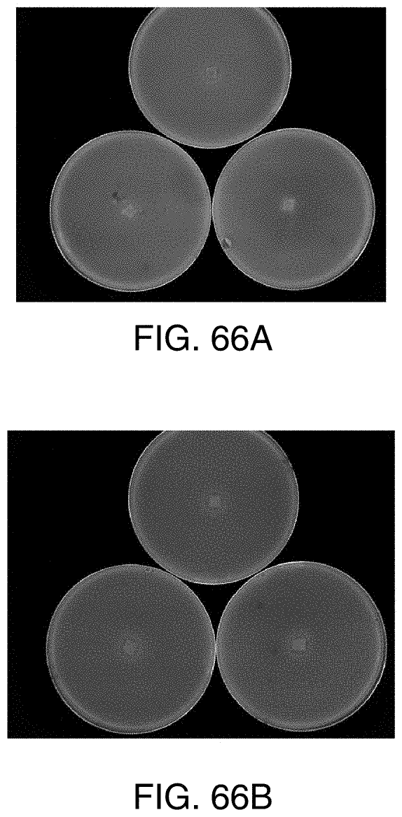

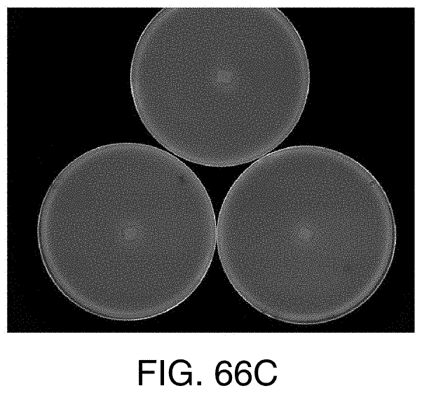

As shown in FIGS. 66A-66C, S. cerevisiae expressing killer toxin K2, or K28, or no toxin was inoculated on a plate in a uniform layer of soft agar (lawn) and an agar chunk with G. resinaceum was inoculated in the middle of the plate. The plates were incubated for 48 h and they were imaged in ChemiDoc at Pro-Q Emerald 300 setting to visualize mycelium webbing. The webbing appears as a lighter halo expanding from the agar chunk in the middle of the plate. No significant webbing inhibition was observed. Further experiments were performed to show the effect temperature has on the activity of K2. As shown in FIG. 64, K2-secreting yeast cells were more effective at killing S. cerevisiae cells at 24° C. than at 30° C.

TABLE 28
Sequences of killer toxin peptides.

Signal peptide sequence is italicized, alpha subunit is underlined, beta subunit in
uncapitalized and protease cleavage sites are bolded.

| | |
|---|---|
| K1 | Amino acid Sequence |
| | MTKPTQVLVRSVSILFFITLLHLVVALNDVAGPAETAPVSLLPREAPWYDKIWEVKDWLLQRATDGNWGKSITWGSFVASDAGVVIF |
| | GINVCKNCVGERKDDISTDCGKQTLALLVSIFVAVTSGHHLIWGGNRPVSQSDPNGATVARRDISTVADGDIPLDFSALNDILNEHG |
| | ISILPANASQYVKRSDTAEHTTSFVVTNNYTSLHTDLIHHGNGTYTTFTTPHIPAVAKRYVYPMCEHGIKASYCMALNDAMVSANGN |
| | LYGLAEKLFSEDEGQWETNYYKLYWSTGQWIMSMKFIEESIDNANNDFEGCDTGH (SEQ ID NO: 170). |
| | Italicized text (SEQ ID NO: 171). Nonitalicized capitalized text (SEQ ID NO: 172). |
| | Nucleotide Sequence |
| | ATGACCAAACCGACCCAGGTGCTGGTGCGCAGCGTGAGCATTCTGTTTTTTATTACCCTGCTGCATCTGGTGGTGGCGCTGAACGAT |
| | GTGGCGGGCCCGGCGGAAACCGCGCCGGTGAGCCTGCTGCCGCGCGAAGCGCCGTGGTATGATAAAATTTGGGAAGTGAAAGATTGG |
| | CTGCTGCAGCGCGCGACCGATGGCAACTGGGGCAAAAGCATTACCTGGGGCAGCTTTGTGGCGAGCGATGCGGGCGTGGTGATTTTT |
| | GGCATTAACGTGTGCAAAAACTGCGTGGGCGAACGCAAAGATGATATTAGCACCGATTGCGGCAAACAGACCCTGGCGCTGCTGGTG |
| | AGCATTTTTGTGGCGGTGACCAGCGGCCATCATCTGATTTGGGGCGGCAACCGCCCGGTGAGCCAGAGCGATCCGAACGGCGCGACC |
| | GTGGCGCGCCGCGATATTAGCACCGTGGCGGATGGCGATATTCCGCTGGATTTTAGCGCGCTGAACGATATTCTGAACGAACATGGC |
| | ATTAGCATTCTGCCGGCGAACGCGAGCCAGTATGTGAAACGCAGCGATACCGCGGAACATACCACCAGCTTTGTGGTGACCAACAAC |
| | TATACCAGCCTGCATACCGATCTGATTCATCATGGCAACGGCACCTATACCACCTTTACCACCCCGCATATTCCGGCGGTGGCGAAA |
| | CGCTATGTGTATCCGATGTGCGAACATGGCATTAAAGCGAGCTATTGCATGGCGCTGAACGATGCGATGGTGAGCGCGAACGGCAAC |
| | CTGTATGGCCTGGCGGAAAAACTGTTTAGCGAAGATGAAGGCCAGTGGGAAACCAACTATTATAAACTGTATTGGAGCACCGGCCAG |
| | TGGATTATGAGCATGAAATTTATTGAAGAAAGCATTGATAACGCGAACAACGATTTTGAAGGCTGCGATACCGGCCATTAG |
| | (SEQ ID NO: 173). Italicized text (SEQ ID NO: 174). Nonitalicized capitalized text |
| | (SEQ ID NO: 175). |
| K2 | Amino acid Sequence |
| | MKETTTSLMQDELTLGEPATQARMCVRLLRFFIGLTITAFIIAACIIKSATGGSGYSKAVAVRGEADTPSTIVGOLVERGGFQAWAV |
| | GAGIYLFAKIAYDTSKVTAAVCNPEALIAITSYVAYAPTLCAGAYVIGAMSGAMSAGLALYAGYKGWGWGGPGGMAEREDVASFYSP |
| | LLNNTLYVGGDHTADYDSELATILGSVYNDVVHLGVYYDNSTGIVKRDSRPSMISWTVLHDNMMITSYHRPDQLGAAATAYKAYTTN |
| | TTRVGKRqdgewvsysvygenvdyerypvahlqeeadacyeslgnmitsqvqpctqrecyamdqkvcaavgfssdagvnsamvgeay |
| | fyayggvdgecdsg (SEQ ID NO: 176). Italicized text (SEQ ID NO: 177). |
| | Underlined capitalized text (SEQ ID NO: 178). Noncapitalized text (SEQ ID NO: 179). |
| | Nucleotide Sequence |
| | ATGAAAGAAACCACCACCAGCCTGATGCAGGATGAACTGACCCTGGGCGAACCGGCGACCCAGGCGCGCATGTGCGTGCGCCTGCTG |
| | CGCTTTTTTATTGGCCTGACCATTACCGCGTTTATTATTGCGGCGTGCATTATTAAAAGCGCGACCGGCGGCAGCGGCTATAGCAAA |
| | GCGGTGGCGGTGCGCGGCGAAGCGGATACCCCGAGCACCATTGTGGGCCAGCTGGTGGAACGCGGCGGCTTTCAGGCGTGGGCGGTG |
| | GGCGCGGGCATTTATCTGTTTGCGAAAATTGCGTATGATACCAGCAAAGTGACCGCGGCGGTGTGCAACCCGGAAGCGCTGATTGCG |
| | ATTACCAGCTATGTGGCGTATGCGCCGACCCTGTGCGCGGGCGCGTATGTGATTGGCGCGATGAGCGGCGCGATGAGCGCGGGCCTG |
| | GCGCTGTATGCGGGCTATAAAGGCTGGCAGTGGGGCGGCCCGGGCGGCATGGCGGAACGCGAAGATGTGGCGAGCTTTTATAGCCCG |
| | CTGCTGAACAACACCCTGTATGTGGGCGGCGATCATACCGCGGATTATGATAGCGAACTGGCGACCATTCTGGGCAGCGTGTATAAC |
| | GATGTGGTGCATCTGGGCGTGTATTATGATAACAGCACCGGCATTGTGAAACGCGATAGCCGCCCGAGCATGATTAGCTGGACCGTG |
| | CTGCATGATAACATGATGATTACCAGCTATCATCGCCCGGATCAGCTGGGCGCGGCGGCGACCGCGTATAAAGCGTATACCACCAAC |
| | ACCACCCGCGTGGGCAAACGCcaggatggcgaatgggtgagctatagcgtgtatggcgaaaacgtggattatgaacgctatccggtg |
| | gcgcatctgcaggaagaagcggatgcgtgctatgaaagcctgggcaacatgattaccagccaggtgcagccgtgcacccagcgcgaa |
| | tgctatgcgatggatcagaaagtgtgcgcggcggtgggctttagcagcgatgcgggcgtgaacagcgcgatggtgggcgaagcgtat |
| | ttttatgcgtatggcggcgtggatggcgaatgcgatagcggctaa (SEQ ID NO: 180). Italicized text |
| | (SEQ ID NO: 181). Underlined capitalized text (SEQ ID NO: 182). Noncapitalized |
| | text (SEQ ID NO: 183). |
| K28 | Amino acid Sequence |
| | MESVSSLFNIFSTIMVNYKSLVLALLSVSNLKYARGMPTSERQQGLEERDFSAATCVLMGAEVGSWGMVYSGQKVESWILYVLTGIT |
| | TMSAIVDEIDYYASHMPLSVVGENSGLQIVRDTIVTLVMAGLTASANKVISKTENAENIQSRSLIPGLLSMDYNSTHTMAINLEDVF |
| | SELGWDIDTSDSSGLYKRDDNSVTLHLGDVPALGTSNTIIPNAVMQIYNNASFAFGFAPHSNGNSTGLQKRASIDDAVWLQSAYGIA |
| | YSAWIGSENVGSYDEHLAEANGMANYWTSECSKYNGVIWGDESDACGNWLASQRLDIVSHSTGNYYRDVNLCGDDEARCHDELR |
| | (SEQ ID NO: 184). Italicized text (SEQ ID NO: 185). Underlined capitalized text |
| | (SEQ ID NO: 186). Nonunderlined capitalized text (SEQ ID NO: 187). |
| | Nucleotide Sequence |
| | ATGGAAAGCGTGAGCAGCCTGTTTAACATTTTTAGCACCATTATGGTGAACTATAAAAGCCTGGTGCTGGCGCTGCTGAGCGTGAGC |
| | AACCTGAAATATGCGCGCGGCATGCCGACCAGCGAACGCCAGCAGGGCCTGGAAGAACGCGATTTTAGCGCGGCGACCTGCGTGCTG |
| | ATGGGCGCGGAAGTGGGCAGCTGGGGCATGGTGTATAGCGGCCAGAAAGTGGAAAGCTGGATTCTGTATGTGCTGACCGGCATTACC |
| | ACCATGAGCGCGATTGTGGATGAAATTGATTATTATGCGAGCCATATGCCGCTGAGCGTGGTGGGCGAAAACAGCGGCCTGCAGATT |
| | GTGCGCGATACCATTGTGACCCTGGTGATGGCGGGCCTGACCGCGAGCGCGAACAAAGTGATTAGCAAAACCGAAAACGCGGAAAAC |
| | ATTCAGAGCCGCAGCCTGATTCCGGGCCTGCTGAGCATGGATTATAACAGCACCCATACCATGGCGATTAACCTGGAAGATGTGTTT |
| | AGCGAACTGGGCTGGGATATTGATACCAGCGATAGCAGCGGCCTGTATAAACGCGATGATAACAGCGTGACCCTGCATCTGGGCGAT |
| | GTGCCGGCGCTGGGCACCAGCAACACCATTATTCCGAACGCGGTGATGCAGATTTATAACAACGCGAGCTTTGCGTTTGGCTTTGCG |
| | CCGCATAGCAACGGCAACAGCACCGGCCTGCAGAAACGCGCGAGCATTGATGATGCGGTGTGGCTGCAGAGCGCGTATGGCATTGCG |
| | TATAGCGCGTGGATTGGCAGCGAAAACGTGGGCAGCTATGATGAACATCTGGCGGAAGCGAACGGCATGGCGAACTATTGGACCAGC |
| | GAATGCAGCAAATATAACGGCGTGATTTGGGGCGATGAAAGCGATGCGTGCGGCAACTGGCTGGCGAGCCAGCGCCTGGATATTGTG |
| | AGCCATAGCACCGGCAACTATTATCGCGATGTGAACCTGTGCGGCGATGATGAAGCGCGCTGCCATGATGAACTGCGCTAA |
| | (SEQ ID NO: 188). Italicized text (SEQ ID NO: 189). Underlined capitalized text |
| | (SEQ ID NO: 190). Nonunderlined capitalized text (SEQ ID NO: 191). |

### Example 11. Treatment of Inflammation by Genetically-Engineered Cells Producing TAN-1612

This Example shows the effect TAN-1612-producing yeast have on inflammation in lipopolysaccharide (LPS)-stimulated mammalian cells. LPS stimulates cells and initiates the inflammatory process.

On Day 0, yeast cells are pre-incubated in minimal media. The wild type laboratory strains of Saccharomyces boulardii (YM5016) and Saccharomyces cerevisiae (BJ5464-NpgA) are grown in synthetic minimal (SD) media. The S. cerevisiae TAN-1612-producing strain yPBA1474 and the S. boulardii TAN-1612-producing strain yPBA1407 are grown in SD-T (without tryptophan) and SD-UT (without uracil and tryptophan) media, respectively. Details of the TAN-1612-producing strains are provided in Table 29. The cells are grown overnight at 30° C. Caco-2 cells are seeded in 0.45 μm filter inserts placed in 24-well plates. The media for growing Caco-2 must not contain PenStrep or other antibiotics. The Caco-2 cells are grown for 4 days and media is changes as needed during this grow period.

TABLE 29
Strains used in this Example.

| | |
|---|---|
| yPBA1407 (S. | YM5016 ΔPEP4::pTDH3-NpgA-tENO2 ΔPRB1::pTDH3-NpgA- |
| boulardii TAN-1612 | tENO2 + pPBA-AV124_AmpR_Multigene-2μ-pCCW12-adaA_yeast- |
| producer) | codon-opt-tPGK1_pPGK1-adaB_yeast-codon-opt-tADH1_pTEF1- |
| | adaC_yeast-codon-opt-tENO1_pHSP26-adaD_yeast-codon-opt- |
| | tRPL15A |
| yPBA1474 (S. | BJ5464 ΔPEP4::pHSP26-Sb-NpgA-tENO2 ΔPRB1::pHSP26-Sb- |
| cerevisiae TAN- | NpgA-tENO2 + pPBA-AV124_AmpR_Multigene-2μ-pHSP26- |
| 1612 producer) | adaA_yeast-codon-opt-tPGK1_pPGK1-adaB_yeast-codon-opt- |
| | tADH1_pTDH3-adaC_yeast-codon-opt-tENO1_pHSP26-adaD_yeast- |
| | codon-opt-tRPL15A + pPBA-pAV116-pTDH3-Sb- |
| | ASPNIDRAFT_48051-yeast-cod_optimized-clean-tENO2 |

On Day 1, the yeast strains are inoculated in 100 mL of SD, SD-T or SD-UT media at OD600 ˜0.08. The yeast cells are grown for 3 days with shaking at 30° C.

On Day 4, the yeast cells were pelleted, washed and resuspended in mammalian cell media (DMEM with 15% FBS, 1 mM glutamine and 10 mM HEPES added). About 1-10 million yeast cells are transferred to each well of a 24-well plate. The filter inserts are moved to this plate so the yeast occupy the basolateral side and the mammalian Caco-2 cells are attached to the apical side of filters. Bay-11 (a potent anti-inflammatory drug) is added to some wells that contain wild-type yeast (i.e., that do not express TAN-1612) as positive controls. Each experiment is performed in triplicate. The cells are incubated for about 20-24 h.

On Day 5, LPS is added to every well to induce inflammation except for the negative control samples. The cells are incubated with LPS for about 20-24 hours.

On Day 6, the supernatant is collected from the apical side of the filter. The supernatant samples are centrifuged, aliquoted and frozen.

The level of inflammation for each supernatant sample is analyzed by detecting interleukin-8 (IL-8), which is used as an indicator of inflammation, by a commercial IL-8 ELISA detection kit. It is expected that the yeast cells that synthesize and secrete TAN-1612 reduces the inflammation induced by LPS.

### Example 12. Heterologous Catalysis of the Final Steps of Tetracycline Biosynthesis by Saccharomyces cerevisiae

The last steps in the biosynthesis of the tetracyclines are hydroxylation and reduction, starting from an anhydrotetracycline, catalyzed by an FAD-dependent anhydrotetracycline hydroxylase and an F420-dependent dehydrotetracycline reductase. In the biosynthesis of oxytetracycline (5-hydroxytetracycline), these steps are catalyzed by OxyS and OxyR, respectively, and in the biosynthesis of chlortetracycline (7-chlorotetracycline), these steps are catalyzed by CtcN and CtcM, respectively. Two key differences between the two pathways with regards to these last steps are the anhydrotetracycline structure from which they start and the number of hydroxylation steps on that anhydrotetracycline. While OxyS catalyzes two hydroxylation steps on anhydrotetracycline in the oxytetracycline pathway, CtcN catalyzes only one hydroxylation step on anhydrochlortetracycline in the chlortetracycline pathway. It was hypothesized that structural differences between OxyS and CtcN lead to a difference in the number of hydroxylation steps between the two enzymes.

A unique cofactor to the last steps of the tetracyclines' biosynthesis that is not native to S. cerevisiae is F420, a lactyl oligoglutamate phosphodiester derivative of 7,8-didemethyl-8-hydroxy-5-deazariboflavin (Fo). Fo is much more synthetically accessible than F420. As such, Fo can act as a substitute for F420 in some F420-dependent reactions in vitro, but it does not appear to have a redox role in living cells. F420-reducing NADPH dehydrogenase enzymes such as F420 NADPH oxidoreductase (FNO) from Archaeoglobus fulgidus can reduce F420 to its reducing agent active form, F420_{H2}.

In this Example, S. cerevisiae was used for the final steps of tetracycline biosynthesis, specifically the conversion of anhydrotetracycline to tetracycline, by the heterologous expression of OxyS, CtcM and FNO. This Example also discloses that synthetic Fo, exogenously supplied to the engineered S. cerevisiae strain can successfully replace F420 in this biosynthetic pathway. In addition, in this Example it is reported that the characterization of a proposed intermediate in oxytetracycline biosynthesis can explain structural differences between oxytetracycline and chlortetracycline. Tetracycline biosynthesis is enabled in this Example because when OxyS is expressed in S. cerevsiae, it performs just a single hydroxylation step on anhydrotetracycline and not two, as previously reported. Thus, this Example enhances the understanding of tetracycline biosynthesis and paves the road for total heterologous biosynthesis of tetracyclines in S. cerevisiae.

In order to carry out the final steps of a tetracycline biosynthesis in S. cerevisiae, namely the conversion of anhydrotetracycline to tetracycline, three enzyme types were heterologously expressed. The first, an anhydrotetracycline hydroxylase, converts anhydrotetracycline to dehydrotetracycline. In this Example, as in Example 1, OxyS from the oxytetracycline pathway was employed as the anhydrotetracycline hydroxylase. The second, a dehydrotetracycline reductase, converts dehydrotetracycline into tetracycline. Three dehydrotetracycline reductases, OxyR, DacO4 and CtcM from the oxytetracycline, dactylocycline and chlortetracycline pathways, respectively, were tested in a combinatorial approach. The third, an F420 reductase, reduces the cofactor used by the second type of enzyme. Here, three F420 reductases from Mycobacterium tuberculosis, Archeoglobus fulgidus and Streptomyces griseus were explored in a combinatorial approach as well. Commercially-available anhydrotetracycline was used as the substrate in this biosynthesis and synthetic Fo was used as a dehydrotetracycline reductase-cofactor instead of its much more complex derivative cofactor F420.

6-hydroxylation of anhydrotetracycline in Saccharomyces cerevisiae. The first step required to convert anhydrotetracyclines to tetracyclines is 6-hydroxylation. In the biosynthesis of oxytetracycline this step is catalyzed by OxyS. In order to test the capacity of S. cerevisiae to hydroxylate anhydrotetracycline, OxyS and anhydrotetracycline were as the enzyme-substrate pair (Scheme 1). This choice was made because OxyS functionally expresses in Escherichia coli and anhydrotetracycline is commercially available. The expression plasmid pSP-G1 was chosen in order to facilitate the coexpression of a dehydrotetracycline reductase enzyme, to append antibody tags to both the hydroxylase and the reductase and to constitutively express both enzymes. OxyS was cloned into pSP-G1 under the transcriptional control of the strong constitutive promoter TEF1 with a FLAG antibody tag at its C-terminus. Scheme 1 provided in FIG. 67 shows the hypothesized functional setup in the conversion of anhydrotetracycline to tetracycline in a +OxyS+CtcM+FNO yeast cell lysate in the presence of NADPH, Fo and G6P.

First, the catalysis of anhydrotetracycline hydroxylation by OxyS in S. cerevisiae cell lysate was tested by mass spectrometry. For the cell lysate experiment, +OxyS cells or their control −OxyS cells were supplied with the anhydrotetracycline starting material as well as with NADPH, a cofactor of OxyS. When a lysate of S. cerevisiae cells expressing OxyS is incubated with anhydrotetracycline, the molecular ion corresponding to 5a(11a)-dehydrotetracycline (2a, [M+H]+=443.3) has 4 times higher counts compared to the molecular ion corresponding to anhydrotetracycline ([M+H]+=427.3). As expected, the opposite is the case when a lysate of S. cerevisiae cells not expressing OxyS is incubated with anhydrotetracycline, where the molecular ion counts corresponding to anhydrotetracycline are 37 times higher than molecular ion counts corresponding to 5a(11a)-dehydrotetracycline (2a, FIG. 3).

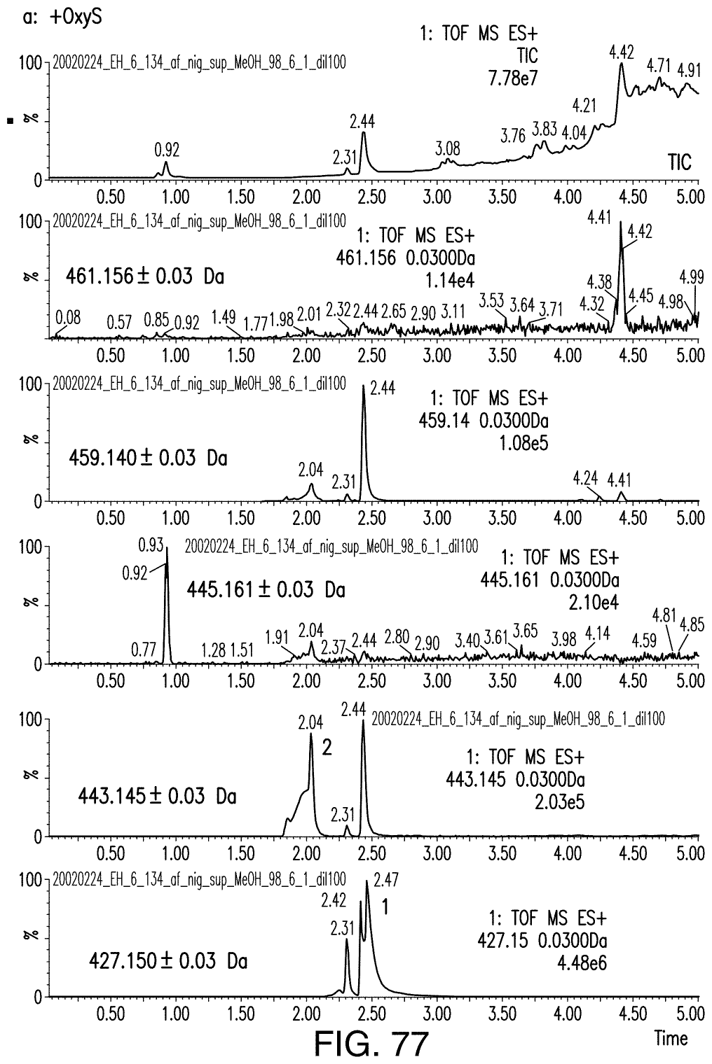

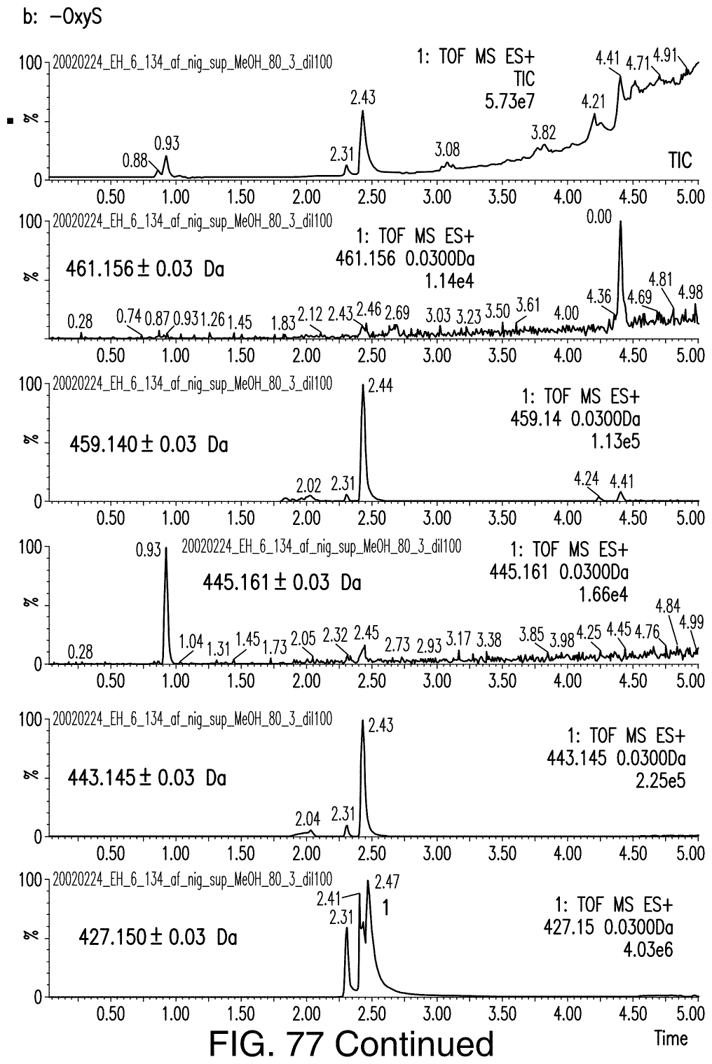

Following this cell lysate result, the hydroxylation of anhydrotetracycline by S. cerevisiae cells expressing OxyS was tested by incubation of whole cells with anhydrotetracycline. Cultures of OxyS expressing cells and control cells were pelleted and the pellets were resuspended and incubated overnight with anhydrotetracycline in Tris buffer (pH 7.45) prior to spectroscopic measurements. Indeed, the molecular ion corresponding to dehydrotetracycline had over 10 times higher ion counts in the OxyS expressing strain relative to the control strain (FIG. 77).

Next, the hypothesis that anhydrotetracycline is converted to the hydroxylation intermediate, 5a(11a)-dehydrotetracycline (2a), was tested by a larger scale reaction, purification, and NMR analysis of the isolates. A cell lysate of the +OxyS strain was incubated overnight with anhydrotetracycline and the hydroxylation product was isolated by liquid-liquid extraction using ethyl acetate and water followed by reverse phase HPLC purification. To prevent acidic and thermal degradation of the hydroxylation intermediate, reverse phase HPLC was performed with a mobile phase gradient of acetonitrile in Tris buffer (pH 7.45), and NMR analysis was performed at 273±5° K in methanol-d4. Attempts with other mobile phase gradients such as acetonitrile in NH₄OAc, H₂O:TFA, H₂O, as well as ambient temperature NMR after the use of these aqueous phases led to degradation of the intermediate.

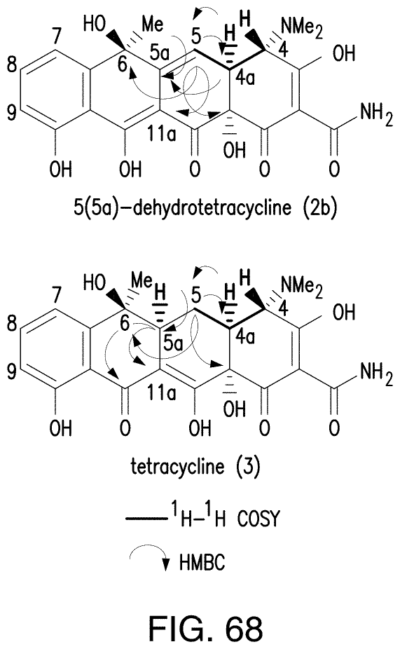

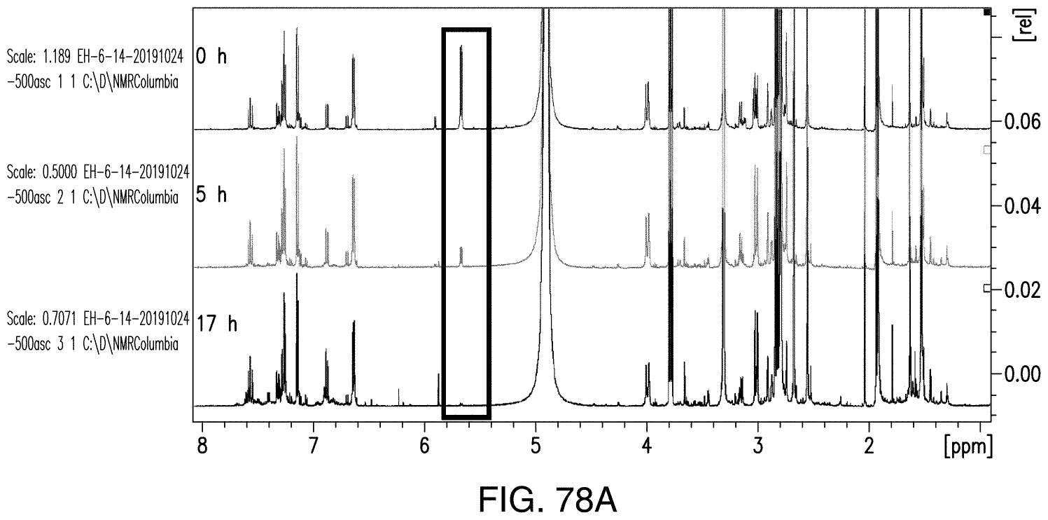

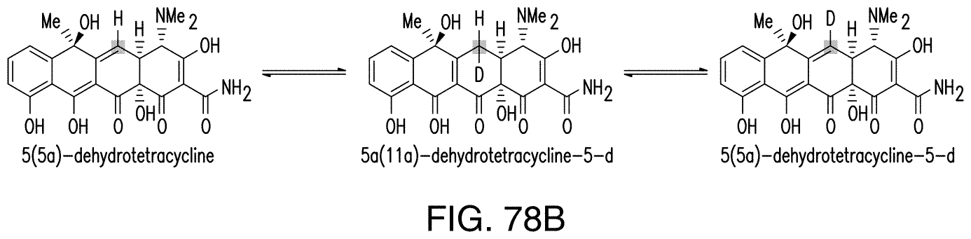

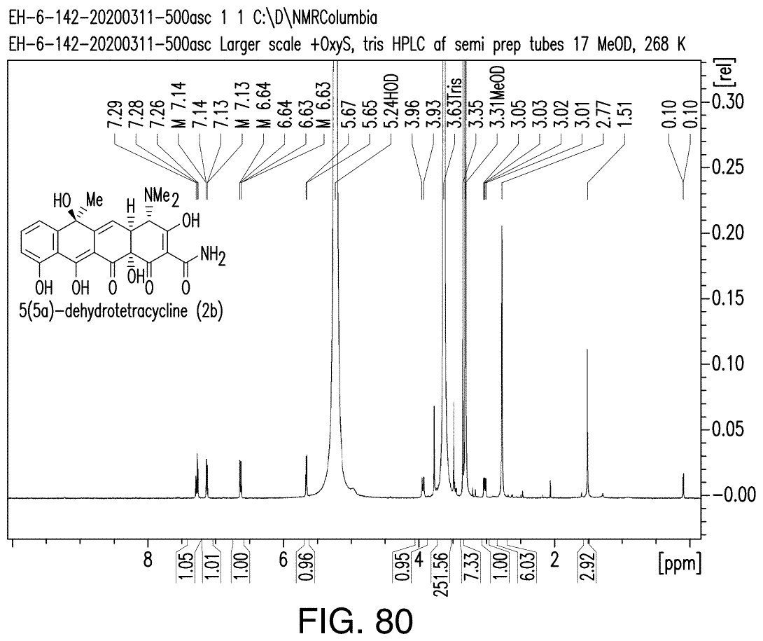

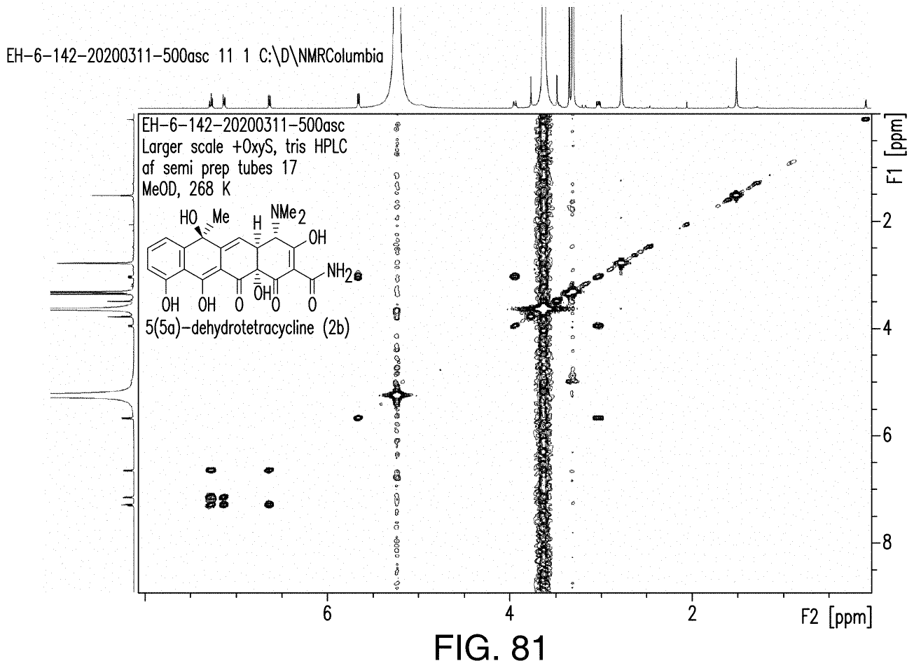

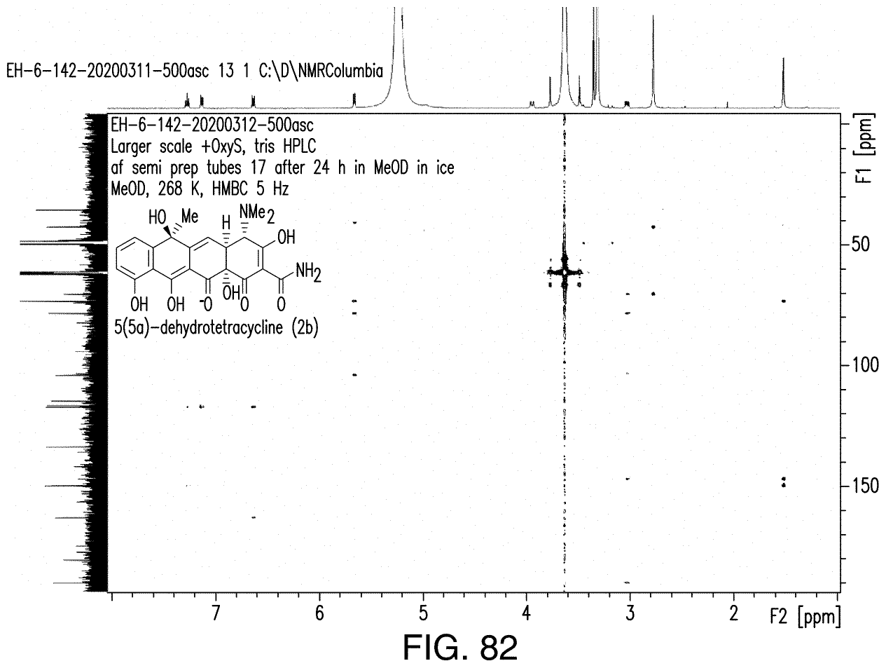

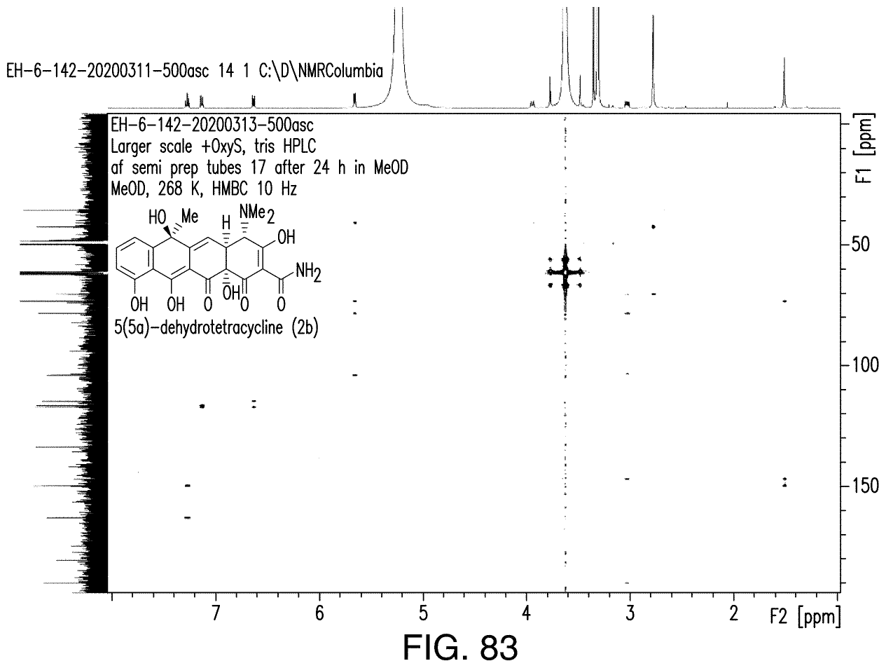

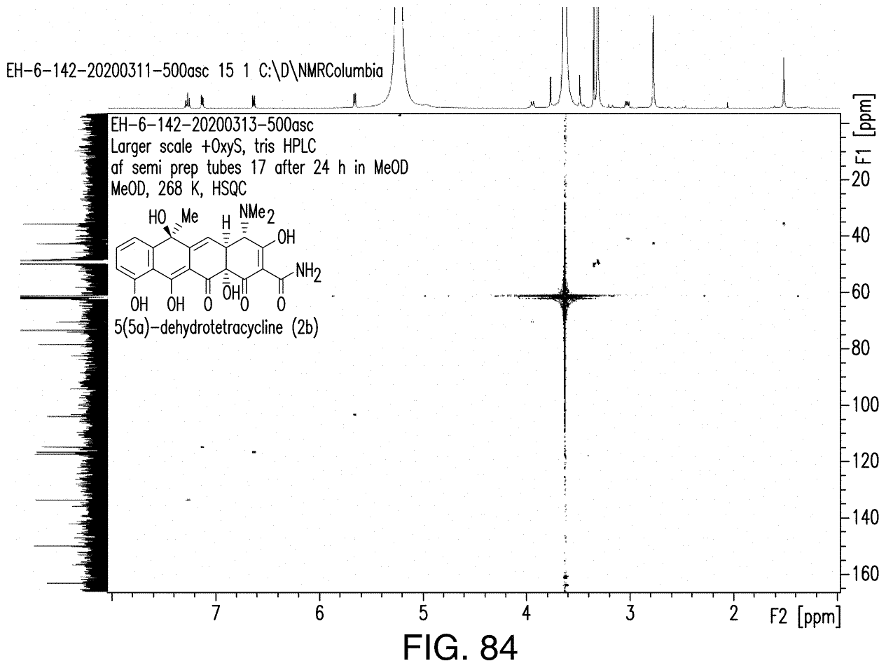

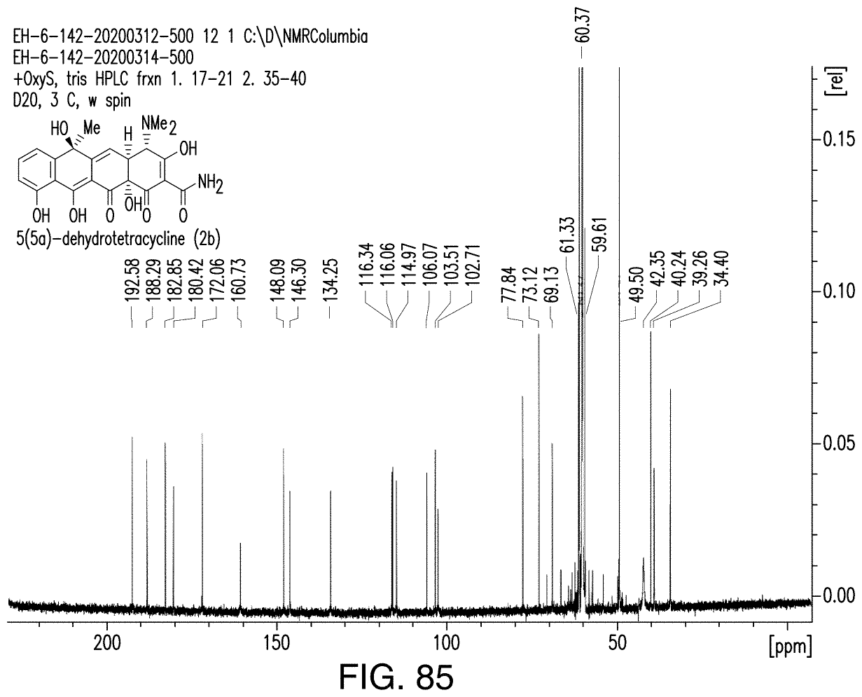

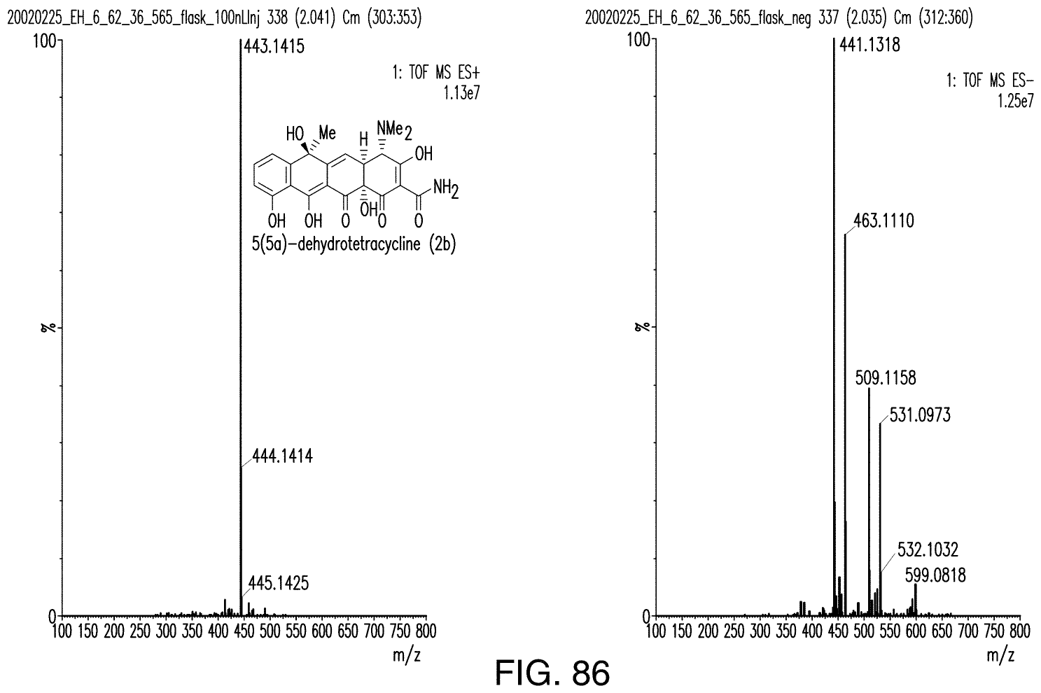

Notably, after the reaction of the +OxyS cell lysate with anhydrotetracycline, 5(5a)-dehydrotetracycline (2b) was obtained instead of the dehydrotetracycline isomer that was anticipated, 5a(11a)-dehydrotetracycline (2a, FIG. 68). All protons of 5(5a)-dehydrotetracycline (2b) have chemical shifts within 0.3 ppm of the corresponding protons in tetracycline except for the protons attached to the C5 and C5a positions (Table 30). As expected, a proton on C5a exists in tetracycline but not in 5(5a)-dehydrotetracycline (2b). In addition, while tetracycline has two aliphatic protons on C5 appearing at 6 1.93 and 2.22, 5(5a)-dehydrotetracycline (2b) has only one C5 proton at 6 5.66, typical of olefinic protons. By contrast, 5a(11a)-dehydortetracycline, the expected product of OxyS hydroxylation of anhydrotetracycline, should have two aliphatic protons on C5 and no additional olefinic proton (Scheme 1). The interconversion between 5(5a)-dehydrotetracycline (2b) and 5a(11a)-dehydrotetracycline (2a) is supported by a D-H exchange experiment, where the 5-H peak is eliminated over time at 300 K (FIG. 78) in methanol-d4. Despite this interconversion, only the 5(5a)-dehydrotetracycline isomer (2b), and not the 5a(11a)-dehydrotetracycline form (2a), was observed in the ₁H and ₁₃C NMR spectra, implying that 5(5a)-dehydrotetracycline (2b) is the prevalent isomer (Scheme 1). The ₁H and ₁₃C assignments of 5(5a)-dehydrotetracycline (2b) are supported by one- and two-dimensional NMR experiments (FIG. 68 and FIGS. 80-86).

TABLE 30
¹H-NMR data for 5(5a)-dehydrotetracycline (2b)
and for tetracycline (methanol-d4, 500 MHz)

| | 5(5a)- | |
|---|---|---|
| | dehydrotetracycline | Tetracycline (3) |
| no. | (2b) [symbol] in Hz) | [symbol] (J in Hz) |
| 4 | 3.03, dd (11.3) | 4.08, d (3.0) |
| 4a | 3.03, (11.5, 6.2) | 2.95, ddd (12.7, 3.0, 2.5) |
| 4-NMe₂ | 2.77, s | 3.03, s |
| 5 | 5.66, d (6.2) | 2.22 dddd |
| | | (13.4, 3.0, 3.0, 2.5); |
| | | 1.93 ddd |
| | | (13.4, 13.3, 11.2) |
| 5a | — | 3.05, m |
| 6-Me | 1.51, s | 1.63, s |
| 7 | 7.13, d (7.8) | 7.17, dd (7.8, 0.7) |
| 8 | 7.28, dd (8.0, 7.9) | 7.54, dd (8.1, 8.0) |
| 9 | 6.63, d (8.1) | 6.94, dd (8.4, 0.7) |

[symbol]

indicates data missing or illegible when filed

Reduction of the hydroxylation intermediate using Saccharomyces cerevisiae. The second step in the biosynthesis of tetracycline from anhydrotetracycline is to reduce the 5a(11a) α, β-unsaturated double bond (Scheme 1). The reduction at the 5a(11a) bond is known to be essential to the antibiotic activity of the tetracyclines. For example, 7-chlorotetracycline is over 20 times more potent than 7-chloro-5a(11a)-dehydrotetracycline against Staphylococcus aureus. To execute the reduction step, OxyR, the reductase of 5a(11a)-dehydrooxytetracycline from the oxytetracycline pathway, was placed under the control of PGK1 promoter in the pSP-G1-OxyS plasmid.

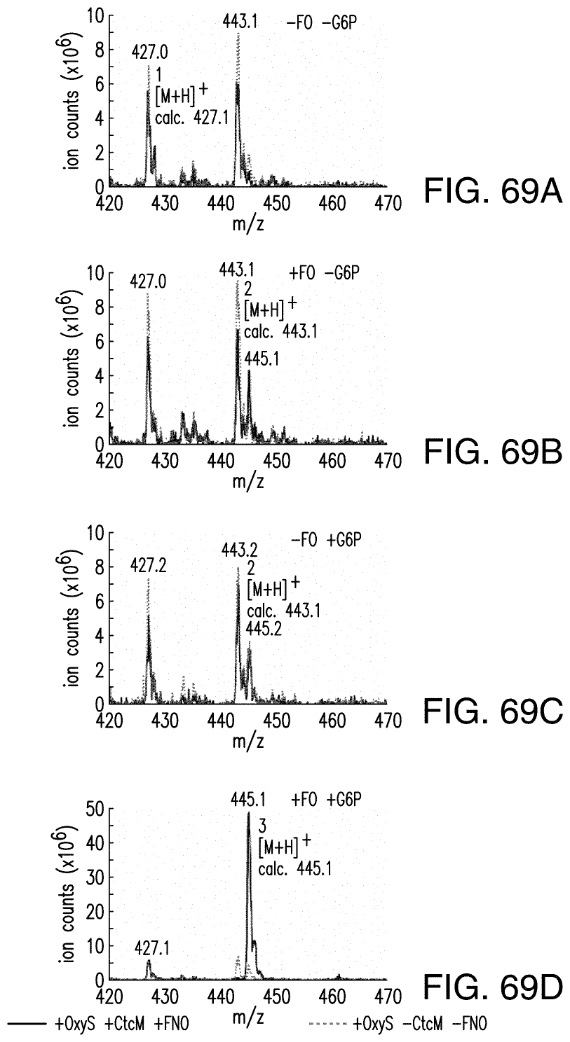

The catalytic activity of OxyR is known to be dependent on cofactor F420, a unique cofactor not native to S. cerevisiae. Fo is an intermediate in cofactor F420 biosynthesis and is known to successfully replace cofactor F420 as a substrate of F420 reductase enzymes with similar Km and kcat values. For example, a cofactor F420 reductase from Methanobacterium thermoautotrophicum had a Km value of 19 μM with F420 and a Km value of 34 μM with Fo. In another example, a cofactor F420 reductase from Methanococcus vannielii catalyzed the reduction of F420 and Fo with kcat/Km values of 158 and 56 min₋₁ μM₋₁, respectively. To test if OxyR could be functional in S. cerevisiae with Fo as a cofactor, synthetic Fo was exogenously added to a lysate of S. cerevisiae cells expressing OxyS, OxyR and an Fo reductase. It was tested whether F420 reductases can function as Fo reductases in this system. Towards this end, F420 reductases from three hosts, M. tuberculosis, A. fulgidus and S. griseus, were used by expression under the control of pGPD (pTDH3) on pRS413. The reaction mixture containing the S. cerevisiae cell lysate in Tris buffer (pH 7.45) also contained anhydrotetracycline, glucose, and NADPH. In the reactions with F420 reductase from M. tuberculosis, glucose-6-phosphate (G6P), the reducing agent used by this enzyme, was also included for Fo reduction. Since the other two F420 reductases use NADPH as the reducing agent of F420, and given that NADPH was already included in the reaction setup as a cofactor for OxyS, no additional reducing agent was added to the reactions of F420 reductases from A. fulgidus and S. griseus. It was found that the levels of the molecular ion peak corresponding to tetracycline ([M+H]+=445.2), the reduction product of 5a(11a)-dehydrotetracycline (2a, [M+H]+=443.2), increased when G6P is added (FIG. 69A vs FIG. 69C, FIG. 5B). Contrary to the expectation that G6P serves as a substrate for the Fo reductase, we found that neither Fo, the Fo reductase, nor OxyR contributed to the increase in the levels of the molecular ion peak corresponding to tetracycline either in the presence of G6P.

Given that OxyS performs one hydroxylation in S. cerevisiae (FIG. 67) as opposed to two in vitro and in S. rimosus, it was logical to test an alternative dehydrotetracycline reductase to OxyR. The native substrate for OxyR is hypothesized to be the doubly hydroxylated 5a(11a)-dehydrooxytetracycline (4) and not the singly hydroxylated 5a(11a)-dehydrotetracycline (2a, Scheme 1). Furthermore, the use of the alternative enzyme CtcM from the chlortetracycline pathway instead of OxyR has yielded in vitro an increased ratio of tetracycline to oxytetracycline. Another reasonable alternative candidate to OxyR was DacO4 from the dactylocycline pathway, an additional OxyR homolog, whose hypothetical native substrate, 5a(11a)-dehydrodactylocyclinone, is also not hydroxylated at C5. Therefore, CtcM and DacO4 were tested as alternative dehydrotetracycline reductases along with synthetic Fo and an F420 reductase from M. tuberculosis, A. fulgidus or S. griseus in a combinatorial approach. Gratifyingly, incubating the cell lysate of the strain encoding OxyS, CtcM and FNO from A. fulgidus with anhydrotetracycline resulted in an Fo-dependent peak corresponding to tetracycline (FIG. 69B vs FIG. 69A, solid line). As expected, such Fo-dependent increase in the peak corresponding to tetracycline was not observed in the control strain lacking CtcM and FNO (FIG. 69B vs FIG. 69A, dotted line).

In light of the G6P-dependent increase of the molecular ion counts corresponding to tetracycline even in the absence of CtcM (FIG. 5B), a potential synergy between Fo and G6P was tested. The cell lysate of the +OxyS+CtcM+FNO strain was incubated with anhydrotetracycline and NADPH in the presence of both Fo and G6P in Tris buffer (pH 7.45). Indeed, a major decrease in the molecular ion counts corresponding to the hydroxylated intermediate and a major increase in the molecular ion counts corresponding to tetracycline were observed (FIG. 69D vs FIG. 69A-C, solid line). As expected, this result was not observed in the +OxyS −CtcM −FNO cell lysate (FIG. 69D vs FIG. 69A-C, dotted line).

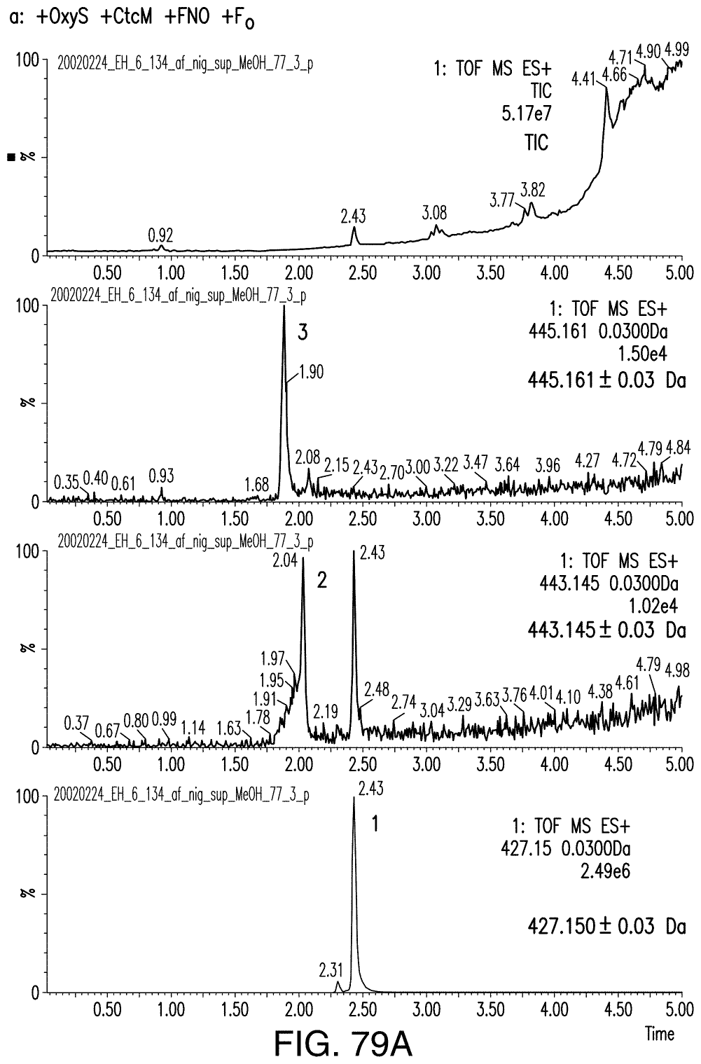

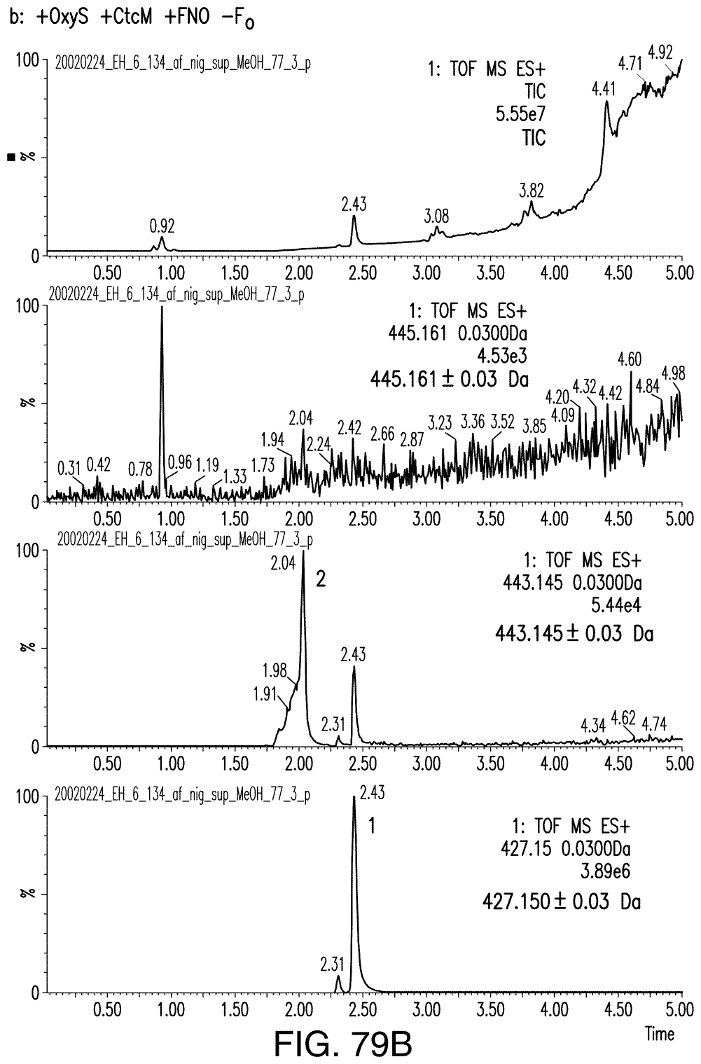

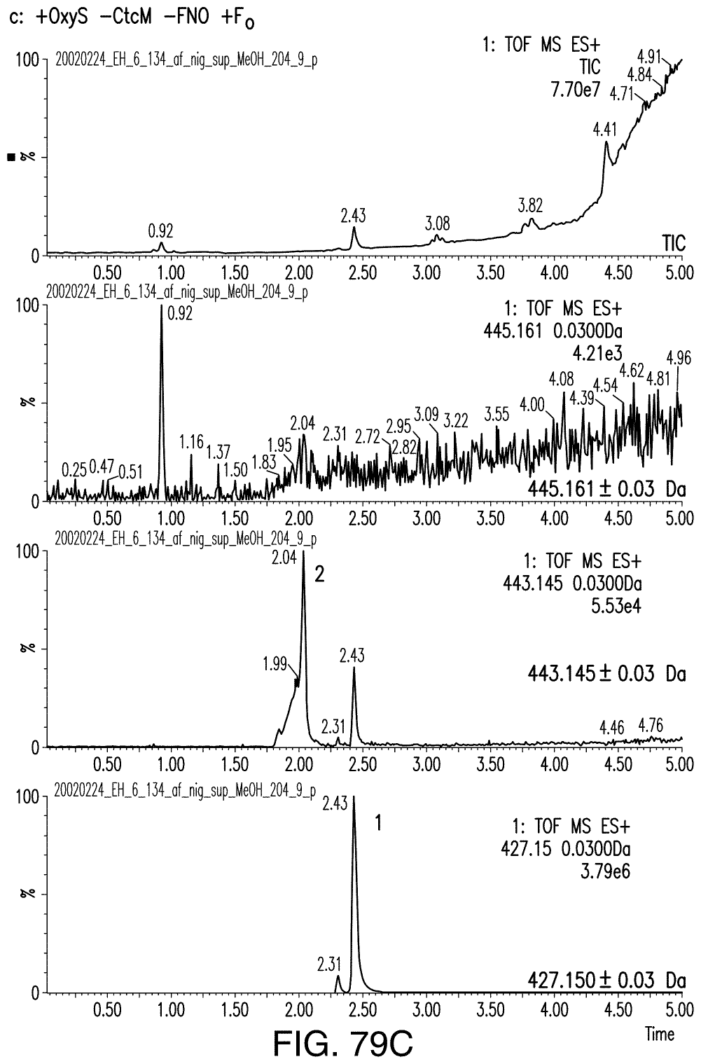

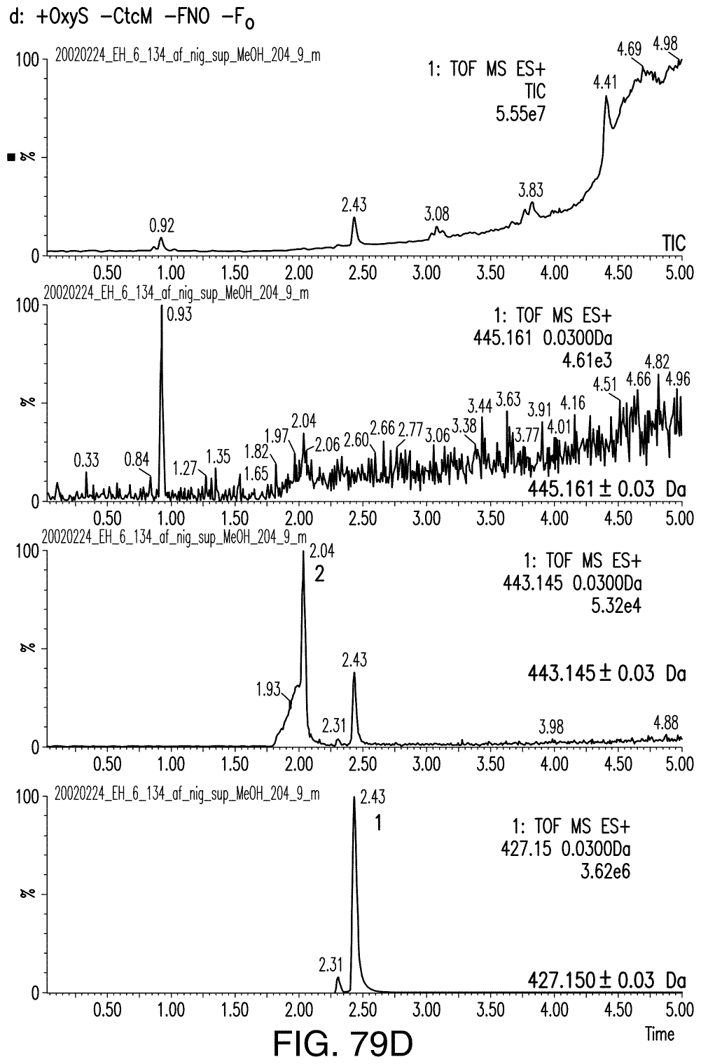

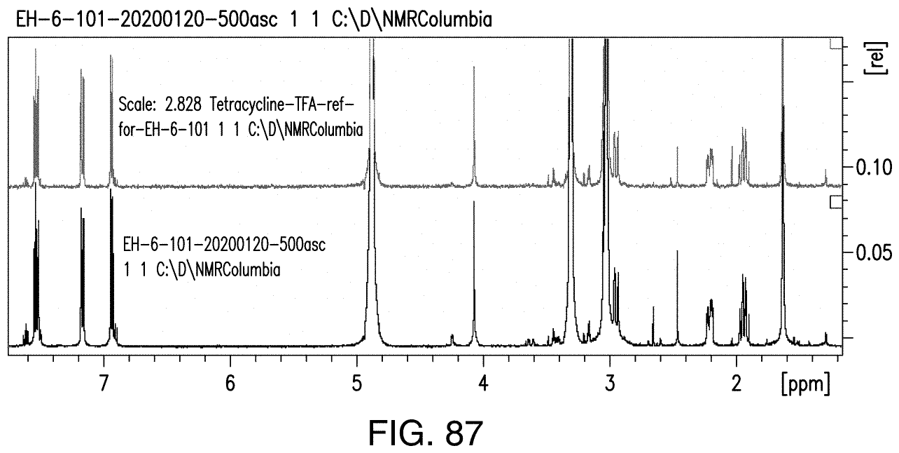

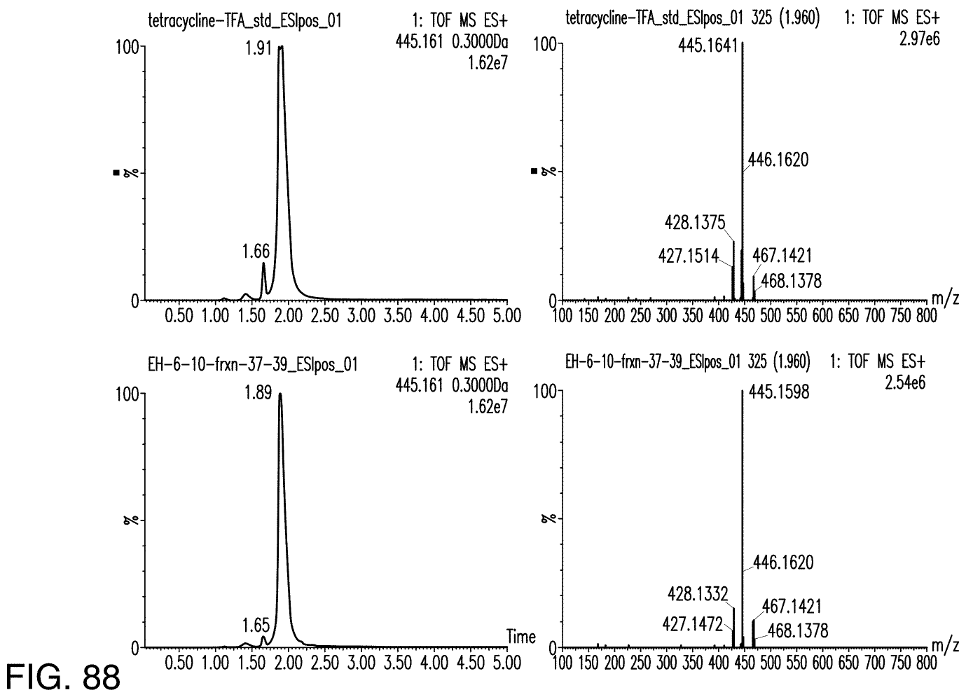

The conversion of anhydrotetracycline to tetracycline by the +OxyS+CtcM+FNO cell lysate in the presence of Fo and G6P was confirmed by purification and NMR characterization. Tetracycline was isolated in 24% yield using liquid-liquid extraction with ethyl acetate and water followed by reverse-phase HPLC separation using a mobile phase gradient of acetonitrile in H₂O:TFA (99.1:0.1). The identity of the tetracycline thus obtained to a tetracycline standard was confirmed by ₁H NMR and HRMS (FIGS. 87-88). In addition, the conversion of anhydrotetracycline to tetracycline upon incubation of anhydrotetracycline and Fo with unlysed yeast cells expressing OxyS, CtcM and FNO in Tris buffer is supported by mass spectrometry (FIG. 79).

This study demonstrates the use of S. cerevisiae for the final steps of tetracycline biosynthesis, the hydroxylation of anhydrotetracycline and the reduction of dehydrotetracycline (Scheme 1). These steps are key towards the total biosynthesis of tetracyclines in S. cerevisiae in an effort to biosynthesize new tetracycline analogs to combat antibiotic resistance. Heterologous biosynthesis of oxytetracycline was recently shown in Streptomyces lividans K4-11, a much closer relative of the original oxytetracycline biosynthesis host, Streptomyces rimosus. The choice of S. cerevisiae as a heterologous host in this study enabled the biosynthesis of tetracycline, instead of oxytetracycline. This difference resulted from two reasons: First, a single hydroxylation catalyzed by OxyS from the oxytetracycline pathway in S. cerevisiae, as opposed to a double hydroxylation in Streptomyces and in vitro (FIG. 67); Second, the use of CtcM from the chlortetracycline pathway, instead of OxyR from the oxytetracycline pathway, as the dehydrotetracycline reductase (FIG. 69).

This Example is the first to isolate and analyze a dehydrotetracycline intermediate when OxyS is expressed in the absence of a dehydrotetracycline reductase, such as OxyR or CtcM (FIG. 67, FIG. 68). The reaction product of OxyS rapidly degraded both in vitro, when OxyR was not concurrently used, and in vivo, in ΔoxyR S. lividans. Two explanations are noted regarding the ability to isolate and analyze 5(5a)-dehydrotetracycline (2b) from the +OxyS S. cerevsiae cell lysate. First, the additional degradation pathways that become possible in the presence of the additional 5-hydroxy that is installed by OxyS in vitro and in Streptomyces but not in this study. For example, anhydrooxytetracycline (5-hydroxyanhydrotetracycline) is known to undergo B-ring scission degradation, which is not possible in anhydrotetracycline. Second, in this Example the hydroxylated product of anhydrotetracycline was protected from acid, while in the in vitro study of OxyS acidic organic extraction was employed. Anhydrooxytetracycline is known to undergo B-ring scission specifically in the presence of dilute acid.

This characterization of 5(5a)-dehydrotetracycline (2b) enhances the current understanding of the last steps of chlortetracycline and oxytetracycline biosynthesis and the differences between these pathways. First, prior to this study, 5(5a)-dehydrotetracycline (2b) was an uncharacterized hypothetical intermediate in the OxyS hydroxylation of anhydrotetracycline. By characterizing 5(5a)-dehydrotetracycline (2b), this study supports the hypothesized mechanism of OxyS hydroxylation in the biosynthetic pathway of oxytetracycline (5, Scheme 1, FIG. 68). Second, prior to this study it was hypothesized that the double hydroxylation performed by OxyS as opposed to the single hydroxylation performed by CtcN, results from structural differences between OxyS and

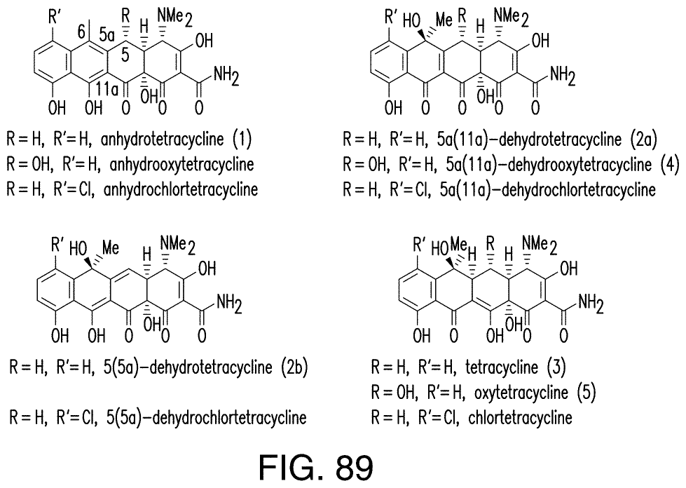

CtcN. An increased stability of 5(5a)-dehydrotetracycline (2b) over 5a(11a)-dehydrotetracycline (2a), supported by this study (Scheme 1, FIG. 68), can promote an additional hypothesis. Namely, that structural differences between the dehydrotetracycline intermediates of the two pathways can also contribute to the difference in the number of hydroxylations between the two pathways. Specifically, 5(5a)-dehydrotetracycline (2b) is the substrate for a second hydroxylation step and hence its increased stability over 5a(11a)-dehydrotetracycline (2a), the substrate for reduction, can promote a second hydroxylation (Scheme 1). Third, among the dehydrotetracycline intermediates in the biosynthesis of chlortetracycline, oxytetracycline and tetracycline, the only dehydrotetracycline that was previously characterized is 5a(11a)-dehydrochlortetracycline (2a, FIG. 89). Future studies can determine whether the lack of the 7-chloro substituent can be a contributing factor to an increased stability of 5(5a)-dehydrotetracycline (2b) over 5a(11a)-dehydrotetracycline (2a).

Furthermore, it has been shown that Fo, a synthetically accessible precursor in the biosynthesis of cofactor F420, can successfully replace F420 in the biosynthesis of tetracycline from anhydrotetracycline (FIG. 69). To function effectively in this setup, Fo needed to be accepted as a substrate both by the Fo reductase FNO and by the dehydrotetracycline reductase CtcM. The ability of Fo to replace F420 in enzymatic reactions of F420 reductases was previously known. However, this Example critically shows that this ability further extends to F420-dependent biosynthetic enzymes such as CtcM from the chlortetracycline pathway. This result is important since F420 is a unique cofactor that is not native to common heterologous hosts such as S. cerevisiae and E. coli. Moreover, future studies can incorporate the biosynthesis of Fo into a tetracycline biosynthesis system in S. cerevisiae since Fo is only one biosynthetic step removed from common S. cerevisiae metabolites, tyrosine and 5-amino-6-ribitylamino-2,4(1H,3H)-pyrimidinedione. This approach of using Fo to replace F420 in the heterologous biosynthesis of tetracyclines could be further extended to other F420-dependent pathways as well, such as the pathways leading to lincosamides and aminoglycosides.

Without being limited to a particular theory, the synergistic effect of Fo and G6P could be explained by a G6P-dependent increase of NADPH pools from NADP+, catalyzed by G6P dehydrogenase, leading to the reduction of Fo to FoH₂ (Scheme 1b). Interestingly, G6P increases the ion counts corresponding to protonated tetracycline also in the absence of Fo, albeit to a lesser degree than in the presence of Fo (FIG. 69). Future research can determine whether a native S. cerevisiae enzyme is catalyzing the reduction of 5a(11a)-dehydrotetracycline (2a) in the absence of Fo in a G6P-dependent manner. Such G6P dependence could be direct or through an increase in another cellular reducing agent as NADPH.

Beyond the use of S. cerevisiae towards tetracycline biosynthesis, S. cerevisiae can offer access to novel tetracycline analogs. This opportunity exists because of the widely available tools for genetically modifying S. cerevisiae as well as enhanced accessibility of biosynthetic enzymes such as P450s. Specifically, S. cerevisiae can be used to express alternative hydroxylases such as DacO1 and CtcN that proved previously to be insoluble when expressed in other heterologous hosts such as E. coli and Streptomyces. Such enzymes can hydroxylate alternative anhydrotetracycline substrates, as well as lead to hydroxylated products of the opposite stereochemistry in the 6-position, thereby covering additional chemical space. Importantly, such chemical space is not covered by existing methods of synthesizing tetracyclines, despite the promise of 6-position tetracycline analogs for potent antibiotic activity. Moreover, the use of S. cerevisiae for the conversion of anhydrotetracyclines to tetracyclines can readily utilize fungal anhydrotetracycline analogs for further diversity generation. Finally, semisynthesis could be used to decorate such tetracyclines produced by S. cerevisiae to generate a diversity of novel tetracycline analogs with the potential to combat existing and future modes of antibiotic resistance.

TABLE 31
Chemical formulas and expected masses for the ions of Compounds 1-5.

| Compound no. | [M + H]⁺ formula | [M + H]⁺ mass | [M − H]⁻ formula | [M − H]⁻ mass |
|---|---|---|---|---|
| 1 | C₂₂H₂₃N₂O₇ | 427.1500 | C₂₂H₂₁N₂O₇ | 425.1354 |
| 2 | C₂₂H₂₃N₂O₈ | 443.1449 | C₂₂H₂₁N₂O₈ | 441.1303 |
| 3 | C₂₂H₂₅N₂O₈ | 445.1605 | C₂₂H₂₃N₂O₈ | 443.1460 |
| 4 | C₂₂H₂₃N₂O₉ | 459.1398 | C₂₂H₂₁N₂O₉ | 457.1253 |
| 5 | C₂₂H₂₅N₂O₉ | 461.1555 | C₂₂H₂₃N₂O₉ | 459.1409 |

*Compound 2a and 2b are of the same formula and therefore referred to collectively here as 2.

Methods Used in this Example:

General methods. Absorption and fluorescence spectra were recorded on Infinite-M200 fluorescent spectrometer. DNA sequences were purchased from IDT. Polymerases, restriction enzymes and Gibson Assembly mix were purchased from New England Biolabs. Sanger sequencing was performed by Genewiz. Yeast strains were grown at 30° C. and shaker settings were 200 rpm, unless otherwise indicated. Yeast transformations were done using the lithium acetate method. Plasmids were cloned and amplified using Gibson Assembly and cloning strain C3040 (New England Biolabs). Unless otherwise indicated, yeast strains were grown on synthetic minimal media lacking histidine and/or uracil and/or tryptophan and/or leucine, as indicated by the abbreviation HUTL. Yeast strain patches were obtained from glycerol stocks by streaking on an agar plate of synthetic medium lacking the appropriate amino acid markers, incubating at 30° C. for 3 days, patching single colonies onto a fresh agar plate and incubating at 30° C. overnight. Protein homology was calculated by BLAST (https://blast.ncbi.nlm.nih.gov) using the standard sequence alignment parameters. DataExpress was used to analyze Advion CMS data and MassLynx was used to analyze Waters XEVO QTOF data.

Codon optimization by COOL (http://cool.syncti.org/index.php) was used for all hydroxylases and reductases unless noted explicitly that JCAT or no optimization was used (http://www.jcat.de/). Optimization parameters chosen were individual codon use, codon context GC content of 39.3% and S. cerevisiae organism. The following restriction sites were generally excluded: GAGACC, GGTCTC, GGATCC, GAGCTC, CTCGAG, GAAGAC, GTCTTC, CGTCTC, GAGACG, GAATTC, TTAATTAA, TCTAGA, ACTAGT, CCCGGG, CTGCAG, AAGCTT, GTCGAC, ACGCGT, GGTACC, GCGGCCGC, AGATCT, GGCCGGCC, CCGCGG, GCTAGC and CCATGG.

Preparative HPLC was carried out with a C-18 5μ column, 250×10 mm, eluent given in parentheses. NMR spectra were obtained using Bruker 400 MHz or 500 MHz instruments, as indicated. Unless stated otherwise, mass spectroscopy measurements were performed on Advion CMS mass spectrometer equipped with atmospheric pressure chemical ionization (APCI) source. HRMS spectra and MS analyses of whole cell supernatants were taken on a Waters ACQUITY UPLC XEVO QTOF equipped with a BEH C18 column (2.1×50 mm) at 30° C. with a flow rate of 0.8 mL/min. Unless stated otherwise, all reagents, salts and solvents were purchased from commercial sources and used without further purification.

General protocol for hydroxylation/reduction assay with cell lysate. Fresh patches of strains harboring the plasmid for the hydroxylation with/without reduction enzyme and with/without the plasmid for the F420 reductase enzyme and control strains were inoculated in 5 mL selective media (U- or HU-) in 15 mL culture tubes (Corning 352059) and placed in shaker overnight to OD600 2-3. Overnight cultures were used to inoculate 100 mL selective media (U- or HU-) cultures in 500 mL conical flasks with a starting OD of 0.01-0.05. Cells were grown to final OD of 0.6-0.8 before pelleting in 2 50 mL tubes (Corning 352098) at 4° C., 4000 rpm for 20 min. Each pellet was redissolved in 0.5 mL H₂O and the suspension was distributed into two presterilized 1.5 mL Eppendorf tubes and pelleted at 14,000 rpm for 10 min at 4° C. Pellets were stored at −20° C. prior to further use.

Pellets were weighed and thawed on ice. A 99:1 mixture of Y-PER yeast protein extraction reagent (ThermoFisher Scientific 78991) and HALT protease inhibitor cocktail (ThermoFisher Scientific PI87786) was added in a ratio of 3 μL mixture per mg pellet and placed on orbital shaker for 20 min at 22° C., followed by 10 min centrifugation at 14000 rpm at 4° C. and the cell lysate was transferred to a new 1.5 mL Eppendorf tube, kept on ice and used within 1 h.

The cell lysate (0.080 mL) was added as the last component to a 4 mL vial (Chemglass CG-4900-01) containing 0.280 mL of 143.0 mM Tris (pH 7.45), 7.7 mM anhydrotetracycline HCl (AdipoGen CDX-A0197-M500), 4.3 mM NADPH tetrasodium hydrate (Sigma-Aldrich N7505), 26.4 mM mercaptoehtanol, 0.5 mM of FO (in experiments labeled +FO, 0 mM in all other experiments), 14.3 mM glucose-6-phosphate (in experiments labeled +G6P, 0 mM glucose-6-phosphate in all other experiments) and 0.040 mL glucose (278.0 mM) for final concentrations of 100.0 mM Tris, 5.4 mM anhydrotetracycline HCl, 3 mM NADPH, 18.5 mM mercaptoethanol, 0 or 0.4 mM FO as indicated, 0 or 10.0 mM G6P as indicated and 27.8 mM glucose. A septum was placed on top of the vial through which a needle was inserted to allow air exchange and the reaction was left at 22° C. overnight. After night, 1 mL of MeOH was added, the contents were mixed and the reaction was filtered through a PTFE 0.2 μm filter (Acrodisc 4423) prior to analysis by mass and UV/Vis spectrometry.

Protocol for hydroxylation/reduction assay with whole cells. Fresh patches of strains harboring the plasmid for the hydroxylation and/or reduction enzyme and control strains were inoculated in 5 mL selective media (U- or HU-) in 15 mL culture tubes (Corning 352059) and placed in shaker overnight to OD600 2-3. Overnight cultures were used to inoculate 100 mL selective media (U- or HU-) cultures in 500 mL conical flasks with a starting OD of 0.05-0.1. Cells were grown for 22 h before being placed at 15° C. for an additional 10-12 h. Cells were then pelleted in 2 50 mL tubes (Corning 352098) at 10° C., 3500 rpm for 5 min. Each pellet was redissolved in 0.5 mL H₂O and the suspension was distributed into a presterilized 1.5 mL Eppendorf tube and pelleted at 11000 rpm for 3 min at 10° C. Pellets were placed on ice and used within 1 h.

When strains EH-3-98-6 and EH-3-80-3 were used, pellets from 50 mL culture were redissolved in H₂O (1.025 mL) and added as the last component to 15 mL culture tubes (Corning 352059) containing 1.100 mL of 8 mg/mL anhydrotetracycline HCl, 0.125 mL glucose solution in H₂O (40%) and 0.250 mL 1 M Tris buffer pH 7.45 and were placed in shaker at 350 rpm at 21° C. for 27 h. Cultures were then pelleted and the supernatant was diluted into H2O before being used for mass and UV/Vis spectroscopy.

When strains EH-6-77-3 and EH-3-204-9 were used, pelleted unlysed cells were redissolved in H₂O (0.4 mL) and added as the last component to 15 mL culture tubes (Corning 352059) containing 1.100 mL of 8 mg/mL anhydrotetracycline HCl, 0.125 mL glucose solution in H₂O (40%), 0.250 mL 1 M Tris buffer pH 7.45 and 0.625 mL 1.5 mM Fo (+FO) or 0.625 mL H2O (−FO) and were placed in shaker at 350 rpm at 21° C. for 27 h. Cultures were then pelleted and the supernatant was diluted into H2O before being used for mass and UV/Vis spectroscopy.

Biosynthesis of (5,5a)-dehydrotetracycline (2b) using S. cerevisiae. Fresh patches of EH-3-248-1 harboring the plasmid encoding OxyS were inoculated in 2×5 mL selective media (U-) in 15 mL culture tubes (Corning 352059) and placed in shaker overnight to OD600 2-3. Overnight cultures were used to inoculate 500 mL selective media (U-) cultures in 2 L conical flasks with a starting OD of 0.01-0.05. Cells were grown to final OD of 0.75 before pelleting in 500 mL tubes at 4° C., 6000 rpm. The pellet was redissolved in 25 mL H₂O and the suspension was distributed into 50 mL falcon tubes and pelleted at 4° C., 4000 rpm. The pellet was then transferred into four presterilized 1.5 mL Eppendorf tubes and pelleted at 14000 rpm for 10 min at 4° C. Pellets were stored at −20° C. prior to further use.

Pellets were weighed and thawed on ice. A 99:1 mixture of Y-PER yeast protein extraction reagent (ThermoFisher Scientific 78991) and HALT protease inhibitor cocktail (ThermoFisher Scientific P187786) was added in a ratio of 3 μL mixture per mg pellet and placed on orbital shaker for 20 min at 22° C., followed by 10 min centrifugation at 14000 rpm at 4° C. and the cell lysate was transferred to a new 1.5 mL Eppendorf tube, kept on ice and used within 1 h.

The cell lysate (3.2 mL) was added as the last component to a 50 mL round bottom flask with a stir bar containing 12.8 mL of 143.0 mM Tris (pH 7.45), 7.7 mM anhydrotetracycline HCl (AdipoGen CDX-A0197-M500), 4.3 mM NADPH tetrasodium hydrate (Sigma-Aldrich N7505), 26.4 mM mercaptoethanol, and 39.7 mM glucose for final concentrations of 100.0 mM Tris, 5.4 mM anhydrotetracycline HCl, 3.0 mM NADPH, 18.5 mM mercaptoethanol, and 27.8 mM glucose. A septum was placed on top of the flask through which a needle was inserted to allow air exchange and the reaction was left at 22° C. overnight. After 14 h, the aqueous mixture was extracted two times with EtOAc. The combined organic fraction was extracted with water. MeCN was then added to the combined aqueous phase and it was then dried at 20° C. The contents were then dissolved in a mixture of water, MeCN and MeOH and purified by preparative RP-HPLC (1-20% MeCN in Tris (pH 7.45, 20.0 mM, 60 min) to afford 5(5a)-dehydrotetracycline (2b) after lyophilization (11.1 mg, 25% yield) as a yellow solid. 2b: 1H NMR (500 MHz, methanol-d4): δ 7.28 (dd, J=8.0, 7.9 Hz, 1H), 7.13 (d, J=7.8, 1H), 6.63 (d, J=8.1 Hz, 1H), 5.66 (d, J=6.2 Hz, 1H), 3.95 (d, J=11.3 Hz, 1H), 3.03 (dd, J=11.5, 6.2 Hz, 1H), 2.77 (s, 6H), 1.51 (s, 3H). 13C NMR (500 MHz, D₂O) δ 192.6, 188.3, 182.9, 180.4, 172.1, 160.7, 148.1, 146.3, 134.3, 116.3, 116.1, 115.0, 106.1, 103.5, 102.7, 77.8, 73.1, 69.1, 42.4, 39.3, 34.4. HRMS (ES+): m/z calcd for C₂₂H₂₃N₂O₈+, 443.1449; found, 443.1415 [M+H]+. HRMS (ES−): m/z calcd for C₂₂H₂₁N₂O₈−, 441.1303; found, 441.1318 [M−]−.

Biosynthesis of tetracycline using S. cerevisiae. Fresh patches of EH-6-77-3 harboring the plasmids encoding OxyS, CtcM and FNO were inoculated in selective media (HU-) in 15 mL culture tubes (Corning 352059) and placed in shaker overnight to OD600 2-3. Overnight cultures were used to inoculate 100 mL selective media (HU-) cultures in 500 mL conical flasks with a starting OD of 0.01-0.05. Cells were grown to final OD of 0.75 before pelleting in 500 mL tubes at 4° C., 6000 rpm. The pellet was redissolved in 25 mL H₂O and the suspension was distributed into 50 mL falcon tubes and pelleted at 4° C., 4000 rpm. The pellet was then transferred into four presterilized 1.5 mL Eppendorf tubes and pelleted at 14000 rpm for 10 min at 4° C. Pellets were stored at −20° C. prior to further use.

Pellets were weighed and thawed on ice. A 99:1 mixture of Y-PER yeast protein extraction reagent (ThermoFisher Scientific 78991) and HALT protease inhibitor cocktail (ThermoFisher Scientific P187786) was added in a ratio of 3 μL mixture per mg pellet and placed on orbital shaker for 20 min at 22° C., followed by 10 min centrifugation at 14000 rpm at 4° C. and the cell lysate was transferred to a new 1.5 mL Eppendorf tube, kept on ice and used within 1 h.

The cell lysate (0.440 mL) was added as the last component to 4 borosilicate vials of 4 mL each (4×0.110 mL) containing 2.200 mL (0.550 mL each) of 143.0 mM Tris (pH 7.45), 7.7 mM anhydrotetracycline HCl (AdipoGen CDX-A0197-M500), 4.3 mM NADPH tetrasodium hydrate (Sigma-Aldrich N7505), 26.4 mM mercaptoethanol, 2.1 mM Fo, 143.0 mM G6P and 39.7 mM glucose for final concentrations of 100.0 mM Tris, 5.4 mM anhydrotetracycline HCl, 3.0 mM NADPH, 18.5 mM mercaptoethanol, 0.4 mM Fo, 100.0 mM G6P and 27.8 mM glucose. A septum was placed on top of each vial through which a needle was inserted to allow air exchange and the reaction was left at 22° C. overnight. After 16 h, the aqueous mixture was extracted two times with EtOAc (7.5 mL in each round). The combined organic fraction was extracted with water (7.5 mL). MeOH was then added to the combined aqueous phase and it was then dried at 25-30° C. The contents were then dissolved in a mixture of water, MeCN and MeOH and purified by preparative RP-HPLC (1-50% MeCN in 99.9%:0.1% H₂O/TFA, 90 min) to afford after drying tetracycline (1.8 mg, 24% yield) as a yellow solid with HRMS and 1H-NMR spectra identical to tetracycline standard (FIG. 88 and FIG. 89).

Strain and Plasmids. The strains and plasmids used in this Example are shown in Table 32 and 33, respectively.

Sequences. The sequences used in this Example are shown in Table 34. As shown in Table 34, the sequence for the hydroxylase OxyS from Streptomyces rimosus (GenBank AAZ78342.1) is shown capitalized within the context of the pSP-G1 backbone containing pTEF1, the FLAG tag and tADH1 (partial, uncapitalized). The sequences for the reductases OxyR, CtcM and DacO4 from Streptomyces rimosus, Kitasatospora aureofaciens and Dactylosporangium sp. SC14051 (GenBank: DQ143963.2, AEI98656.1 and JX262387.1, respectively) are shown capitalized within the context of the pSP-G1 backbone containing pPGK1, the myc tag and tCYC1 (partial, uncapitalized) (Table 34). Sequences for the three F420 reductases from M. tuberculosis, A. fulgidus and S. griseus are shown along with the pGPD (pTDH3) promoter capitalized within the context of the pRS413 backbone containing the tCYC1 terminator (partial, uncapitalized) (Table 34). The NCBI/Genbank reference sequences used for the Fo reductases from M. tuberculosis, A. fulgidus and S. griseus are CP023708.1, NC_000917.1 and NC_010572.1 (6172267 . . . 6172977) (Table 34). Prior to codon optimization, leucine codon in M. tuberculosis F420 reductases in subsequence CATTGAACAACACCCGGTTT (SEQ ID NO: 216) was changed to methionine so that the total sequence matches the protein sequence used for M. tuberculosis F420 crystallization (Table 34).

TABLE 32
Strains used in this Example.

| | Genotype | Source Reference |
|---|---|---|
| FY251 | MATα leu2-Δ63 ura3-52 his3-Δ200 | ATCC 96098 |
| BJS464- | MATα ura3-52 his3-Δ200 leu2-Δ1 | */(4) |
| NPgA | trp1 pep4::HIS3 δ:: pADH2-npgA-tADH2 prb1Δ1.6R can1 | |
| | GAL | |
| EH-3-80-3 | PY2S1 pSP-G1 | This Studv |
| EH-3-98-6 | FY-231 AL-1-101-010 | This Studv |
| EH-3-153-8 | PY-251 AL-1-119-A-C7 | This Study |
| EH-3-204-5 | FY-231 AL-1-119-A-C7 AL-215-D-C1 | This Study |
| EH-3-204-6 | FY-251 AL-1-119-A-C7 AL-255-Cl | This Study |
| EH-3-204-7 | FY-251 AL-1-119-A-C7, AL-235-C5 | This Study |
| EH-3-204-S | FY-251 AL-1-119-A-C7, pRS413-pGAL1 | This Study |
| EH-3-204-9 | FY-251 AL-1-101-C10, pRS413-pGAL1 | This Study |
| EH-3-248-1 | B35464-NpgA AL-1-101-C10 | This Study |
| EH-3-248-8 | BJ5464-NpgA pSP-G1 | This Study |
| EH-6-77-1 | FY-251 LR-23-C-C7, AL-2 15-D-C1 | This Study |
| EH-6-77-2 | FY-251 LR-23-A-C3. AL-215-D-C1 | This Study |
| EH-6-77-3 | FY-251 LR-23-C-C7. AL-255-C1 | This Study |
| EH-6-77-4 | FY-251 LR-23-A-C3, AL-255-C1 | This Study |
| EH-6-77-5 | FY-251 LR-23-C-C7 AL-235-C5 | This Study |
| EH-6-77-6 | FY-251 LR-23-4-03 AS-235-C5 | This Study |

TABLE 33
Plasmids used in this Example.

| | Description | Source/Reference |
|---|---|---|
| pSP-G1 | pTEFI-FLAG-tADH1, pPGK1-MYC-tCYC1,2 μ, Ura | Addgene 64736 |
| pRS413- | Shuttle vector (empty). His marker, CEN | ATCC 87326 |
| pGAL1 | | |
| AL-1-101-C10 | pTEF1-oxyS-tADH1 in pSP-G1 | This Studv |
| AL-1-119-A-C7 | pTEF1-oxyS-tADH1, pPGK1-oxyR-tCYC1 in pSP-G1 | This Study |
| AL-1-215-D-C1 | FGD1 from Mycobacterium tuberculosis | This Study |
| AL-1-255-Cl | F₄₂₀-dependent NADP+ oxidoreductase from Archeoglobus | This Study |
| | fulgidus | |
| AL-1-235-C5 | NADPH-dependent P₄₂₀ reductase from Streptomyces | This Study |
| | griseus | |
| LR-23-A-C3 | pTEF1-oxyS-tADH1, pPGK1-dacO4-tCYC1 in pSP-G1 | This Studv |
| LB-23-C-C7 | pTEF1-oxyS-tADH1, pPGK1-ctcM-tCYC1 in pSP-G1 | This Study |

TABLE 34
Sequences used in this Example.

| | |
|---|---|
| pTEFl-oxyS- | tcaagtttcagtttcatttttcttgttctattacaactttttttacttcttgctcattagaaagaaagcatagca |
| tADHl-m-ySP- | atctaatctaagttttaattacaagcggccgcATGAGGTACGATGTTGTTATAGCTGGTGCAGGACCCACCGGTT |
| G1-AL-1-101- | TGATGTTAGCATGTGAACTTCGGCTGGCGGGTGCCAGAACTTTGGTTTTAGAAAGATTAGCCGAGCCTGTTGACT |
| C10 and AL-1- | TCTCGAAAGCTCTAGGAGTCCACGCTCGCACTGTTGAACTATTAGATATGAGAGGCCTCGGTGAAGGATTCCAGG |
| 119-A-C7 | CTGAAGCACCAAAGTTAAGAGGTGGTAATTTTGCCTCATTAGGCGTCCCCCTGGATTTCTCATCATTTGATACTA |
| | GACACCCATATGCATTGTTTGTTCCACAAGTACGAACTGAAGAACTGCTAACAGGTAGAGCTTTGGAGCTAGGGG |
| | CGGAGCTGCGTCGTGGTCATGCCGTGACCGCCTTGGAACAAGATGCTGATGGTGTTACTGTGAGCGTGACAGGCC |
| | CTGAAGGCCCATACGAAGTAGAATGTGCTTATTTGGTGGGCTGCGACGGTGGAGGCAGTACGGTGAGGAAACTAT |
| | TGGGCATAGATTTTCCAGGTCAAGACCCACATATGTTTGCTGTCATCGCAGACGCAAGATTCAGAGAAGAGCTTC |
| | CTCACGGAGAAGGTATGGGTCCTATGCGTCCTTATGGTGTCATGAGACATGACCTTCGTGCATGGTTCGCAGCAT |
| | TTCCGCTAGAACCAGACGTCTACAGAGCAACAGTCGCCTTTTTCGATAGACCGTATGCTGACAGGAGGGCGCCGG |
| | TAACGGAGGAGGATGTCAGAGCCGCGTTAACAGAAGTTGCTGGATCTGACTTTGGAATGCATGATGTAAGATGGT |
| | TATCTCGTTTGACCGATACAAGTAGACAAGCAGAAAGGTATAGAGATGGGAGAGTTCTTTTGGCTGGTGATGCAT |
| | GCCATATTCATTTGCCCGCTGGTGGCCAGGGACTGAACTTAGGATTTCAAGATGCCGTTAACTTGGGTTGGAAGC |
| | TTGGTGCTACCATTGCCGGGACCGCTCCACCAGAGTTATTGGATACGTATGAAGCTGAGAGAAGGCCGATAGCCG |
| | CCGGTGTTTTGAGAAATACAAGGGCTCAAGCTGTTCTAATTGATCCAGATCCTCGCTACGAGGGTTTAAGGGAAT |
| | TAATGATTGAATTGTTGCACGTTCCTGAGACTAATAGATATTTAGCGGGGCTAATCTCCGCATTAGACGTTAGAT |
| | ACCCAATGGCTGGGGAACATCCATTGCTGGGAAGAAGAGTACCCGACTTACCTCTTGTCACCGAAGATGGAACAA |
| | GACAGTTGTCCACTTATTTCCATGCTGCACGTGGCGTATTATTAACGTTAGGTTGTGATCAACCACTAGCAGATG |
| | AAGCCGCTGCTTGGAAAGATAGGGTTGATTTAGTTGCAGCTGAAGGTGTGGCGGACCCTGGTTCTGCAGTAGATG |
| | GCTTAACTGCTTTACTTGTAAGGCCTGATGGTTACATTTGTTGGACAGCTGCCCCAGAAACTGGTACTGATGGAT |
| | TGACAGACGCGCTGCGTACTTGGTTCGGCCCACCTGCAATGgtatcgatggattacaaggatgacgacgataaga |
| | tctgagctcttaattaacaattcttcgccagaggtttggtcaagtctccaatcaaggttgtcggct |
| | (SEQ ID NO: 67). The capitalized sequence is SEQ ID NO: 198. |
| FGD1-in- | ccctcactaaagggaacaaaagctggagctcAGTTTATCATTATCAATACTGCCATTTCAAAGAATACGTAAATA |
| pRS413-pGPD- | ATTAATAGTAGTGATTTTCCTAACTTTATTTAGTCAAAAAATTAGCCTTTTAATTCTGCTGTAACCCGTACATGC |
| ICYC1-AL-1- | CCAAAATAGGGGGCGGGTTACACAGAATATATAACATCGTAGGTGTCTGGGTGAACAGTTTATTCCTGGCATCCA |
| 215-D-C1 | CTAAATATAATGGAGCCCGCTTTTTAAGCTGGCATCCAGAAAAAAAAAGAATCCCAGCACCAAAATATTGTTTTC |
| | TTCACCAACCATCAGTTCATAGGTCCATTCTCTTAGCGCAACTACAGAGAACAGGGGCACAAACAGGCAAAAAAC |
| | GGGCACAACCTCAATGGAGTGATGCAACCTGCCTGGAGTAAATGATGACACAAGGCAATTGACCCACGCATGTAT |
| | CTATCTCATTTTCTTACACCTTCTATTACCTTCTGCTCTCTCTGATTTGGAAAAAGCTGAAAAAAAAGGTTGAAA |
| | CCAGTTCCCTGAAATTATTCCCCTACTTGACTAATAAGTATATAAAGACGGTAGGTATTGATTGTAATTCTGTAA |
| | ATCTATTTCTTAAACTTCTTAAATTCTACTTTTATAGTTAGTCTTTTTTTTAGTTTTAAAACACCAAGAACTTAG |
| | TTTCGACGGATACTAGTAAAATGGCTGAGTTGAAACTTGGCTACAAGGCATCAGCTGAACAGTTTGCCCCAAGGG |
| | AGTTAGTCGAACTAGCAGTCGCTGCCGAAGCTCACGGTATGGATTCCGCTACTGTTTCCGACCATTTCCAACCAT |
| | GGAGACACCAAGGTGGCCATGCACCATTCTCACTCAGTTGGATGACAGCTGTTGGAGAAAGAACTAATAGATTGT |
| | TATTGGGGACGTCGGTACTCACCCCGACGTTTAGATACAACCCTGCGGTAATAGCACAGGCCTTTGCTACAATGG |
| | GATGTCTATATCCAAACCGGGTGTTTTTAGGTGTTGGTACTGGAGAGGCCTTGAATGAAATCGCCACTGGTTATG |
| | AAGGTGCTTGGCCTGAGTTCAAAGAAAGGTTTGCGCGTCTGCGCGAAAGCGTGGGGCTGATGAGACAATTATGGT |
| | CTGGTGATAGGGTAGATTTTGATGGAGATTATTATAGATTAAAAGGCGCGTCTATATATGACGTTCCGGATGGTG |
| | GTGTCCCTGTATATATTGCCGCCGGAGGACCAGCAGTTGCTAAATATGCTGGCCGAGCTGGTGACGGTTTCATAT |
| | GCACTTCAGGCAAGGGTGAAGAACTTTACACAGAAAAGTTGATGCCCGCCGTCAGAGAAGGGGCGGCAGCCGCTG |
| | ACAGGTCTGTTGATGGCATAGACAAAATGATTGAGATCAAGATTTCATACGACCCAGATCCCGAATTAGCAATGA |
| | ATAACACAAGATTTTGGGCACCTCTTAGTTTGACCGCAGAACAAAAGCATAGCATCGATGATCCAATTGAAATGG |
| | AAAAAGCTGCTGATGCTTTACCCATCGAGCAAATTGCAAAAAGATGGATTGTTGCAAGTGATCCTGATGAAGCAG |
| | TGGAGAAAGTAGGACAGTACGTGACCTGGGGTTTAAATCATCTAGTTTTCCACGCTCCTGGGCATGATCAAAGAA |
| | GATTCTTGGAATTGTTTCAATCTGACCTAGCGCCAAGACTTCGTCGTCTGGGTTAACTCGAGtcatgtaattagt |
| | tatgtcacgcttacattcacgccctccccccacatccgctctaaccgaaaaggaaggagttagacaacctgaagt |
| | ctaggtccctatttatttttttatagttatgttagtattaagaacgttatttatatttcaaatttttcttttttt |
| | tctgtacagacgcgtgtacgcatgtaacattatactgaaaaccttgcttgagaaggttttgggacgctcgaaggc |
| | tttaatttgcggccggtacccaat (SEQ ID NO: 199). The capitalized sequence is |
| | SEQ ID NO: 200. |
| FNO-A- | ccctcactaaagggaacaaaagctggagctcAGTTTATCATTATCAATACTGCCATTTCAAAGAATACGTAAATA |
| fulgidus-in- | ATTAATAGTAGTGATTTTCCTAACTTTATTTAGTCAAAAAATTAGCCTTTTAATTCTGCTGTAACCCGTACATGC |
| pRS413-pGPD- | CCAAAATAGGGGGCGGGTTACACAGAATATATAACATCGTAGGTGTCTGGGTGAACAGTTTATTCCTGGCATCCA |
| tCYC1-AL-1- | CTAAATATAATGGAGCCCGCTTTTTAAGCTGGCATCCAGAAAAAAAAAGAATCCCAGCACCAAAATATTGTTTTC |
| 255-C1 | TTCACCAACCATCAGTTCATAGGTCCATTCTCTTAGCGCAACTACAGAGAACAGGGGCACAAACAGGCAAAAAAC |
| | GGGCACAACCTCAATGGAGTGATGCAACCTGCCTGGAGTAAATGATGACACAAGGCAATTGACCCACGCATGTAT |
| | CTATCTCATTTTCTTACACCTTCTATTACCTTCTGCTCTCTCTGATTTGGAAAAAGCTGAAAAAAAAGGTTGAAA |
| | CCAGTTCCCTGAAATTATTCCCCTACTTGACTAATAAGTATATAAAGACGGTAGGTATTGATTGTAATTCTGTAA |
| | ATCTATTTCTTAAACTTCTTAAATTCTACTTTTATAGTTAGTCTTTTTTTTAGTTTTAAAACACCAAGAACTTAG |
| | TTTCGACGGATACTAGTAAAATGAGGGTAGCTTTACTTGGTGGTACAGGAAATCTTGGAAAAGGCCTTGCCCTAC |
| | GACTGGCCACGCTGGGCCATGAAATCGTAGTCGGAAGCAGAAGAGAAGAAAAGGCAGAGGCTAAAGCAGCAGAAT |
| | ATAGGCGCATTGCAGGTGACGCTTCCATCACTGGTATGAAAAATGAAGATGCCGCTGAGGCATGCGACATTGCTG |
| | TCTTGACCATTCCTTGGGAACATGCTATTGACACTGCCAGAGATTTGAAGAATATTTTACGTGAGAAAATTGTTG |
| | TATCACCATTAGTTCCAGTGTCAAGAGGTGCAAAGGGCTTCACCTACTCGTCCGAAAGATCAGCGGCCGAGATTG |
| | TGGCTGAAGTTCTGGAATCTGAAAAAGTTGTTTCTGCGTTGCACACAATACCTGCTGCAAGATTTGCCAACTTGG |
| | ATGAAAAATTTGATTGGGATGTTCCTGTTTGTGGTGATGATGACGAAAGTAAGAAAGTCGTCATGTCTTTAATAA |
| | GTGAAATAGATGGGTTGAGGCCGTTAGATGCTGGTCCCCTCTCTAACTCCCGTTTAGTGGAGAGCCTAACTCCAT |
| | TGATATTGAACATCATGAGATTCAATGGAATGGGTGAACTAGGGATCAAGTTTTTATAACTCGAGtcatgtaatt |
| | agttatgtcacgcttacattcacgccctccccccacatccgctctaaccgaaaaggaaggagttagacaacctga |
| | agtctaggtccctatttatttttttatagttatgttagtattaagaacgttatttatatttcaaatttttctttt |
| | ttttctgtacagacgcgtgtacgcatgtaacattatactgaaaaccttgcttgagaaggttttgggacgctcgaa |
| | ggctttaatttgcggccggtacccaat (SEQ ID NO: 201). The capitalized sequence is |
| | SEQ ID NO: 202. |
| FNO-S-Griseus- | ccctcactaaagggaacaaaagctggagctcAGTTTATCATTATCAATACTGCCATTTCAAAGAATACGTAAATA |
| in-pRS413- | ATTAATAGTAGTGATTTTCCTAACTTTATTTAGTCAAAAAATTAGCCTTTTAATTCTGCTGTAACCCGTACATGC |
| pGPD-tCYC1- | CCAAAATAGGGGGCGGGTTACACAGAATATATAACATCGTAGGTGTCTGGGTGAACAGTTTATTCCTGGCATCCA |
| AL-1-235-C5 | CTAAATATAATGGAGCCCGCTTTTTAAGCTGGCATCCAGAAAAAAAAAGAATCCCAGCACCAAAATATTGTTTTC |
| | TTCACCAACCATCAGTTCATAGGTCCATTCTCTTAGCGCAACTACAGAGAACAGGGGCACAAACAGGCAAAAAAC |
| | GGGCACAACCTCAATGGAGTGATGCAACCTGCCTGGAGTAAATGATGACACAAGGCAATTGACCCACGCATGTAT |
| | CTATCTCATTTTCTTACACCTTCTATTACCTTCTGCTCTCTCTGATTTGGAAAAAGCTGAAAAAAAAGGTTGAAA |
| | CCAGTTCCCTGAAATTATTCCCCTACTTGACTAATAAGTATATAAAGACGGTAGGTATTGATTGTAATTCTGTAA |
| | ATCTATTTCTTAAACTTCTTAAATTCTACTTTTATAGTTAGTCTTTTTTTTAGTTTTAAAACACCAAGAACTTAG |
| | TTTCGACGGATACTAGTAAAATGACTACTCAAGACAGTGGGTCTGCACCGAAGCCTCCCGCCAAAGATCCATGGG |
| | ATTTGCCAGATGTCAGCGCACTGTCTGTTGGTGTCCTAGGTGGTACAGGACCACAGGGCAGGGGATTAGCCTACA |
| | GATTGGCGCGTGCTGGTCAAAAAGTGACCTTGGGCTCAAGAGATGCTGGACGCGCTGCTGATGCAGCGGCAGAGC |
| | TGGGCCATGGTGTGGAAGGTACTGATAATGCAGAATGCGCAAGAAGATCCGACATCGTTATTGTTGCTGTACCTT |
| | GGGACGGTCATGCTAAAACTTTGGAAAGTTTAAGAGAAGAGTTGGCTGGCAAGCTGGTTATCGACTGTGTTAACC |
| | CCTTGGGGTTTGATAAGAAAGGTGCTTATGCTTTAAAACCAGAGGAGGGCTCGGCCGCTGAACAAGCTGCGGCGC |
| | TTCTCCCTGATTCACGAGTAACAGCAGCTTTCCACCATTTATCTGCTGTGCTTTTACAAGATGAATCCATTGAAG |
| | AAATTGATACCGATGTTTTGGTCTTAGGTGAAGCAAGGGCAGACACGGATATTGTCCAGGCACTAGCAGGGCGTA |
| | TACCAGGTATGAGAGGAATATTTGCCGGTCGTCTAAGAGGTGCCCACCAAGTTGAATCACTTGTAGCCAATTTAA |
| | TATCTGTAAACAGAAGGTATAAGGCTCATGCCGGACTAAGGGCCACAGACGTGTAACTCGAGtcatgtaattagt |
| | tatgtcacgcttacattcacgccctccccccacatccgctctaaccgaaaaggaaggagttagacaacctgaagt |
| | ctaggtccctatttatttttttatagttatgttagtattaagaacgttatttatatttcaaatttttcttttttt |
| | tctgtacagacgcgtgtacgcatgtaacattatactgaaaaccttgcttgagaaggttttgggacgctcgaaggc |
| | tttaatttgcggccggtacccaat (SEQ ID NO: 203). The capitalized sequence is |
| | SEQ ID NO: 204. |
| pPGK1-oxyR- | Ccaagggggtggtttagtttagtagaacctcgtgaaacttacatttacatatatataaacttgcataaattggtc |
| tCYC1 in pSP- | aatgcaagaaatacatatttggtcttttctaattcgtagtttttcaagttcttagatgctttctttttctctttt |
| G1 AL-1-119- | ttacagatcatcaaggaagtaattatctactttttacaacaaatataaaacaaggatccATGCCATTCACTCAAA |
| A-C7 and AL-1- | AGGAGATCACGTATTTAAGGGCTCAAGGCTACGGCCGACTAGCCACCGTCGGTGCTCACGGTGAACCTCACAACG |
| 26-1-C1 | TGCCGGTATCTTTTGAAATTGATGAAGAAAGAGGTACCATTGAAATAACAGGAAGGGATATGGGACGTTCCAGAA |
| | AATTCAGAAATGTTGCCAAAAGTGACAGAGTGGCATTTGTTGTTGATGATGTTCCATGCAGAGACCCAGAAGTTG |
| | TCAGAGCTGTTGTAATACATGGTACTGCACAGGCGCTTCCCACAGGCGGAAGAGAAAGGCGTCCTCATTGTGCTG |
| | ACGAAATGATTAGAATTCATCCAAGAAGAATAGTAACATGGGGTATCGAAGGTGATTTGTCAACTGGTGTTCATG |
| | CAAGAGATATTACTGCTGAAGATGGTGGTAGAAGGgtcgacatggaacagaagttgatttccgaagaagacctcg |
| | agtaagcttggtaccgcggctagctaagatccgctctaaccgaaaaggaaggagttagacaacctgaagtctagg |
| | tccctatttatttttttatagttatgttagtattaagaacgttatttatatttcaaatttttcttttttttc |
| | (SEQ ID NO: 205). The capitalized sequence is SEQ ID NO: 206. |
| pPGK1-ctcM- | Ccaagggggtggtttagtttagtagaacctcgtgaaacttacatttacatatatataaacttgcataaattggtc |
| tCYC1 in pSP- | aatgcaagaaatacatatttggtcttttctaattcgtagtttttcaagttcttagatgctttctttttctctttt |
| G1 LR-23-C-C7 | ttacagatcatcaaggaagtaattatctactttttacaacaaatataaaacaaggatccATGCCATTTACAGCAA |
| | AAGAGGCTGCTTATTTGGCCTCTCAACCACTAGCCAGGTTGGCTACGGTGGATGGTCGTGGCCAGCCTCACGTCG |
| | TGCCGCTAGGGTTCCATTATAACGCAGAATTGGGCACCGTCGATGTTACTGGTAGAGGTATGGCTAGATCCTTAA |
| | AGTACAGACATGTTCAAGGTCATCCAAGAGTATCATTAGTAGTTGACGATATTGTAGACGCTCAAAGATGGGTTG |
| | TCCGTGGAATAGAGATCAGGGGTACTGCTGTTGCACTTGCCACCGGAGGAAAAGAAATTCTGCCACACGTAGATG |
| | ATGAATTAATACGAATCACACCTGAAAGAATTGTTAGTTGGGGTATTGACAGCGATTGTCAAGCCCCCCCTCTCG |
| | CGAGGAATGTGGGGGCGGGCGCACAGGCAgtcgacatggaacagaagttgatttccgaagaagacctcgagtaag |
| | cttggtaccgcggctagctaagatccgctctaaccgaaaaggaaggagttagacaacctgaagtctaggtcccta |
| | tttatttttttatagttatgttagtattaagaacgttatttatatttcaaatttttcttttttttc |
| | (SEQ ID NO: 207). The capitalized sequence is SEQ ID NO: 208. |
| pPGK1-dac04- | Ccaagggggtggtttagtttagtagaacctcgtgaaacttacatttacatatatataaacttgcataaattggtc |
| tCYC1 in pSP- | aatgcaagaaatacatatttggtcttttctaattcgtagtttttcaagttcttagatgctttctttttctctttt |
| G1 LR-23-A-C3 | ttacagatcatcaaggaagtaattatctactttttacaacaaatataaaacaaggatccATGTCATTCACTGCCA |
| | AGGAAGTTCAATATTTGCGTTCTCAGCAATTAGGAAGATTAGCCACCGTTGGTGCTGATGGTACACCACATAATG |
| | TTCCAGTAGGCTACAGATACAACGCTGACTTGGGGACCATTGACATCACAGGAAGGGACTTAAGAAGAAGTAGGA |
| | AATATAGAGATGTTCAGGCTGGCTCGCGGGTGGCATTTATTGTTGATGATCTACCGAGCACAGCACCTATAGTCG |
| | CCCGCGGGCTGGAGGTGAGGGGAACAGCTGAGGCGCTTTCTGGTGAAGAAGATTTGATACGTGTACGACCTGCAA |
| | GAATCGTCACTTGGGGTATTGAAGCAGATTGGCAAGCTGGTCCTACTGGTAGAACGGTAAGGCCCACCACTCCAA |
| | GATCCGCGACGgtcgacatggaacagaagttgatttccgaagaagacctcgagtaagcttggtaccgcggctagc |
| | taagatccgctctaaccgaaaaggaaggagttagacaacctgaagtctaggtccctatttatttttttatagtta |
| | tgttagtattaagaacgttatttatatttcaaatttttcttttttttc (SEQ ID NO: 209). |
| | The capitalized sequence is SEQ ID NO: 146. |

### Example 13. Mutagenesis Screen of OxyS

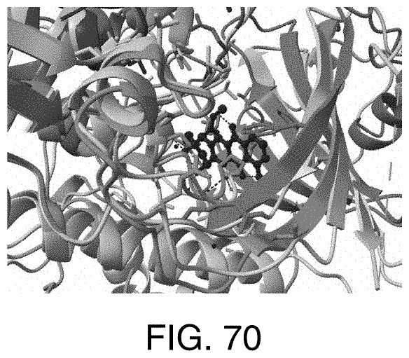

Native OxyS shows no detectable activity with TAN-1612 as a substrate. This Example provides the results of a mutagenesis screen of OxyS to accept TAN-1612 as a substrate. The binding pocket of OxyS was identified by analysis of the structure of a homologous protein, Aklavinone-11-Hydroxylase, along with its native substrate, aklavinone (PDB ID: 4K2X and 3IHG, respectively) (FIG. 70) Amino acid residues <6 Å away from the binding pocket were mutated through saturation mutagenesis. A total of 24 sites were mutated (K42X, A43X, L44X, G45X, L95X, F96X, M176X, W211X, F212X, T225X, A227X, F228X, V240X, P295X, A296X, G297X, G298X, G299X, N302X, I353X, D354X, R358X, V372X and P375X) for a library size of 480. The nucleotide and amino acid sequences of wild-type OxyS and OxyS with a L44F, G45A or G299L mutation are shown in Tables 36-40.

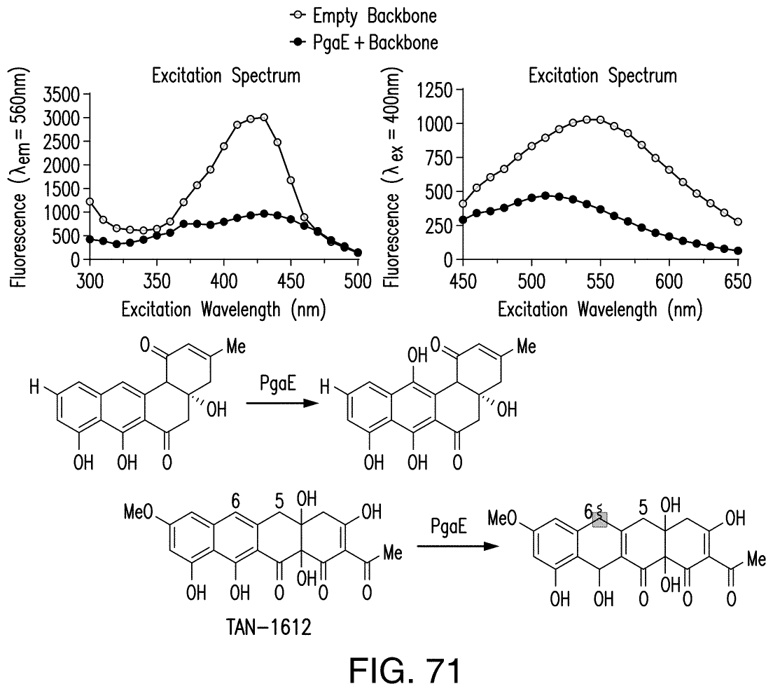

As a positive control for the mutagenesis screen, the hydroxylase PgaE, which shows some activity for TAN-1612, was used. FIG. 71 provides the reaction of PgaE with its natural substrate and the hypothesized reaction of PgaE with TAN-1612. As shown in FIG. 72, a mass at 445.0776 m/z was identified by mass spectrometry in the reaction that included PgaE as compared to control. This mass appears to correspond to a doubly hydroxylated TAN-1612 molecule, i.e., hydroxyl groups at the 5 and 6 position. The reactions in the presence of yeast expressing an OxyS L44F, G45A or Q299L mutant also showed intense peaks that correspond with this mass (FIG. 72).

After mass spectrometry analysis, 10 mutations proved to have the most intense peaks correlating to the 445.0776 m/z mass (Table 35.). The DNA of these strains were purified and transformed into E. coli to confirm their mutations, shown below.

TABLE 35
Confirmed Mutations

| | | |
|---|---|---|
| A43C | G45A | Q299S |
| L44T | F228L | Q299L |
| L44F | F228R | P357R |

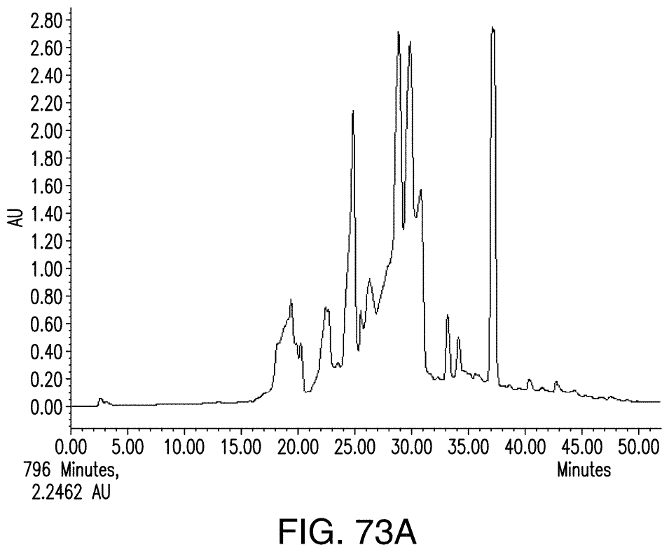

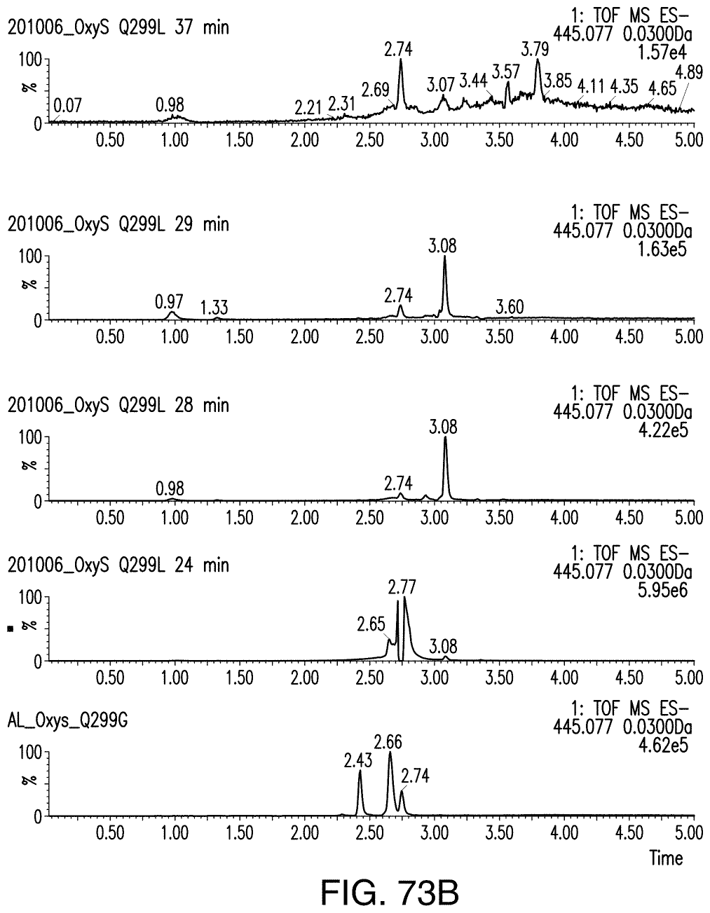

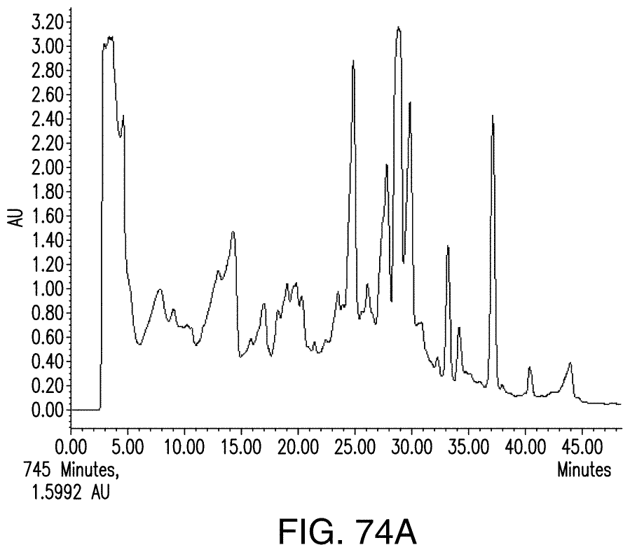

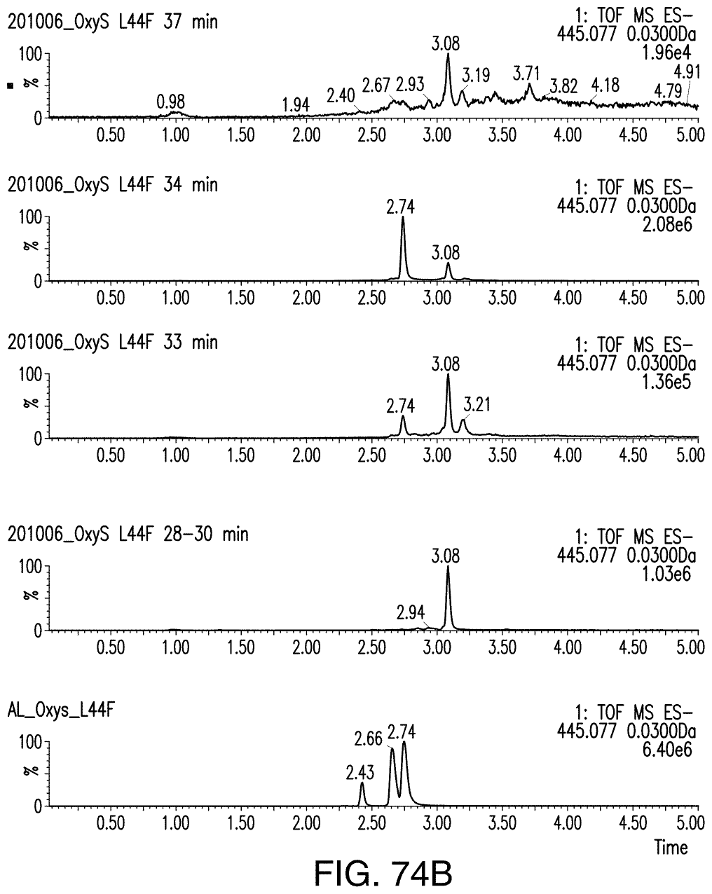

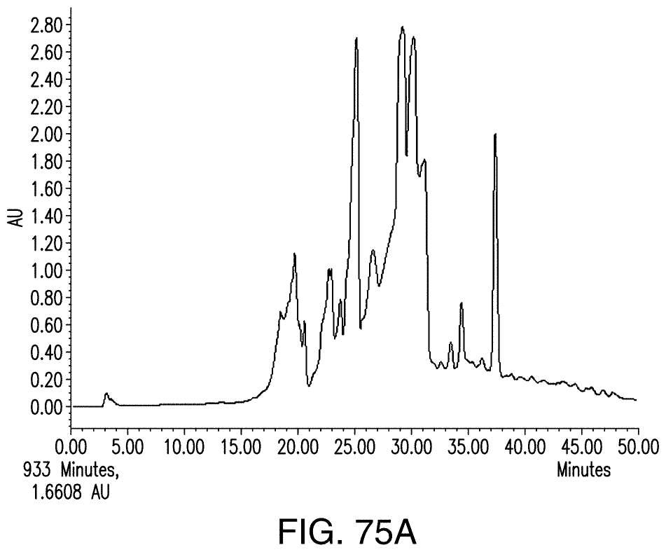

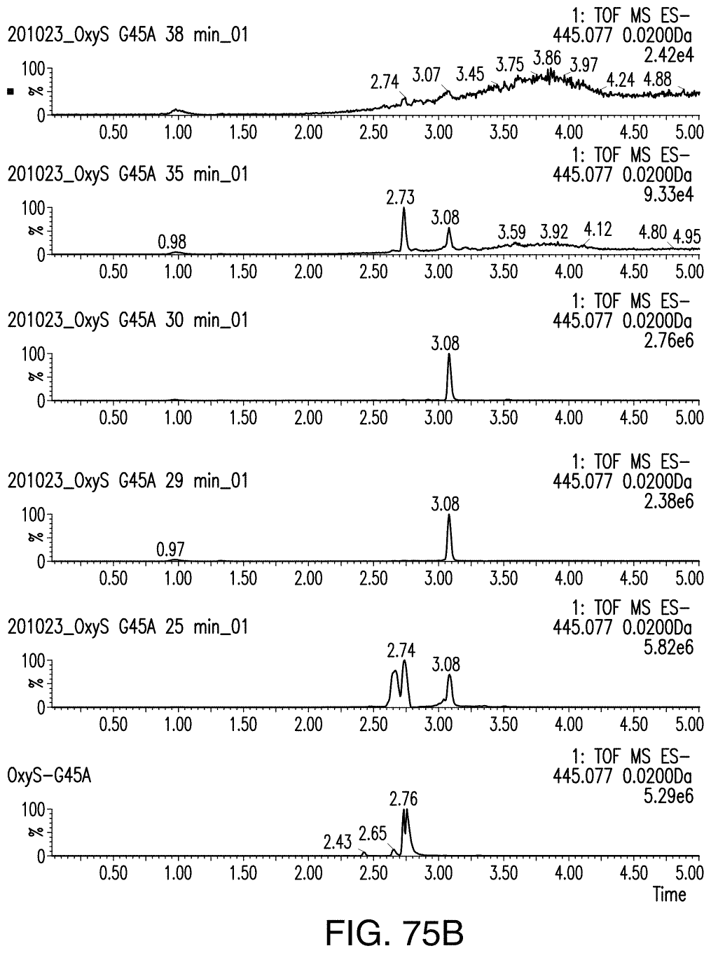

HPLC and mass spectrometry was performed to determine which OxyS mutants resulted in the hydroxylation of TAN-1612. HPLC was performed to isolate fractions of the reaction catalyzed by OxyS Q299L (FIG. 73A). Fractions at 24, 28, 29, and 37 minutes were collected and then analyzed by mass spectrometry (FIG. 73B). As shown in FIG. 73B, no fraction corresponded to either the 2.66 or 2.74 min elution time that would indicate the doubly hydroxylated molecule. HPLC was performed to isolate fractions of the reaction catalyzed by OxyS L44F (FIG. 74A), and fractions at 28-30, 33, 34 and 37 minutes were collected and analyzed by mass spectrometry (FIG. 74B). As shown in FIG. 74B, the purified fraction at 34 min corresponds to the 2.74 min elution time, which is the hypothesized doubly hydroxylated TAN-1612. HPLC was performed to isolate fractions of the reaction catalyzed by OxyS G45A (FIG. 75A), and fractions at 25, 29, 30, 35 and 38 minutes were collected and analyzed by mass spectrometry (FIG. 75B). As shown in FIG. 75B, the purified fraction at 35 min corresponds to the 2.74 min elution time, which is the hypothesized doubly hydroxylated TAN-1612.

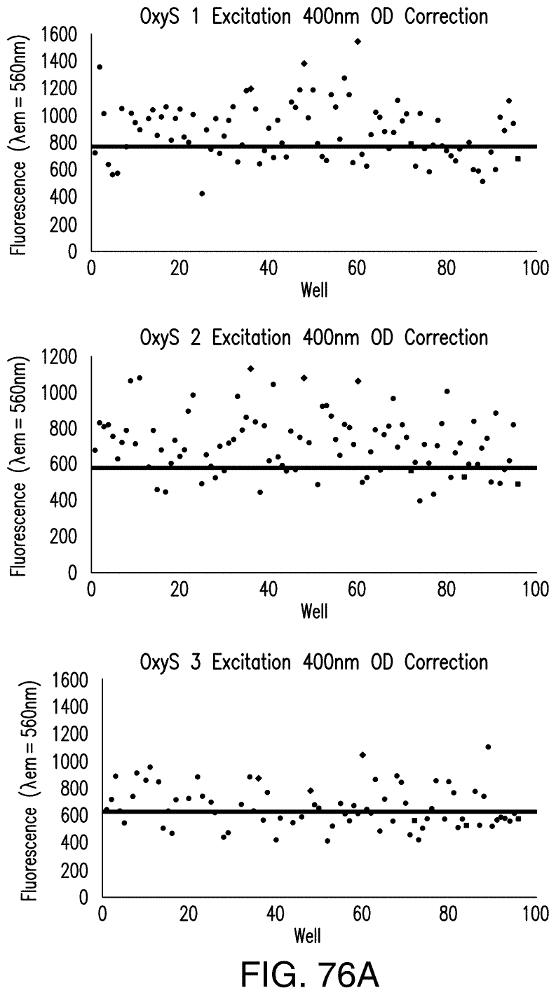

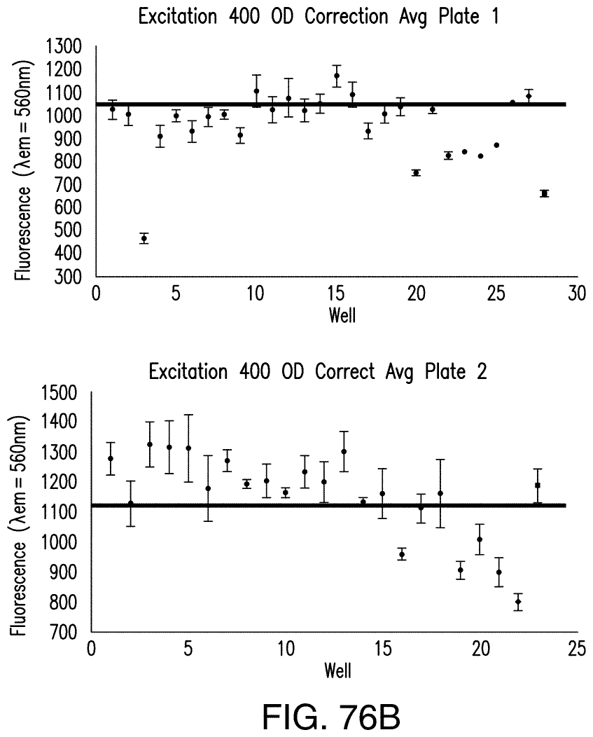

In FIG. 76A, which is based on FIG. 71, data was taken at the 400 nm excitation, and a 560 nm emission spectrum indicates a lower fluorescence with PgaE ( [symbol] ) and the positive control compared to the empty backbone pSP-G1 ( [symbol] ). Colonies that had an equal or lower fluorescence than PgaE were selected as potential hits for further analysis (56 out of 264). OxyS 1, 2, and 3 correspond to 3 separate 96-well plates. FIG. 76B provides a repeat of the UV/Vis assay of FIG. 76A with PgaE ( [symbol] ) and empty backbone pSP-G1 ( [symbol] ), where each sample was repeated 6×. This repeated screen led to 25 potential hits to move forward with and to analyze with mass spectrometry.

TABLE 36
Nucleotide Sequence of OxyS-L44F in the
pSP-Gl plasmid in FY251 strain.

Sequence for OxyS from Streptomyces rimosus
with an L44F mutation is shown capitalized
within the context of the pSP-G1 backbone
containing pTEF1, the FLAG tag and tADH1
(Addgene 64736) (partial, decapitalized).
tcgcgcgtttcggtgatgacggtgaaaacctctgacacatgcagctcc
cggagacggtcacagcttgtctgtaagcggatgccgggagcagacaag
cccgtcagggcgcgtcagcgggtgttggcgggtgtcggggctggctta
actatgcggcatcagagcagattgtactgagagtgcaccataccacag
cttttcaattcaattcatcattttttttttattcttttttttgatttc
ggtttctttgaaatttttttgattcggtaatctccgaacagaaggaag
aacgaaggaaggagcacagacttagattggtatatatacgcatatgta
gtgttgaagaaacatgaaattgcccagtattcttaacccaactgcaca
gaacaaaaacctgcaggaaacgaagataaatcatgtcgaaagctacat
ataaggaacgtgctgctactcatcctagtcctgttgctgccaagctat
ttaatatcatgcacgaaaagcaaacaaacttgtgtgcttcattggatg
ttcgtaccaccaaggaattactggagttagttgaagcattaggtccca
aaatttgtttactaaaaacacatgtggatatcttgactgatttttcca
tggagggcacagttaagccgctaaaggcattatccgccaagtacaatt
ttttactcttcgaagacagaaaatttgctgacattggtaatacagtca
aattgcagtactctgcgggtgtatacagaatagcagaatgggcagaca
ttacgaatgcacacggtgtggtgggcccaggtattgttagcggtttga
agcaggcggcagaagaagtaacaaaggaacctagaggccttttgatgt
tagcagaattgtcatgcaagggctccctatctactggagaatatacta
agggtactgttgacattgcgaagagcgacaaagattttgttatcggct
ttattgctcaaagagacatgggtggaagagatgaaggttacgattggt
tgattatgacacccggtgtgggtttagatgacaagggagacgcattgg
gtcaacagtatagaaccgtggatgatgtggtctctacaggatctgaca
ttattattgttggaagaggactatttgcaaagggaagggatgctaagg
tagagggtgaacgttacagaaaagcaggctgggaagcatatttgagaa
gatgcggccagcaaaactaaaaaactgtattataagtaaatgcatgta
tactaaactcacaaattagagcttcaatttaattatatcagttattac
cctatgcggtgtgaaataccgcacagatgcgtaaggagaaaataccgc
atcaggaaattgtaaacgttaatattttgttaaaattcgcgttaaatt
tttgttaaatcagctcattttttaaccaataggccgaaatcggcaaaa
tcccttataaatcaaaagaatagaccgagatagggttgagtgttgttc
cagtttggaacaagagtccactattaaagaacgtggactccaacgtca
aagggcgaaaaaccgtctatcagggcgatggcccactacgtgaaccat
caccctaatcaagttttttggggtcgaggtgccgtaaagcactaaatc
ggaaccctaaagggagcccccgatttagagcttgacggggaaagccgg
cgaacgtggcgagaaaggaagggaagaaagcgaaaggagcgggcgcta
gggcgctggcaagtgtagcggtcacgctgcgcgtaaccaccacacccg
ccgcgcttaatgcgccgctacagggcgcgtccattcgccattcaggct
gcgcaactgttgggaagggcgatcggtgcgggcctcttcgctattacg
ccagctgaattggagcgacctcatgctatacctgagaaagcaacctga
cctacaggaaagagttactcaagaataagaattttcgttttaaaacct
aagagtcactttaaaatttgtatacacttattttttttataacttatt
taataataaaaatcataaatcataagaaattcgcttatttagaagtgt
caacaacgtatctaccaacgatttgacccttttccatcttttcgtaaa
tttctggcaaggtagacaagccgacaaccttgattggagacttgacca
aacctctggcgaagaattgttaattaagagctcagatcttatcgtcgt
catccttgtaatccatcgatacCATTGCAGGTGGGCCGAACCAAGTAC
GCAGCGCGTCTGTCAATCCATCAGTACCAGTTTCTGGGGCAGCTGTCC
AACAAATGTAACCATCAGGCCTTACAAGTAAAGCAGTTAAGCCATCTA
CTGCAGAACCAGGGTCCGCCACACCTTCAGCTGCAACTAAATCAACCC
TATCTTTCCAAGCAGCGGCTTCATCTGCTAGTGGTTGATCACAACCTA
ACGTTAATAATACGCCACGTGCAGCATGGAAATAAGTGGACAACTGTC
TTGTTCCATCTTCGGTGACAAGAGGTAAGTCGGGTACTCTTCTTCCCA
GCAATGGATGTTCCCCAGCCATTGGGTATCTAACGTCTAATGCGGAGA
TTAGCCCCGCTAAATATCTATTAGTCTCAGGAACGTGCAACAATTCAA
TCATTAATTCCCTTAAACCCTCGTAGCGAGGATCTGGATCAATTAGAA
CAGCTTGAGCCCTTGTATTTCTCAAAACACCGGCGGCTATCGGCCTTC
TCTCAGCTTCATACGTATCCAATAACTCTGGTGGAGCGGTCCCGGCAA
TGGTAGCACCAAGCTTCCAACCCAAGTTAACGGCATCTTGAAATCCTA
AGTTCAGTCCCTGGCCACCAGCGGGCAAATGAATATGGCATGCATCAC
CAGCCAAAAGAACTCTCCCATCTCTATACCTTTCTGCTTGTCTACTTG
TATCGGTCAAACGAGATAACCATCTTACATCATGCATTCCAAAGTCAG
ATCCAGCAACTTCTGTTAACGCGGCTCTGACATCCTCCTCCGTTACCG
GCGCCCTCCTGTCAGCATACGGTCTATCGAAAAAGGCGACTGTTGCTC
TGTAGACGTCTGGTTCTAGCGGAAATGCTGCGAACCATGCACGAAGGT
CATGTCTCATGACACCATAAGGACGCATAGGACCCATACCTTCTCCGT
GAGGAAGCTCTTCTCTGAATCTTGCGTCTGCGATGACAGCAAACATAT
GTGGGTCTTGACCTGGAAAATCTATGCCCAATAGTTTCCTCACCGTAC
TGCCTCCACCGTCGCAGCCCACCAAATAAGCACATTCTACTTCGTATG
GGCCTTCAGGGCCTGTCACGCTCACAGTAACACCATCAGCATCTTGTT
CCAAGGCGGTCACGGCATGACCACGACGCAGCTCCGCCCCTAGCTCCA
AAGCTCTACCTGTTAGCAGTTCTTCAGTTCGTACTTGTGGAACAAACA
ATGCATATGGGTGTCTAGTATCAAATGATGAGAAATCCAGGGGGACGC
CTAATGAGGCAAAATTACCACCTCTTAACTTTGGTGCTTCAGCCTGGA
ATCCTTCACCGAGGCCTCTCATATCTAATAGTTCAACAGTGCGAGCGT
GGACTCCAAAAGCTTTCGAGAAGTCAACAGGCTCGGCTAATCTTTCTA
AAACCAAAGTTCTGGCACCCGCCAGCCGAAGTTCACATGCTAACATCA
AACCGGTGGGTCCTGCACCAGCTATAACAACATCGTACCTCATgcggc
cgcttgtaattaaaacttagattagattgctatgctttctttctaatg
agcaagaagtaaaaaaagttgtaatagaacaagaaaaatgaaactgaa
acttgagaaattgaagaccgtttattaacttaaatatcaatgggaggt
catcgaaagagaaaaaaatcaaaaaaaaaaattttcaagaaaaagaaa
cgtgataaaaatttttattgcctttttcgacgaagaaaaagaaacgag
gcggtctcttttttcttttccaaacctttagtacgggtaattaacgac
accctagaggaagaaagaggggaaatttagtatgctgtgcttgggtgt
tttgaagtggtacggcgatgcgcggagtccgagaaaatctggaagagt
aaaaaaggagtagaaacattttgaagctatggtgtgtgcggccggcct
ggaagtaccttcaaagaatggggtcttatcttgttttgcaagtaccac
tgagcaggataataatagaaatgataatatactatagtagagataacg
tcgatgacttcccatactgtaattgcttttagttgtgtatttttagtg
tgcaagtttctgtaaatcgattaatttttttttctttcctctttttat
taaccttaatttttattttagattcctgacttcaactcaagacgcaca
gatattataacatctgcataataggcatttgcaagaattactcgtgag
taaggaaagagtgaggaactatcgcatacctgcatttaaagatgccga
tttgggcgcgaatcctttattttggcttcaccctcatactattatcag
ggccagaaaaaggaagtgtttccctccttcttgaattgatgttaccct
cataaagcacgtggcctcttatcgagaaagaaattaccgtcgctcgtg
atttgtttgcaaaaagaacaaaactgaaaaaacccagacacgctcgac
ttcctgtcttcctattgattgcagcttccaatttcgtcacacaacaag
gtcctagcgacggctcacaggttttgtaacaagcaatcgaaggttctg
gaatggcgggaaagggtttagtaccacatgctatgatgcccactgtga
tctccagagcaaagttcgttcgatcgtactgttactctctctctttca
aacagaattgtccgaatcgtgtgacaacaacagcctgttctcacacac
tcttttcttctaaccaagggggtggtttagtttagtagaacctcgtga
aacttacatttacatatatataaacttgcataaattggtcaatgcaag
aaatacatatttggtcttttctaattcgtagtttttcaagttcttaga
tgctttctttttctcttttttacagatcatcaaggaagtaattatcta
ctttttacaacaaatataaaacaaggatccgtaatacgactcactata
gggcccgggcgtcgacatggaacagaagttgatttccgaagaagacct
cgagtaagcttggtaccgcggctagctaagatccgctctaaccgaaaa
ggaaggagttagacaacctgaagtctaggtccctatttatttttttat
agttatgttagtattaagaacgttatttatatttcaaatttttctttt
ttttctgtacagacgcgtgtacgcatgtaacattatactgaaaacctt
gcttgagaaggttttgggacgctcgaagatccagctgcattaatgaat
cggccaacgcgcggggagaggcggtttgcgtattgggcgctcttccgc
ttcctcgctcactgactcgctgcgctcggtcgttcggctgcggcgagc
ggtatcagctcactcaaaggcggtaatacggttatccacagaatcagg
ggataacgcaggaaagaacatgtgagcaaaaggccagcaaaaggccag
gaaccgtaaaaaggccgcgttgctggcgtttttccataggctccgccc
ccctgacgagcatcacaaaaatcgacgctcaagtcagaggtggcgaaa
cccgacaggactataaagataccaggcgtttccccctggaagctccct
cgtgcgctctcctgttccgaccctgccgcttaccggatacctgtccgc
ctttctcccttcgggaagcgtggcgctttctcatagctcacgctgtag
gtatctcagttcggtgtaggtcgttcgctccaagctgggctgtgtgca
cgaaccccccgttcagcccgaccgctgcgccttatccggtaactatcg
tcttgagtccaacccggtaagacacgacttatcgccactggcagcagc
cactggtaacaggattagcagagcgaggtatgtaggcggtgctacaga
gttcttgaagtggtggcctaactacggctacactagaaggacagtatt
tggtatctgcgctctgctgaagccagttaccttcggaaaaagagttgg
tagctcttgatccggcaaacaaaccaccgctggtagcggtggtttttt
tgtttgcaagcagcagattacgcgcagaaaaaaaggatctcaagaaga
tcctttgatcttttctacggggtctgacgctcagtggaacgaaaactc
acgttaagggattttggtcatgagattatcaaaaaggatcttcaccta
gatccttttaaattaaaaatgaagttttaaatcaatctaaagtatata
tgagtaaacttggtctgacagttaccaatgcttaatcagtgaggcacc
tatctcagcgatctgtctatttcgttcatccatagttgcctgactccc
cgtcgtgtagataactacgatacgggagggcttaccatctggccccag
tgctgcaatgataccgcgagacccacgctcaccggctccagatttatc
agcaataaaccagccagccggaagggccgagcgcagaagtggtcctgc
aactttatccgcctccatccagtctattaattgttgccgggaagctag
agtaagtagttcgccagttaatagtttgcgcaacgttgttgccattgc
tacaggcatcgtggtgtcacgctcgtcgtttggtatggcttcattcag
ctccggttcccaacgatcaaggcgagttacatgatcccccatgttgtg
caaaaaagcggttagctccttcggtcctccgatcgttgtcagaagtaa
gttggccgcagtgttatcactcatggttatggcagcactgcataattc
tcttactgtcatgccatccgtaagatgcttttctgtgactggtgagta
ctcaaccaagtcattctgagaatagtgtatgcggcgaccgagttgctc
ttgcccggcgtcaatacgggataataccgcgccacatagcagaacttt
aaaagtgctcatcattggaaaacgttcttcggggcgaaaactctcaag
gatcttaccgctgttgagatccagttcgatgtaacccactcgtgcacc
caactgatcttcagcatcttttactttcaccagcgtttctgggtgagc
aaaaacaggaaggcaaaatgccgcaaaaaagggaataagggcgacacg
gaaatgttgaatactcatactcttcctttttcaatattattgaagcat
ttatcagggttattgtctcatgagcggatacatatttgaatgtattta
gaaaaataaacaaataggggttccgcgcacatttccccgaaaagtgcc
acctgaacgaagcatctgtgcttcattttgtagaacaaaaatgcaacg
cgagagcgctaatttttcaaacaaagaatctgagctgcatttttacag
aacagaaatgcaacgcgaaagcgctattttaccaacgaagaatctgtg
cttcatttttgtaaaacaaaaatgcaacgcgagagcgctaatttttca
aacaaagaatctgagctgcatttttacagaacagaaatgcaacgcgag
agcgctattttaccaacaaagaatctatacttcttttttgttctacaa
aaatgcatcccgagagcgctatttttctaacaaagcatcttagattac
tttttttctcctttgtgcgctctataatgcagtctcttgataactttt
tgcactgtaggtccgttaaggttagaagaaggctactttggtgtctat
tttctcttccataaaaaaagcctgactccacttcccgcgtttactgat
tactagcgaagctgcgggtgcattttttcaagataaaggcatccccga
ttatattctataccgatgtggattgcgcatactttgtgaacagaaagt
gatagcgttgatgattcttcattggtcagaaaattatgaacggtttct
tctattttgtctctatatactacgtataggaaatgtttacattttcgt
attgttttcgattcactctatgaatagttcttactacaatttttttgt
ctaaagagtaatactagagataaacataaaaaatgtagaggtcgagtt
tagatgcaagttcaaggagcgaaaggtggatgggtaggttatataggg
atatagcacagagatatatagcaaagagatacttttgagcaatgtttg
tggaagcggtattcgcaatattttagtagctcgttacagtccggtgcg
tttttggttttttgaaagtgcgtcttcagagcgcttttggttttcaaa
agcgctctgaagttcctatactttctagagaataggaacttcggaata
ggaacttcaaagcgtttccgaaaacgagcgcttccgaaaatgcaacgc
gagctgcgcacatacagctcactgttcacgtcgcacctatatctgcgt
gttgcctgtatatatatatacatgagaagaacggcatagtgcgtgttt
atgcttaaatgcgtacttatatgcgtctatttatgtaggatgaaaggt
agtctagtacctcctgtgatattatcccattccatgcggggtatcgta
tgcttccttcagcactaccctttagctgttctatatgctgccactcct
caattggattagtctcatccttcaatgctatcatttcctttgatattg
gatcatactaagaaaccattattatcatgacattaacctataaaaata
ggcgtatcacgaggccctttcgtc (SEQ ID NO: 192). The
capitalized sequence is SEQ ID NO: 193.

TABLE 37
Nucleotide Sequence of OxyS-G45A in the
pSP-G1 plasmid in FY251 strain.

Sequence for OxyS from Streptomyces rimosus
with a G45A mutation is shown capitalized
within the context of the pSP-G1 backbone
containing pTEF1, the FLAG tag and tADH1
(Addgene 64736) (partial, decapitalized).
tcgcgcgtttcggtgatgacggtgaaaacctctgacacatgcagctcc
cggagacggtcacagcttgtctgtaagcggatgccgggagcagacaag
cccgtcagggcgcgtcagcgggtgttggcgggtgtcggggctggctta
actatgcggcatcagagcagattgtactgagagtgcaccataccacag
cttttcaattcaattcatcattttttttttattcttttttttgatttc
ggtttctttgaaatttttttgattcggtaatctccgaacagaaggaag
aacgaaggaaggagcacagacttagattggtatatatacgcatatgta
gtgttgaagaaacatgaaattgcccagtattcttaacccaactgcaca
gaacaaaaacctgcaggaaacgaagataaatcatgtcgaaagctacat
ataaggaacgtgctgctactcatcctagtcctgttgctgccaagctat
ttaatatcatgcacgaaaagcaaacaaacttgtgtgcttcattggatg
ttcgtaccaccaaggaattactggagttagttgaagcattaggtccca
aaatttgtttactaaaaacacatgtggatatcttgactgatttttcca
tggagggcacagttaagccgctaaaggcattatccgccaagtacaatt
ttttactcttcgaagacagaaaatttgctgacattggtaatacagtca
aattgcagtactctgcgggtgtatacagaatagcagaatgggcagaca
ttacgaatgcacacggtgtggtgggcccaggtattgttagcggtttga
agcaggcggcagaagaagtaacaaaggaacctagaggccttttgatgt
tagcagaattgtcatgcaagggctccctatctactggagaatatacta
agggtactgttgacattgcgaagagcgacaaagattttgttatcggct
ttattgctcaaagagacatgggtggaagagatgaaggttacgattggt
tgattatgacacccggtgtgggtttagatgacaagggagacgcattgg
gtcaacagtatagaaccgtggatgatgtggtctctacaggatctgaca
ttattattgttggaagaggactatttgcaaagggaagggatgctaagg
tagagggtgaacgttacagaaaagcaggctgggaagcatatttgagaa
gatgcggccagcaaaactaaaaaactgtattataagtaaatgcatgta
tactaaactcacaaattagagcttcaatttaattatatcagttattac
cctatgcggtgtgaaataccgcacagatgcgtaaggagaaaataccgc
atcaggaaattgtaaacgttaatattttgttaaaattcgcgttaaatt
tttgttaaatcagctcattttttaaccaataggccgaaatcggcaaaa
tcccttataaatcaaaagaatagaccgagatagggttgagtgttgttc
cagtttggaacaagagtccactattaaagaacgtggactccaacgtca
aagggcgaaaaaccgtctatcagggcgatggcccactacgtgaaccat
caccctaatcaagttttttggggtcgaggtgccgtaaagcactaaatc
ggaaccctaaagggagcccccgatttagagcttgacggggaaagccgg
cgaacgtggcgagaaaggaagggaagaaagcgaaaggagcgggcgcta
gggcgctggcaagtgtagcggtcacgctgcgcgtaaccaccacacccg
ccgcgcttaatgcgccgctacagggcgcgtccattcgccattcaggct
gcgcaactgttgggaagggcgatcggtgcgggcctcttcgctattacg
ccagctgaattggagcgacctcatgctatacctgagaaagcaacctga
cctacaggaaagagttactcaagaataagaattttcgttttaaaacct
aagagtcactttaaaatttgtatacacttattttttttataacttatt
taataataaaaatcataaatcataagaaattcgcttatttagaagtgt
caacaacgtatctaccaacgatttgacccttttccatcttttcgtaaa
tttctggcaaggtagacaagccgacaaccttgattggagacttgacca
aacctctggcgaagaattgttaattaagagctcagatcttatcgtcgt
catccttgtaatccatcgatacCATTGCAGGTGGGCCGAACCAAGTAC
GCAGCGCGTCTGTCAATCCATCAGTACCAGTTTCTGGGGCAGCTGTCC
AACAAATGTAACCATCAGGCCTTACAAGTAAAGCAGTTAAGCCATCTA
CTGCAGAACCAGGGTCCGCCACACCTTCAGCTGCAACTAAATCAACCC
TATCTTTCCAAGCAGCGGCTTCATCTGCTAGTGGTTGATCACAACCTA
ACGTTAATAATACGCCACGTGCAGCATGGAAATAAGTGGACAACTGTC
TTGTTCCATCTTCGGTGACAAGAGGTAAGTCGGGTACTCTTCTTCCCA
GCAATGGATGTTCCCCAGCCATTGGGTATCTAACGTCTAATGCGGAGA
TTAGCCCCGCTAAATATCTATTAGTCTCAGGAACGTGCAACAATTCAA
TCATTAATTCCCTTAAACCCTCGTAGCGAGGATCTGGATCAATTAGAA
CAGCTTGAGCCCTTGTATTTCTCAAAACACCGGCGGCTATCGGCCTTC
TCTCAGCTTCATACGTATCCAATAACTCTGGTGGAGCGGTCCCGGCAA
TGGTAGCACCAAGCTTCCAACCCAAGTTAACGGCATCTTGAAATCCTA
AGTTCAGTCCCTGGCCACCAGCGGGCAAATGAATATGGCATGCATCAC
CAGCCAAAAGAACTCTCCCATCTCTATACCTTTCTGCTTGTCTACTTG
TATCGGTCAAACGAGATAACCATCTTACATCATGCATTCCAAAGTCAG
ATCCAGCAACTTCTGTTAACGCGGCTCTGACATCCTCCTCCGTTACCG
GCGCCCTCCTGTCAGCATACGGTCTATCGAAAAAGGCGACTGTTGCTC
TGTAGACGTCTGGTTCTAGCGGAAATGCTGCGAACCATGCACGAAGGT
CATGTCTCATGACACCATAAGGACGCATAGGACCCATACCTTCTCCGT
GAGGAAGCTCTTCTCTGAATCTTGCGTCTGCGATGACAGCAAACATAT
GTGGGTCTTGACCTGGAAAATCTATGCCCAATAGTTTCCTCACCGTAC
TGCCTCCACCGTCGCAGCCCACCAAATAAGCACATTCTACTTCGTATG
GGCCTTCAGGGCCTGTCACGCTCACAGTAACACCATCAGCATCTTGTT
CCAAGGCGGTCACGGCATGACCACGACGCAGCTCCGCCCCTAGCTCCA
AAGCTCTACCTGTTAGCAGTTCTTCAGTTCGTACTTGTGGAACAAACA
ATGCATATGGGTGTCTAGTATCAAATGATGAGAAATCCAGGGGGACGC
CTAATGAGGCAAAATTACCACCTCTTAACTTTGGTGCTTCAGCCTGGA
ATCCTTCACCGAGGCCTCTCATATCTAATAGTTCAACAGTGCGAGCGT
GGACCGCTAGAGCTTTCGAGAAGTCAACAGGCTCGGCTAATCTTTCTA
AAACCAAAGTTCTGGCACCCGCCAGCCGAAGTTCACATGCTAACATCA
AACCGGTGGGTCCTGCACCAGCTATAACAACATCGTACCTCATgcggc
cgcttgtaattaaaacttagattagattgctatgctttctttctaatg
agcaagaagtaaaaaaagttgtaatagaacaagaaaaatgaaactgaa
acttgagaaattgaagaccgtttattaacttaaatatcaatgggaggt
catcgaaagagaaaaaaatcaaaaaaaaaaattttcaagaaaaagaaa
cgtgataaaaatttttattgcctttttcgacgaagaaaaagaaacgag
gcggtctcttttttcttttccaaacctttagtacgggtaattaacgac
accctagaggaagaaagaggggaaatttagtatgctgtgcttgggtgt
tttgaagtggtacggcgatgcgcggagtccgagaaaatctggaagagt
aaaaaaggagtagaaacattttgaagctatggtgtgtgcggccggcct
ggaagtaccttcaaagaatggggtcttatcttgttttgcaagtaccac
tgagcaggataataatagaaatgataatatactatagtagagataacg
tcgatgacttcccatactgtaattgcttttagttgtgtatttttagtg
tgcaagtttctgtaaatcgattaatttttttttctttcctctttttat
taaccttaatttttattttagattcctgacttcaactcaagacgcaca
gatattataacatctgcataataggcatttgcaagaattactcgtgag
taaggaaagagtgaggaactatcgcatacctgcatttaaagatgccga
tttgggcgcgaatcctttattttggcttcaccctcatactattatcag
ggccagaaaaaggaagtgtttccctccttcttgaattgatgttaccct
cataaagcacgtggcctcttatcgagaaagaaattaccgtcgctcgtg
atttgtttgcaaaaagaacaaaactgaaaaaacccagacacgctcgac
ttcctgtcttcctattgattgcagcttccaatttcgtcacacaacaag
gtcctagcgacggctcacaggttttgtaacaagcaatcgaaggttctg
gaatggcgggaaagggtttagtaccacatgctatgatgcccactgtga
tctccagagcaaagttcgttcgatcgtactgttactctctctctttca
aacagaattgtccgaatcgtgtgacaacaacagcctgttctcacacac
tcttttcttctaaccaagggggtggtttagtttagtagaacctcgtga
aacttacatttacatatatataaacttgcataaattggtcaatgcaag
aaatacatatttggtcttttctaattcgtagtttttcaagttcttaga
tgctttctttttctcttttttacagatcatcaaggaagtaattatcta
ctttttacaacaaatataaaacaaggatccgtaatacgactcactata
gggcccgggcgtcgacatggaacagaagttgatttccgaagaagacct
cgagtaagcttggtaccgcggctagctaagatccgctctaaccgaaaa
ggaaggagttagacaacctgaagtctaggtccctatttatttttttat
agttatgttagtattaagaacgttatttatatttcaaatttttctttt
ttttctgtacagacgcgtgtacgcatgtaacattatactgaaaacctt
gcttgagaaggttttgggacgctcgaagatccagctgcattaatgaat
cggccaacgcgcggggagaggcggtttgcgtattgggcgctcttccgc
ttcctcgctcactgactcgctgcgctcggtcgttcggctgcggcgagc
ggtatcagctcactcaaaggcggtaatacggttatccacagaatcagg
ggataacgcaggaaagaacatgtgagcaaaaggccagcaaaaggccag
gaaccgtaaaaaggccgcgttgctggcgtttttccataggctccgccc
ccctgacgagcatcacaaaaatcgacgctcaagtcagaggtggcgaaa
cccgacaggactataaagataccaggcgtttccccctggaagctccct
cgtgcgctctcctgttccgaccctgccgcttaccggatacctgtccgc
ctttctcccttcgggaagcgtggcgctttctcatagctcacgctgtag
gtatctcagttcggtgtaggtcgttcgctccaagctgggctgtgtgca
cgaaccccccgttcagcccgaccgctgcgccttatccggtaactatcg
tcttgagtccaacccggtaagacacgacttatcgccactggcagcagc
cactggtaacaggattagcagagcgaggtatgtaggcggtgctacaga
gttcttgaagtggtggcctaactacggctacactagaaggacagtatt
tggtatctgcgctctgctgaagccagttaccttcggaaaaagagttgg
tagctcttgatccggcaaacaaaccaccgctggtagcggtggtttttt
tgtttgcaagcagcagattacgcgcagaaaaaaaggatctcaagaaga
tcctttgatcttttctacggggtctgacgctcagtggaacgaaaactc
acgttaagggattttggtcatgagattatcaaaaaggatcttcaccta
gatccttttaaattaaaaatgaagttttaaatcaatctaaagtatata
tgagtaaacttggtctgacagttaccaatgcttaatcagtgaggcacc
tatctcagcgatctgtctatttcgttcatccatagttgcctgactccc
cgtcgtgtagataactacgatacgggagggcttaccatctggccccag
tgctgcaatgataccgcgagacccacgctcaccggctccagatttatc
agcaataaaccagccagccggaagggccgagcgcagaagtggtcctgc
aactttatccgcctccatccagtctattaattgttgccgggaagctag
agtaagtagttcgccagttaatagtttgcgcaacgttgttgccattgc
tacaggcatcgtggtgtcacgctcgtcgtttggtatggcttcattcag
ctccggttcccaacgatcaaggcgagttacatgatcccccatgttgtg
caaaaaagcggttagctccttcggtcctccgatcgttgtcagaagtaa
gttggccgcagtgttatcactcatggttatggcagcactgcataattc
tcttactgtcatgccatccgtaagatgcttttctgtgactggtgagta
ctcaaccaagtcattctgagaatagtgtatgcggcgaccgagttgctc
ttgcccggcgtcaatacgggataataccgcgccacatagcagaacttt
aaaagtgctcatcattggaaaacgttcttcggggcgaaaactctcaag
gatcttaccgctgttgagatccagttcgatgtaacccactcgtgcacc
caactgatcttcagcatcttttactttcaccagcgtttctgggtgagc
aaaaacaggaaggcaaaatgccgcaaaaaagggaataagggcgacacg
gaaatgttgaatactcatactcttcctttttcaatattattgaagcat
ttatcagggttattgtctcatgagcggatacatatttgaatgtattta
gaaaaataaacaaataggggttccgcgcacatttccccgaaaagtgcc
acctgaacgaagcatctgtgcttcattttgtagaacaaaaatgcaacg
cgagagcgctaatttttcaaacaaagaatctgagctgcatttttacag
aacagaaatgcaacgcgaaagcgctattttaccaacgaagaatctgtg
cttcatttttgtaaaacaaaaatgcaacgcgagagcgctaatttttca
aacaaagaatctgagctgcatttttacagaacagaaatgcaacgcgag
agcgctattttaccaacaaagaatctatacttcttttttgttctacaa
aaatgcatcccgagagcgctatttttctaacaaagcatcttagattac
tttttttctcctttgtgcgctctataatgcagtctcttgataactttt
tgcactgtaggtccgttaaggttagaagaaggctactttggtgtctat
tttctcttccataaaaaaagcctgactccacttcccgcgtttactgat
tactagcgaagctgcgggtgcattttttcaagataaaggcatccccga
ttatattctataccgatgtggattgcgcatactttgtgaacagaaagt
gatagcgttgatgattcttcattggtcagaaaattatgaacggtttct
tctattttgtctctatatactacgtataggaaatgtttacattttcgt
attgttttcgattcactctatgaatagttcttactacaatttttttgt
ctaaagagtaatactagagataaacataaaaaatgtagaggtcgagtt
tagatgcaagttcaaggagcgaaaggtggatgggtaggttatataggg
atatagcacagagatatatagcaaagagatacttttgagcaatgtttg
tggaagcggtattcgcaatattttagtagctcgttacagtccggtgcg
tttttggttttttgaaagtgcgtcttcagagcgcttttggttttcaaa
agcgctctgaagttcctatactttctagagaataggaacttcggaata
ggaacttcaaagcgtttccgaaaacgagcgcttccgaaaatgcaacgc
gagctgcgcacatacagctcactgttcacgtcgcacctatatctgcgt
gttgcctgtatatatatatacatgagaagaacggcatagtgcgtgttt
atgcttaaatgcgtacttatatgcgtctatttatgtaggatgaaaggt
agtctagtacctcctgtgatattatcccattccatgcggggtatcgta
tgcttccttcagcactaccctttagctgttctatatgctgccactcct
caattggattagtctcatccttcaatgctatcatttcctttgatattg
gatcatactaagaaaccattattatcatgacattaacctataaaaata
ggcgtatcacgaggccctttcgtc (SEQ ID NO: 194). The
capitalized sequence is SEQ ID NO: 195.

TABLE 38
Nucleotide Sequence of OxyS-Q299L in the pSP-G1
plasmid in FY251 strain.

Sequence for OxyS from Streptomyces rimosus
with a Q299L mutation is shown capitalized
within the context of the pSP-G1 backbone
containing pTEF1, the FLAG tag and tADH1
(Addgene 64736) (partial, decapitalized).
tcgcgcgtttcggtgatgacggtgaaaacctctgacacatgcagctc
ccggagacggtcacagcttgtctgtaagcggatgccgggagcagaca
agcccgtcagggcgcgtcagcgggtgttggcgggtgtcggggctggc
ttaactatgcggcatcagagcagattgtactgagagtgcaccatacc
acagcttttcaattcaattcatcattttttttttattcttttttttg
atttcggtttctttgaaatttttttgattcggtaatctccgaacaga
aggaagaacgaaggaaggagcacagacttagattggtatatatacgc
atatgtagtgttgaagaaacatgaaattgcccagtattcttaaccca
actgcacagaacaaaaacctgcaggaaacgaagataaatcatgtcga
aagctacatataaggaacgtgctgctactcatcctagtcctgttgct
gccaagctatttaatatcatgcacgaaaagcaaacaaacttgtgtgc
ttcattggatgttcgtaccaccaaggaattactggagttagttgaag
cattaggtcccaaaatttgtttactaaaaacacatgtggatatcttg
actgatttttccatggagggcacagttaagccgctaaaggcattatc
cgccaagtacaattttttactcttcgaagacagaaaatttgctgaca
ttggtaatacagtcaaattgcagtactctgcgggtgtatacagaata
gcagaatgggcagacattacgaatgcacacggtgtggtgggcccagg
tattgttagcggtttgaagcaggcggcagaagaagtaacaaaggaac
ctagaggccttttgatgttagcagaattgtcatgcaagggctcccta
tctactggagaatatactaagggtactgttgacattgcgaagagcga
caaagattttgttatcggctttattgctcaaagagacatgggtggaa
gagatgaaggttacgattggttgattatgacacccggtgtgggttta
gatgacaagggagacgcattgggtcaacagtatagaaccgtggatga
tgtggtctctacaggatctgacattattattgttggaagaggactat
ttgcaaagggaagggatgctaaggtagagggtgaacgttacagaaaa
gcaggctgggaagcatatttgagaagatgcggccagcaaaactaaaa
aactgtattataagtaaatgcatgtatactaaactcacaaattagag
cttcaatttaattatatcagttattaccctatgcggtgtgaaatacc
gcacagatgcgtaaggagaaaataccgcatcaggaaattgtaaacgt
taatattttgttaaaattcgcgttaaatttttgttaaatcagctcat
tttttaaccaataggccgaaatcggcaaaatcccttataaatcaaaa
gaatagaccgagatagggttgagtgttgttccagtttggaacaagag
tccactattaaagaacgtggactccaacgtcaaagggcgaaaaaccg
tctatcagggcgatggcccactacgtgaaccatcaccctaatcaagt
tttttggggtcgaggtgccgtaaagcactaaatcggaaccctaaagg
gagcccccgatttagagcttgacggggaaagccggcgaacgtggcga
gaaaggaagggaagaaagcgaaaggagcgggcgctagggcgctggca
agtgtagcggtcacgctgcgcgtaaccaccacacccgccgcgcttaa
tgcgccgctacagggcgcgtccattcgccattcaggctgcgcaactg
ttgggaagggcgatcggtgcgggcctcttcgctattacgccagctga
attggagcgacctcatgctatacctgagaaagcaacctgacctacag
gaaagagttactcaagaataagaattttcgttttaaaacctaagagt
cactttaaaatttgtatacacttattttttttataacttatttaata
ataaaaatcataaatcataagaaattcgcttatttagaagtgtcaac
aacgtatctaccaacgatttgacccttttccatcttttcgtaaattt
ctggcaaggtagacaagccgacaaccttgattggagacttgaccaaa
cctctggcgaagaattgttaattaagagctcagatcttatcgtcgtc
atccttgtaatccatcgatacCATTGCAGGTGGGCCGAACCAAGTAC
GCAGCGCGTCTGTCAATCCATCAGTACCAGTTTCTGGGGCAGCTGTC
CAACAAATGTAACCATCAGGCCTTACAAGTAAAGCAGTTAAGCCATC
TACTGCAGAACCAGGGTCCGCCACACCTTCAGCTGCAACTAAATCAA
CCCTATCTTTCCAAGCAGCGGCTTCATCTGCTAGTGGTTGATCACAA
CCTAACGTTAATAATACGCCACGTGCAGCATGGAAATAAGTGGACAA
CTGTCTTGTTCCATCTTCGGTGACAAGAGGTAAGTCGGGTACTCTTC
TTCCCAGCAATGGATGTTCCCCAGCCATTGGGTATCTAACGTCTAAT
GCGGAGATTAGCCCCGCTAAATATCTATTAGTCTCAGGAACGTGCAA
CAATTCAATCATTAATTCCCTTAAACCCTCGTAGCGAGGATCTGGAT
CAATTAGAACAGCTTGAGCCCTTGTATTTCTCAAAACACCGGCGGCT
ATCGGCCTTCTCTCAGCTTCATACGTATCCAATAACTCTGGTGGAGC
GGTCCCGGCAATGGTAGCACCAAGCTTCCAACCCAAGTTAACGGCAT
CTTGAAATCCTAAGTTCAGTCCCAAGCCACCAGCGGGCAAATGAATA
TGGCATGCATCACCAGCCAAAAGAACTCTCCCATCTCTATACCTTTC
TGCTTGTCTACTTGTATCGGTCAAACGAGATAACCATCTTACATCAT
GCATTCCAAAGTCAGATCCAGCAACTTCTGTTAACGCGGCTCTGACA
TCCTCCTCCGTTACCGGCGCCCTCCTGTCAGCATACGGTCTATCGAA
AAAGGCGACTGTTGCTCTGTAGACGTCTGGTTCTAGCGGAAATGCTG
CGAACCATGCACGAAGGTCATGTCTCATGACACCATAAGGACGCATA
GGACCCATACCTTCTCCGTGAGGAAGCTCTTCTCTGAATCTTGCGTC
TGCGATGACAGCAAACATATGTGGGTCTTGACCTGGAAAATCTATGC
CCAATAGTTTCCTCACCGTACTGCCTCCACCGTCGCAGCCCACCAAA
TAAGCACATTCTACTTCGTATGGGCCTTCAGGGCCTGTCACGCTCAC
AGTAACACCATCAGCATCTTGTTCCAAGGCGGTCACGGCATGACCAC
GACGCAGCTCCGCCCCTAGCTCCAAAGCTCTACCTGTTAGCAGTTCT
TCAGTTCGTACTTGTGGAACAAACAATGCATATGGGTGTCTAGTATC
AAATGATGAGAAATCCAGGGGGACGCCTAATGAGGCAAAATTACCAC
CTCTTAACTTTGGTGCTTCAGCCTGGAATCCTTCACCGAGGCCTCTC
ATATCTAATAGTTCAACAGTGCGAGCGTGGACTCCTAGAGCTTTCGA
GAAGTCAACAGGCTCGGCTAATCTTTCTAAAACCAAAGTTCTGGCAC
CCGCCAGCCGAAGTTCACATGCTAACATCAAACCGGTGGGTCCTGCA
CCAGCTATAACAACATCGTACCTCATgcggccgcttgtaattaaaac
ttagattagattgctatgctttctttctaatgagcaagaagtaaaaa
aagttgtaatagaacaagaaaaatgaaactgaaacttgagaaattga
agaccgtttattaacttaaatatcaatgggaggtcatcgaaagagaa
aaaaatcaaaaaaaaaaattttcaagaaaaagaaacgtgataaaaat
ttttattgcctttttcgacgaagaaaaagaaacgaggcggtctcttt
tttcttttccaaacctttagtacgggtaattaacgacaccctagagg
aagaaagaggggaaatttagtatgctgtgcttgggtgttttgaagtg
gtacggcgatgcgcggagtccgagaaaatctggaagagtaaaaaagg
agtagaaacattttgaagctatggtgtgtgcggccggcctggaagta
ccttcaaagaatggggtcttatcttgttttgcaagtaccactgagca
ggataataatagaaatgataatatactatagtagagataacgtcgat
gacttcccatactgtaattgcttttagttgtgtatttttagtgtgca
agtttctgtaaatcgattaatttttttttctttcctctttttattaa
ccttaatttttattttagattcctgacttcaactcaagacgcacaga
tattataacatctgcataataggcatttgcaagaattactcgtgagt
aaggaaagagtgaggaactatcgcatacctgcatttaaagatgccga
tttgggcgcgaatcctttattttggcttcaccctcatactattatca
gggccagaaaaaggaagtgtttccctccttcttgaattgatgttacc
ctcataaagcacgtggcctcttatcgagaaagaaattaccgtcgctc
gtgatttgtttgcaaaaagaacaaaactgaaaaaacccagacacgct
cgacttcctgtcttcctattgattgcagcttccaatttcgtcacaca
acaaggtcctagcgacggctcacaggttttgtaacaagcaatcgaag
gttctggaatggcgggaaagggtttagtaccacatgctatgatgccc
actgtgatctccagagcaaagttcgttcgatcgtactgttactctct
ctctttcaaacagaattgtccgaatcgtgtgacaacaacagcctgtt
ctcacacactcttttcttctaaccaagggggtggtttagtttagtag
aacctcgtgaaacttacatttacatatatataaacttgcataaattg
gtcaatgcaagaaatacatatttggtcttttctaattcgtagttttt
caagttcttagatgctttctttttctcttttttacagatcatcaagg
aagtaattatctactttttacaacaaatataaaacaaggatccgtaa
tacgactcactatagggcccgggcgtcgacatggaacagaagttgat
ttccgaagaagacctcgagtaagcttggtaccgcggctagctaagat
ccgctctaaccgaaaaggaaggagttagacaacctgaagtctaggtc
cctatttatttttttatagttatgttagtattaagaacgttatttat
atttcaaatttttcttttttttctgtacagacgcgtgtacgcatgta
acattatactgaaaaccttgcttgagaaggttttgggacgctcgaag
atccagctgcattaatgaatcggccaacgcgcggggagaggcggttt
gcgtattgggcgctcttccgcttcctcgctcactgactcgctgcgct
cggtcgttcggctgcggcgagcggtatcagctcactcaaaggcggta
atacggttatccacagaatcaggggataacgcaggaaagaacatgtg
agcaaaaggccagcaaaaggccaggaaccgtaaaaaggccgcgttgc
tggcgtttttccataggctccgcccccctgacgagcatcacaaaaat
cgacgctcaagtcagaggtggcgaaacccgacaggactataaagata
ccaggcgtttccccctggaagctccctcgtgcgctctcctgttccga
ccctgccgcttaccggatacctgtccgcctttctcccttcgggaagc
gtggcgctttctcatagctcacgctgtaggtatctcagttcggtgta
ggtcgttcgctccaagctgggctgtgtgcacgaaccccccgttcagc
ccgaccgctgcgccttatccggtaactatcgtcttgagtccaacccg
gtaagacacgacttatcgccactggcagcagccactggtaacaggat
tagcagagcgaggtatgtaggcggtgctacagagttcttgaagtggt
ggcctaactacggctacactagaaggacagtatttggtatctgcgct
ctgctgaagccagttaccttcggaaaaagagttggtagctcttgatc
cggcaaacaaaccaccgctggtagcggtggtttttttgtttgcaagc
agcagattacgcgcagaaaaaaaggatctcaagaagatcctttgatc
ttttctacggggtctgacgctcagtggaacgaaaactcacgttaagg
gattttggtcatgagattatcaaaaaggatcttcacctagatccttt
taaattaaaaatgaagttttaaatcaatctaaagtatatatgagtaa
acttggtctgacagttaccaatgcttaatcagtgaggcacctatctc
agcgatctgtctatttcgttcatccatagttgcctgactccccgtcg
tgtagataactacgatacgggagggcttaccatctggccccagtgct
gcaatgataccgcgagacccacgctcaccggctccagatttatcagc
aataaaccagccagccggaagggccgagcgcagaagtggtcctgcaa
ctttatccgcctccatccagtctattaattgttgccgggaagctaga
gtaagtagttcgccagttaatagtttgcgcaacgttgttgccattgc
tacaggcatcgtggtgtcacgctcgtcgtttggtatggcttcattca
gctccggttcccaacgatcaaggcgagttacatgatcccccatgttg
tgcaaaaaagcggttagctccttcggtcctccgatcgttgtcagaag
taagttggccgcagtgttatcactcatggttatggcagcactgcata
attctcttactgtcatgccatccgtaagatgcttttctgtgactggt
gagtactcaaccaagtcattctgagaatagtgtatgcggcgaccgag
ttgctcttgcccggcgtcaatacgggataataccgcgccacatagca
gaactttaaaagtgctcatcattggaaaacgttcttcggggcgaaaa
ctctcaaggatcttaccgctgttgagatccagttcgatgtaacccac
tcgtgcacccaactgatcttcagcatcttttactttcaccagcgttt
ctgggtgagcaaaaacaggaaggcaaaatgccgcaaaaaagggaata
agggcgacacggaaatgttgaatactcatactcttcctttttcaata
ttattgaagcatttatcagggttattgtctcatgagcggatacatat
ttgaatgtatttagaaaaataaacaaataggggttccgcgcacattt
ccccgaaaagtgccacctgaacgaagcatctgtgcttcattttgtag
aacaaaaatgcaacgcgagagcgctaatttttcaaacaaagaatctg
agctgcatttttacagaacagaaatgcaacgcgaaagcgctatttta
ccaacgaagaatctgtgcttcatttttgtaaaacaaaaatgcaacgc
gagagcgctaatttttcaaacaaagaatctgagctgcatttttacag
aacagaaatgcaacgcgagagcgctattttaccaacaaagaatctat
acttcttttttgttctacaaaaatgcatcccgagagcgctatttttc
taacaaagcatcttagattactttttttctcctttgtgcgctctata
atgcagtctcttgataactttttgcactgtaggtccgttaaggttag
aagaaggctactttggtgtctattttctcttccataaaaaaagcctg
actccacttcccgcgtttactgattactagcgaagctgcgggtgcat
tttttcaagataaaggcatccccgattatattctataccgatgtgga
ttgcgcatactttgtgaacagaaagtgatagcgttgatgattcttca
ttggtcagaaaattatgaacggtttcttctattttgtctctatatac
tacgtataggaaatgtttacattttcgtattgttttcgattcactct
atgaatagttcttactacaatttttttgtctaaagagtaatactaga
gataaacataaaaaatgtagaggtcgagtttagatgcaagttcaagg
agcgaaaggtggatgggtaggttatatagggatatagcacagagata
tatagcaaagagatacttttgagcaatgtttgtggaagcggtattcg
caatattttagtagctcgttacagtccggtgcgtttttggttttttg
aaagtgcgtcttcagagcgcttttggttttcaaaagcgctctgaagt
tcctatactttctagagaataggaacttcggaataggaacttcaaag
cgtttccgaaaacgagcgcttccgaaaatgcaacgcgagctgcgcac
atacagctcactgttcacgtcgcacctatatctgcgtgttgcctgta
tatatatatacatgagaagaacggcatagtgcgtgtttatgcttaaa
tgcgtacttatatgcgtctatttatgtaggatgaaaggtagtctagt
acctcctgtgatattatcccattccatgcggggtatcgtatgcttcc
ttcagcactaccctttagctgttctatatgctgccactcctcaattg
gattagtctcatccttcaatgctatcatttcctttgatattggatca
tactaagaaaccattattatcatgacattaacctataaaaataggcg
tatcacgaggccctttcgtc (SEQ ID NO: 196). The
capitalized sequence is SEQ ID NO: 197.

TABLE 39
Nucleotide Sequence of wild-type OxyS.

ATGAGGTACGATGTTGTTATAGCTGGTGCAGGACCCACCGGTTTGATGT
TAGCATGTGAACTTCGGCTGGCGGGTGCCAGAACTTTGGTTTTAGAAAG
ATTAGCCGAGCCTGTTGACTTCTCGAAAGCTCTAGGAGTCCACGCTCGC
ACTGTTGAACTATTAGATATGAGAGGCCTCGGTGAAGGATTCCAGGCTG
AAGCACCAAAGTTAAGAGGTGGTAATTTTGCCTCATTAGGCGTCCCCCT
GGATTTCTCATCATTTGATACTAGACACCCATATGCATTGTTTGTTCCA
CAAGTACGAACTGAAGAACTGCTAACAGGTAGAGCTTTGGAGCTAGGGG
CGGAGCTGCGTCGTGGTCATGCCGTGACCGCCTTGGAACAAGATGCTGA
TGGTGTTACTGTGAGCGTGACAGGCCCTGAAGGCCCATACGAAGTAGAA
TGTGCTTATTTGGTGGGCTGCGACGGTGGAGGCAGTACGGTGAGGAAAC
TATTGGGCATAGATTTTCCAGGTCAAGACCCACATATGTTTGCTGTCAT
CGCAGACGCAAGATTCAGAGAAGAGCTTCCTCACGGAGAAGGTATGGGT
CCTATGCGTCCTTATGGTGTCATGAGACATGACCTTCGTGCATGGTTCG
CAGCATTTCCGCTAGAACCAGACGTCTACAGAGCAACAGTCGCCTTTTT
CGATAGACCGTATGCTGACAGGAGGGCGCCGGTAACGGAGGAGGATGTC
AGAGCCGCGTTAACAGAAGTTGCTGGATCTGACTTTGGAATGCATGATG
TAAGATGGTTATCTCGTTTGACCGATACAAGTAGACAAGCAGAAAGGTA
TAGAGATGGGAGAGTTCTTTTGGCTGGTGATGCATGCCATATTCATTTG
CCCGCTGGTGGCCAGGGACTGAACTTAGGATTTCAAGATGCCGTTAACT
TGGGTTGGAAGCTTGGTGCTACCATTGCCGGGACCGCTCCACCAGAGTT
ATTGGATACGTATGAAGCTGAGAGAAGGCCGATAGCCGCCGGTGTTTTG
AGAAATACAAGGGCTCAAGCTGTTCTAATTGATCCAGATCCTCGCTACG
AGGGTTTAAGGGAATTAATGATTGAATTGTTGCACGTTCCTGAGACTAA
TAGATATTTAGCGGGGCTAATCTCCGCATTAGACGTTAGATACCCAATG
GCTGGGGAACATCCATTGCTGGGAAGAAGAGTACCCGACTTACCTCTTG
TCACCGAAGATGGAACAAGACAGTTGTCCACTTATTTCCATGCTGCACG
TGGCGTATTATTAACGTTAGGTTGTGATCAACCACTAGCAGATGAAGCC
GCTGCTTGGAAAGATAGGGTTGATTTAGTTGCAGCTGAAGGTGTGGCGG
ACCCTGGTTCTGCAGTAGATGGCTTAACTGCTTTACTTGTAAGGCCTGA
TGGTTACATTTGTTGGACAGCTGCCCCAGAAACTGGTACTGATGGATTG
ACAGACGCGCTGCGTACTTGGTTCGGCCCACCTGCAATGgtatcgatgg
attacaaggatgacgacgataagatctga (SEQ ID NO: 210).
The capitalized sequence is SEQ ID NO: 211.

TABLE 40
Amino acid Sequences of wild-type
OxyS and OxyS mutants.

| | |
|---|---|
| Wildtype | MRYDVVIAGAGPTGLMLACELRLAGARTLVLERLAEPVD |
| OxyS | FSKALGVHARTVELLDMRGLGEGFQAEAPKLRGGNFASL |
| | GVPLDFSSFDTRHPYALFVPQVRTEELLTGRALELGAEL |
| | RRGHAVTALEQDADGVTVSVTGPEGPYEVECAYLVGCDG |
| | GGSTVRKLLGIDFPGQDPHMFAVIADARFREELPHGEGM |
| | GPMRPYGVMRHDLRAWFAAFPLEPDVYRATVAFFDRPYA |
| | DRRAPVTEEDVRAALTEVAGSDFGMHDVRWLSRLTDTSR |
| | QAERYRDGRVLLAGDACHIHLPAGGQGLNLGFQDAVNLG |
| | WKLGATIAGTAPPELLDTYEAERRPIAAGVLRNTRAQAV |
| | LIDPDPRYEGLRELMIELLHVPETNRYLAGLISALDVRY |
| | PMAGEHPLLGRRVPDLPLVTEDGTRQLSTYFHAARGVLL |
| | TLGCDQPLADEAAAWKDRVDLVAAEGVADPGSAVDGLTA |
| | LLVRPDGYICWTAAPETGTDGLTDALRTWFGPPAM |
| | (SEQ ID NO: 212) |
| OxyS L44F | MRYDVVIAGAGPTGLMLACELRLAGARTLVLERLAEPVD |
| | FSKAFGVHARTVELLDMRGLGEGFQAEAPKLRGGNFASL |
| | GVPLDFSSFDTRHPYALFVPQVRTEELLTGRALELGAEL |
| | RRGHAVTALEQDADGVTVSVTGPEGPYEVECAYLVGCDG |
| | GGSTVRKLLGIDFPGQDPHMFAVIADARFREELPHGEGM |
| | GPMRPYGVMRHDLRAWFAAFPLEPDVYRATVAFFDRPYA |
| | DRRAPVTEEDVRAALTEVAGSDFGMHDVRWLSRLTDTSR |
| | QAERYRDGRVLLAGDACHIHLPAGGQGLNLGFQDAVNLG |
| | WKLGATIAGTAPPELLDTYEAERRPIAAGVLRNTRAQAV |
| | LIDPDPRYEGLRELMIELLHVPETNRYLAGLISALDVRY |
| | PMAGEHPLLGRRVPDLPLVTEDGTRQLSTYFHAARGVLL |
| | TLGCDQPLADEAAAWKDRVDLVAAEGVADPGSAVDGLTA |
| | LLVRPDGYICWTAAPETGTDGLTDALRTWFGPPAMVSMD |
| | YKDDDDKI (SEQ ID NO: 213) |
| OxyS | MRYDVVIAGAGPTGLMLACELRLAGARTLVLERLAEPVD |
| G45A | FSKALAVHARTVELLDMRGLGEGFQAEAPKLRGGNFASL |
| | GVPLDFSSFDTRHPYALFVPQVRTEELLTGRALELGAEL |
| | RRGHAVTALEQDADGVTVSVTGPEGPYEVECAYLVGCDG |
| | GGSTVRKLLGIDFPGQDPHMFAVIADARFREELPHGEGM |
| | GPMRPYGVMRHDLRAWFAAFPLEPDVYRATVAFFDRPYA |
| | DRRAPVTEEDVRAALTEVAGSDFGMHDVRWLSRLTDTSR |
| | QAERYRDGRVLLAGDACHIHLPAGGQGLNLGFQDAVNLG |
| | WKLGATIAGTAPPELLDTYEAERRPIAAGVLRNTRAQAV |
| | LIDPDPRYEGLRELMIELLHVPETNRYLAGLISALDVRY |
| | PMAGEHPLLGRRVPDLPLVTEDGTRQLSTYFHAARGVLL |
| | TLGCDQPLADEAAAWKDRVDLVAAEGVADPGSAVDGLTA |
| | LLVRPDGYICWTAAPETGTDGLTDALRTWFGPPAMVSMD |
| | YKDDDDKI (SEQ ID NO: 214) |
| OxyS | MRYDVVIAGAGPTGLMLACELRLAGARTLVLERLAEPVD |
| Q299L | FSKALGVHARTVELLDMRGLGEGFQAEAPKLRGGNFASL |
| | GVPLDFSSFDTRHPYALFVPQVRTEELLTGRALELGAEL |
| | RRGHAVTALEQDADGVTVSVTGPEGPYEVECAYLVGCDG |
| | GGSTVRKLLGIDFPGQDPHMFAVIADARFREELPHGEGM |
| | GPMRPYGVMRHDLRAWFAAFPLEPDVYRATVAFFDRPYA |
| | DRRAPVTEEDVRAALTEVAGSDFGMHDVRWLSRLTDTSR |
| | QAERYRDGRVLLAGDACHIHLPAGGLGLNLGFQDAVNLG |
| | WKLGATIAGTAPPELLDTYEAERRPIAAGVLRNTRAQAV |
| | LIDPDPRYEGLRELMIELLHVPETNRYLAGLISALDVRY |
| | PMAGEHPLLGRRVPDLPLVTEDGTRQLSTYFHAARGVLL |
| | TLGCDQPLADEAAAWKDRVDLVAAEGVADPGSAVDGLTA |
| | LLVRPDGYICWTAAPETGTDGLTDALRTWFGPPAMVSMD |
| | YKDDDDKI (SEQ ID NO: 215) |

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The contents of all figures and all references, patents and published patent applications and Accession numbers cited throughout this application are expressly incorporated herein by reference.

1. A fungal cell genetically engineered to produce a therapeutic molecule in situ, wherein the therapeutic molecule is secreted from the fungal cell.

2. The genetically-engineered fungal cell of claim 1, wherein:
(a) the therapeutic molecule is secreted from the fungal cell by a secretory pathway of the fungal cell;
(b) the fungal cell expresses a heterologous efflux pump;
(c) the genetically-engineered fungal cell secretes multiple therapeutic molecules;
(d) the therapeutic molecule is selected from the group consisting of a peptide, a small molecule and a combination thereof;
(e) the genetically-engineered fungal cell heterologously expresses a protein involved in the biosynthesis pathway of the therapeutic molecule; and/or
(f) the fungal cell is Saccharomyces cerevisiae or Saccharomyces boulardii.

3.-5. (canceled)

6. The genetically-engineered fungal cell of claim 2, wherein the therapeutic molecule is a small molecule and/or the small molecule has anti-inflammatory and/or antibiotic properties.

7.-8. (canceled)

9. The genetically-engineered fungal cell of claim 6, wherein the small molecule is TAN-1612 or a derivative thereof.

10.-11. (canceled)

12. The genetically-engineered fungal cell of claim 10, wherein the protein involved in the biosynthesis pathway of the therapeutic molecule is an enzyme selected from the group consisting of a transferase, a synthase, a lactamase, a monooxygenase, a reductase, a hydroxylase, an oxidoreductase, a glycotransferase, a fusion protein thereof and a combination thereof.

13. The genetically-engineered fungal cell of claim 12, wherein the enzyme is selected from the group consisting of AdaA, AdaB, AdaC, AdaD, NpgA and a combination thereof.

14. The genetically-engineered fungal cell of claim 13, further comprising an enzyme for modifying TAN-1612 to synthesize a TAN-1612 analogue.

15. The genetically-engineered fungal cell of claim 14, wherein the enzyme for modifying TAN-1612 is (i) selected from the group consisting of a monooxygenase, a reductase, a hydroxylase, an oxidoreductase, a glycotransferase, a fusion protein thereof and a combination thereof and/or (ii) selected from the group consisting of PgaE, DacO1, DacO4, PgaE, SsfO1, CtcN, CtcM, FNO, OxyS, a fusion protein thereof and a combination thereof.

16.-17. (canceled)

18. The genetically-engineered fungal cell of claim 15, wherein OxyS is a OxyS mutant comprising one or more mutations at amino acids K42, A43, L44, G45, L95, F96, M176, W211, F212, T225, A227, F228, V240, P295, A296, G297, G298, G299, N302, I353, D354, R358, V372, P375 or a combination thereof.

19. The genetically-engineered fungal cell of claim 13, further comprising an enzyme for modifying TAN-1612 to synthesize tetracycline or an analogue thereof.

20.-21. (canceled)

22. The genetically-engineered fungal cell of claim 5, wherein the therapeutic molecule is a peptide and/or the peptide has anti-fungal and/or antibiotic properties.

23. (canceled)

24. The genetically-engineered fungal cell of claim 22, wherein the peptide is a toxin peptide.

25. (canceled)

26. The genetically-engineered fungal cell of claim 24, wherein the toxin peptide is a K1, K2 or K28 toxin peptide derived from Saccharomyces cerevisiae.

27.-28. (canceled)

29. A method for treating a subject in need thereof comprising administering to the subject a fungal cell genetically engineered to generate and secrete a therapeutic molecule in situ for treating the subject.

30. The method of claim 29, wherein:
(a) the therapeutic molecule is secreted from the genetically-engineered fungal cell by a secretory pathway of the genetically-engineered fungal cell;
(b) the genetically-engineered fungal cell expresses a heterologous efflux pump;
(c) the genetically-engineered fungal cell is a live genetically-engineered fungal cell;
(d) the genetically-engineered fungal cell secretes multiple therapeutic molecules;
(e) the therapeutic molecule is selected from the group consisting of a peptide, a small molecule and a combination thereof; and/or
(f) the genetically-engineered fungal cell heterologously expresses a protein involved in the biosynthesis pathway of the therapeutic molecule.

31.-34. (canceled)

35. The method of claim 30, wherein the therapeutic molecule is a small molecule and/or the small molecule has anti-inflammatory and/or antibiotic properties.

36. (canceled)

37. The method of claim 35, wherein the small molecule is used to treat an infection selected from the group consisting of intraabdominal infections, respiratory infections, bacterial infections, urinary tract infections, urethral infections, cervical infections and rectal infections.

38. The method of claim 35, wherein the small molecule is TAN-1612 or a derivative thereof.

39.-40. (canceled)

41. The method of claim 30, wherein the protein involved in the biosynthesis pathway of the therapeutic molecule is an enzyme selected from the group consisting of a transferase, a synthase, a lactamase, a monooxygenase, a reductase, a hydroxylase, an oxidoreductase, a glycotransferase, a fusion protein thereof and a combination thereof.

42. The method of claim 41, wherein the enzyme is selected from the group consisting of AdaA, AdaB, AdaC, AdaD, NpgA and a combination thereof.

43. The method of claim 41, further comprising an enzyme for modifying TAN-1612 to synthesize a TAN-1612 analogue.

44. The method of claim 43, wherein the enzyme for modifying TAN-1612 is (i) selected from the group consisting of a monooxygenase, a reductase, a hydroxylase, an oxidoreductase, a glycotransferase, a fusion protein thereof and a combination thereof and/or (ii) selected from the group consisting of consisting of PgaE, DacO1, DacO4, PgaE, SsfO1, CtcN, CtcM, FNO, OxyS, a fusion protein thereof and a combination thereof.

45.-46. (canceled)

47. The method of claim 44, wherein OxyS is a OxyS mutant comprising one or more mutations at amino acids K42, A43, L44, G45, L95, F96, M176, W211, F212, T225, A227, F228, V240, P295, A296, G297, G298, G299, N302, I353, D354, R358, V372, P375 or a combination thereof.

48. The method of claim 42, further comprising an enzyme for modifying TAN-1612 to synthesize tetracycline or an analogue.

49.-51. (canceled)

52. The method of claim 30, wherein the peptide is a fungal toxin peptide.

53. The method of claim 52, wherein the fungal toxin peptide is a K1, K2 or K28 toxin peptide derived from Saccharomyces cerevisiae.

54.-55. (canceled)

56. The method of claim 29, wherein the genetically-engineered fungal cell is one or more of the following:
(a) formulated for parenteral administration, intraocular administration, intraaural administration, intranasal administration, oral administration, rectal administration, vaginal administration or topical administration;
(b) not administered to the digestive system;
(c) administered to the subject to treat an infection; and/or
(d) Saccharomyces cerevisiae or Saccharomyces boulardii.

57.-58. (canceled)

59. A pharmaceutical composition comprising one or more genetically-engineered fungal cells of claim 1 and a pharmaceutically acceptable carrier.

60. The pharmaceutical composition of claim 59, wherein the pharmaceutical composition formulated for parenteral administration, intraocular administration, intraaural administration, intranasal administration, oral administration, rectal administration, vaginal administration or topical administration.

61. An OxyS protein comprising one or more mutations of an amino acid selected from the group consisting of K42, A43, L44, G45, L95, F96, M176, W211, F212, T225, A227, F228, V240, P295, A296, G297, G298, G299, N302, I353, D354, R358, V372, P375 and a combination thereof.
